(19) United States
(12) Patent Application Publication
(10) Pub. No.: US 2013/0207092 A1
(43) Pub. Date: Aug. 15, 2013

(54) COMPOUND FOR ORGANIC PHOTOELECTRIC DEVICE AND ORGANIC PHOTOELECTRIC DEVICE INCLUDING THE SAME

(71) Applicants: Dal-Ho HUH, Uiwang-si (KR); Sung-Hyun JUNG, Uiwang-si (KR); Dong-Wan RYU, Uiwang-si (KR); Kyoung-Mi LEE, Uiwang-si (KR); Mi-Young CHAE, Uiwang-si (KR)

(72) Inventors: Dal-Ho HUH, Uiwang-si (KR); Sung-Hyun JUNG, Uiwang-si (KR); Dong-Wan RYU, Uiwang-si (KR); Kyoung-Mi LEE, Uiwang-si (KR); Mi-Young CHAE, Uiwang-si (KR)

(21) Appl. No.: 13/804,478

(22) Filed: Mar. 14, 2013

Related U.S. Application Data
(63) Continuation of application No. PCT/KR2011/001798, filed on Mar. 15, 2011.

(30) Foreign Application Priority Data
Sep. 20, 2010 (KR) 10-2010-0092535

(51) Int. Cl. H01L 51/00

(52) U.S. Cl. CPC H01L 51/0073; H01L 51/0072; H01L 51/0054; H01L 51/0058
USPC 257/40; 548/440

(57) ABSTRACT

A compound for an organic photoelectric device is represented by the following Chemical Formula 1:

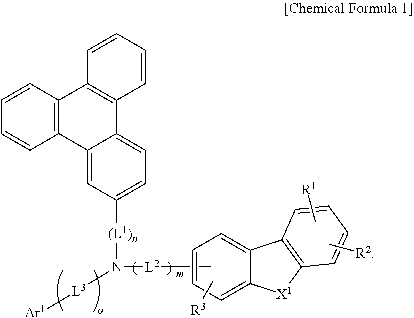

### CROSS-REFERENCE TO RELATED APPLICATIONS

This application is a continuation of pending International Application No. PCT/KR2011/001798, entitled “Compound for Organic Photoelectric Device and Organic Photoelectric Device Including the Same,” which was filed on Mar. 15, 2011, the entire contents of which are hereby incorporated by reference.

This application claims priority under 35 U.S.C. §119 to Korean Patent Application No. 10-2010-0092535, filed on Sep. 20, 2010, in the Korean Intellectual Property Office, and entitled: “Compound for Organic Photoelectric Device and Organic Photoelectric Device Including the Same,” which is incorporated by reference herein in its entirety.

### BACKGROUND

1. Field

Embodiments relate to a compound for an organic photoelectric device and an organic photoelectric device including the same.

2. Description of the Related Art

An organic photoelectric device is, in a broad sense, a device for transforming photo-energy to electrical energy or conversely, a device for transforming electrical energy to photo-energy. An organic photoelectric device may be classified as follows in accordance with its driving principles. A first organic photoelectric device is an electronic device driven as follows: excitons are generated in an organic material layer by photons from an external light source; the excitons are separated into electrons and holes; and the electrons and holes are transferred to different electrodes as a current source (voltage source). A second organic photoelectric device is an electronic device driven as follows: a voltage or a current is applied to at least two electrodes to inject holes and/or electrons into an organic material semiconductor positioned at an interface of the electrodes, and the device is driven by the injected electrons and holes. Particularly, an organic light emitting diode (OLED) has recently drawn attention due to an increasing demand for a flat panel display. In general, organic light emission refers to conversion of electrical energy into photo-energy.

### SUMMARY

Embodiments are directed to a compound for an organic photoelectric device, the compound being represented by the following Chemical Formula 1:

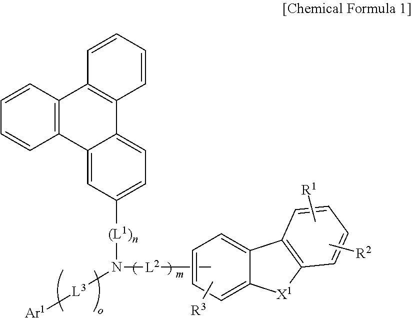

In Chemical Formula 1,

L¹ to L³ may each independently be selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,

n, m, and o may each independently be integers ranging from 1 to 4,

X¹ may be selected from the group of NR′, O, S, and P, an R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group,

Ar¹ may be a substituted or unsubstituted C6 to C30 aryl group or a substituted or unsubstituted C2 to C30 heteroaryl group, and

R¹ to R³ may each independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

X¹ may be NR′, and R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

Ar¹ may be selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group.

Embodiments are also directed to a compound for an organic photoelectric device, the compound being represented by the following Chemical Formula 2:

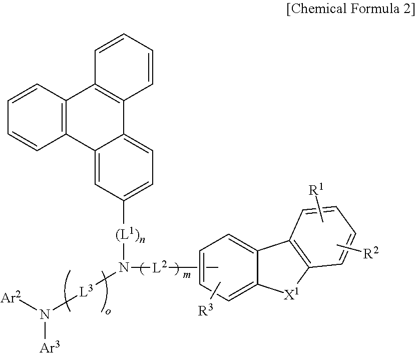

In Chemical Formula 2,

L¹ to L³ may each independently be selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,

n, m, and o may each independently be integers ranging from 1 to 4,

X¹ may be selected from the group of NR′, O, S, and P, and R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group,

Ar² and Ar³ may each independently be selected from the group of a substituted or unsubstituted C1 to C6 alkyl group, a substituted or unsubstituted C6 to C30 aryl group, and a substituted or unsubstituted C2 to C30 heteroaryl group, and

R¹ to R³ may each independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

X¹ may be NR′, and R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group

Ar² and Ar³ may each independently be selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group.

Embodiments are also directed to a compound for an organic photoelectric device, the compound being represented by the following Chemical Formula 3:

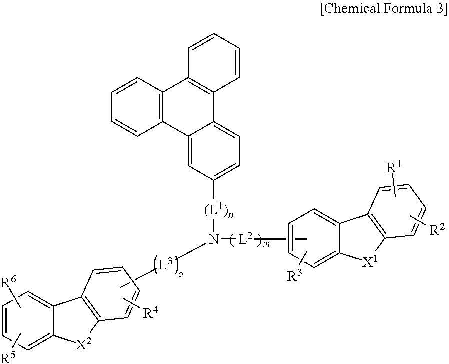

In Chemical Formula 3,

L¹ to L³ may each independently be selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,

n, m, and o may each independently be integers ranging from 1 to 4,

X¹ and X² may each independently be selected from the group of NR′, O, S, and P, wherein R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group, and

R¹ to R⁶ may each independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

X¹ and X² may be NR′, and R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

Embodiments are also directed to a compound for an organic photoelectric device, the compound being represented by one of the following Chemical Formulae 4 to 39, ad1, ad2, k1, or k2:

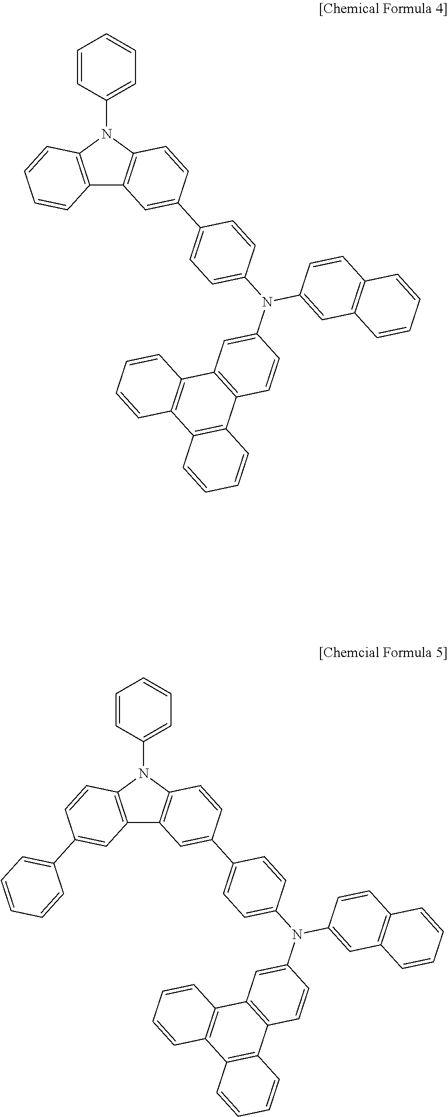 [SMILES: c1ccc(-n2c3ccccc3c3cc(-c4ccc(N(c5ccc6ccccc6c5)c5ccc6c7ccccc7c7ccccc7c6c5)cc4)ccc32)cc1 ; c1ccc(-c2ccc3c(c2)c2cc(-c4ccc(N(c5ccc6ccccc6c5)c5ccc6c7ccccc7c7ccccc7c6c5)cc4)ccc2n3-c2ccccc2)cc1]

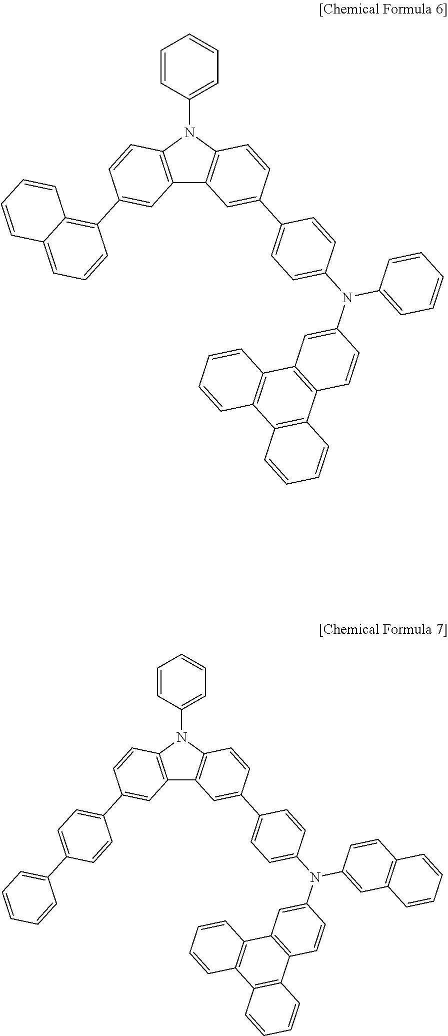 [SMILES: c1ccc(N(c2ccc(-c3ccc4c(c3)c3cc(-c5cccc6ccccc56)ccc3n4-c3ccccc3)cc2)c2ccc3c4ccccc4c4ccccc4c3c2)cc1 ; c1ccc(-c2ccc(-c3ccc4c(c3)c3cc(-c5ccc(N(c6ccc7ccccc7c6)c6ccc7c8ccccc8c8ccccc8c7c6)cc5)ccc3n4-c3ccccc3)cc2)cc1]

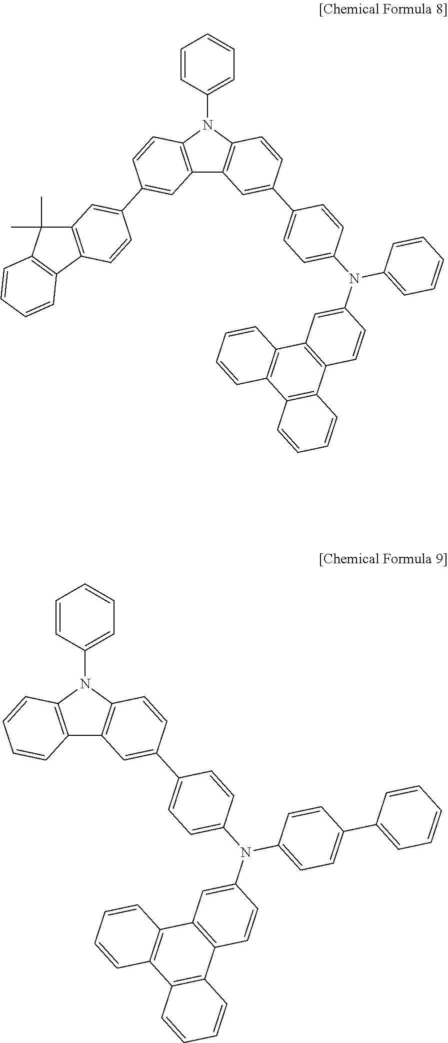 [SMILES: CC1(C)c2ccccc2-c2ccc(-c3ccc4c(c3)c3cc(-c5ccc(N(c6ccccc6)c6ccc7c8ccccc8c8ccccc8c7c6)cc5)ccc3n4-c3ccccc3)cc21 ; c1ccc(-c2ccc(N(c3ccc(-c4ccc5c(c4)c4ccccc4n5-c4ccccc4)cc3)c3ccc4c5ccccc5c5ccccc5c4c3)cc2)cc1]

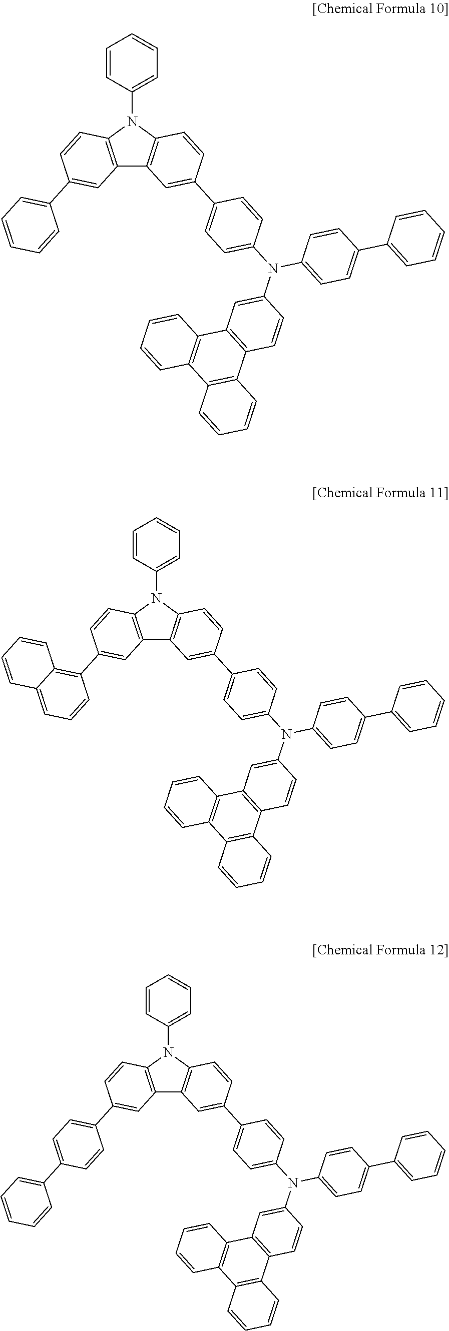 [SMILES: c1ccc(-c2ccc(N(c3ccc(-c4ccc5c(c4)c4cc(-c6ccccc6)ccc4n5-c4ccccc4)cc3)c3ccc4c5ccccc5c5ccccc5c4c3)cc2)cc1 ; c1ccc(-c2ccc(N(c3ccc(-c4ccc5c(c4)c4cc(-c6cccc7ccccc67)ccc4n5-c4ccccc4)cc3)c3ccc4c5ccccc5c5ccccc5c4c3)cc2)cc1 ; c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc2)cc1]

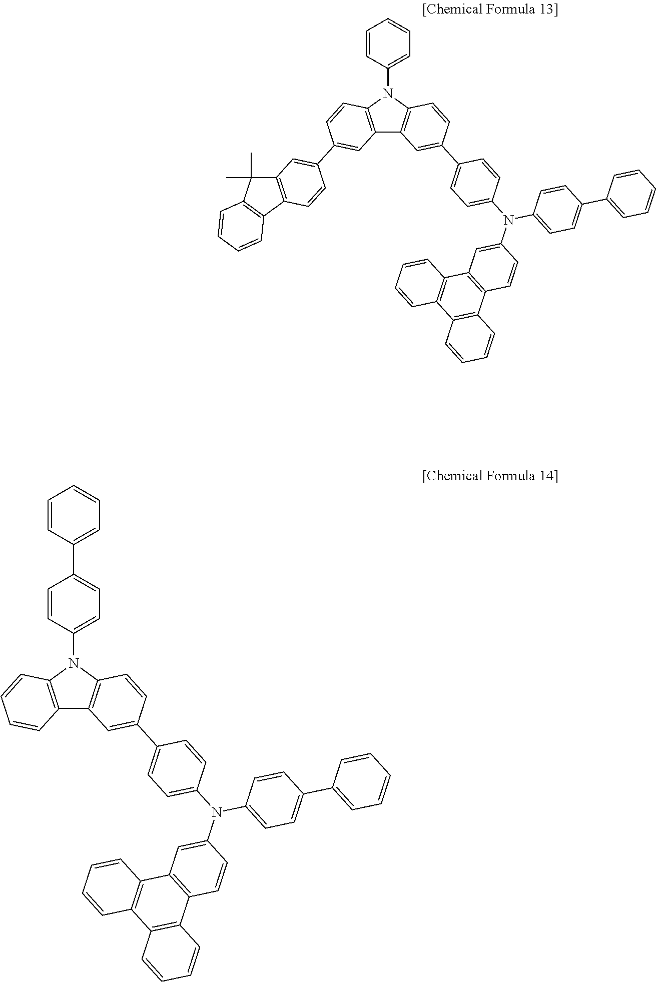 [SMILES: CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc21 ; c1ccc(-c2ccc(N(c3ccc(-c4ccc5c(c4)c4ccccc4n5-c4ccc(-c5ccccc5)cc4)cc3)c3ccc4c5ccccc5c5ccccc5c4c3)cc2)cc1]

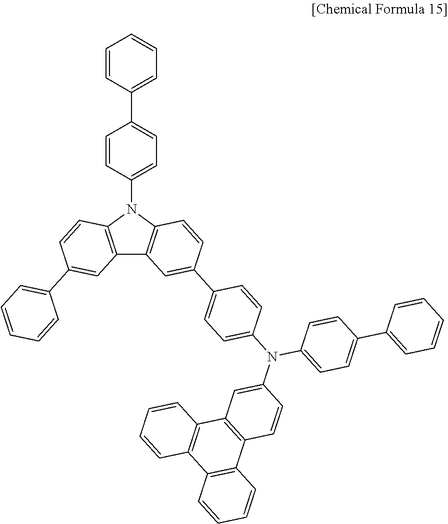 [SMILES: c1ccc(-c2ccc(N(c3ccc(-c4ccc5c(c4)c4cc(-c6ccccc6)ccc4n5-c4ccc(-c5ccccc5)cc4)cc3)c3ccc4c5ccccc5c5ccccc5c4c3)cc2)cc1]

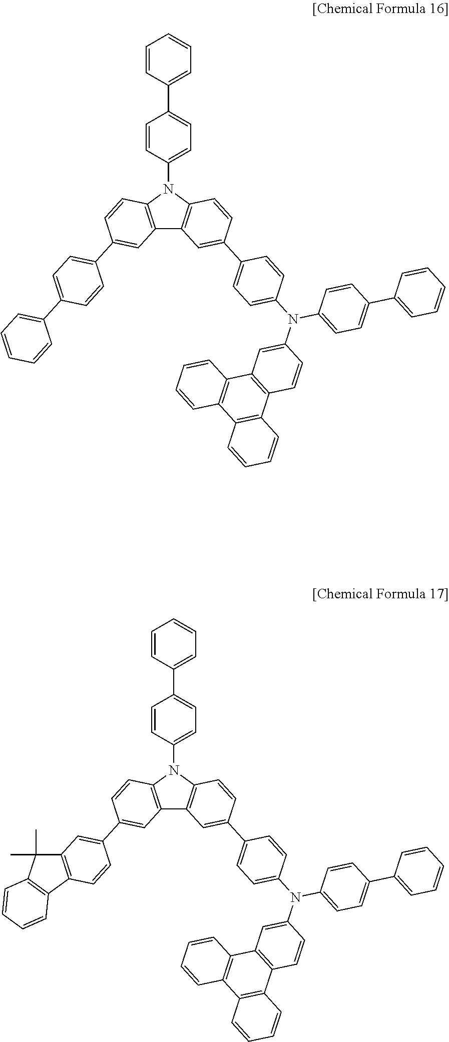 [SMILES: c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc21]

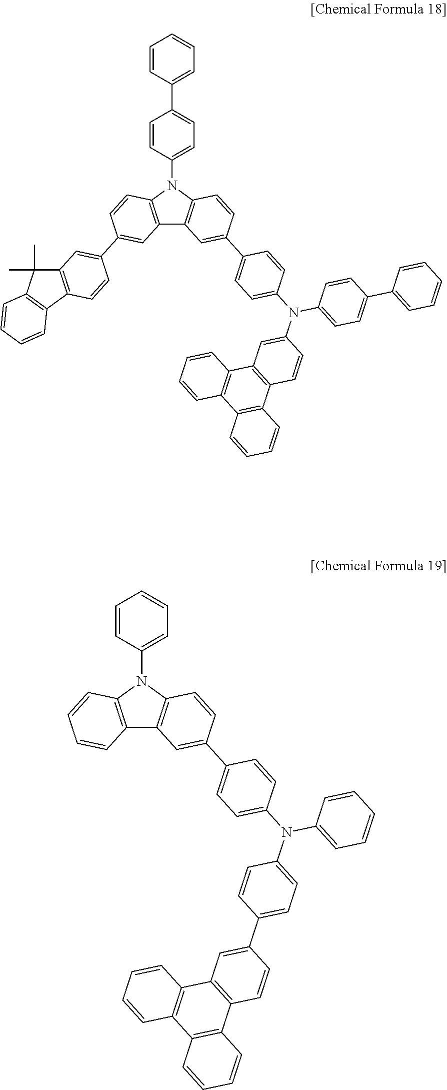 [SMILES: CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc21 ; c1ccc(N(c2ccc(-c3ccc4c5ccccc5c5ccccc5c4c3)cc2)c2ccc(-c3ccc4c(c3)c3ccccc3n4-c3ccccc3)cc2)cc1]

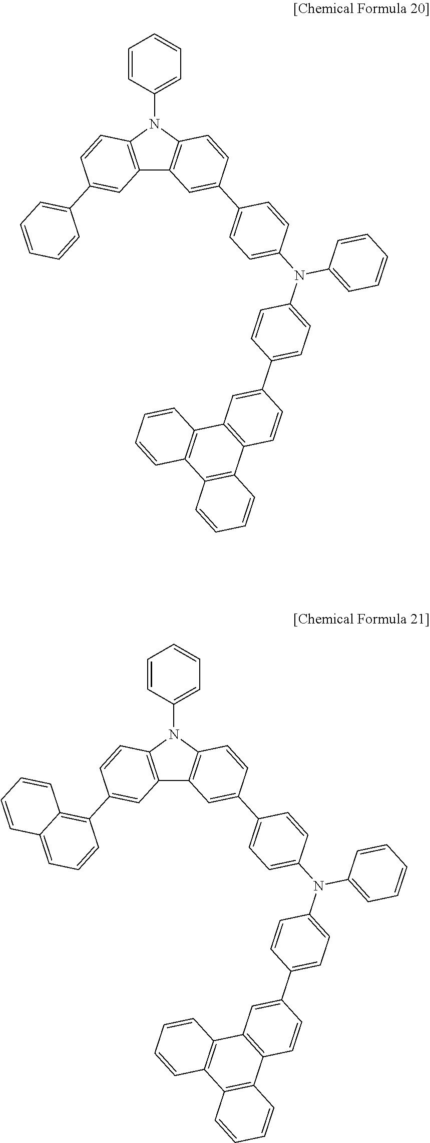 [SMILES: c1ccc(-c2ccc3c(c2)c2cc(-c4ccc(N(c5ccccc5)c5ccc(-c6ccc7c8ccccc8c8ccccc8c7c6)cc5)cc4)ccc2n3-c2ccccc2)cc1 ; c1ccc(N(c2ccc(-c3ccc4c5ccccc5c5ccccc5c4c3)cc2)c2ccc(-c3ccc4c(c3)c3cc(-c5cccc6ccccc56)ccc3n4-c3ccccc3)cc2)cc1]

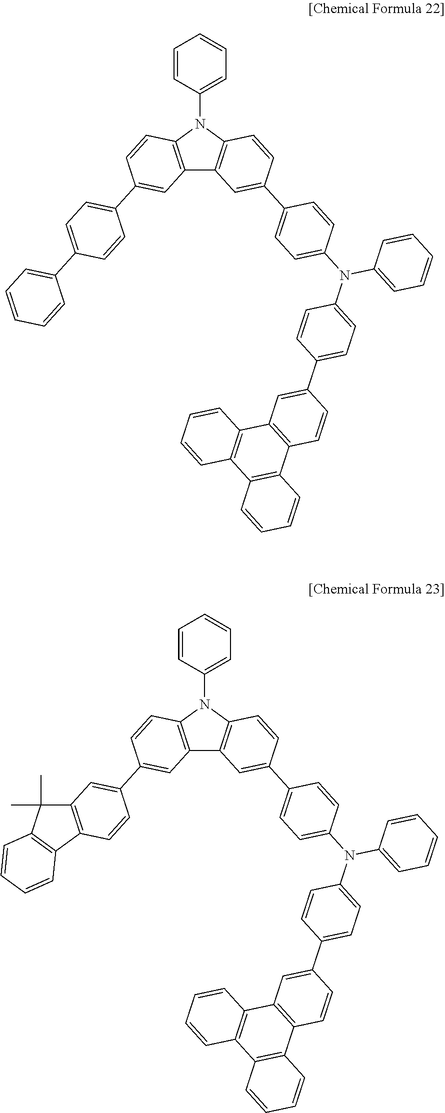 [SMILES: c1ccc(-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc2)cc1 ; CC1(C)c2ccccc2-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc21]

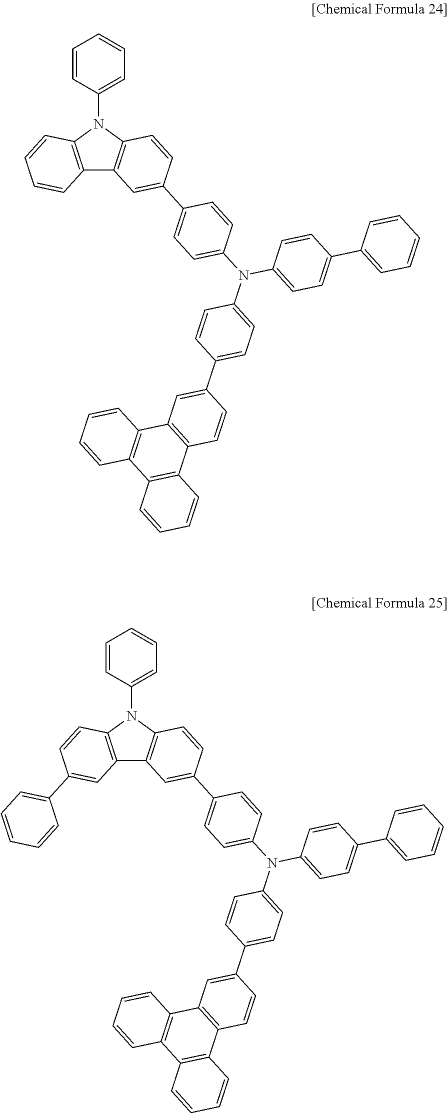 [SMILES: c1ccc(-c2ccc(N(c3ccc(-c4ccc5c6ccccc6c6ccccc6c5c4)cc3)c3ccc(-c4ccc5c(c4)c4ccccc4n5-c4ccccc4)cc3)cc2)cc1 ; c1ccc(-c2ccc(N(c3ccc(-c4ccc5c6ccccc6c6ccccc6c5c4)cc3)c3ccc(-c4ccc5c(c4)c4cc(-c6ccccc6)ccc4n5-c4ccccc4)cc3)cc2)cc1]

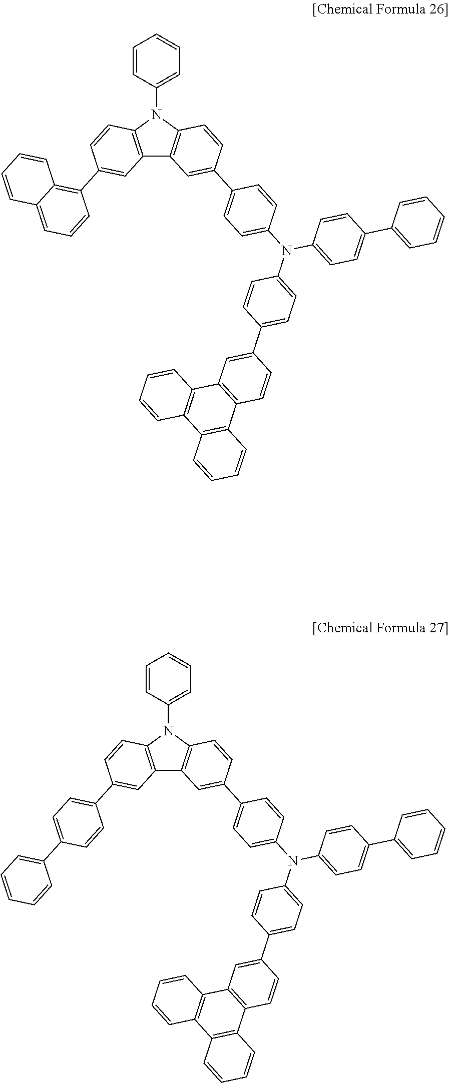 [SMILES: c1ccc(-c2ccc(N(c3ccc(-c4ccc5c6ccccc6c6ccccc6c5c4)cc3)c3ccc(-c4ccc5c(c4)c4cc(-c6cccc7ccccc67)ccc4n5-c4ccccc4)cc3)cc2)cc1 ; c1ccc(-c2ccc(-c3ccc4c(c3)c3cc(-c5ccc(N(c6ccc(-c7ccccc7)cc6)c6ccc(-c7ccc8c9ccccc9c9ccccc9c8c7)cc6)cc5)ccc3n4-c3ccccc3)cc2)cc1]

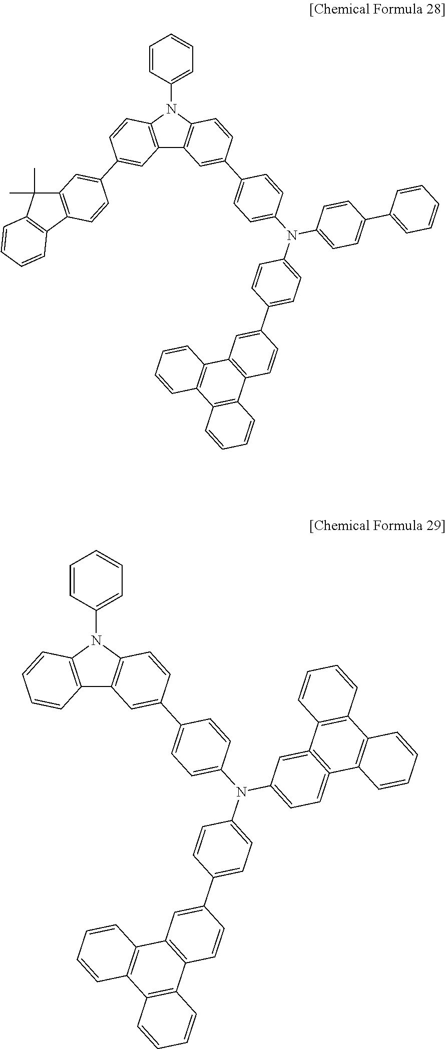

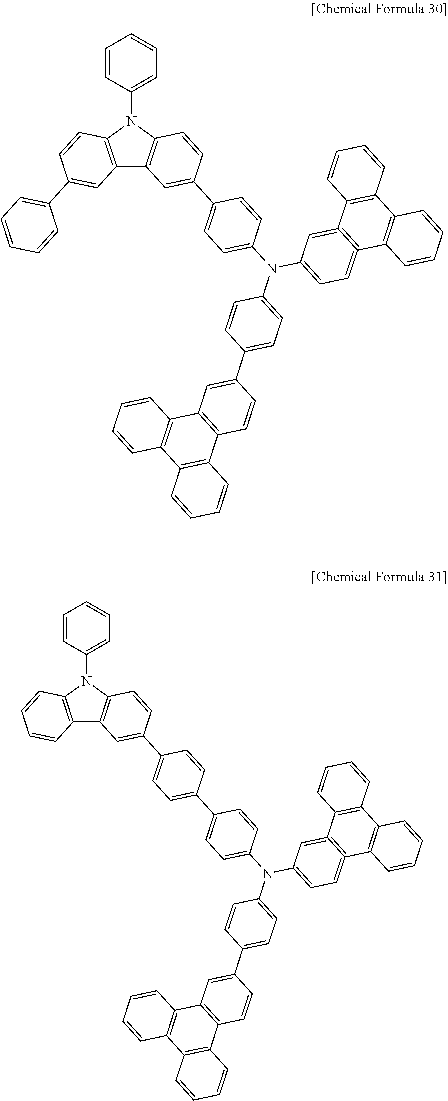

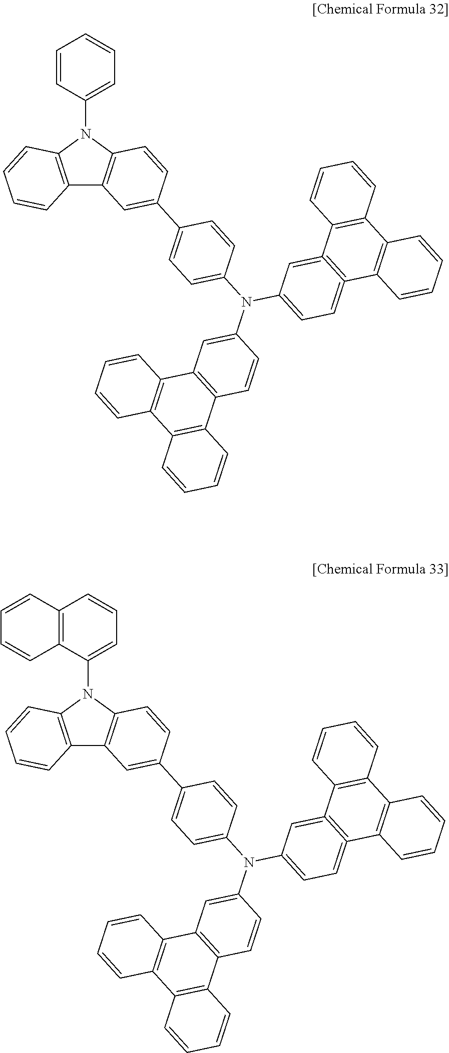

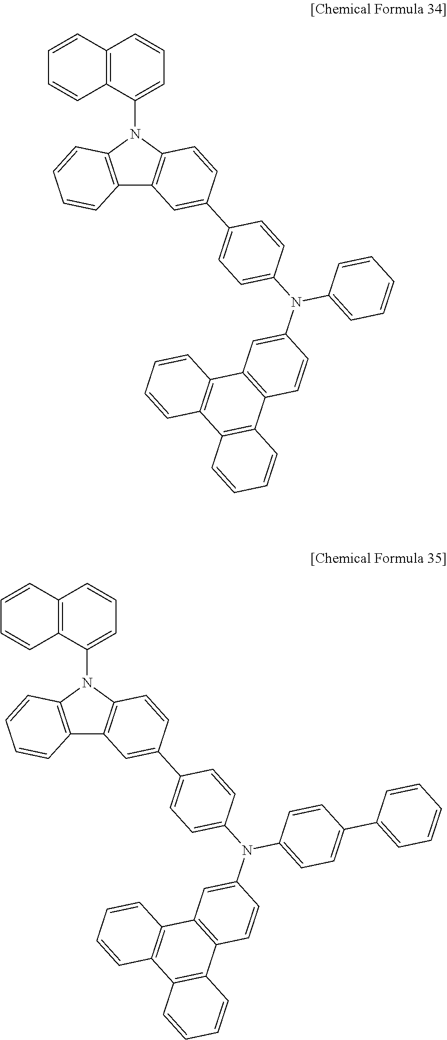 [SMILES: c1ccc(N(c2ccc(-c3ccc4c(c3)c3ccccc3n4-c3cccc4ccccc34)cc2)c2ccc3c4ccccc4c4ccccc4c3c2)cc1 ; c1ccc(-c2ccc(N(c3ccc(-c4ccc5c(c4)c4ccccc4n5-c4cccc5ccccc45)cc3)c3ccc4c5ccccc5c5ccccc5c4c3)cc2)cc1]

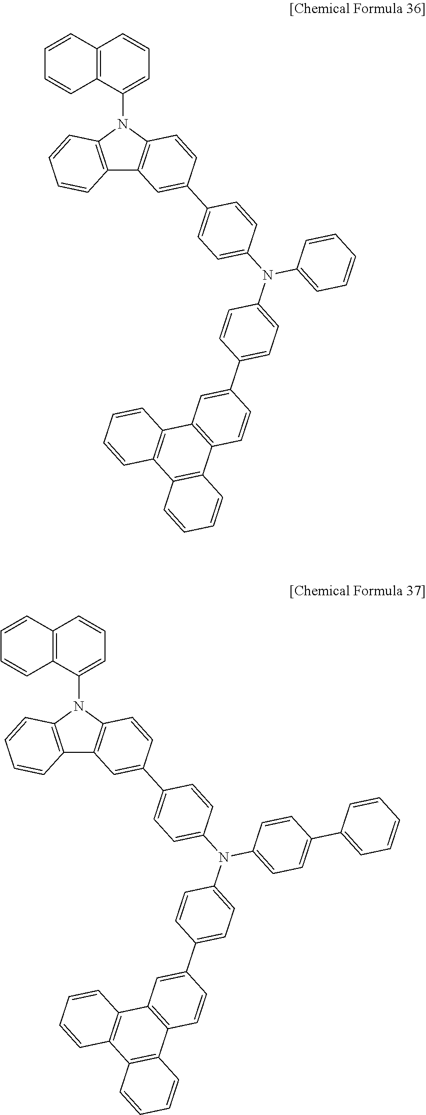 [SMILES: c1ccc(N(c2ccc(-c3ccc4c5ccccc5c5ccccc5c4c3)cc2)c2ccc(-c3ccc4c(c3)c3ccccc3n4-c3cccc4ccccc34)cc2)cc1 ; c1ccc(-c2ccc(N(c3ccc(-c4ccc5c6ccccc6c6ccccc6c5c4)cc3)c3ccc(-c4ccc5c(c4)c4ccccc4n5-c4cccc5ccccc45)cc3)cc2)cc1]

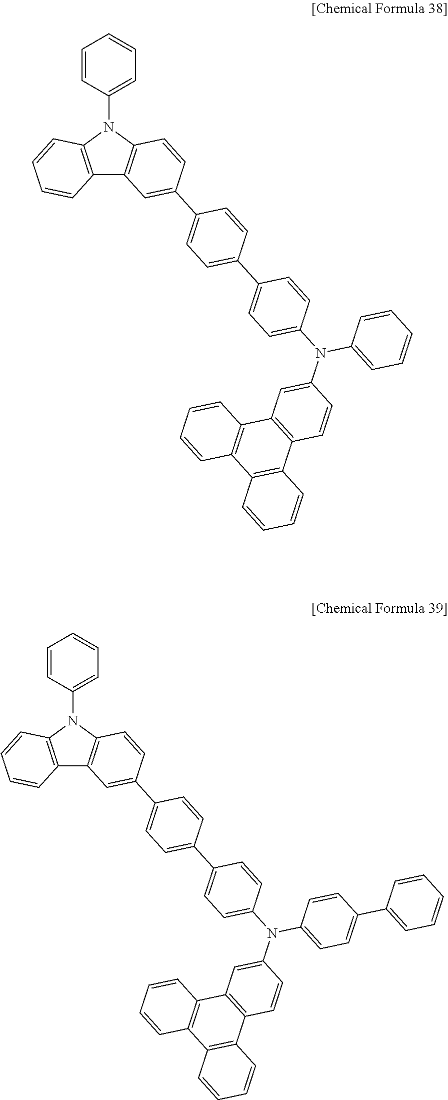 [SMILES: c1ccc(N(c2ccc(-c3ccc(-c4ccc5c(c4)c4ccccc4n5-c4ccccc4)cc3)cc2)c2ccc3c4ccccc4c4ccccc4c3c2)cc1 ; c1ccc(-c2ccc(N(c3ccc(-c4ccc(-c5ccc6c(c5)c5ccccc5n6-c5ccccc5)cc4)cc3)c3ccc4c5ccccc5c5ccccc5c4c3)cc2)cc1]

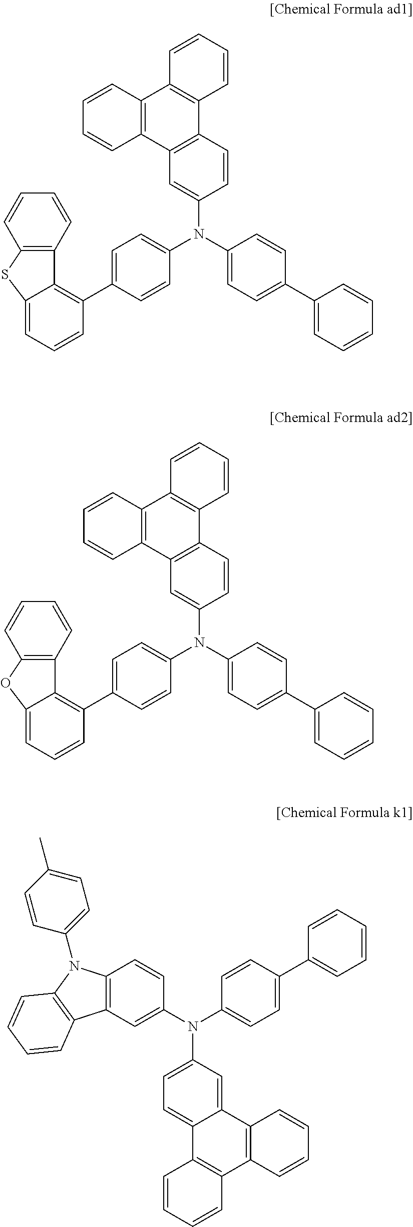 [SMILES: c1ccc(-c2ccc(N(c3ccc(-c4cccc5sc6ccccc6c45)cc3)c3ccc4c5ccccc5c5ccccc5c4c3)cc2)cc1 ; c1ccc(-c2ccc(N(c3ccc(-c4cccc5oc6ccccc6c45)cc3)c3ccc4c5ccccc5c5ccccc5c4c3)cc2)cc1 ; Cc1ccc(-n2c3ccccc3c3cc(N(c4ccc(-c5ccccc5)cc4)c4ccc5c6ccccc6c6ccccc6c5c4)ccc32)cc1]

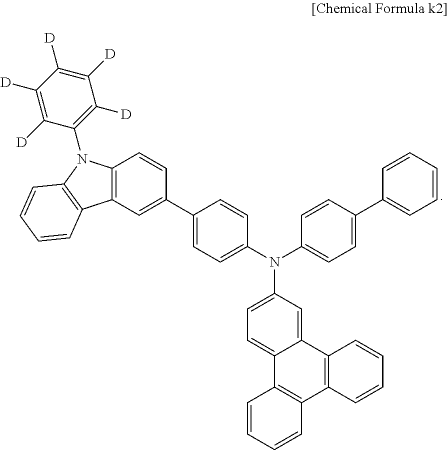 [SMILES: [2H]c1c([2H])c([2H])c(-n2c3ccccc3c3cc(-c4ccc(N(c5ccc(-c6ccccc6)cc5)c5ccc6c7ccccc7c7ccccc7c6c5)cc4)ccc32)c([2H])c1[2H]]

Embodiments are also directed to a compound for an organic photoelectric device, the compound being represented by one of the following Chemical Formulae 40 to 106, ad3, ad4, or ad5:

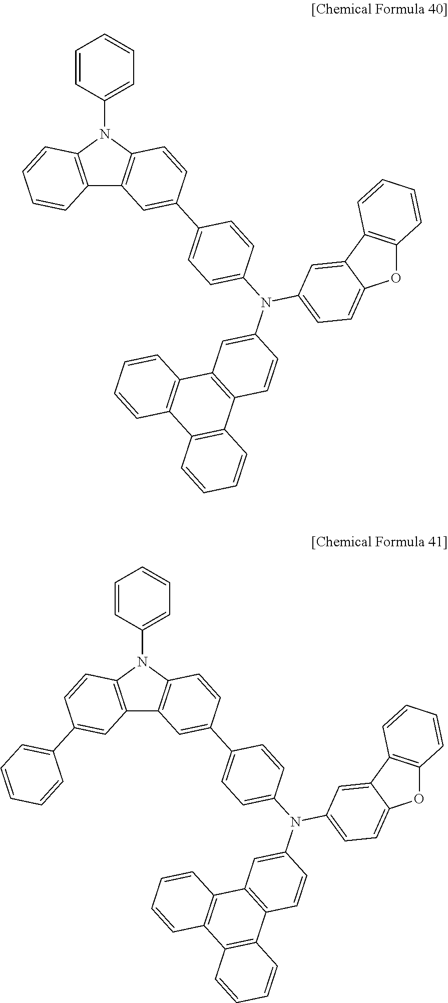 [SMILES: c1ccc(-n2c3ccccc3c3cc(-c4ccc(N(c5ccc6oc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6oc7ccccc7c6c5)c5ccc6c7ccccc7c7ccccc7c6c5)cc4)ccc2n3-c2ccccc2)cc1]

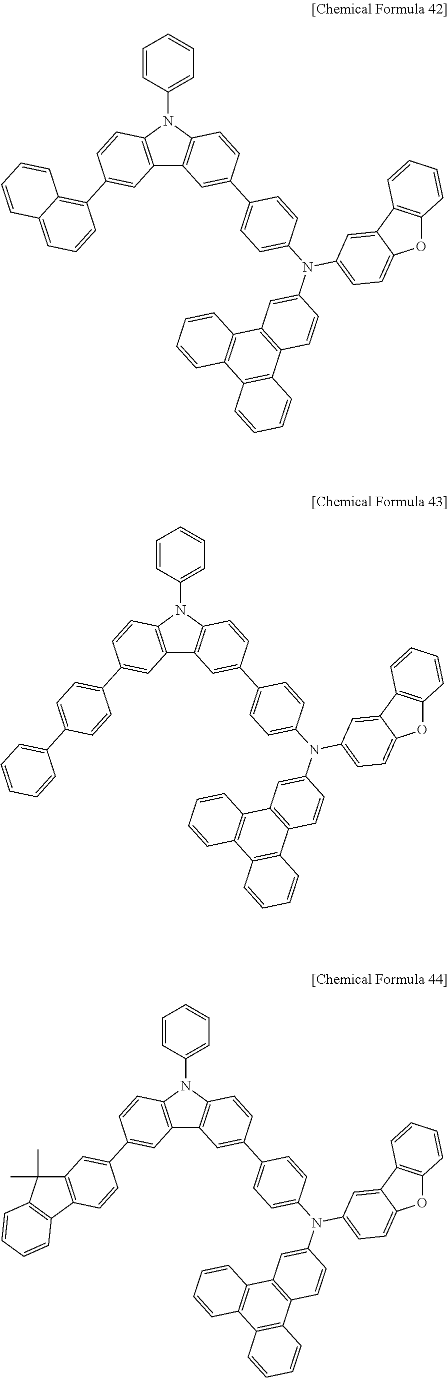 [SMILES: c1ccc(-n2c3ccc(-c4ccc(N(c5ccc6oc7ccccc7c6c5)c5ccc6c7ccccc7c7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc21]

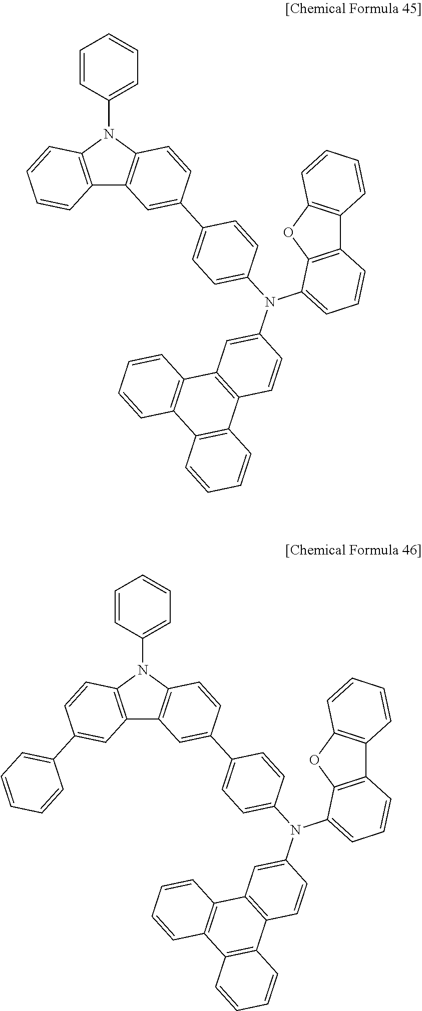 [SMILES: c1ccc(-n2c3ccccc3c3cc(-c4ccc(N(c5ccc6c7ccccc7c7ccccc7c6c5)c5cccc6c5oc5ccccc56)cc4)ccc32)cc1 ; c1ccc(-c2ccc3c(c2)c2cc(-c4ccc(N(c5ccc6c7ccccc7c7ccccc7c6c5)c5cccc6c5oc5ccccc56)cc4)ccc2n3-c2ccccc2)cc1]

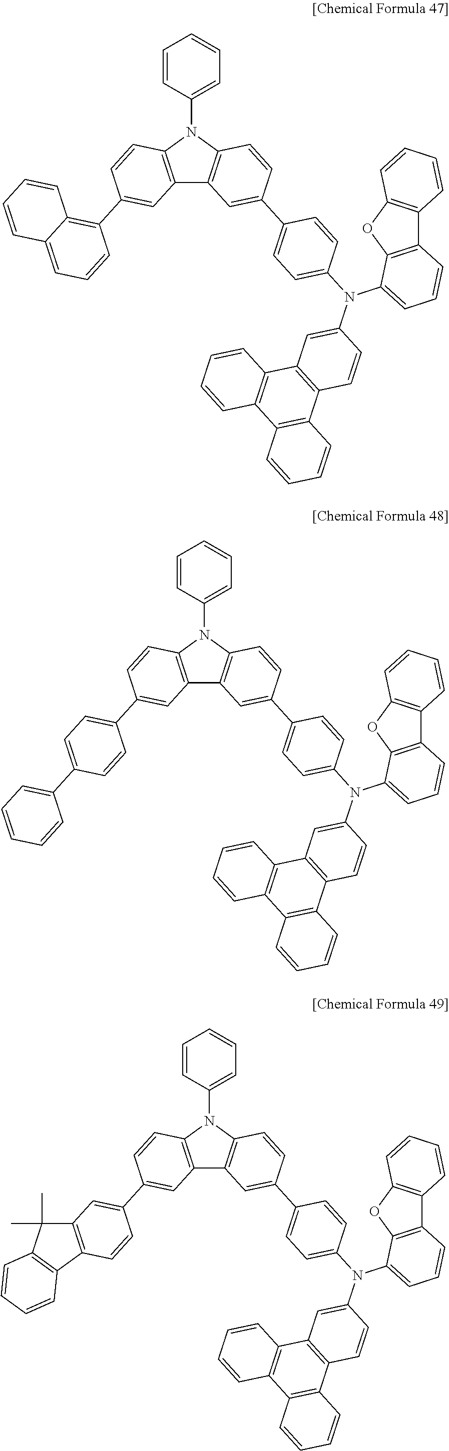 [SMILES: c1ccc(-n2c3ccc(-c4ccc(N(c5ccc6c7ccccc7c7ccccc7c6c5)c5cccc6c5oc5ccccc56)cc4)cc3c3cc(-c4cccc5ccccc45)ccc32)cc1 ; c1ccc(-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccccc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccccc3)cc21]

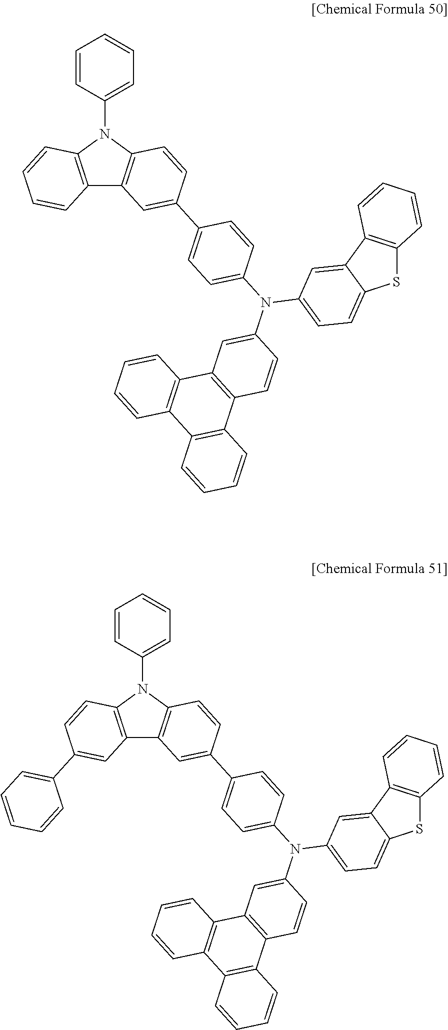 [SMILES: c1ccc(-n2c3ccccc3c3cc(-c4ccc(N(c5ccc6sc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6sc7ccccc7c6c5)c5ccc6c7ccccc7c7ccccc7c6c5)cc4)ccc2n3-c2ccccc2)cc1]

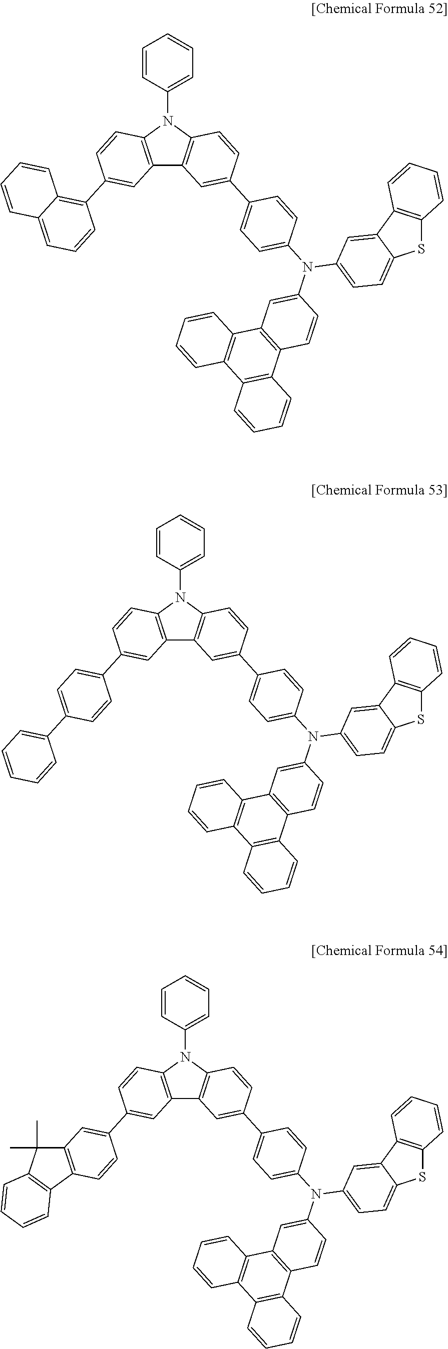 [SMILES: c1ccc(-n2c3ccc(-c4ccc(N(c5ccc6sc7ccccc7c6c5)c5ccc6c7ccccc7c7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc21]

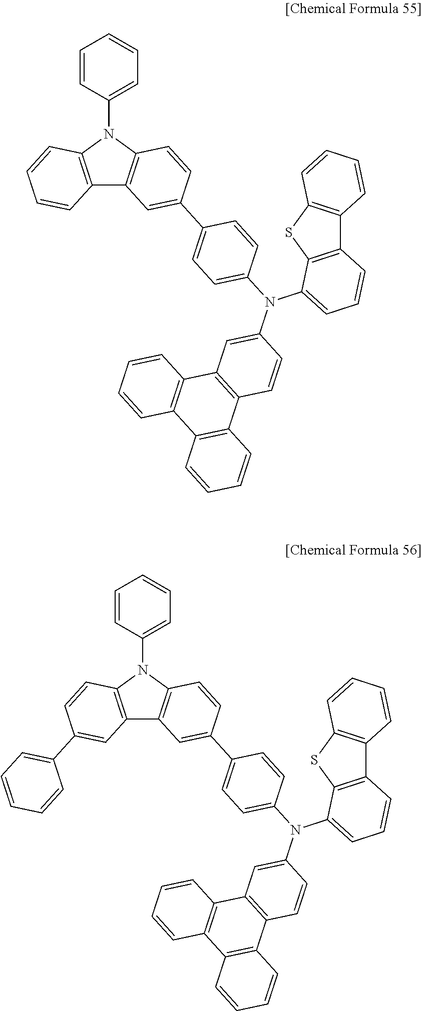 [SMILES: c1ccc(-n2c3ccccc3c3cc(-c4ccc(N(c5ccc6c7ccccc7c7ccccc7c6c5)c5cccc6c5sc5ccccc56)cc4)ccc32)cc1 ; c1ccc(-c2ccc3c(c2)c2cc(-c4ccc(N(c5ccc6c7ccccc7c7ccccc7c6c5)c5cccc6c5sc5ccccc56)cc4)ccc2n3-c2ccccc2)cc1]

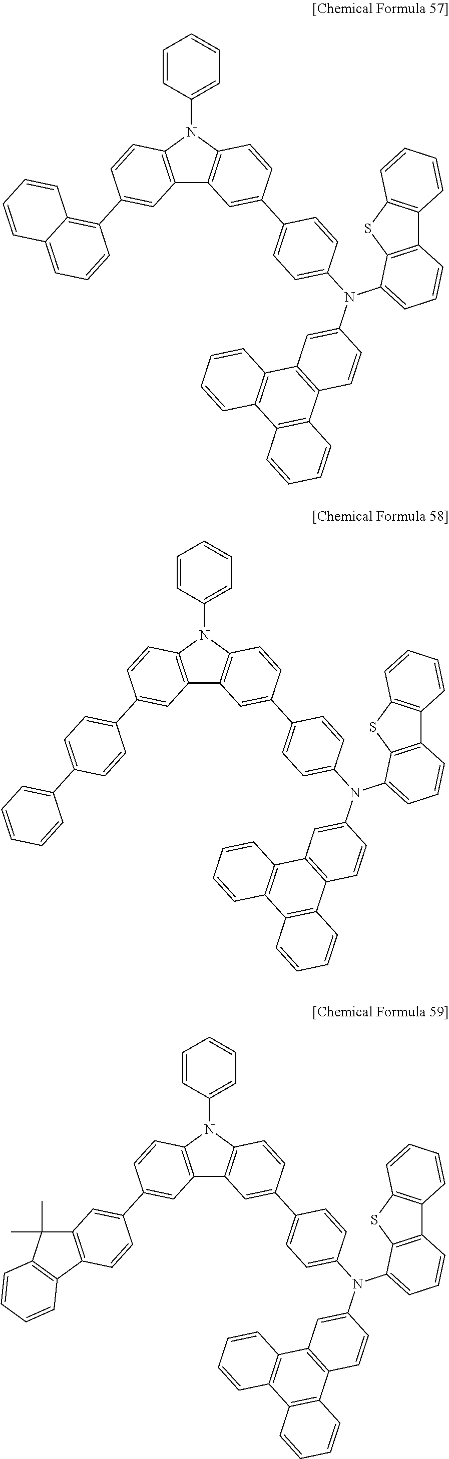 [SMILES: c1ccc(-n2c3ccc(-c4ccc(N(c5ccc6c7ccccc7c7ccccc7c6c5)c5cccc6c5sc5ccccc56)cc4)cc3c3cc(-c4cccc5ccccc45)ccc32)cc1 ; c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc21]

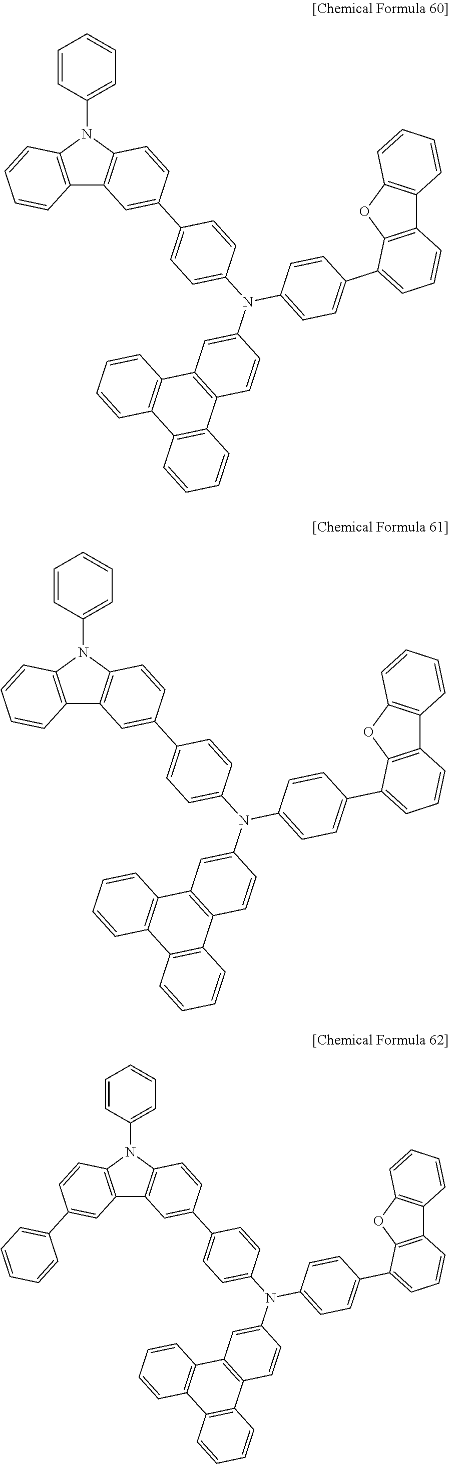 [SMILES: c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1 ; c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1 ; c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1]

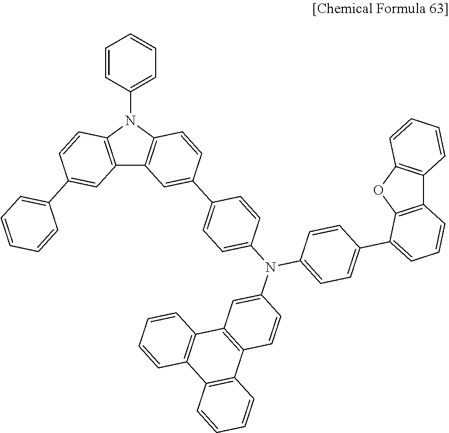 [SMILES: c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1]

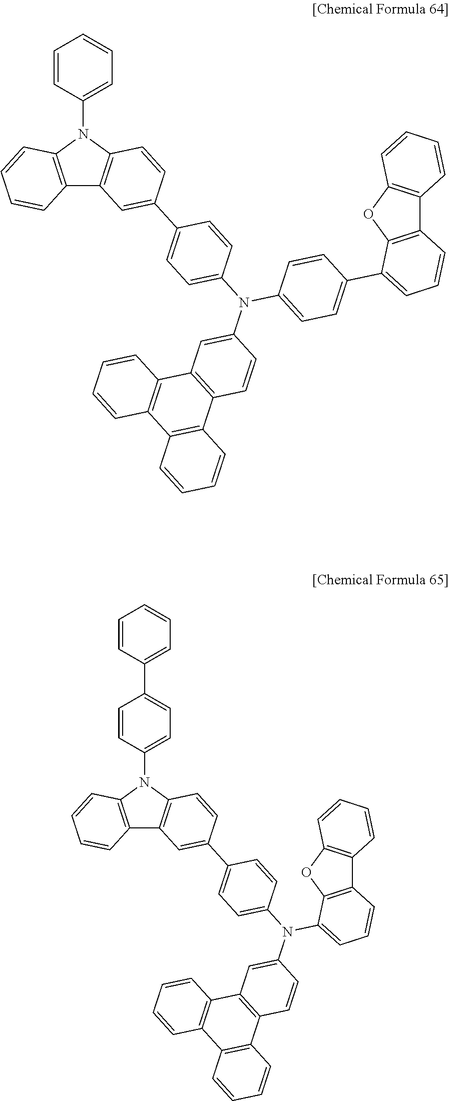 [SMILES: c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1 ; c1ccc(-c2ccc(-n3c4ccccc4c4cc(-c5ccc(N(c6ccc7c8ccccc8c8ccccc8c7c6)c6cccc7c6oc6ccccc67)cc5)ccc43)cc2)cc1]

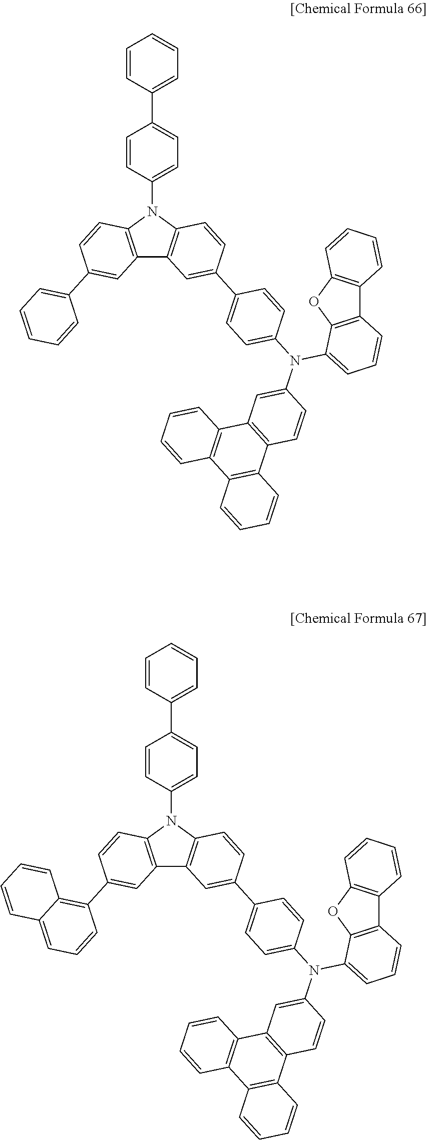 [SMILES: c1ccc(-c2ccc(-n3c4ccc(-c5ccccc5)cc4c4cc(-c5ccc(N(c6ccc7c8ccccc8c8ccccc8c7c6)c6cccc7c6oc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6oc6ccccc67)cc5)cc4c4cc(-c5cccc6ccccc56)ccc43)cc2)cc1]

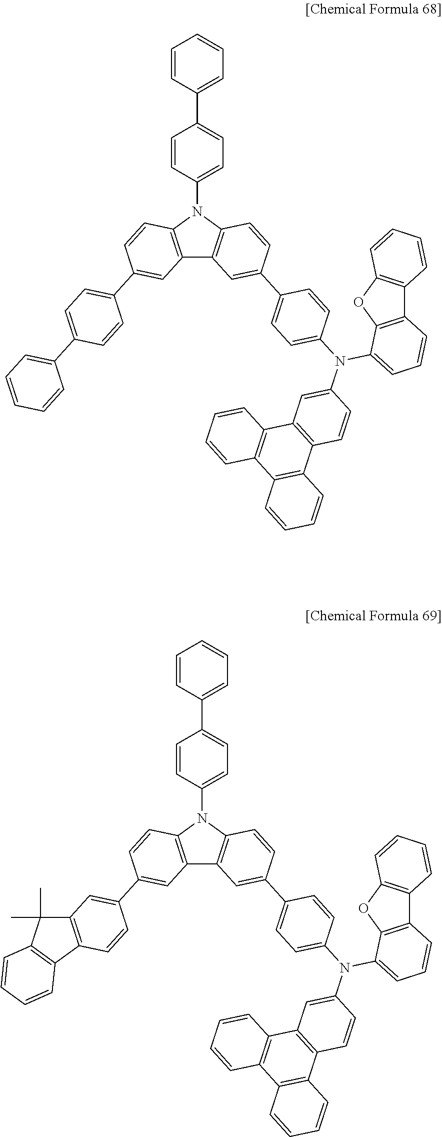 [SMILES: c1ccc(-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc21]

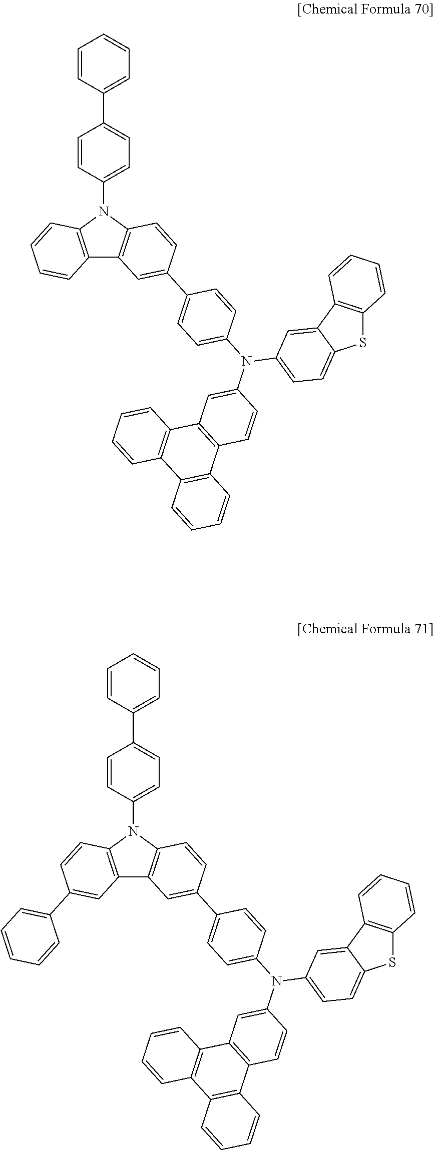 [SMILES: c1ccc(-c2ccc(-n3c4ccccc4c4cc(-c5ccc(N(c6ccc7sc8ccccc8c7c6)c6ccc7c8ccccc8c8ccccc8c7c6)cc5)ccc43)cc2)cc1 ; c1ccc(-c2ccc(-n3c4ccc(-c5ccccc5)cc4c4cc(-c5ccc(N(c6ccc7sc8ccccc8c7c6)c6ccc7c8ccccc8c8ccccc8c7c6)cc5)ccc43)cc2)cc1]

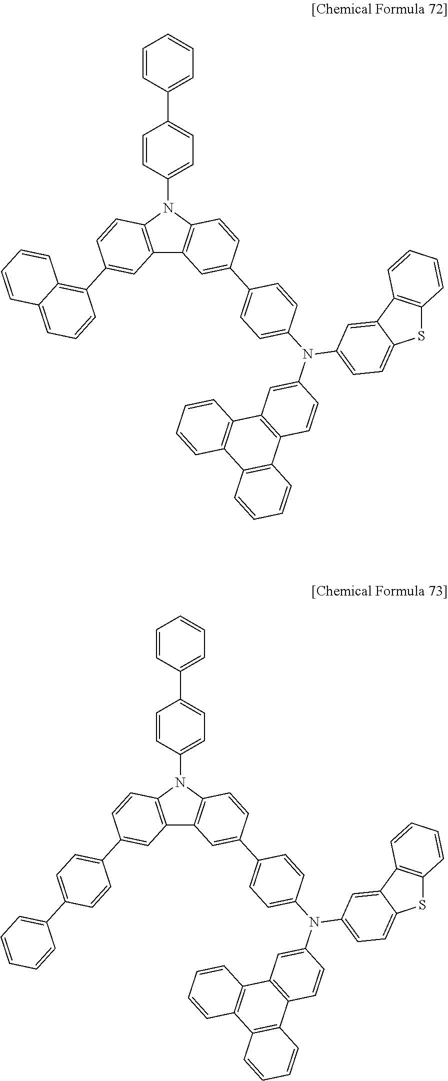 [SMILES: c1ccc(-c2ccc(-n3c4ccc(-c5ccc(N(c6ccc7sc8ccccc8c7c6)c6ccc7c8ccccc8c8ccccc8c7c6)cc5)cc4c4cc(-c5cccc6ccccc56)ccc43)cc2)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc2)cc1]

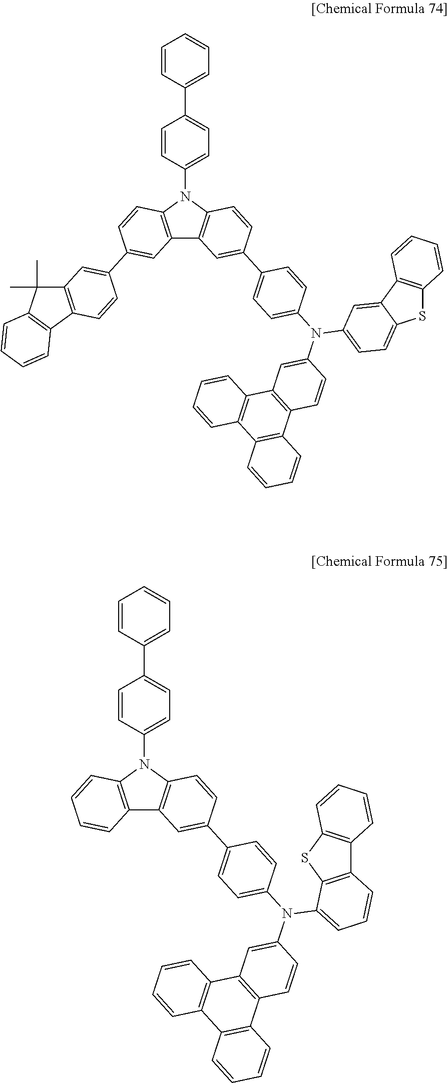 [SMILES: CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc21 ; c1ccc(-c2ccc(-n3c4ccccc4c4cc(-c5ccc(N(c6ccc7c8ccccc8c8ccccc8c7c6)c6cccc7c6sc6ccccc67)cc5)ccc43)cc2)cc1]

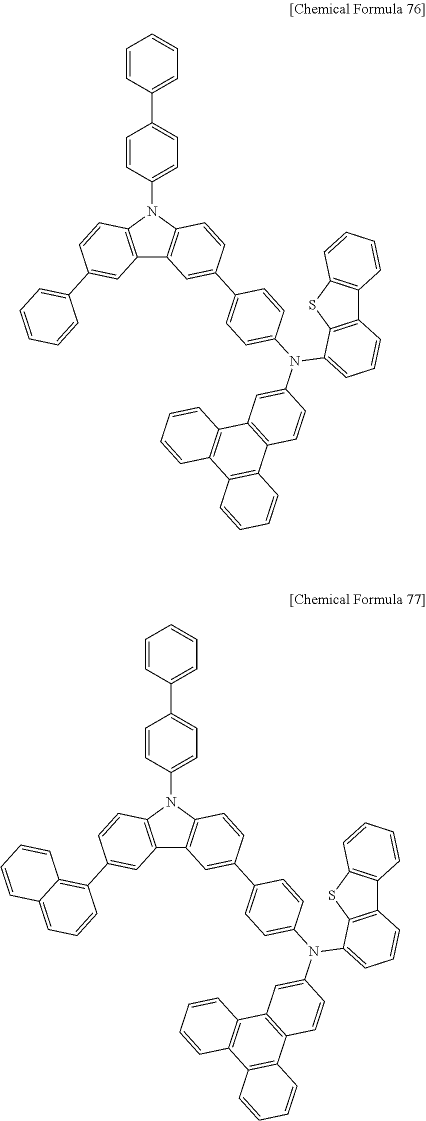 [SMILES: c1ccc(-c2ccc(-n3c4ccc(-c5ccccc5)cc4c4cc(-c5ccc(N(c6ccc7c8ccccc8c8ccccc8c7c6)c6cccc7c6sc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6sc6ccccc67)cc5)cc4c4cc(-c5cccc6ccccc56)ccc43)cc2)cc1]

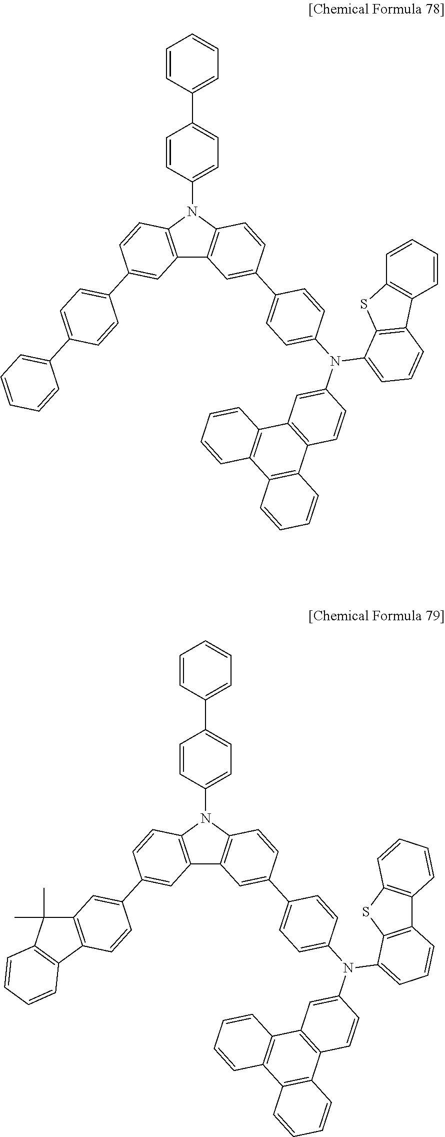 [SMILES: c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc21]

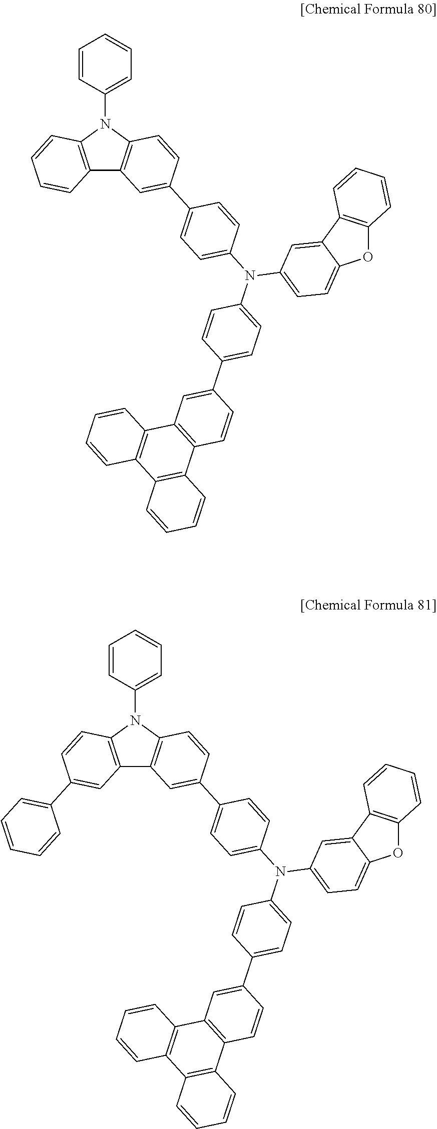 [SMILES: c1ccc(-n2c3ccccc3c3cc(-c4ccc(N(c5ccc(-c6ccc7c8ccccc8c8ccccc8c7c6)cc5)c5ccc6oc7ccccc7c6c5)cc4)ccc32)cc1 ; c1ccc(-c2ccc3c(c2)c2cc(-c4ccc(N(c5ccc(-c6ccc7c8ccccc8c8ccccc8c7c6)cc5)c5ccc6oc7ccccc7c6c5)cc4)ccc2n3-c2ccccc2)cc1]

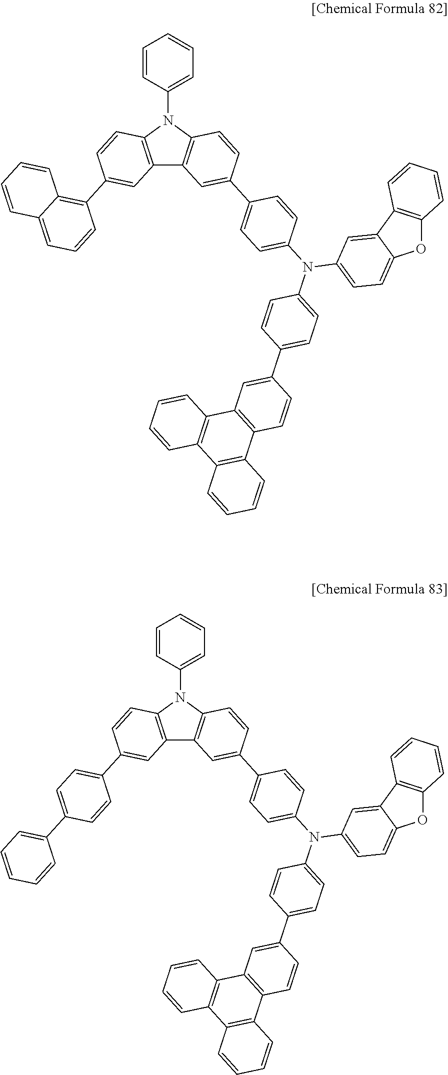 [SMILES: c1ccc(-n2c3ccc(-c4ccc(N(c5ccc(-c6ccc7c8ccccc8c8ccccc8c7c6)cc5)c5ccc6oc7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6ccc7oc8ccccc8c7c6)cc5)ccc3n4-c3ccccc3)cc2)cc1]

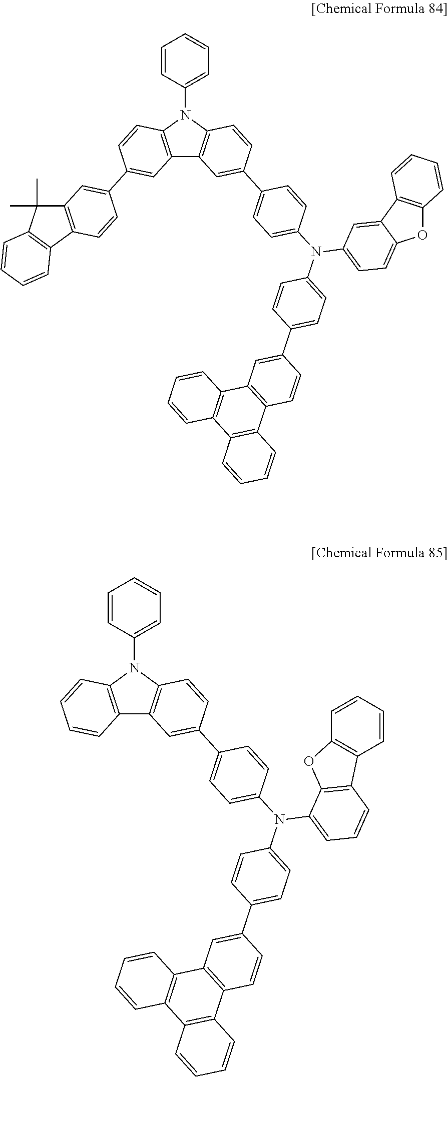

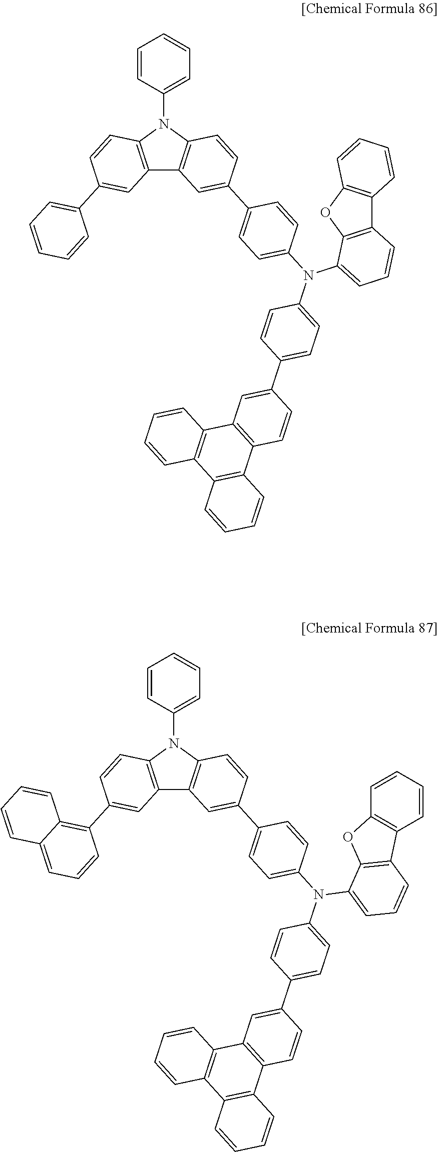 [SMILES: c1ccc(-c2ccc3c(c2)c2cc(-c4ccc(N(c5ccc(-c6ccc7c8ccccc8c8ccccc8c7c6)cc5)c5cccc6c5oc5ccccc56)cc4)ccc2n3-c2ccccc2)cc1 ; c1ccc(-n2c3ccc(-c4ccc(N(c5ccc(-c6ccc7c8ccccc8c8ccccc8c7c6)cc5)c5cccc6c5oc5ccccc56)cc4)cc3c3cc(-c4cccc5ccccc45)ccc32)cc1]

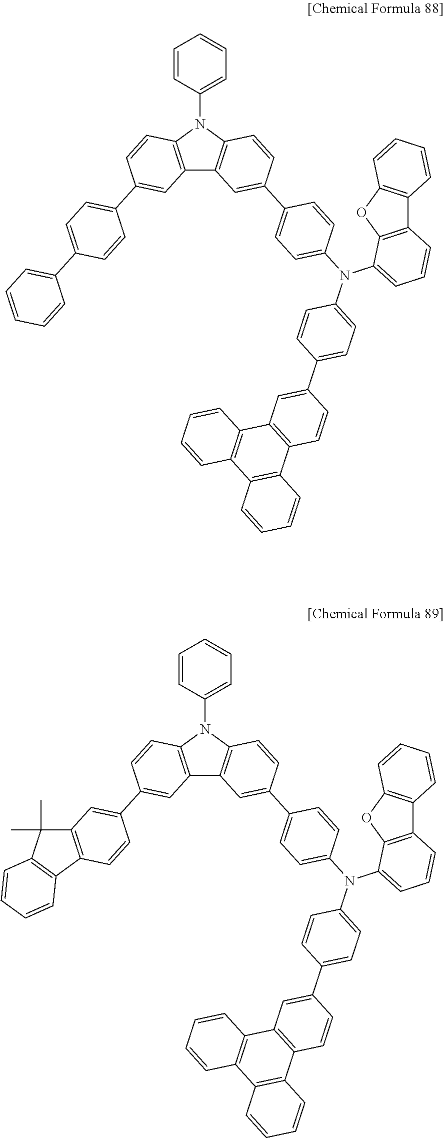 [SMILES: c1ccc(-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc21]

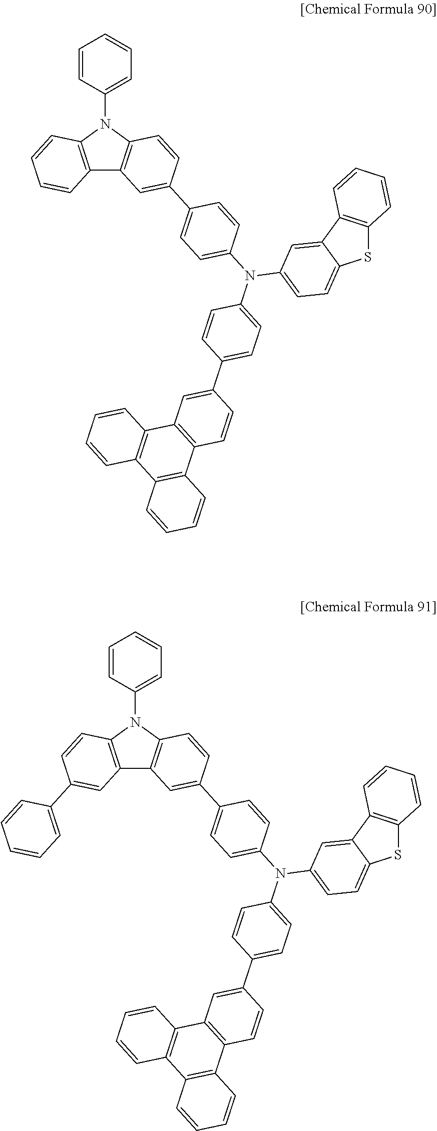 [SMILES: c1ccc(-n2c3ccccc3c3cc(-c4ccc(N(c5ccc(-c6ccc7c8ccccc8c8ccccc8c7c6)cc5)c5ccc6sc7ccccc7c6c5)cc4)ccc32)cc1 ; c1ccc(-c2ccc3c(c2)c2cc(-c4ccc(N(c5ccc(-c6ccc7c8ccccc8c8ccccc8c7c6)cc5)c5ccc6sc7ccccc7c6c5)cc4)ccc2n3-c2ccccc2)cc1]

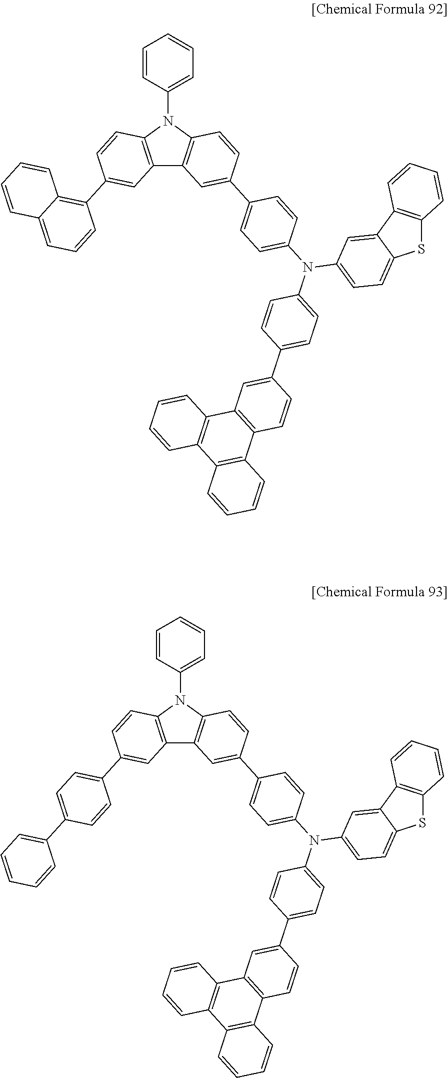 [SMILES: c1ccc(-n2c3ccc(-c4ccc(N(c5ccc(-c6ccc7c8ccccc8c8ccccc8c7c6)cc5)c5ccc6sc7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc2)cc1]

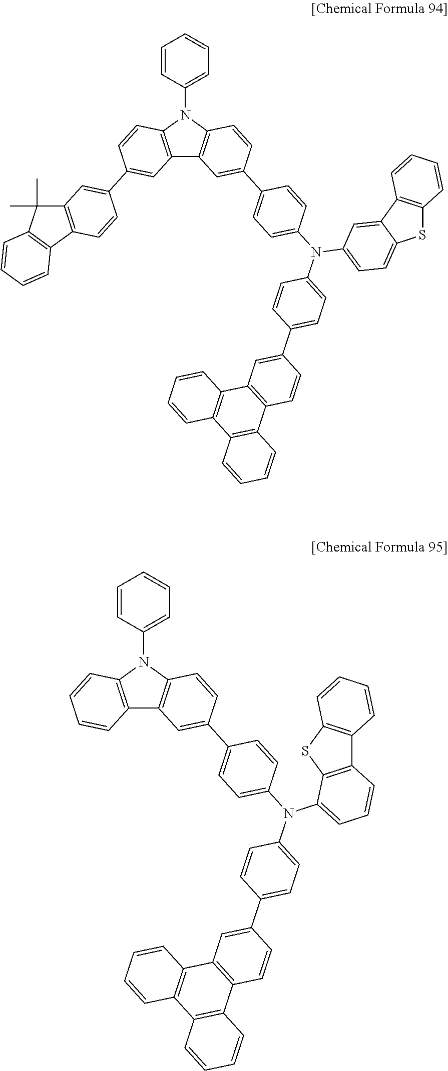 [SMILES: CC1(C)c2ccccc2-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc21 ; c1ccc(-n2c3ccccc3c3cc(-c4ccc(N(c5ccc(-c6ccc7c8ccccc8c8ccccc8c7c6)cc5)c5cccc6c5sc5ccccc56)cc4)ccc32)cc1]

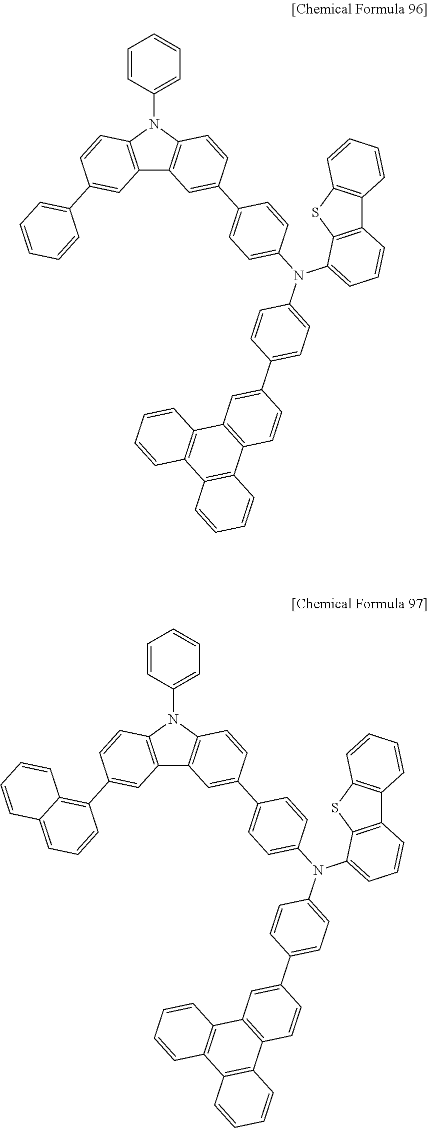 [SMILES: c1ccc(-c2ccc3c(c2)c2cc(-c4ccc(N(c5ccc(-c6ccc7c8ccccc8c8ccccc8c7c6)cc5)c5cccc6c5sc5ccccc56)cc4)ccc2n3-c2ccccc2)cc1 ; c1ccc(-n2c3ccc(-c4ccc(N(c5ccc(-c6ccc7c8ccccc8c8ccccc8c7c6)cc5)c5cccc6c5sc5ccccc56)cc4)cc3c3cc(-c4cccc5ccccc45)ccc32)cc1]

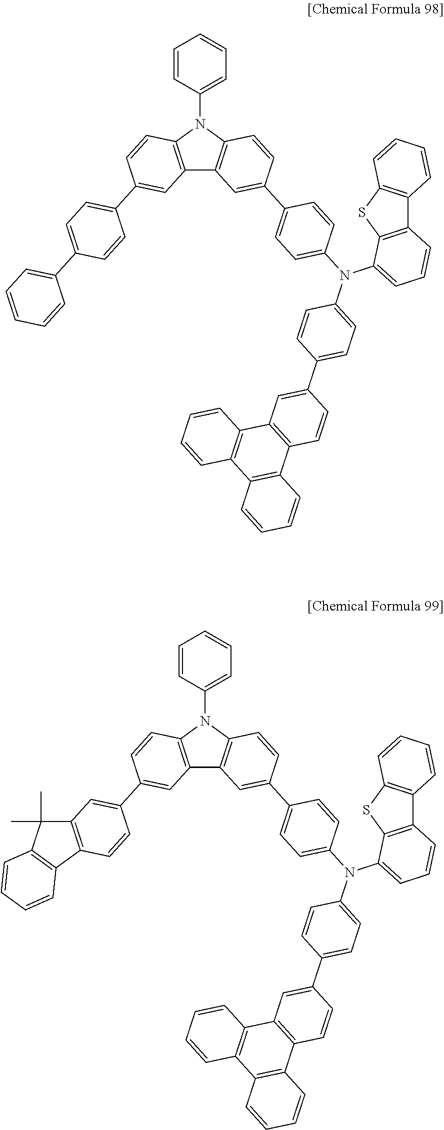 [SMILES: c1ccc(-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc21]

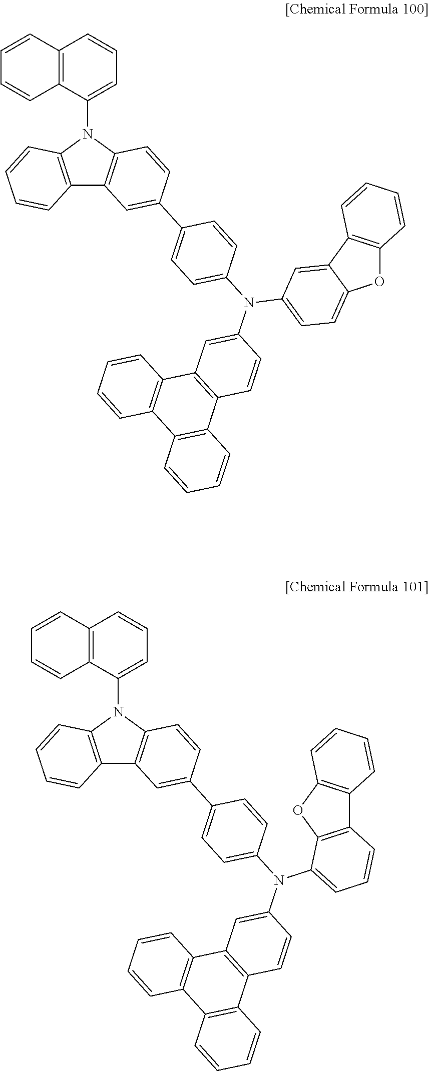 [SMILES: c1ccc2c(-n3c4ccccc4c4cc(-c5ccc(N(c6ccc7oc8ccccc8c7c6)c6ccc7c8ccccc8c8ccccc8c7c6)cc5)ccc43)cccc2c1 ; c1ccc2c(-n3c4ccccc4c4cc(-c5ccc(N(c6ccc7c8ccccc8c8ccccc8c7c6)c6cccc7c6oc6ccccc67)cc5)ccc43)cccc2c1]

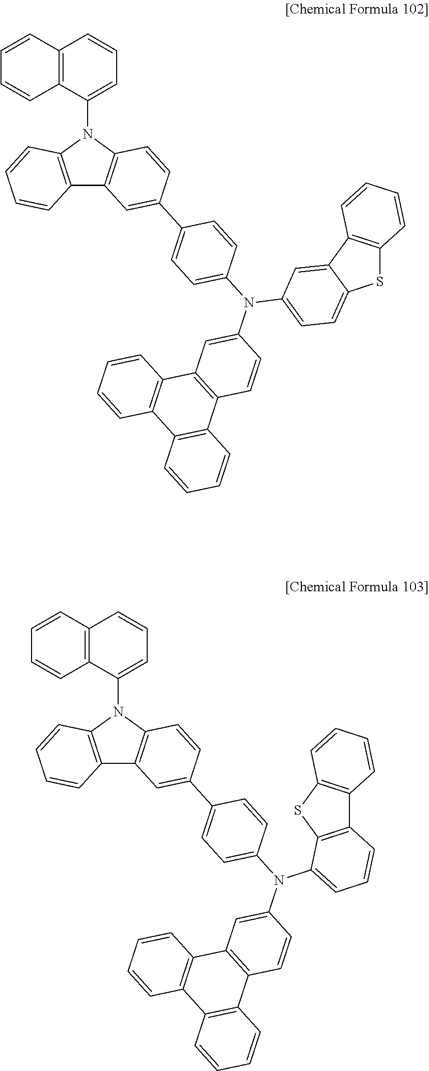 [SMILES: c1ccc2c(-n3c4ccccc4c4cc(-c5ccc(N(c6ccc7sc8ccccc8c7c6)c6ccc7c8ccccc8c8ccccc8c7c6)cc5)ccc43)cccc2c1 ; c1ccc2c(-n3c4ccccc4c4cc(-c5ccc(N(c6ccc7c8ccccc8c8ccccc8c7c6)c6cccc7c6sc6ccccc67)cc5)ccc43)cccc2c1]

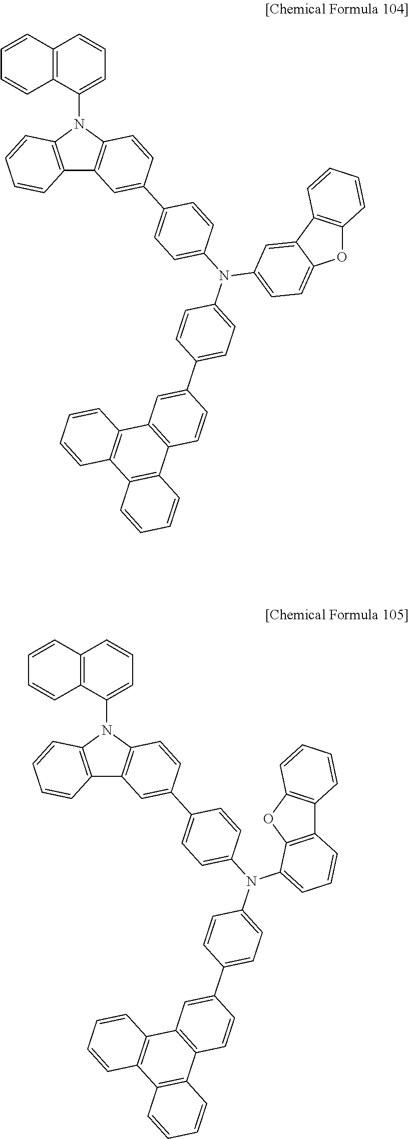 [SMILES: c1ccc2c(-n3c4ccccc4c4cc(-c5ccc(N(c6ccc(-c7ccc8c9ccccc9c9ccccc9c8c7)cc6)c6ccc7oc8ccccc8c7c6)cc5)ccc43)cccc2c1 ; c1ccc2c(-n3c4ccccc4c4cc(-c5ccc(N(c6ccc(-c7ccc8c9ccccc9c9ccccc9c8c7)cc6)c6cccc7c6oc6ccccc67)cc5)ccc43)cccc2c1]

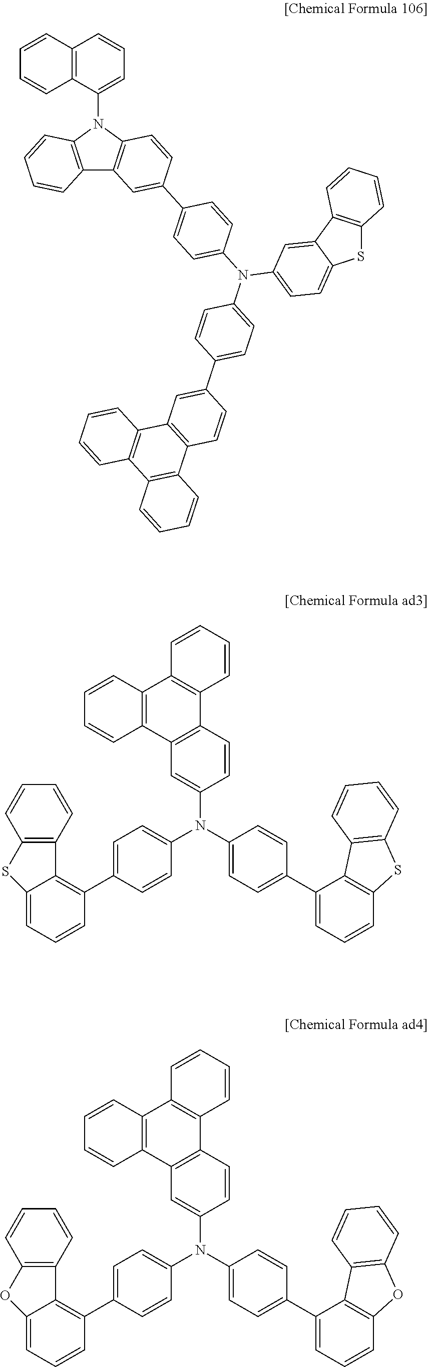 [SMILES: c1ccc2c(-n3c4ccccc4c4cc(-c5ccc(N(c6ccc(-c7ccc8c9ccccc9c9ccccc9c8c7)cc6)c6ccc7sc8ccccc8c7c6)cc5)ccc43)cccc2c1 ; c1ccc2c(c1)sc1cccc(-c3ccc(N(c4ccc(-c5cccc6sc7ccccc7c56)cc4)c4ccc5c6ccccc6c6ccccc6c5c4)cc3)c12 ; c1ccc2c(c1)oc1cccc(-c3ccc(N(c4ccc(-c5cccc6oc7ccccc7c56)cc4)c4ccc5c6ccccc6c6ccccc6c5c4)cc3)c12]

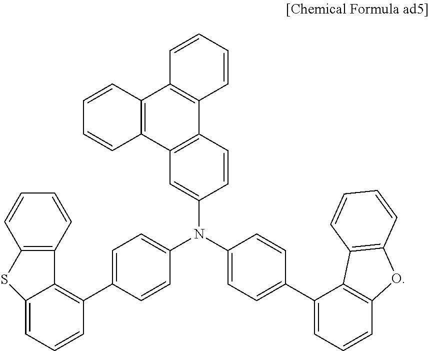 [SMILES: c1ccc2c(c1)oc1cccc(-c3ccc(N(c4ccc(-c5cccc6sc7ccccc7c56)cc4)c4ccc5c6ccccc6c6ccccc6c5c4)cc3)c12]

Embodiments are also directed to a compound for an organic photoelectric device, the compound being represented by one of the following Chemical Formulae 107 to 333:

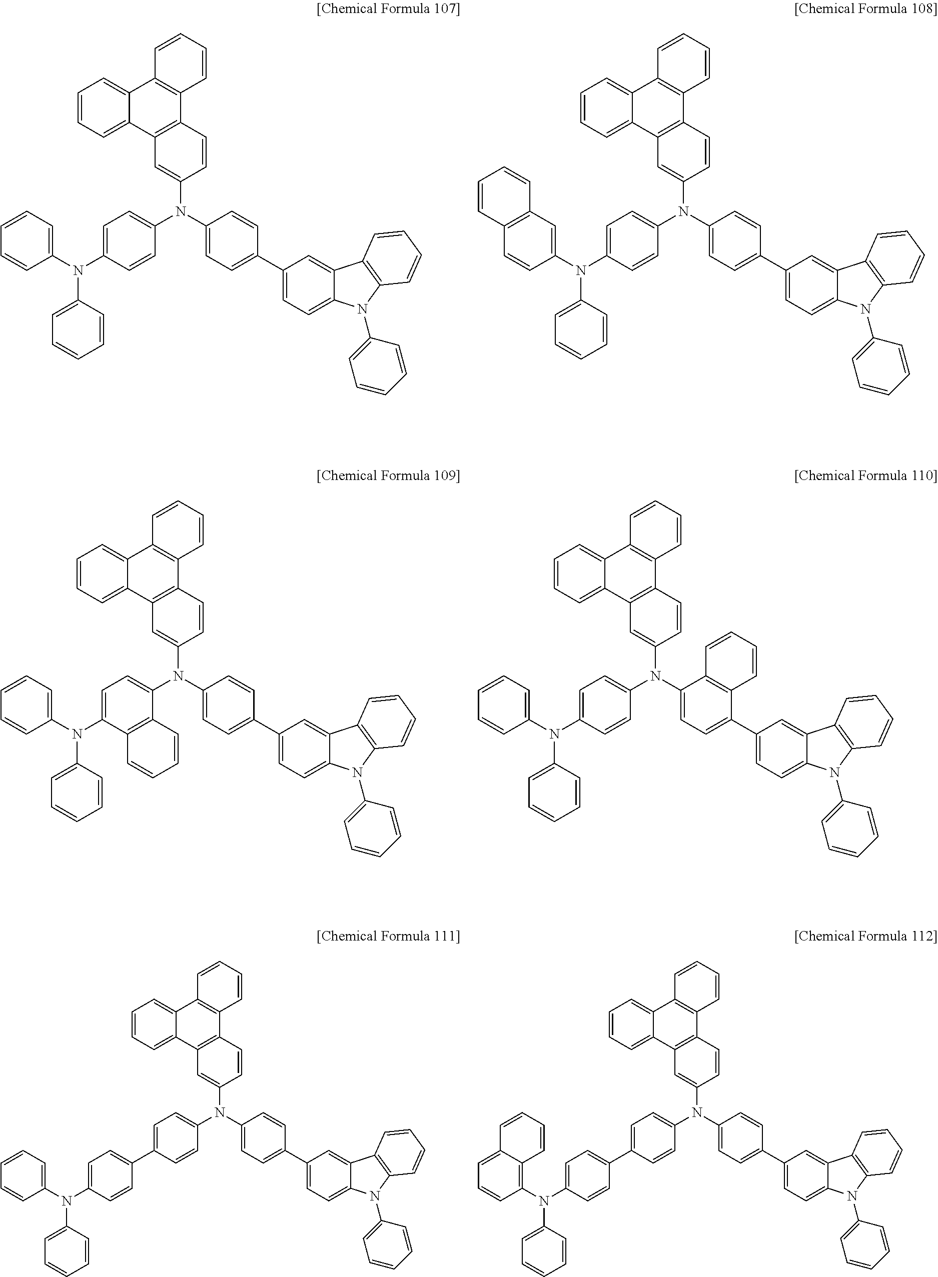

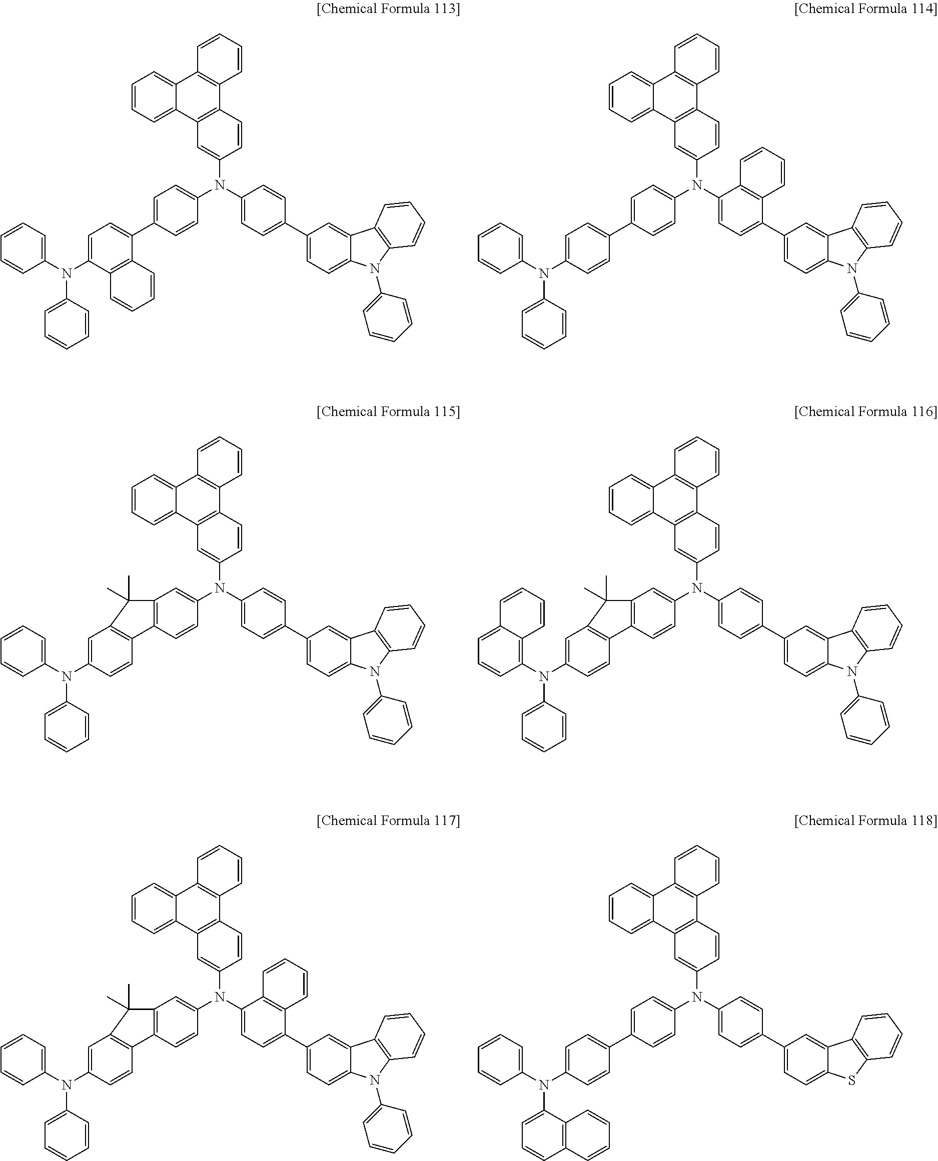

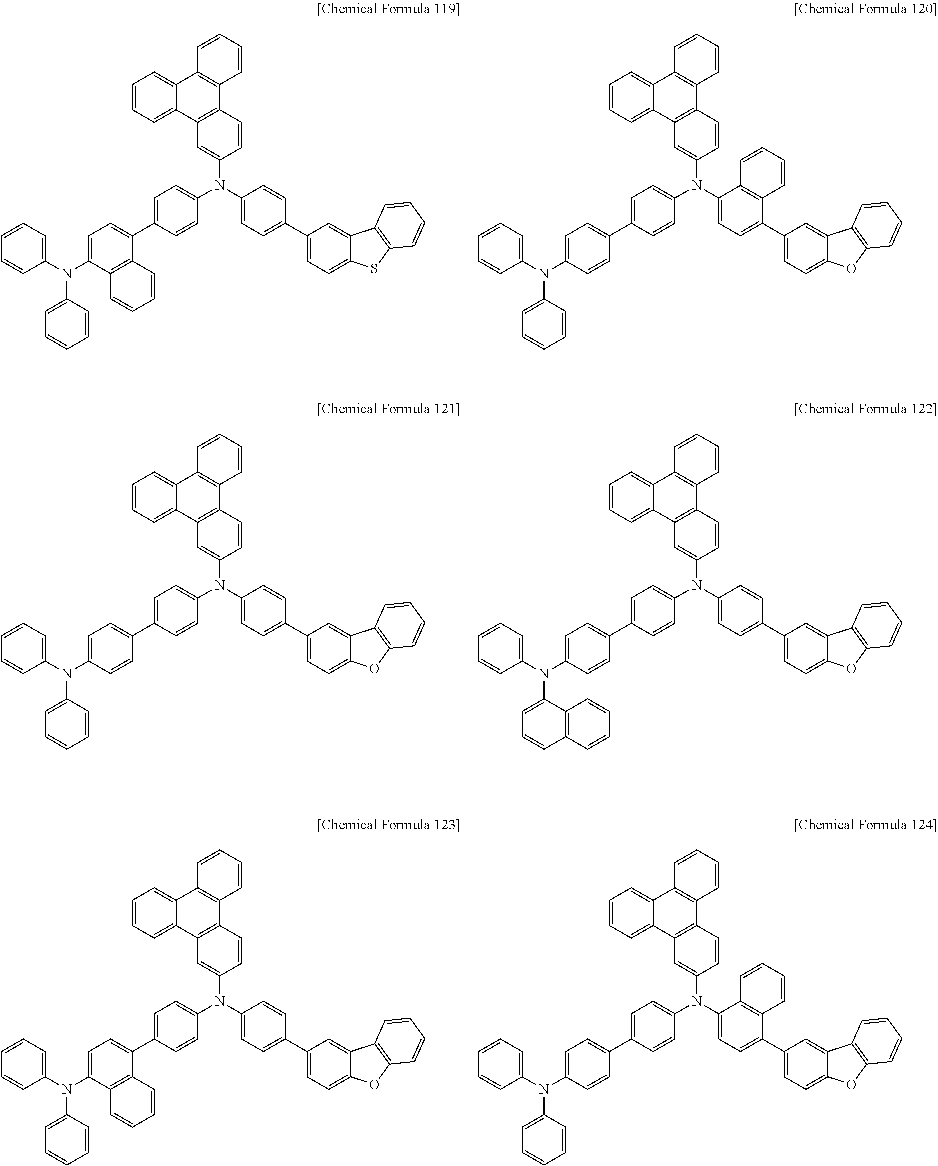

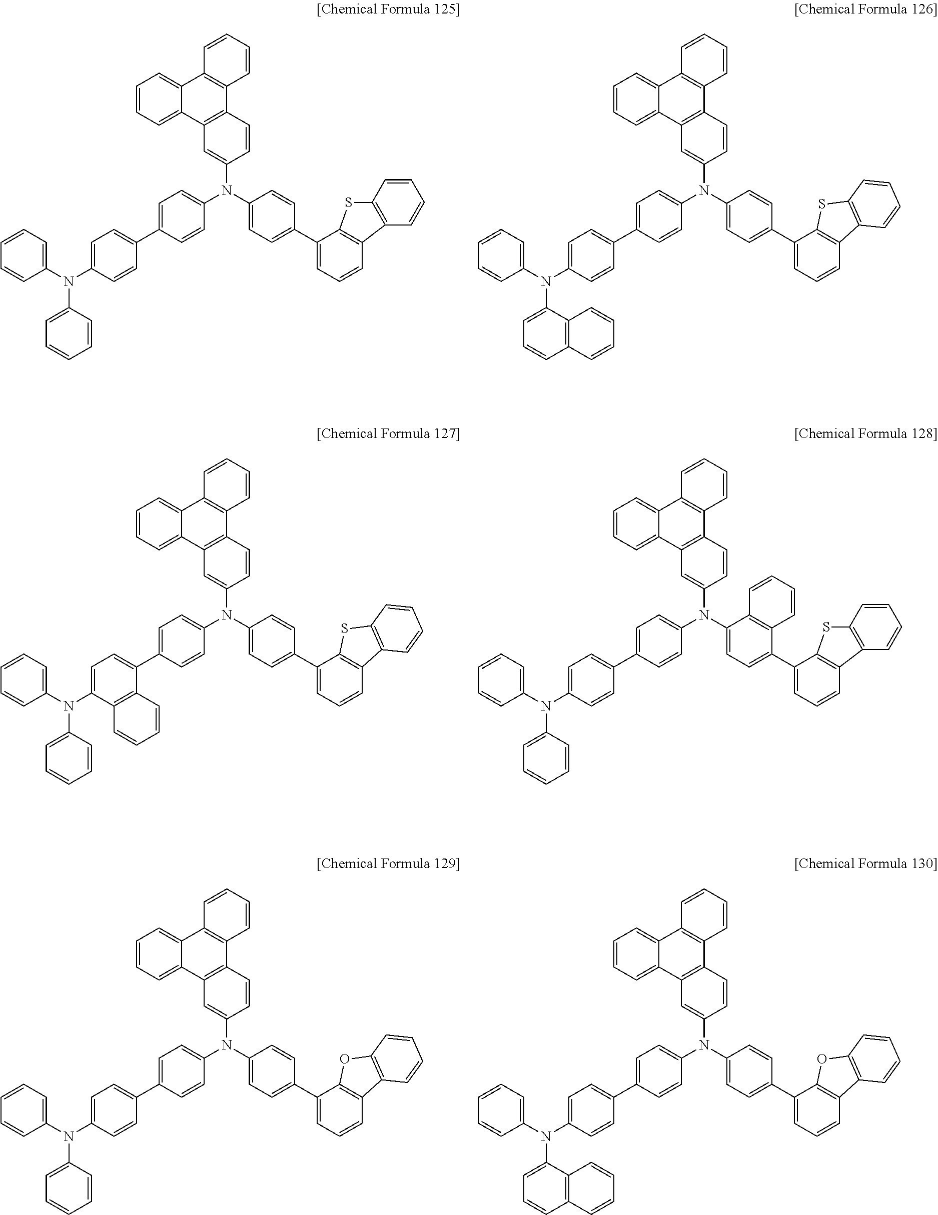

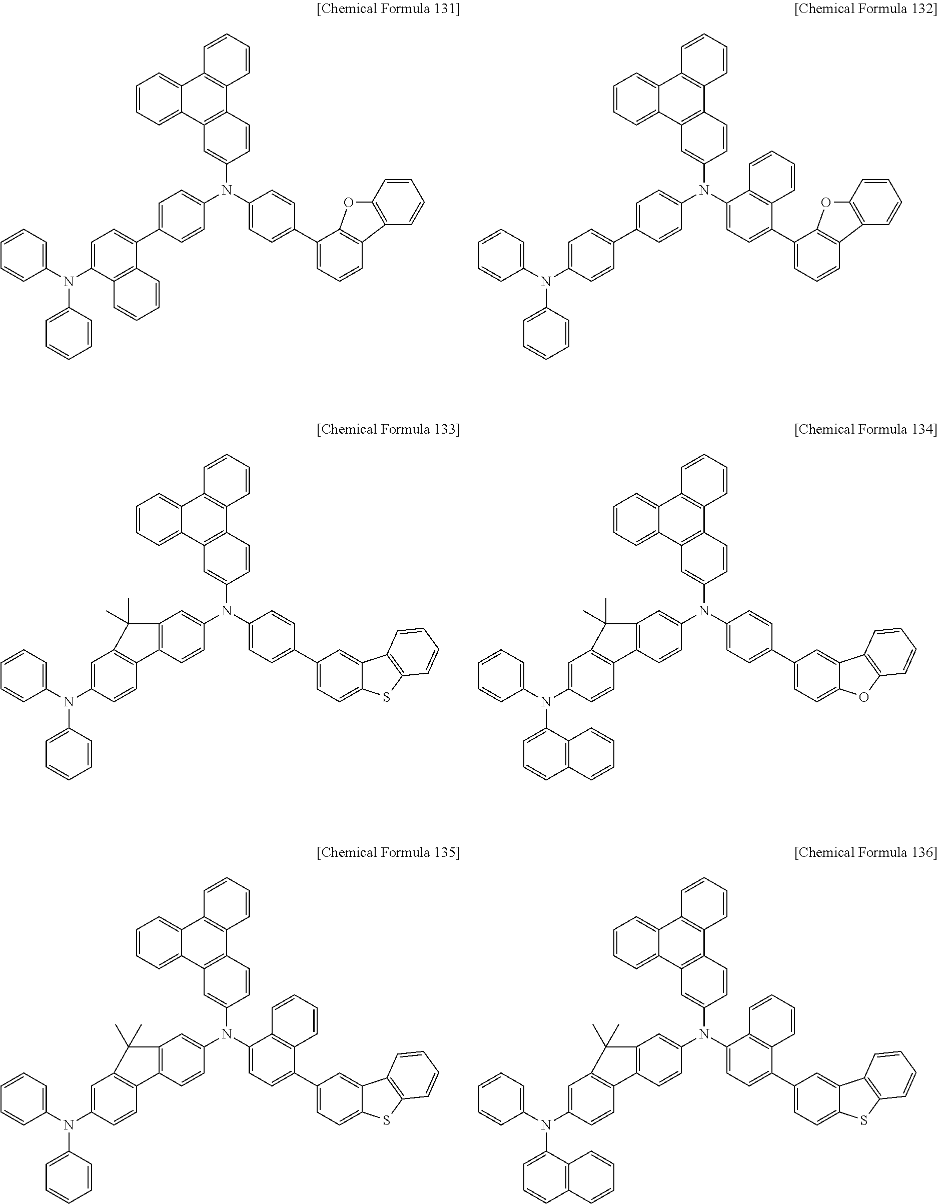

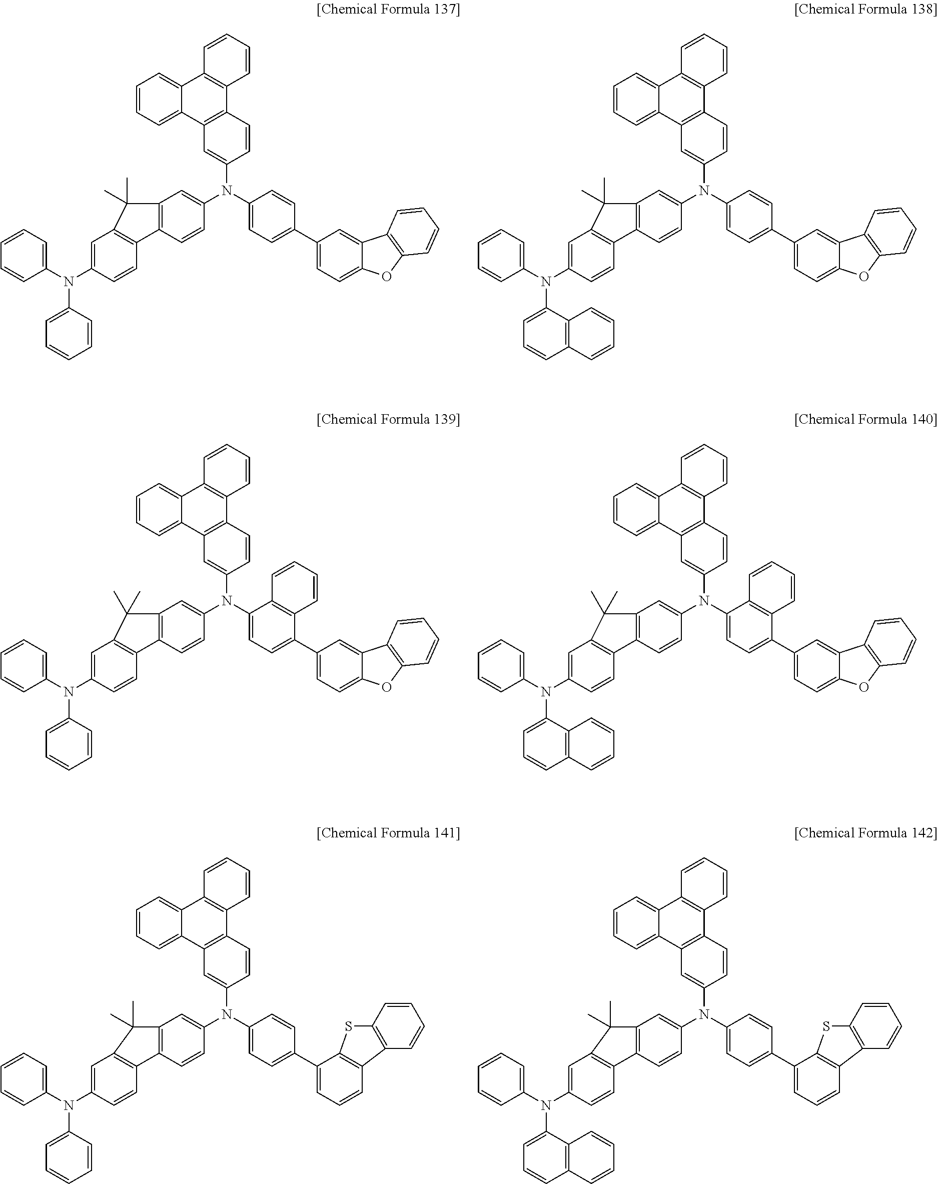

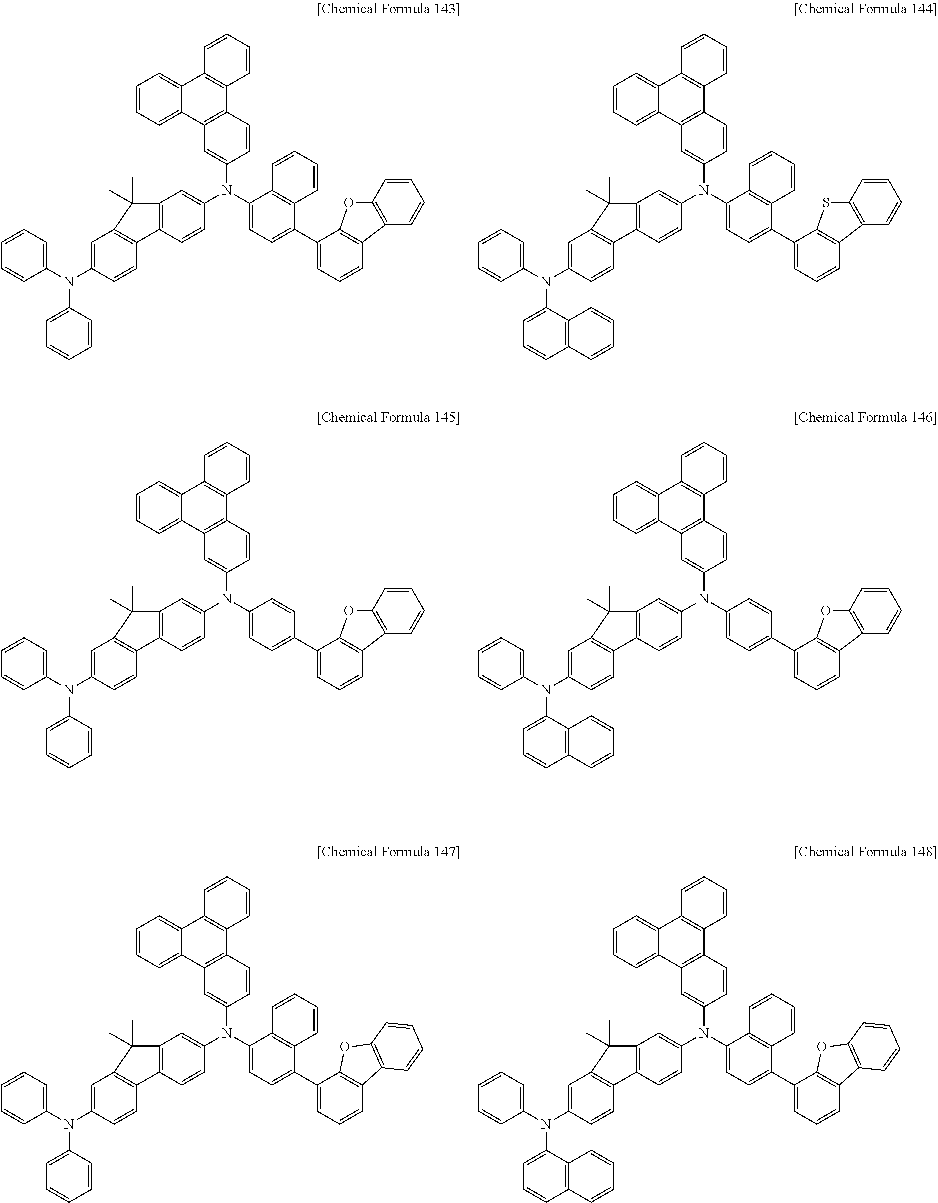

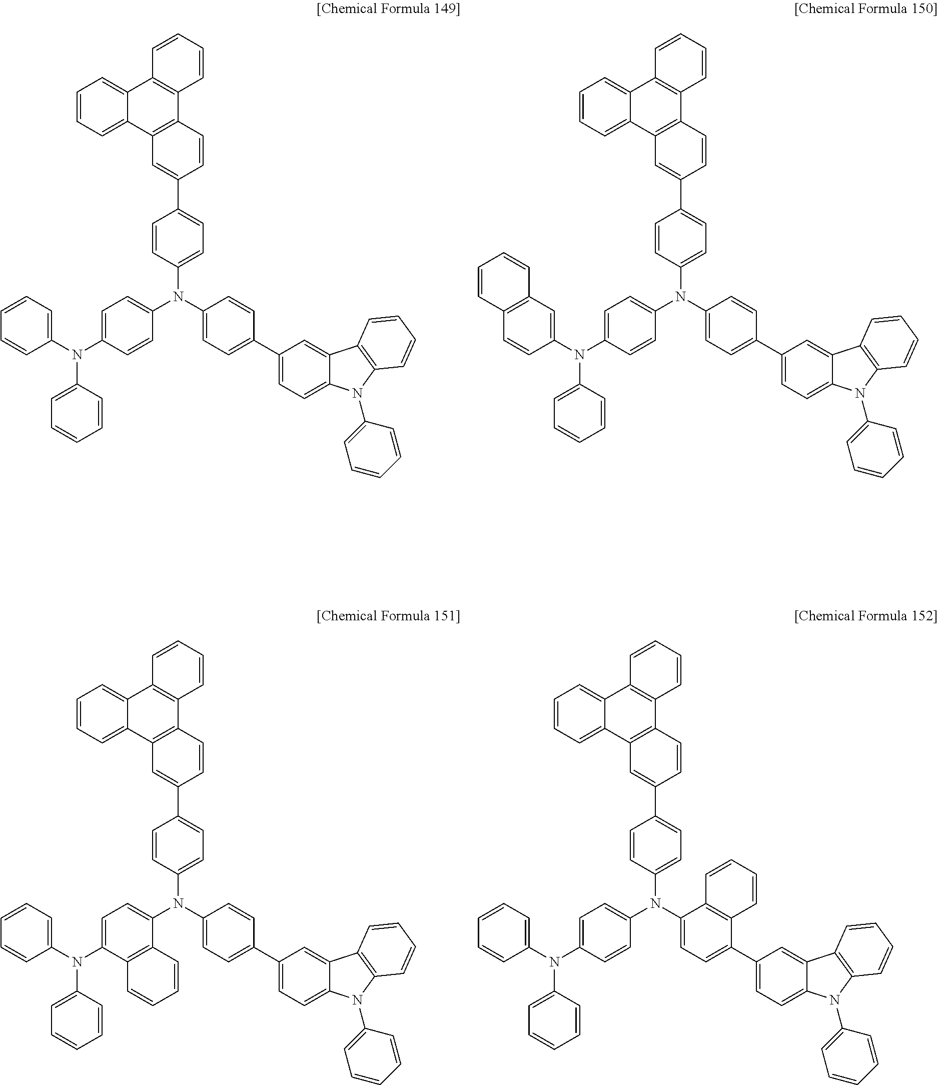 [SMILES: c1ccc(N(c2ccccc2)c2ccc(N(c3ccc(-c4ccc5c6ccccc6c6ccccc6c5c4)cc3)c3ccc(-c4ccc5c(c4)c4ccccc4n5-c4ccccc4)cc3)cc2)cc1 ; c1ccc(N(c2ccc(N(c3ccc(-c4ccc5c6ccccc6c6ccccc6c5c4)cc3)c3ccc(-c4ccc5c(c4)c4ccccc4n5-c4ccccc4)cc3)cc2)c2ccc3ccccc3c2)cc1 ; c1ccc(N(c2ccccc2)c2ccc(N(c3ccc(-c4ccc5c6ccccc6c6ccccc6c5c4)cc3)c3ccc(-c4ccc5c(c4)c4ccccc4n5-c4ccccc4)cc3)c3ccccc23)cc1 ; c1ccc(N(c2ccccc2)c2ccc(N(c3ccc(-c4ccc5c6ccccc6c6ccccc6c5c4)cc3)c3ccc(-c4ccc5c(c4)c4ccccc4n5-c4ccccc4)c4ccccc34)cc2)cc1]

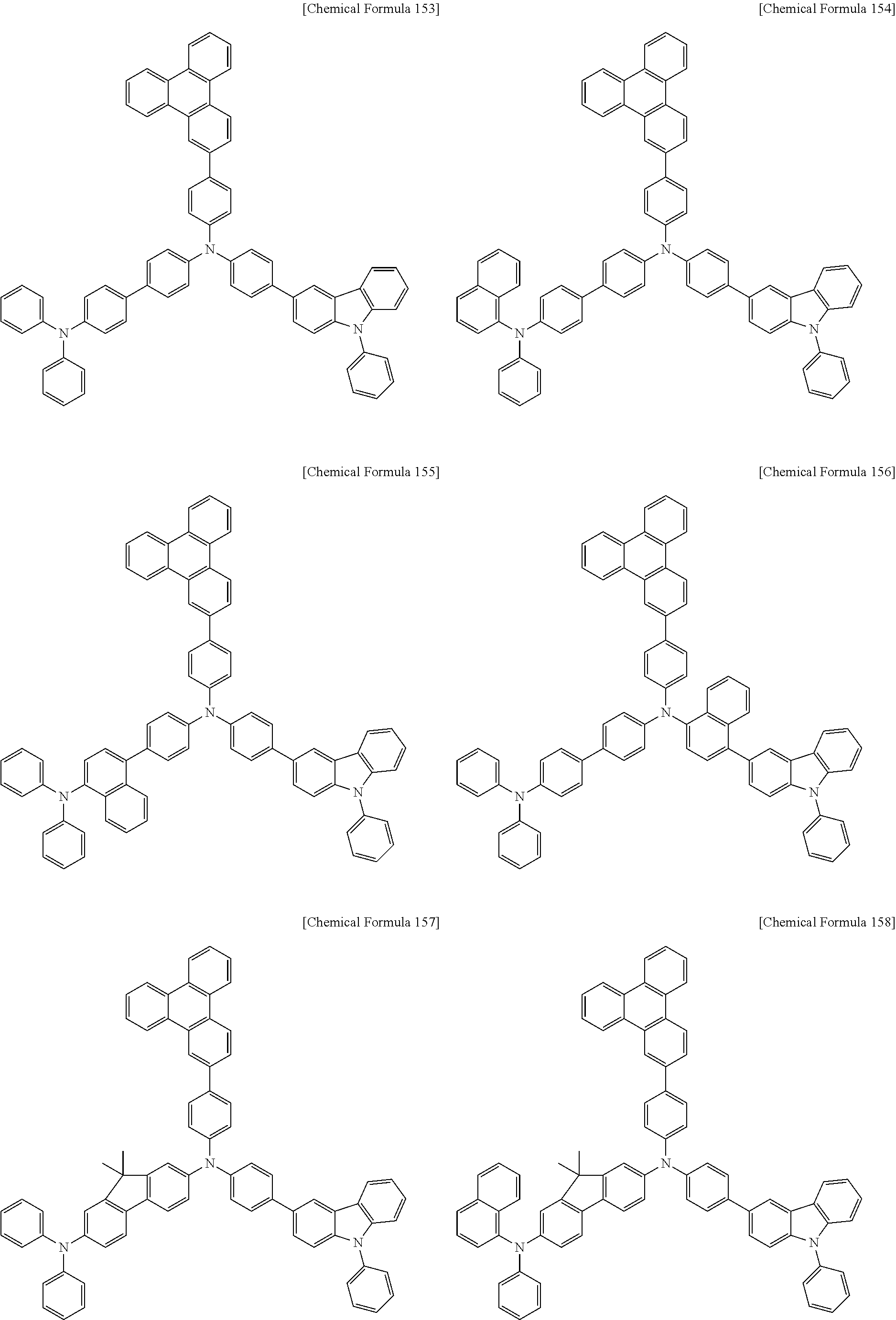

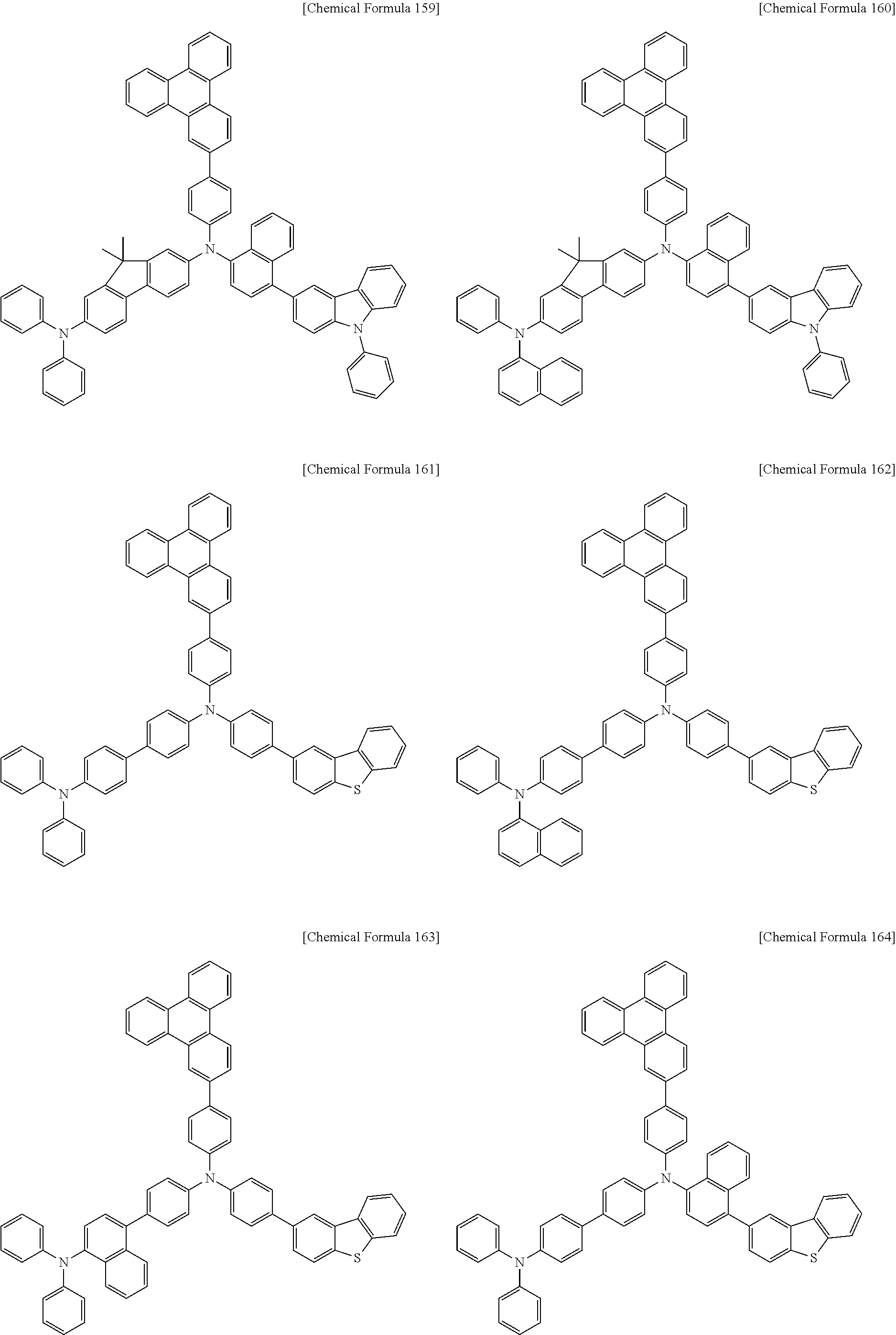

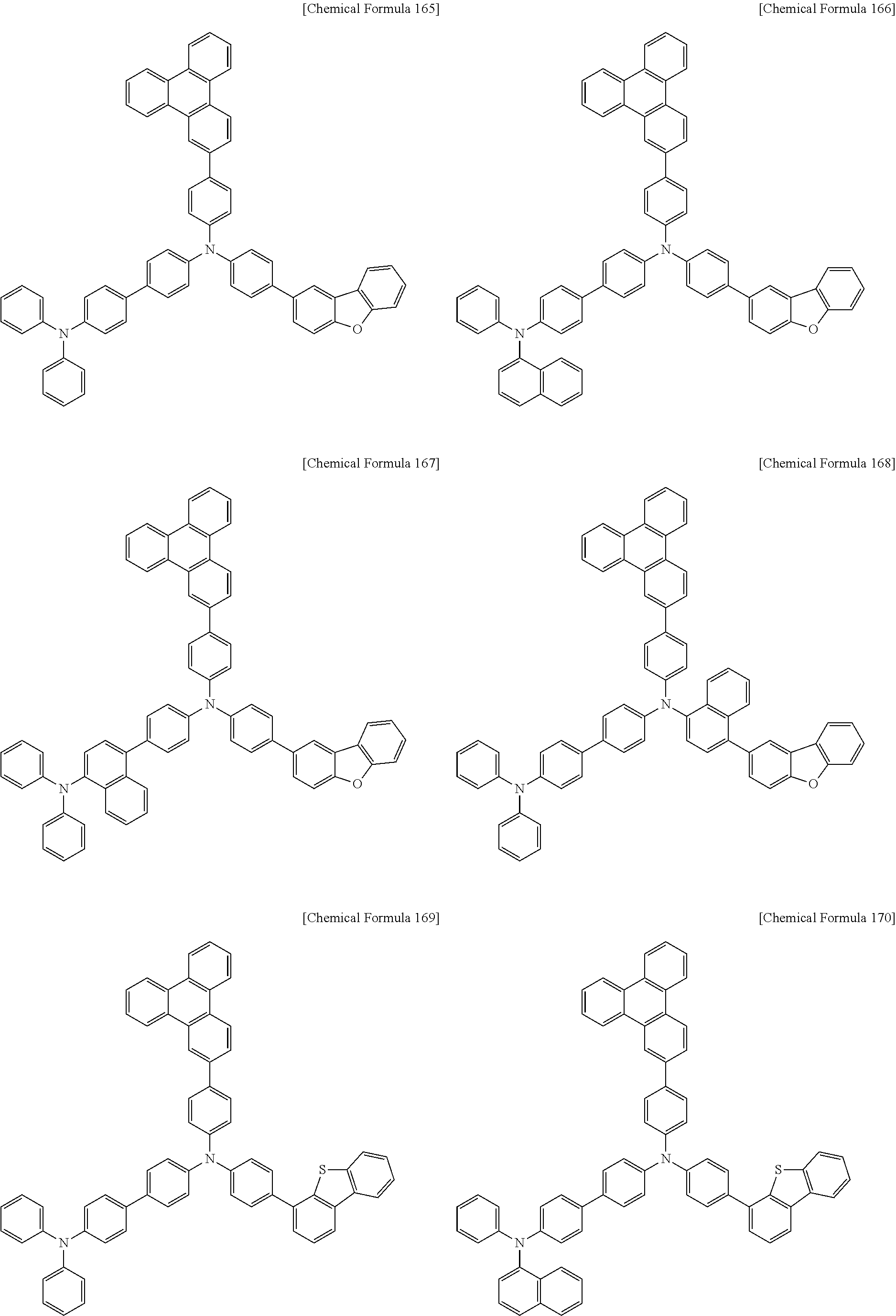

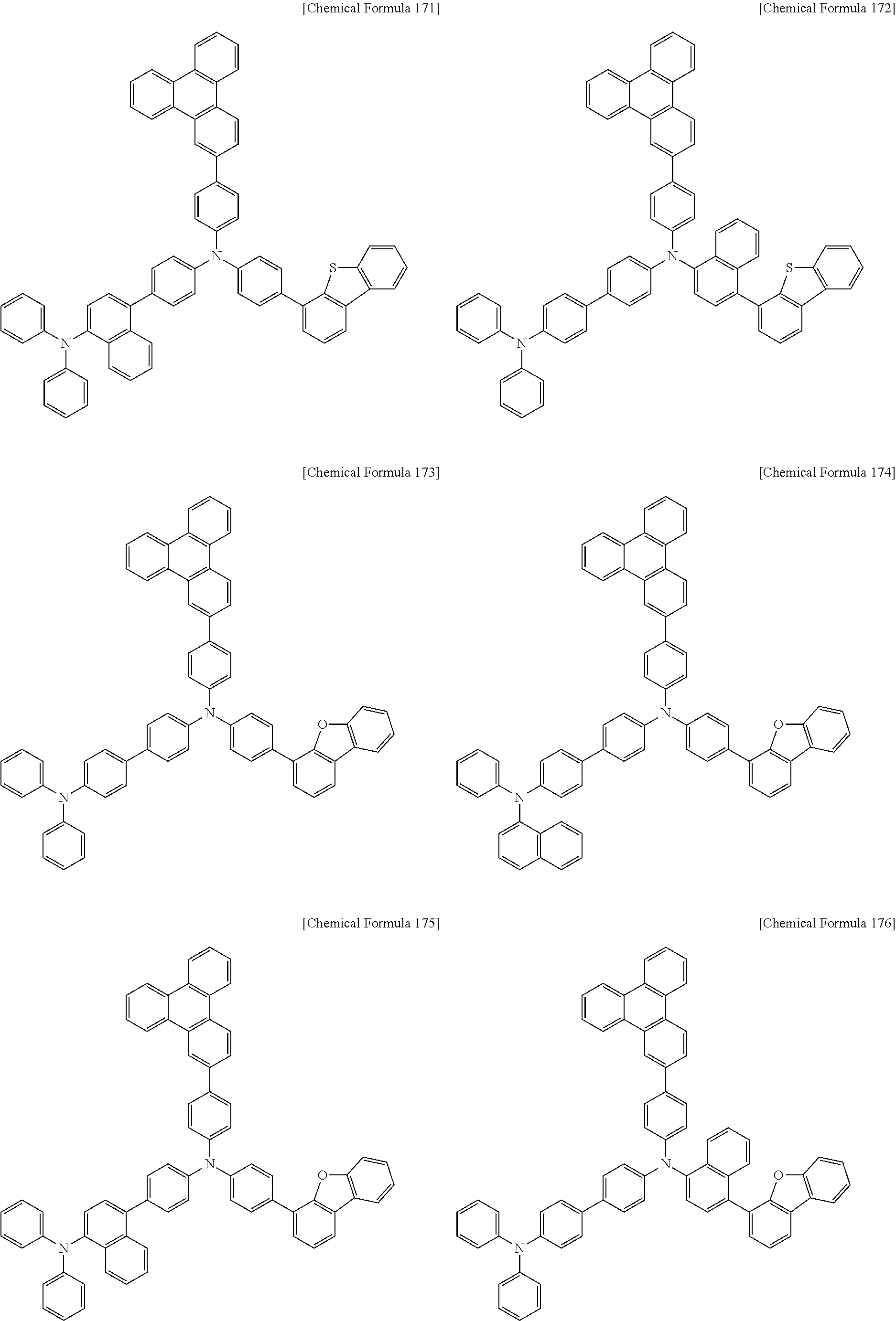

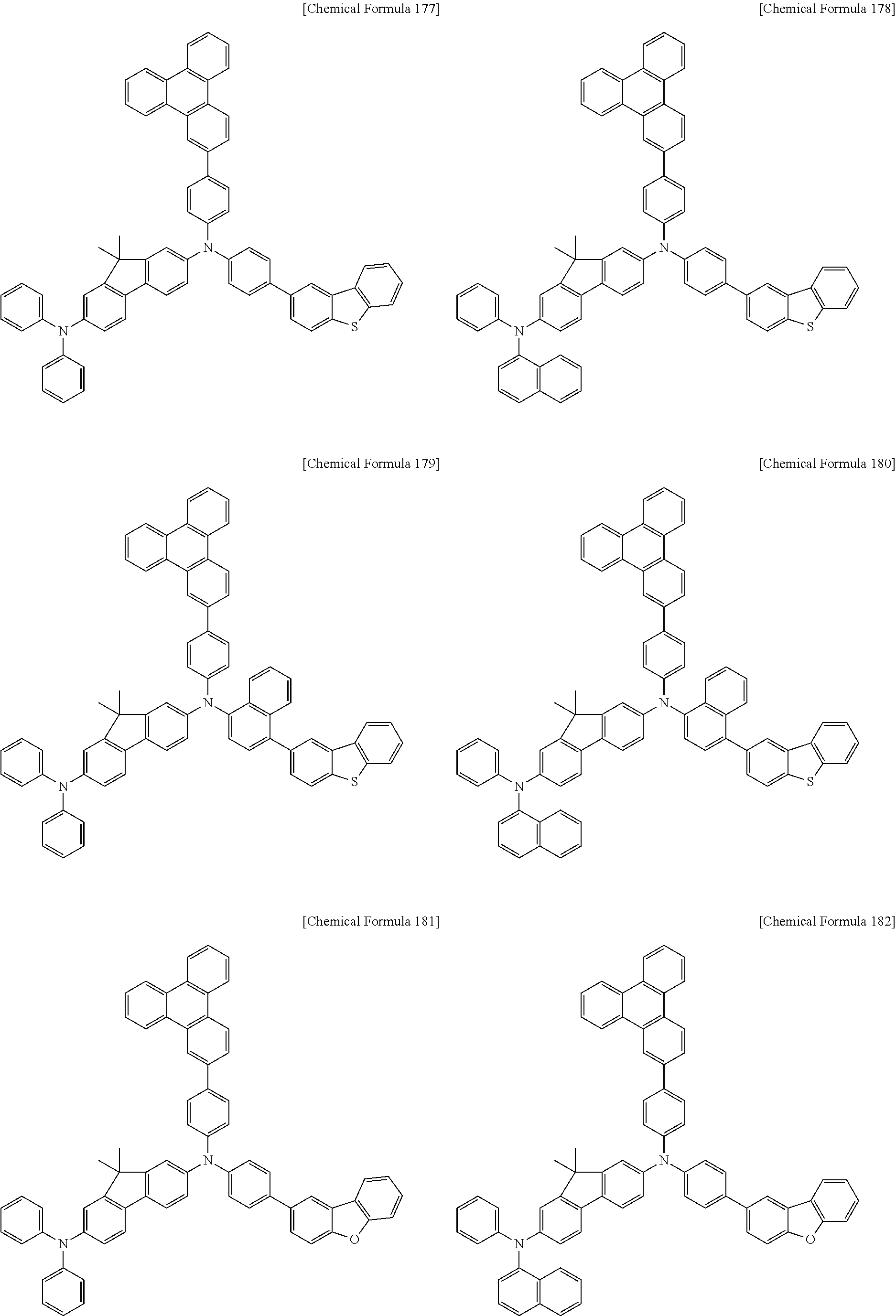

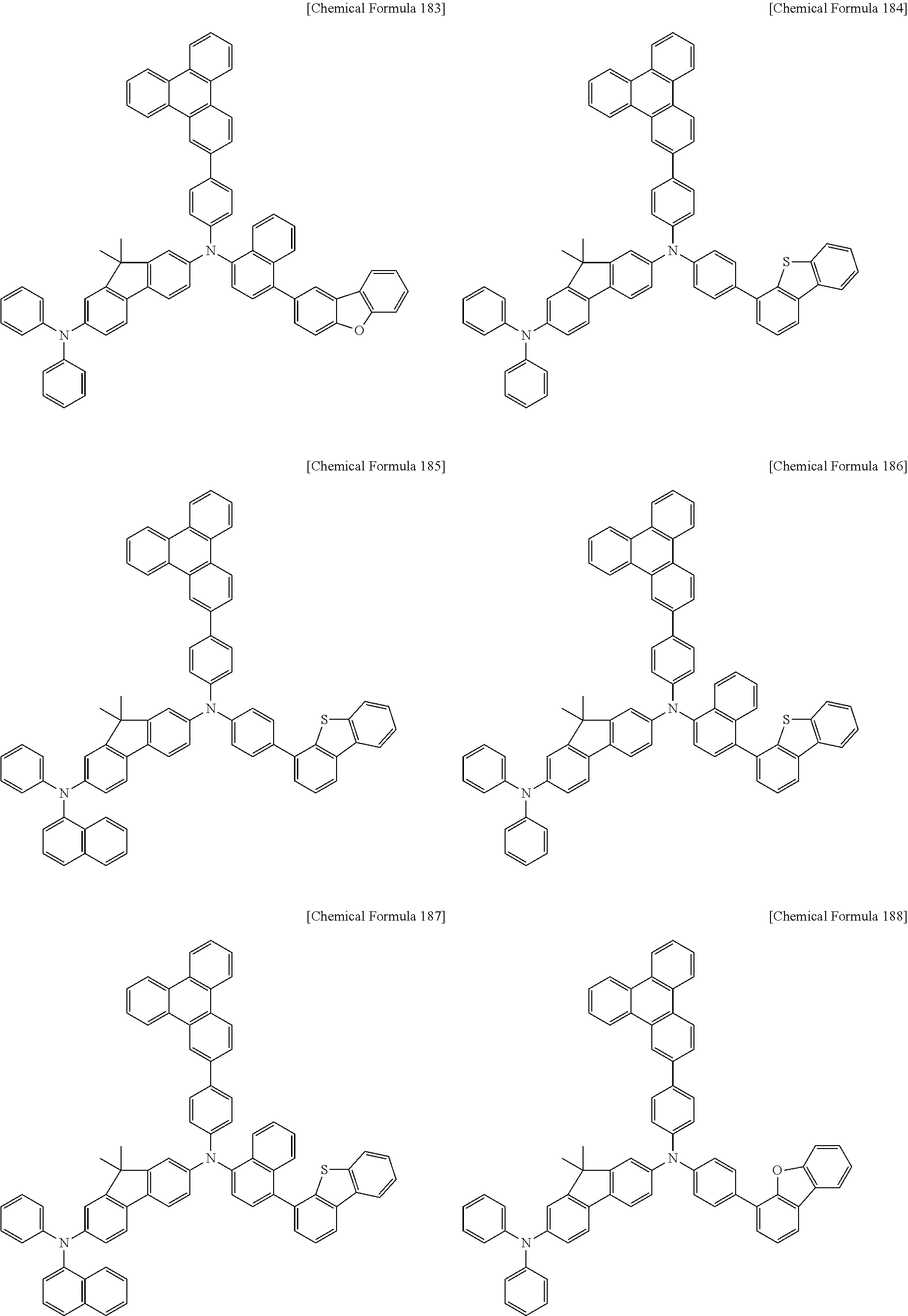

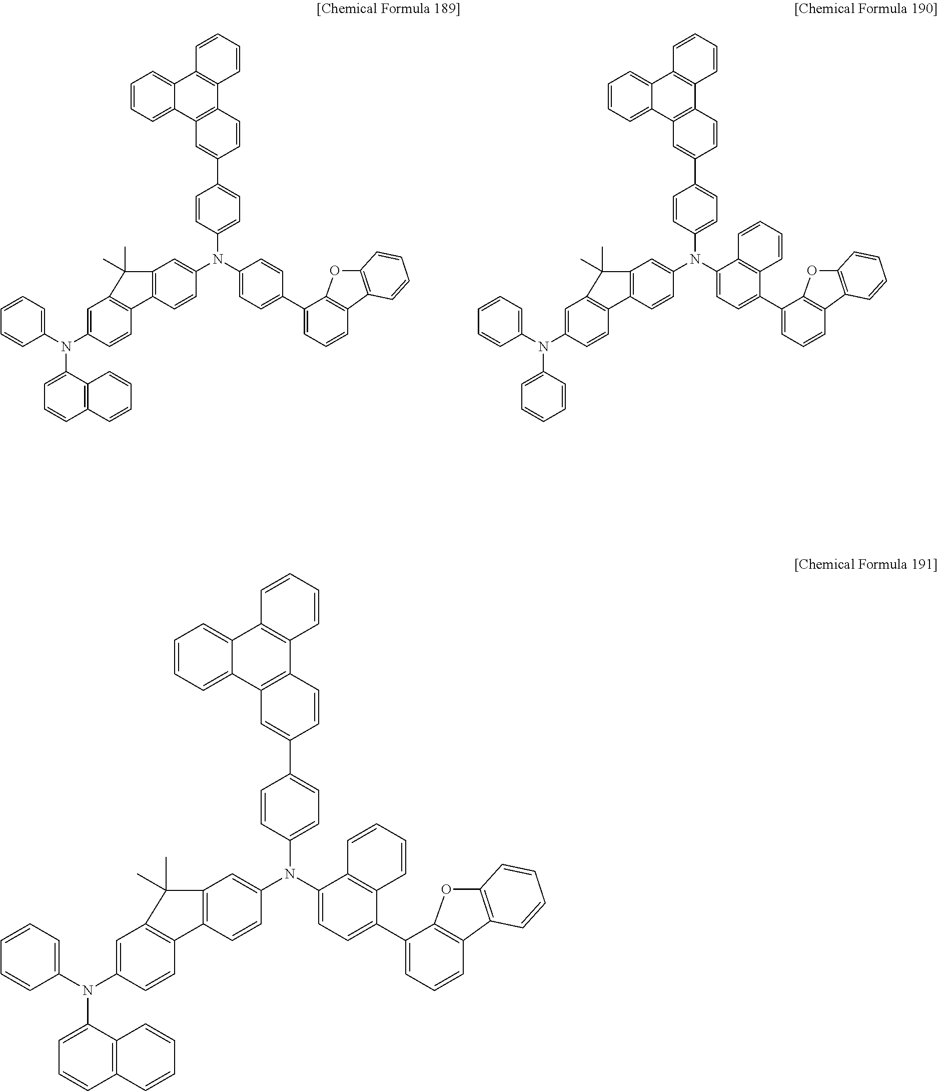 [SMILES: CC1(C)c2cc(N(c3ccc(-c4ccc5c6ccccc6c6ccccc6c5c4)cc3)c3ccc(-c4cccc5c4oc4ccccc45)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4cccc5c4oc4ccccc45)c4ccccc34)cc21 ; CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4cccc5c4oc4ccccc45)c4ccccc34)cc21]

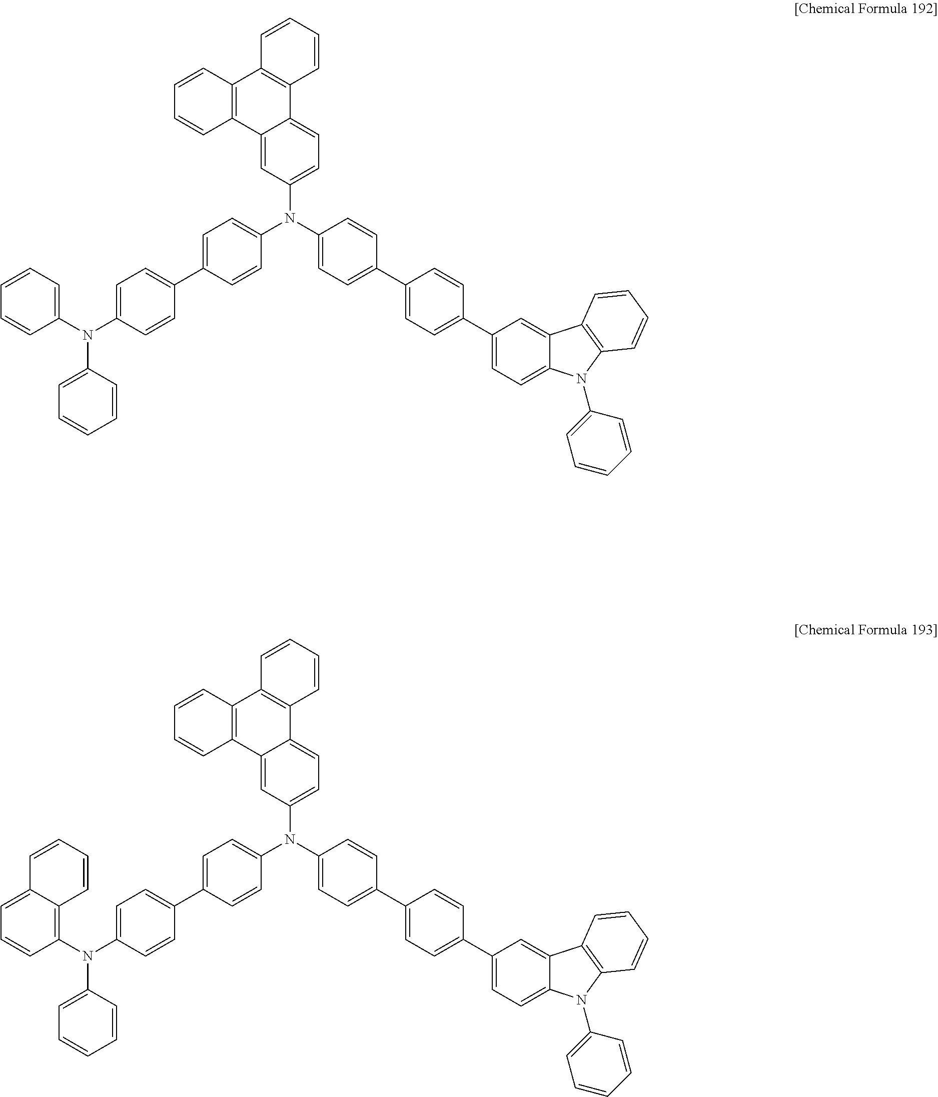 [SMILES: c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)c2cccc3ccccc23)cc1]

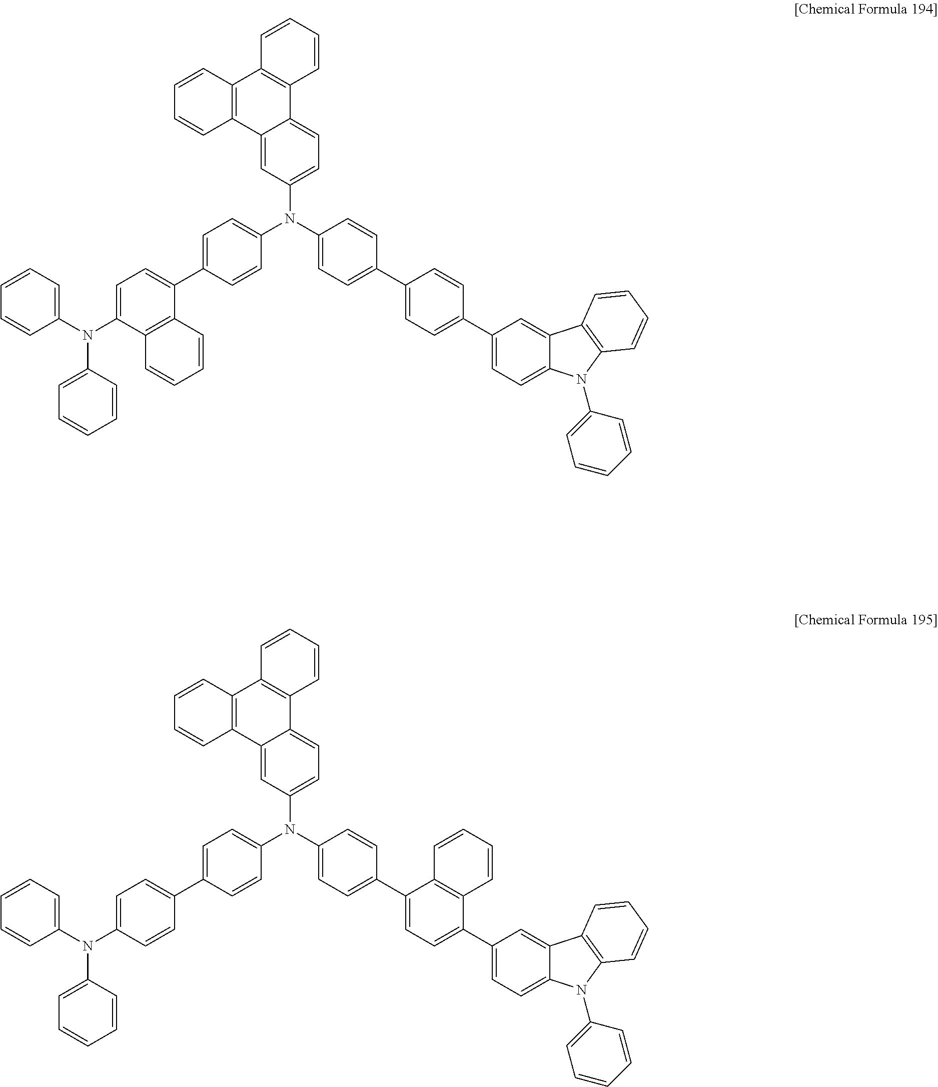 [SMILES: c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)cc2)cc1]

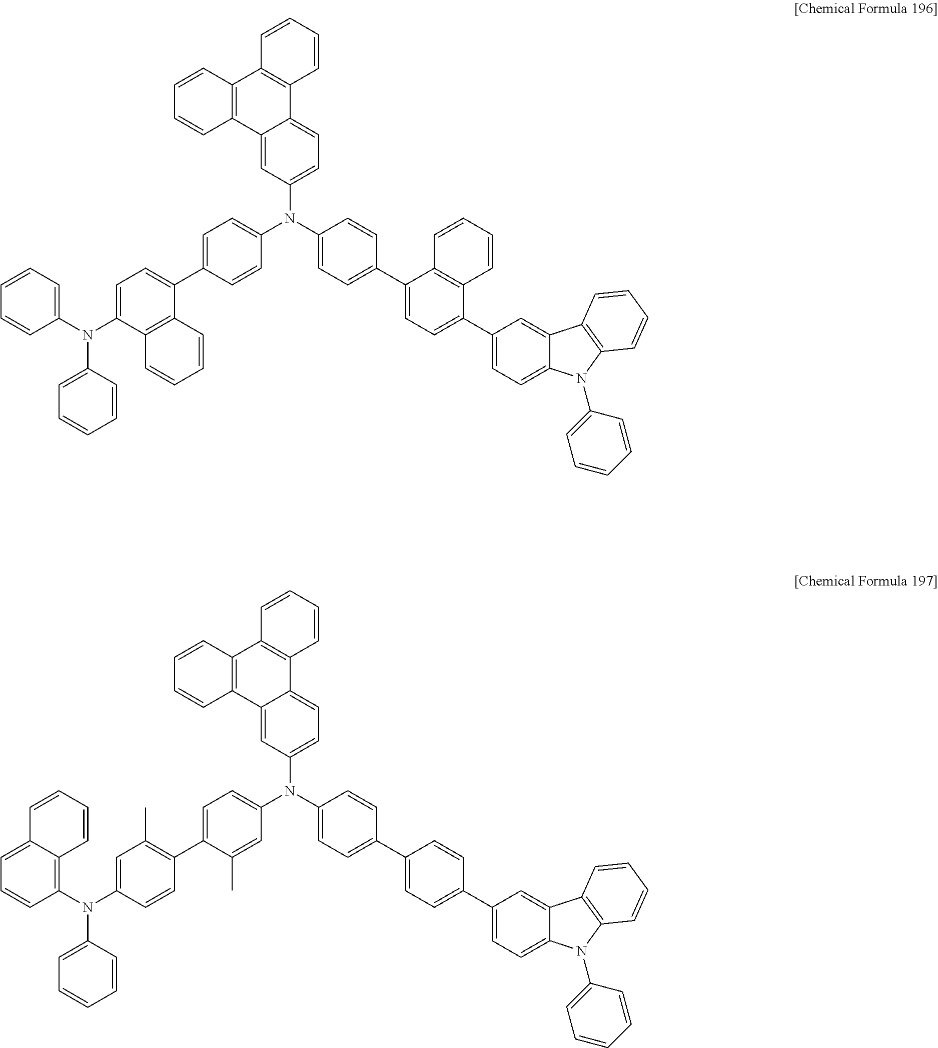 [SMILES: c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)c3ccccc23)cc1 ; Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C]

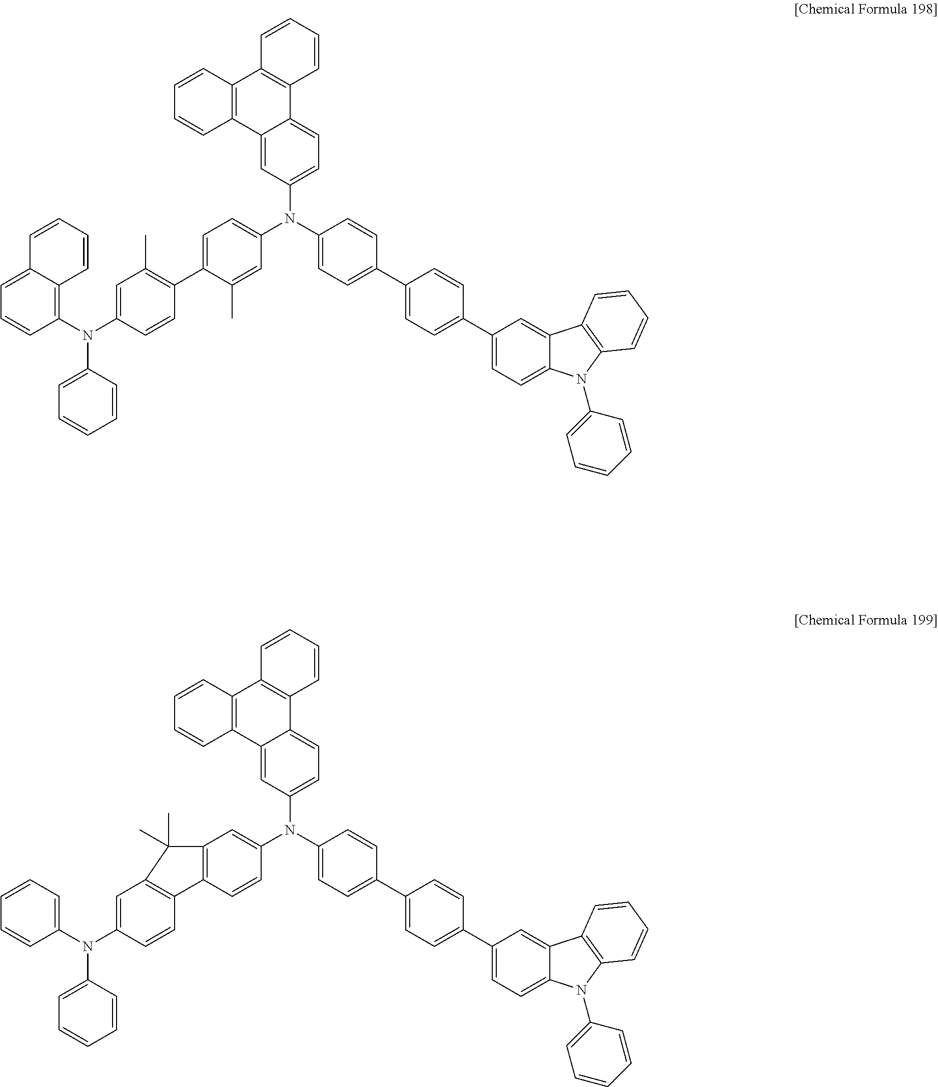 [SMILES: Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C ; CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc4c5ccccc5c5ccccc5c4c3)cc21]

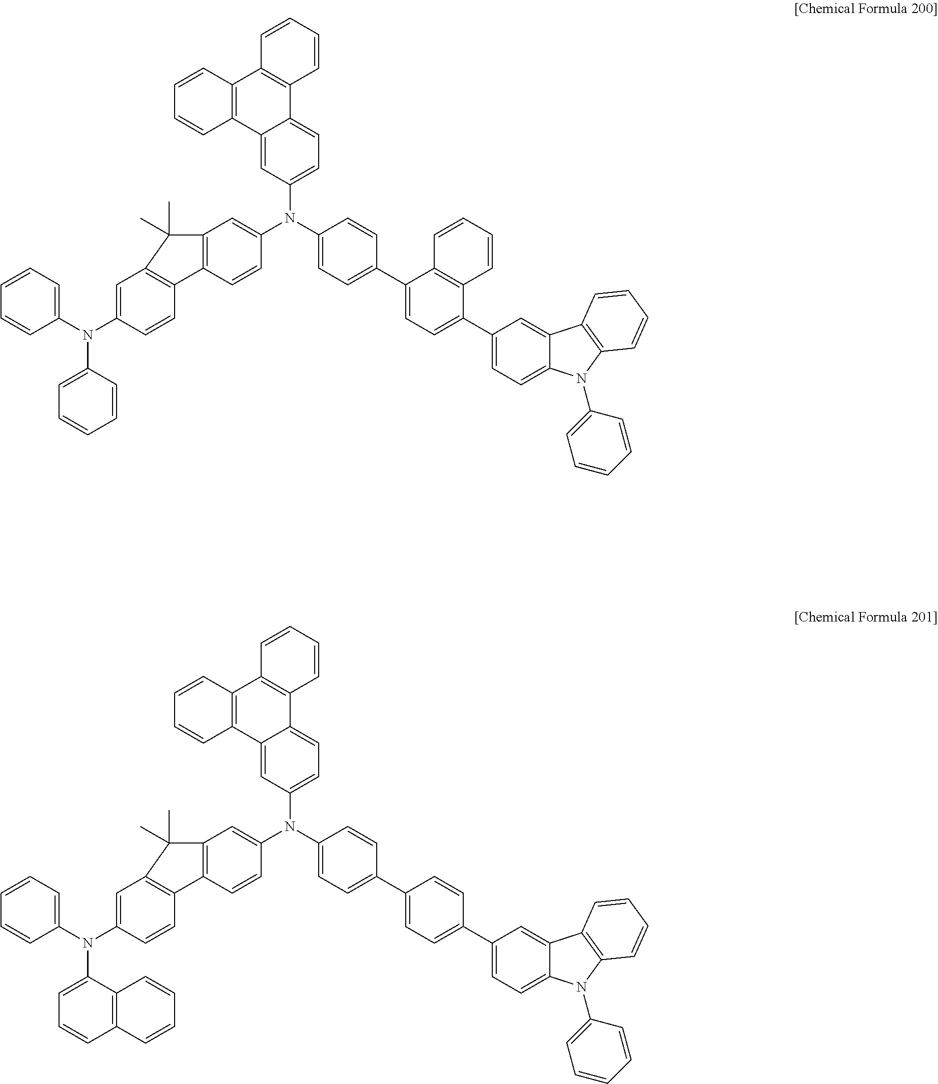 [SMILES: CC1(C)c2cc(N(c3ccccc3)c3ccccc3)ccc2-c2ccc(N(c3ccc(-c4ccc(-c5ccc6c(c5)c5ccccc5n6-c5ccccc5)c5ccccc45)cc3)c3ccc4c5ccccc5c5ccccc5c4c3)cc21 ; CC1(C)c2cc(N(c3ccc(-c4ccc(-c5ccc6c(c5)c5ccccc5n6-c5ccccc5)cc4)cc3)c3ccc4c5ccccc5c5ccccc5c4c3)ccc2-c2ccc(N(c3ccccc3)c3cccc4ccccc34)cc21]

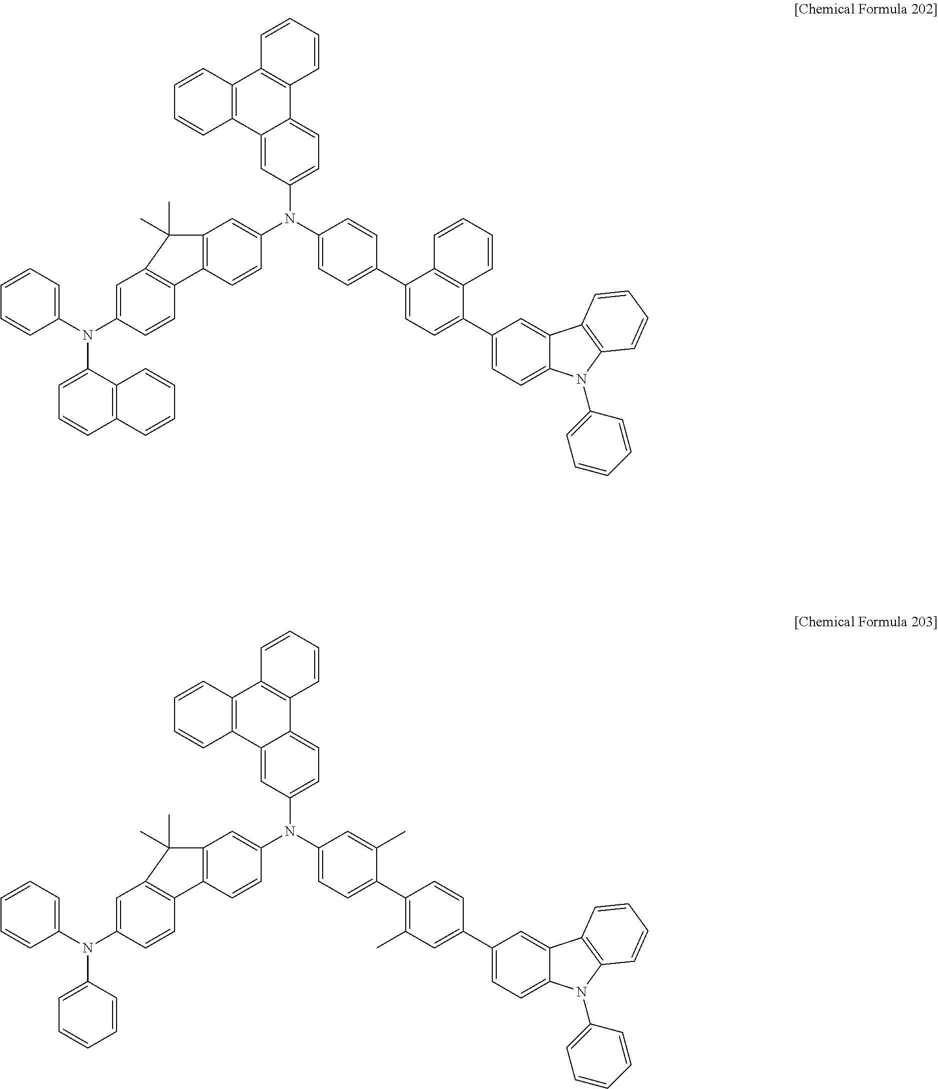 [SMILES: CC1(C)c2cc(N(c3ccc(-c4ccc(-c5ccc6c(c5)c5ccccc5n6-c5ccccc5)c5ccccc45)cc3)c3ccc4c5ccccc5c5ccccc5c4c3)ccc2-c2ccc(N(c3ccccc3)c3cccc4ccccc34)cc21 ; Cc1cc(-c2ccc3c(c2)c2ccccc2n3-c2ccccc2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C]

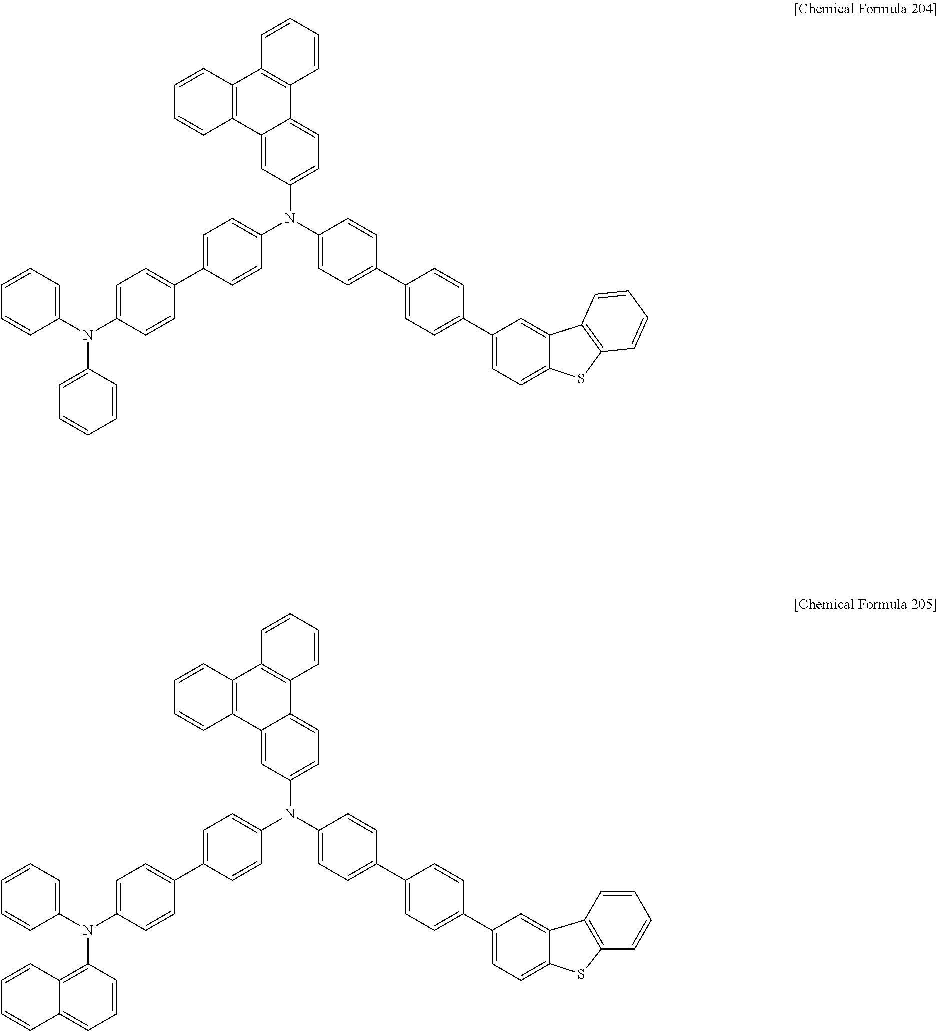 [SMILES: c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7sc8ccccc8c7c6)cc5)cc4)c4ccc5c6ccccc6c6ccccc6c5c4)cc3)cc2)c2cccc3ccccc23)cc1]

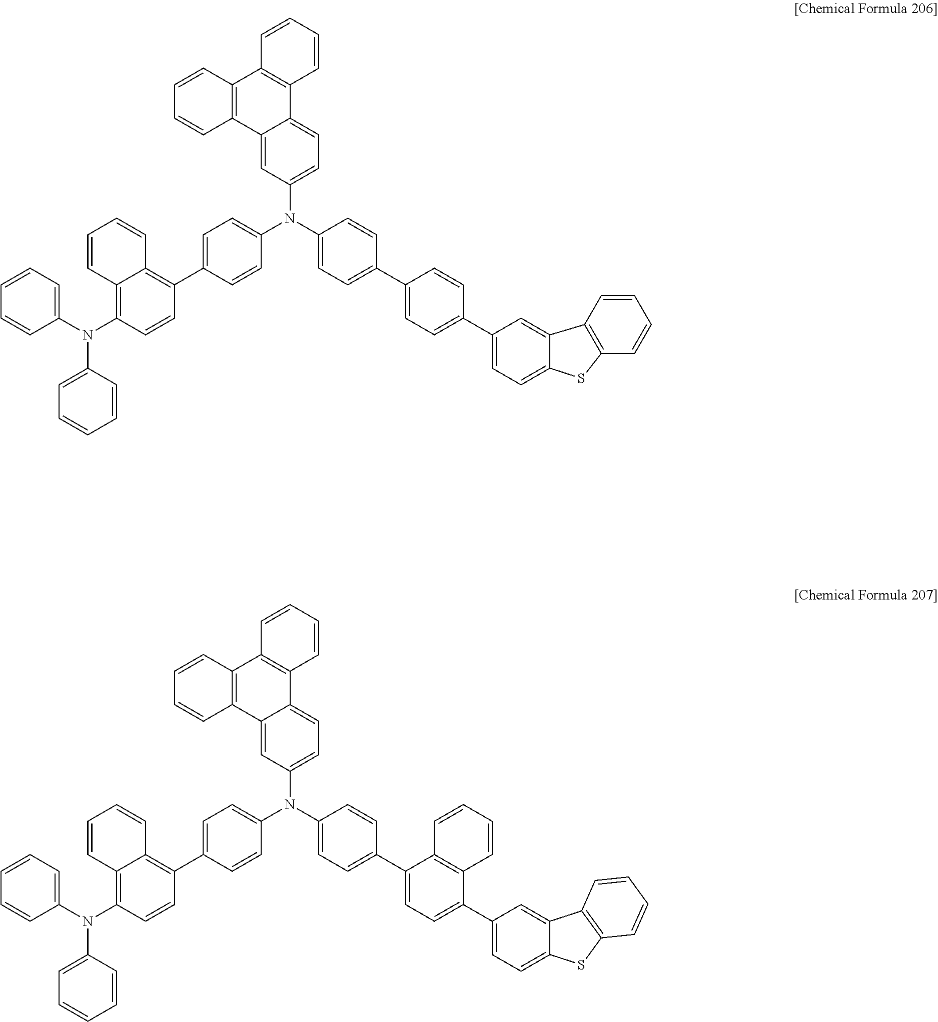 [SMILES: c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7sc8ccccc8c7c6)c6ccccc56)cc4)c4ccc5c6ccccc6c6ccccc6c5c4)cc3)c3ccccc23)cc1]

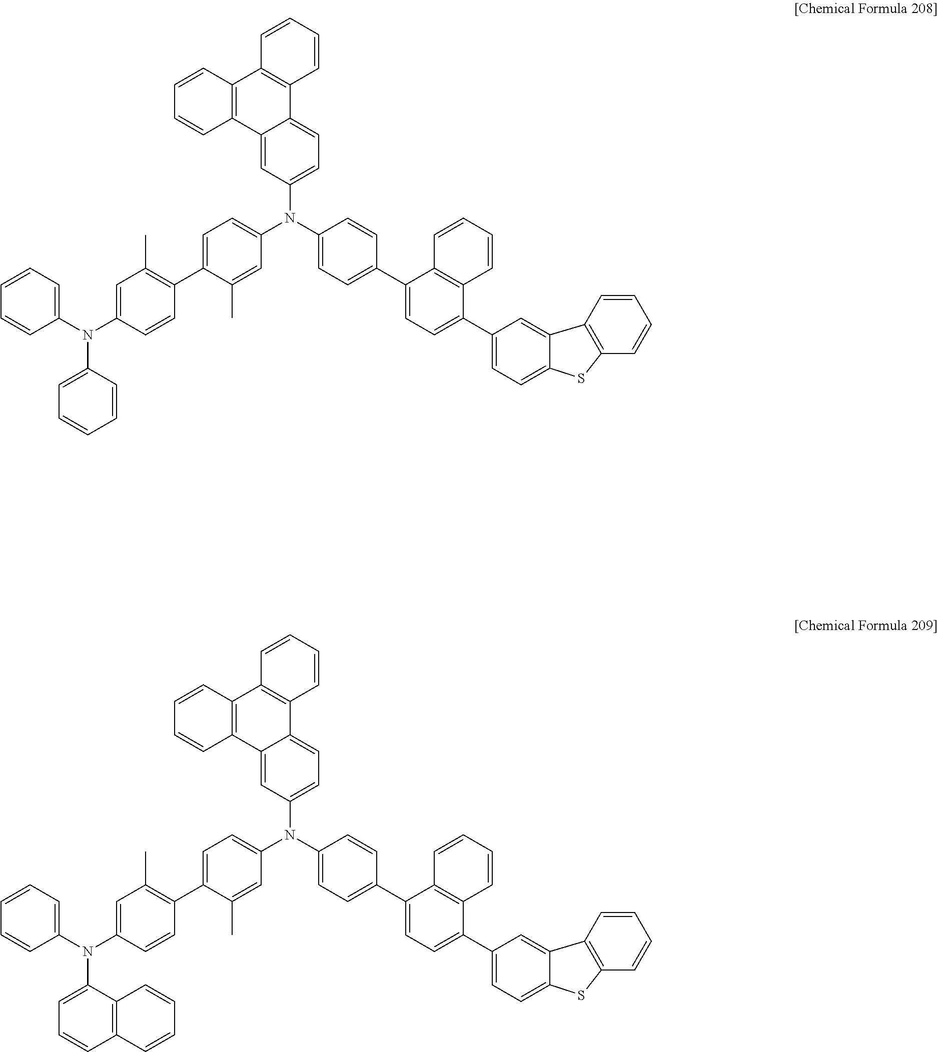 [SMILES: Cc1cc(N(c2ccccc2)c2ccccc2)ccc1-c1ccc(N(c2ccc(-c3ccc(-c4ccc5sc6ccccc6c5c4)c4ccccc34)cc2)c2ccc3c4ccccc4c4ccccc4c3c2)cc1C ; Cc1cc(N(c2ccc(-c3ccc(-c4ccc5sc6ccccc6c5c4)c4ccccc34)cc2)c2ccc3c4ccccc4c4ccccc4c3c2)ccc1-c1ccc(N(c2ccccc2)c2cccc3ccccc23)cc1C]

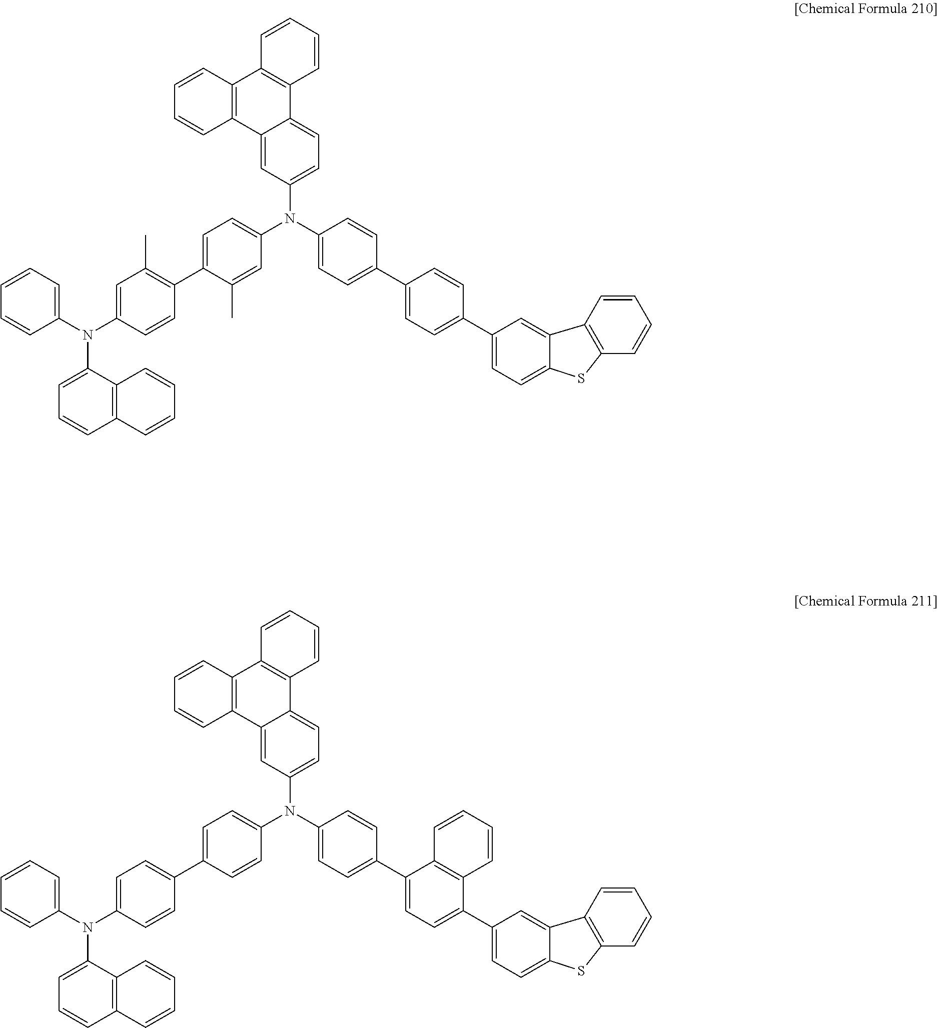 [SMILES: Cc1cc(N(c2ccc(-c3ccc(-c4ccc5sc6ccccc6c5c4)cc3)cc2)c2ccc3c4ccccc4c4ccccc4c3c2)ccc1-c1ccc(N(c2ccccc2)c2cccc3ccccc23)cc1C ; c1ccc(N(c2ccc(-c3ccc(N(c4ccc(-c5ccc(-c6ccc7sc8ccccc8c7c6)c6ccccc56)cc4)c4ccc5c6ccccc6c6ccccc6c5c4)cc3)cc2)c2cccc3ccccc23)cc1]

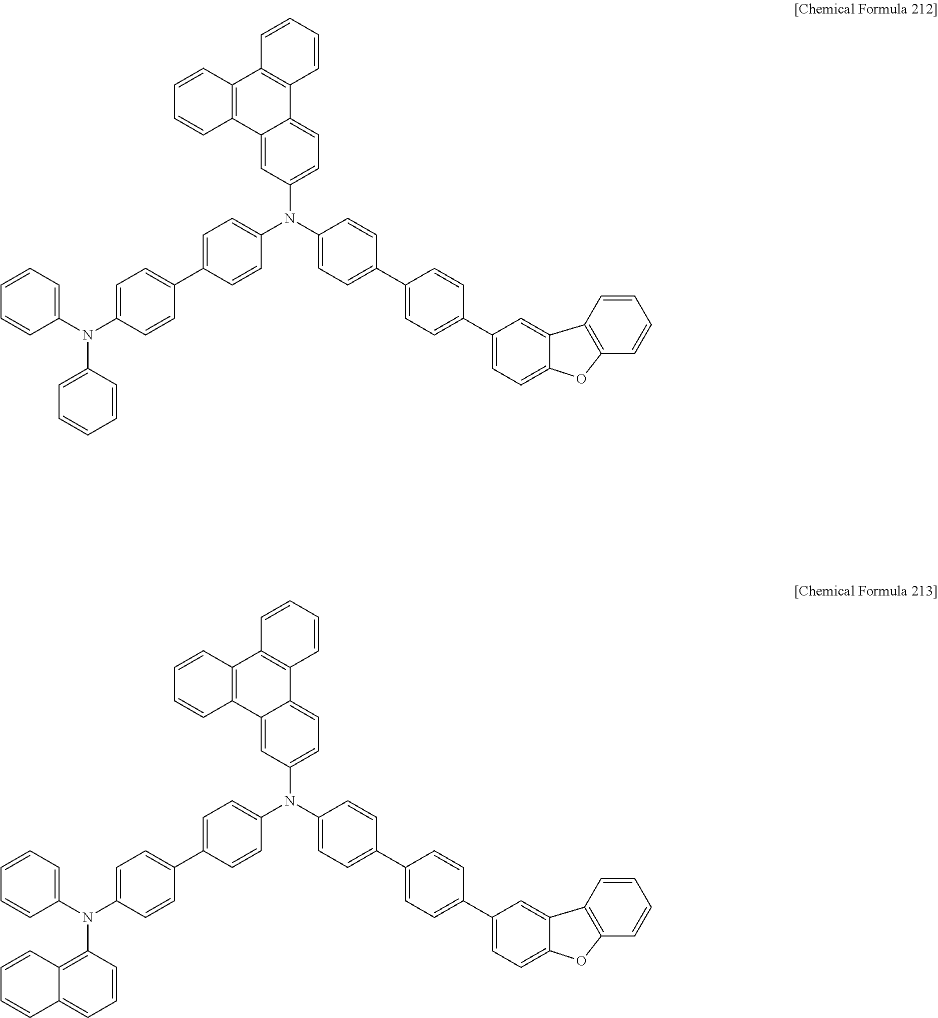 [SMILES: c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7oc8ccccc8c7c6)cc5)cc4)c4ccc5c6ccccc6c6ccccc6c5c4)cc3)cc2)c2cccc3ccccc23)cc1]

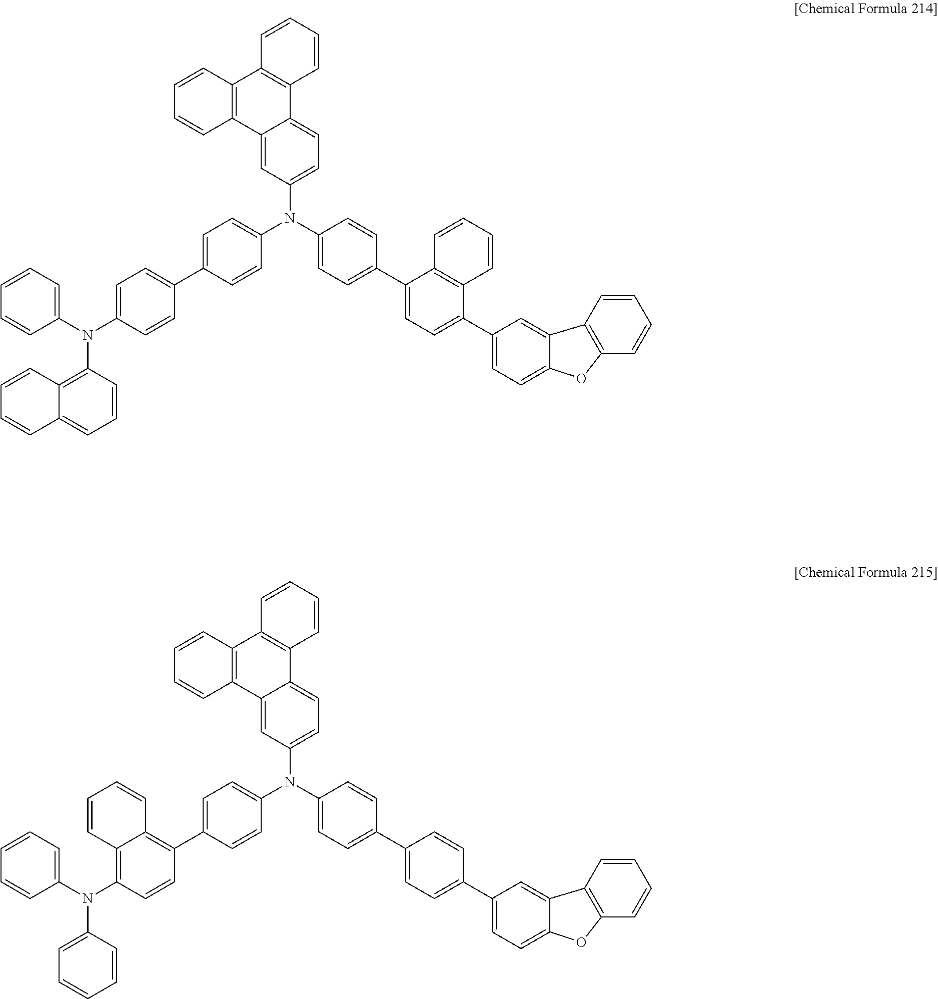 [SMILES: c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)c3ccccc23)cc1]

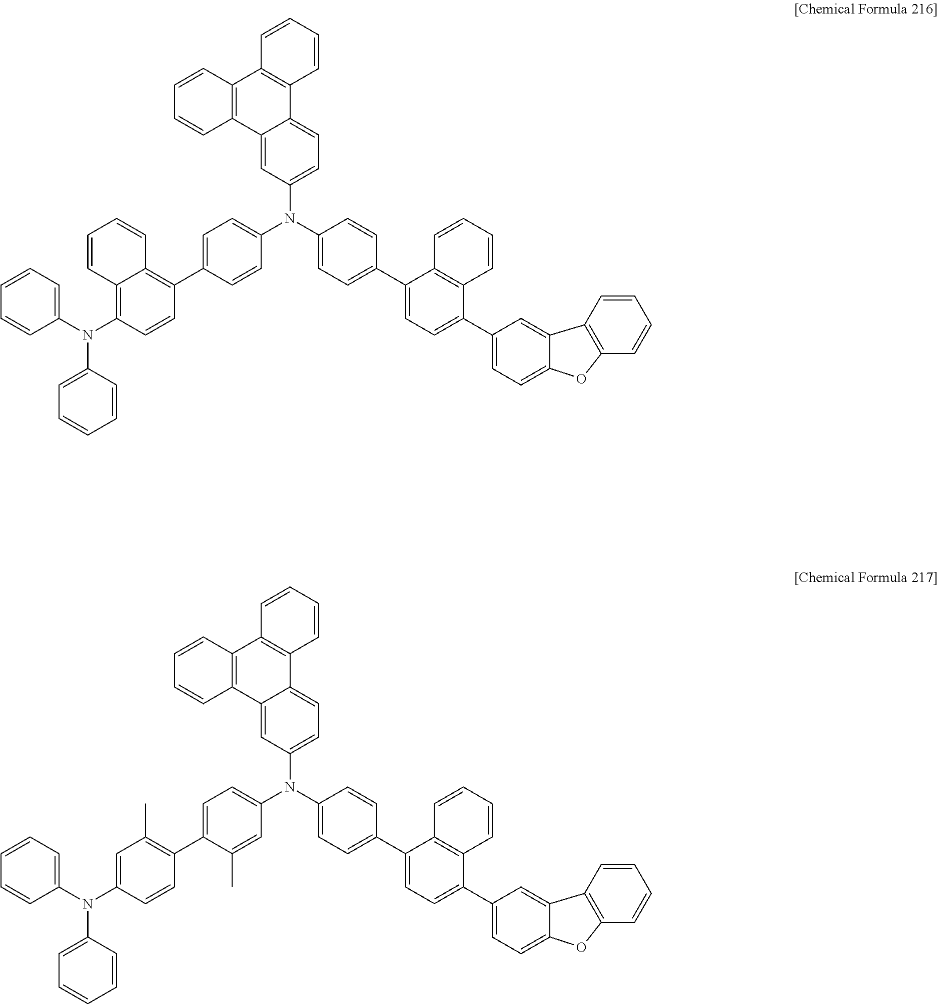 [SMILES: c1ccc(N(c2ccccc2)c2ccc(-c3ccc(N(c4ccc(-c5ccc(-c6ccc7oc8ccccc8c7c6)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4ccc5oc6ccccc6c5c4)c4ccccc34)cc2)c2ccc3c4ccccc4c4ccccc4c3c2)cc1C]

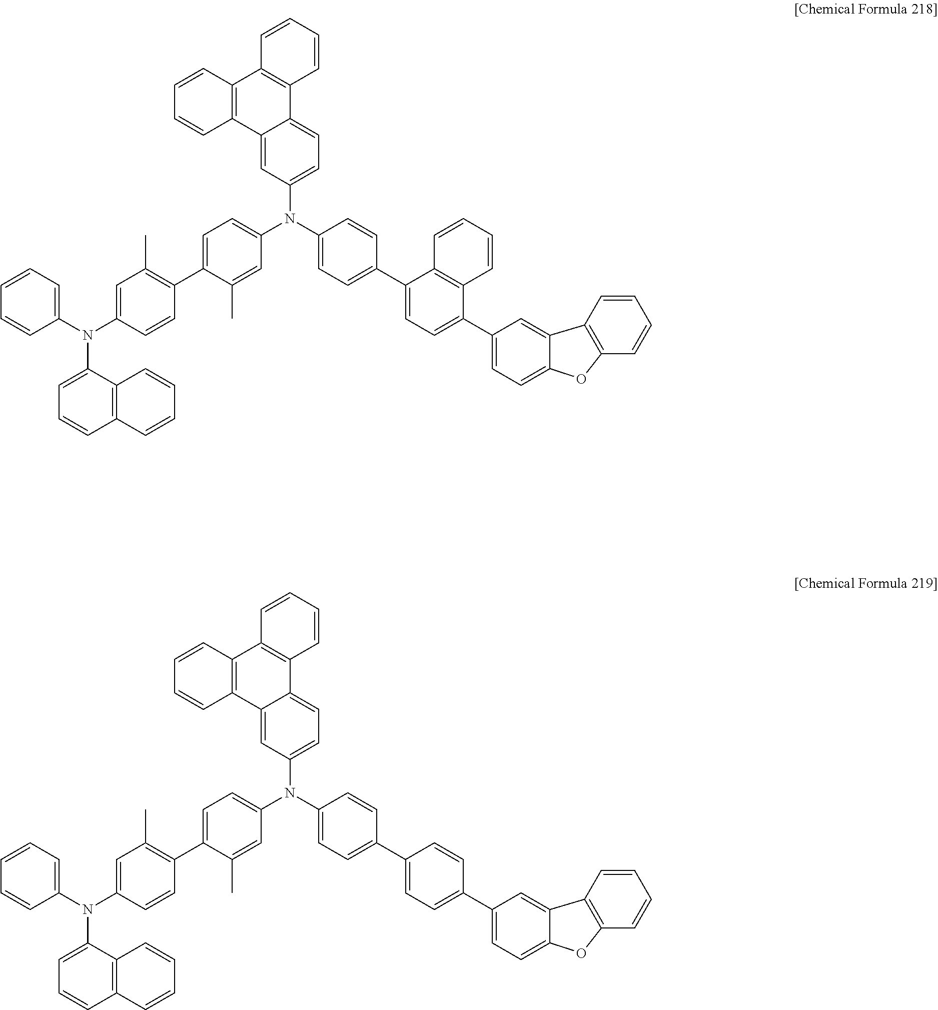 [SMILES: Cc1cc(N(c2ccc(-c3ccc(-c4ccc5oc6ccccc6c5c4)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5oc6ccccc6c5c4)cc3)cc2)c2ccc3c4ccccc4c4ccccc4c3c2)ccc1-c1ccc(N(c2ccccc2)c2cccc3ccccc23)cc1C]

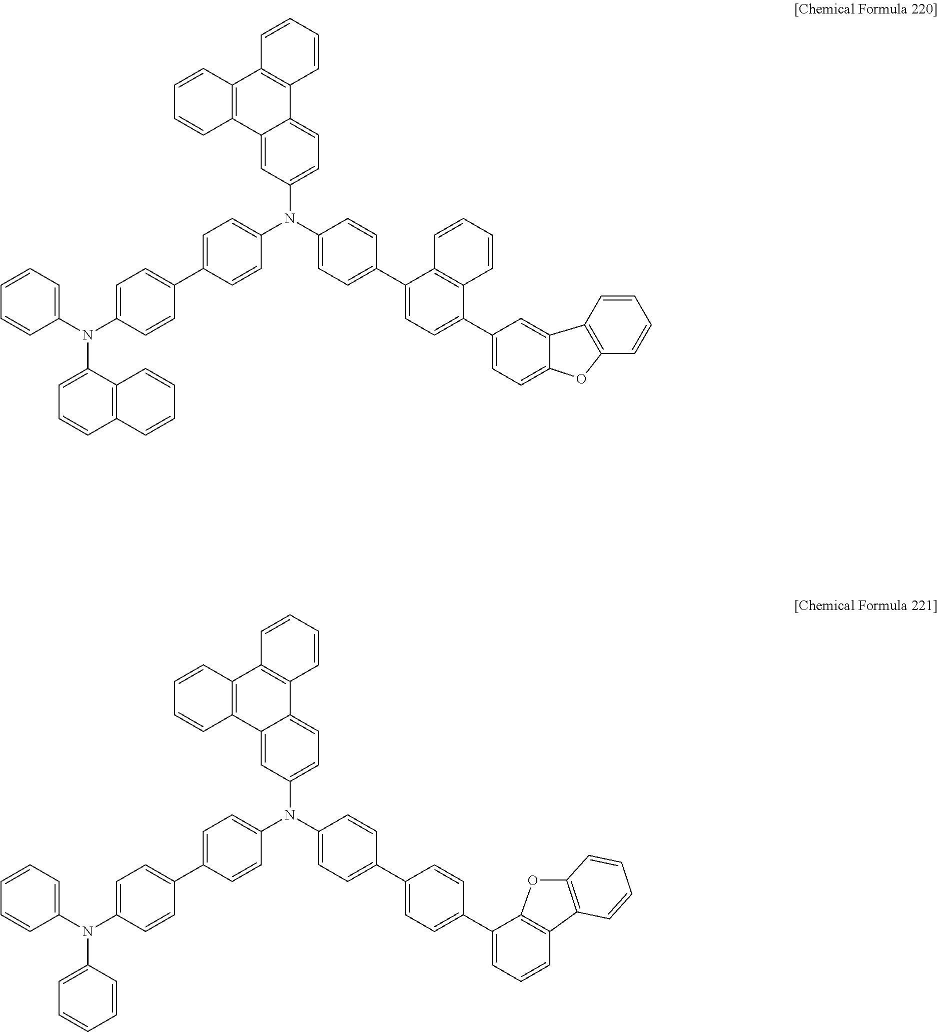 [SMILES: c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)cc2)cc1]

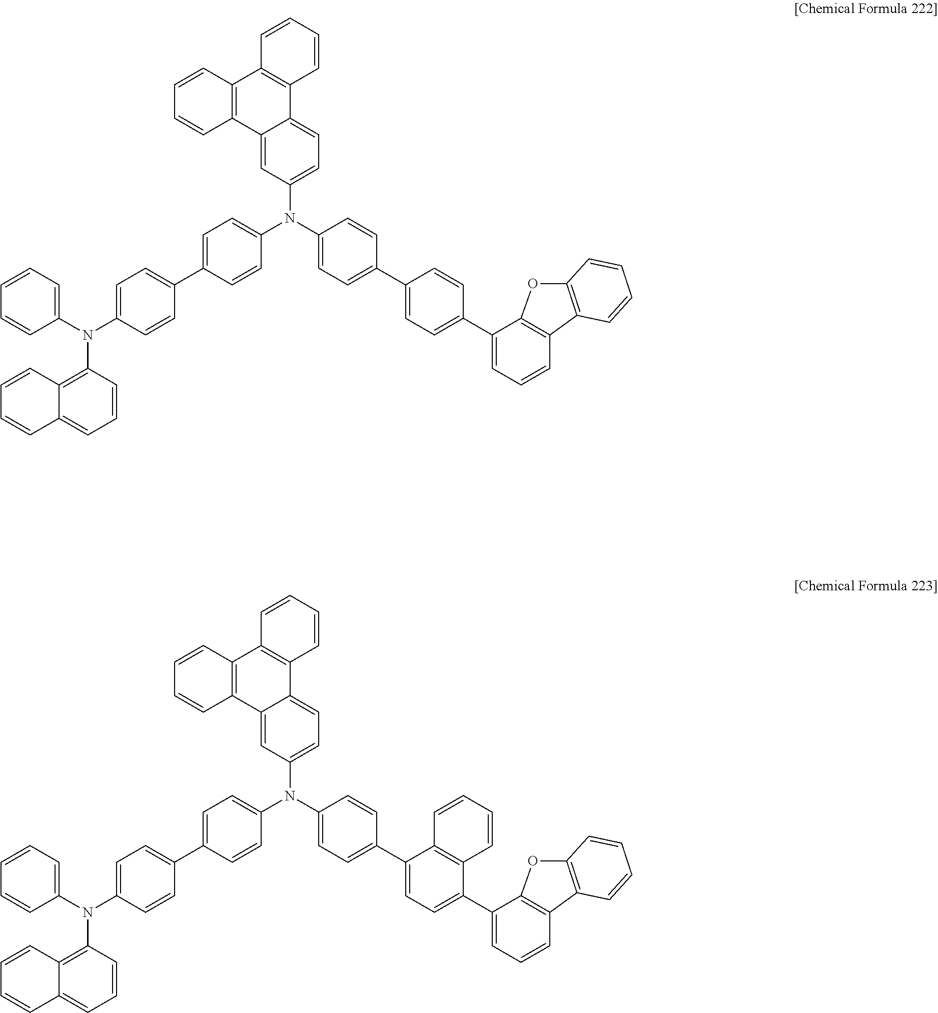 [SMILES: c1ccc(N(c2ccc(-c3ccc(N(c4ccc(-c5ccc(-c6cccc7c6oc6ccccc67)cc5)cc4)c4ccc5c6ccccc6c6ccccc6c5c4)cc3)cc2)c2cccc3ccccc23)cc1 ; c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1]

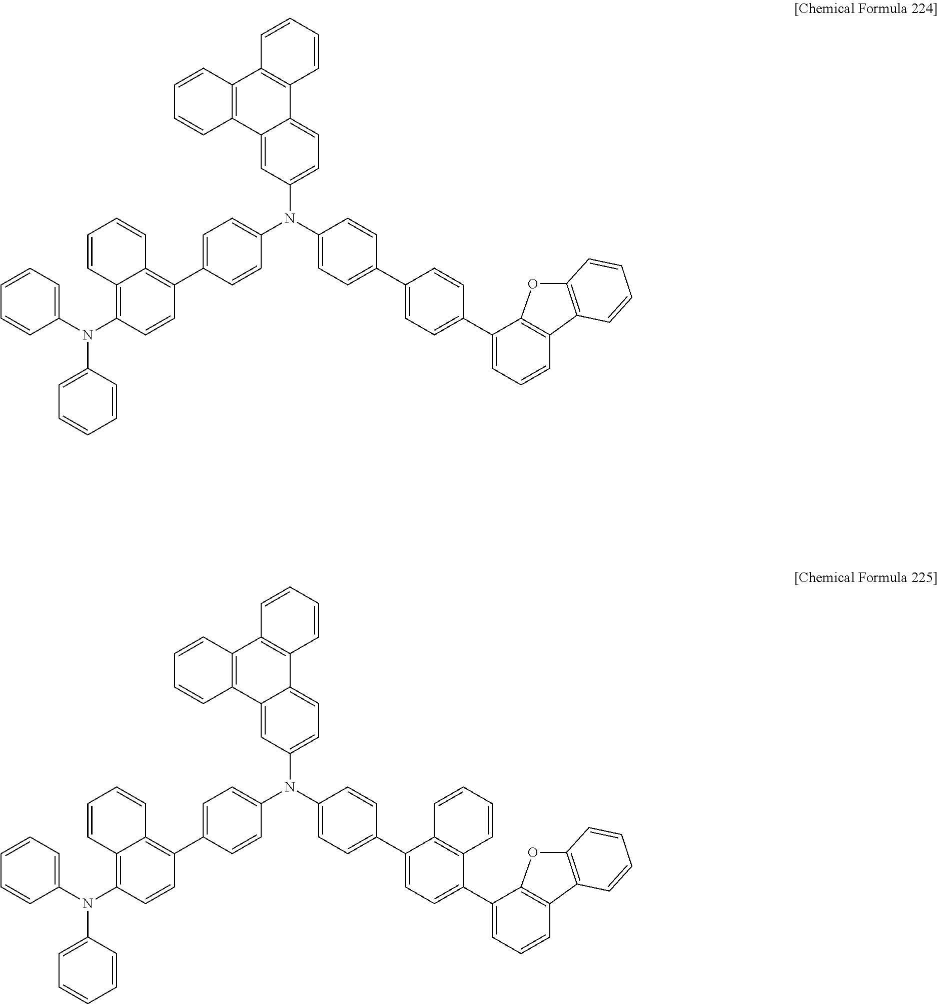 [SMILES: c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6cccc7c6oc6ccccc67)c6ccccc56)cc4)c4ccc5c6ccccc6c6ccccc6c5c4)cc3)c3ccccc23)cc1]

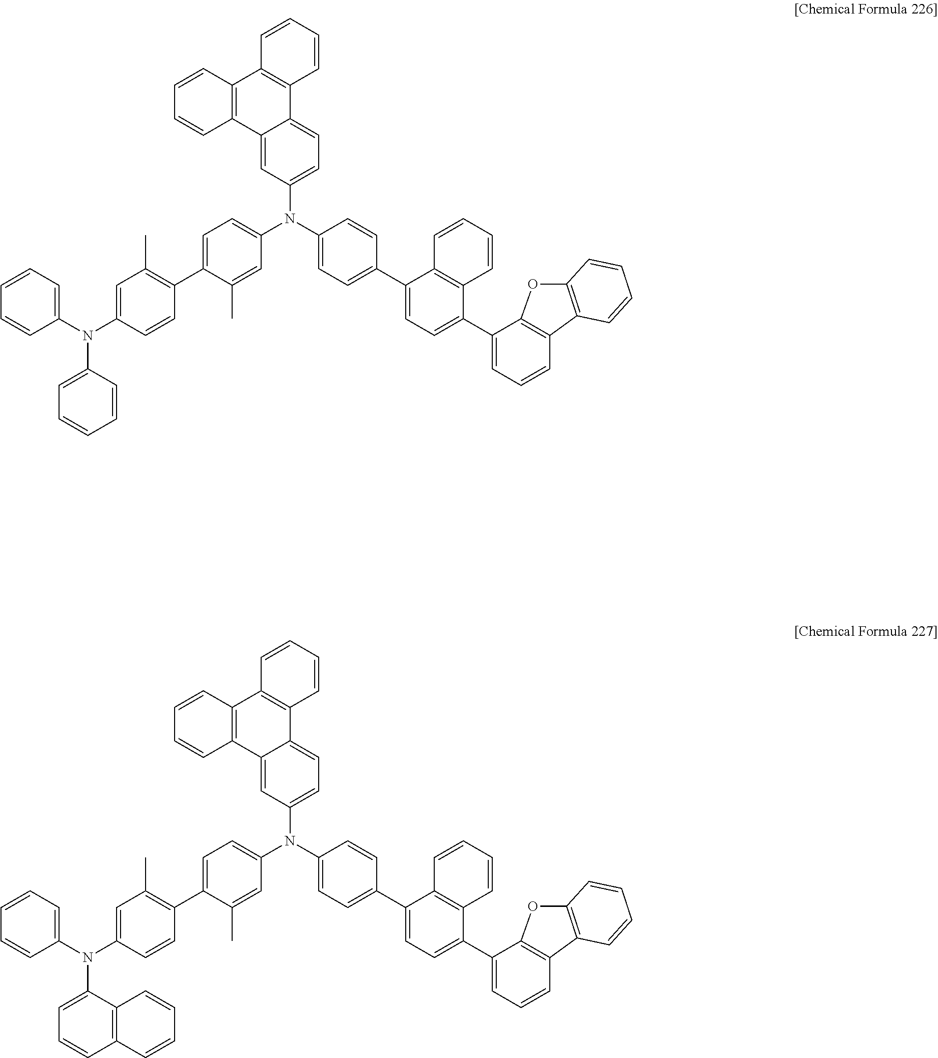 [SMILES: Cc1cc(N(c2ccccc2)c2ccccc2)ccc1-c1ccc(N(c2ccc(-c3ccc(-c4cccc5c4oc4ccccc45)c4ccccc34)cc2)c2ccc3c4ccccc4c4ccccc4c3c2)cc1C ; Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C]

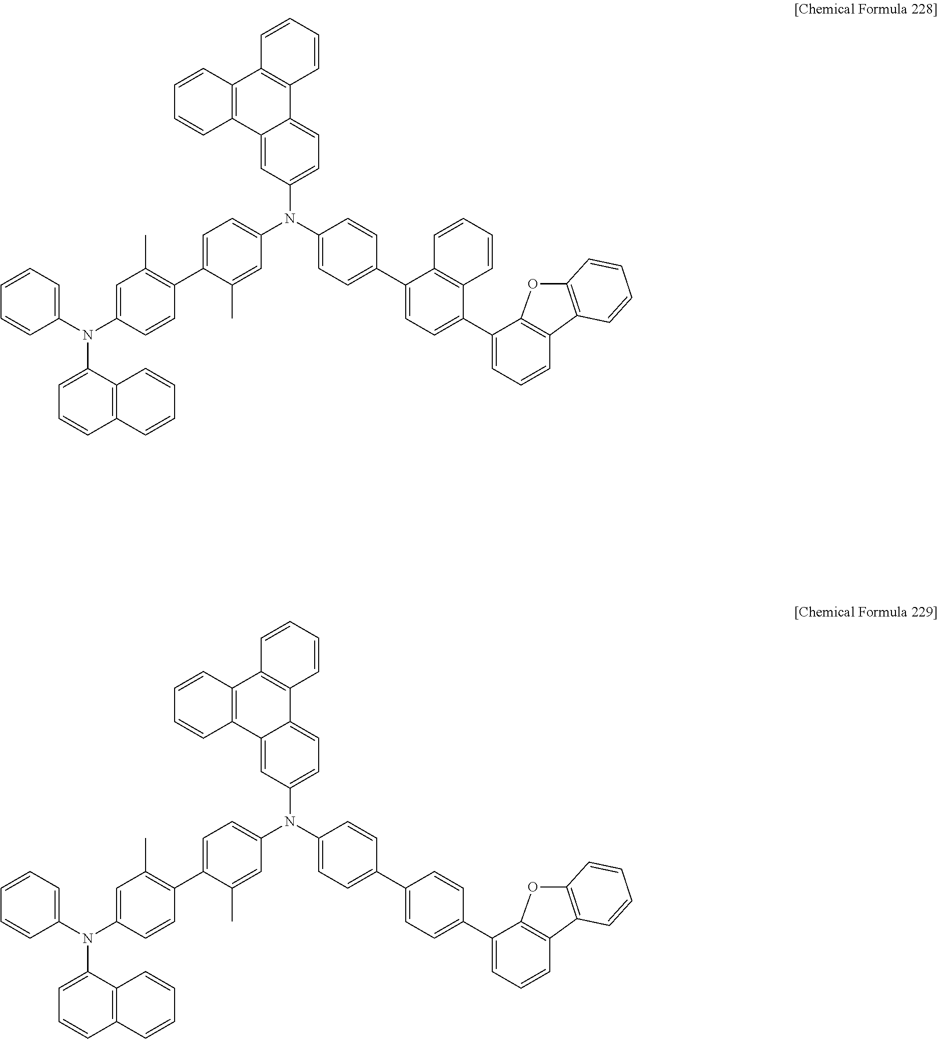 [SMILES: Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4cccc5c4oc4ccccc45)cc3)cc2)c2ccc3c4ccccc4c4ccccc4c3c2)ccc1-c1ccc(N(c2ccccc2)c2cccc3ccccc23)cc1C]

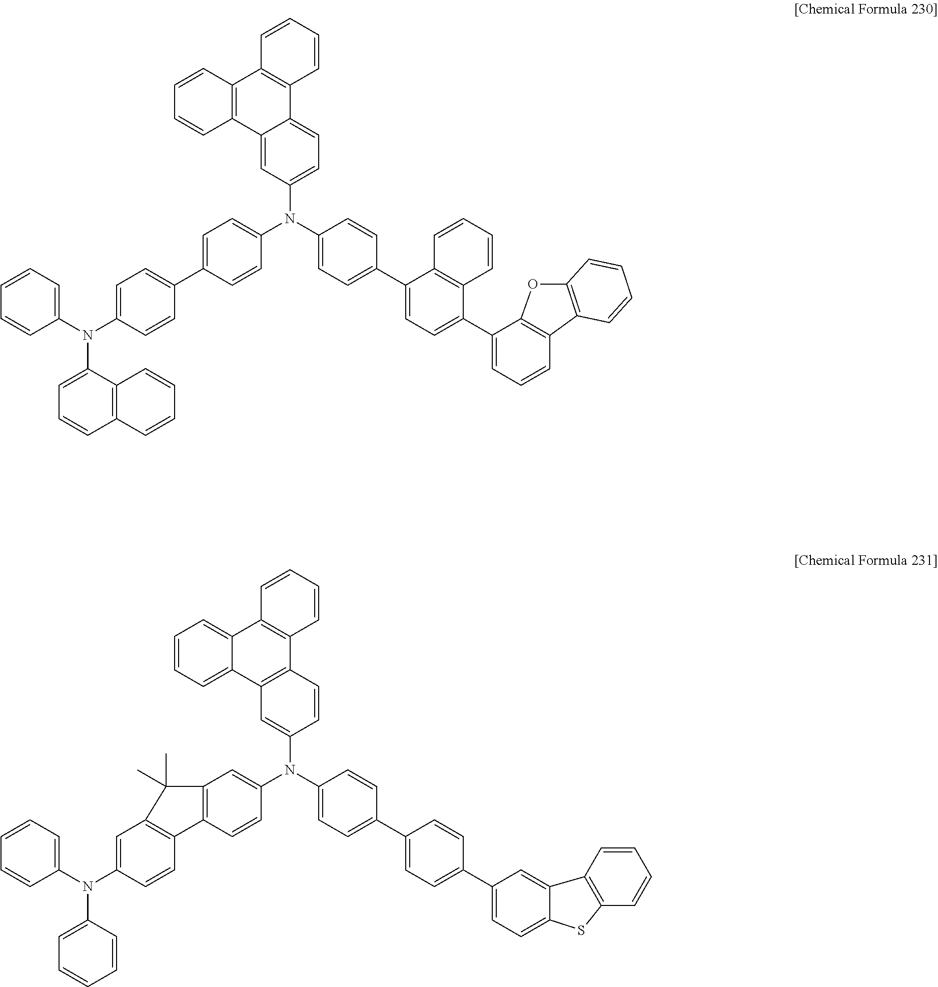 [SMILES: c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1 ; CC1(C)c2cc(N(c3ccccc3)c3ccccc3)ccc2-c2ccc(N(c3ccc(-c4ccc(-c5ccc6sc7ccccc7c6c5)cc4)cc3)c3ccc4c5ccccc5c5ccccc5c4c3)cc21]

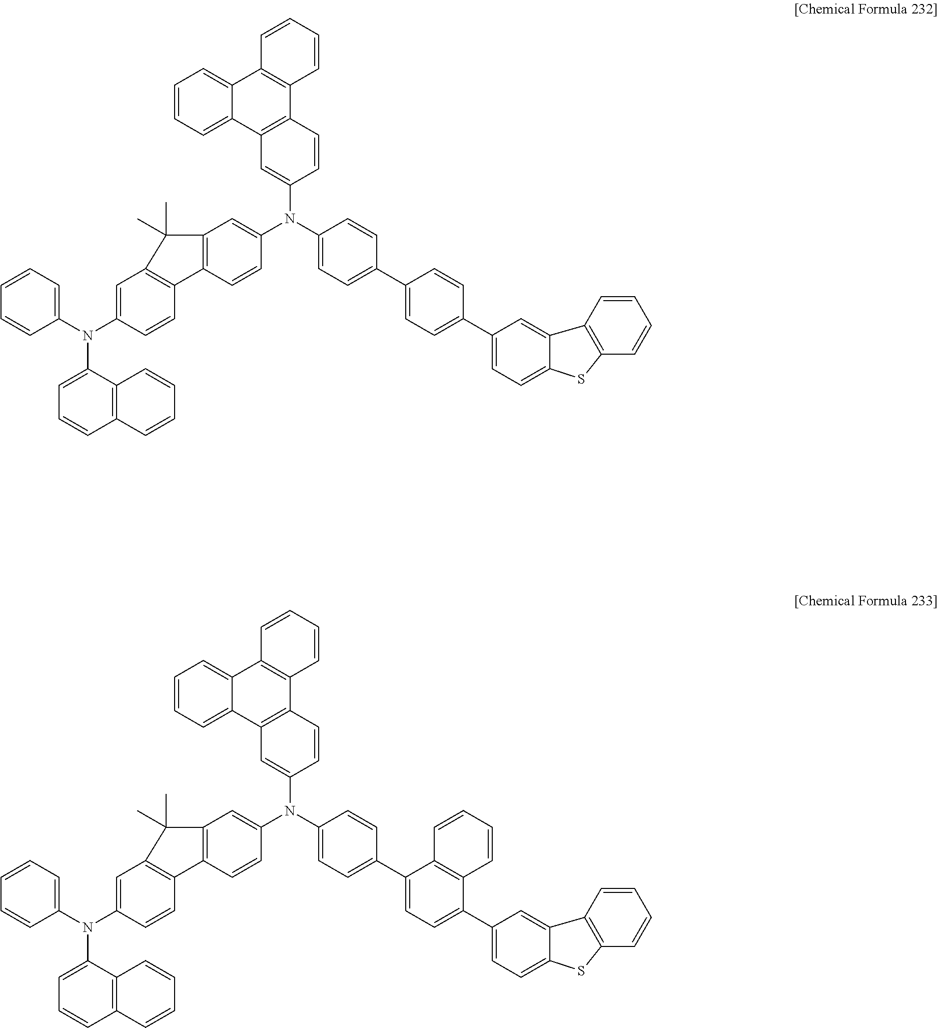 [SMILES: CC1(C)c2cc(N(c3ccc(-c4ccc(-c5ccc6sc7ccccc7c6c5)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccc(-c4ccc(-c5ccc6sc7ccccc7c6c5)c5ccccc45)cc3)c3ccc4c5ccccc5c5ccccc5c4c3)ccc2-c2ccc(N(c3ccccc3)c3cccc4ccccc34)cc21]

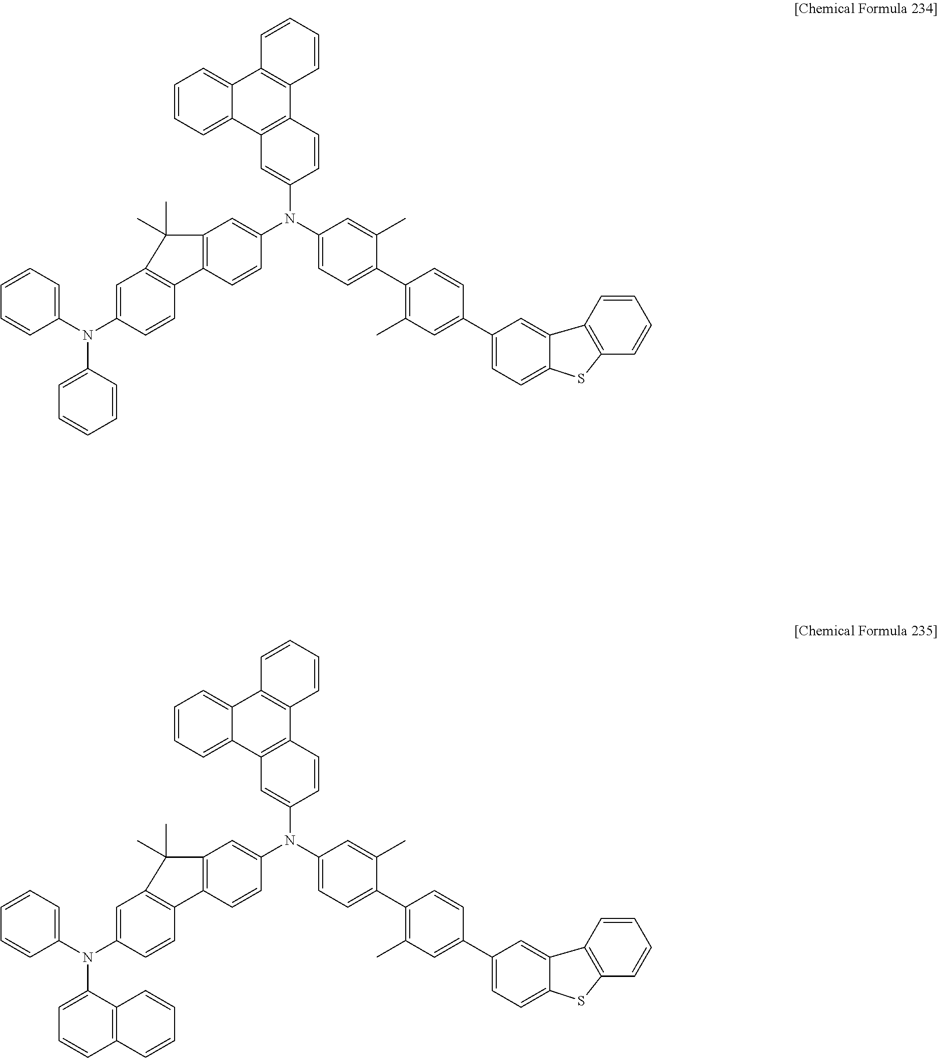 [SMILES: Cc1cc(-c2ccc3sc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2ccc3sc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

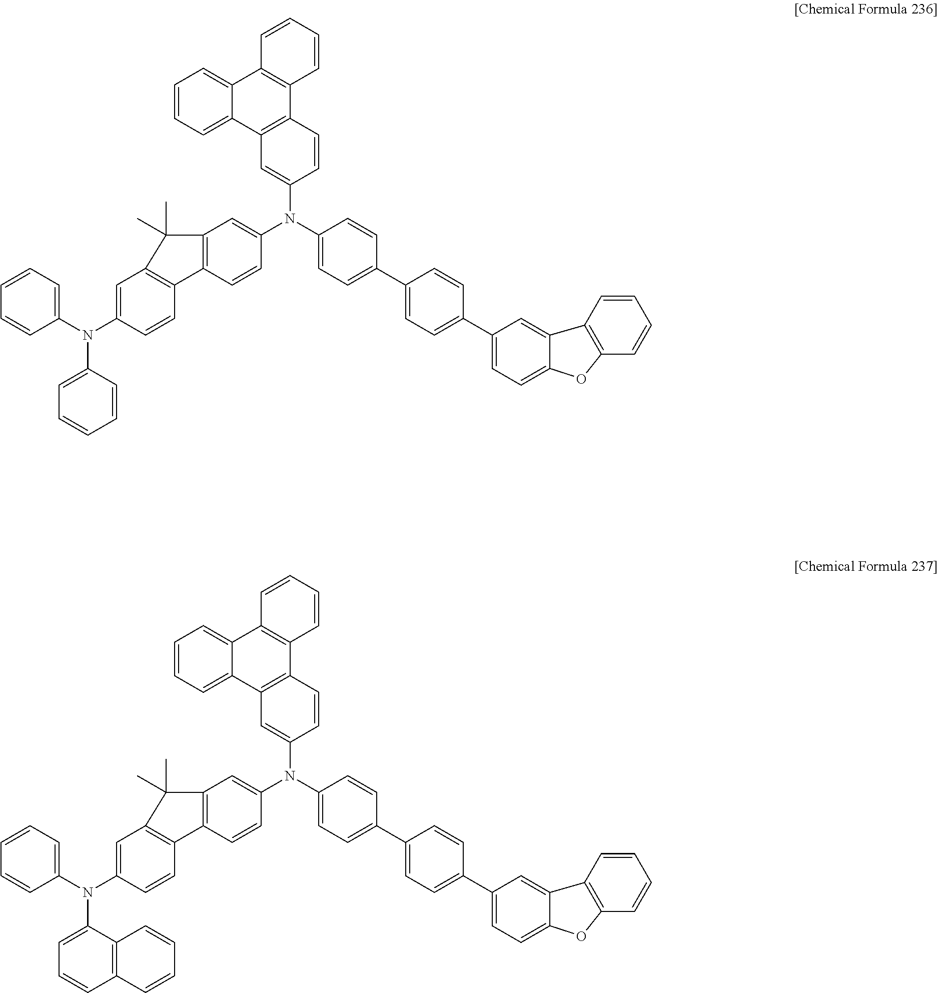 [SMILES: CC1(C)c2cc(N(c3ccccc3)c3ccccc3)ccc2-c2ccc(N(c3ccc(-c4ccc(-c5ccc6oc7ccccc7c6c5)cc4)cc3)c3ccc4c5ccccc5c5ccccc5c4c3)cc21 ; CC1(C)c2cc(N(c3ccc(-c4ccc(-c5ccc6oc7ccccc7c6c5)cc4)cc3)c3ccc4c5ccccc5c5ccccc5c4c3)ccc2-c2ccc(N(c3ccccc3)c3cccc4ccccc34)cc21]

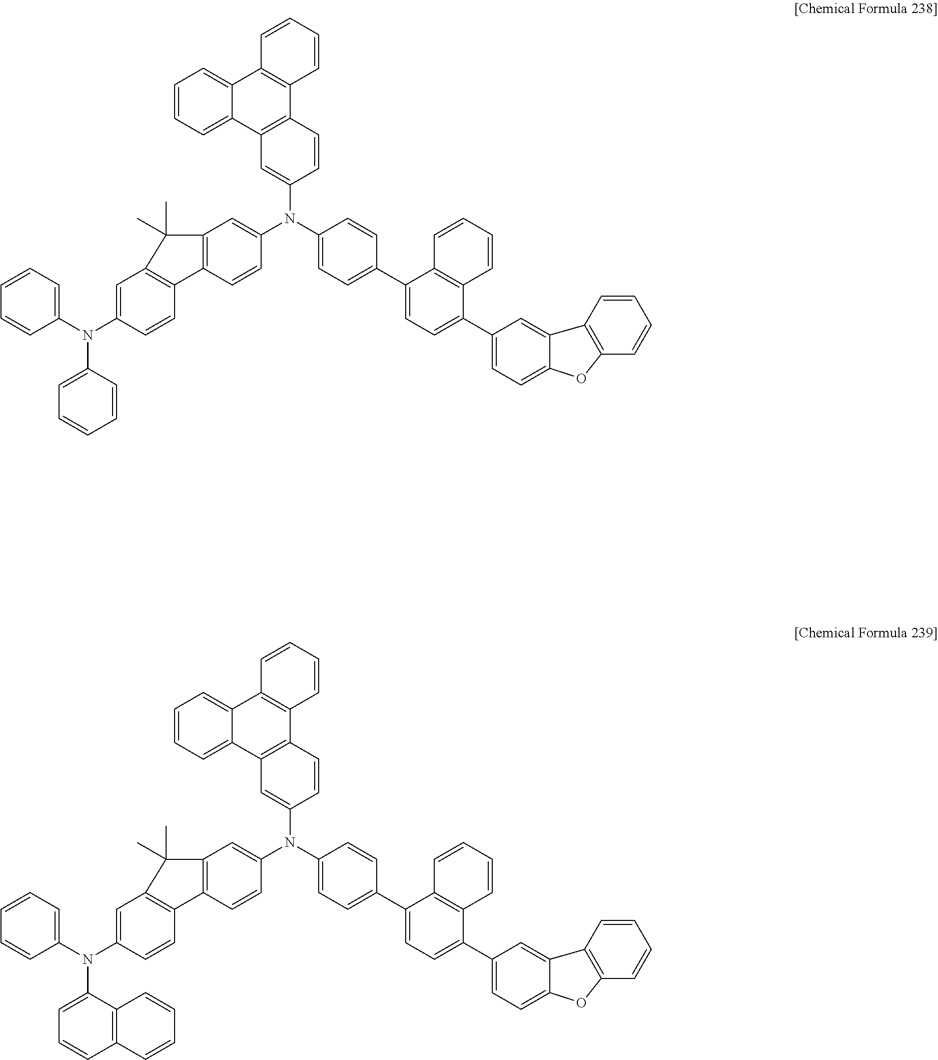 [SMILES: CC1(C)c2cc(N(c3ccccc3)c3ccccc3)ccc2-c2ccc(N(c3ccc(-c4ccc(-c5ccc6oc7ccccc7c6c5)c5ccccc45)cc3)c3ccc4c5ccccc5c5ccccc5c4c3)cc21 ; CC1(C)c2cc(N(c3ccc(-c4ccc(-c5ccc6oc7ccccc7c6c5)c5ccccc45)cc3)c3ccc4c5ccccc5c5ccccc5c4c3)ccc2-c2ccc(N(c3ccccc3)c3cccc4ccccc34)cc21]

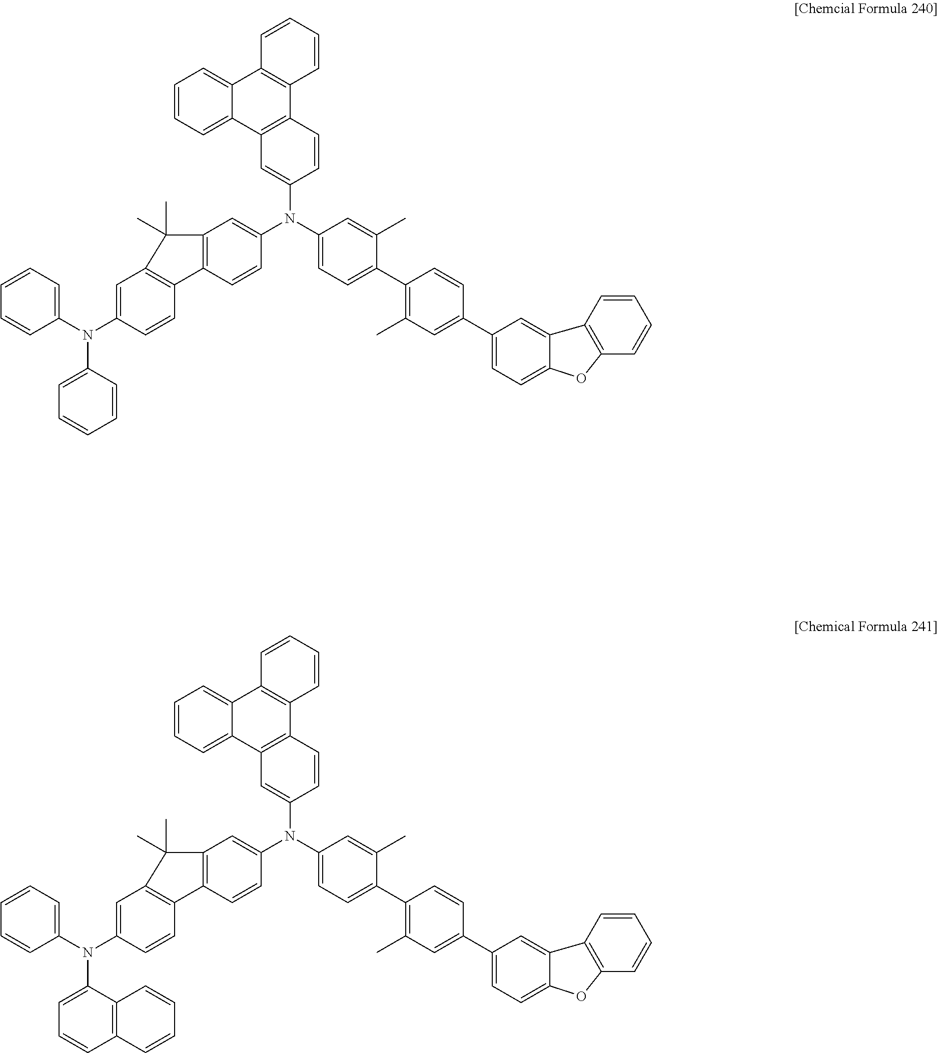 [SMILES: Cc1cc(-c2ccc3oc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2ccc3oc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

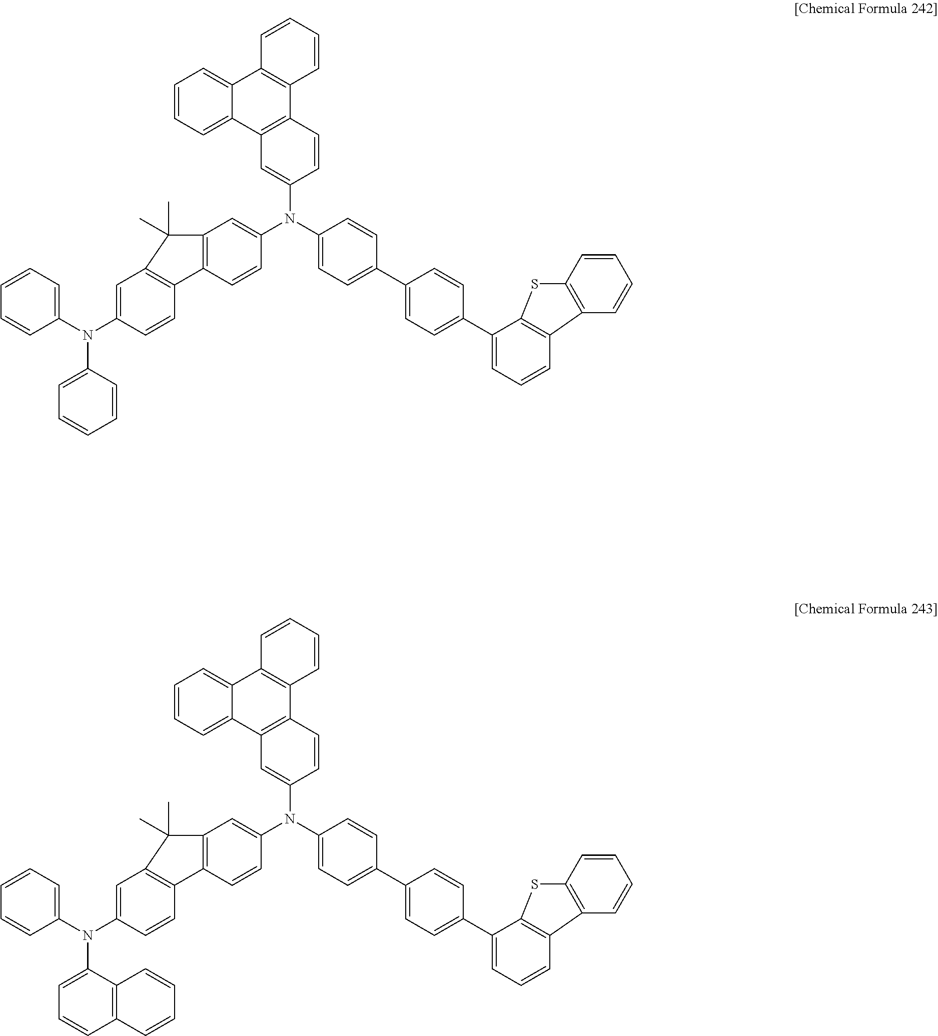 [SMILES: CC1(C)c2cc(N(c3ccccc3)c3ccccc3)ccc2-c2ccc(N(c3ccc(-c4ccc(-c5cccc6c5sc5ccccc56)cc4)cc3)c3ccc4c5ccccc5c5ccccc5c4c3)cc21 ; CC1(C)c2cc(N(c3ccc(-c4ccc(-c5cccc6c5sc5ccccc56)cc4)cc3)c3ccc4c5ccccc5c5ccccc5c4c3)ccc2-c2ccc(N(c3ccccc3)c3cccc4ccccc34)cc21]

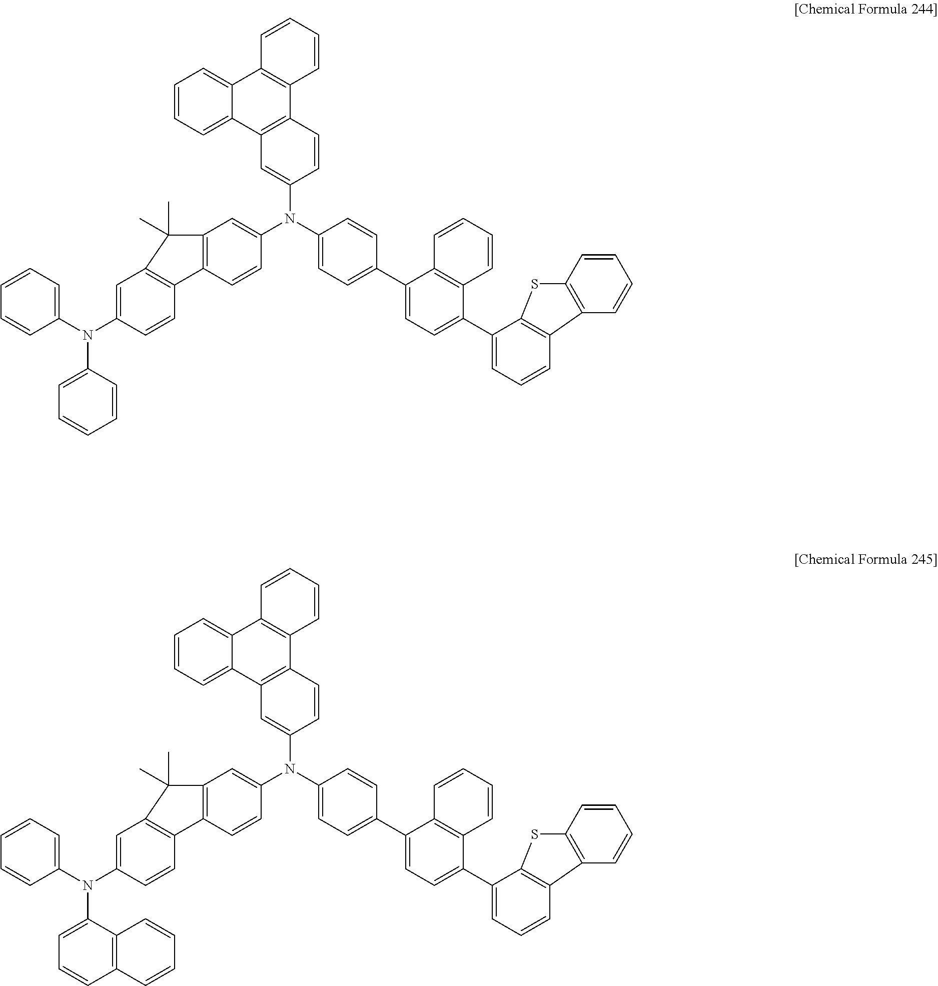 [SMILES: CC1(C)c2cc(N(c3ccccc3)c3ccccc3)ccc2-c2ccc(N(c3ccc(-c4ccc(-c5cccc6c5sc5ccccc56)c5ccccc45)cc3)c3ccc4c5ccccc5c5ccccc5c4c3)cc21 ; CC1(C)c2cc(N(c3ccc(-c4ccc(-c5cccc6c5sc5ccccc56)c5ccccc45)cc3)c3ccc4c5ccccc5c5ccccc5c4c3)ccc2-c2ccc(N(c3ccccc3)c3cccc4ccccc34)cc21]

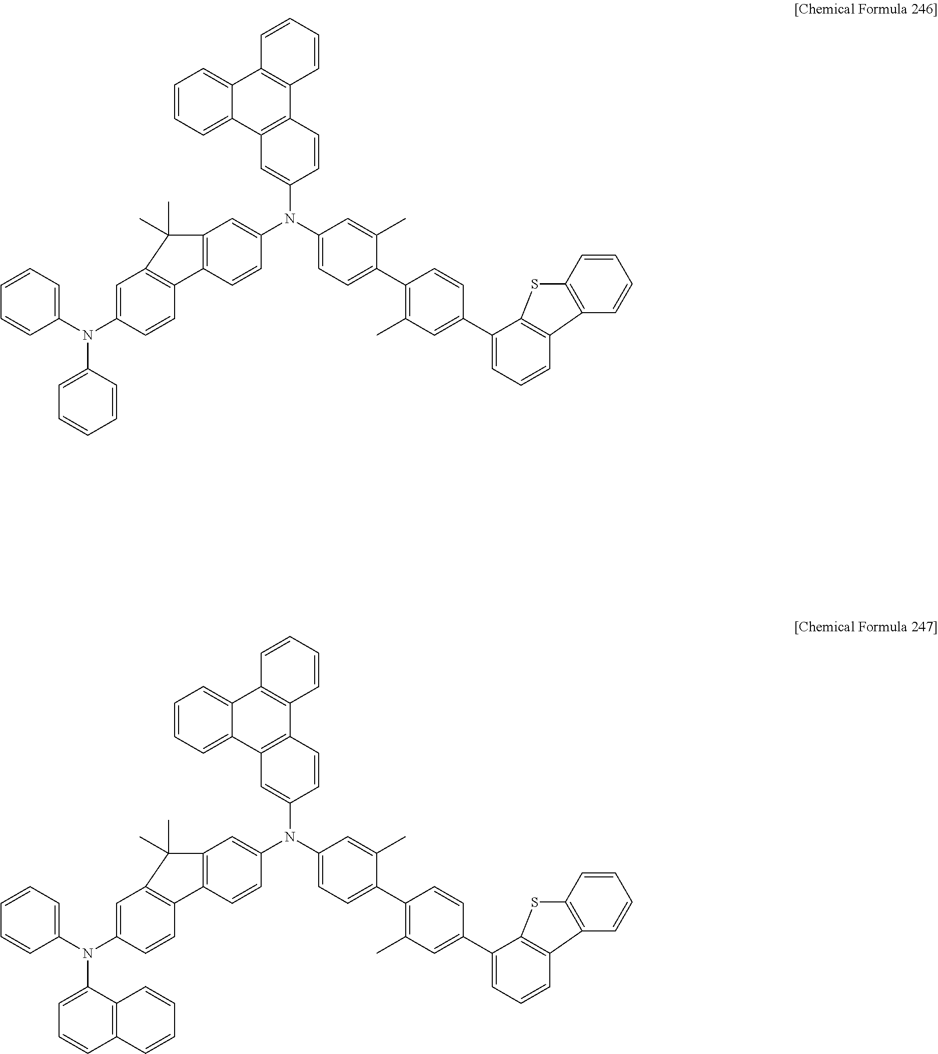 [SMILES: Cc1cc(-c2cccc3c2sc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2cccc3c2sc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

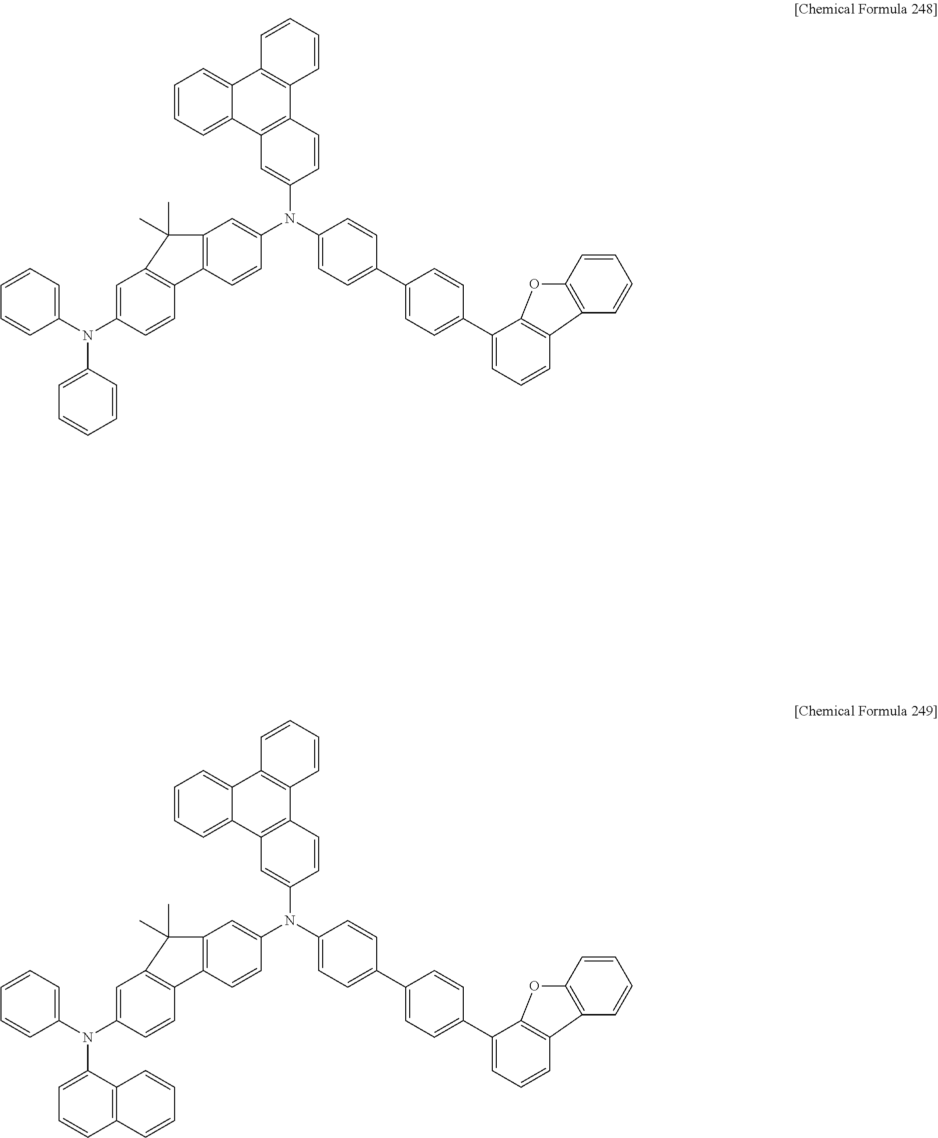 [SMILES: CC1(C)c2cc(N(c3ccccc3)c3ccccc3)ccc2-c2ccc(N(c3ccc(-c4ccc(-c5cccc6c5oc5ccccc56)cc4)cc3)c3ccc4c5ccccc5c5ccccc5c4c3)cc21 ; CC1(C)c2cc(N(c3ccc(-c4ccc(-c5cccc6c5oc5ccccc56)cc4)cc3)c3ccc4c5ccccc5c5ccccc5c4c3)ccc2-c2ccc(N(c3ccccc3)c3cccc4ccccc34)cc21]

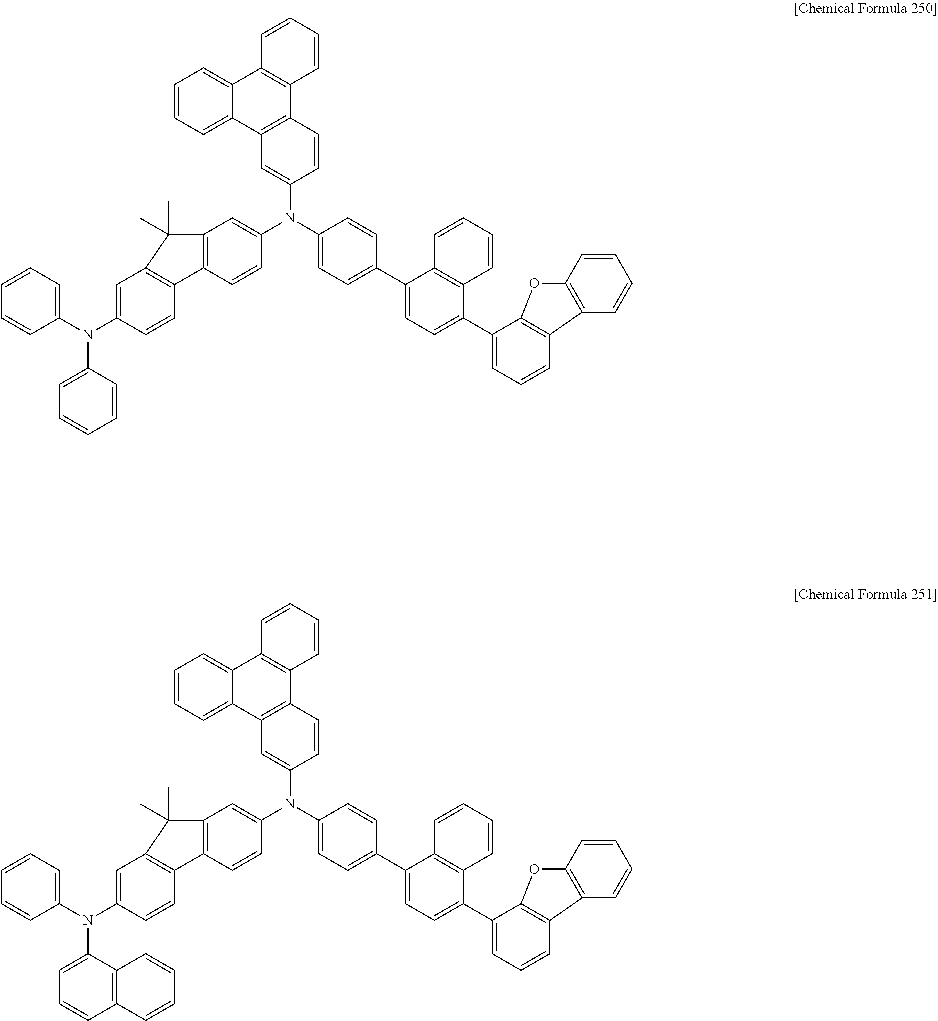 [SMILES: CC1(C)c2cc(N(c3ccccc3)c3ccccc3)ccc2-c2ccc(N(c3ccc(-c4ccc(-c5cccc6c5oc5ccccc56)c5ccccc45)cc3)c3ccc4c5ccccc5c5ccccc5c4c3)cc21 ; CC1(C)c2cc(N(c3ccc(-c4ccc(-c5cccc6c5oc5ccccc56)c5ccccc45)cc3)c3ccc4c5ccccc5c5ccccc5c4c3)ccc2-c2ccc(N(c3ccccc3)c3cccc4ccccc34)cc21]

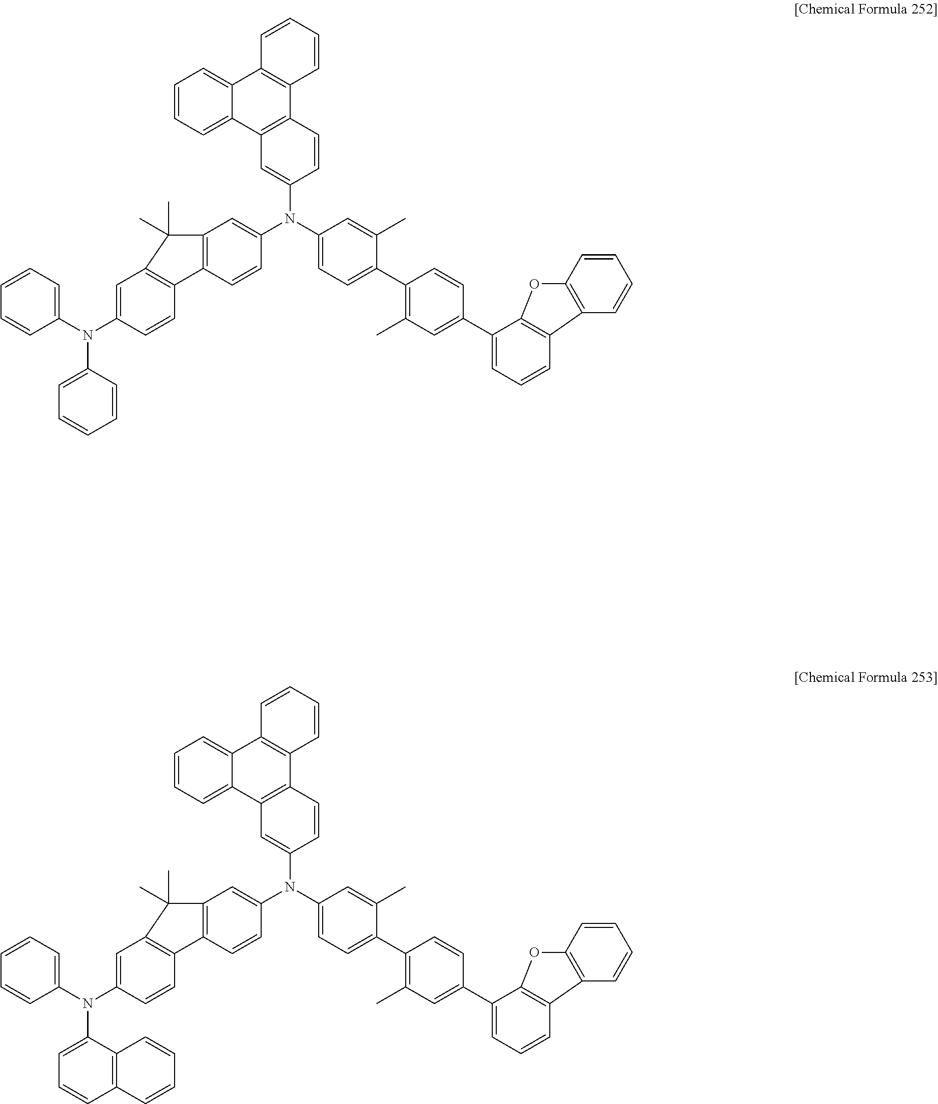 [SMILES: Cc1cc(-c2cccc3c2oc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2cccc3c2oc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

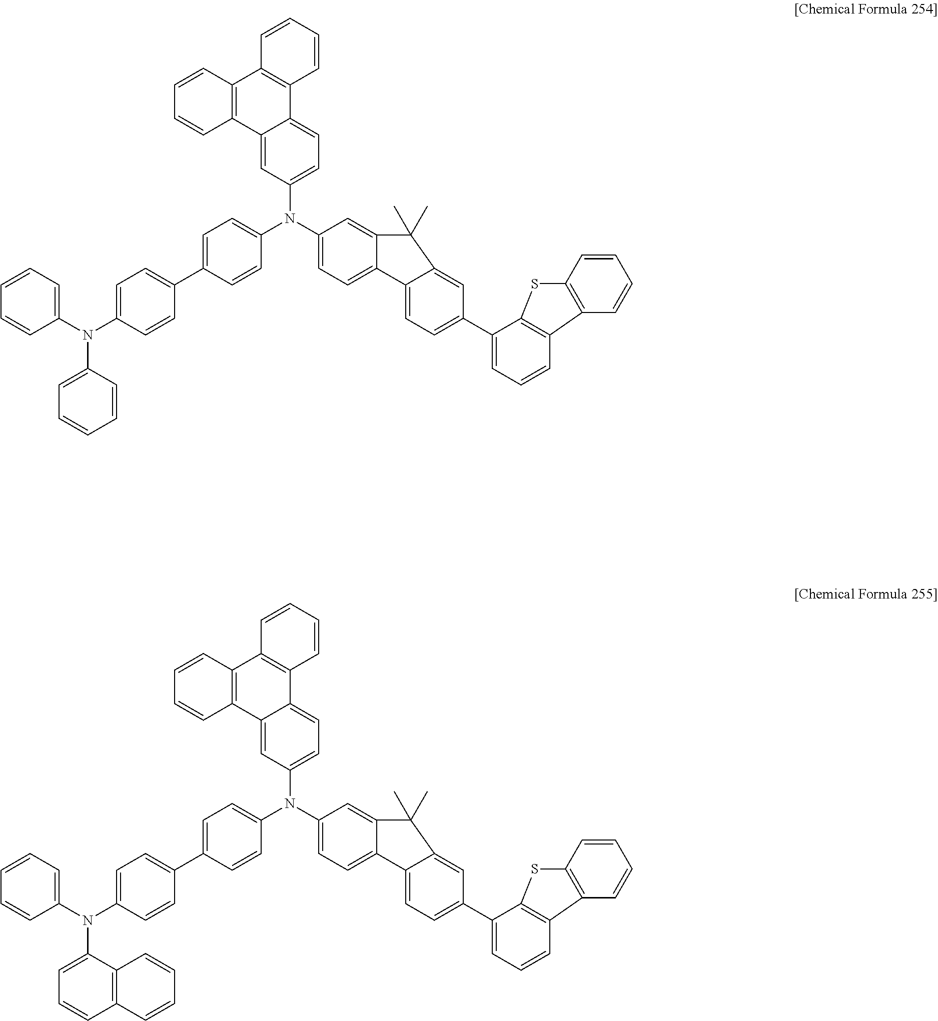 [SMILES: CC1(C)c2cc(-c3cccc4c3sc3ccccc34)ccc2-c2ccc(N(c3ccc(-c4ccc(N(c5ccccc5)c5ccccc5)cc4)cc3)c3ccc4c5ccccc5c5ccccc5c4c3)cc21 ; CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21]

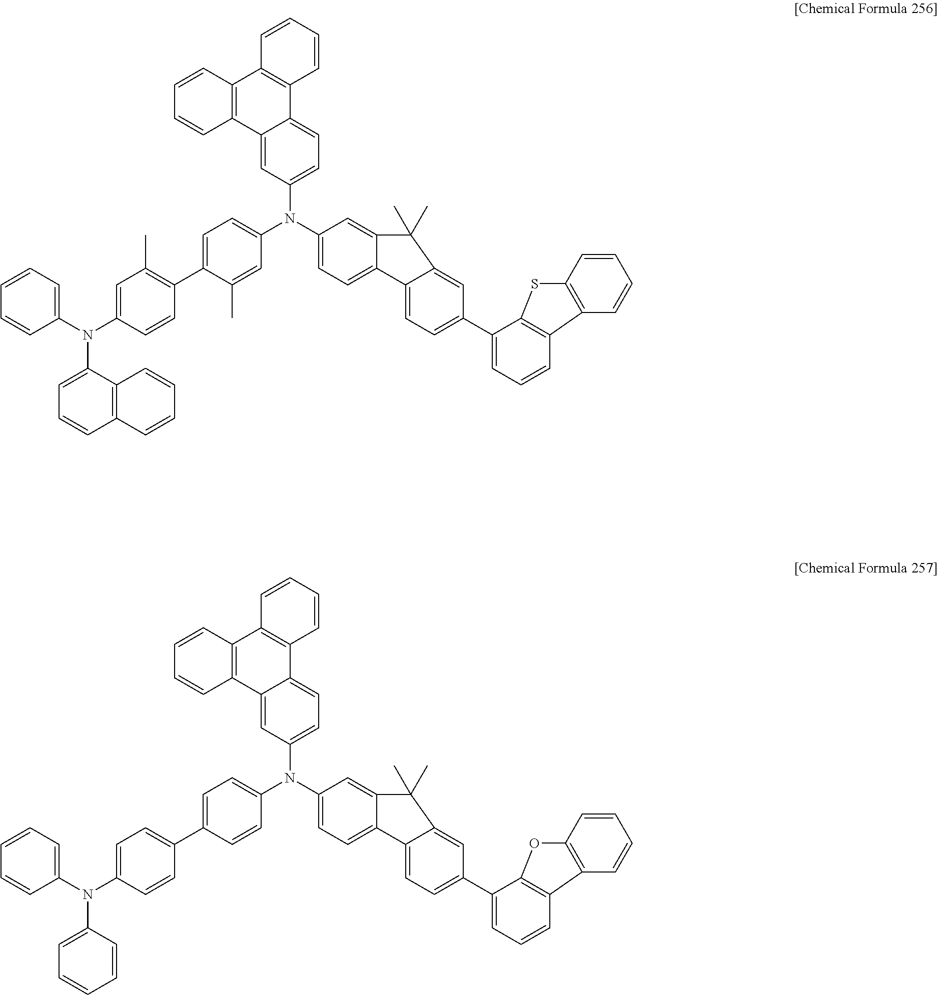 [SMILES: Cc1cc(N(c2ccc3c(c2)C(C)(C)c2cc(-c4cccc5c4sc4ccccc45)ccc2-3)c2ccc3c4ccccc4c4ccccc4c3c2)ccc1-c1ccc(N(c2ccccc2)c2cccc3ccccc23)cc1C ; CC1(C)c2cc(-c3cccc4c3oc3ccccc34)ccc2-c2ccc(N(c3ccc(-c4ccc(N(c5ccccc5)c5ccccc5)cc4)cc3)c3ccc4c5ccccc5c5ccccc5c4c3)cc21]

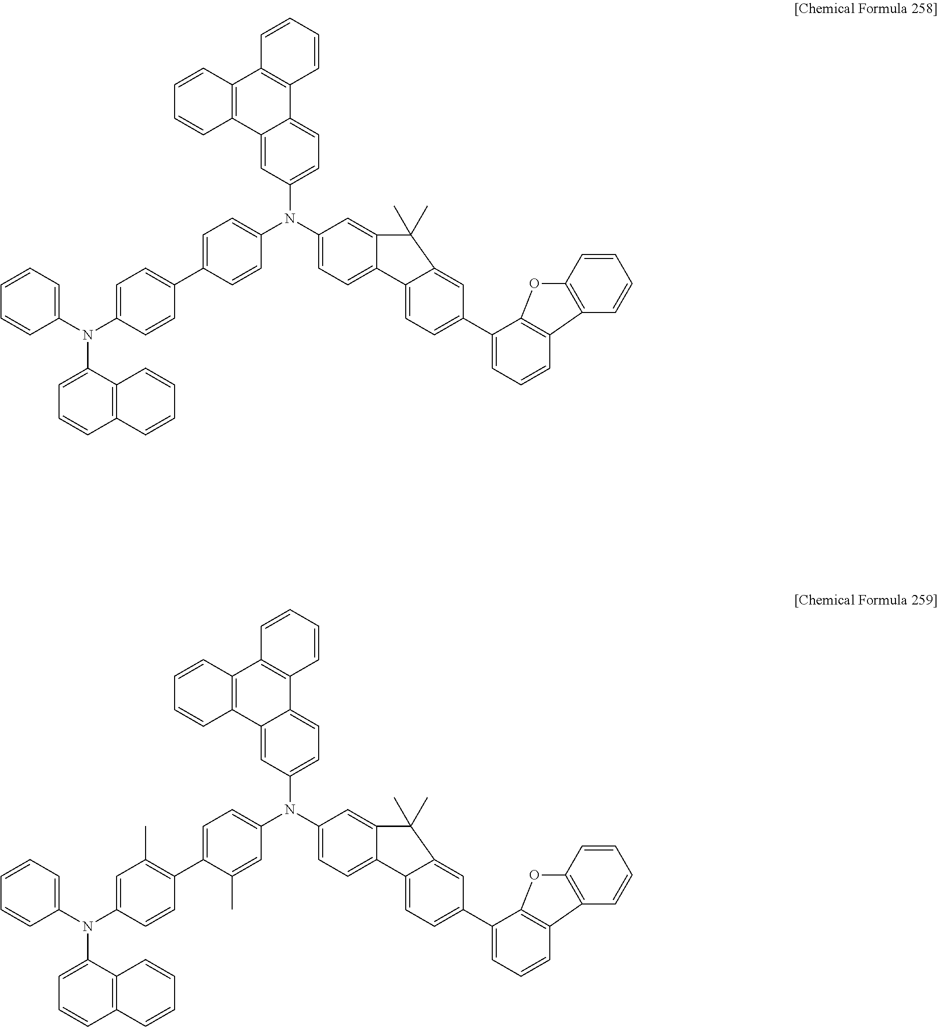 [SMILES: CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21 ; Cc1cc(N(c2ccc3c(c2)C(C)(C)c2cc(-c4cccc5c4oc4ccccc45)ccc2-3)c2ccc3c4ccccc4c4ccccc4c3c2)ccc1-c1ccc(N(c2ccccc2)c2cccc3ccccc23)cc1C]

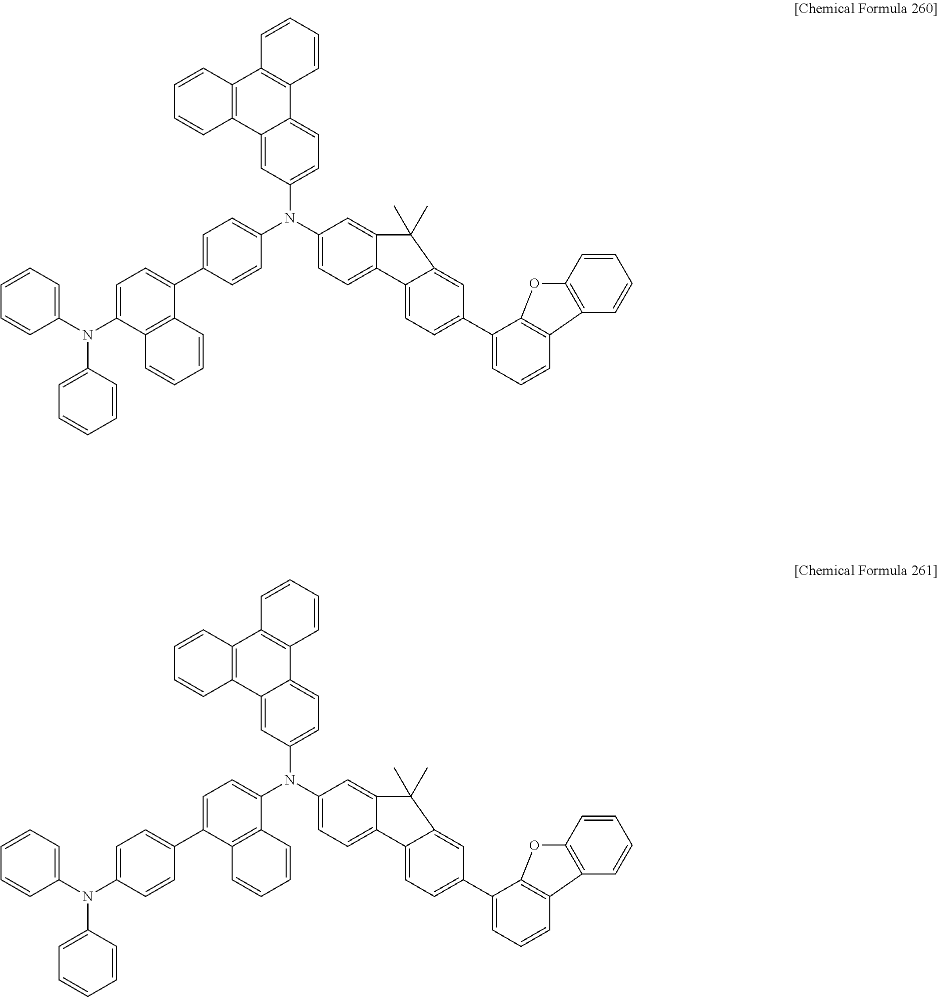 [SMILES: CC1(C)c2cc(-c3cccc4c3oc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21 ; CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21]

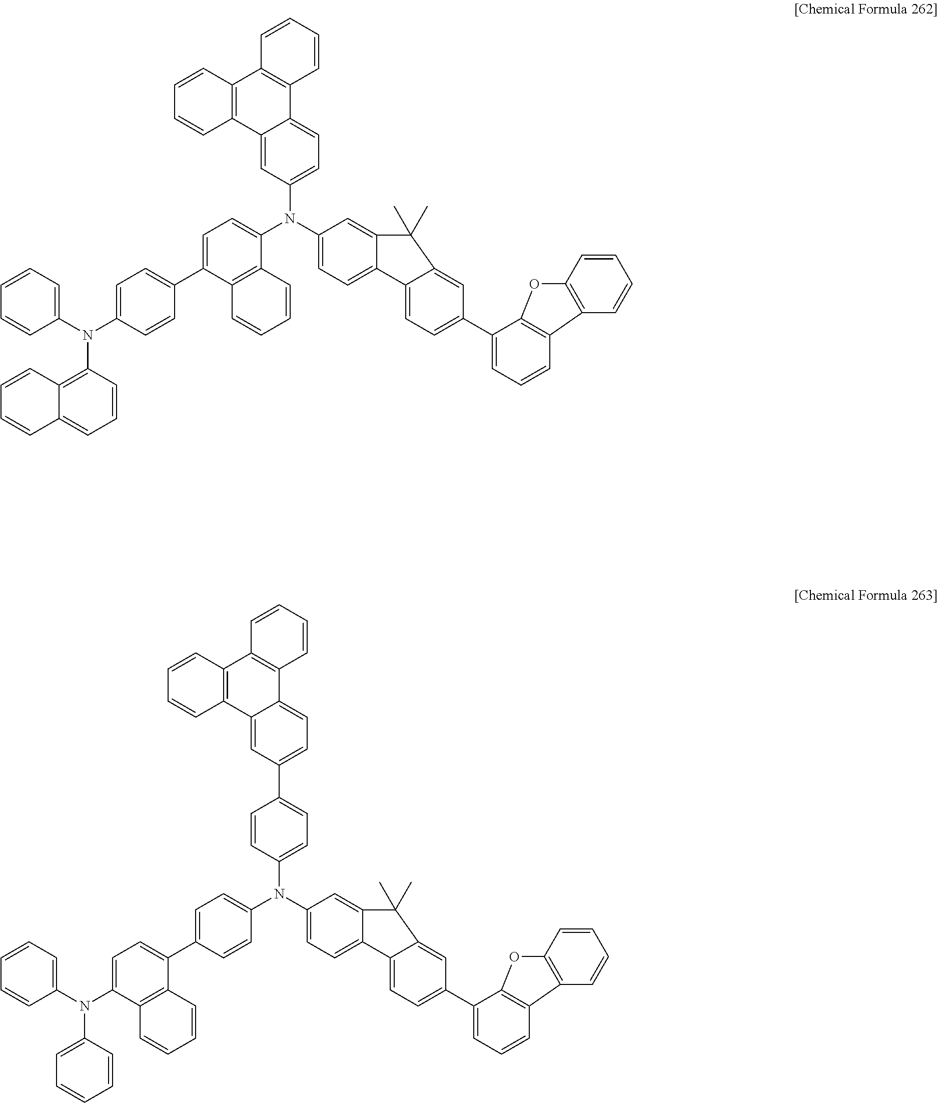 [SMILES: CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21 ; CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21]

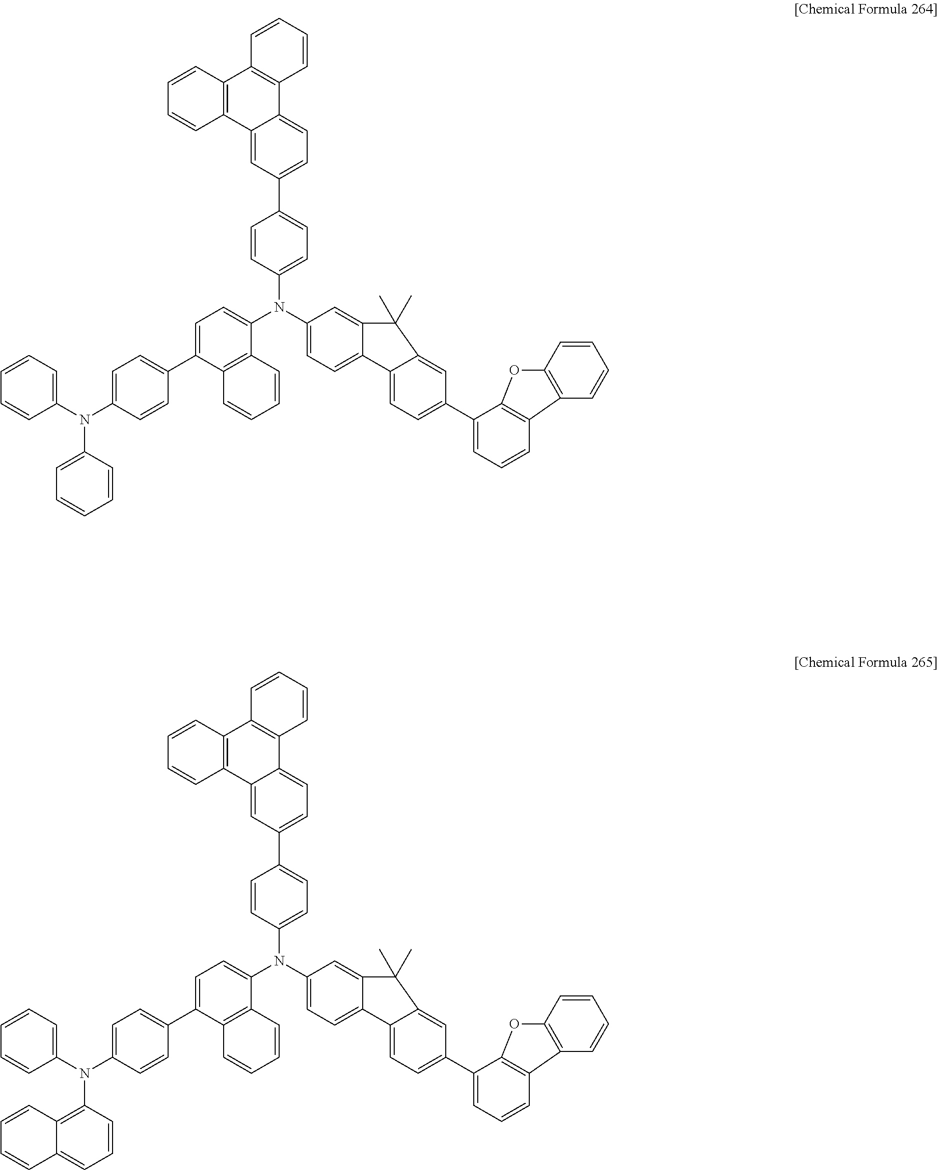 [SMILES: CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21 ; CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21]

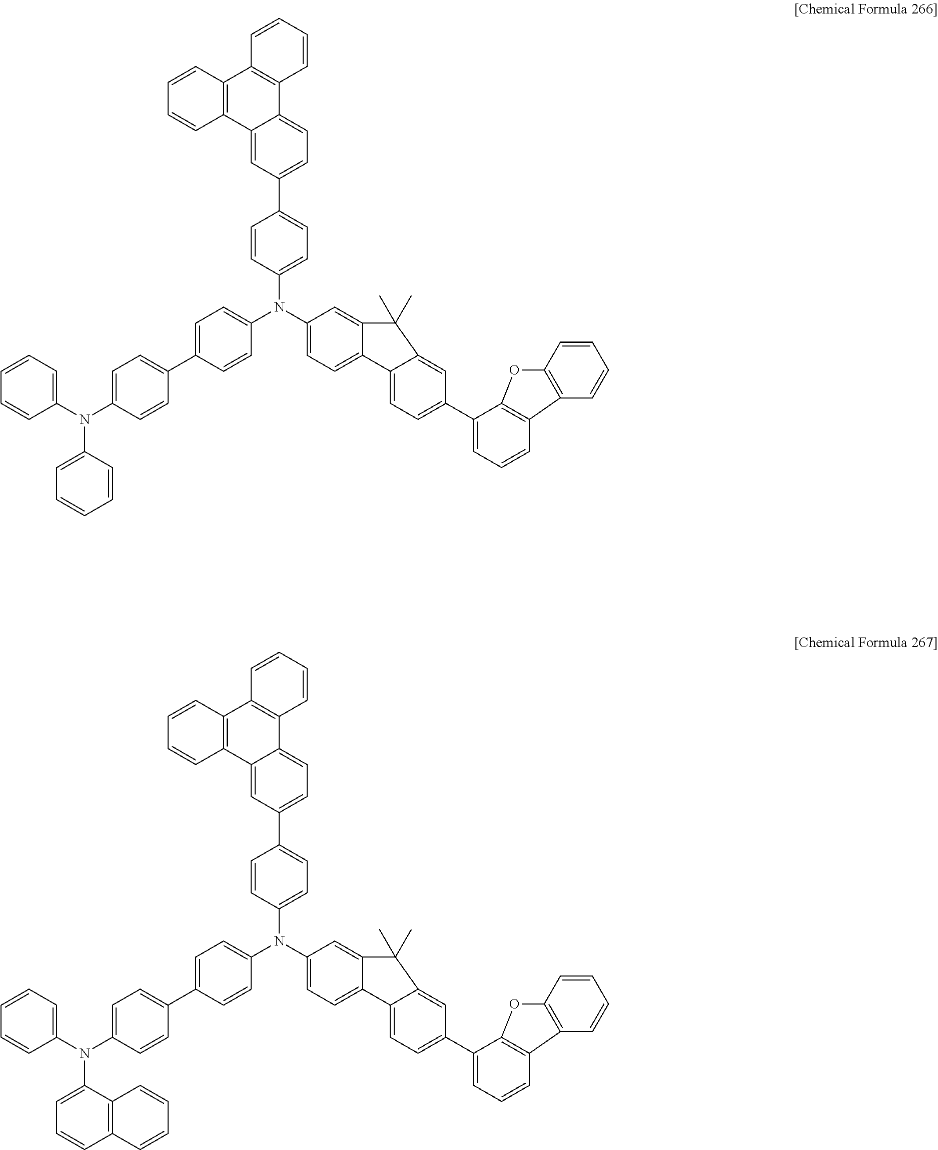 [SMILES: CC1(C)c2cc(-c3cccc4c3oc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21 ; CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21]

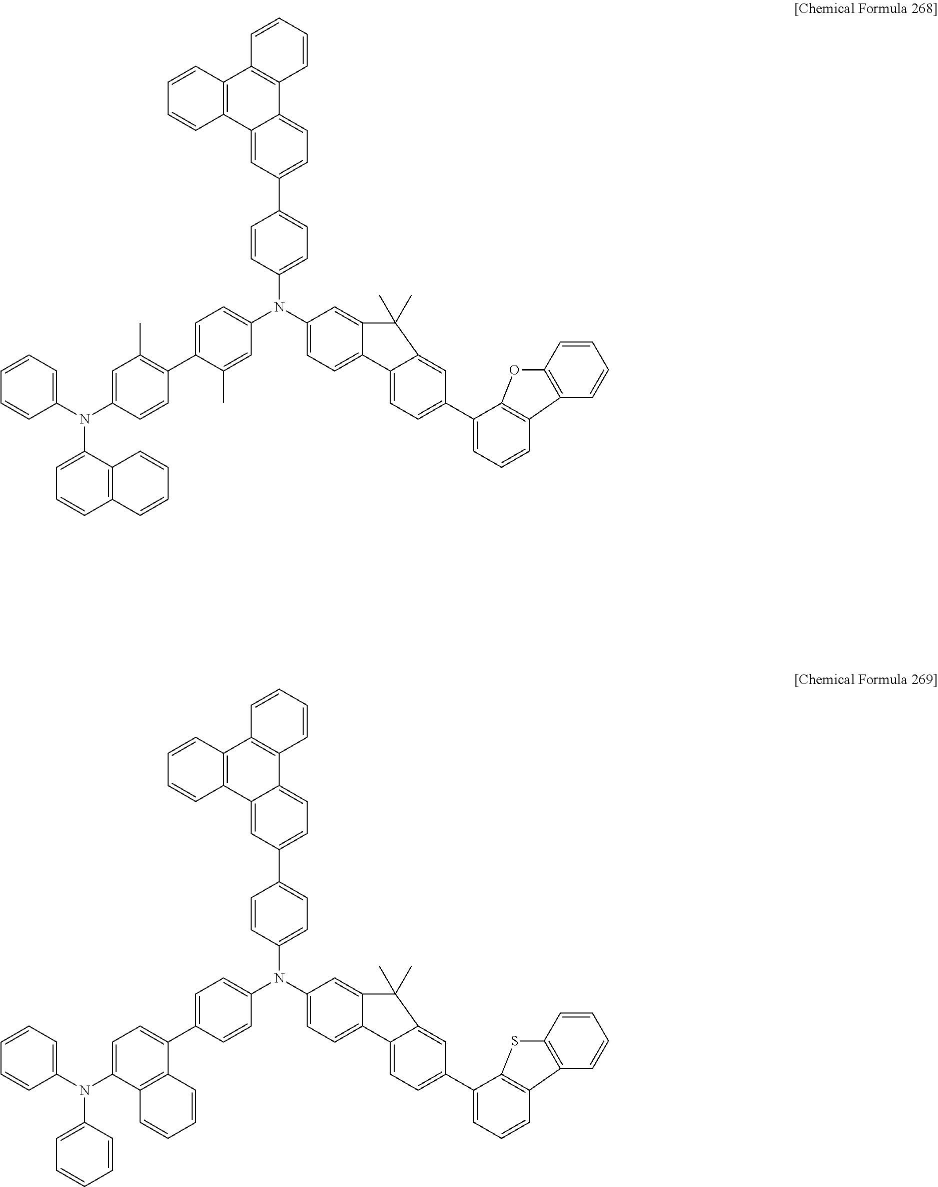 [SMILES: Cc1cc(N(c2ccc(-c3ccc4c5ccccc5c5ccccc5c4c3)cc2)c2ccc3c(c2)C(C)(C)c2cc(-c4cccc5c4oc4ccccc45)ccc2-3)ccc1-c1ccc(N(c2ccccc2)c2cccc3ccccc23)cc1C ; CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21]

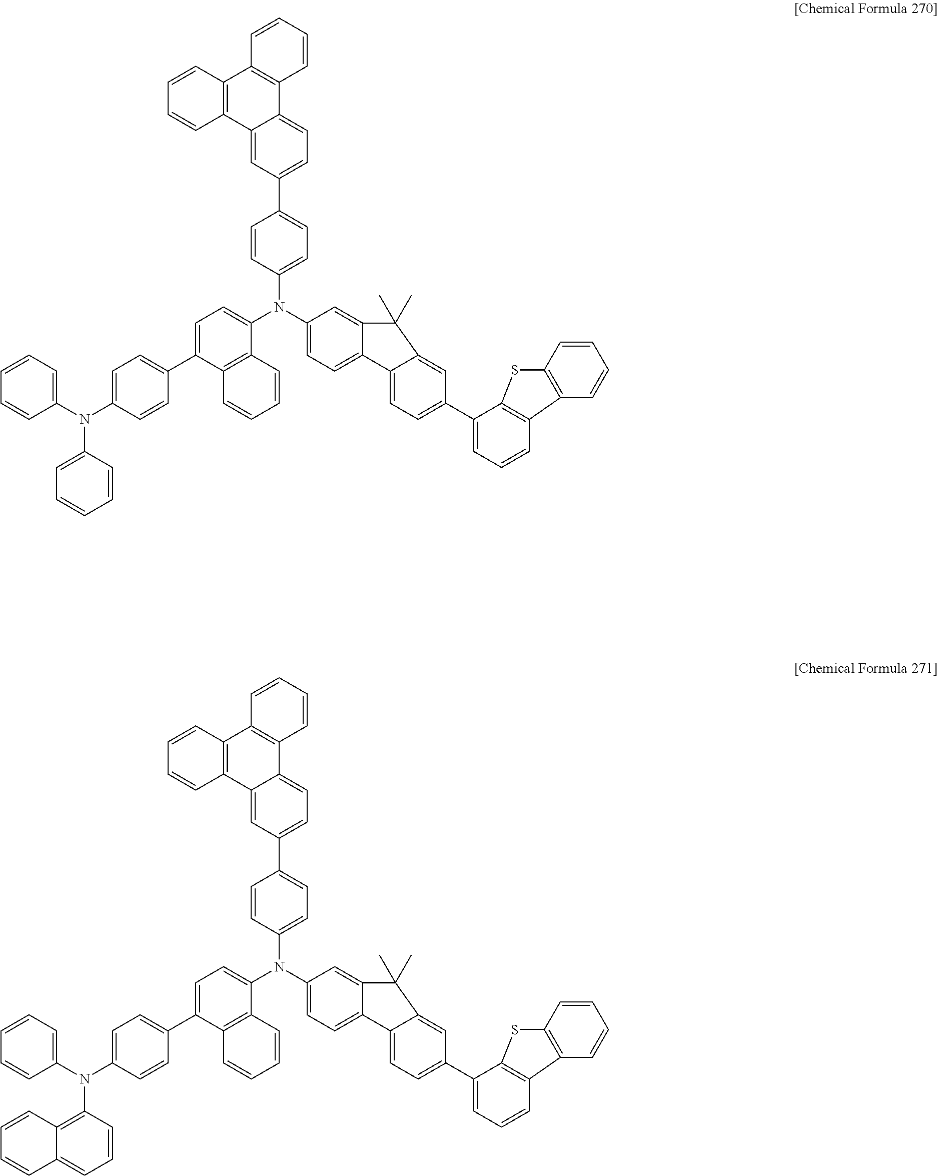 [SMILES: CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21 ; CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21]

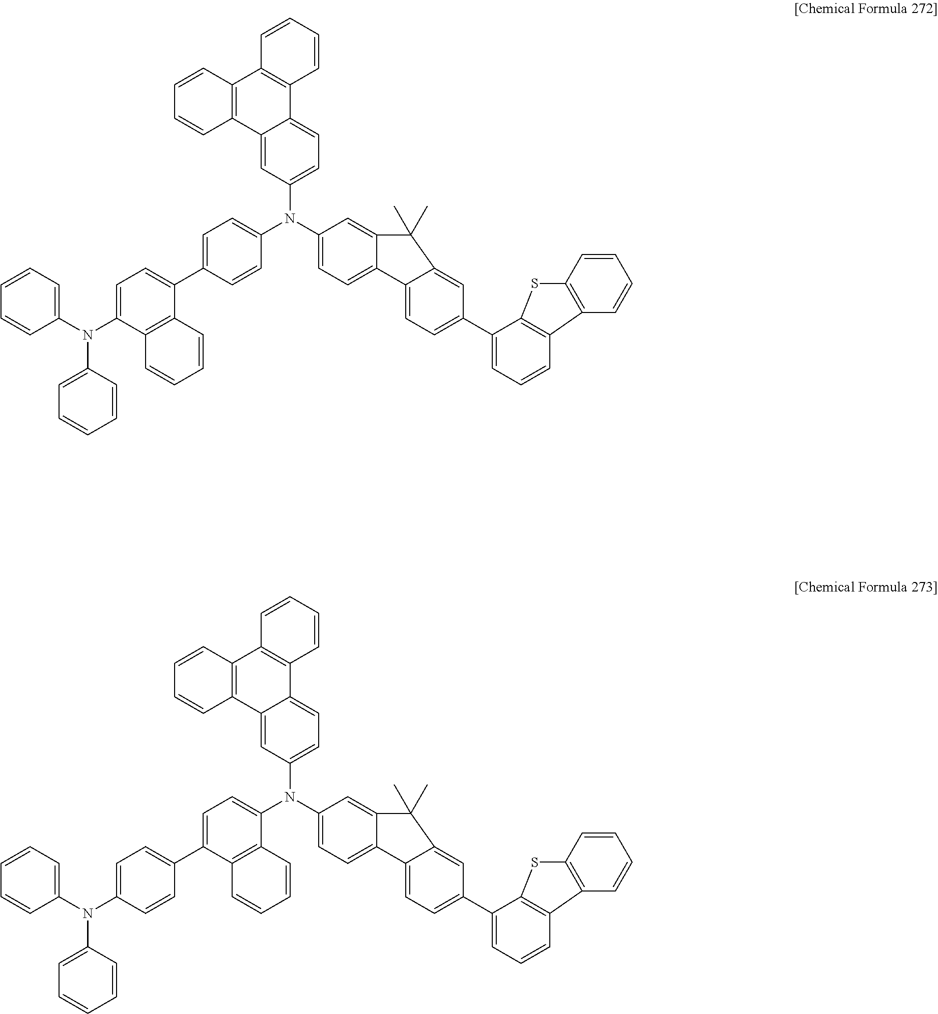 [SMILES: CC1(C)c2cc(-c3cccc4c3sc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21 ; CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21]

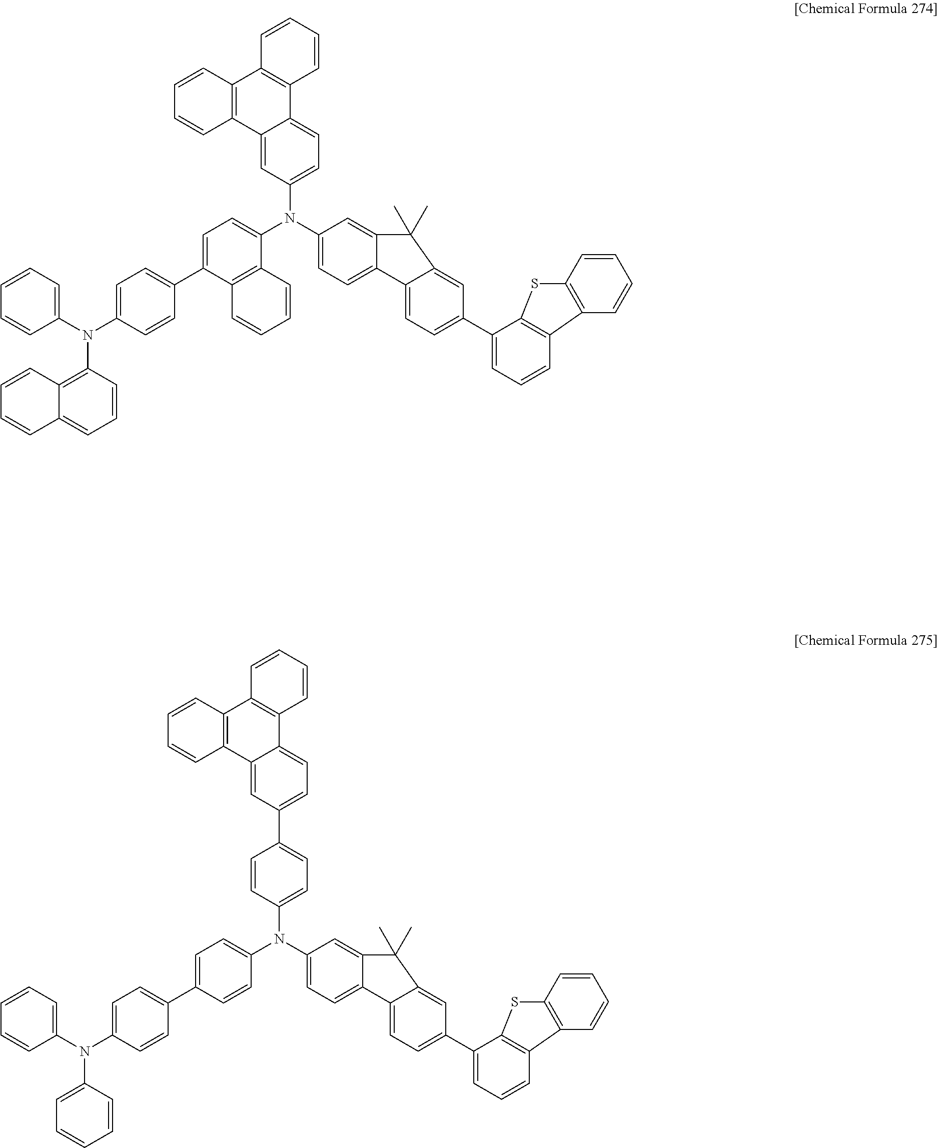 [SMILES: CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21 ; CC1(C)c2cc(-c3cccc4c3sc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21]

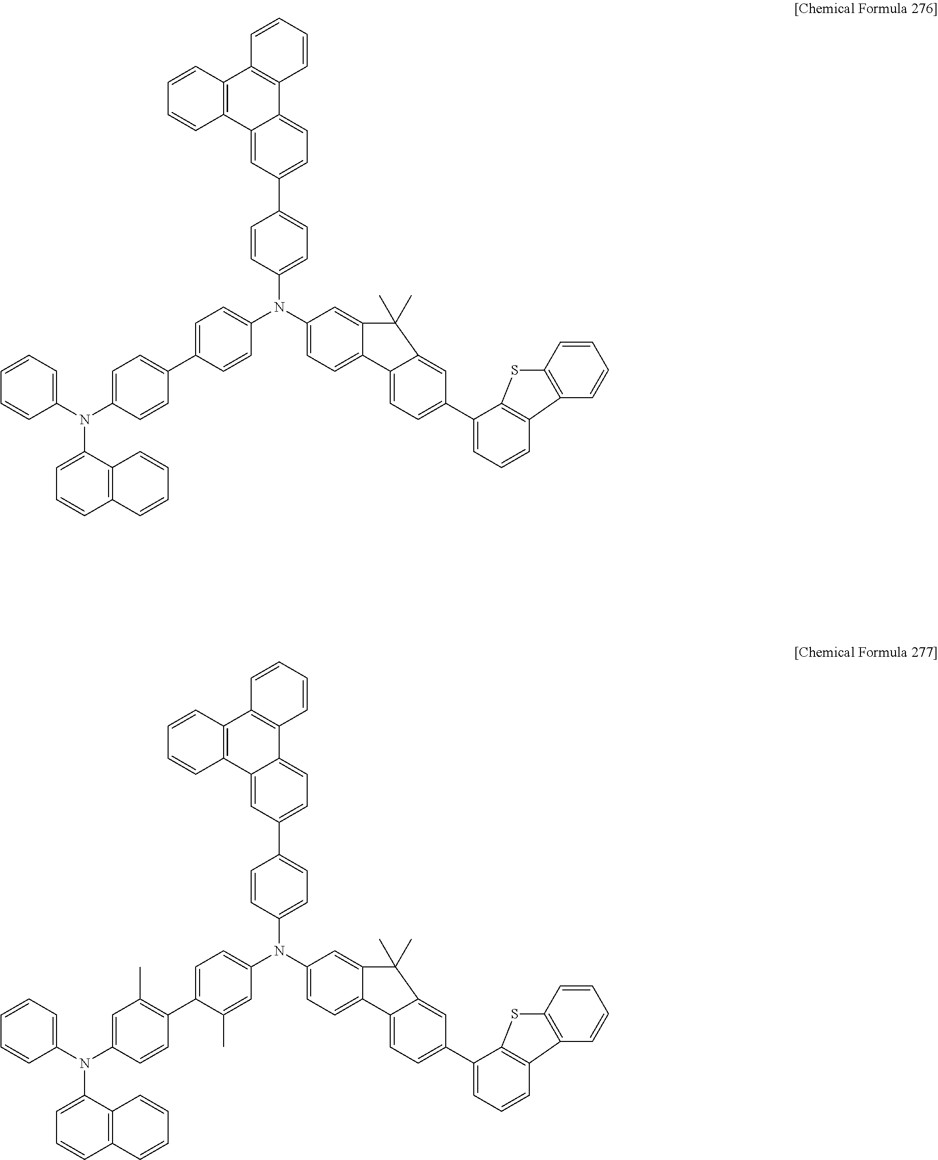 [SMILES: CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21 ; Cc1cc(N(c2ccc(-c3ccc4c5ccccc5c5ccccc5c4c3)cc2)c2ccc3c(c2)C(C)(C)c2cc(-c4cccc5c4sc4ccccc45)ccc2-3)ccc1-c1ccc(N(c2ccccc2)c2cccc3ccccc23)cc1C]

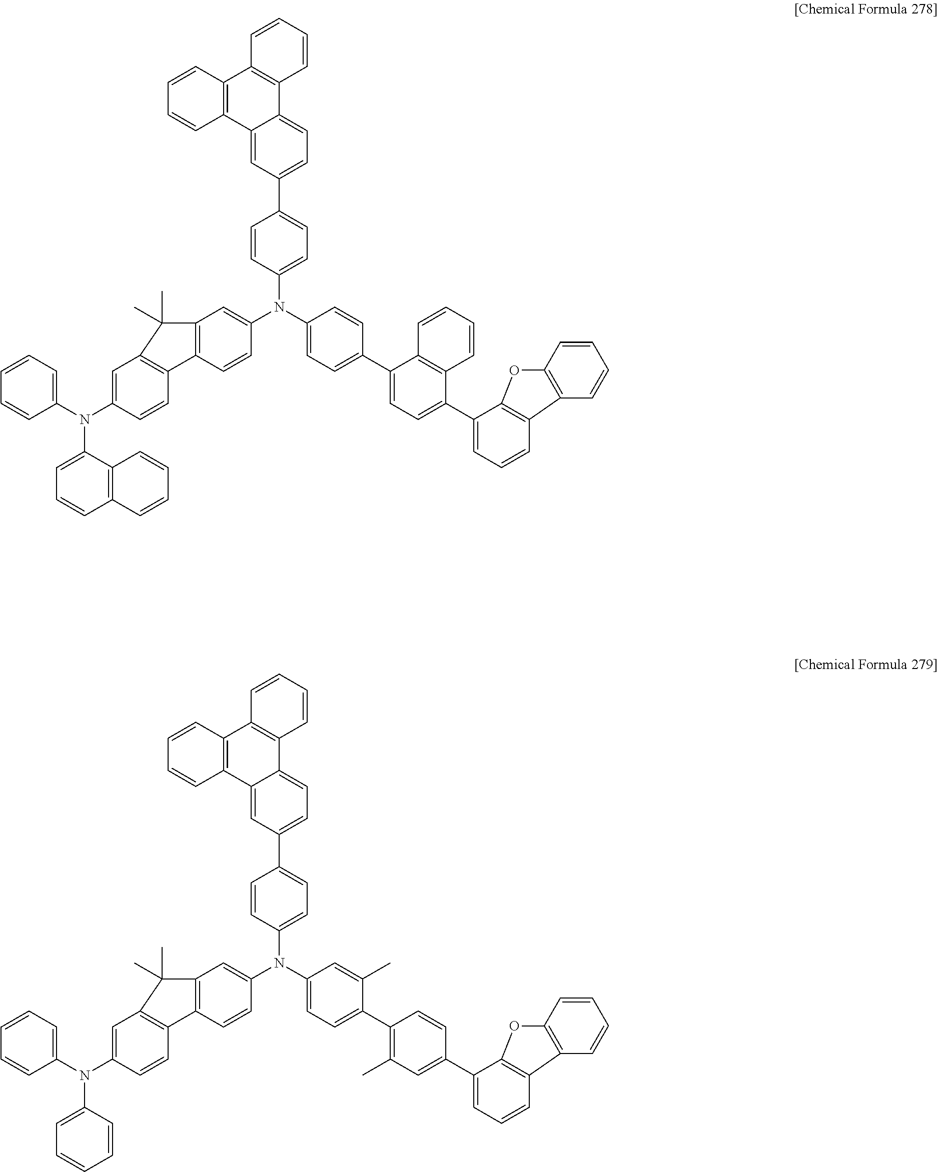 [SMILES: CC1(C)c2cc(N(c3ccc(-c4ccc5c6ccccc6c6ccccc6c5c4)cc3)c3ccc(-c4ccc(-c5cccc6c5oc5ccccc56)c5ccccc45)cc3)ccc2-c2ccc(N(c3ccccc3)c3cccc4ccccc34)cc21 ; Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

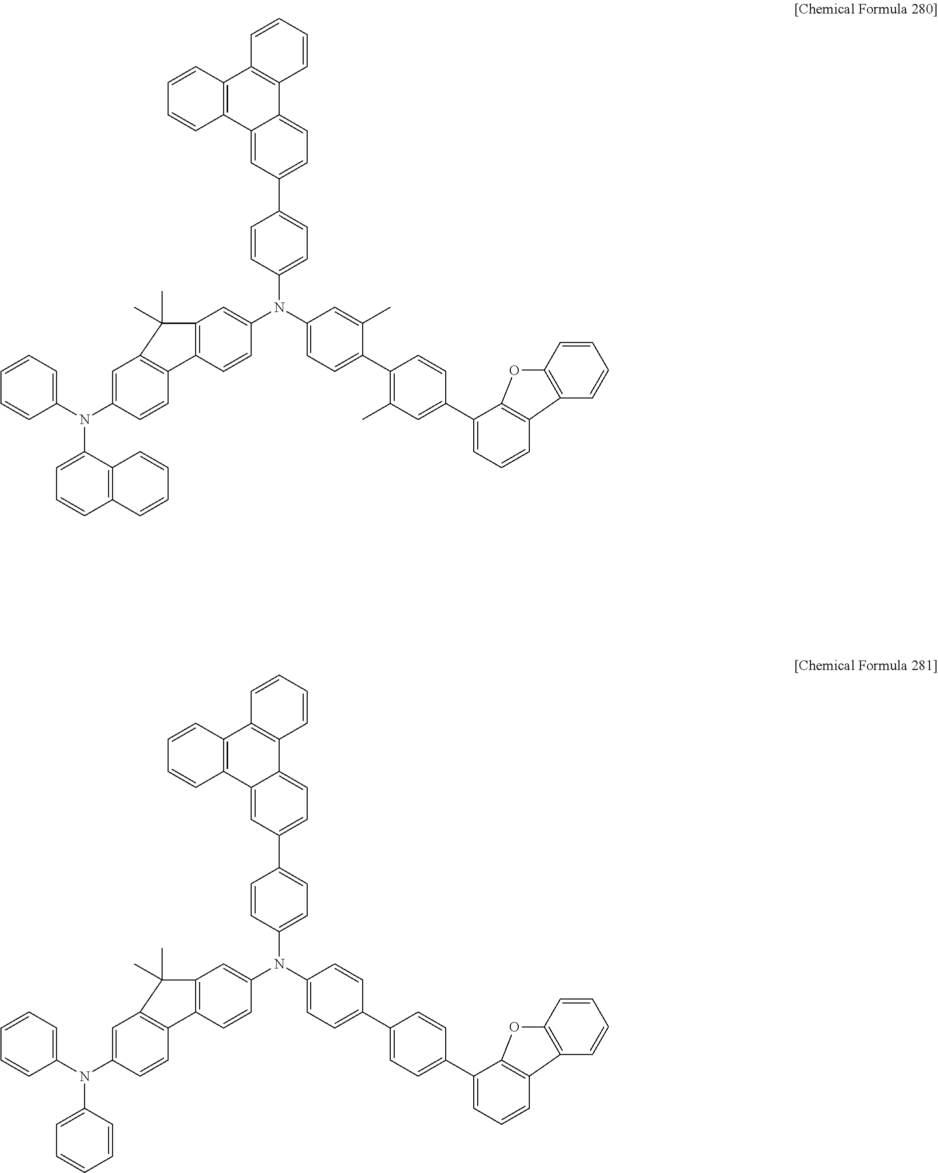 [SMILES: Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5cccc6c5oc5ccccc56)cc4)cc3)c3ccc(-c4ccc5c6ccccc6c6ccccc6c5c4)cc3)cc21]

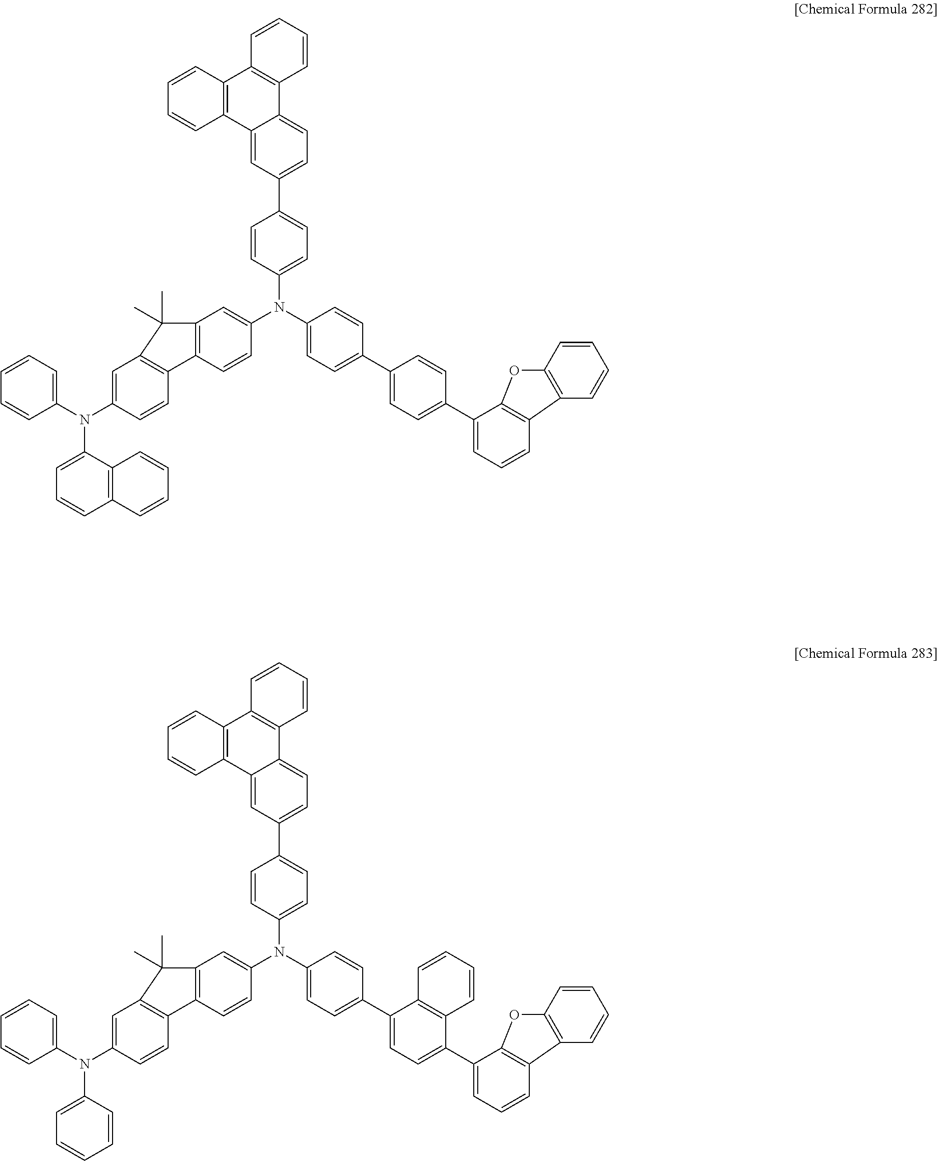 [SMILES: CC1(C)c2cc(N(c3ccc(-c4ccc(-c5cccc6c5oc5ccccc56)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5oc5ccccc56)c5ccccc45)cc3)cc21]

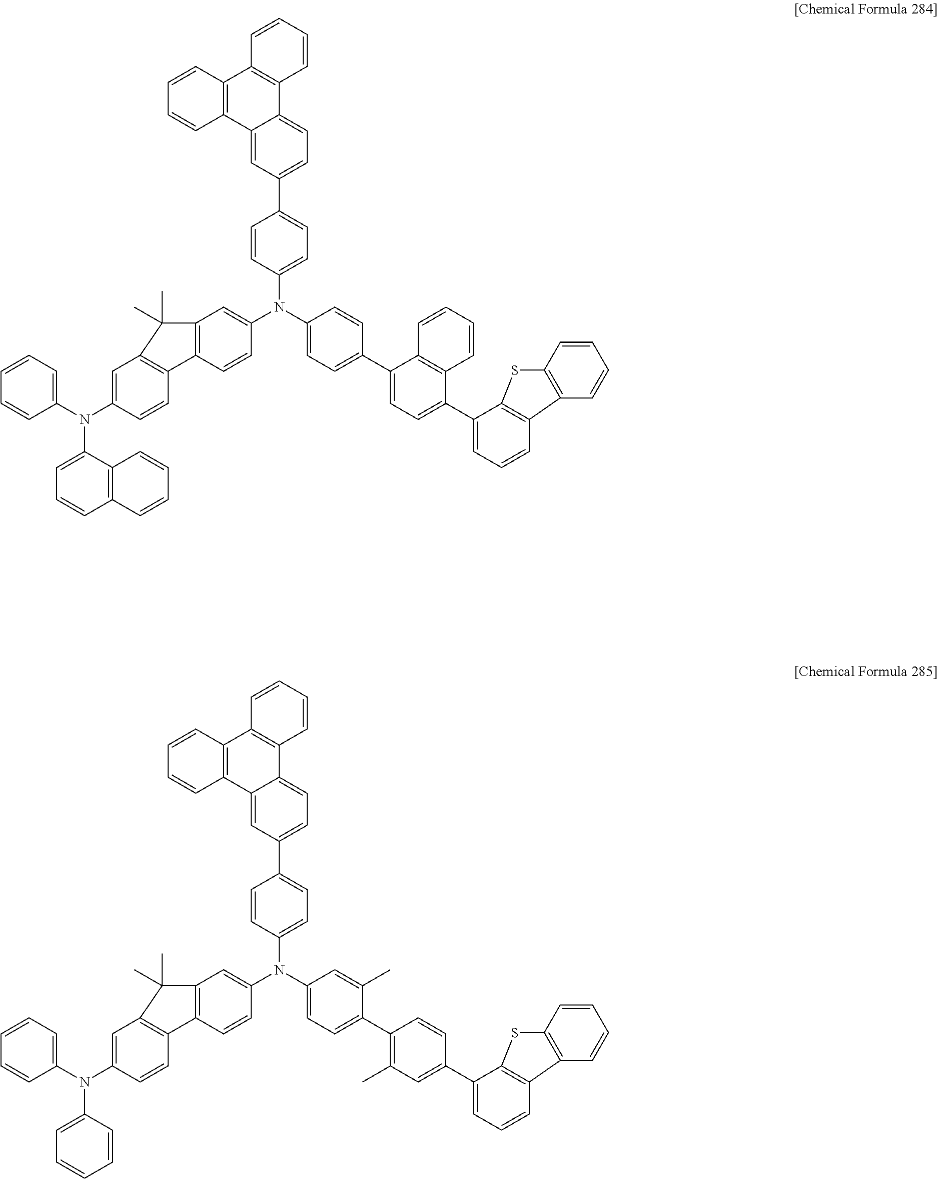 [SMILES: CC1(C)c2cc(N(c3ccc(-c4ccc5c6ccccc6c6ccccc6c5c4)cc3)c3ccc(-c4ccc(-c5cccc6c5sc5ccccc56)c5ccccc45)cc3)ccc2-c2ccc(N(c3ccccc3)c3cccc4ccccc34)cc21 ; Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

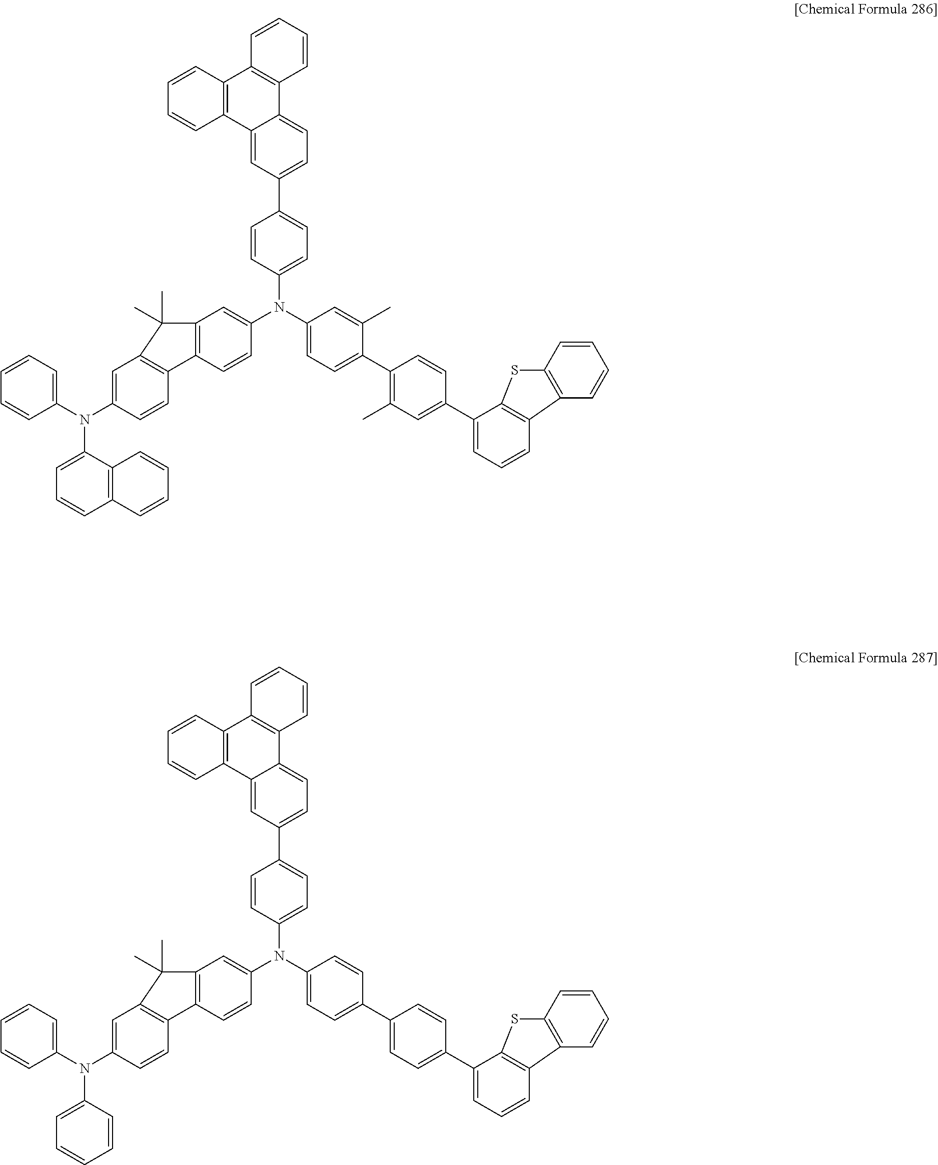 [SMILES: Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5cccc6c5sc5ccccc56)cc4)cc3)c3ccc(-c4ccc5c6ccccc6c6ccccc6c5c4)cc3)cc21]

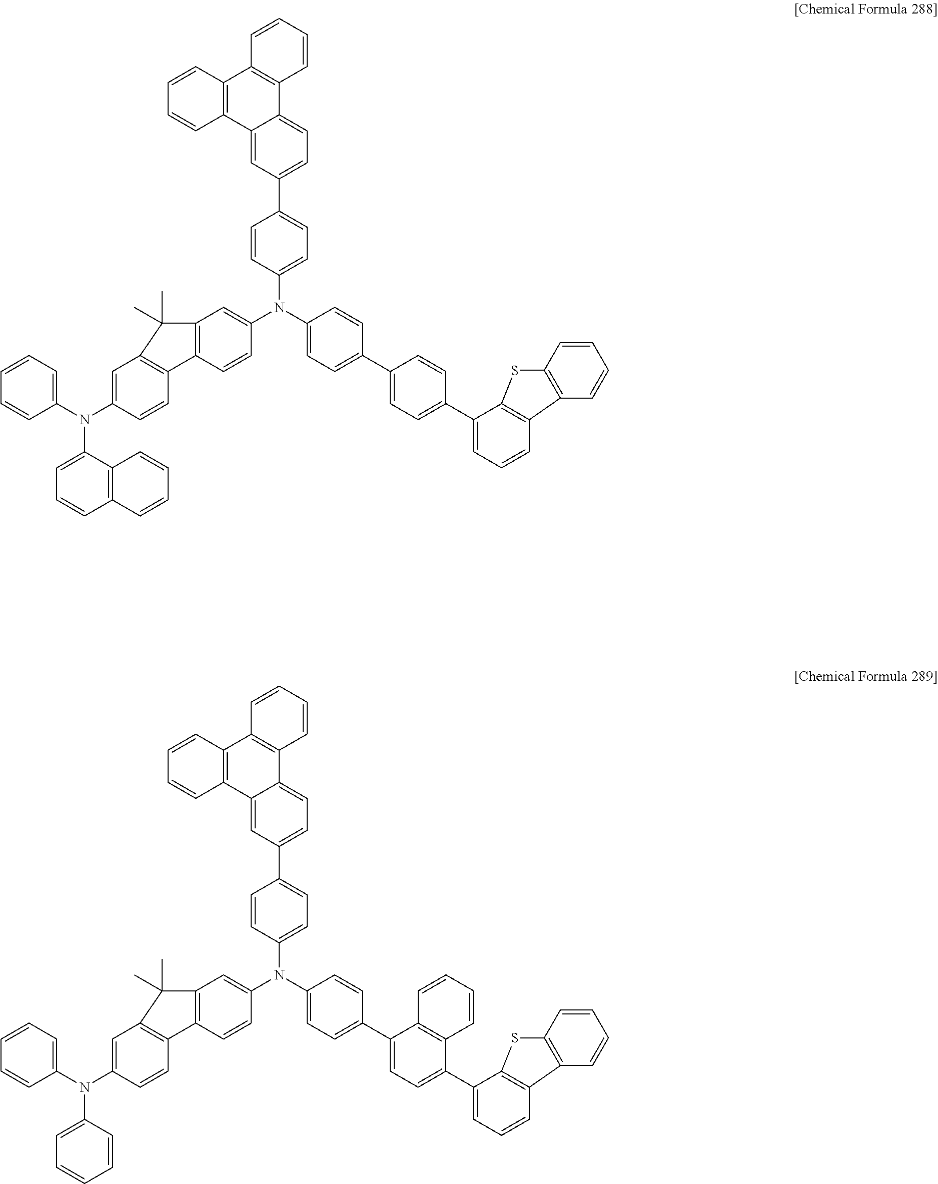 [SMILES: CC1(C)c2cc(N(c3ccc(-c4ccc(-c5cccc6c5sc5ccccc56)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5sc5ccccc56)c5ccccc45)cc3)cc21]

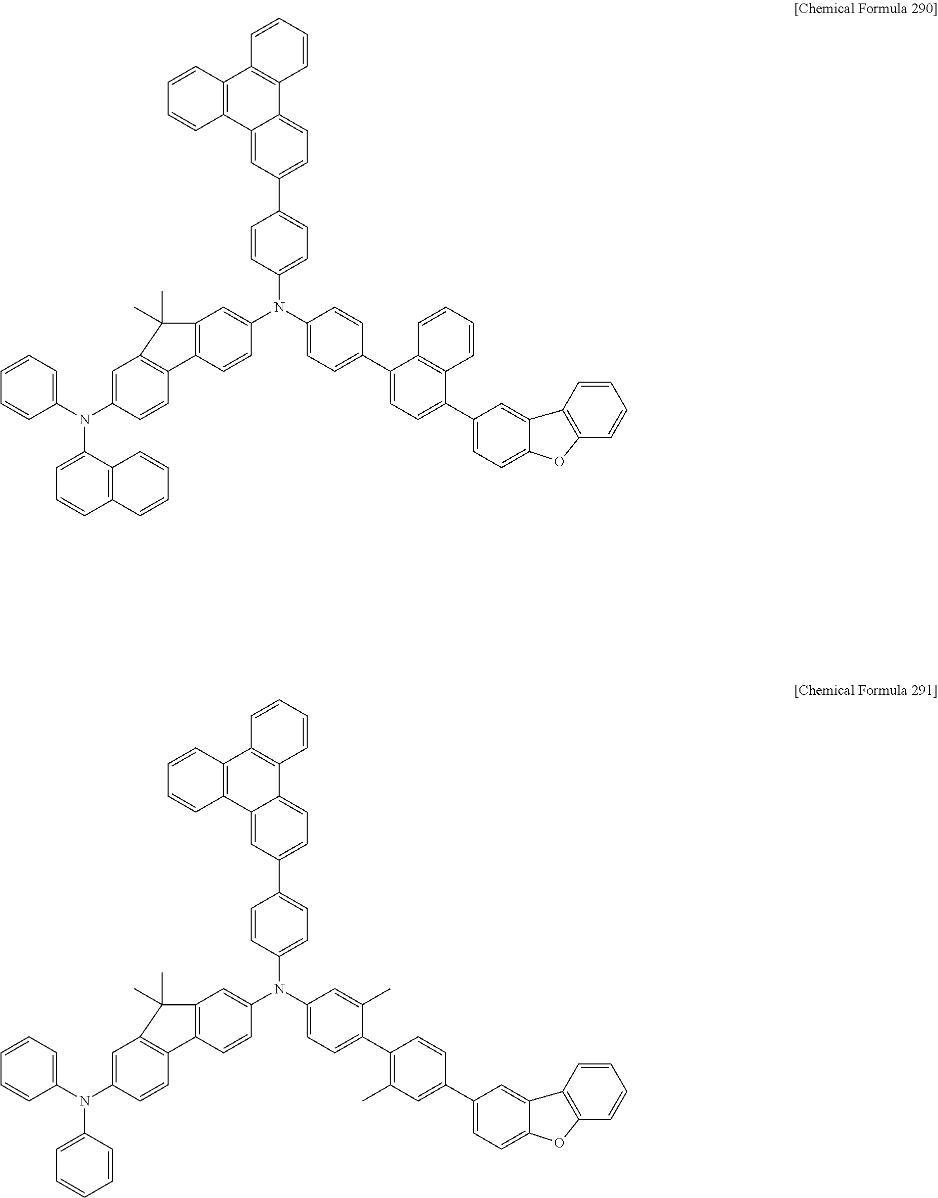 [SMILES: CC1(C)c2cc(N(c3ccc(-c4ccc5c6ccccc6c6ccccc6c5c4)cc3)c3ccc(-c4ccc(-c5ccc6oc7ccccc7c6c5)c5ccccc45)cc3)ccc2-c2ccc(N(c3ccccc3)c3cccc4ccccc34)cc21 ; Cc1cc(-c2ccc3oc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

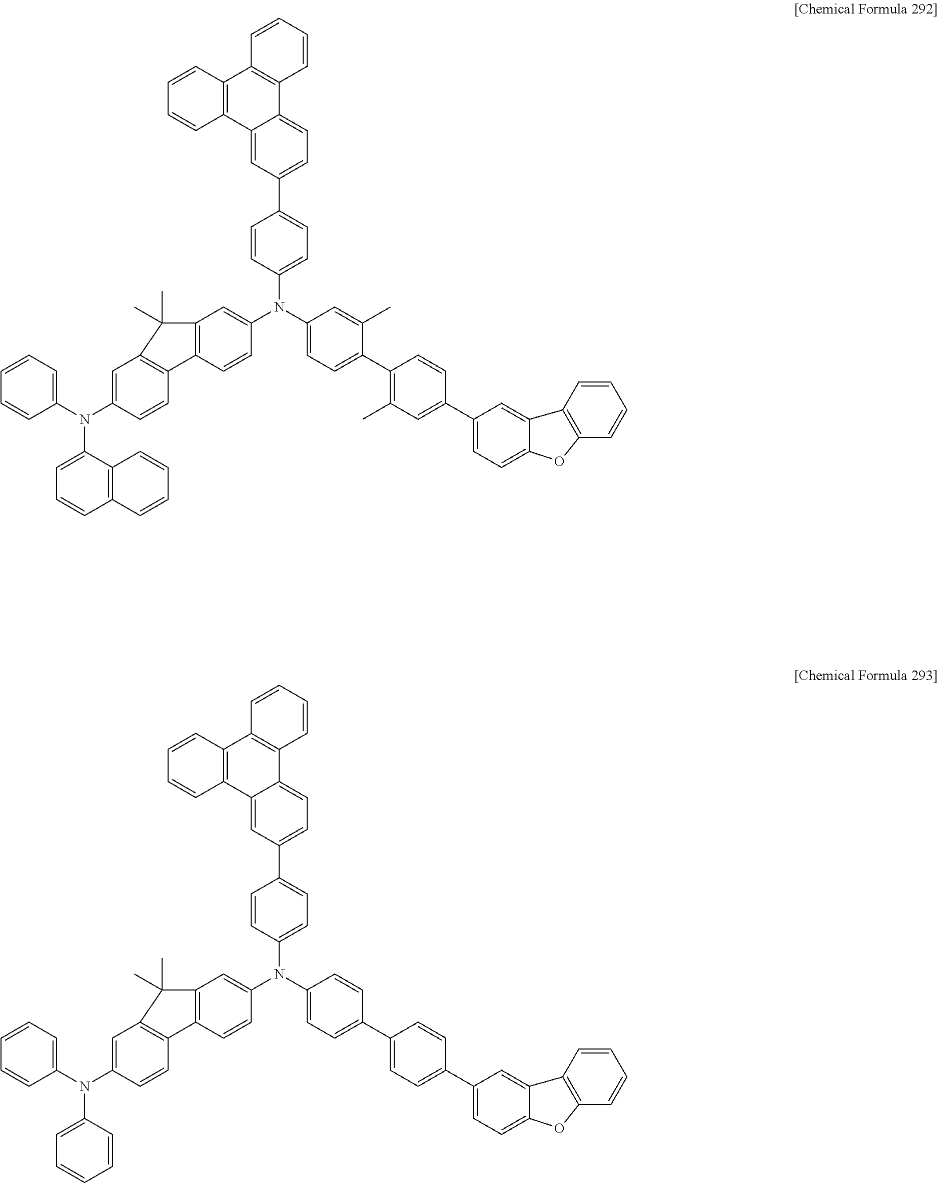 [SMILES: Cc1cc(-c2ccc3oc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5ccc6oc7ccccc7c6c5)cc4)cc3)c3ccc(-c4ccc5c6ccccc6c6ccccc6c5c4)cc3)cc21]

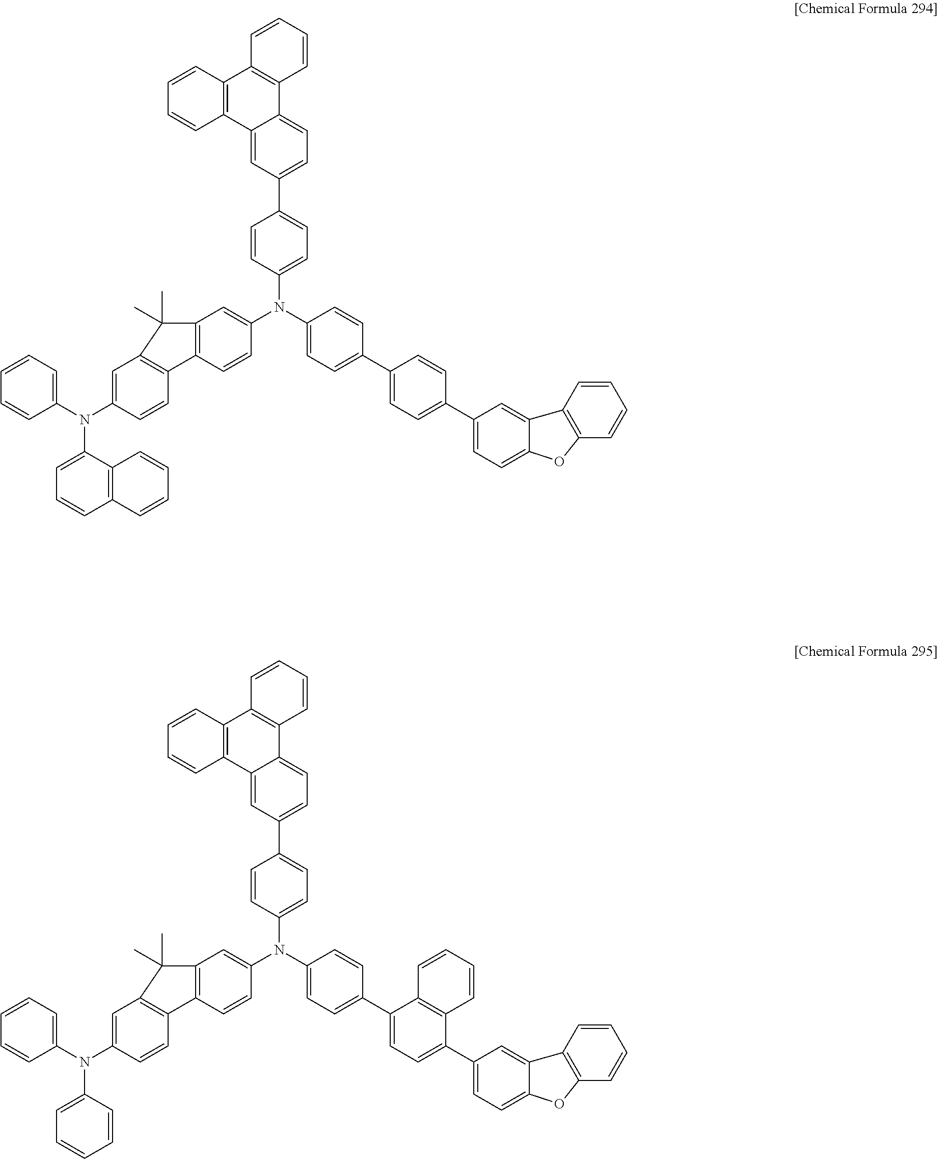 [SMILES: CC1(C)c2cc(N(c3ccc(-c4ccc(-c5ccc6oc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6oc7ccccc7c6c5)c5ccccc45)cc3)cc21]

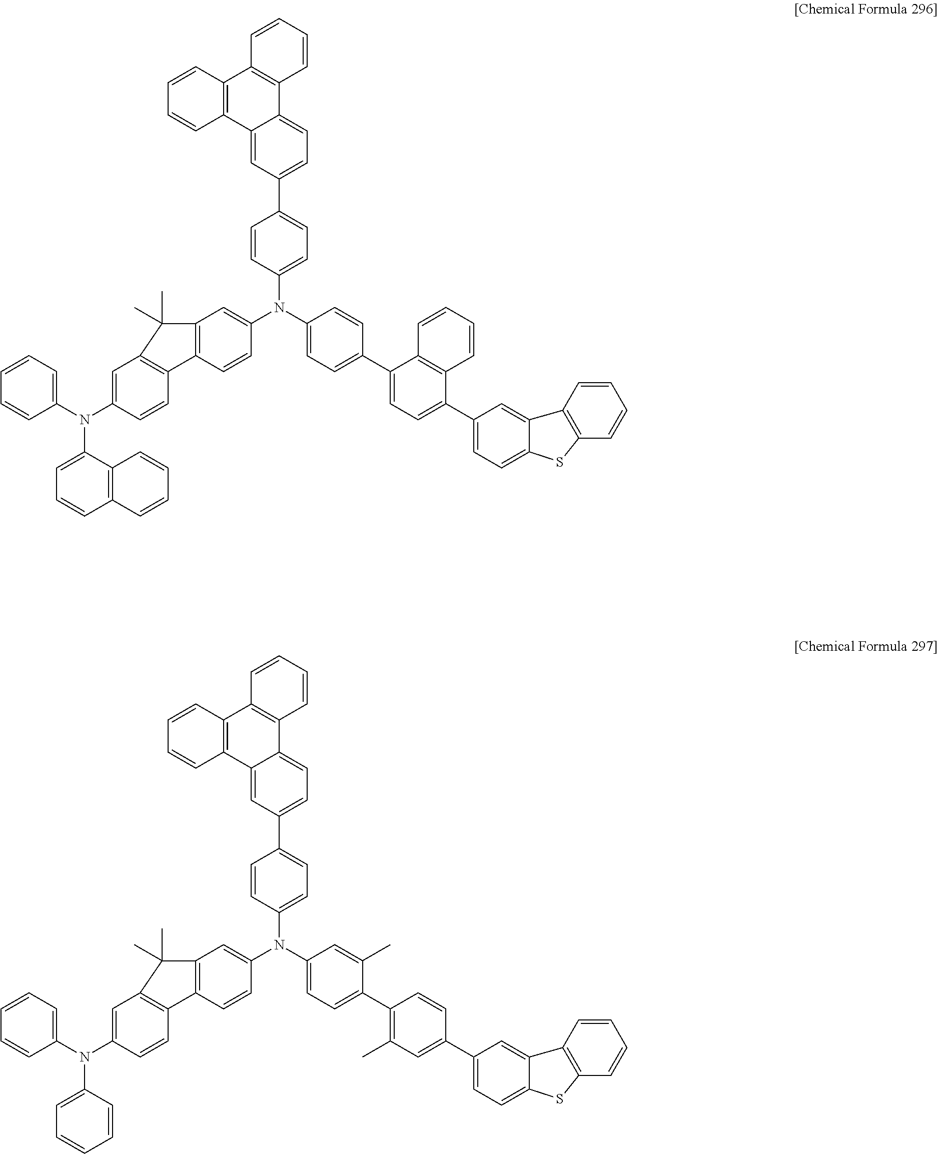 [SMILES: CC1(C)c2cc(N(c3ccc(-c4ccc5c6ccccc6c6ccccc6c5c4)cc3)c3ccc(-c4ccc(-c5ccc6sc7ccccc7c6c5)c5ccccc45)cc3)ccc2-c2ccc(N(c3ccccc3)c3cccc4ccccc34)cc21 ; Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

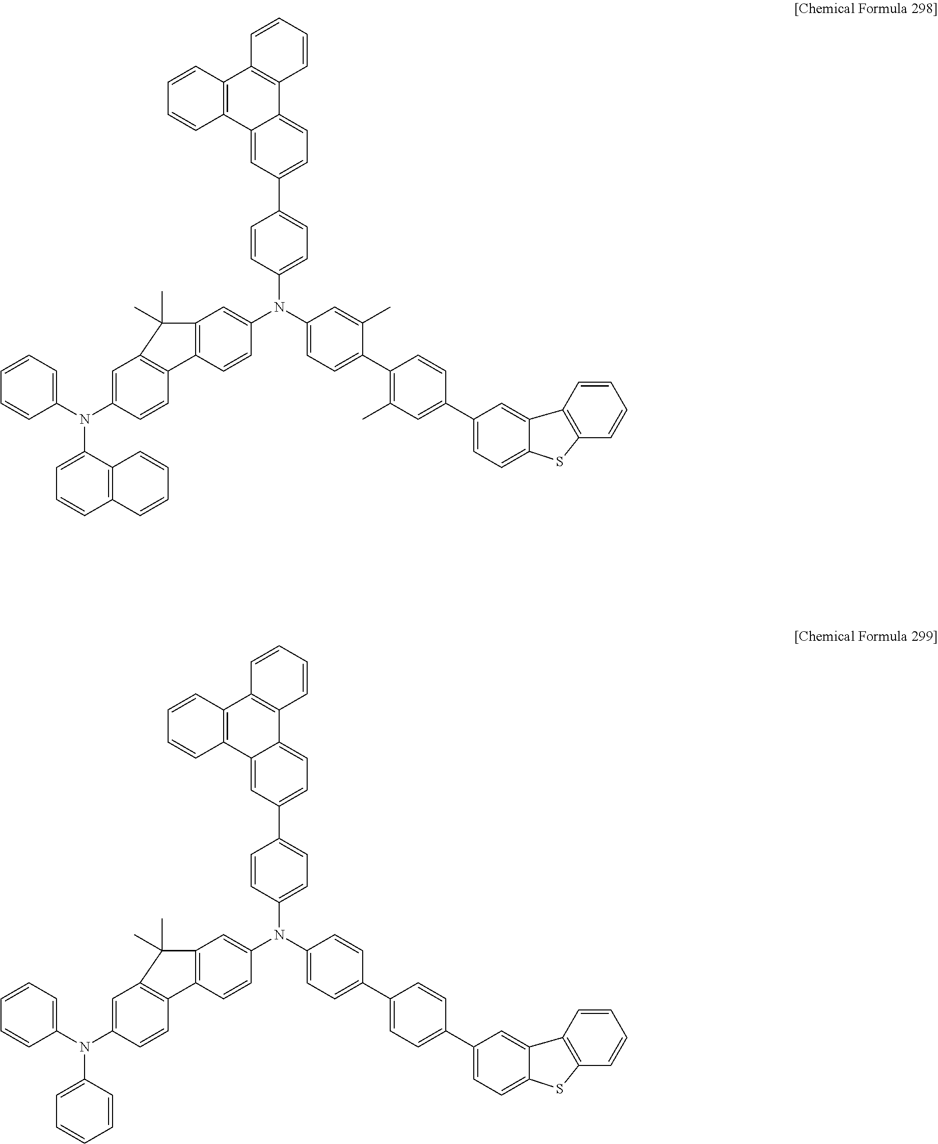 [SMILES: Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5ccc6sc7ccccc7c6c5)cc4)cc3)c3ccc(-c4ccc5c6ccccc6c6ccccc6c5c4)cc3)cc21]

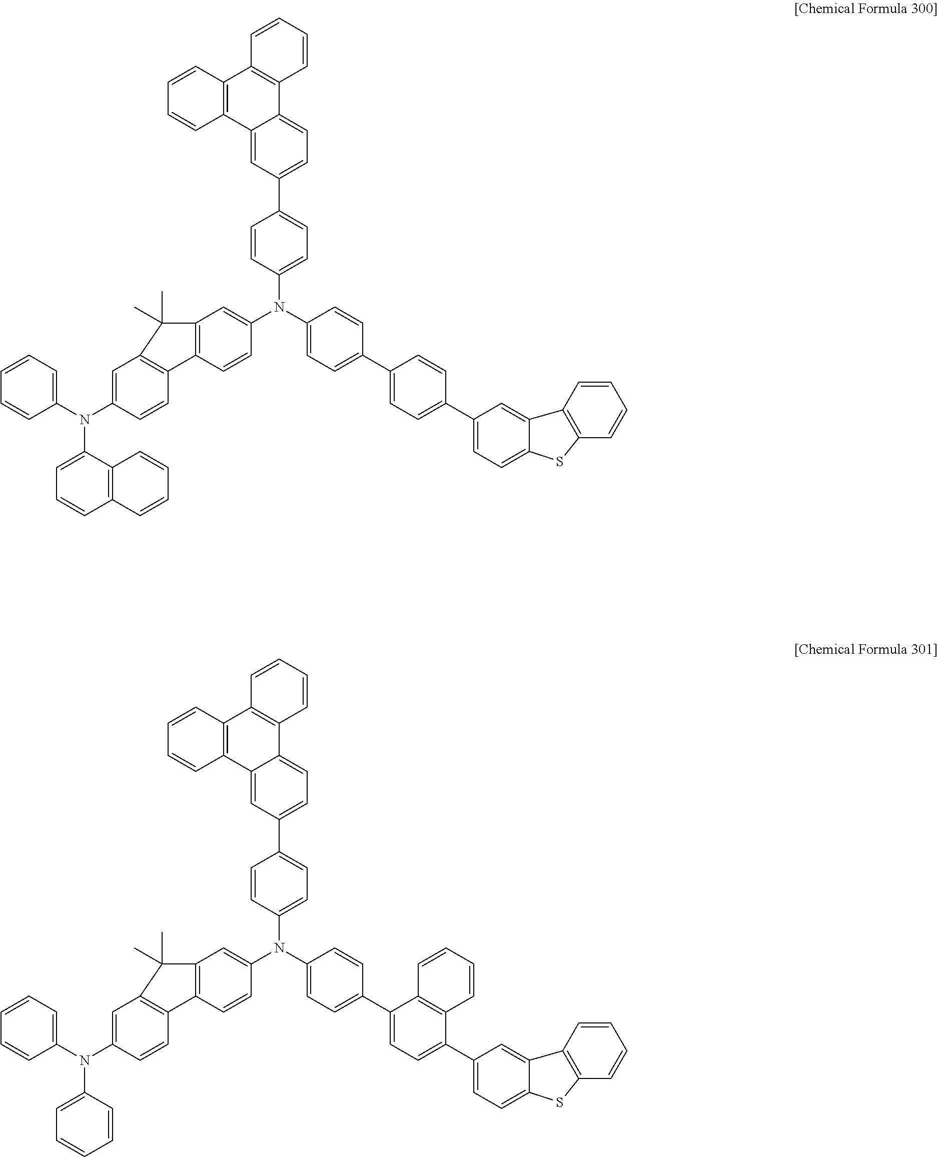 [SMILES: CC1(C)c2cc(N(c3ccc(-c4ccc(-c5ccc6sc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6sc7ccccc7c6c5)c5ccccc45)cc3)cc21]

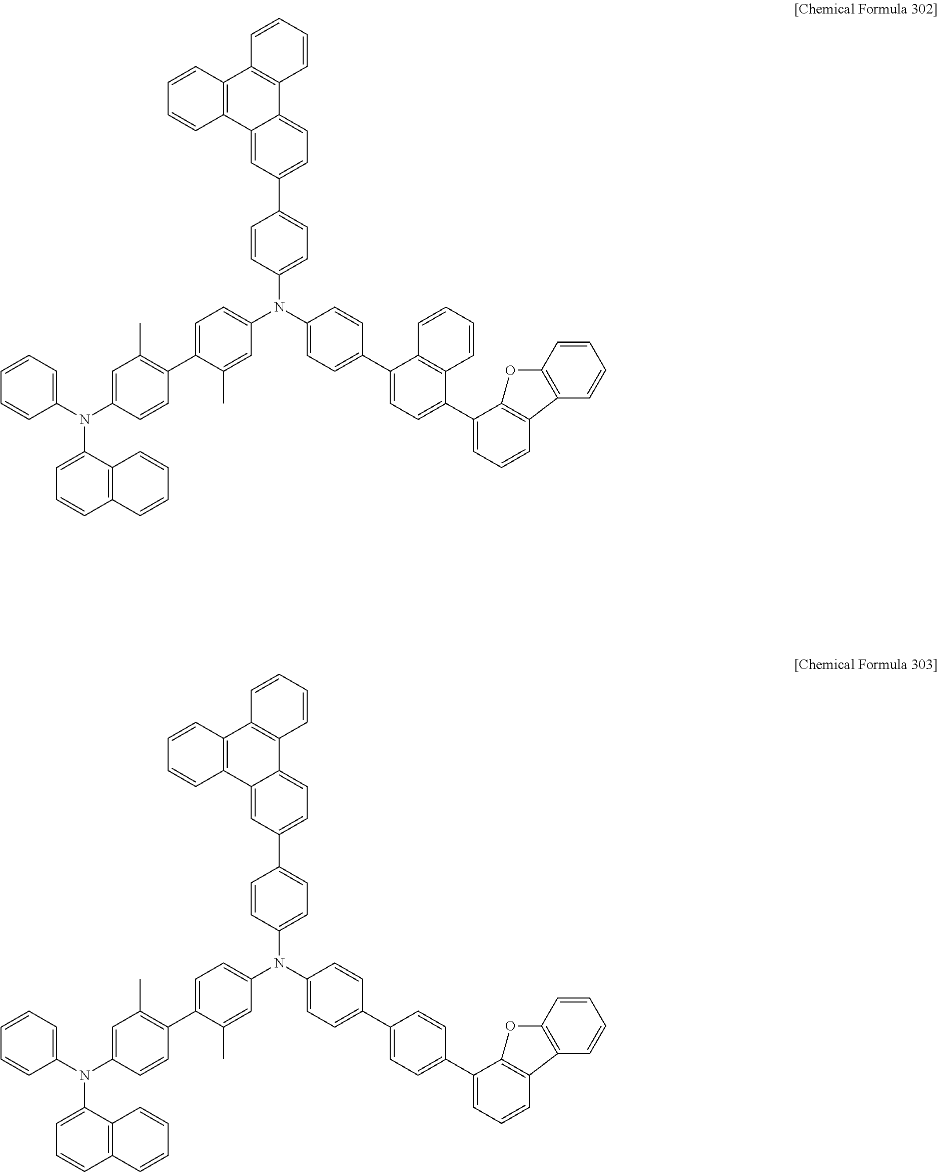 [SMILES: Cc1cc(N(c2ccc(-c3ccc4c5ccccc5c5ccccc5c4c3)cc2)c2ccc(-c3ccc(-c4cccc5c4oc4ccccc45)c4ccccc34)cc2)ccc1-c1ccc(N(c2ccccc2)c2cccc3ccccc23)cc1C ; Cc1cc(N(c2ccc(-c3ccc(-c4cccc5c4oc4ccccc45)cc3)cc2)c2ccc(-c3ccc4c5ccccc5c5ccccc5c4c3)cc2)ccc1-c1ccc(N(c2ccccc2)c2cccc3ccccc23)cc1C]

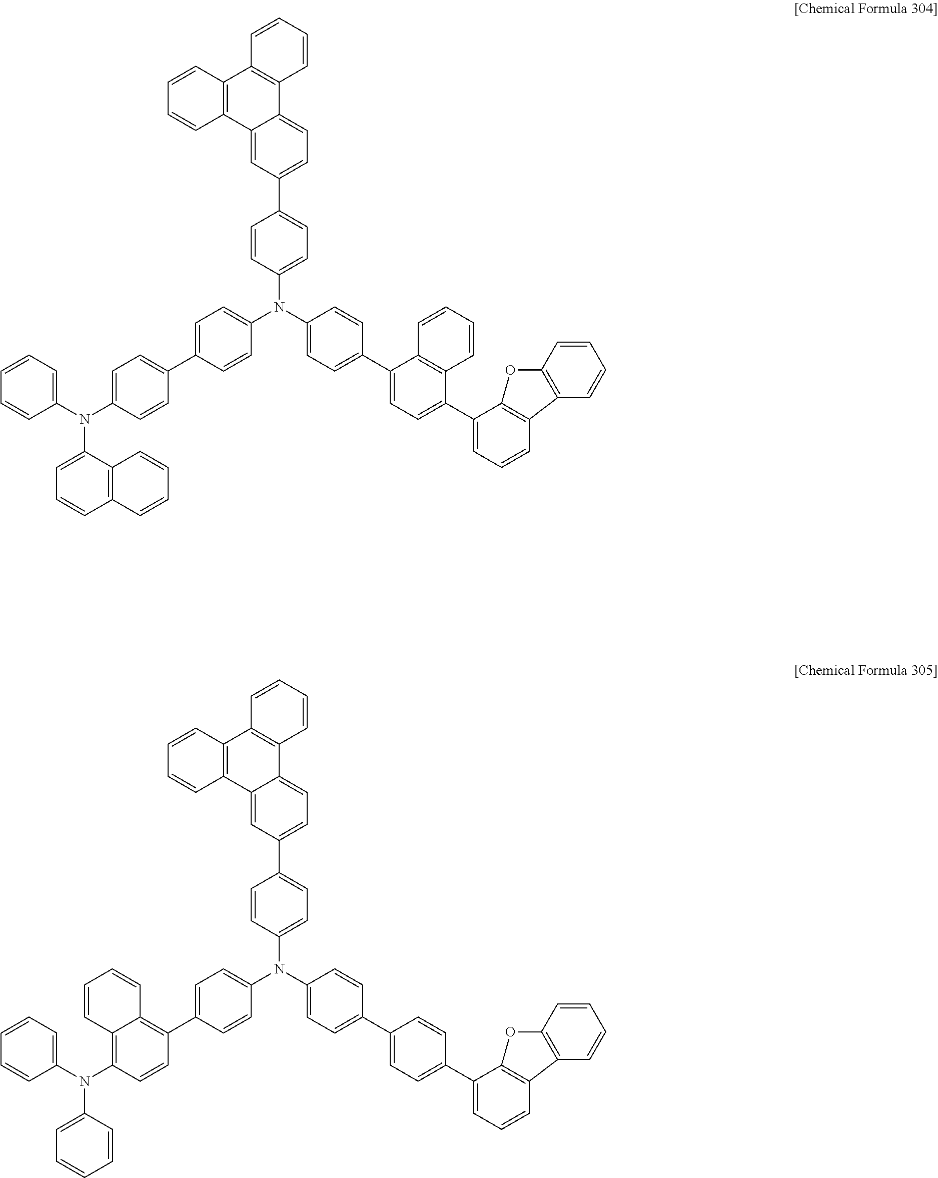 [SMILES: c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1 ; c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)c3ccccc23)cc1]

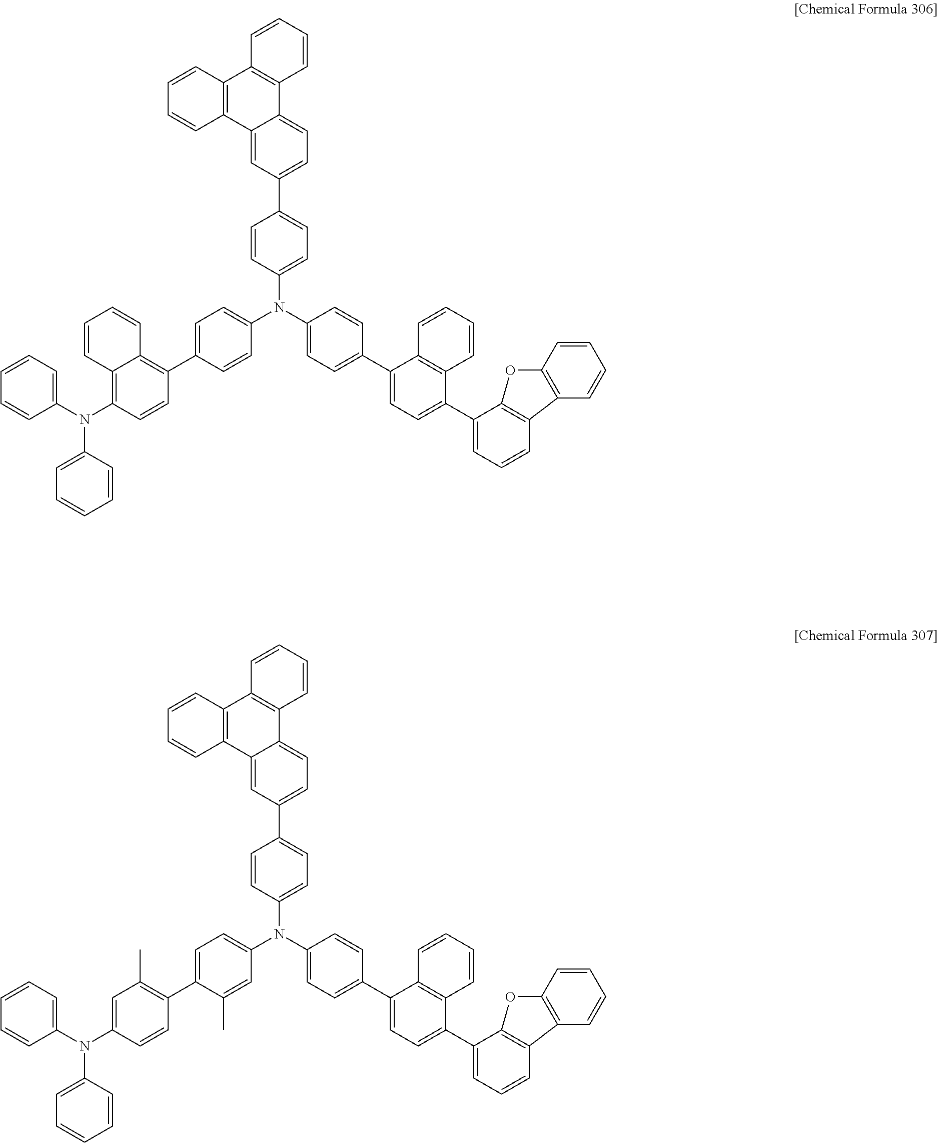 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4oc4ccccc45)c4ccccc34)cc2)cc1C]

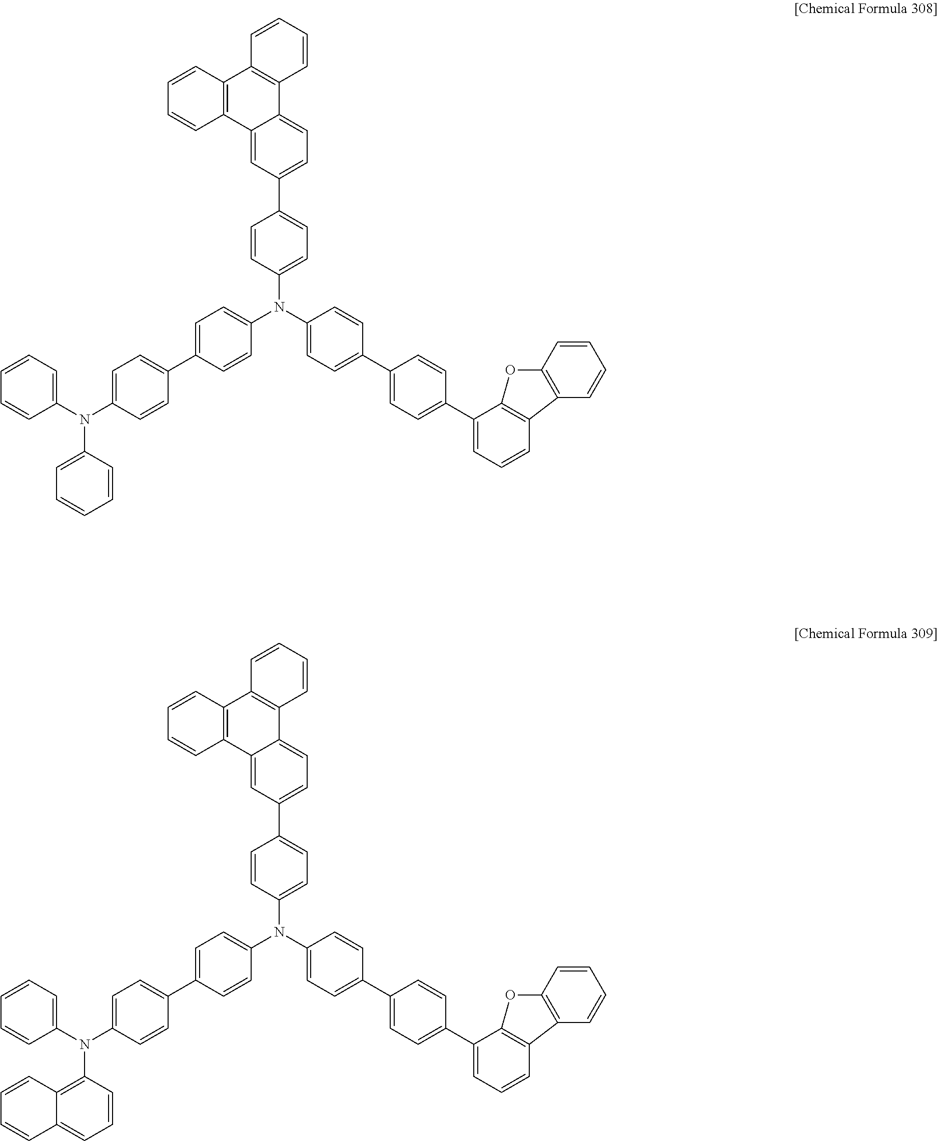 [SMILES: c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)c2cccc3ccccc23)cc1]

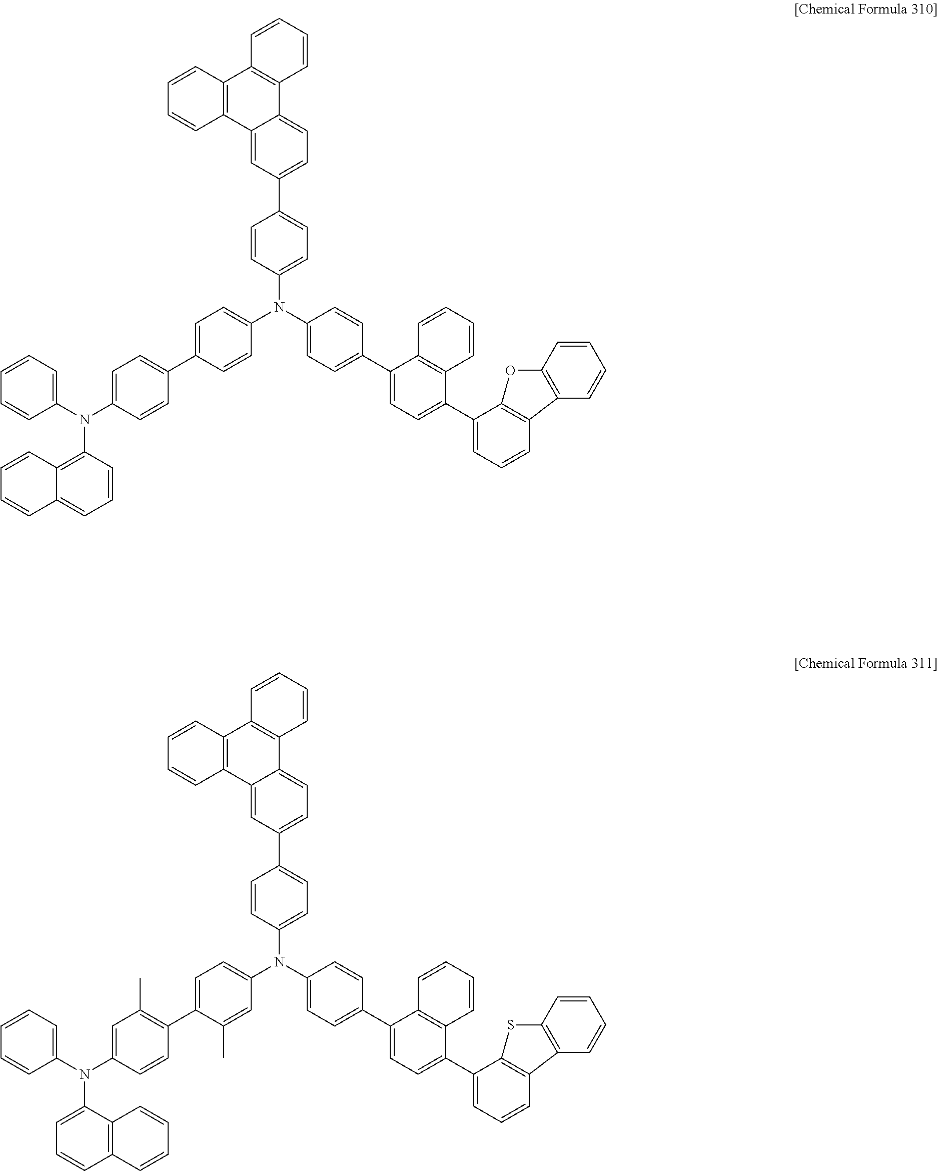 [SMILES: c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1 ; Cc1cc(N(c2ccc(-c3ccc4c5ccccc5c5ccccc5c4c3)cc2)c2ccc(-c3ccc(-c4cccc5c4sc4ccccc45)c4ccccc34)cc2)ccc1-c1ccc(N(c2ccccc2)c2cccc3ccccc23)cc1C]

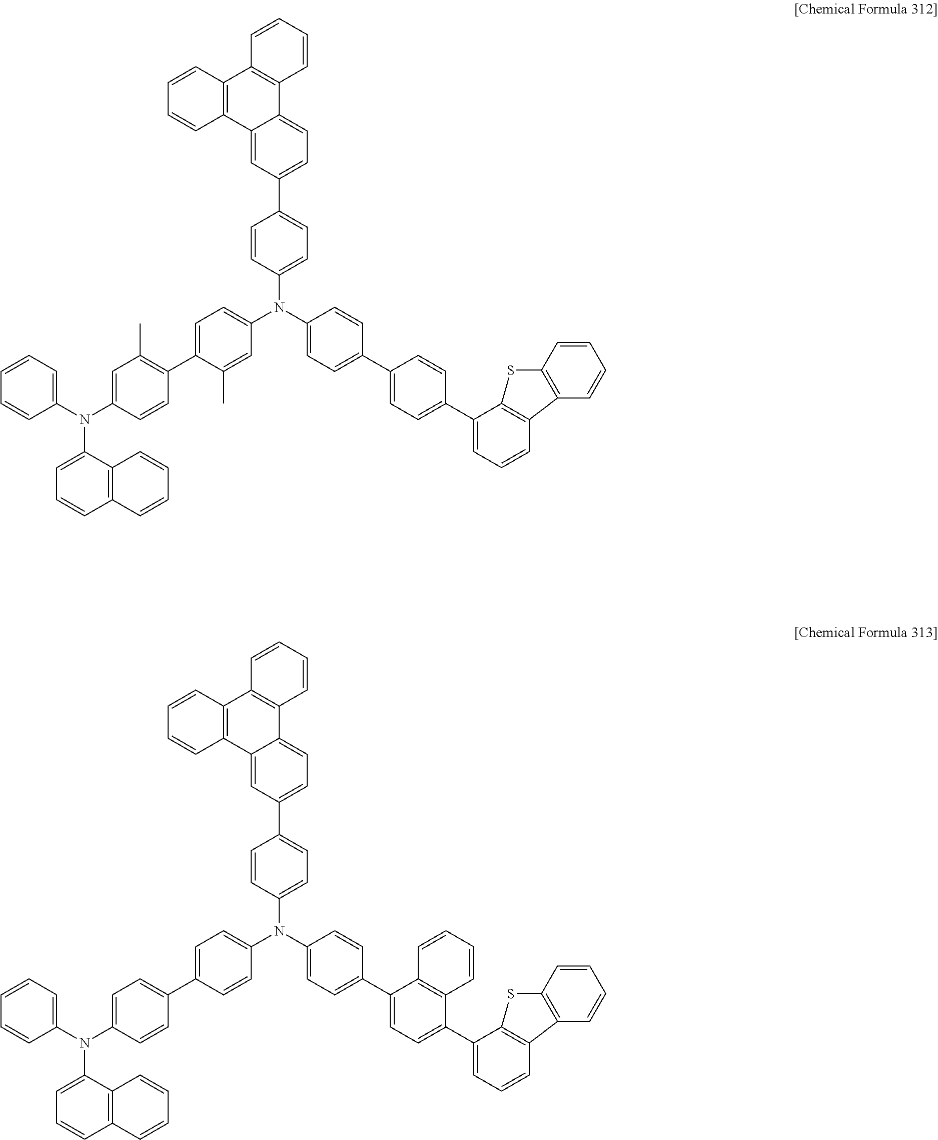 [SMILES: Cc1cc(N(c2ccc(-c3ccc(-c4cccc5c4sc4ccccc45)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1]

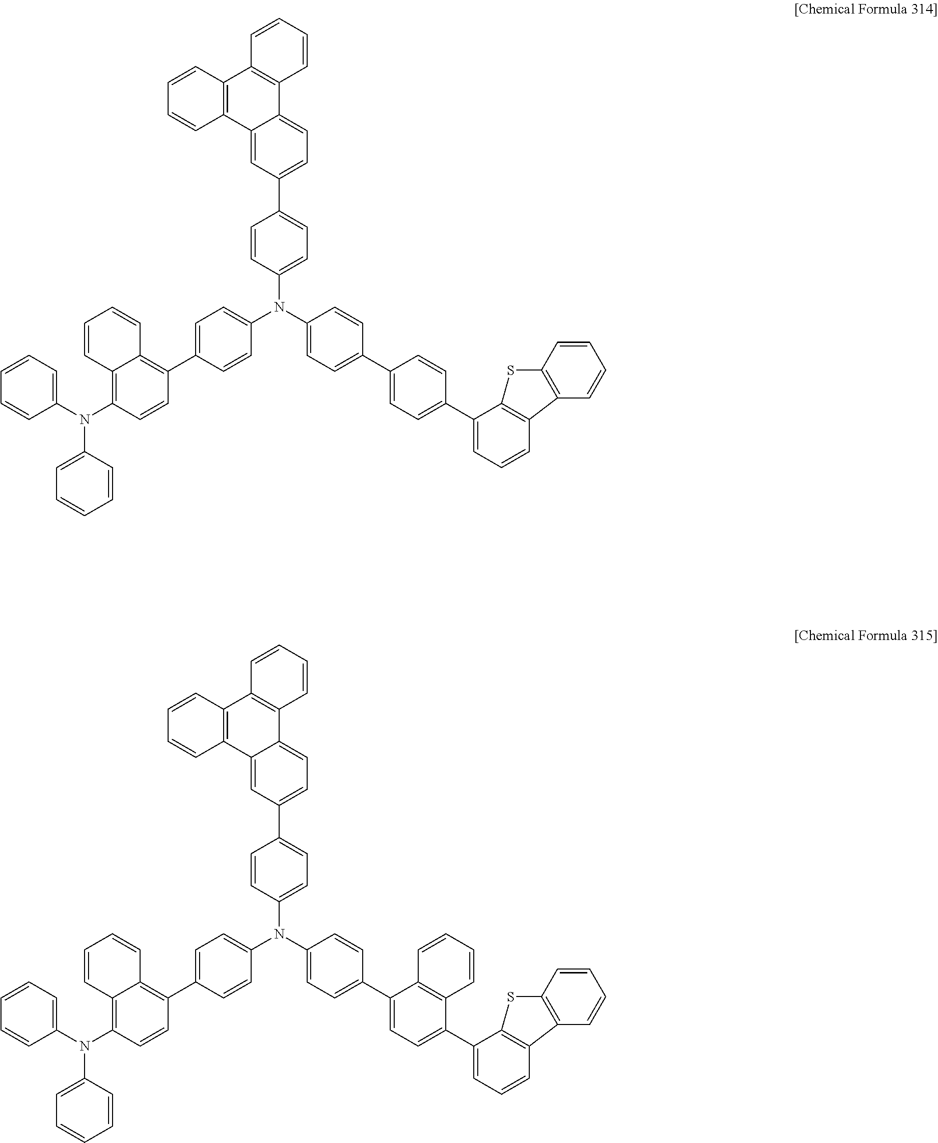 [SMILES: c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)c3ccccc23)cc1]

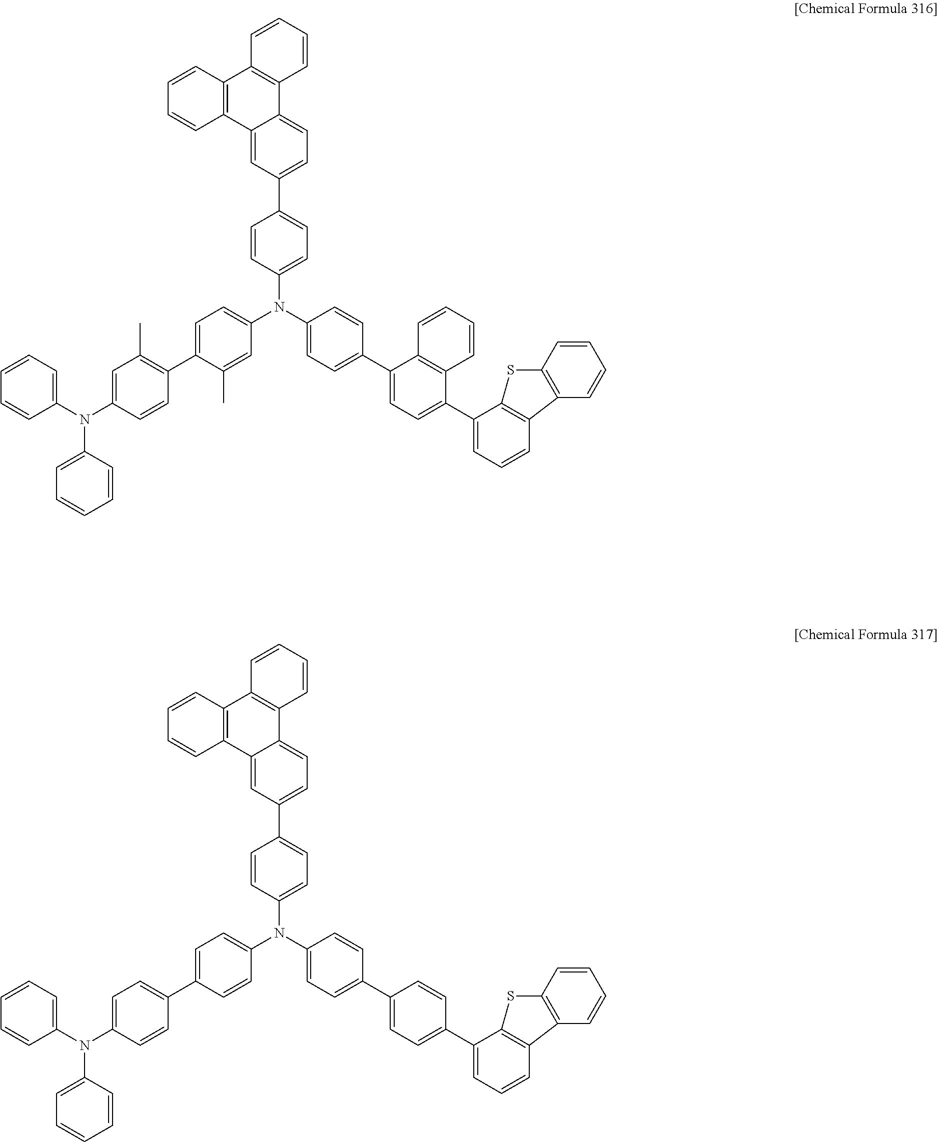 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4sc4ccccc45)c4ccccc34)cc2)cc1C ; c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)cc1]

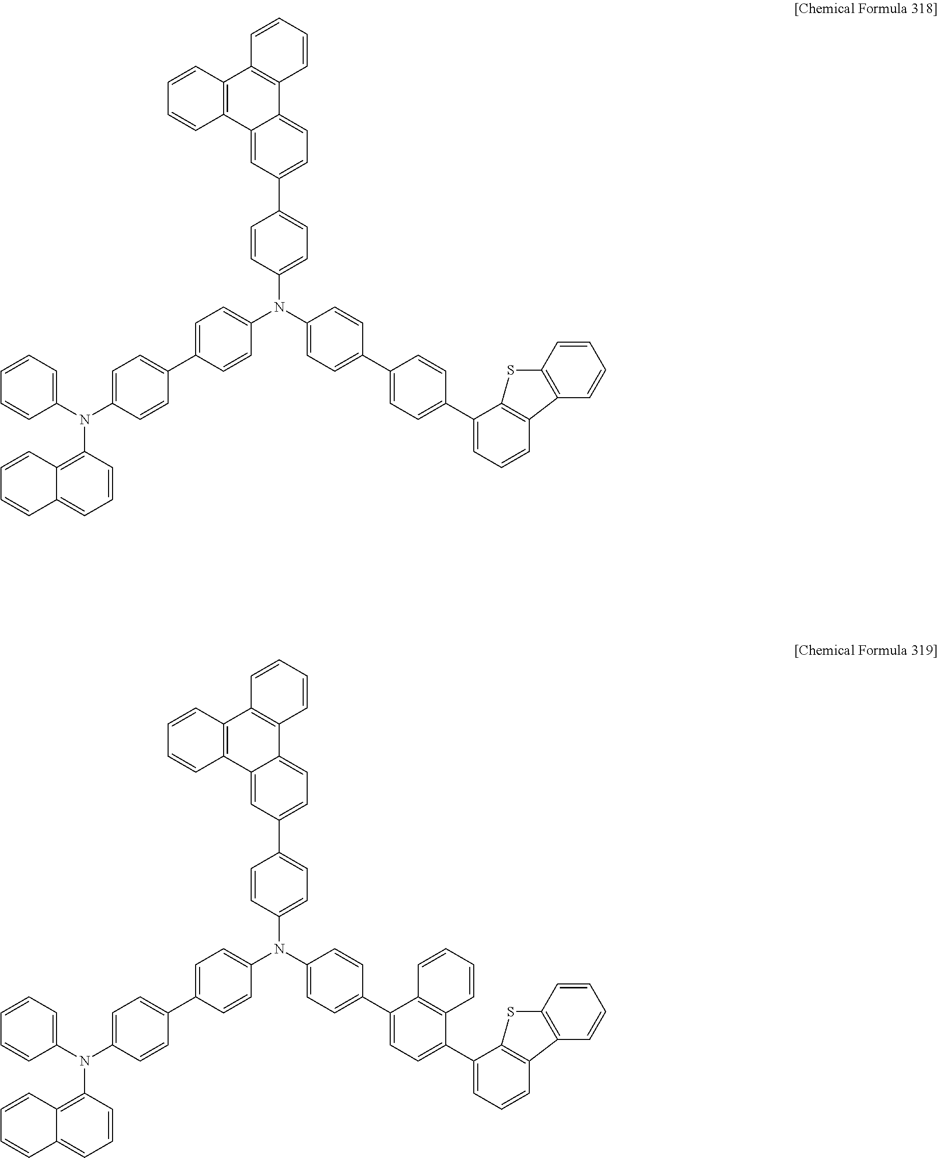 [SMILES: c1ccc(N(c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1]

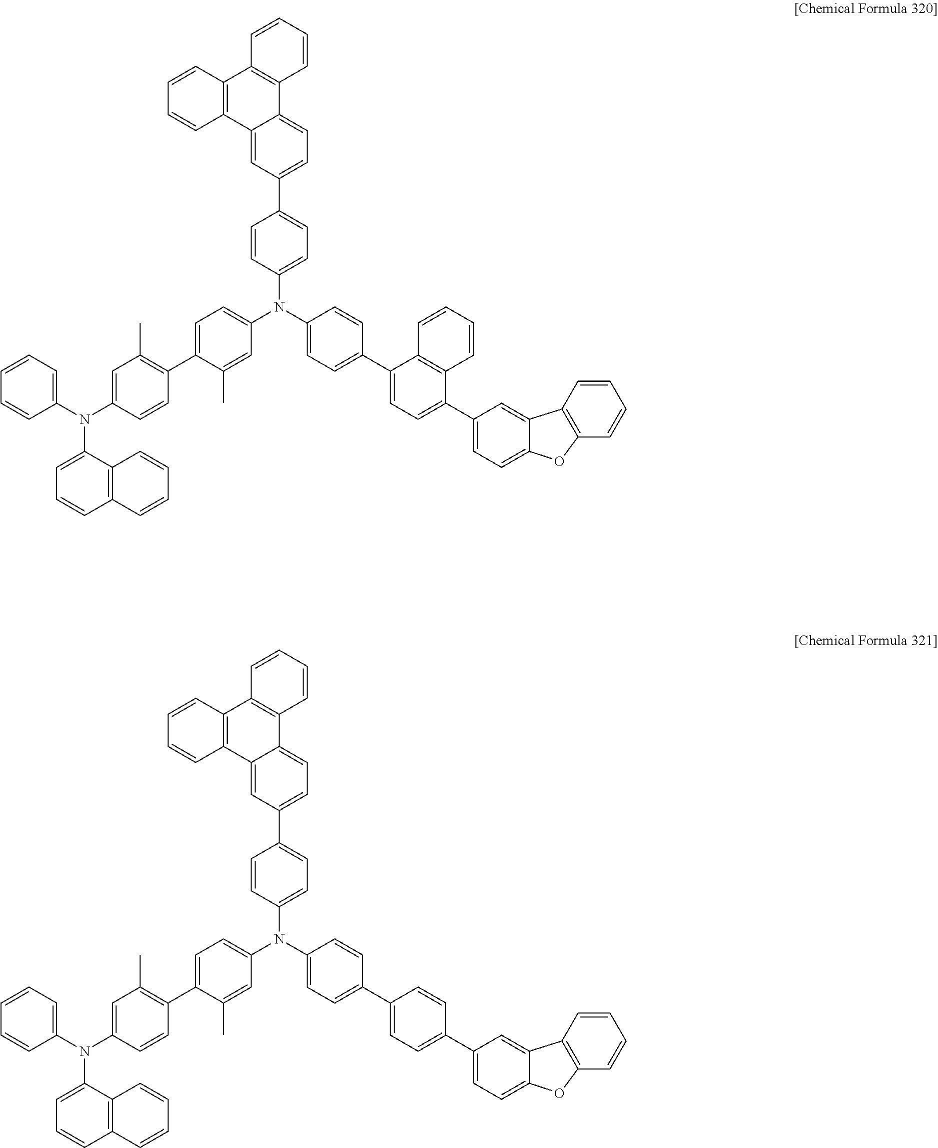 [SMILES: Cc1cc(N(c2ccc(-c3ccc4c5ccccc5c5ccccc5c4c3)cc2)c2ccc(-c3ccc(-c4ccc5oc6ccccc6c5c4)c4ccccc34)cc2)ccc1-c1ccc(N(c2ccccc2)c2cccc3ccccc23)cc1C ; Cc1cc(N(c2ccc(-c3ccc(-c4ccc5oc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C]

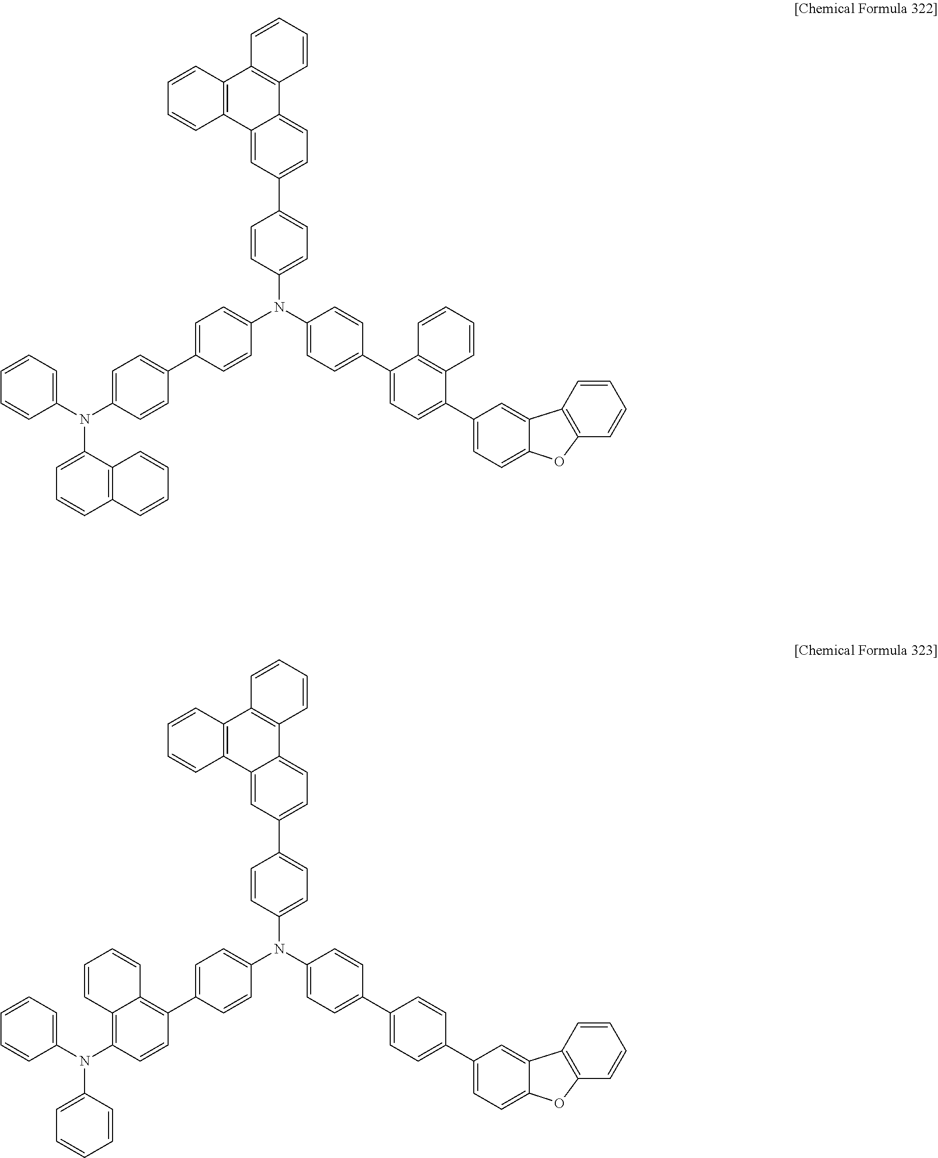 [SMILES: c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1 ; c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)c3ccccc23)cc1]

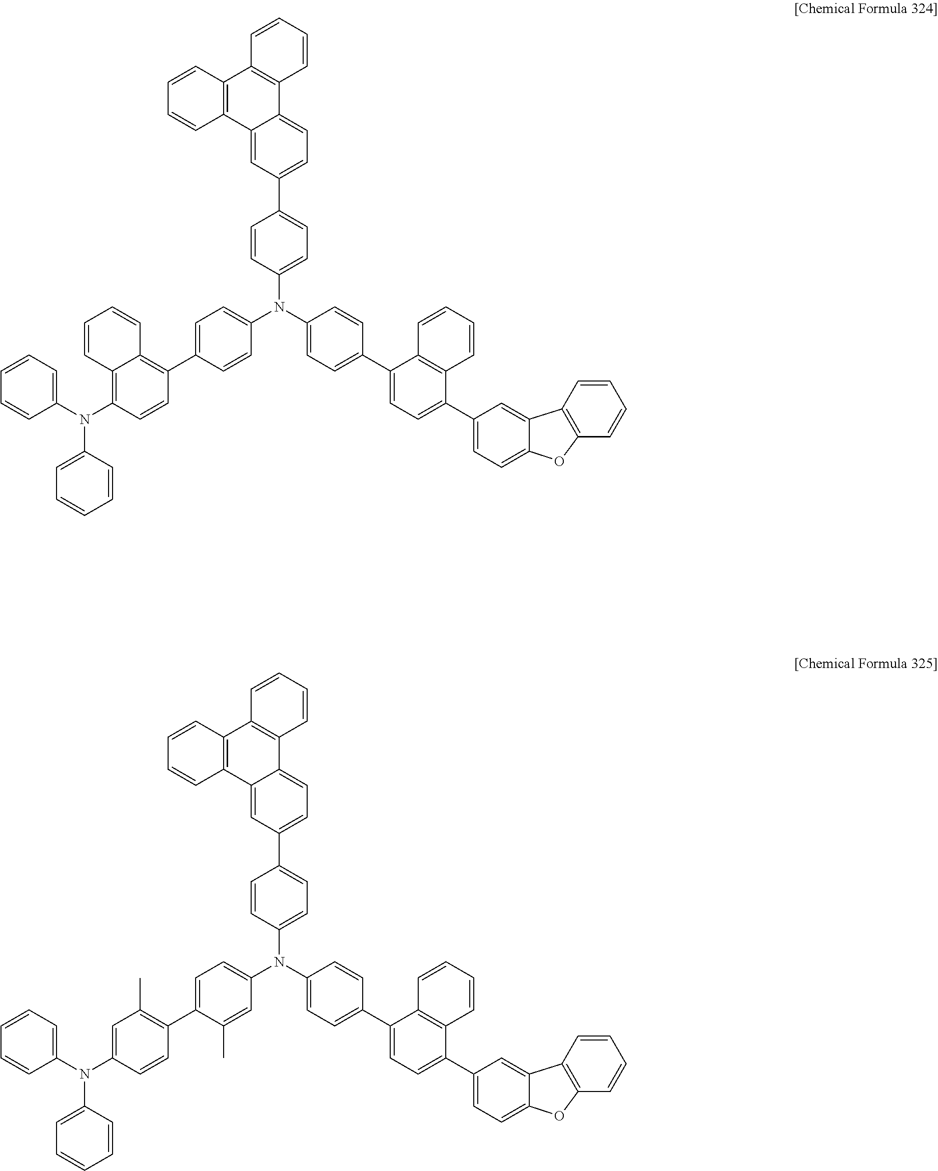 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5oc6ccccc6c5c4)c4ccccc34)cc2)cc1C]

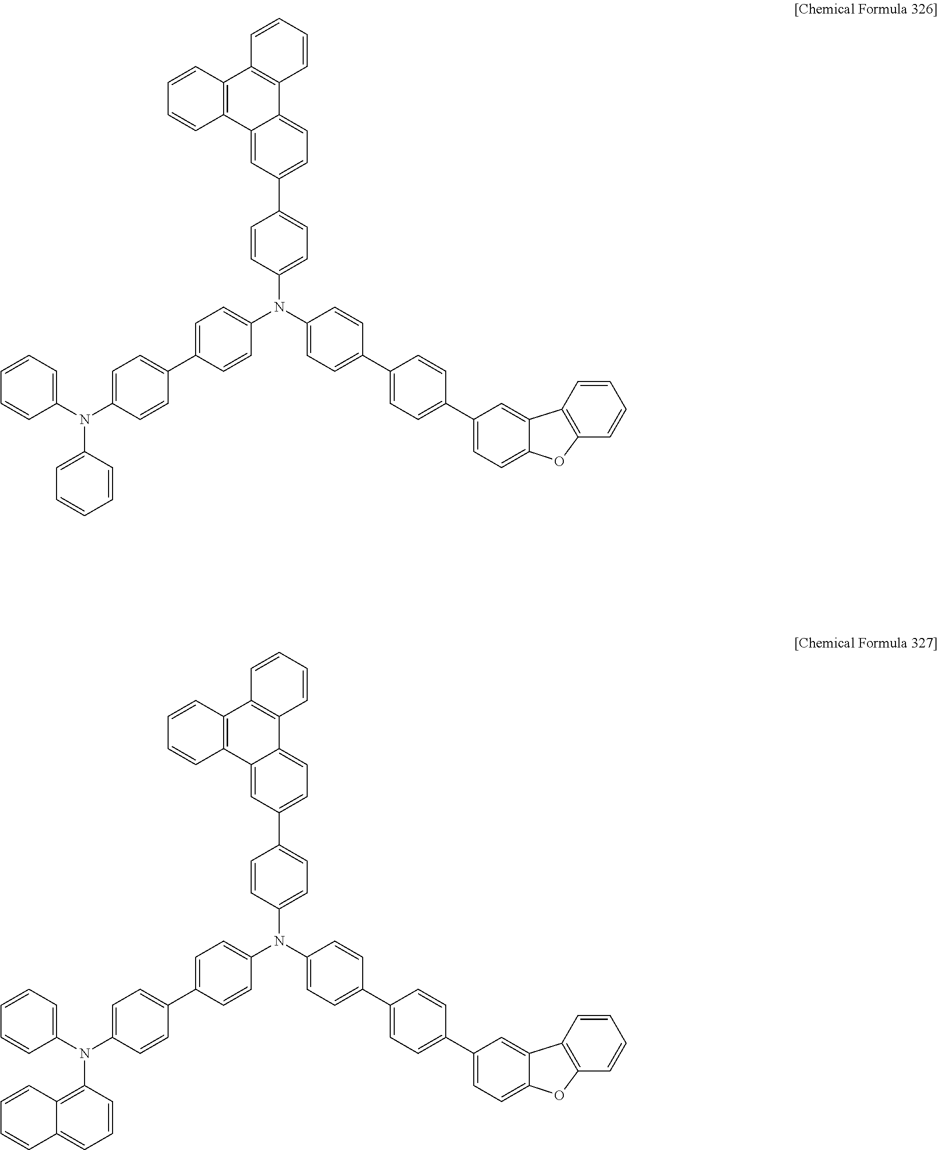 [SMILES: c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)c2cccc3ccccc23)cc1]

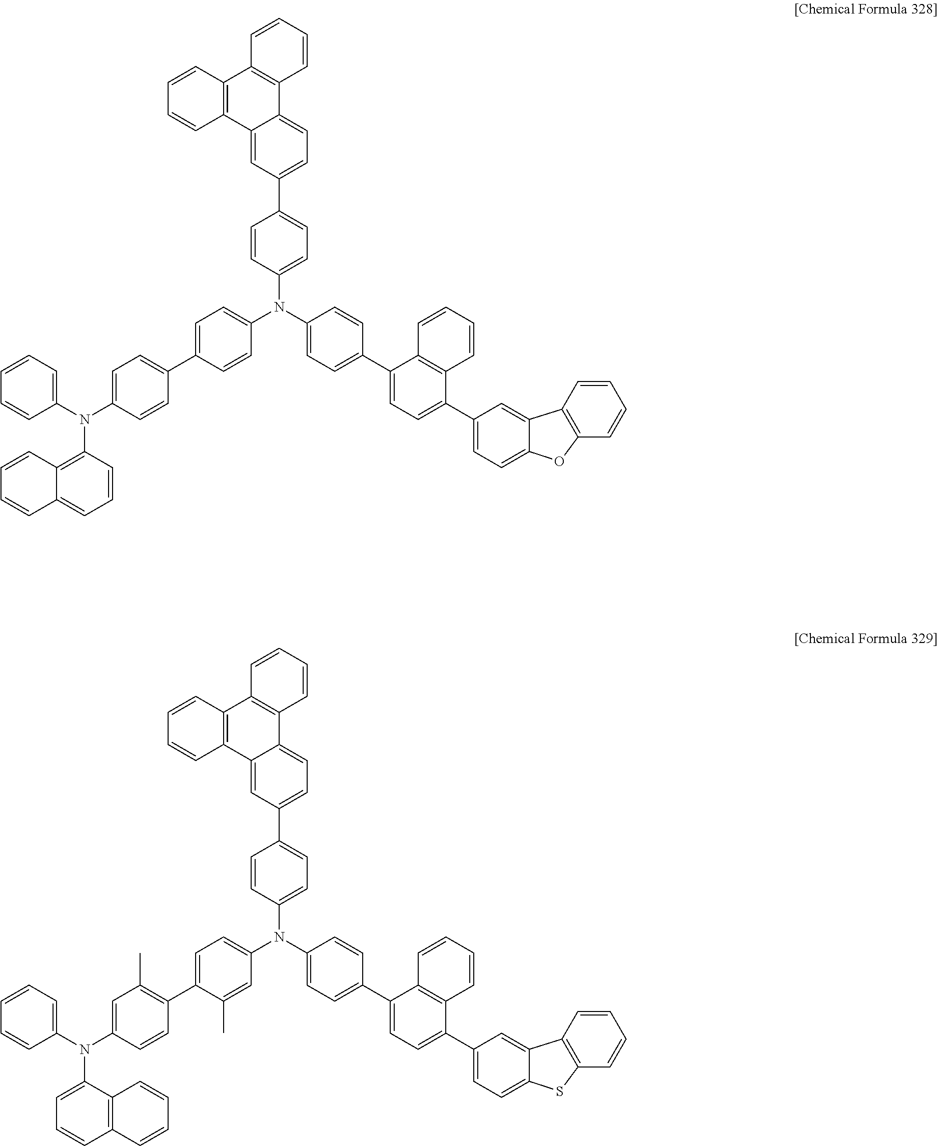 [SMILES: c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1 ; Cc1cc(N(c2ccc(-c3ccc4c5ccccc5c5ccccc5c4c3)cc2)c2ccc(-c3ccc(-c4ccc5sc6ccccc6c5c4)c4ccccc34)cc2)ccc1-c1ccc(N(c2ccccc2)c2cccc3ccccc23)cc1C]

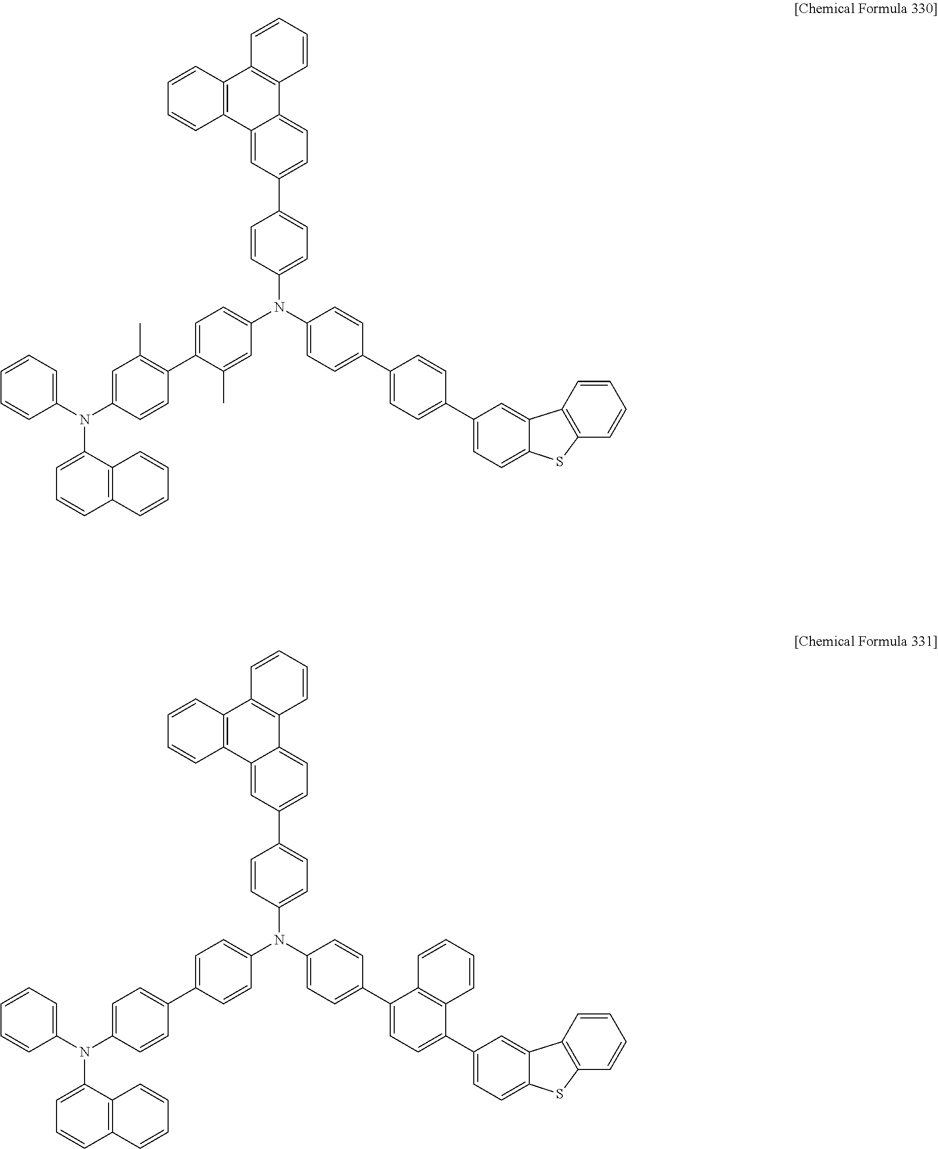 [SMILES: Cc1cc(N(c2ccc(-c3ccc(-c4ccc5sc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1]

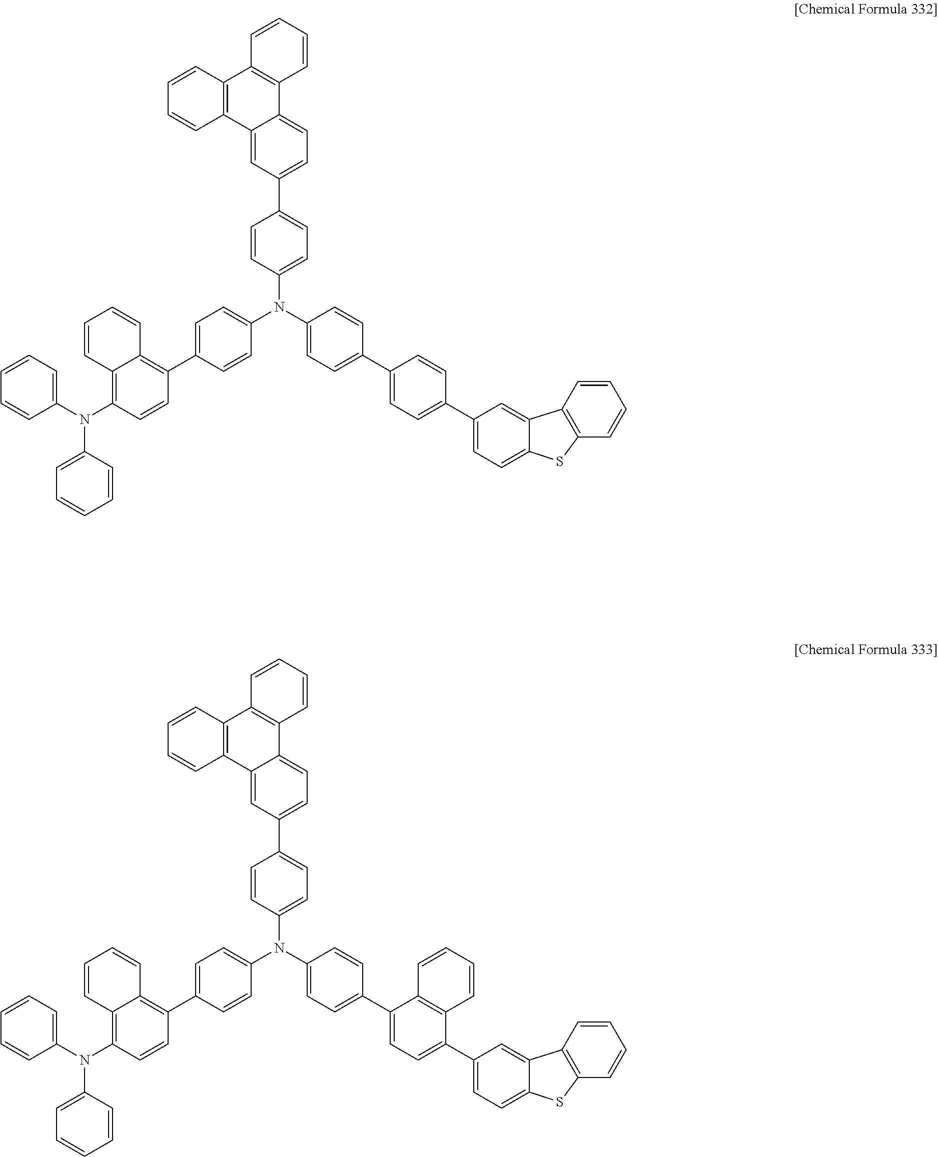 [SMILES: c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)c3ccccc23)cc1]

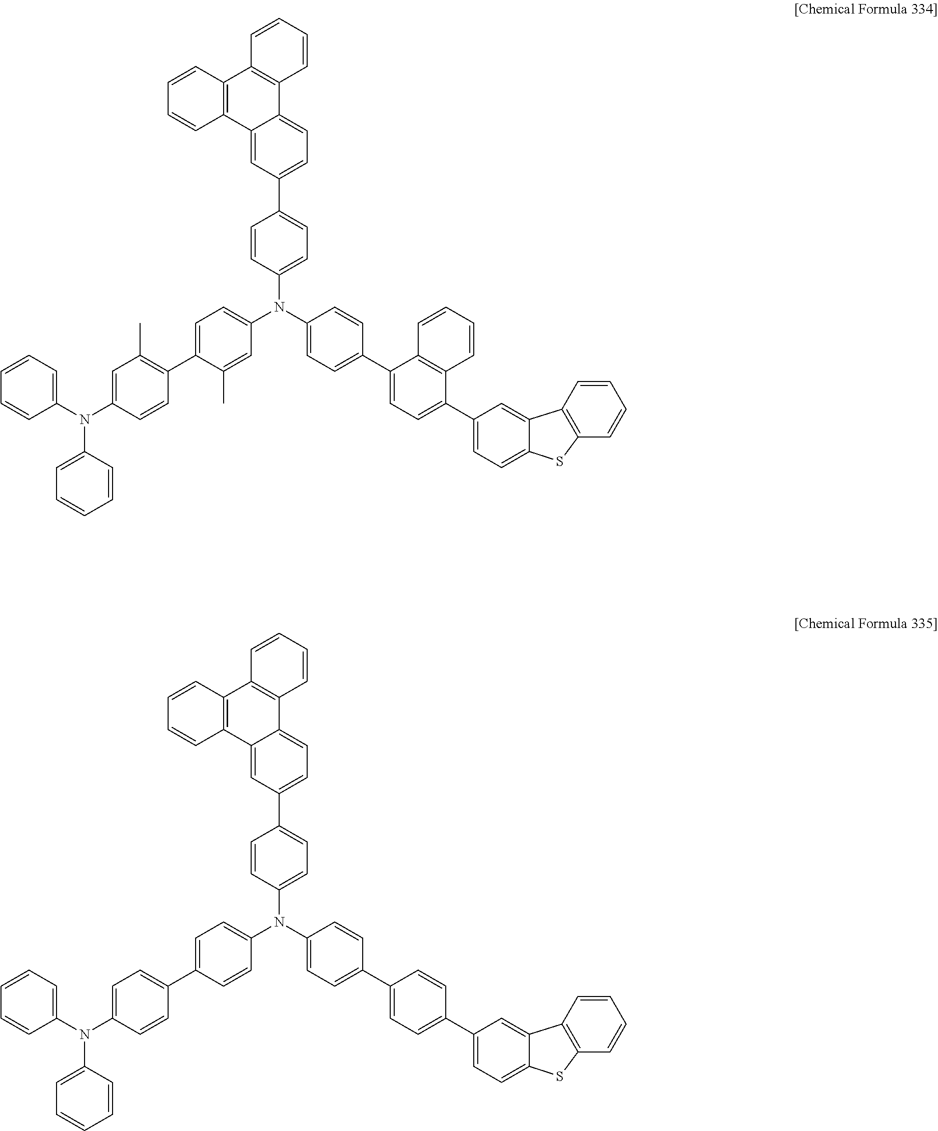 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5sc6ccccc6c5c4)c4ccccc34)cc2)cc1C ; c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)cc1]

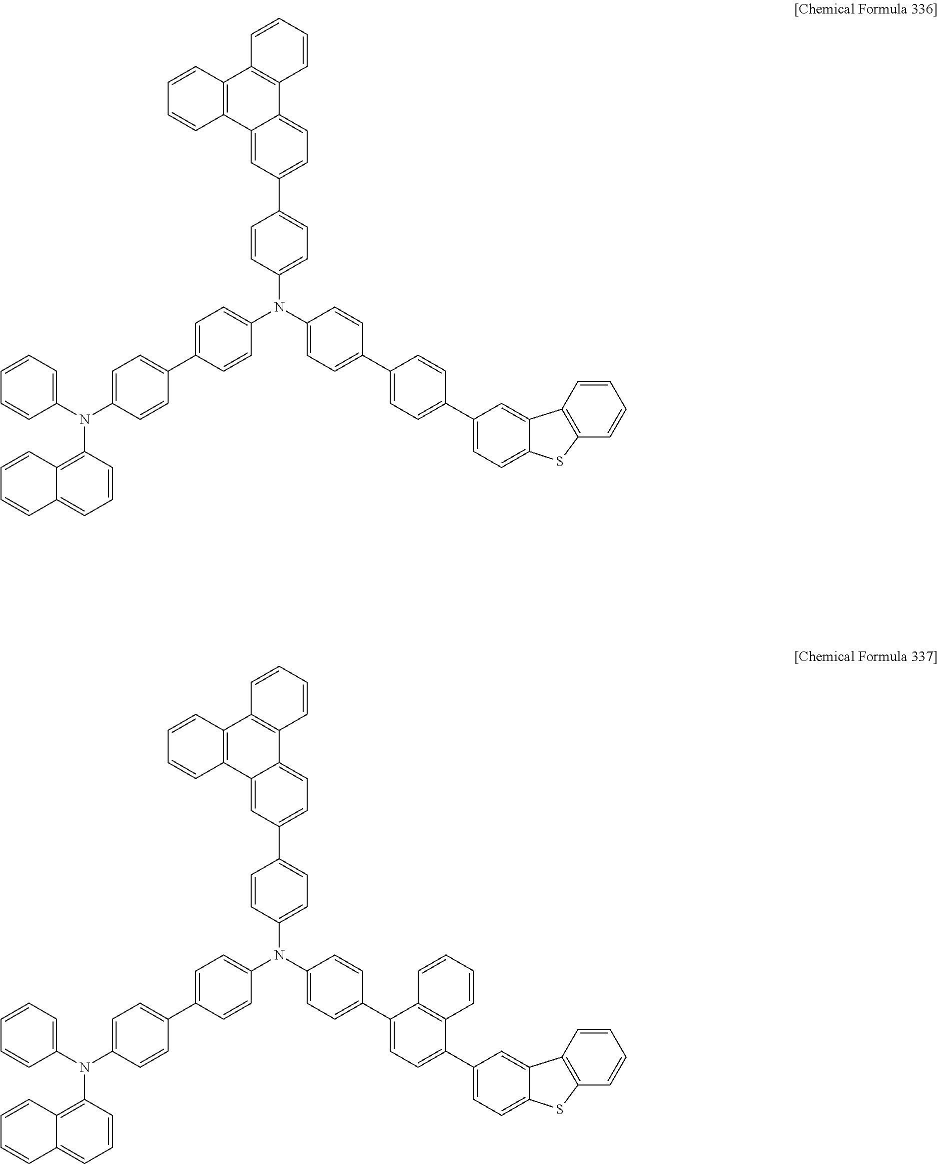 [SMILES: c1ccc(N(c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1]

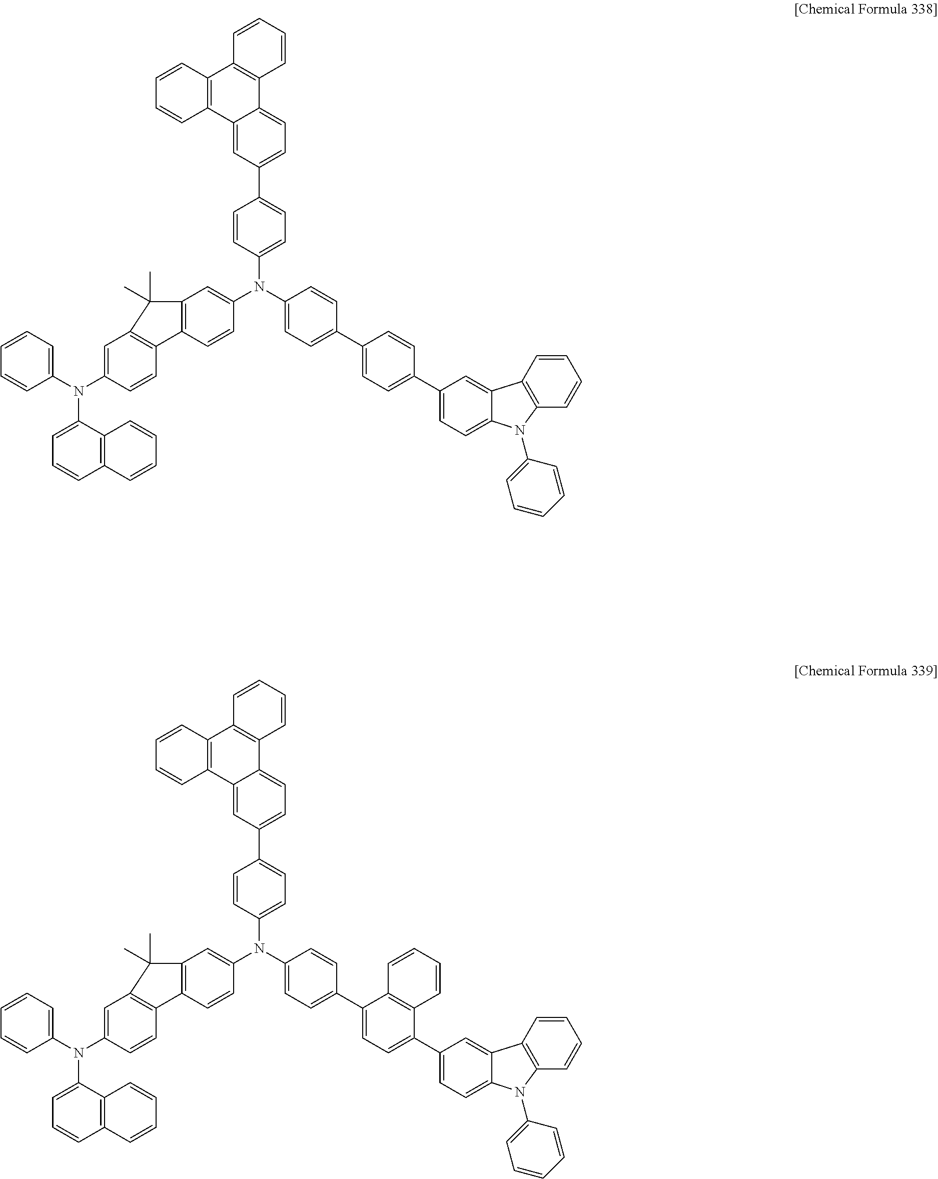 [SMILES: CC1(C)c2cc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)ccc2-c2ccc(N(c3ccccc3)c3cccc4ccccc34)cc21]

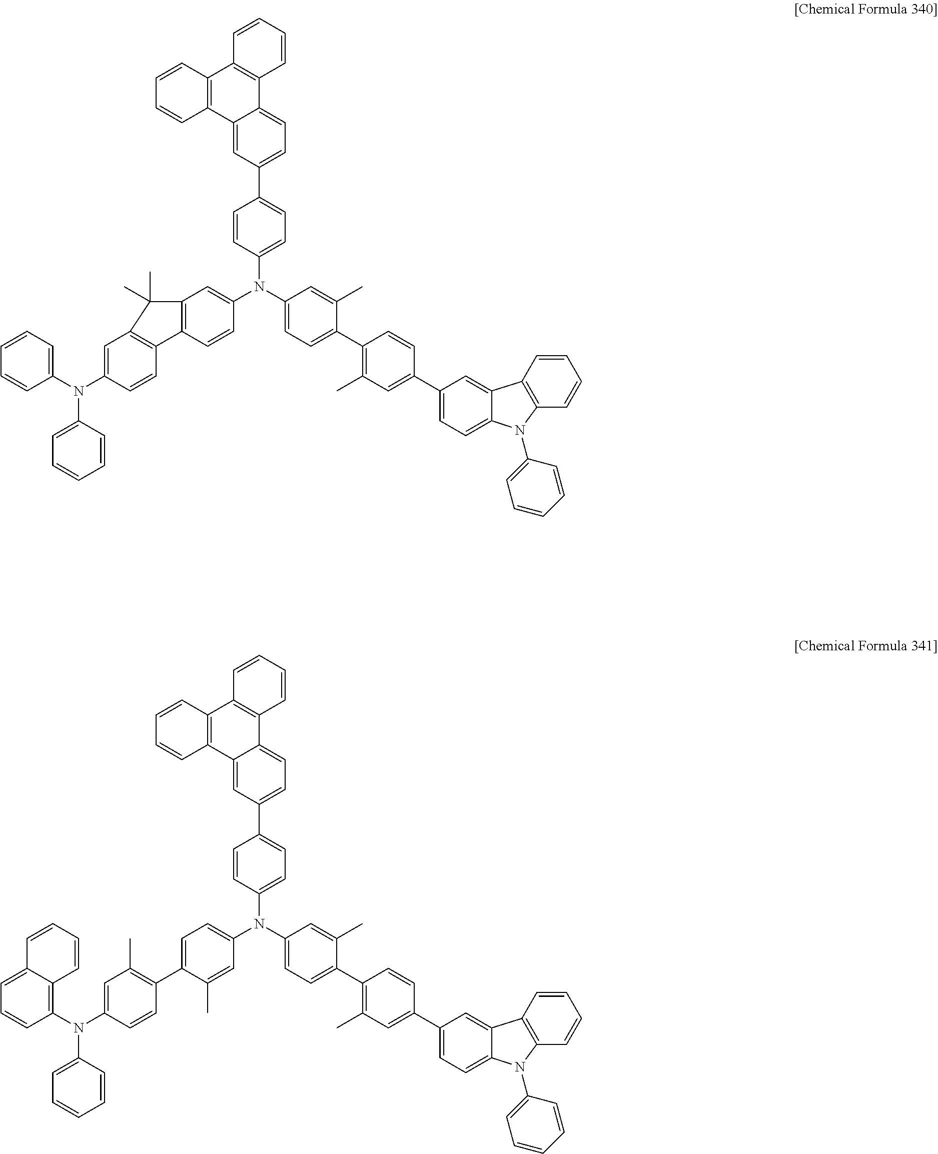 [SMILES: Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc(-c3ccc(N(c4ccccc4)c4cccc5ccccc45)cc3C)c(C)c2)cc1C]

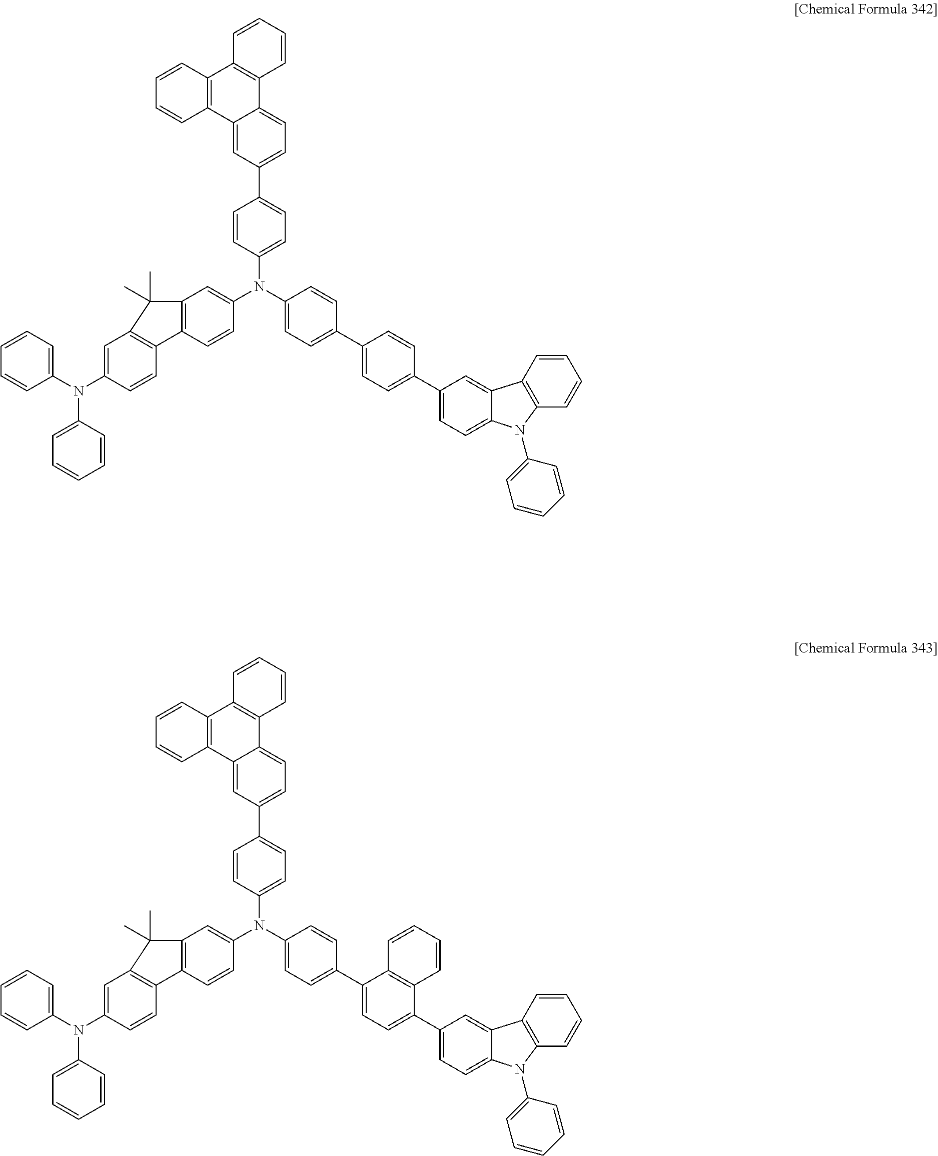 [SMILES: CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)cc21]

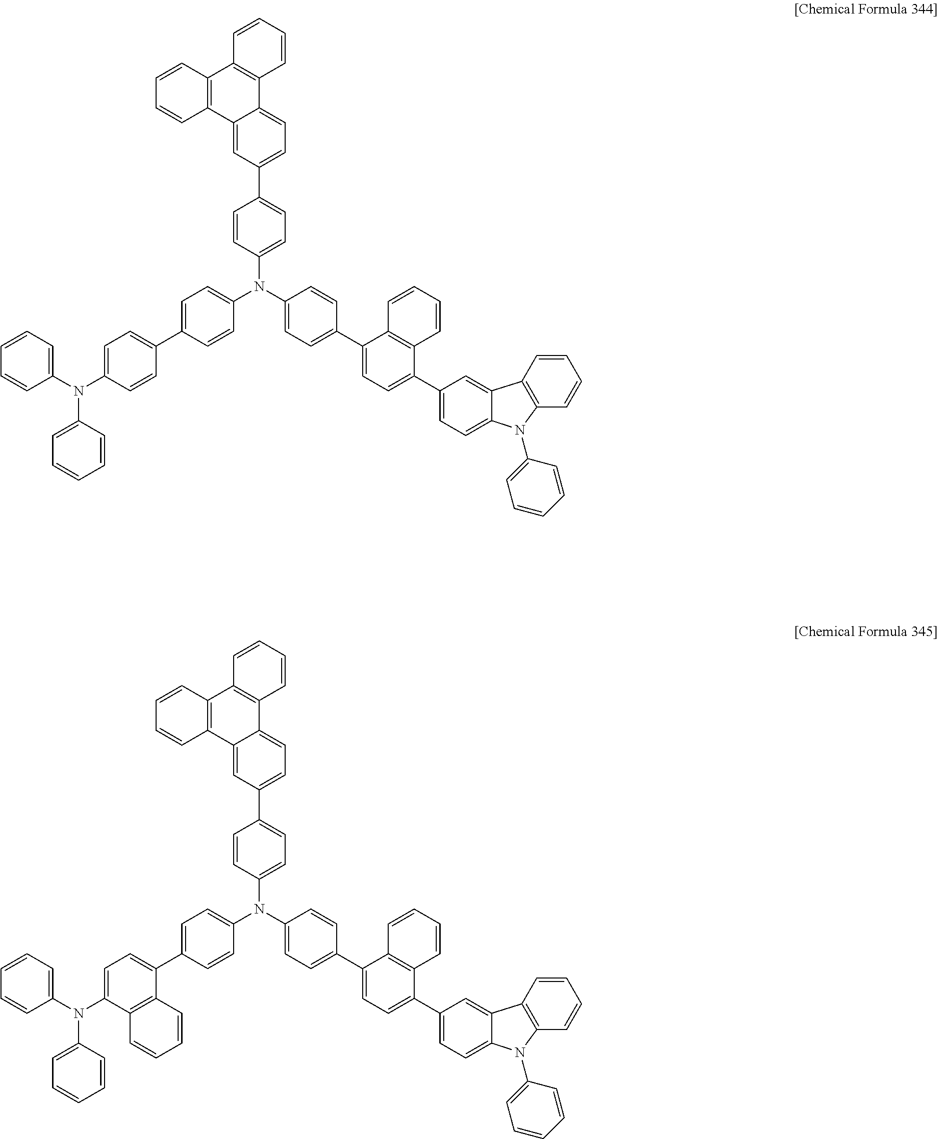 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)cc2)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)c3ccccc23)cc1]

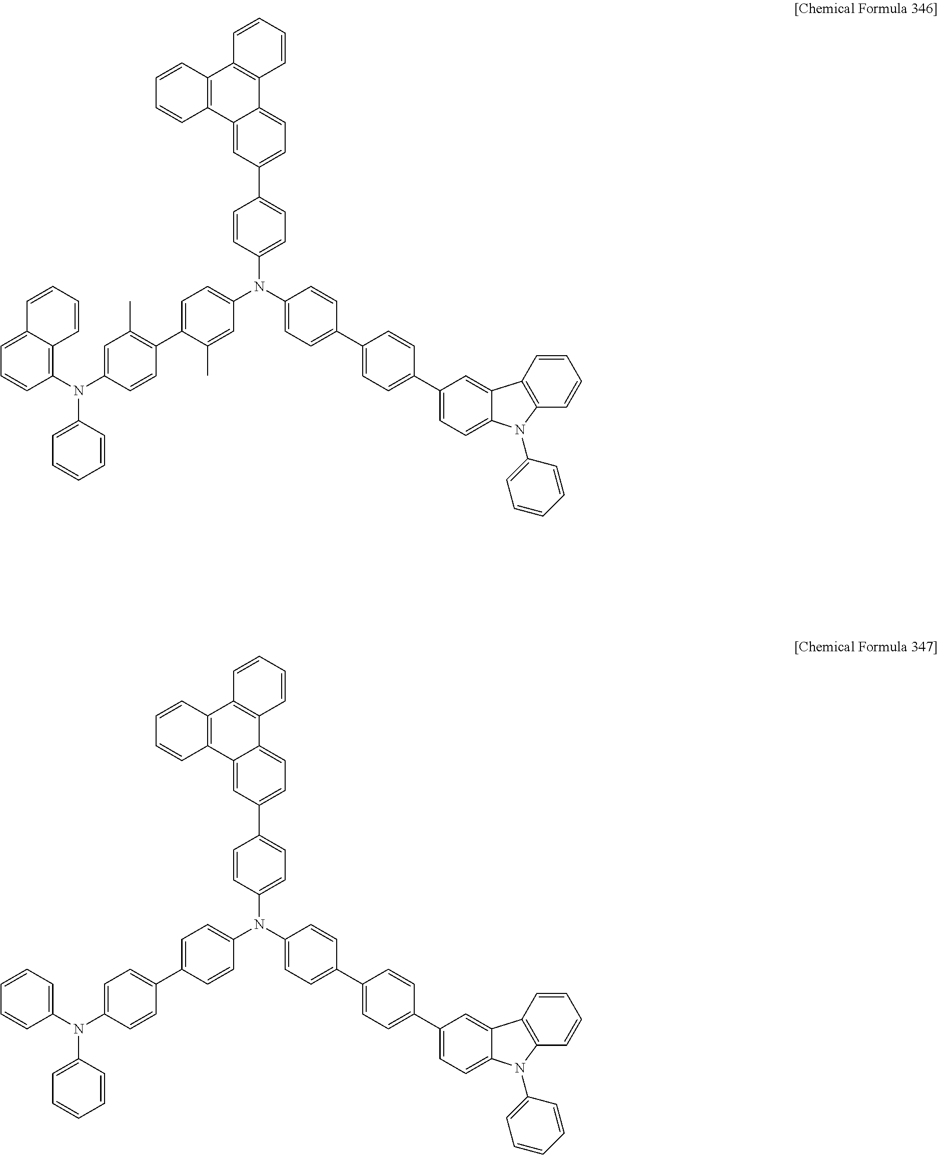 [SMILES: Cc1cc(N(c2ccc(-c3ccc(-c4ccc5c(c4)c4ccccc4n5-c4ccccc4)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)cc1]

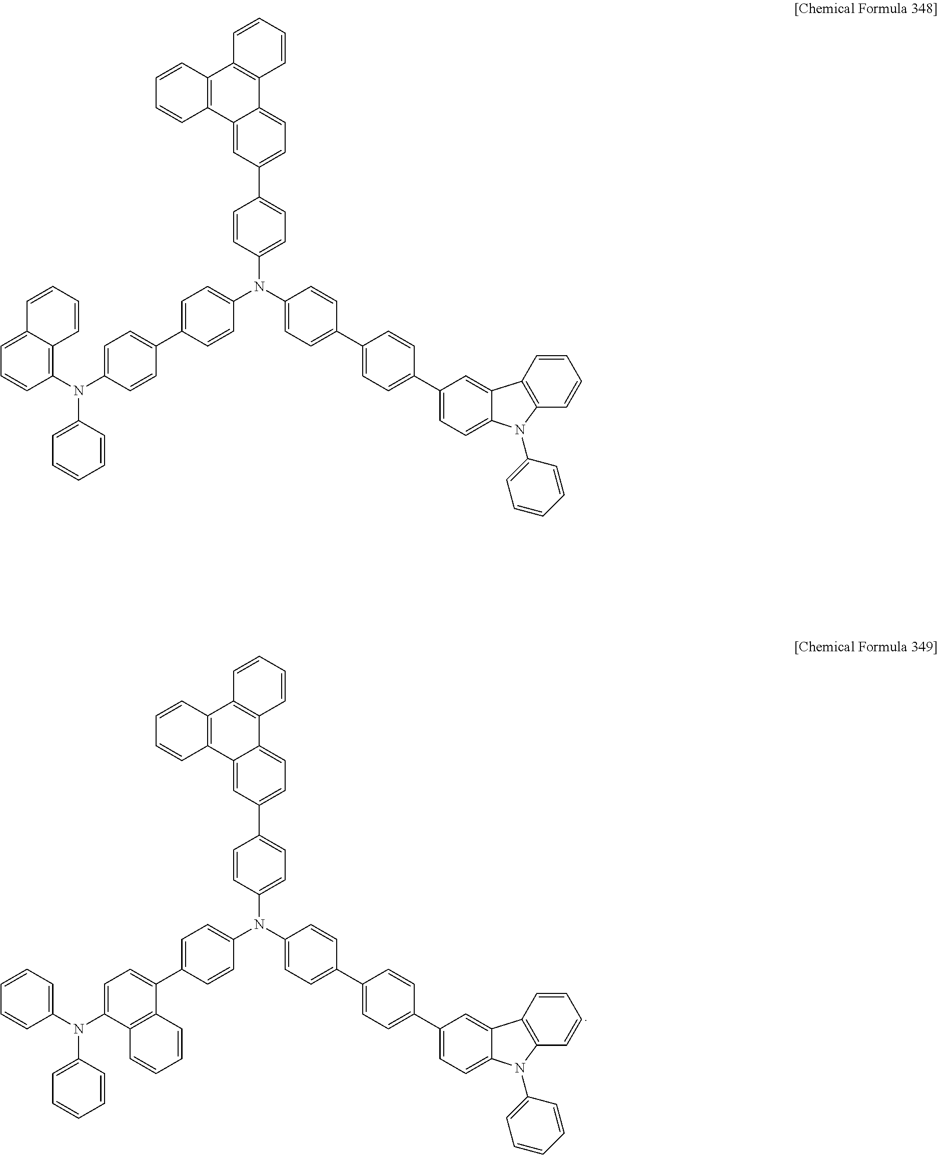 [SMILES: c1ccc(N(c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)c2cccc3ccccc23)cc1 ; c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)c3ccccc23)cc1]

The organic photoelectric device may be selected from the group of an organic light emitting diode, an organic solar cell, an organic transistor, an organic photo conductor drum, and an organic memory device.

Embodiments are also directed to an organic light emitting diode, including an anode, a cathode, and an organic thin layer between the anode and the cathode, the organic thin layer including a compound for an organic photoelectric device according to an embodiment.

The organic thin layer may include one or more of an emission layer, a hole transport layer (HTL), a hole injection layer (HIL), an electron transport layer (ETL), an electron injection layer (EIL), or a hole blocking layer.

The compound for an organic photoelectric device may be included in a hole transport layer (HTL), or a hole injection layer (HIL).

The compound for an organic photoelectric device may be included in an emission layer.

The compound for an organic photoelectric device may be a phosphorescent or fluorescent host material in an emission layer.

The compound for an organic photoelectric device may be a fluorescent blue dopant material in an emission layer.

Embodiments are also directed to a display device including an organic light emitting diode according to an embodiment.

### BRIEF DESCRIPTION OF THE DRAWINGS

Features will become apparent to those of skill in the art by describing in detail example embodiments with reference to the attached drawings in which:

FIGS. 1 to 5 illustrate cross-sectional views showing organic light emitting diodes according to various embodiments including a compound for an organic photoelectric device according to an embodiment.

FIG. 6 shows data of thermal stability of a compound according to Example 2.

FIG. 7 shows thermal decomposition temperatures of compounds according to Examples 2 and 3 and a compound HT2 according to Comparative Example 3.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey example implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when specific definition is not otherwise provided, the term “substituted” refers to one substituted with a C1 to C30 alkyl group, a C1 to C10 alkylsilyl group, a C3 to C30 cycloalkyl group, a C6 to C30 aryl group, a C1 to C10 alkoxy group, a fluoro group, a C1 to C10 trifluoro alkyl group such as trifluoromethyl group, or a cyano group, instead of hydrogen of a compound.

As used herein, when specific definition is not otherwise provided, the term “hetero” refers to one including 1 to 3 hetero atoms selected from the group of N, O, S, and P, and remaining carbons in one functional group.

As used herein, when a definition is not otherwise provided, the term “combination thereof” refers to at least two substituents bound to each other by a linker, or at least two substituents condensed to each other.

In the specification, when a definition is not otherwise provided, the term “alkyl group” may refer to “a saturated group” without any alkene group or alkyne group; or “an unsaturated alkyl group” with at least one alkene group or alkyne group. The “alkene group” may refer to a substituent of at least one carbon-carbon double bond of at least two carbons, and the “alkyne group” may refer to a substituent of at least one carbon-carbon triple bond of at least two carbons. The alkyl group may be branched, linear, or cyclic. The alkyl group may be a C1 to C20 alkyl group, and specifically a C1 to C6 lower alkyl group, a C7 to C10 medium-sized alkyl group, or a C11 to C20 higher alkyl group. For example, a C1 to C4 alkyl group may have 1 to 4 carbon atoms and may be selected from the group of methyl, ethyl, propyl, iso-propyl, n-butyl, iso-butyl, sec-butyl, and t-butyl.

Typical examples of an alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

The term “aromatic group” may refer to a substituent including all element of the cycle having p-orbitals which form conjugation. Examples may include an aryl group and a heteroaryl group. The “aryl group” may refer to a monocyclic or fused ring polycyclic (i.e., rings sharing adjacent pairs of carbon atoms) substituent. The term “heteroaryl group” may refer to an aryl group including 1 to 3 hetero atoms selected from the group of N, O, S, and P, and remaining carbons in one functional group. The aryl group may be a fused ring cyclic group where each cycle may include the 1 to 3 heteroatoms. “Spiro structure” may refer to a plurality of cyclic structures having a contact point of one carbon. The spiro structure may include a compound having a spiro structure or a substituent having a spiro structure.

A compound for an organic photoelectric device according to an embodiment includes a core structure where one of three substituents of an amine compound is a triphenylenyl group, and another substituent is a carbazolyl group or a carbazolyl group derivative. In this specification, the carbazolyl group derivative may refer to a substituent of a carbazolyl group where NR′ is O, S or P. The core structure may have excellent hole properties due to the triphenylenyl group, and carbazolyl group or carbazolyl group derivative. The compound may act as a light emitting host with a dopant in an emission layer.

According to an embodiment, the compound for an organic photoelectric device may include a core part which may include a arylamine part and various substituents substituting the core part. In the core structure, the triphenylenyl group, and carbazolyl group or carbazolyl group derivative may be substituted. The compound may have various energy band gaps. The compound may be used in, e.g., a hole injection layer (HIL) and transport layer, or an emission layer.

The compound may have an energy level depending on the substituents. The compound may enhance a hole transport capability of an organic photoelectric device, may enhance efficiency and driving voltage, may exhibit excellent electrochemical and thermal stability, and may enhance a life-span characteristic during the operation of the organic photoelectric device.

According to an example embodiment, a compound for an organic photoelectric device, the compound being represented by the following Chemical Formula 1, is provided.

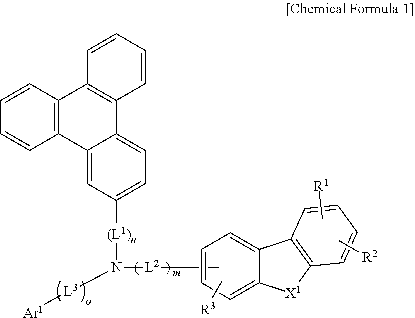

In Chemical Formula 1, L¹ to L³ may each independently be selected from the group of a single bond, a substituted or unsubstituted C2 to C6 alkenylene group, a substituted or unsubstituted C2 to C6 alkynylene group, a substituted or unsubstituted C6 to C30 arylene group and a substituted or unsubstituted C2 to C30 heteroarylene group, and n, m, and o may each independently be integers of 1 to 4. L¹ to L³ may increase a triplet energy band gap by controlling the total π-conjugation length of the compound, which may be useful when applied to the emission layer of organic photoelectric device as phosphorescent host.

X¹ may be selected from the group of NR′, O, S, and P, and R may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group. The carbazolyl group or carbazolyl derivative of X¹ may enhance hole properties and bipolar characteristics of the compound.

Ar¹ may be a substituted or unsubstituted C6 to C30 aryl group or a substituted or unsubstituted C2 to C30 heteroaryl group. Examples of the Ar¹ may be selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group. A combination of the substituents may provide a compound having excellent thermal stability and/or oxidation resistance. A combination of the substituents may provide a bipolar structure, which may enhance transporting capability of holes and electrons and enhance luminous efficiency and performance of a device.

R¹ to R³ may each independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

The substituents may be selected to provide a compound having a bulky structure and thus lower crystallinity. A compound having lower crystallinity may enhance the life-span of a device.

According to another example embodiment, a compound for an organic photoelectric device, the compound being represented by the following Chemical Formula 2, is provided.

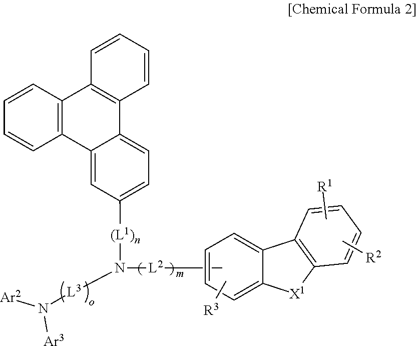

In Chemical Formula 2, L¹ to L³, n, m, o, X¹, and R¹ to R³ are the same as described in the above Chemical Formula 1 and thus details thereof will not be repeated.

In Chemical Formula 2, Ar² and Ar^{a} may each independently be selected from the group of a substituted or unsubstituted C1 to C6 alkyl group, a substituted or unsubstituted C6 to C30 aryl group, and a substituted or unsubstituted C2 to C30 heteroaryl group. The compound of the above Chemical Formula 2 includes an amine substituent, NAr²Ar³. The amine substituent may decrease gaps between HOMO levels of an electrode and a hole injection layer (HIL) and may enable hole injection and transport from an electrode and a hole injection layer (HIL).

Specific examples of Ar¹ and Ar² may be selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group.

According to another example embodiment, a compound for an organic photoelectric device, the compound being represented by the following Chemical Formula 3, is provided.

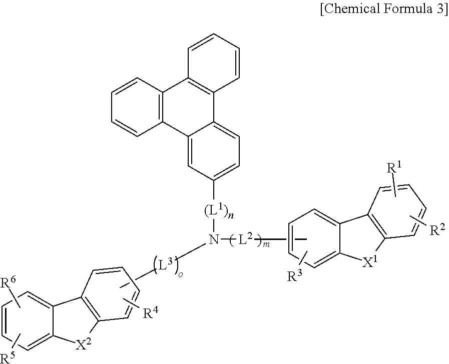

In Chemical Formula 3, L¹ to L³, n, m, and o are the same as described in the above Chemical Formula 1 and thus details thereof will not be repeated.

X¹ and X² may each independently be selected from the group of NR′, O, S, and P, and each R′ may independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

The compound of the above Chemical Formula 3 includes a carbazolyl group or carbazolyl group derivative, which may enhance additional hole properties and bipolar characteristics.

R¹ to R⁶ may each independently be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group. R¹ to R⁶ may be the same as R¹ to R³ in Chemical Formula 1.

X¹ and X² may be NR′, and R′ may be selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group. X¹ and X² may be NR′ which provides a carbazolyl group. When two carbazolyl groups are present, hole transport capability may be increased, and this electric power efficiency and life-span of a device may be enhanced.

The compound represented by the above Chemical Formula 1 may be represented by, e.g., one of the following Chemical Formulae 4 to 39.

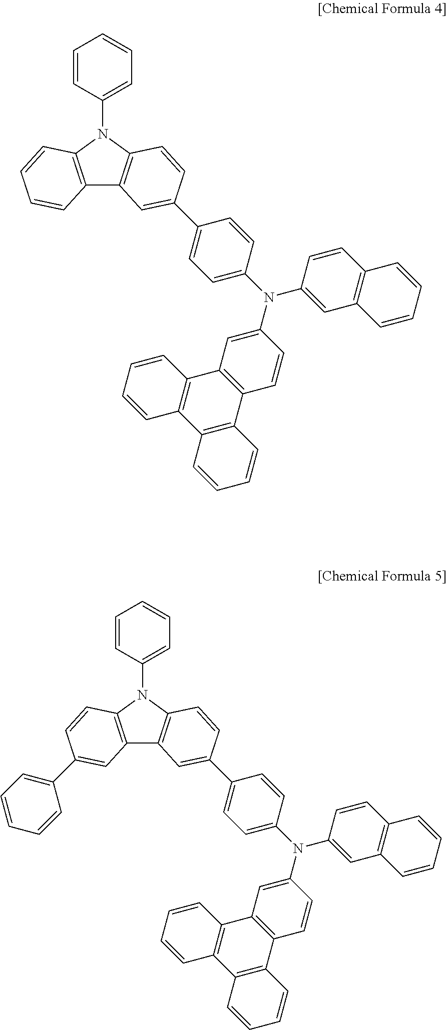 [SMILES: c1ccc(-n2c3ccccc3c3cc(-c4ccc(N(c5ccc6ccccc6c5)c5ccc6c7ccccc7c7ccccc7c6c5)cc4)ccc32)cc1 ; c1ccc(-c2ccc3c(c2)c2cc(-c4ccc(N(c5ccc6ccccc6c5)c5ccc6c7ccccc7c7ccccc7c6c5)cc4)ccc2n3-c2ccccc2)cc1]

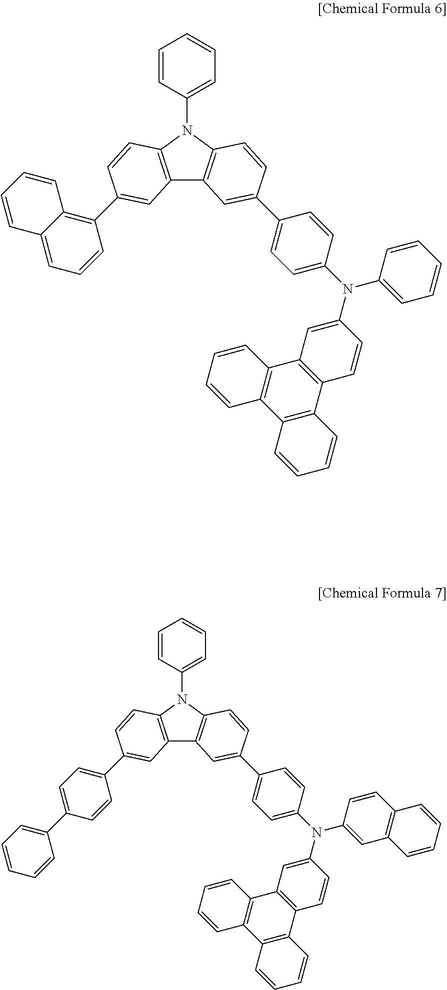 [SMILES: c1ccc(N(c2ccc(-c3ccc4c(c3)c3cc(-c5cccc6ccccc56)ccc3n4-c3ccccc3)cc2)c2ccc3c4ccccc4c4ccccc4c3c2)cc1 ; c1ccc(-c2ccc(-c3ccc4c(c3)c3cc(-c5ccc(N(c6ccc7ccccc7c6)c6ccc7c8ccccc8c8ccccc8c7c6)cc5)ccc3n4-c3ccccc3)cc2)cc1]

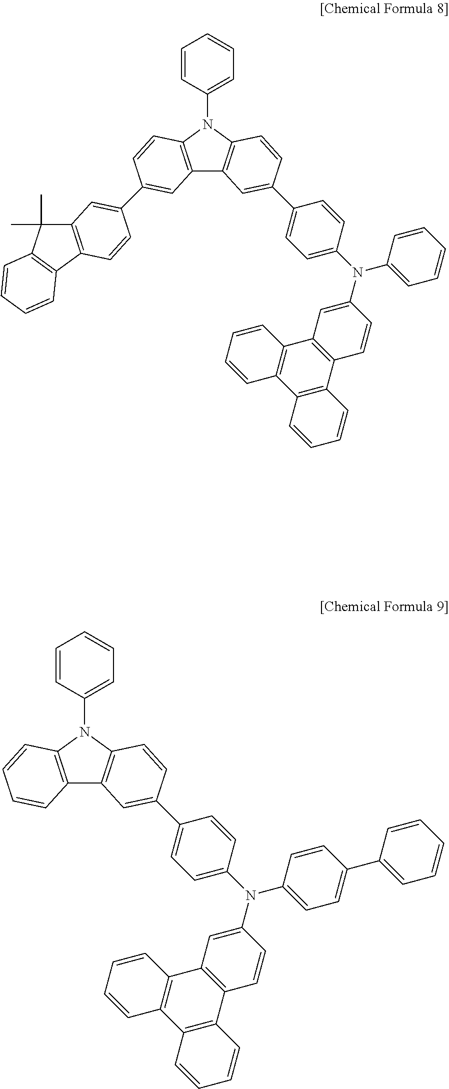 [SMILES: CC1(C)c2ccccc2-c2ccc(-c3ccc4c(c3)c3cc(-c5ccc(N(c6ccccc6)c6ccc7c8ccccc8c8ccccc8c7c6)cc5)ccc3n4-c3ccccc3)cc21 ; c1ccc(-c2ccc(N(c3ccc(-c4ccc5c(c4)c4ccccc4n5-c4ccccc4)cc3)c3ccc4c5ccccc5c5ccccc5c4c3)cc2)cc1]

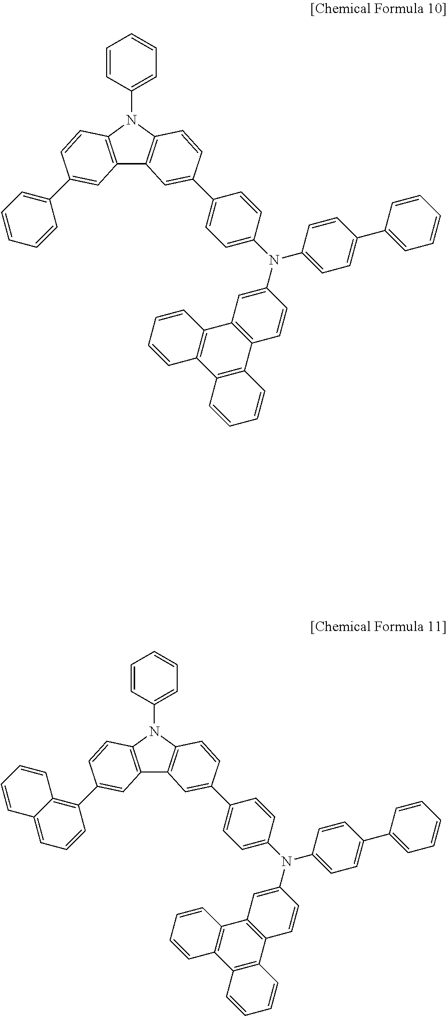 [SMILES: c1ccc(-c2ccc(N(c3ccc(-c4ccc5c(c4)c4cc(-c6ccccc6)ccc4n5-c4ccccc4)cc3)c3ccc4c5ccccc5c5ccccc5c4c3)cc2)cc1 ; c1ccc(-c2ccc(N(c3ccc(-c4ccc5c(c4)c4cc(-c6cccc7ccccc67)ccc4n5-c4ccccc4)cc3)c3ccc4c5ccccc5c5ccccc5c4c3)cc2)cc1]

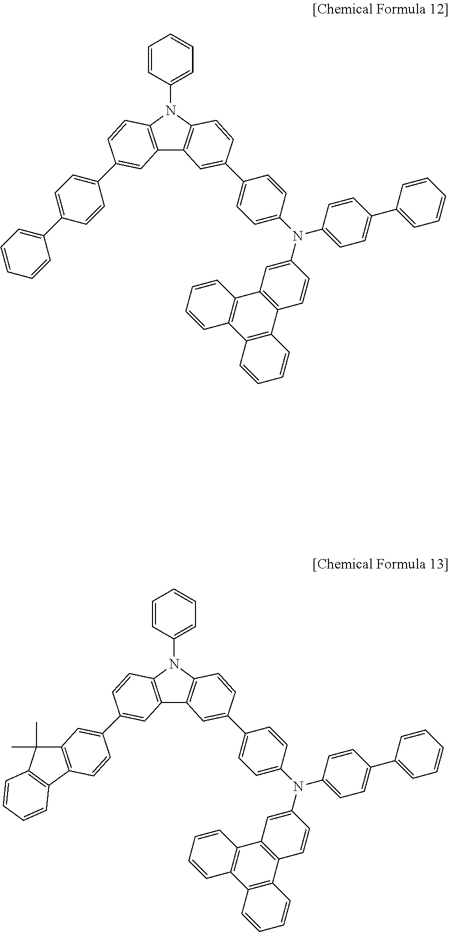 [SMILES: c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc21]

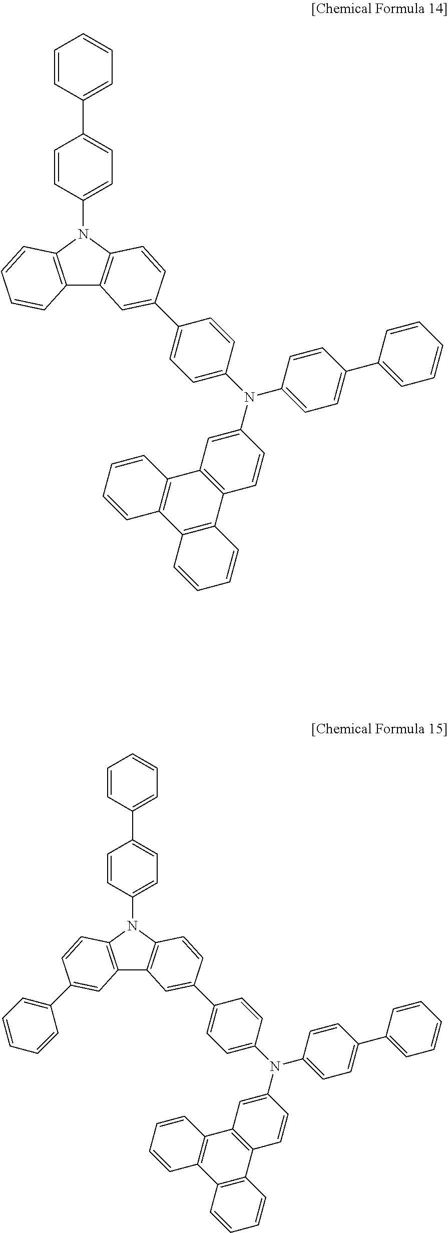 [SMILES: c1ccc(-c2ccc(N(c3ccc(-c4ccc5c(c4)c4ccccc4n5-c4ccc(-c5ccccc5)cc4)cc3)c3ccc4c5ccccc5c5ccccc5c4c3)cc2)cc1 ; c1ccc(-c2ccc(N(c3ccc(-c4ccc5c(c4)c4cc(-c6ccccc6)ccc4n5-c4ccc(-c5ccccc5)cc4)cc3)c3ccc4c5ccccc5c5ccccc5c4c3)cc2)cc1]

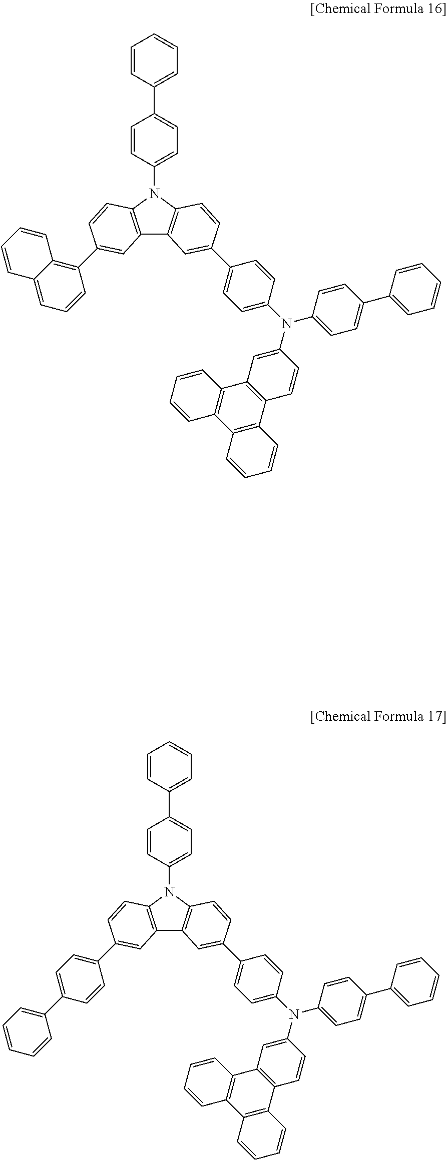 [SMILES: c1ccc(-c2ccc(N(c3ccc(-c4ccc5c(c4)c4cc(-c6cccc7ccccc67)ccc4n5-c4ccc(-c5ccccc5)cc4)cc3)c3ccc4c5ccccc5c5ccccc5c4c3)cc2)cc1 ; c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc2)cc1]

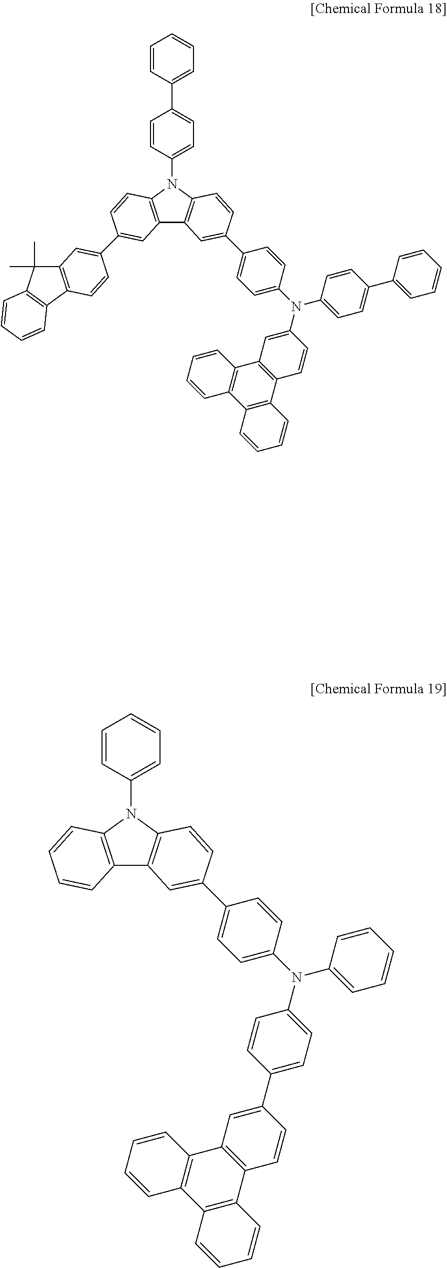 [SMILES: CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc21 ; c1ccc(N(c2ccc(-c3ccc4c5ccccc5c5ccccc5c4c3)cc2)c2ccc(-c3ccc4c(c3)c3ccccc3n4-c3ccccc3)cc2)cc1]

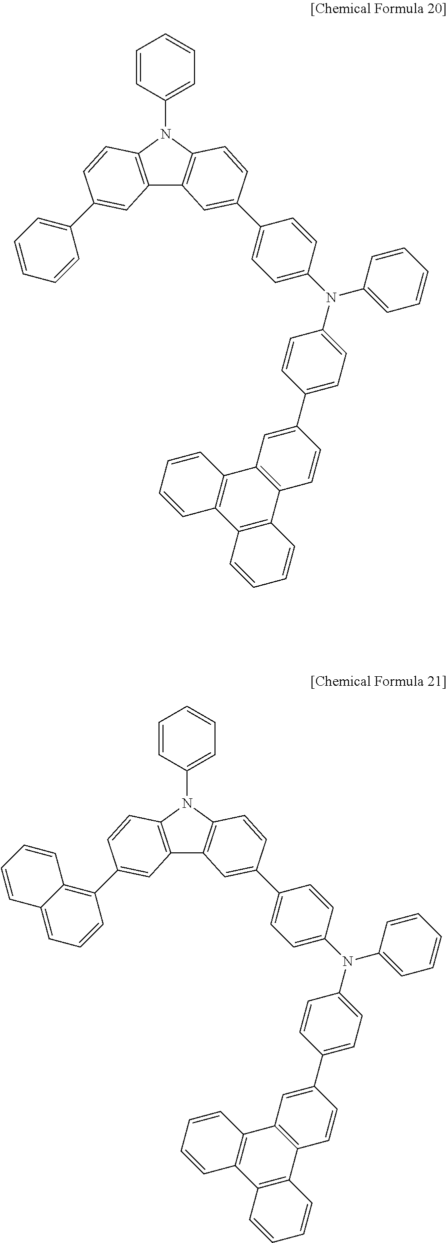 [SMILES: c1ccc(-c2ccc3c(c2)c2cc(-c4ccc(N(c5ccccc5)c5ccc(-c6ccc7c8ccccc8c8ccccc8c7c6)cc5)cc4)ccc2n3-c2ccccc2)cc1 ; c1ccc(N(c2ccc(-c3ccc4c5ccccc5c5ccccc5c4c3)cc2)c2ccc(-c3ccc4c(c3)c3cc(-c5cccc6ccccc56)ccc3n4-c3ccccc3)cc2)cc1]

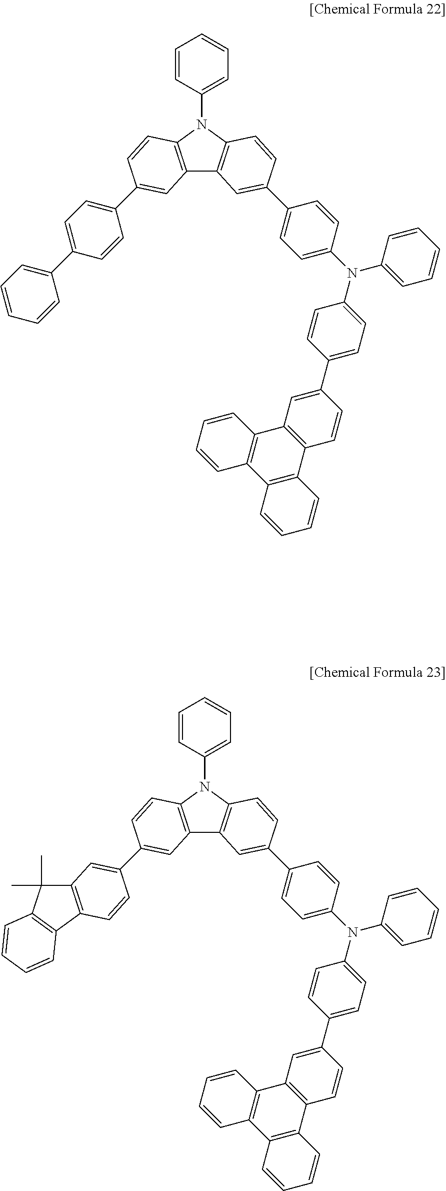 [SMILES: c1ccc(-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc2)cc1 ; CC1(C)c2ccccc2-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc21]

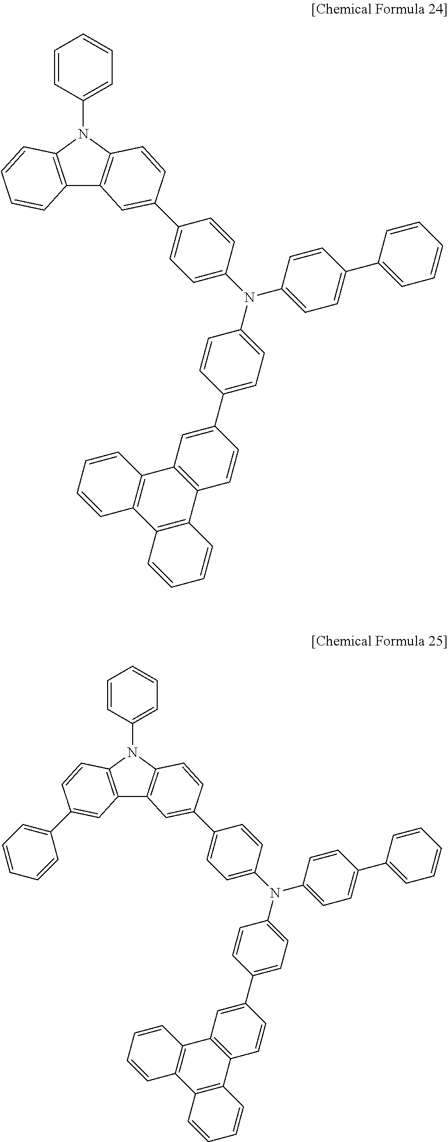 [SMILES: c1ccc(-c2ccc(N(c3ccc(-c4ccc5c6ccccc6c6ccccc6c5c4)cc3)c3ccc(-c4ccc5c(c4)c4ccccc4n5-c4ccccc4)cc3)cc2)cc1 ; c1ccc(-c2ccc(N(c3ccc(-c4ccc5c6ccccc6c6ccccc6c5c4)cc3)c3ccc(-c4ccc5c(c4)c4cc(-c6ccccc6)ccc4n5-c4ccccc4)cc3)cc2)cc1]

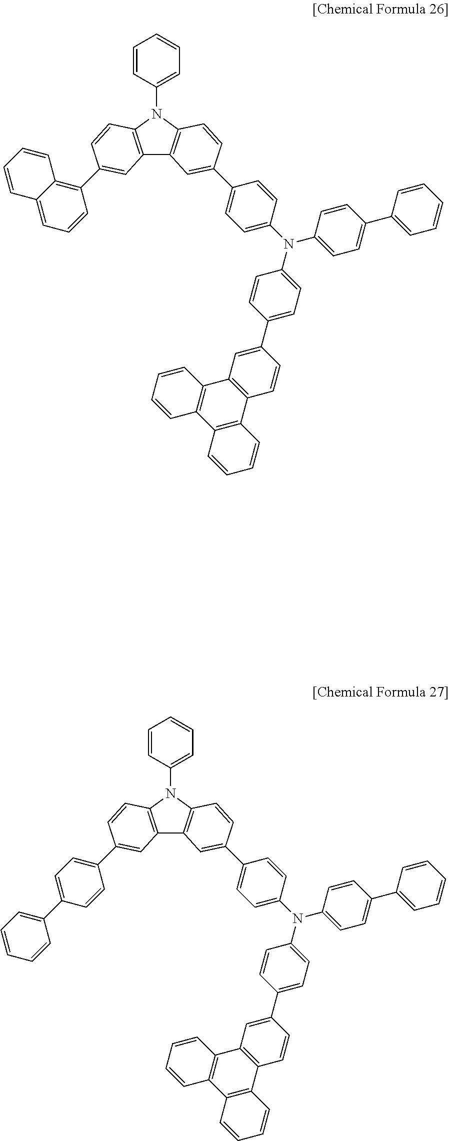 [SMILES: c1ccc(-c2ccc(N(c3ccc(-c4ccc5c6ccccc6c6ccccc6c5c4)cc3)c3ccc(-c4ccc5c(c4)c4cc(-c6cccc7ccccc67)ccc4n5-c4ccccc4)cc3)cc2)cc1 ; c1ccc(-c2ccc(-c3ccc4c(c3)c3cc(-c5ccc(N(c6ccc(-c7ccccc7)cc6)c6ccc(-c7ccc8c9ccccc9c9ccccc9c8c7)cc6)cc5)ccc3n4-c3ccccc3)cc2)cc1]

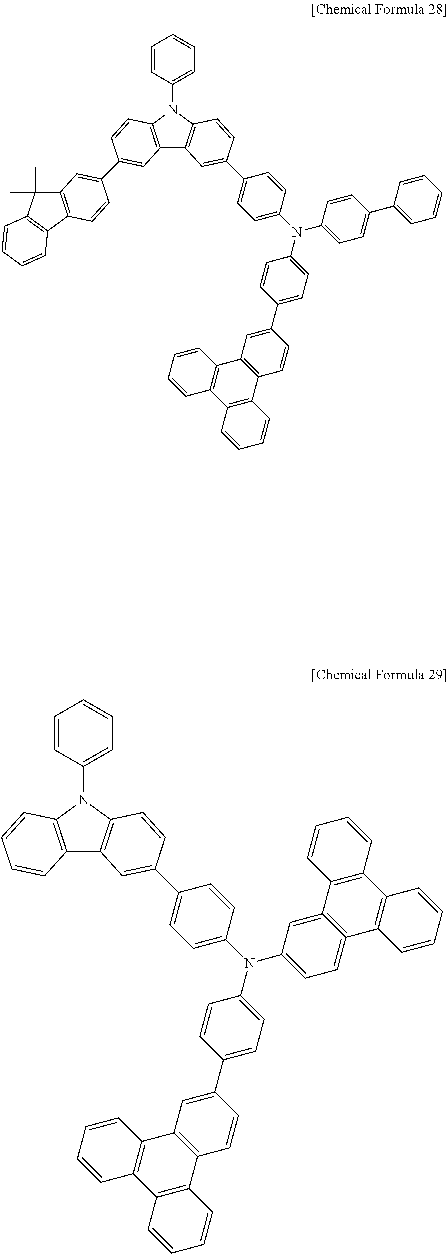

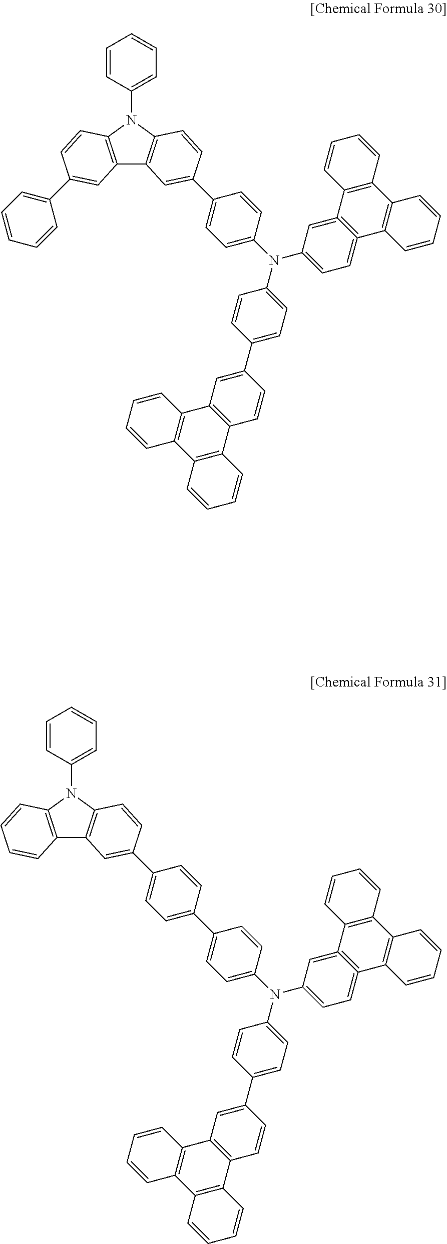

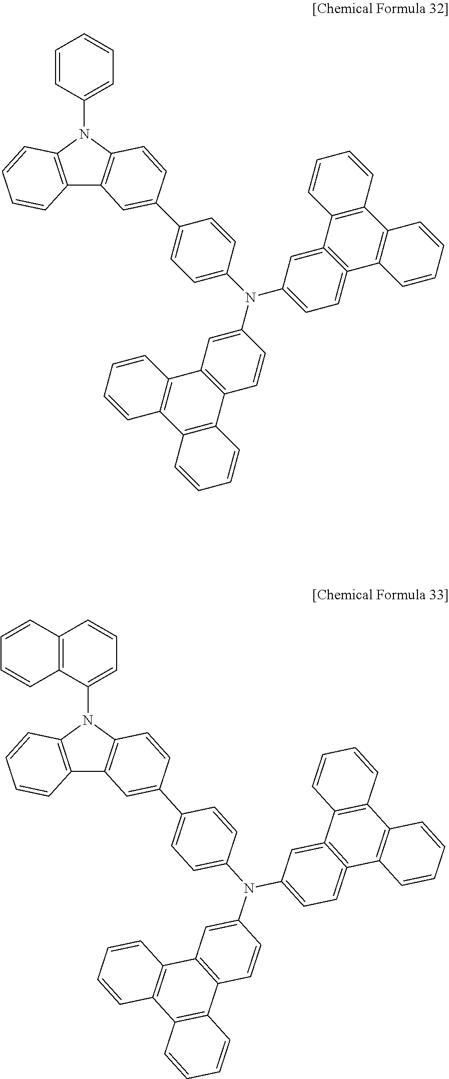

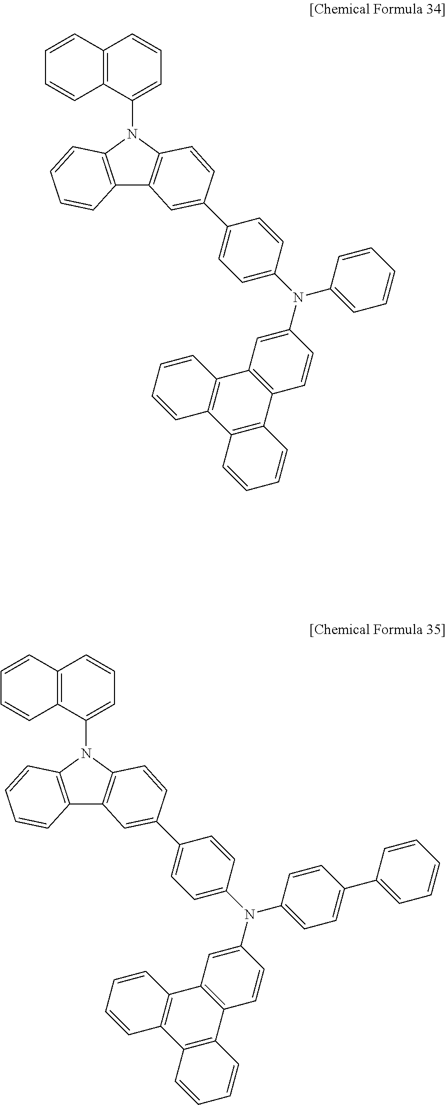

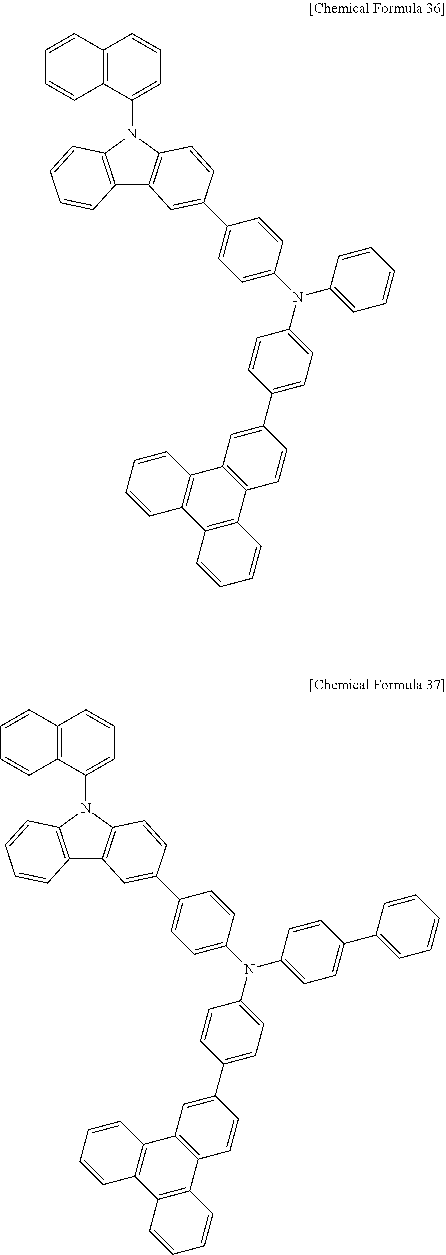

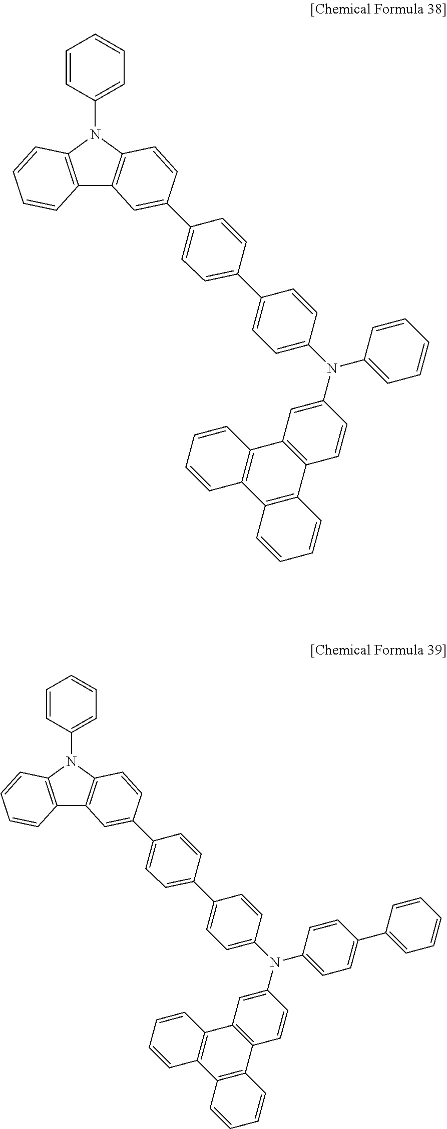 [SMILES: c1ccc(N(c2ccc(-c3ccc(-c4ccc5c(c4)c4ccccc4n5-c4ccccc4)cc3)cc2)c2ccc3c4ccccc4c4ccccc4c3c2)cc1 ; c1ccc(-c2ccc(N(c3ccc(-c4ccc(-c5ccc6c(c5)c5ccccc5n6-c5ccccc5)cc4)cc3)c3ccc4c5ccccc5c5ccccc5c4c3)cc2)cc1]

The compound represented by the above Chemical Formula 1 may be represented by, e.g., one of the following Chemical Formula ad1 or ad2.

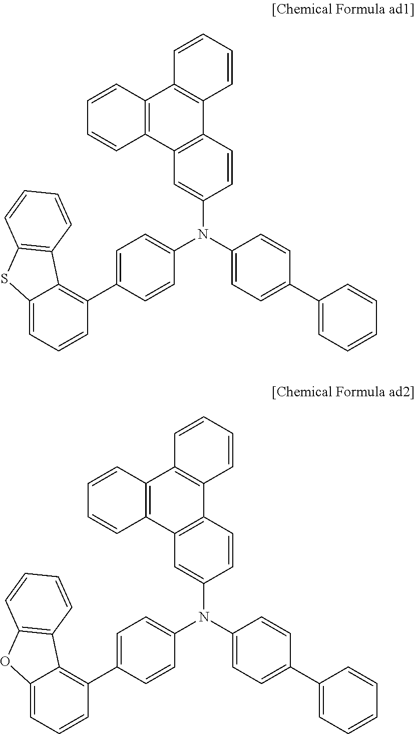 [SMILES: c1ccc(-c2ccc(N(c3ccc(-c4cccc5sc6ccccc6c45)cc3)c3ccc4c5ccccc5c5ccccc5c4c3)cc2)cc1 ; c1ccc(-c2ccc(N(c3ccc(-c4cccc5oc6ccccc6c45)cc3)c3ccc4c5ccccc5c5ccccc5c4c3)cc2)cc1]

The compound represented by the above Chemical Formula 3 may be represented by, e.g., one of the following Chemical Formulae 40 to 106.

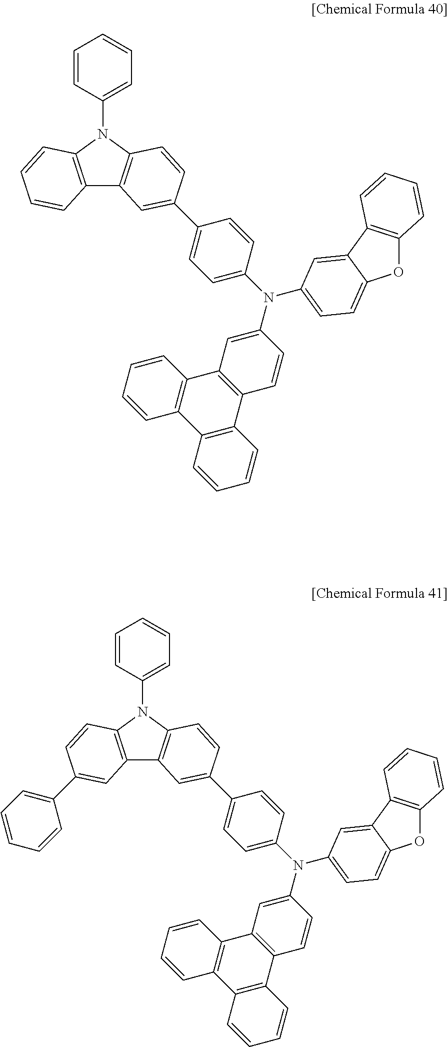 [SMILES: c1ccc(-n2c3ccccc3c3cc(-c4ccc(N(c5ccc6oc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6oc7ccccc7c6c5)c5ccc6c7ccccc7c7ccccc7c6c5)cc4)ccc2n3-c2ccccc2)cc1]

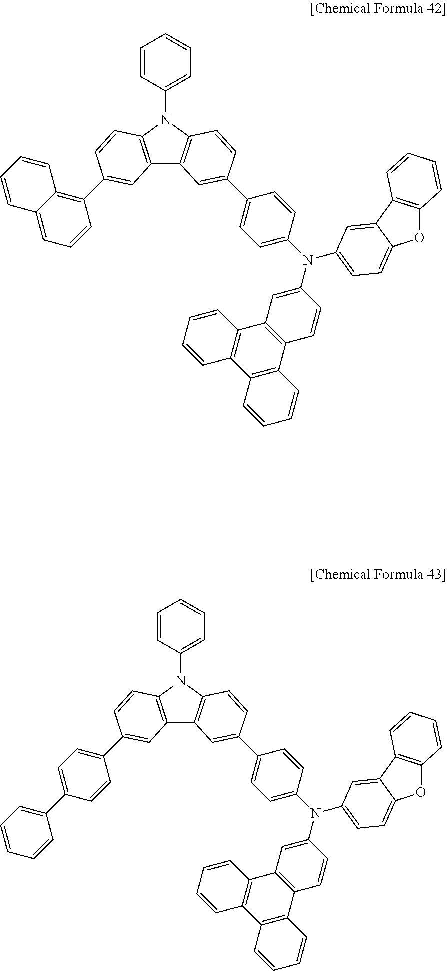 [SMILES: c1ccc(-n2c3ccc(-c4ccc(N(c5ccc6oc7ccccc7c6c5)c5ccc6c7ccccc7c7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc2)cc1]

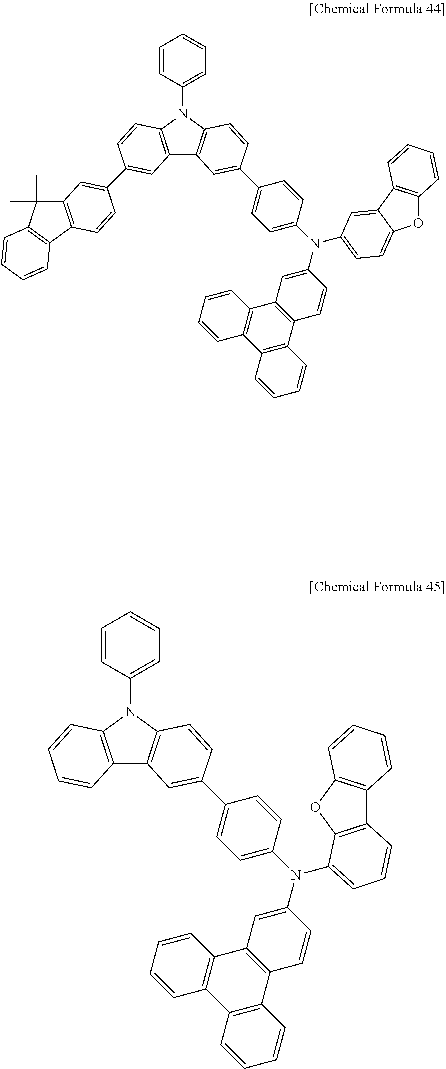 [SMILES: CC1(C)c2ccccc2-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc21 ; c1ccc(-n2c3ccccc3c3cc(-c4ccc(N(c5ccc6c7ccccc7c7ccccc7c6c5)c5cccc6c5oc5ccccc56)cc4)ccc32)cc1]

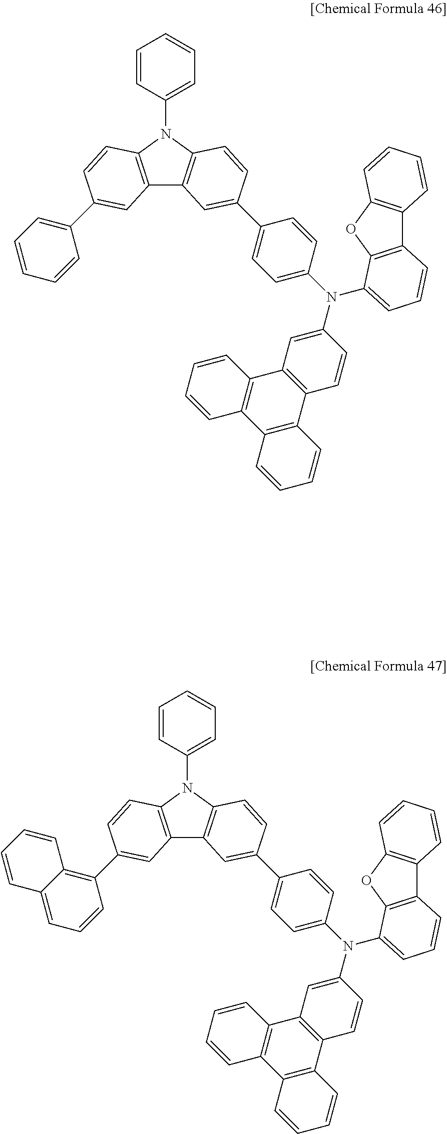 [SMILES: c1ccc(-c2ccc3c(c2)c2cc(-c4ccc(N(c5ccc6c7ccccc7c7ccccc7c6c5)c5cccc6c5oc5ccccc56)cc4)ccc2n3-c2ccccc2)cc1 ; c1ccc(-n2c3ccc(-c4ccc(N(c5ccc6c7ccccc7c7ccccc7c6c5)c5cccc6c5oc5ccccc56)cc4)cc3c3cc(-c4cccc5ccccc45)ccc32)cc1]

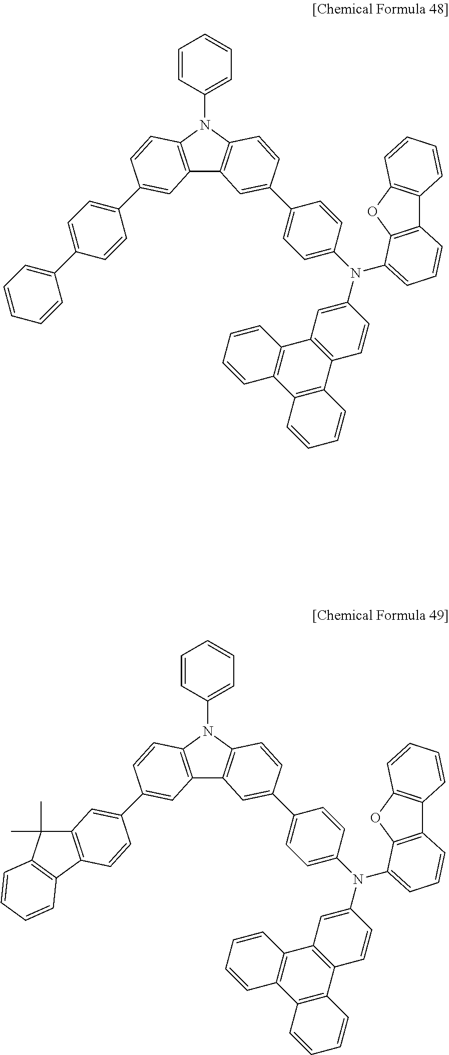

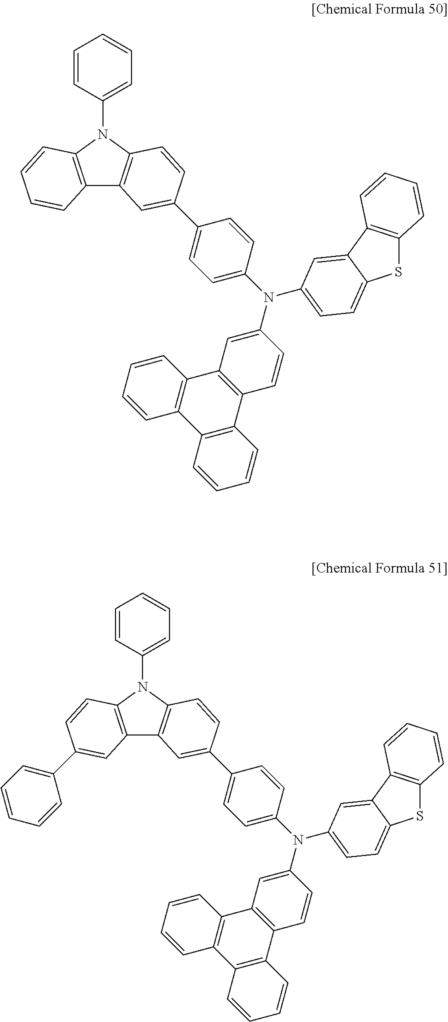 [SMILES: c1ccc(-n2c3ccccc3c3cc(-c4ccc(N(c5ccc6sc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6sc7ccccc7c6c5)c5ccc6c7ccccc7c7ccccc7c6c5)cc4)ccc2n3-c2ccccc2)cc1]

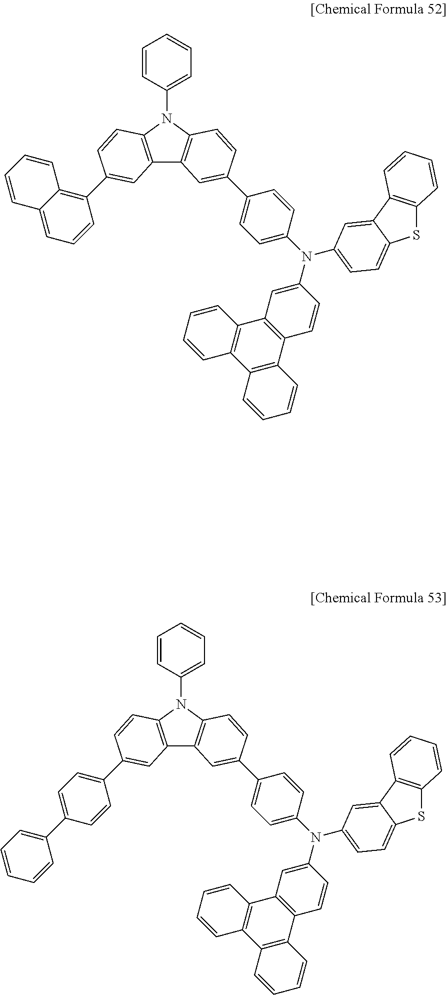 [SMILES: c1ccc(-n2c3ccc(-c4ccc(N(c5ccc6sc7ccccc7c6c5)c5ccc6c7ccccc7c7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc2)cc1]

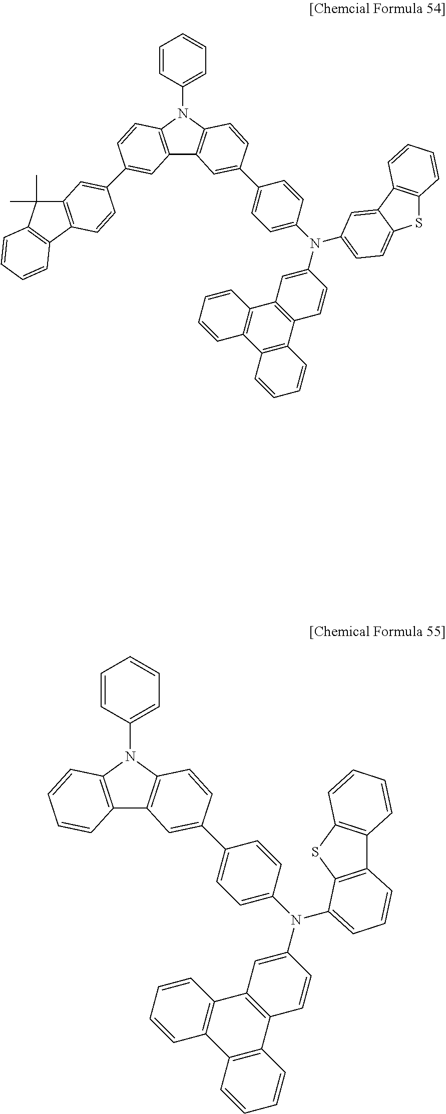 [SMILES: CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc21 ; c1ccc(-n2c3ccccc3c3cc(-c4ccc(N(c5ccc6c7ccccc7c7ccccc7c6c5)c5cccc6c5sc5ccccc56)cc4)ccc32)cc1]

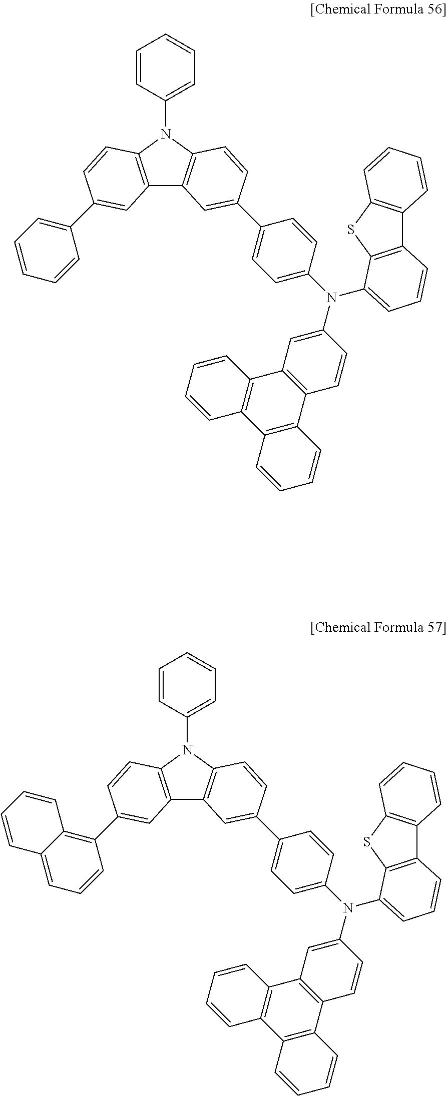 [SMILES: c1ccc(-c2ccc3c(c2)c2cc(-c4ccc(N(c5ccc6c7ccccc7c7ccccc7c6c5)c5cccc6c5sc5ccccc56)cc4)ccc2n3-c2ccccc2)cc1 ; c1ccc(-n2c3ccc(-c4ccc(N(c5ccc6c7ccccc7c7ccccc7c6c5)c5cccc6c5sc5ccccc56)cc4)cc3c3cc(-c4cccc5ccccc45)ccc32)cc1]

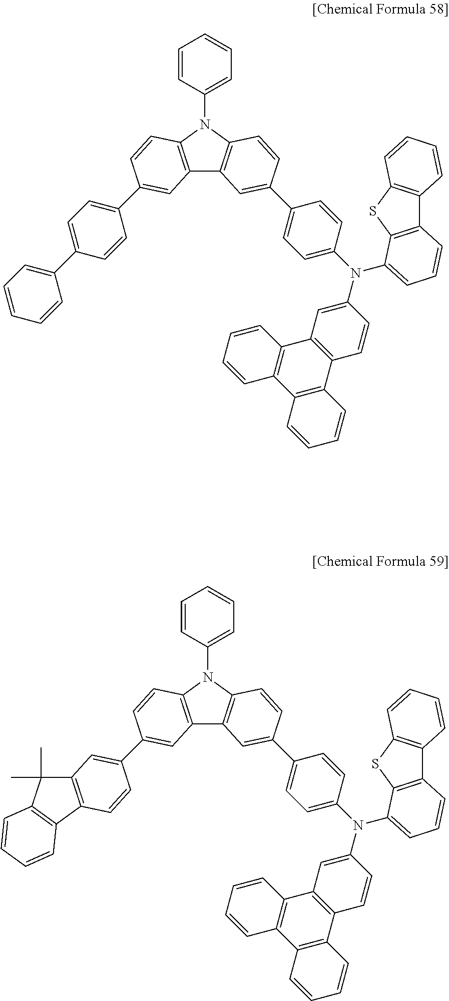 [SMILES: c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc21]

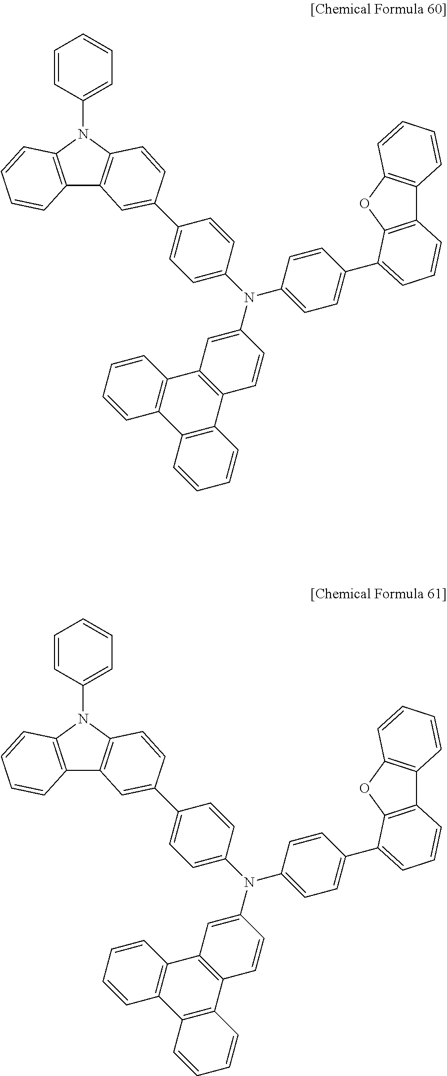 [SMILES: c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1 ; c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1]

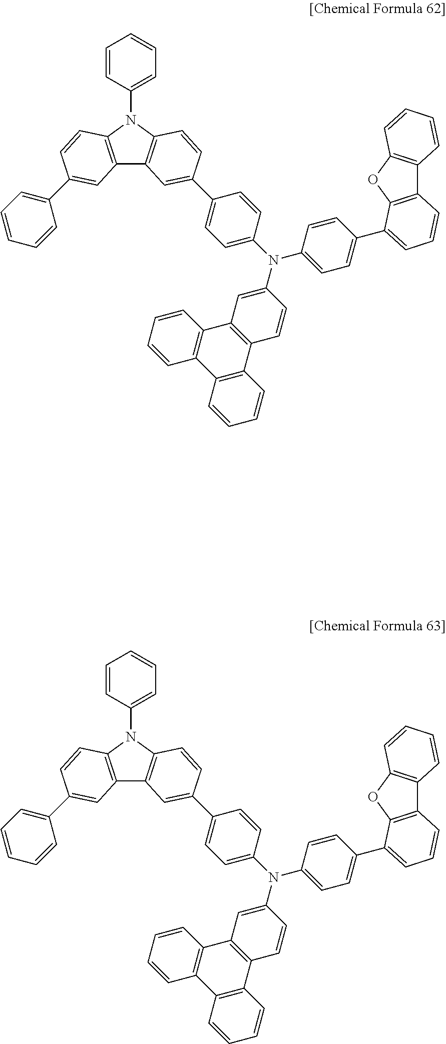 [SMILES: c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1 ; c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1]

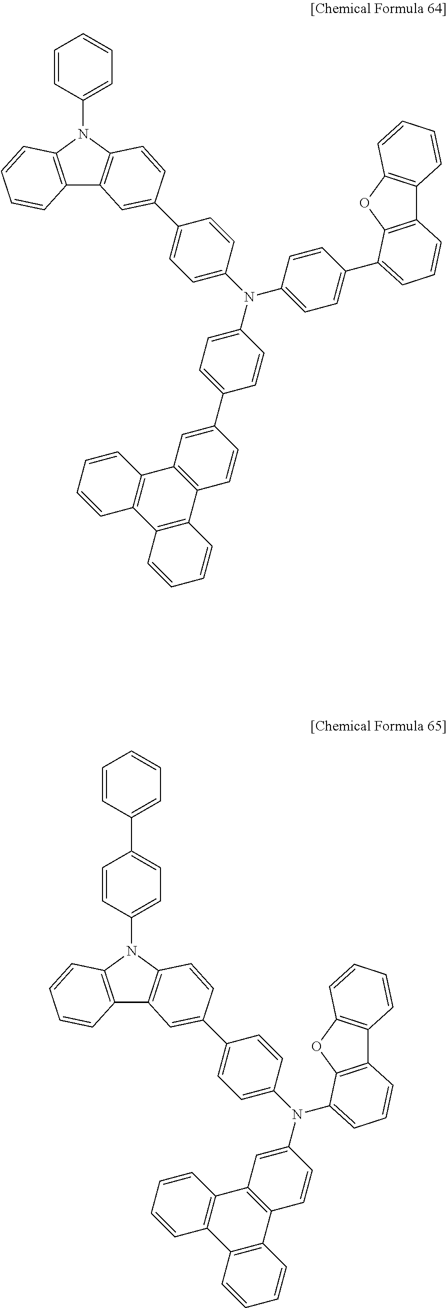 [SMILES: c1ccc(-n2c3ccccc3c3cc(-c4ccc(N(c5ccc(-c6ccc7c8ccccc8c8ccccc8c7c6)cc5)c5ccc(-c6cccc7c6oc6ccccc67)cc5)cc4)ccc32)cc1 ; c1ccc(-c2ccc(-n3c4ccccc4c4cc(-c5ccc(N(c6ccc7c8ccccc8c8ccccc8c7c6)c6cccc7c6oc6ccccc67)cc5)ccc43)cc2)cc1]

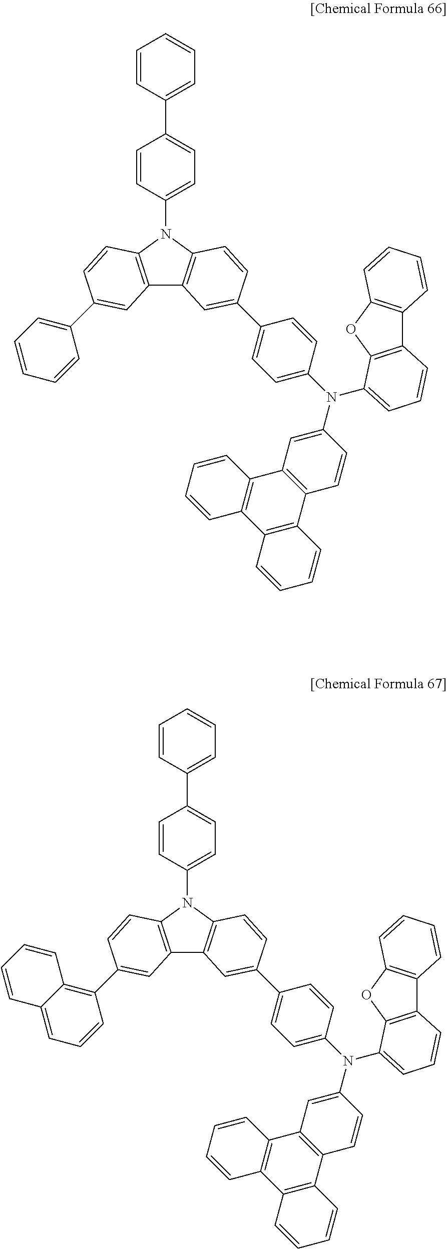 [SMILES: c1ccc(-c2ccc(-n3c4ccc(-c5ccccc5)cc4c4cc(-c5ccc(N(c6ccc7c8ccccc8c8ccccc8c7c6)c6cccc7c6oc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6oc6ccccc67)cc5)cc4c4cc(-c5cccc6ccccc56)ccc43)cc2)cc1]

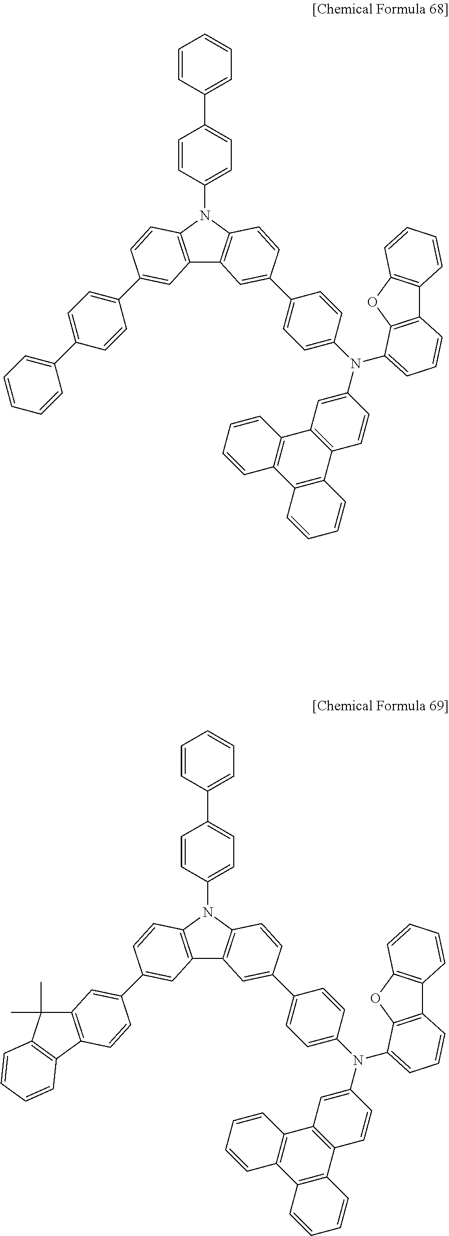 [SMILES: c1ccc(-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc21]

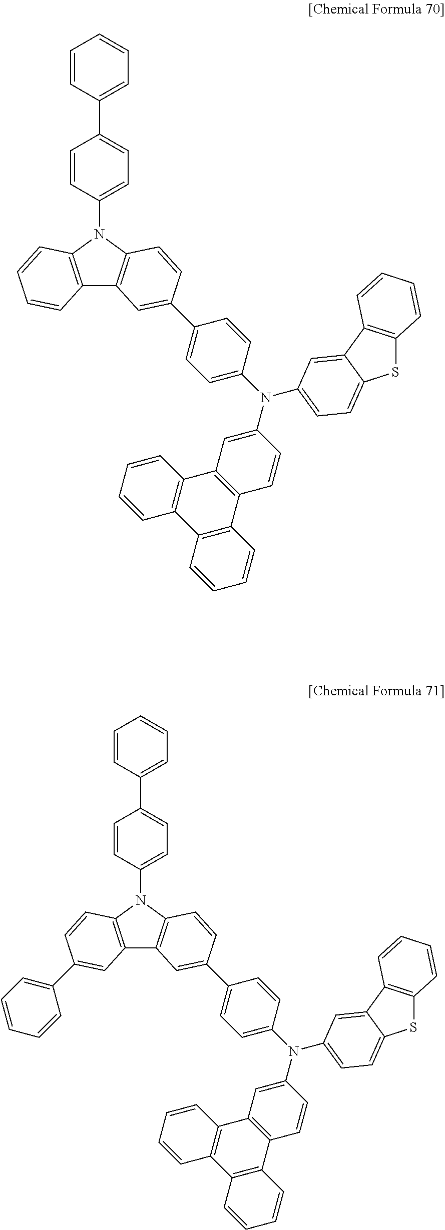 [SMILES: c1ccc(-c2ccc(-n3c4ccccc4c4cc(-c5ccc(N(c6ccc7sc8ccccc8c7c6)c6ccc7c8ccccc8c8ccccc8c7c6)cc5)ccc43)cc2)cc1 ; c1ccc(-c2ccc(-n3c4ccc(-c5ccccc5)cc4c4cc(-c5ccc(N(c6ccc7sc8ccccc8c7c6)c6ccc7c8ccccc8c8ccccc8c7c6)cc5)ccc43)cc2)cc1]

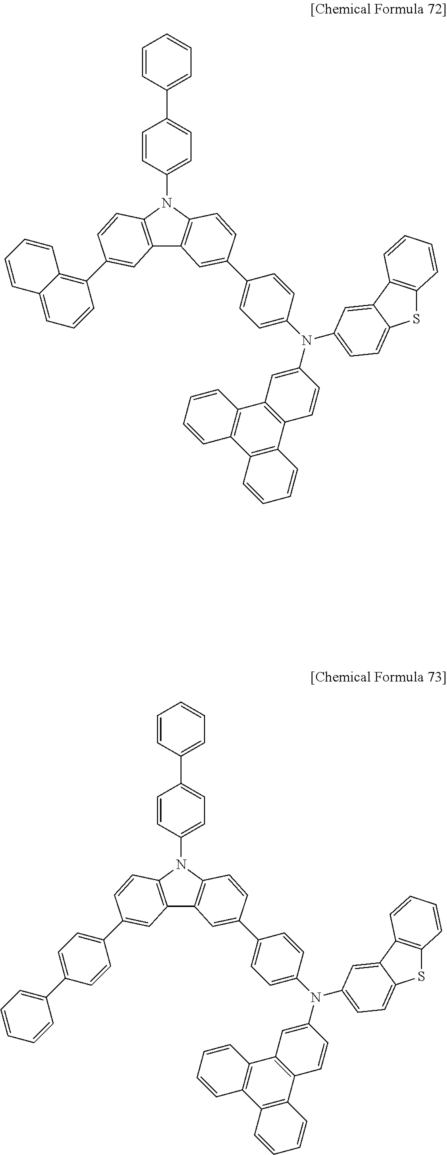 [SMILES: c1ccc(-c2ccc(-n3c4ccc(-c5ccc(N(c6ccc7sc8ccccc8c7c6)c6ccc7c8ccccc8c8ccccc8c7c6)cc5)cc4c4cc(-c5cccc6ccccc56)ccc43)cc2)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc2)cc1]

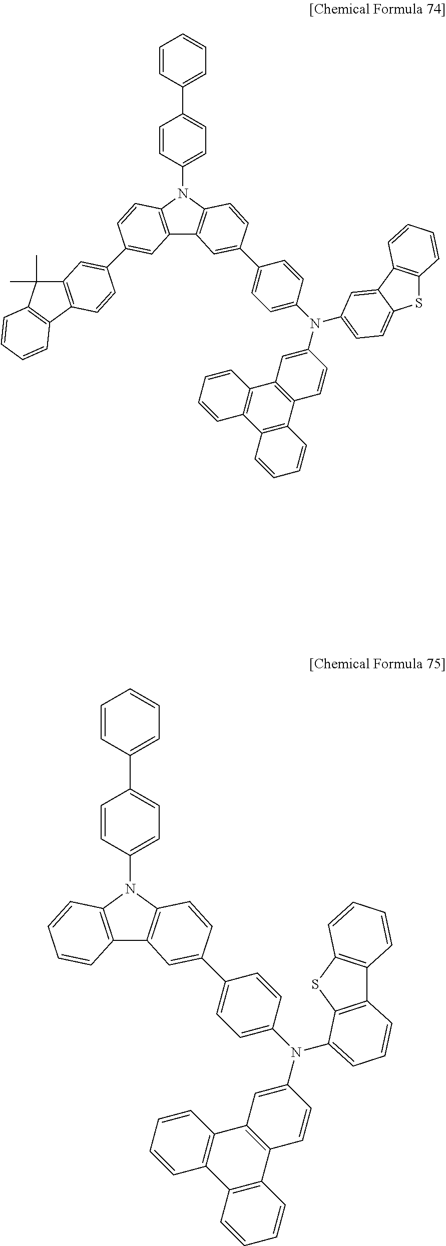 [SMILES: CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc21 ; c1ccc(-c2ccc(-n3c4ccccc4c4cc(-c5ccc(N(c6ccc7c8ccccc8c8ccccc8c7c6)c6cccc7c6sc6ccccc67)cc5)ccc43)cc2)cc1]

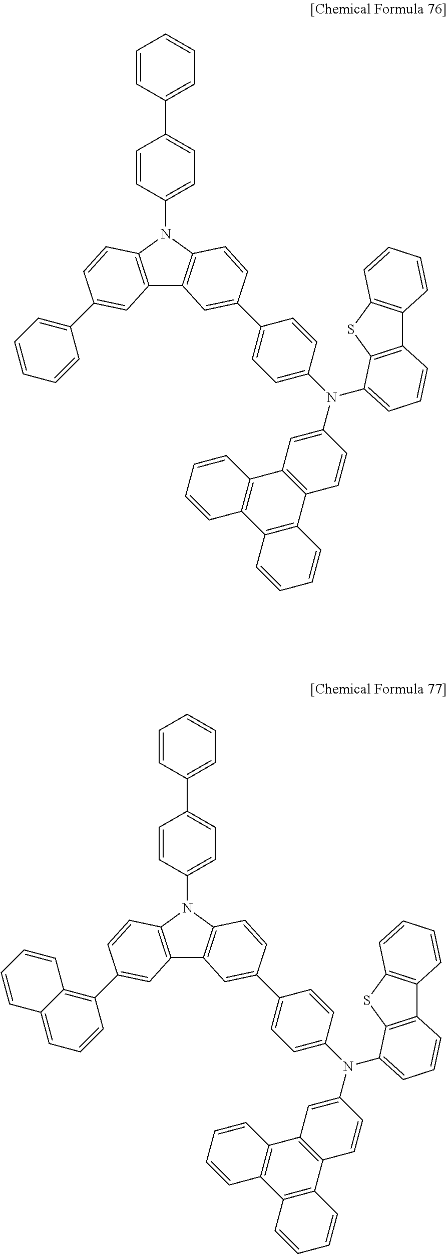 [SMILES: c1ccc(-c2ccc(-n3c4ccc(-c5ccccc5)cc4c4cc(-c5ccc(N(c6ccc7c8ccccc8c8ccccc8c7c6)c6cccc7c6sc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6sc6ccccc67)cc5)cc4c4cc(-c5cccc6ccccc56)ccc43)cc2)cc1]

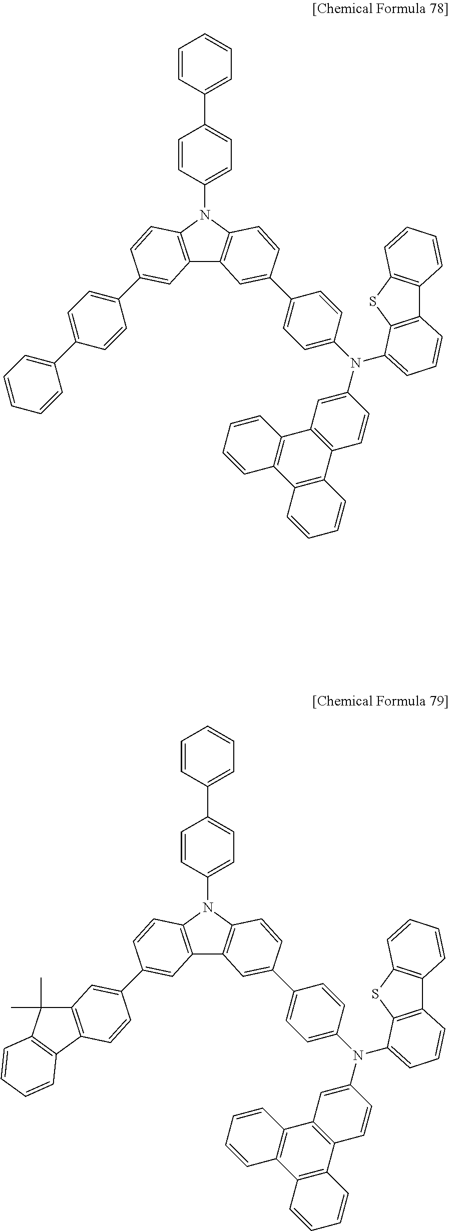 [SMILES: c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc21]

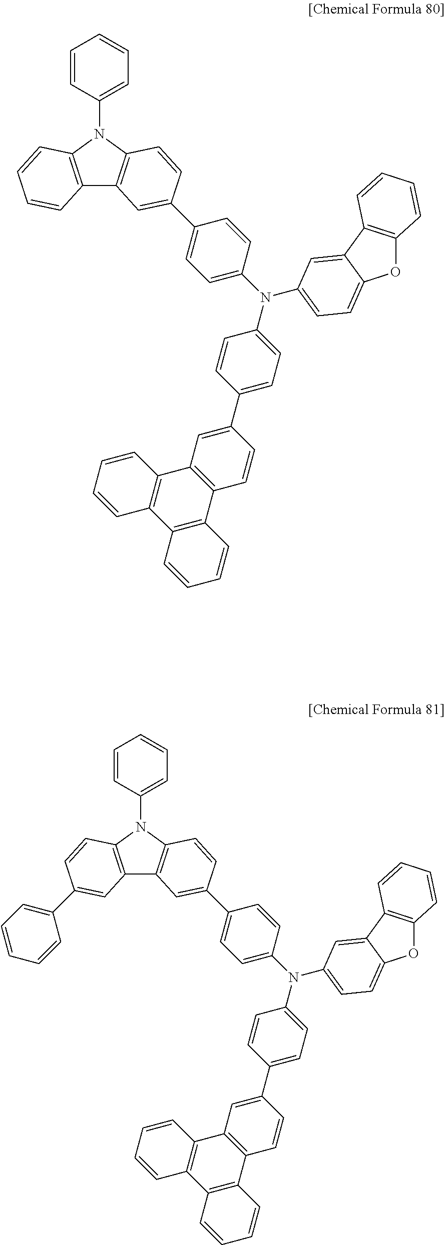 [SMILES: c1ccc(-n2c3ccccc3c3cc(-c4ccc(N(c5ccc(-c6ccc7c8ccccc8c8ccccc8c7c6)cc5)c5ccc6oc7ccccc7c6c5)cc4)ccc32)cc1 ; c1ccc(-c2ccc3c(c2)c2cc(-c4ccc(N(c5ccc(-c6ccc7c8ccccc8c8ccccc8c7c6)cc5)c5ccc6oc7ccccc7c6c5)cc4)ccc2n3-c2ccccc2)cc1]

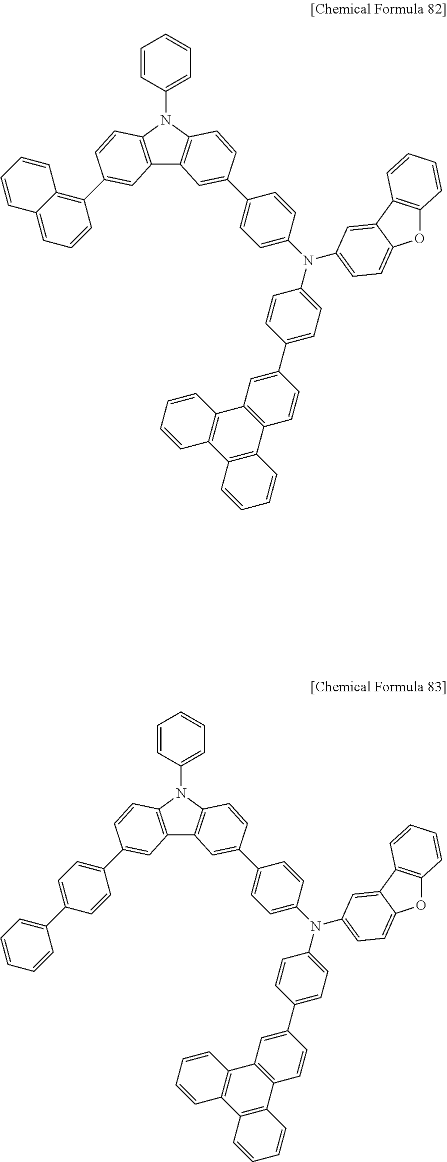 [SMILES: c1ccc(-n2c3ccc(-c4ccc(N(c5ccc(-c6ccc7c8ccccc8c8ccccc8c7c6)cc5)c5ccc6oc7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6ccc7oc8ccccc8c7c6)cc5)ccc3n4-c3ccccc3)cc2)cc1]

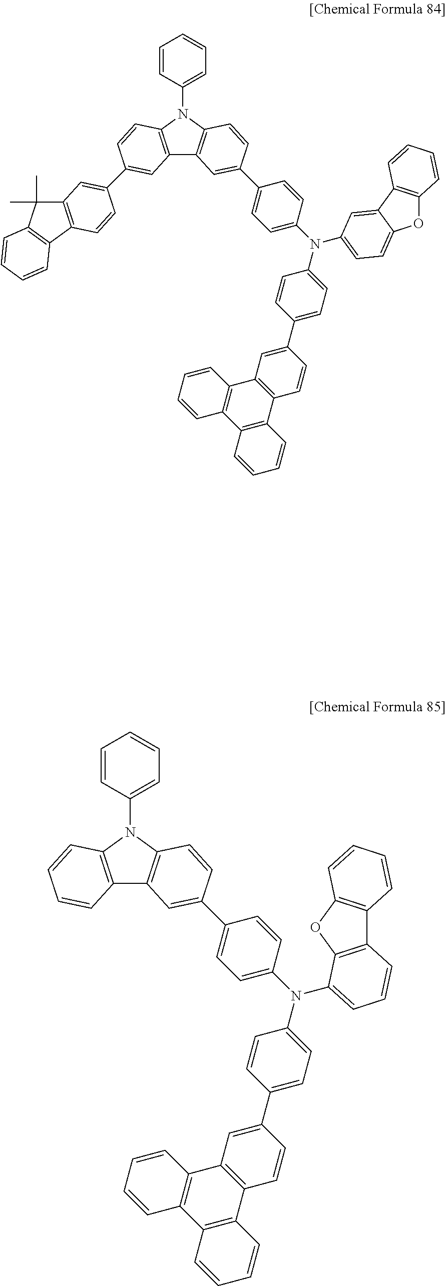 [SMILES: CC1(C)c2ccccc2-c2ccc(-c3ccc4c(c3)c3cc(-c5ccc(N(c6ccc(-c7ccc8c9ccccc9c9ccccc9c8c7)cc6)c6ccc7oc8ccccc8c7c6)cc5)ccc3n4-c3ccccc3)cc21 ; c1ccc(-n2c3ccccc3c3cc(-c4ccc(N(c5ccc(-c6ccc7c8ccccc8c8ccccc8c7c6)cc5)c5cccc6c5oc5ccccc56)cc4)ccc32)cc1]

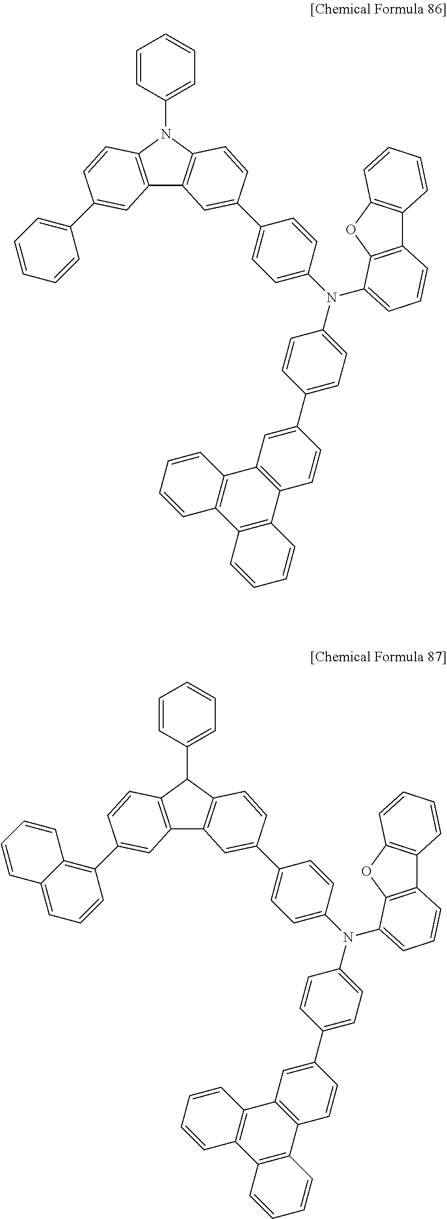 [SMILES: c1ccc(-c2ccc3c(c2)c2cc(-c4ccc(N(c5ccc(-c6ccc7c8ccccc8c8ccccc8c7c6)cc5)c5cccc6c5oc5ccccc56)cc4)ccc2n3-c2ccccc2)cc1 ; c1ccc(C2c3ccc(-c4ccc(N(c5ccc(-c6ccc7c8ccccc8c8ccccc8c7c6)cc5)c5cccc6c5oc5ccccc56)cc4)cc3-c3cc(-c4cccc5ccccc45)ccc32)cc1]

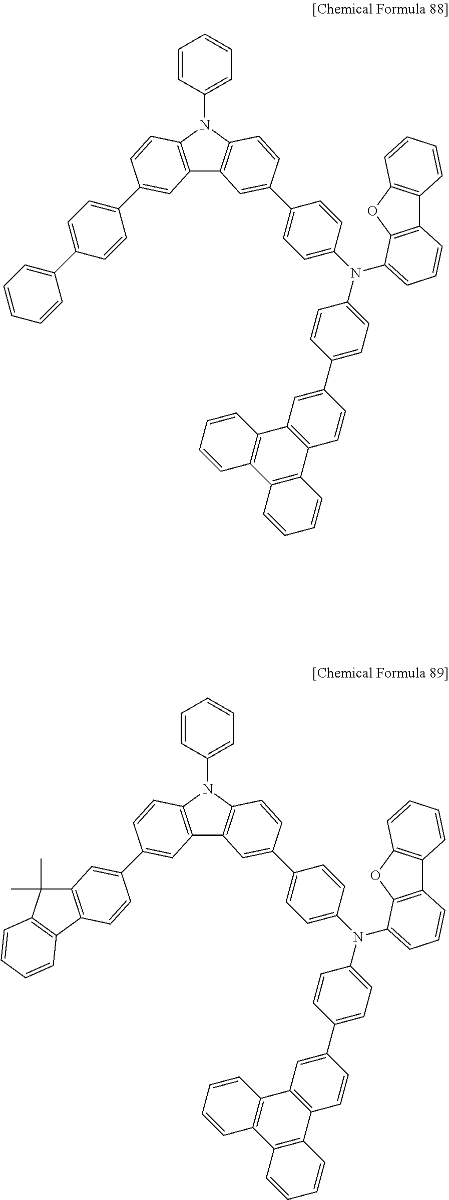 [SMILES: c1ccc(-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc21]

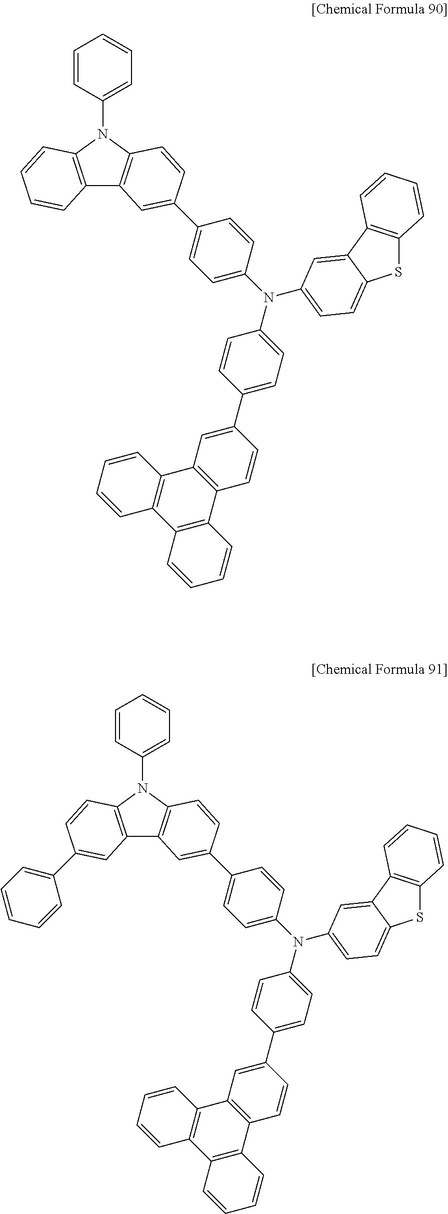 [SMILES: c1ccc(-n2c3ccccc3c3cc(-c4ccc(N(c5ccc(-c6ccc7c8ccccc8c8ccccc8c7c6)cc5)c5ccc6sc7ccccc7c6c5)cc4)ccc32)cc1 ; c1ccc(-c2ccc3c(c2)c2cc(-c4ccc(N(c5ccc(-c6ccc7c8ccccc8c8ccccc8c7c6)cc5)c5ccc6sc7ccccc7c6c5)cc4)ccc2n3-c2ccccc2)cc1]

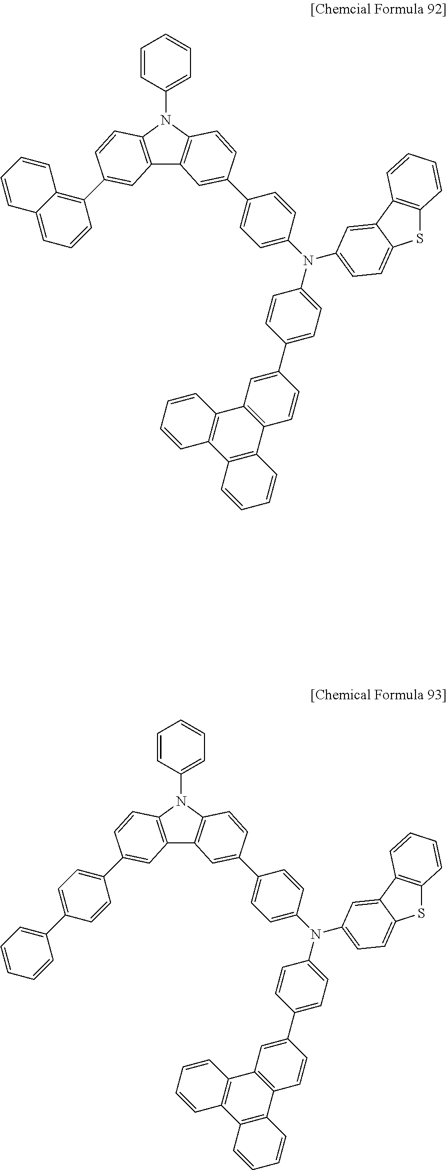 [SMILES: c1ccc(-n2c3ccc(-c4ccc(N(c5ccc(-c6ccc7c8ccccc8c8ccccc8c7c6)cc5)c5ccc6sc7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc2)cc1]

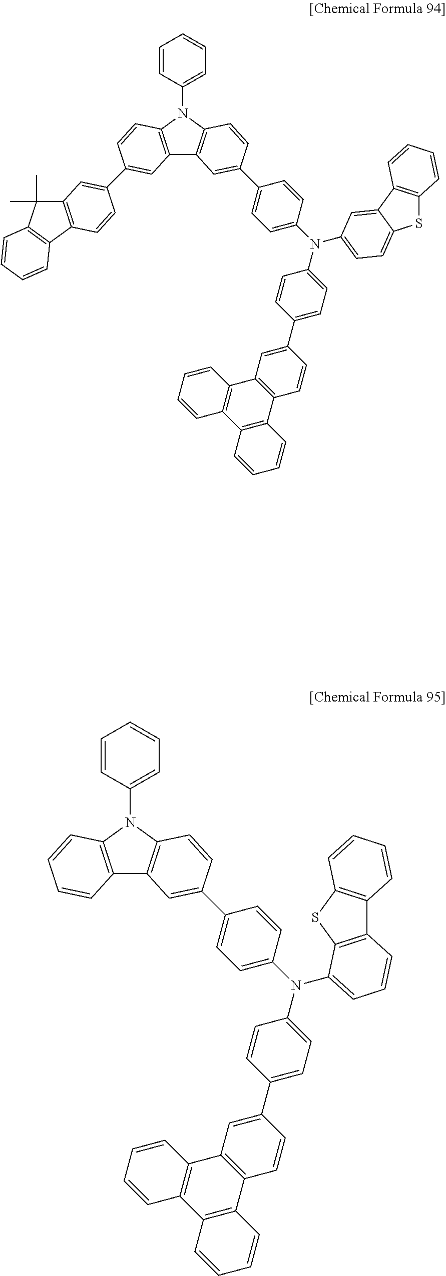 [SMILES: CC1(C)c2ccccc2-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc21 ; c1ccc(-n2c3ccccc3c3cc(-c4ccc(N(c5ccc(-c6ccc7c8ccccc8c8ccccc8c7c6)cc5)c5cccc6c5sc5ccccc56)cc4)ccc32)cc1]

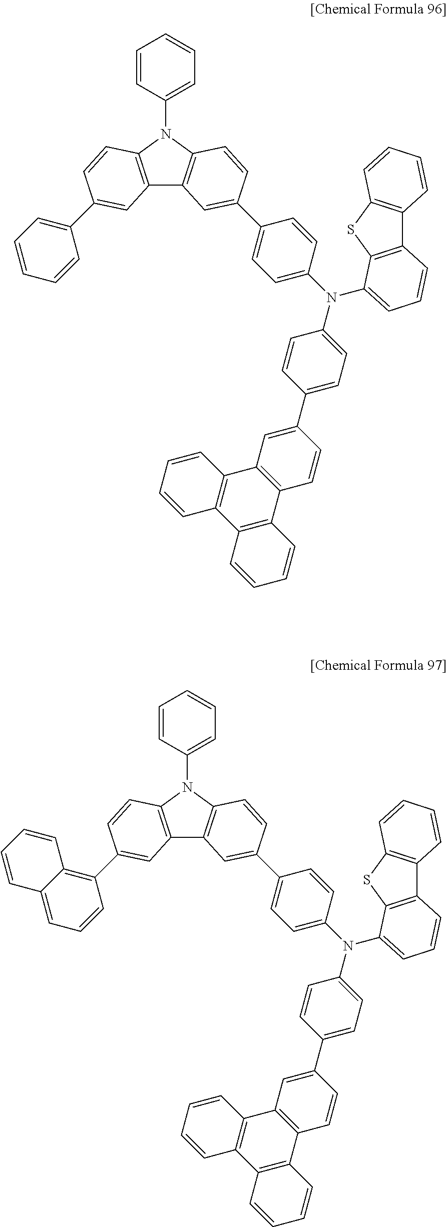 [SMILES: c1ccc(-c2ccc3c(c2)c2cc(-c4ccc(N(c5ccc(-c6ccc7c8ccccc8c8ccccc8c7c6)cc5)c5cccc6c5sc5ccccc56)cc4)ccc2n3-c2ccccc2)cc1 ; c1ccc(-n2c3ccc(-c4ccc(N(c5ccc(-c6ccc7c8ccccc8c8ccccc8c7c6)cc5)c5cccc6c5sc5ccccc56)cc4)cc3c3cc(-c4cccc5ccccc45)ccc32)cc1]

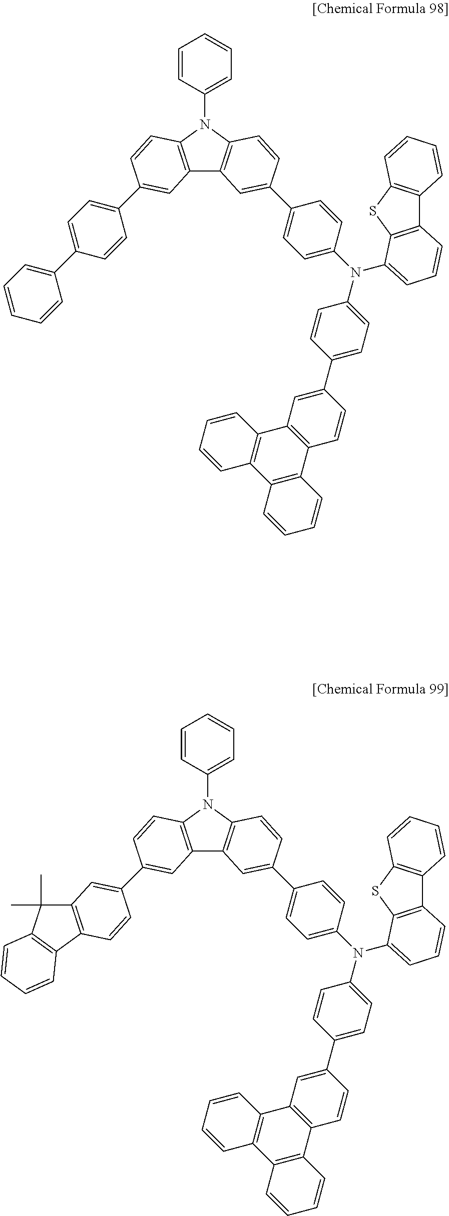 [SMILES: c1ccc(-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc21]

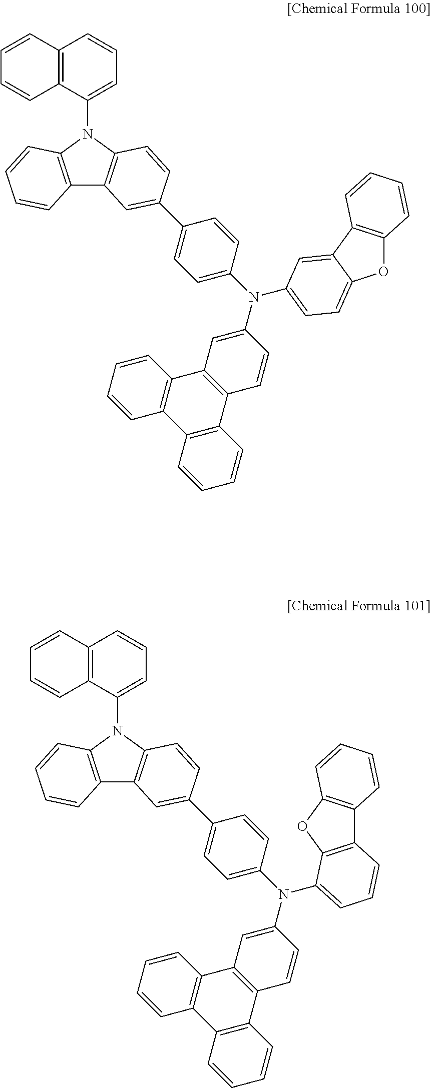 [SMILES: c1ccc2c(-n3c4ccccc4c4cc(-c5ccc(N(c6ccc7oc8ccccc8c7c6)c6ccc7c8ccccc8c8ccccc8c7c6)cc5)ccc43)cccc2c1 ; c1ccc2c(-n3c4ccccc4c4cc(-c5ccc(N(c6ccc7c8ccccc8c8ccccc8c7c6)c6cccc7c6oc6ccccc67)cc5)ccc43)cccc2c1]

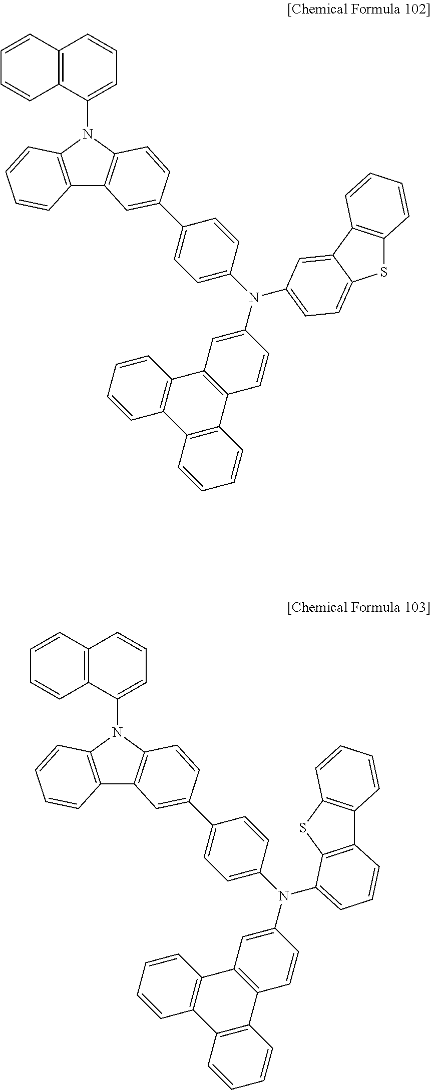 [SMILES: c1ccc2c(-n3c4ccccc4c4cc(-c5ccc(N(c6ccc7sc8ccccc8c7c6)c6ccc7c8ccccc8c8ccccc8c7c6)cc5)ccc43)cccc2c1 ; c1ccc2c(-n3c4ccccc4c4cc(-c5ccc(N(c6ccc7c8ccccc8c8ccccc8c7c6)c6cccc7c6sc6ccccc67)cc5)ccc43)cccc2c1]

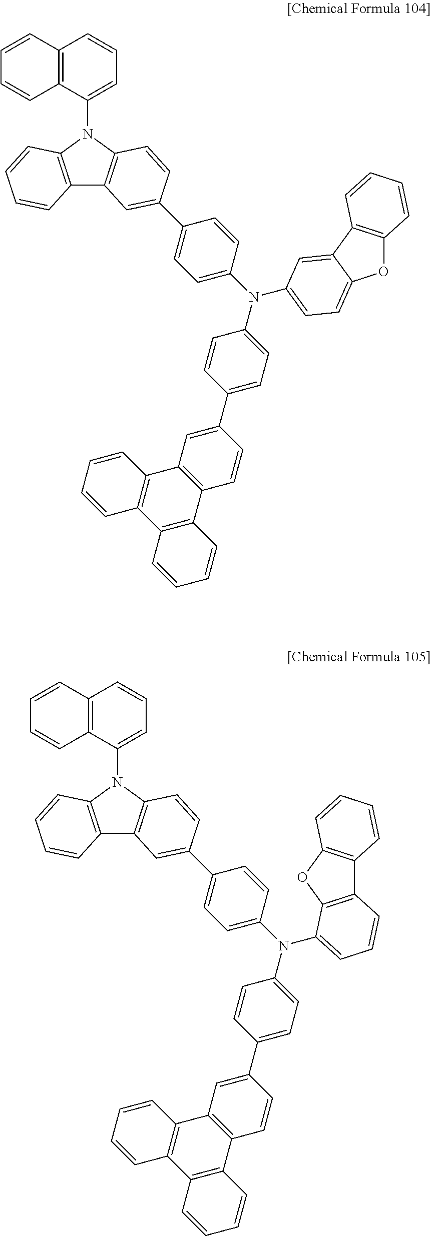 [SMILES: c1ccc2c(-n3c4ccccc4c4cc(-c5ccc(N(c6ccc(-c7ccc8c9ccccc9c9ccccc9c8c7)cc6)c6ccc7oc8ccccc8c7c6)cc5)ccc43)cccc2c1 ; c1ccc2c(-n3c4ccccc4c4cc(-c5ccc(N(c6ccc(-c7ccc8c9ccccc9c9ccccc9c8c7)cc6)c6cccc7c6oc6ccccc67)cc5)ccc43)cccc2c1]

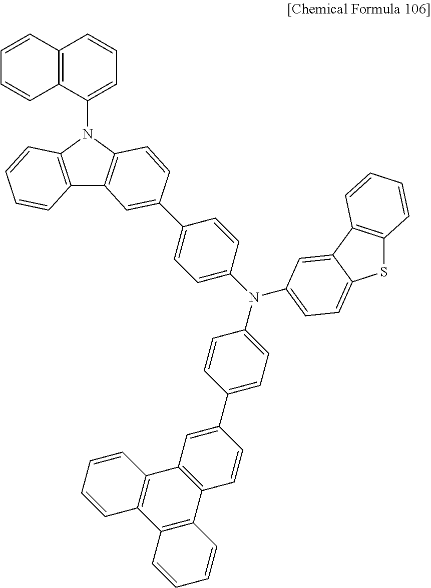 [SMILES: c1ccc2c(-n3c4ccccc4c4cc(-c5ccc(N(c6ccc(-c7ccc8c9ccccc9c9ccccc9c8c7)cc6)c6ccc7sc8ccccc8c7c6)cc5)ccc43)cccc2c1]

The compound represented by the above Chemical Formula 3 may be represented by, e.g., one of the following Chemical Formulae ad3 to ad5.

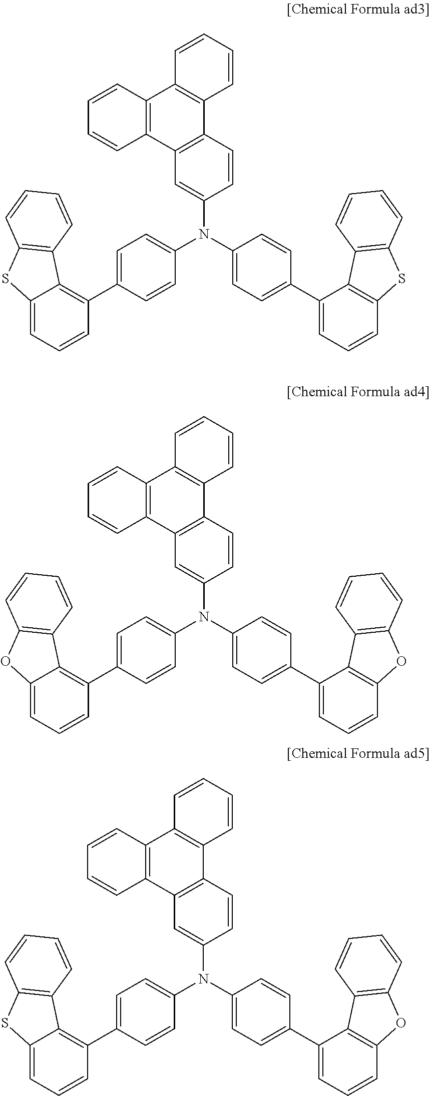 [SMILES: c1ccc2c(c1)sc1cccc(-c3ccc(N(c4ccc(-c5cccc6sc7ccccc7c56)cc4)c4ccc5c6ccccc6c6ccccc6c5c4)cc3)c12 ; c1ccc2c(c1)oc1cccc(-c3ccc(N(c4ccc(-c5cccc6oc7ccccc7c56)cc4)c4ccc5c6ccccc6c6ccccc6c5c4)cc3)c12 ; c1ccc2c(c1)oc1cccc(-c3ccc(N(c4ccc(-c5cccc6sc7ccccc7c56)cc4)c4ccc5c6ccccc6c6ccccc6c5c4)cc3)c12]

The compound represented by the above Chemical Formula 2 may be represented by, e.g., one of the following Chemical Formulae 107 to 333.

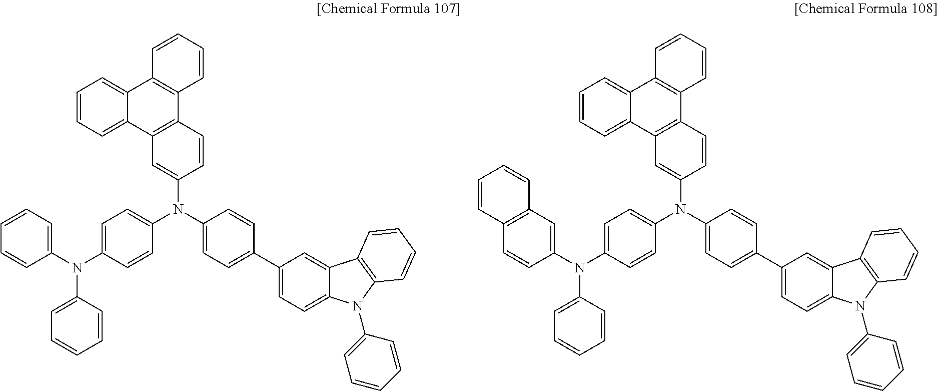 [SMILES: c1ccc(N(c2ccccc2)c2ccc(N(c3ccc(-c4ccc5c(c4)c4ccccc4n5-c4ccccc4)cc3)c3ccc4c5ccccc5c5ccccc5c4c3)cc2)cc1 ; c1ccc(N(c2ccc(N(c3ccc(-c4ccc5c(c4)c4ccccc4n5-c4ccccc4)cc3)c3ccc4c5ccccc5c5ccccc5c4c3)cc2)c2ccc3ccccc3c2)cc1]

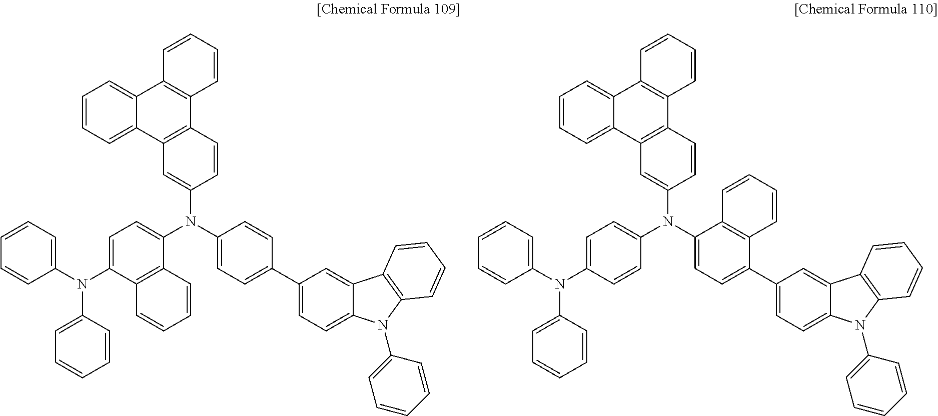

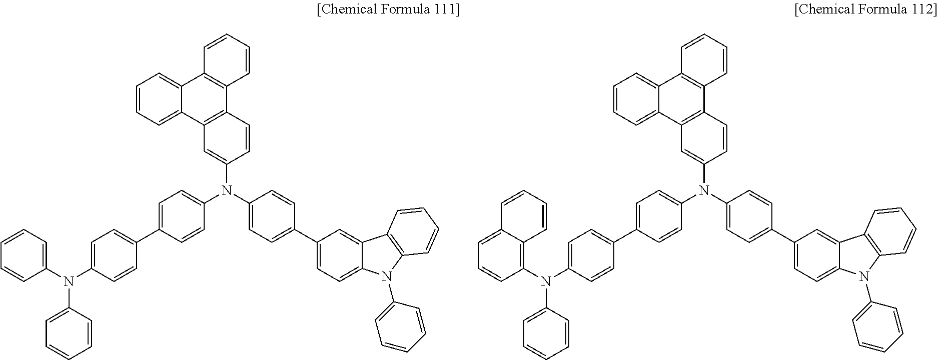 [SMILES: c1ccc(N(c2ccccc2)c2ccc(-c3ccc(N(c4ccc(-c5ccc6c(c5)c5ccccc5n6-c5ccccc5)cc4)c4ccc5c6ccccc6c6ccccc6c5c4)cc3)cc2)cc1 ; c1ccc(N(c2ccc(-c3ccc(N(c4ccc(-c5ccc6c(c5)c5ccccc5n6-c5ccccc5)cc4)c4ccc5c6ccccc6c6ccccc6c5c4)cc3)cc2)c2cccc3ccccc23)cc1]

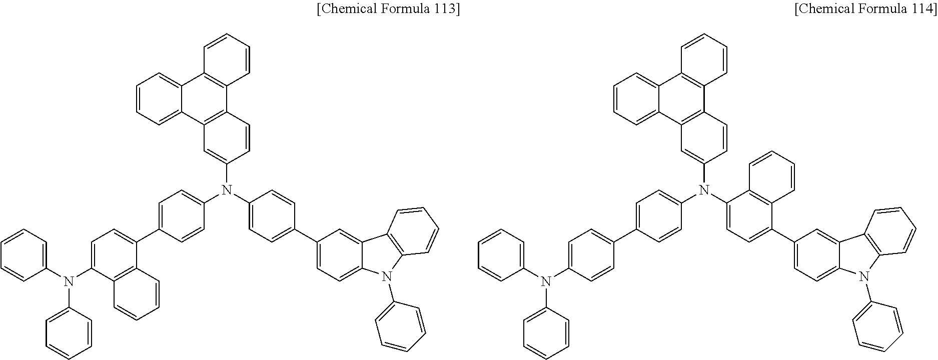 [SMILES: c1ccc(N(c2ccccc2)c2ccc(-c3ccc(N(c4ccc(-c5ccc6c(c5)c5ccccc5n6-c5ccccc5)cc4)c4ccc5c6ccccc6c6ccccc6c5c4)cc3)c3ccccc23)cc1 ; c1ccc(N(c2ccccc2)c2ccc(-c3ccc(N(c4ccc5c6ccccc6c6ccccc6c5c4)c4ccc(-c5ccc6c(c5)c5ccccc5n6-c5ccccc5)c5ccccc45)cc3)cc2)cc1]

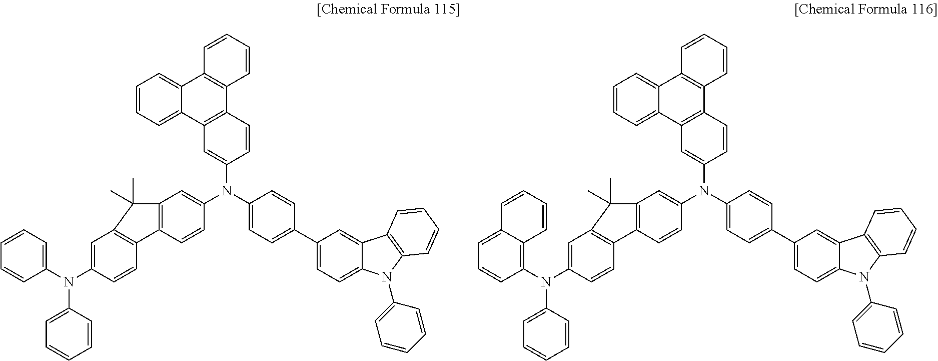 [SMILES: CC1(C)c2cc(N(c3ccccc3)c3ccccc3)ccc2-c2ccc(N(c3ccc(-c4ccc5c(c4)c4ccccc4n5-c4ccccc4)cc3)c3ccc4c5ccccc5c5ccccc5c4c3)cc21 ; CC1(C)c2cc(N(c3ccc(-c4ccc5c(c4)c4ccccc4n5-c4ccccc4)cc3)c3ccc4c5ccccc5c5ccccc5c4c3)ccc2-c2ccc(N(c3ccccc3)c3cccc4ccccc34)cc21]

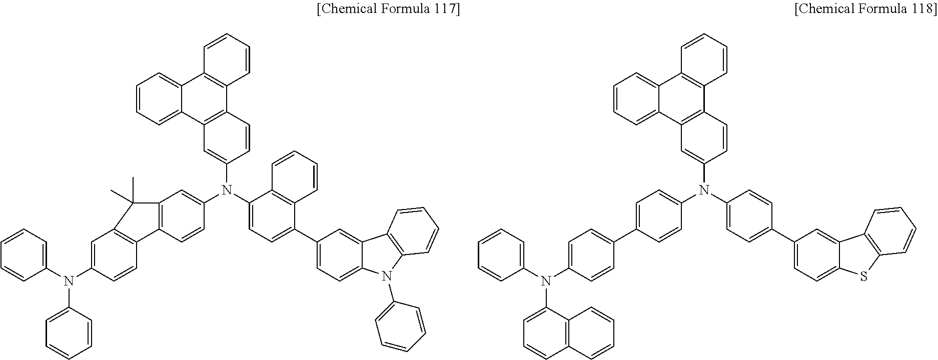 [SMILES: CC1(C)c2cc(N(c3ccccc3)c3ccccc3)ccc2-c2ccc(N(c3ccc4c5ccccc5c5ccccc5c4c3)c3ccc(-c4ccc5c(c4)c4ccccc4n5-c4ccccc4)c4ccccc34)cc21 ; c1ccc(N(c2ccc(-c3ccc(N(c4ccc(-c5ccc6sc7ccccc7c6c5)cc4)c4ccc5c6ccccc6c6ccccc6c5c4)cc3)cc2)c2cccc3ccccc23)cc1]

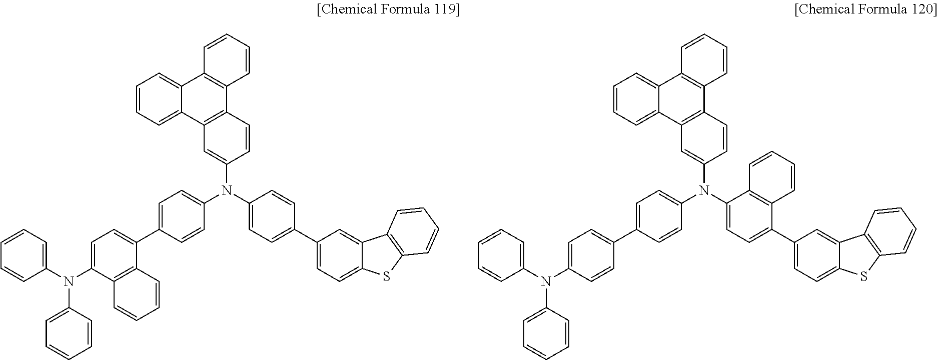 [SMILES: c1ccc(N(c2ccccc2)c2ccc(-c3ccc(N(c4ccc(-c5ccc6sc7ccccc7c6c5)cc4)c4ccc5c6ccccc6c6ccccc6c5c4)cc3)c3ccccc23)cc1 ; c1ccc(N(c2ccccc2)c2ccc(-c3ccc(N(c4ccc5c6ccccc6c6ccccc6c5c4)c4ccc(-c5ccc6sc7ccccc7c6c5)c5ccccc45)cc3)cc2)cc1]

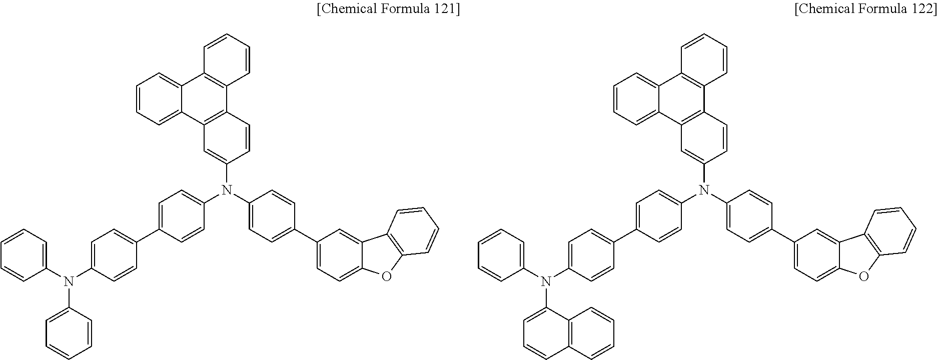 [SMILES: c1ccc(N(c2ccccc2)c2ccc(-c3ccc(N(c4ccc(-c5ccc6oc7ccccc7c6c5)cc4)c4ccc5c6ccccc6c6ccccc6c5c4)cc3)cc2)cc1 ; c1ccc(N(c2ccc(-c3ccc(N(c4ccc(-c5ccc6oc7ccccc7c6c5)cc4)c4ccc5c6ccccc6c6ccccc6c5c4)cc3)cc2)c2cccc3ccccc23)cc1]

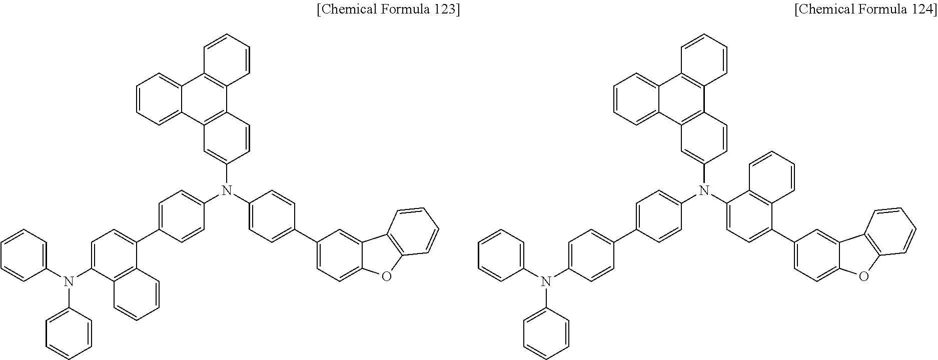 [SMILES: c1ccc(N(c2ccccc2)c2ccc(-c3ccc(N(c4ccc(-c5ccc6oc7ccccc7c6c5)cc4)c4ccc5c6ccccc6c6ccccc6c5c4)cc3)c3ccccc23)cc1 ; c1ccc(N(c2ccccc2)c2ccc(-c3ccc(N(c4ccc5c6ccccc6c6ccccc6c5c4)c4ccc(-c5ccc6oc7ccccc7c6c5)c5ccccc45)cc3)cc2)cc1]

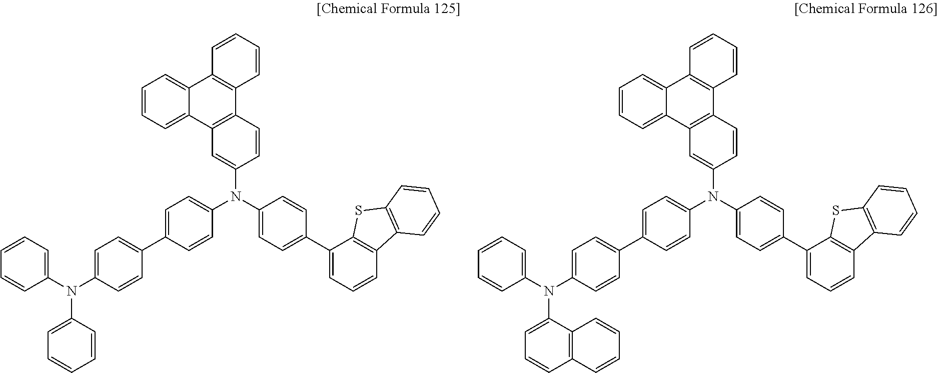 [SMILES: c1ccc(N(c2ccccc2)c2ccc(-c3ccc(N(c4ccc(-c5cccc6c5sc5ccccc56)cc4)c4ccc5c6ccccc6c6ccccc6c5c4)cc3)cc2)cc1 ; c1ccc(N(c2ccc(-c3ccc(N(c4ccc(-c5cccc6c5sc5ccccc56)cc4)c4ccc5c6ccccc6c6ccccc6c5c4)cc3)cc2)c2cccc3ccccc23)cc1]

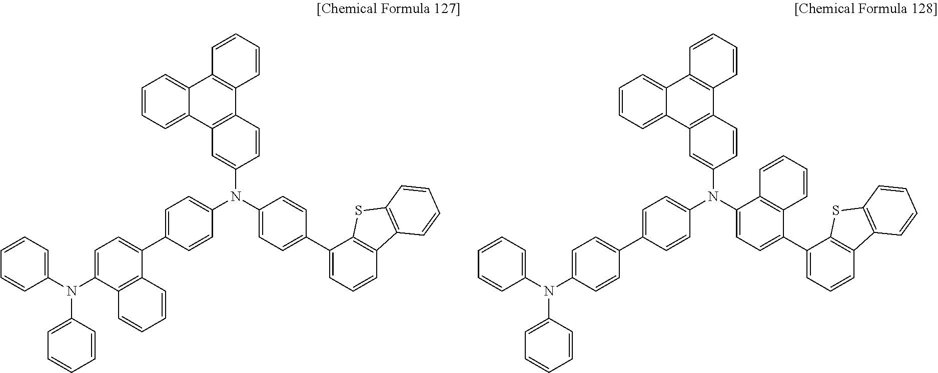 [SMILES: c1ccc(N(c2ccccc2)c2ccc(-c3ccc(N(c4ccc(-c5cccc6c5sc5ccccc56)cc4)c4ccc5c6ccccc6c6ccccc6c5c4)cc3)c3ccccc23)cc1 ; c1ccc(N(c2ccccc2)c2ccc(-c3ccc(N(c4ccc5c6ccccc6c6ccccc6c5c4)c4ccc(-c5cccc6c5sc5ccccc56)c5ccccc45)cc3)cc2)cc1]

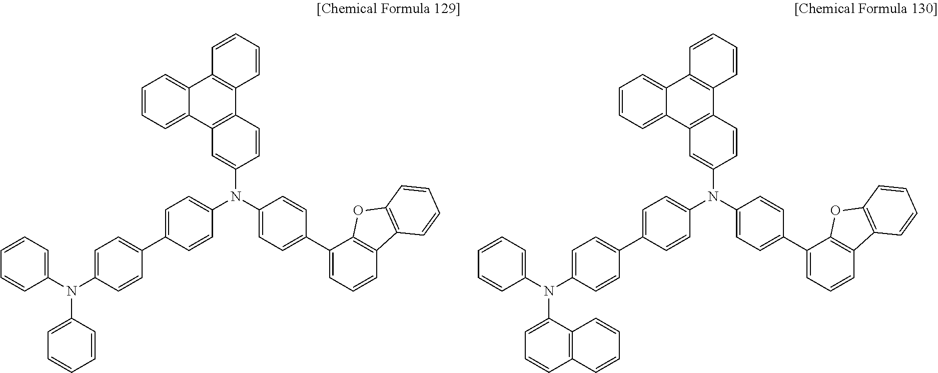 [SMILES: c1ccc(N(c2ccccc2)c2ccc(-c3ccc(N(c4ccc(-c5cccc6c5oc5ccccc56)cc4)c4ccc5c6ccccc6c6ccccc6c5c4)cc3)cc2)cc1 ; c1ccc(N(c2ccc(-c3ccc(N(c4ccc(-c5cccc6c5oc5ccccc56)cc4)c4ccc5c6ccccc6c6ccccc6c5c4)cc3)cc2)c2cccc3ccccc23)cc1]

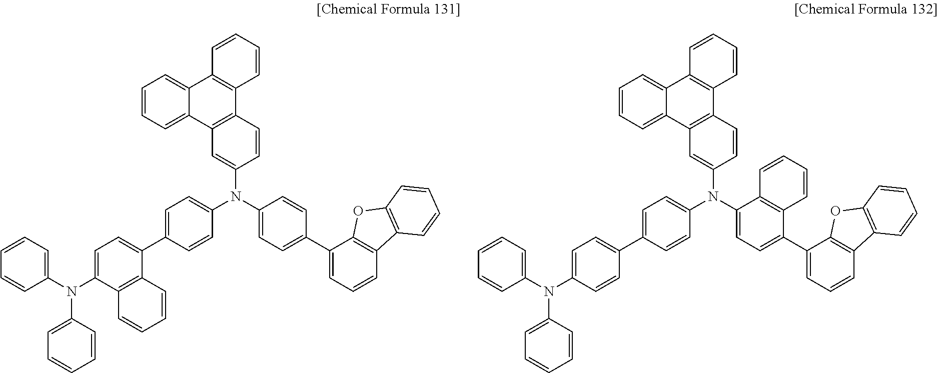 [SMILES: c1ccc(N(c2ccccc2)c2ccc(-c3ccc(N(c4ccc(-c5cccc6c5oc5ccccc56)cc4)c4ccc5c6ccccc6c6ccccc6c5c4)cc3)c3ccccc23)cc1 ; c1ccc(N(c2ccccc2)c2ccc(-c3ccc(N(c4ccc5c6ccccc6c6ccccc6c5c4)c4ccc(-c5cccc6c5oc5ccccc56)c5ccccc45)cc3)cc2)cc1]

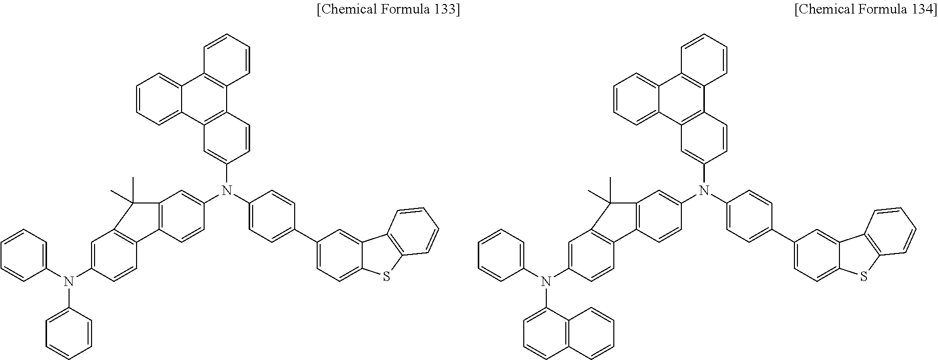 [SMILES: CC1(C)c2cc(N(c3ccccc3)c3ccccc3)ccc2-c2ccc(N(c3ccc(-c4ccc5sc6ccccc6c5c4)cc3)c3ccc4c5ccccc5c5ccccc5c4c3)cc21 ; CC1(C)c2cc(N(c3ccc(-c4ccc5sc6ccccc6c5c4)cc3)c3ccc4c5ccccc5c5ccccc5c4c3)ccc2-c2ccc(N(c3ccccc3)c3cccc4ccccc34)cc21]

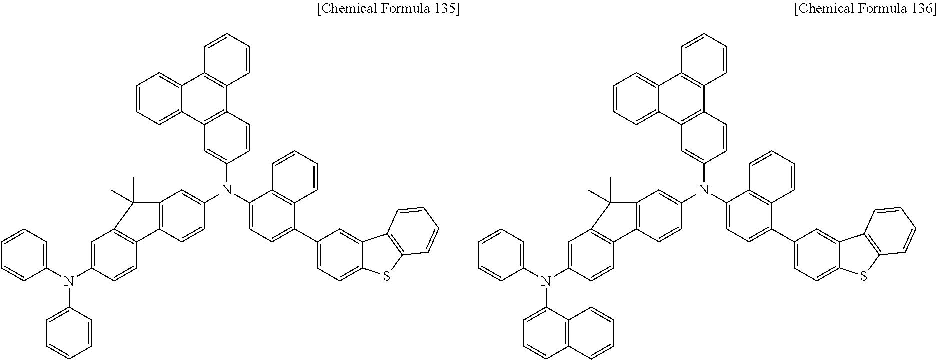 [SMILES: CC1(C)c2cc(N(c3ccccc3)c3ccccc3)ccc2-c2ccc(N(c3ccc4c5ccccc5c5ccccc5c4c3)c3ccc(-c4ccc5sc6ccccc6c5c4)c4ccccc34)cc21 ; CC1(C)c2cc(N(c3ccccc3)c3cccc4ccccc34)ccc2-c2ccc(N(c3ccc4c5ccccc5c5ccccc5c4c3)c3ccc(-c4ccc5sc6ccccc6c5c4)c4ccccc34)cc21]

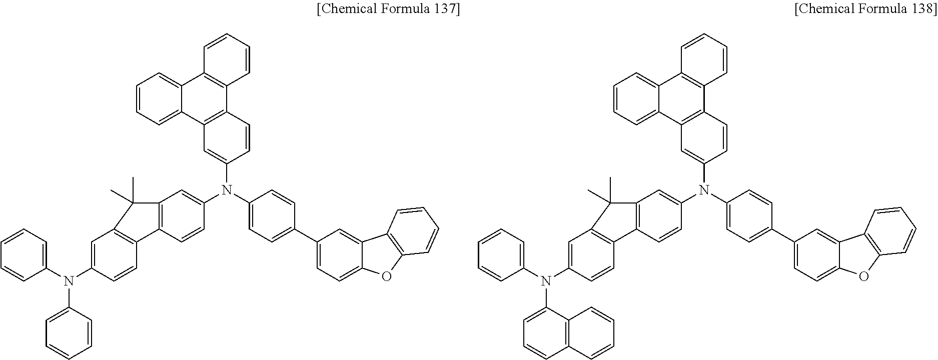 [SMILES: CC1(C)c2cc(N(c3ccccc3)c3ccccc3)ccc2-c2ccc(N(c3ccc(-c4ccc5oc6ccccc6c5c4)cc3)c3ccc4c5ccccc5c5ccccc5c4c3)cc21 ; CC1(C)c2cc(N(c3ccc(-c4ccc5oc6ccccc6c5c4)cc3)c3ccc4c5ccccc5c5ccccc5c4c3)ccc2-c2ccc(N(c3ccccc3)c3cccc4ccccc34)cc21]

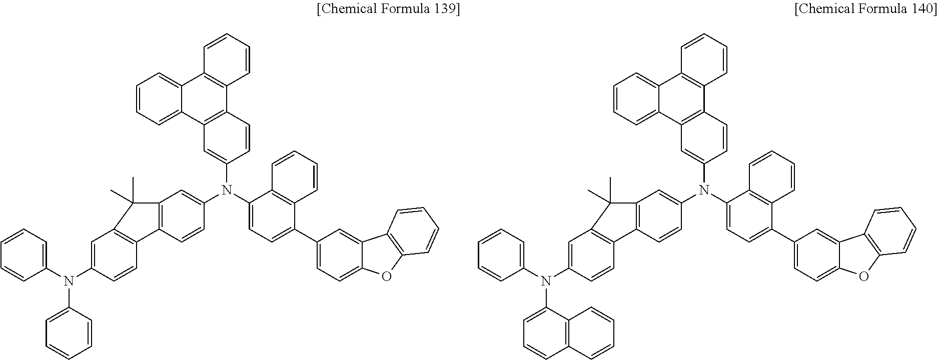 [SMILES: CC1(C)c2cc(N(c3ccccc3)c3ccccc3)ccc2-c2ccc(N(c3ccc4c5ccccc5c5ccccc5c4c3)c3ccc(-c4ccc5oc6ccccc6c5c4)c4ccccc34)cc21 ; CC1(C)c2cc(N(c3ccccc3)c3cccc4ccccc34)ccc2-c2ccc(N(c3ccc4c5ccccc5c5ccccc5c4c3)c3ccc(-c4ccc5oc6ccccc6c5c4)c4ccccc34)cc21]

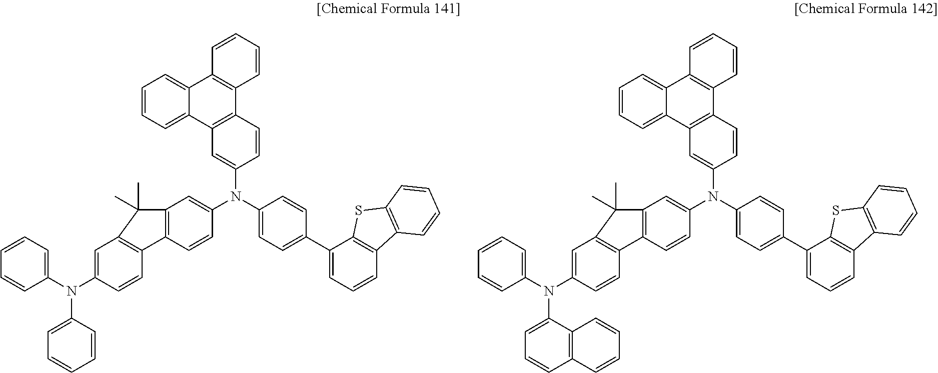 [SMILES: CC1(C)c2cc(N(c3ccccc3)c3ccccc3)ccc2-c2ccc(N(c3ccc(-c4cccc5c4sc4ccccc45)cc3)c3ccc4c5ccccc5c5ccccc5c4c3)cc21 ; CC1(C)c2cc(N(c3ccc(-c4cccc5c4sc4ccccc45)cc3)c3ccc4c5ccccc5c5ccccc5c4c3)ccc2-c2ccc(N(c3ccccc3)c3cccc4ccccc34)cc21]

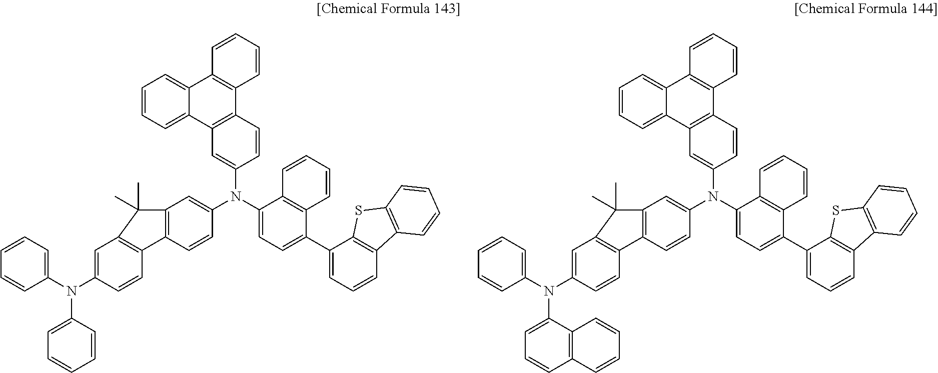 [SMILES: CC1(C)c2cc(N(c3ccccc3)c3ccccc3)ccc2-c2ccc(N(c3ccc4c5ccccc5c5ccccc5c4c3)c3ccc(-c4cccc5c4sc4ccccc45)c4ccccc34)cc21 ; CC1(C)c2cc(N(c3ccccc3)c3cccc4ccccc34)ccc2-c2ccc(N(c3ccc4c5ccccc5c5ccccc5c4c3)c3ccc(-c4cccc5c4sc4ccccc45)c4ccccc34)cc21]

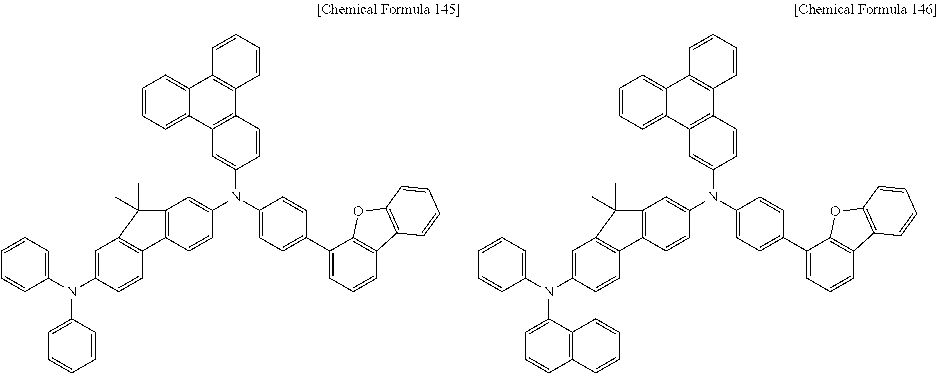 [SMILES: CC1(C)c2cc(N(c3ccccc3)c3ccccc3)ccc2-c2ccc(N(c3ccc(-c4cccc5c4oc4ccccc45)cc3)c3ccc4c5ccccc5c5ccccc5c4c3)cc21 ; CC1(C)c2cc(N(c3ccc(-c4cccc5c4oc4ccccc45)cc3)c3ccc4c5ccccc5c5ccccc5c4c3)ccc2-c2ccc(N(c3ccccc3)c3cccc4ccccc34)cc21]

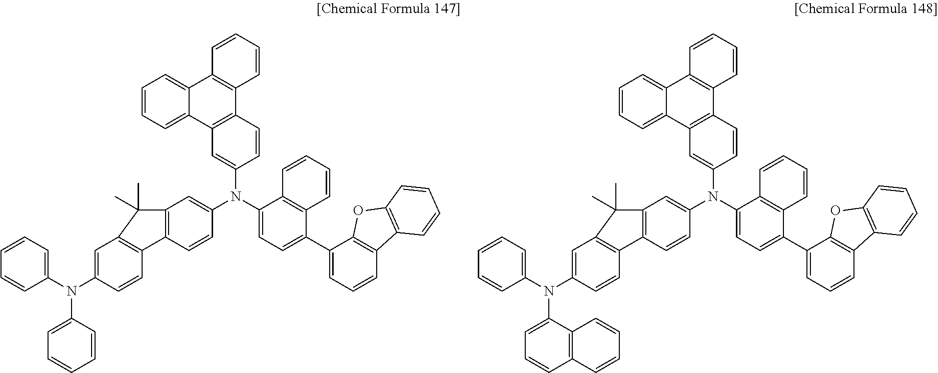 [SMILES: CC1(C)c2cc(N(c3ccccc3)c3ccccc3)ccc2-c2ccc(N(c3ccc4c5ccccc5c5ccccc5c4c3)c3ccc(-c4cccc5c4oc4ccccc45)c4ccccc34)cc21 ; CC1(C)c2cc(N(c3ccccc3)c3cccc4ccccc34)ccc2-c2ccc(N(c3ccc4c5ccccc5c5ccccc5c4c3)c3ccc(-c4cccc5c4oc4ccccc45)c4ccccc34)cc21]

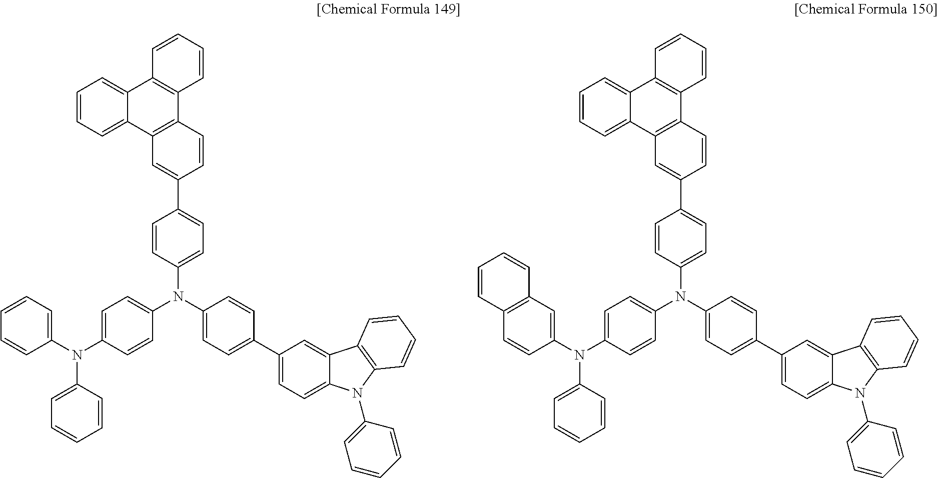 [SMILES: c1ccc(N(c2ccccc2)c2ccc(N(c3ccc(-c4ccc5c6ccccc6c6ccccc6c5c4)cc3)c3ccc(-c4ccc5c(c4)c4ccccc4n5-c4ccccc4)cc3)cc2)cc1 ; c1ccc(N(c2ccc(N(c3ccc(-c4ccc5c6ccccc6c6ccccc6c5c4)cc3)c3ccc(-c4ccc5c(c4)c4ccccc4n5-c4ccccc4)cc3)cc2)c2ccc3ccccc3c2)cc1]

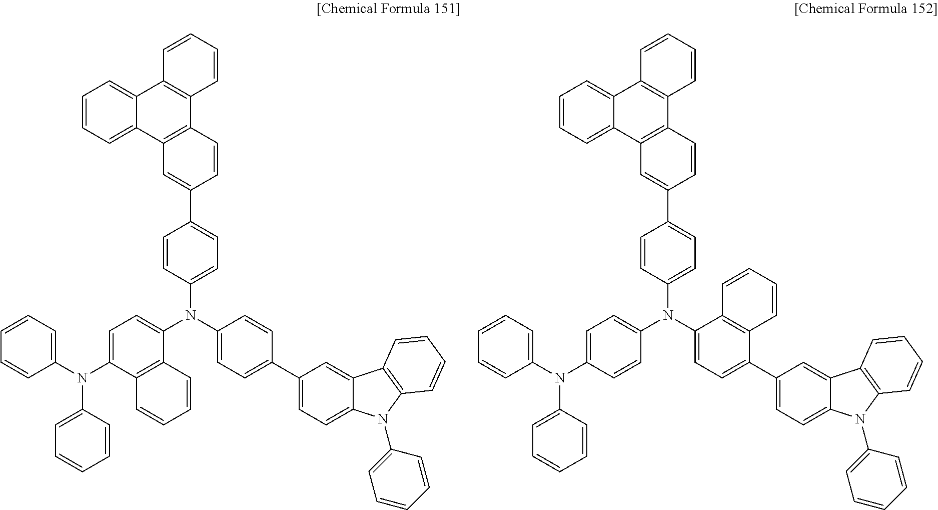

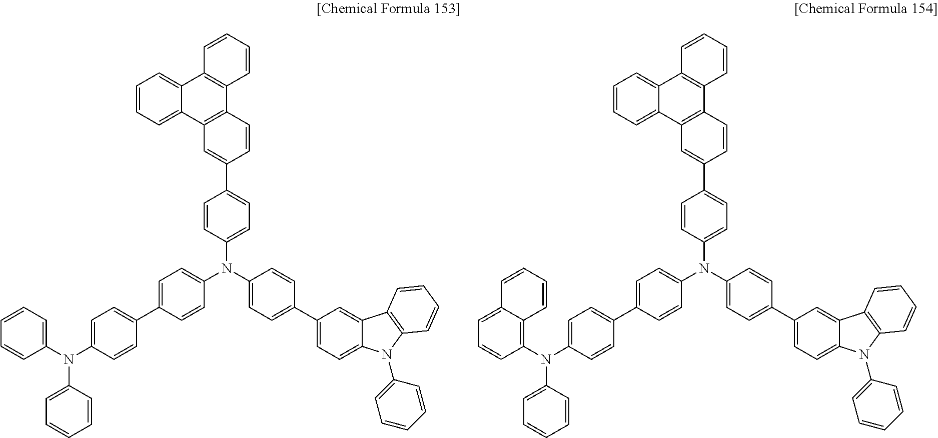 [SMILES: c1ccc(N(c2ccccc2)c2ccc(-c3ccc(N(c4ccc(-c5ccc6c7ccccc7c7ccccc7c6c5)cc4)c4ccc(-c5ccc6c(c5)c5ccccc5n6-c5ccccc5)cc4)cc3)cc2)cc1 ; c1ccc(N(c2ccc(-c3ccc(N(c4ccc(-c5ccc6c7ccccc7c7ccccc7c6c5)cc4)c4ccc(-c5ccc6c(c5)c5ccccc5n6-c5ccccc5)cc4)cc3)cc2)c2cccc3ccccc23)cc1]

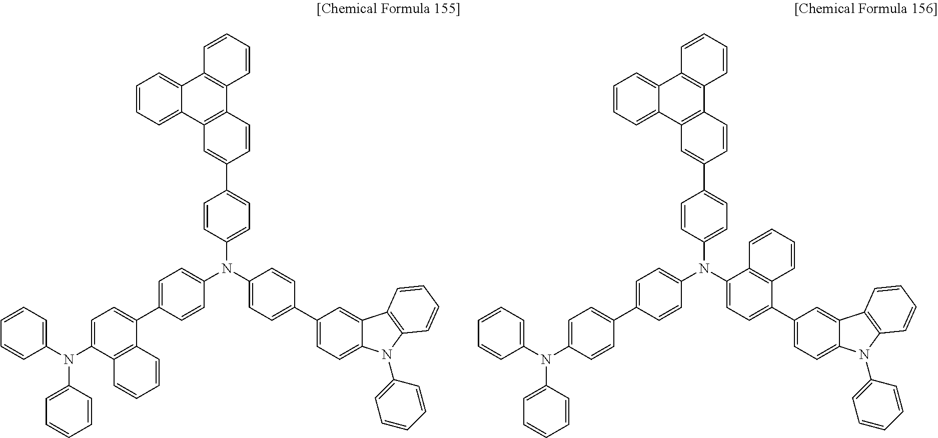 [SMILES: c1ccc(N(c2ccccc2)c2ccc(-c3ccc(N(c4ccc(-c5ccc6c7ccccc7c7ccccc7c6c5)cc4)c4ccc(-c5ccc6c(c5)c5ccccc5n6-c5ccccc5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc6c(c5)c5ccccc5n6-c5ccccc5)c5ccccc45)cc3)cc2)cc1]

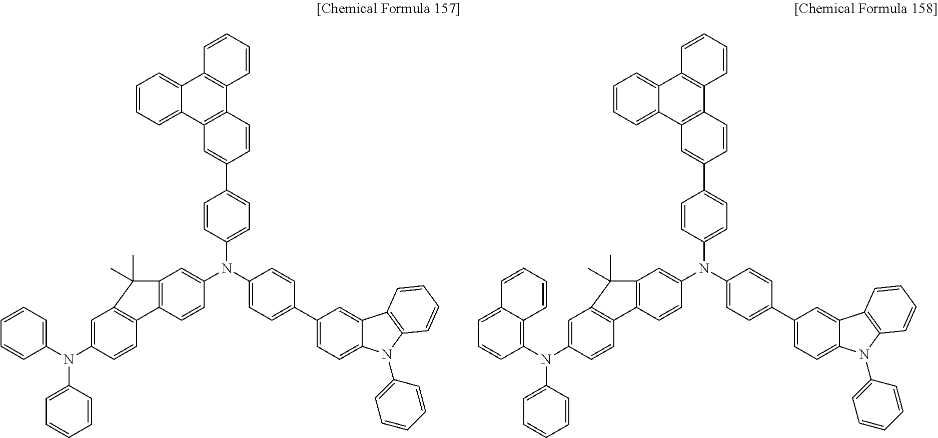 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc5c(c4)c4ccccc4n5-c4ccccc4)cc3)cc21 ; CC1(C)c2cc(N(c3ccc(-c4ccc5c6ccccc6c6ccccc6c5c4)cc3)c3ccc(-c4ccc5c(c4)c4ccccc4n5-c4ccccc4)cc3)ccc2-c2ccc(N(c3ccccc3)c3cccc4ccccc34)cc21]

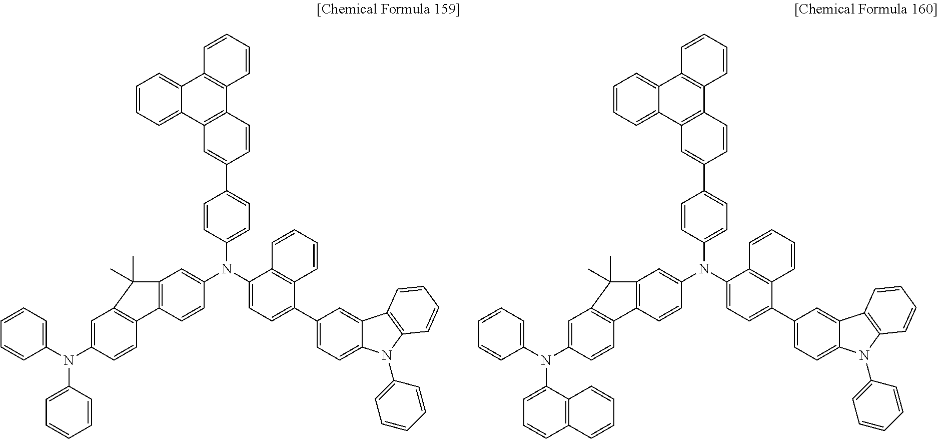 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc5c(c4)c4ccccc4n5-c4ccccc4)c4ccccc34)cc21 ; CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4ccc5c(c4)c4ccccc4n5-c4ccccc4)c4ccccc34)cc21]

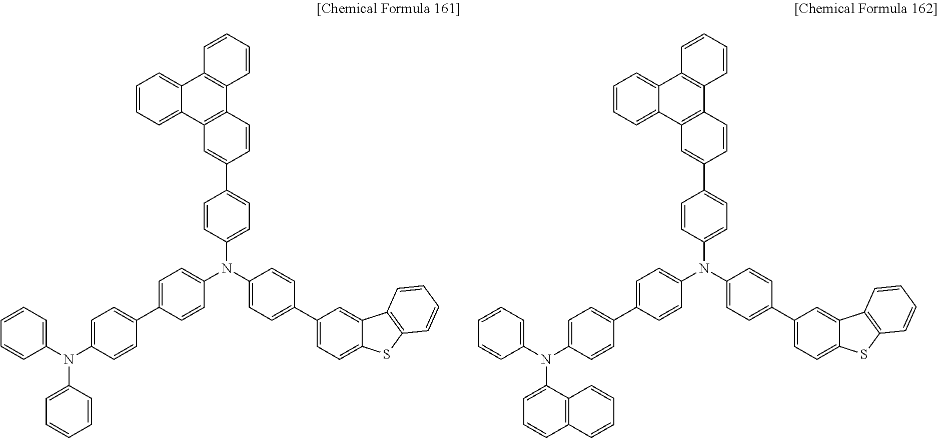 [SMILES: c1ccc(N(c2ccccc2)c2ccc(-c3ccc(N(c4ccc(-c5ccc6sc7ccccc7c6c5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc6sc7ccccc7c6c5)cc4)c4ccc(-c5ccc6c7ccccc7c7ccccc7c6c5)cc4)cc3)cc2)c2cccc3ccccc23)cc1]

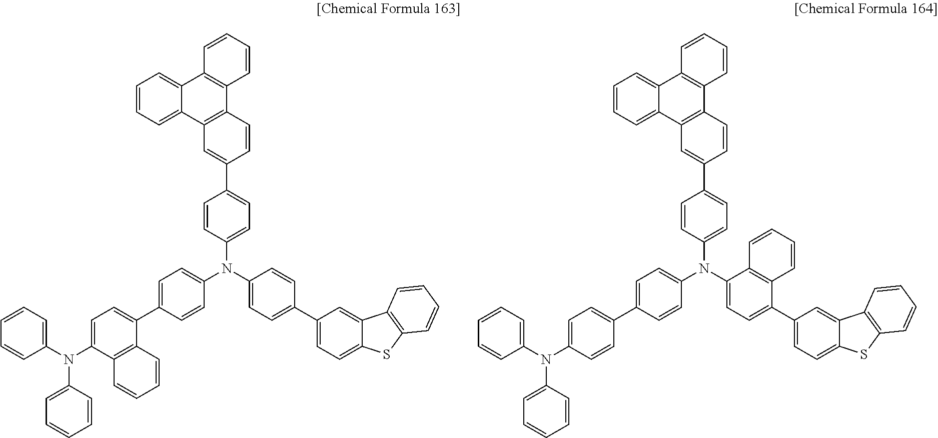 [SMILES: c1ccc(N(c2ccccc2)c2ccc(-c3ccc(N(c4ccc(-c5ccc6sc7ccccc7c6c5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc6sc7ccccc7c6c5)c5ccccc45)cc3)cc2)cc1]

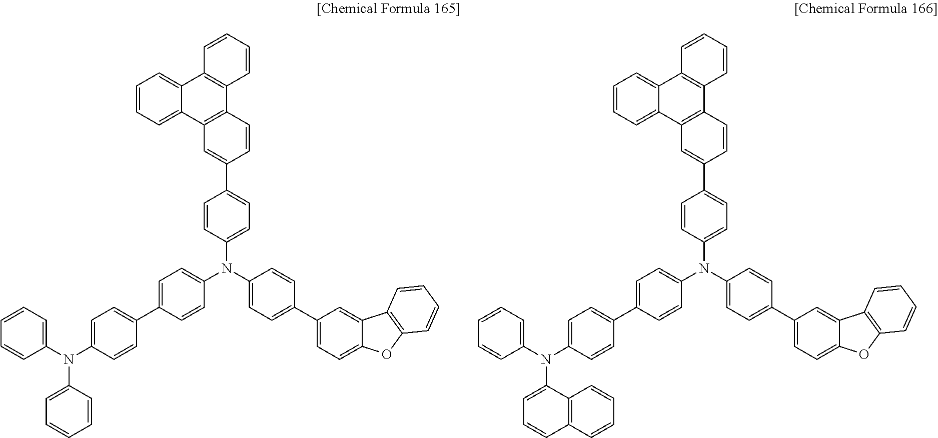 [SMILES: c1ccc(N(c2ccccc2)c2ccc(-c3ccc(N(c4ccc(-c5ccc6oc7ccccc7c6c5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc6oc7ccccc7c6c5)cc4)c4ccc(-c5ccc6c7ccccc7c7ccccc7c6c5)cc4)cc3)cc2)c2cccc3ccccc23)cc1]

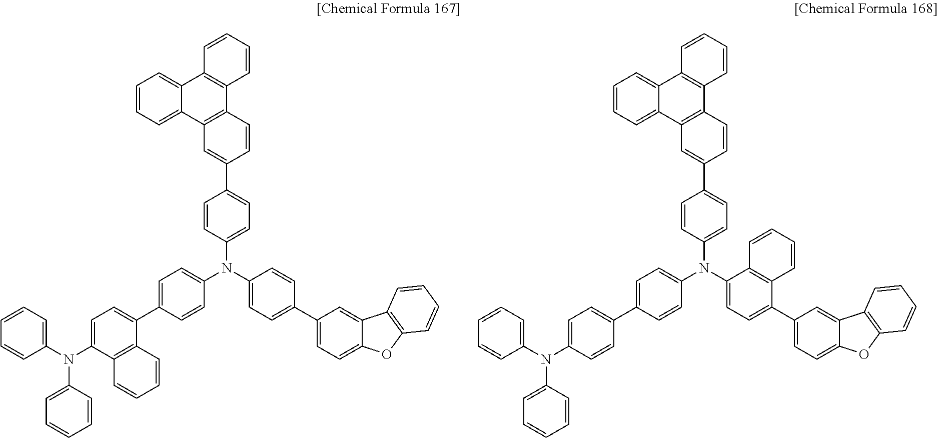 [SMILES: c1ccc(N(c2ccccc2)c2ccc(-c3ccc(N(c4ccc(-c5ccc6oc7ccccc7c6c5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc6oc7ccccc7c6c5)c5ccccc45)cc3)cc2)cc1]

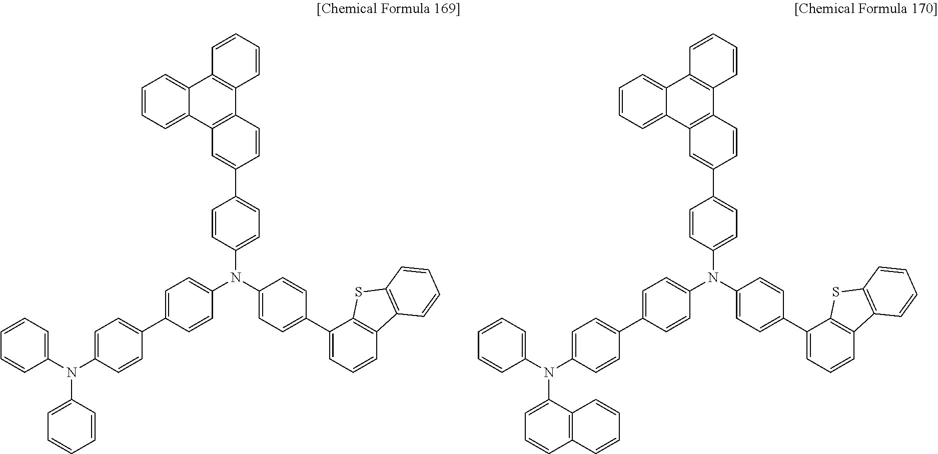 [SMILES: c1ccc(N(c2ccccc2)c2ccc(-c3ccc(N(c4ccc(-c5ccc6c7ccccc7c7ccccc7c6c5)cc4)c4ccc(-c5cccc6c5sc5ccccc56)cc4)cc3)cc2)cc1 ; c1ccc(N(c2ccc(-c3ccc(N(c4ccc(-c5ccc6c7ccccc7c7ccccc7c6c5)cc4)c4ccc(-c5cccc6c5sc5ccccc56)cc4)cc3)cc2)c2cccc3ccccc23)cc1]

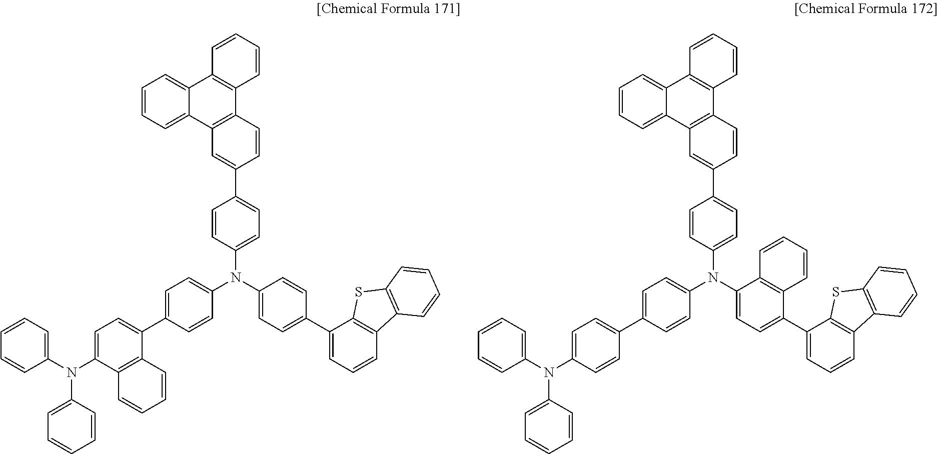 [SMILES: c1ccc(N(c2ccccc2)c2ccc(-c3ccc(N(c4ccc(-c5ccc6c7ccccc7c7ccccc7c6c5)cc4)c4ccc(-c5cccc6c5sc5ccccc56)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5cccc6c5sc5ccccc56)c5ccccc45)cc3)cc2)cc1]

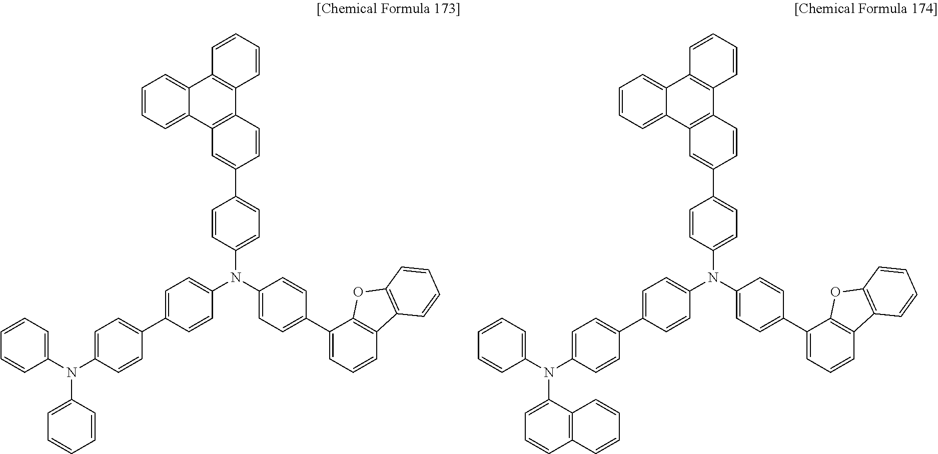 [SMILES: c1ccc(N(c2ccccc2)c2ccc(-c3ccc(N(c4ccc(-c5ccc6c7ccccc7c7ccccc7c6c5)cc4)c4ccc(-c5cccc6c5oc5ccccc56)cc4)cc3)cc2)cc1 ; c1ccc(N(c2ccc(-c3ccc(N(c4ccc(-c5ccc6c7ccccc7c7ccccc7c6c5)cc4)c4ccc(-c5cccc6c5oc5ccccc56)cc4)cc3)cc2)c2cccc3ccccc23)cc1]

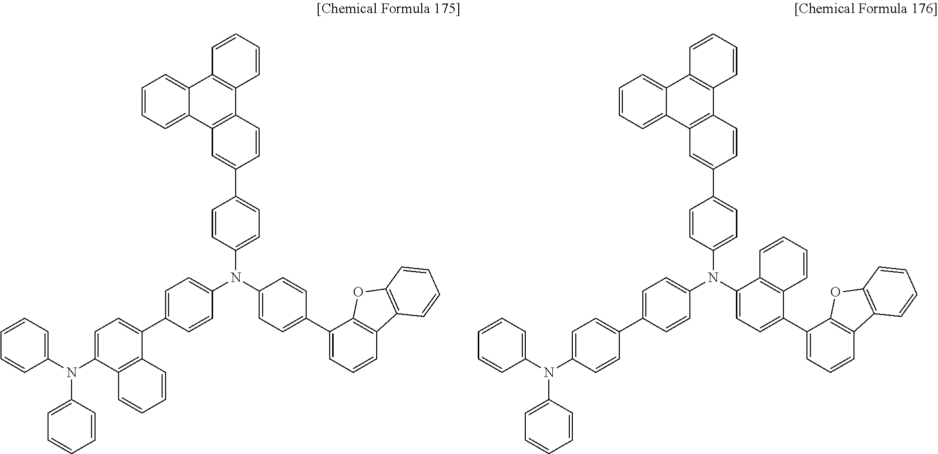 [SMILES: c1ccc(N(c2ccccc2)c2ccc(-c3ccc(N(c4ccc(-c5ccc6c7ccccc7c7ccccc7c6c5)cc4)c4ccc(-c5cccc6c5oc5ccccc56)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5cccc6c5oc5ccccc56)c5ccccc45)cc3)cc2)cc1]

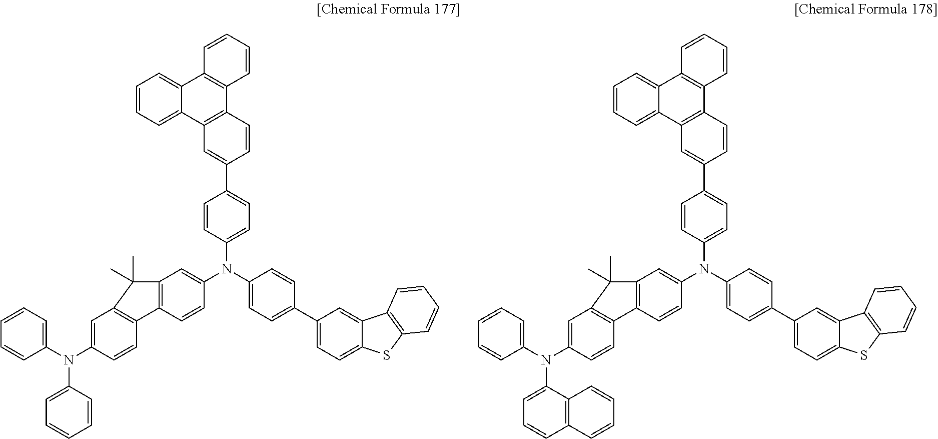 [SMILES: CC1(C)c2cc(N(c3ccccc3)c3ccccc3)ccc2-c2ccc(N(c3ccc(-c4ccc5sc6ccccc6c5c4)cc3)c3ccc(-c4ccc5c6ccccc6c6ccccc6c5c4)cc3)cc21 ; CC1(C)c2cc(N(c3ccc(-c4ccc5sc6ccccc6c5c4)cc3)c3ccc(-c4ccc5c6ccccc6c6ccccc6c5c4)cc3)ccc2-c2ccc(N(c3ccccc3)c3cccc4ccccc34)cc21]

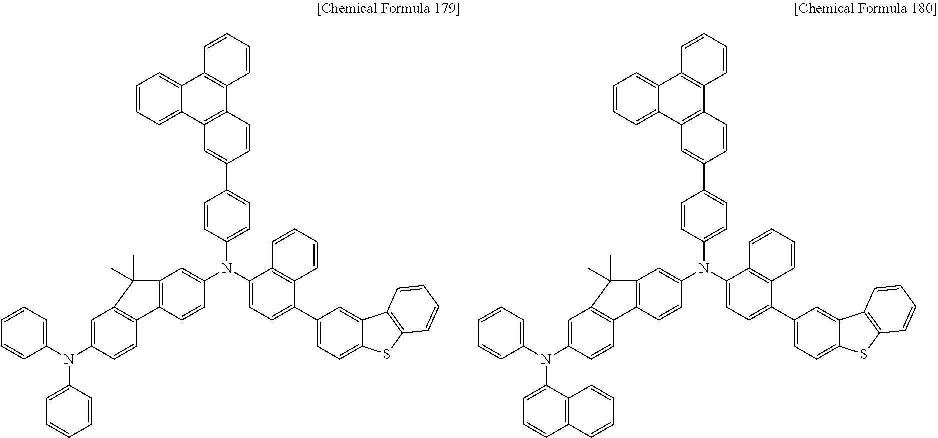 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc5sc6ccccc6c5c4)c4ccccc34)cc21 ; CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4ccc5sc6ccccc6c5c4)c4ccccc34)cc21]

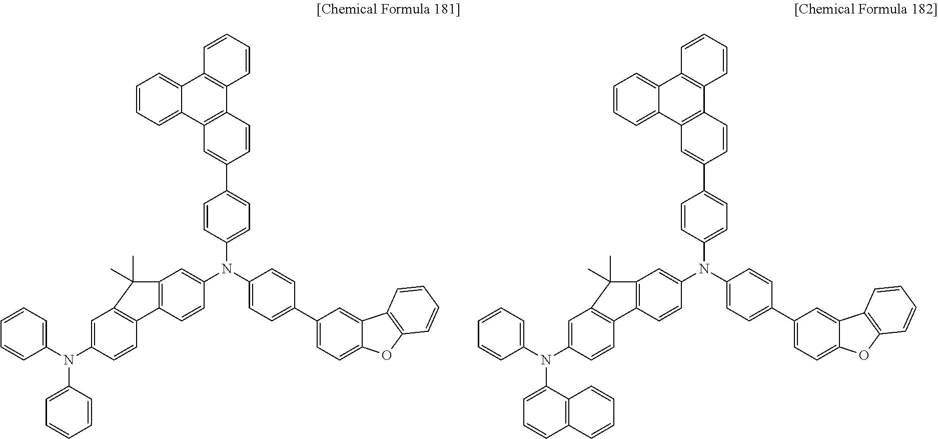 [SMILES: CC1(C)c2cc(N(c3ccccc3)c3ccccc3)ccc2-c2ccc(N(c3ccc(-c4ccc5oc6ccccc6c5c4)cc3)c3ccc(-c4ccc5c6ccccc6c6ccccc6c5c4)cc3)cc21 ; CC1(C)c2cc(N(c3ccc(-c4ccc5oc6ccccc6c5c4)cc3)c3ccc(-c4ccc5c6ccccc6c6ccccc6c5c4)cc3)ccc2-c2ccc(N(c3ccccc3)c3cccc4ccccc34)cc21]

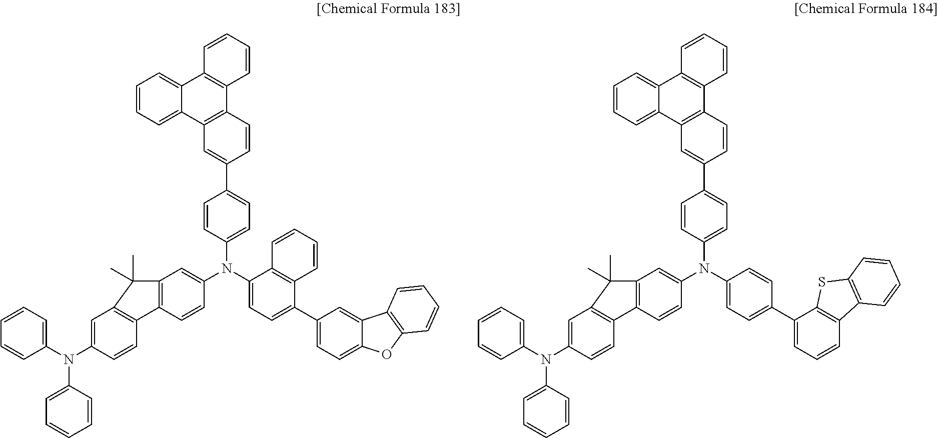 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc5oc6ccccc6c5c4)c4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4cccc5c4sc4ccccc45)cc3)cc21]

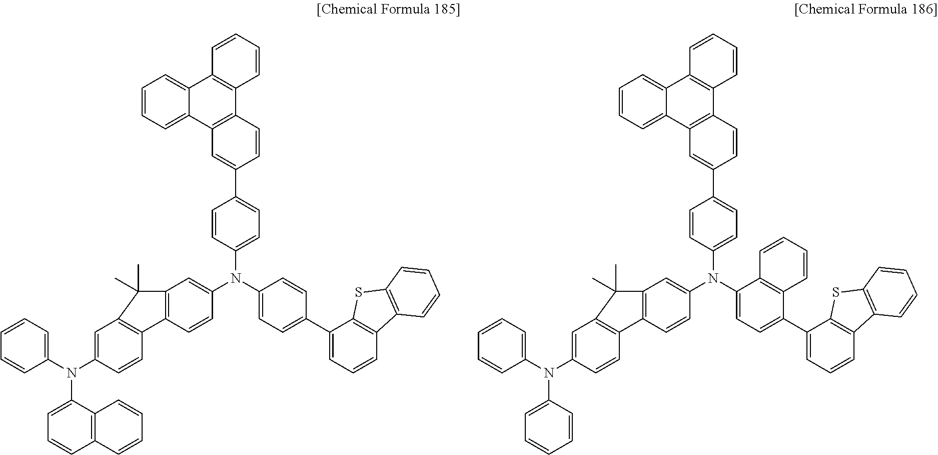 [SMILES: CC1(C)c2cc(N(c3ccc(-c4ccc5c6ccccc6c6ccccc6c5c4)cc3)c3ccc(-c4cccc5c4sc4ccccc45)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4cccc5c4sc4ccccc45)c4ccccc34)cc21]

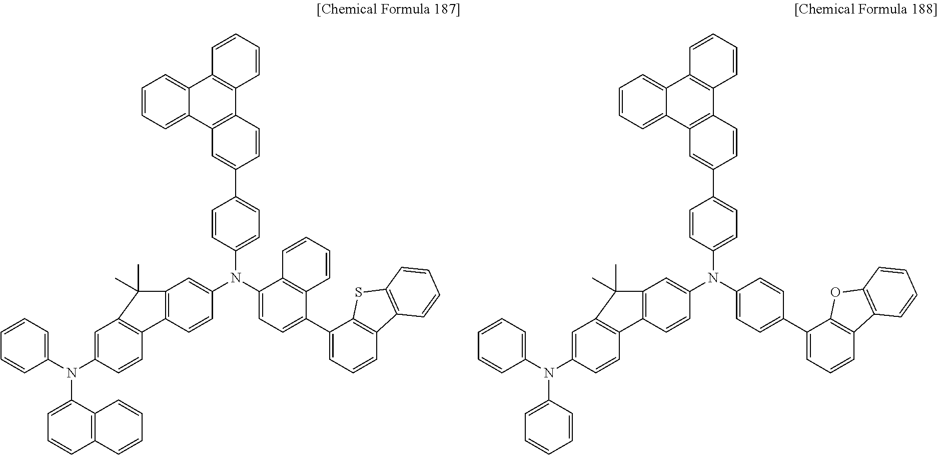 [SMILES: CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4cccc5c4sc4ccccc45)c4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4cccc5c4oc4ccccc45)cc3)cc21]

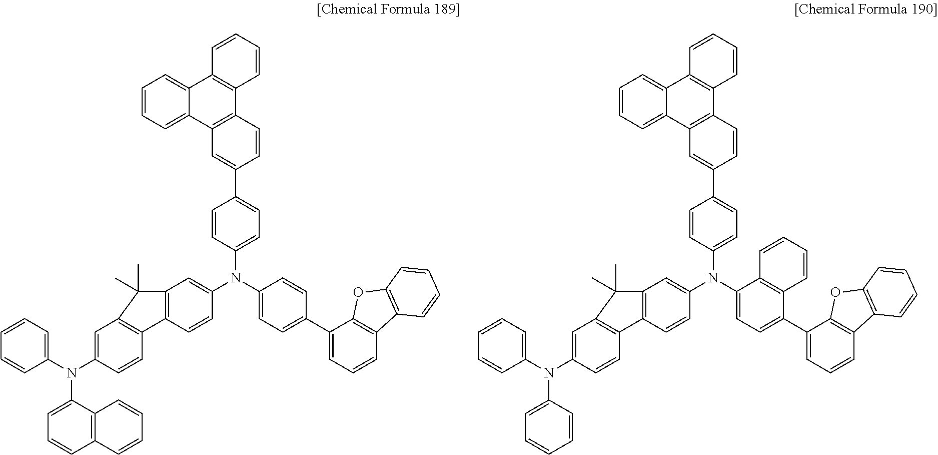 [SMILES: CC1(C)c2cc(N(c3ccc(-c4ccc5c6ccccc6c6ccccc6c5c4)cc3)c3ccc(-c4cccc5c4oc4ccccc45)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4cccc5c4oc4ccccc45)c4ccccc34)cc21]

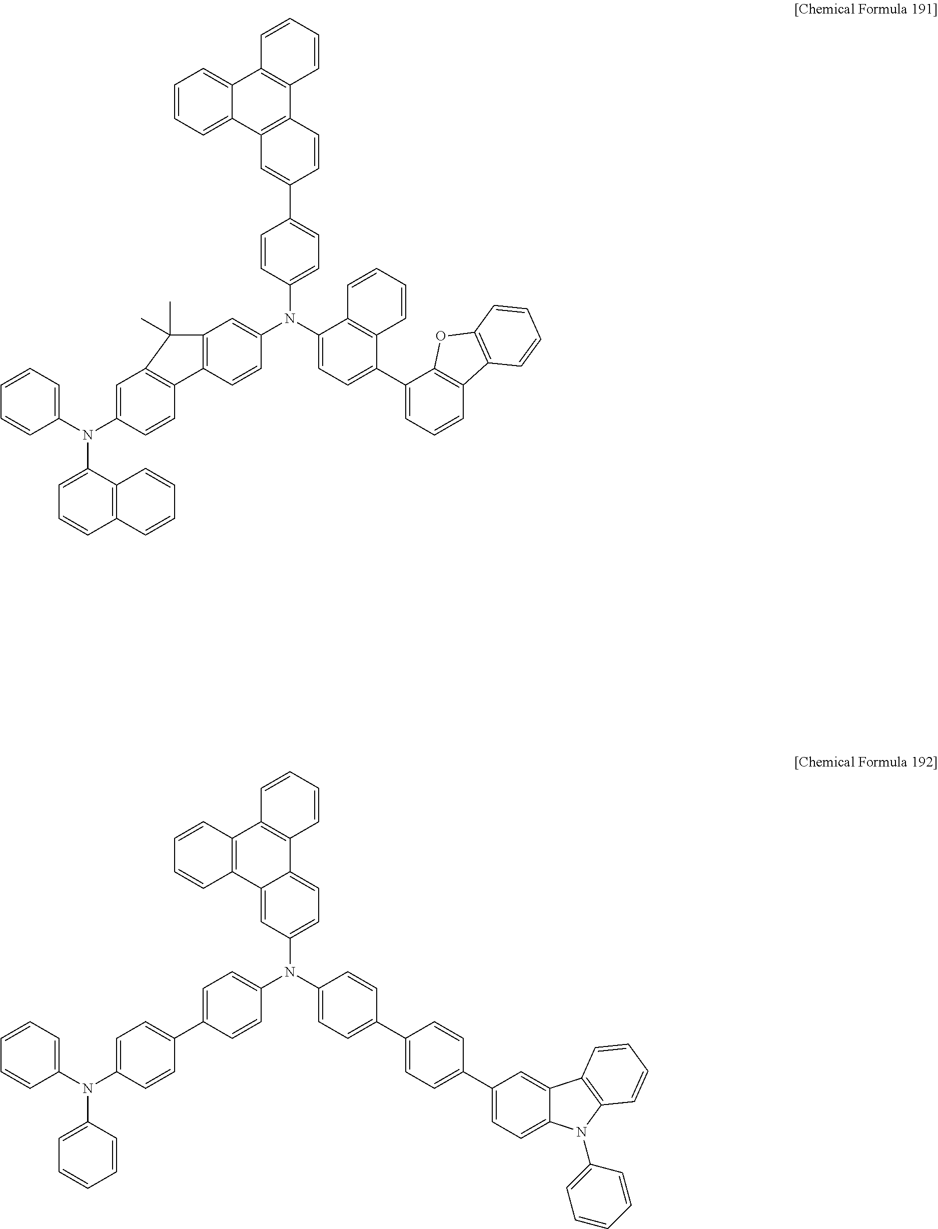 [SMILES: CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4cccc5c4oc4ccccc45)c4ccccc34)cc21 ; c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)cc1]

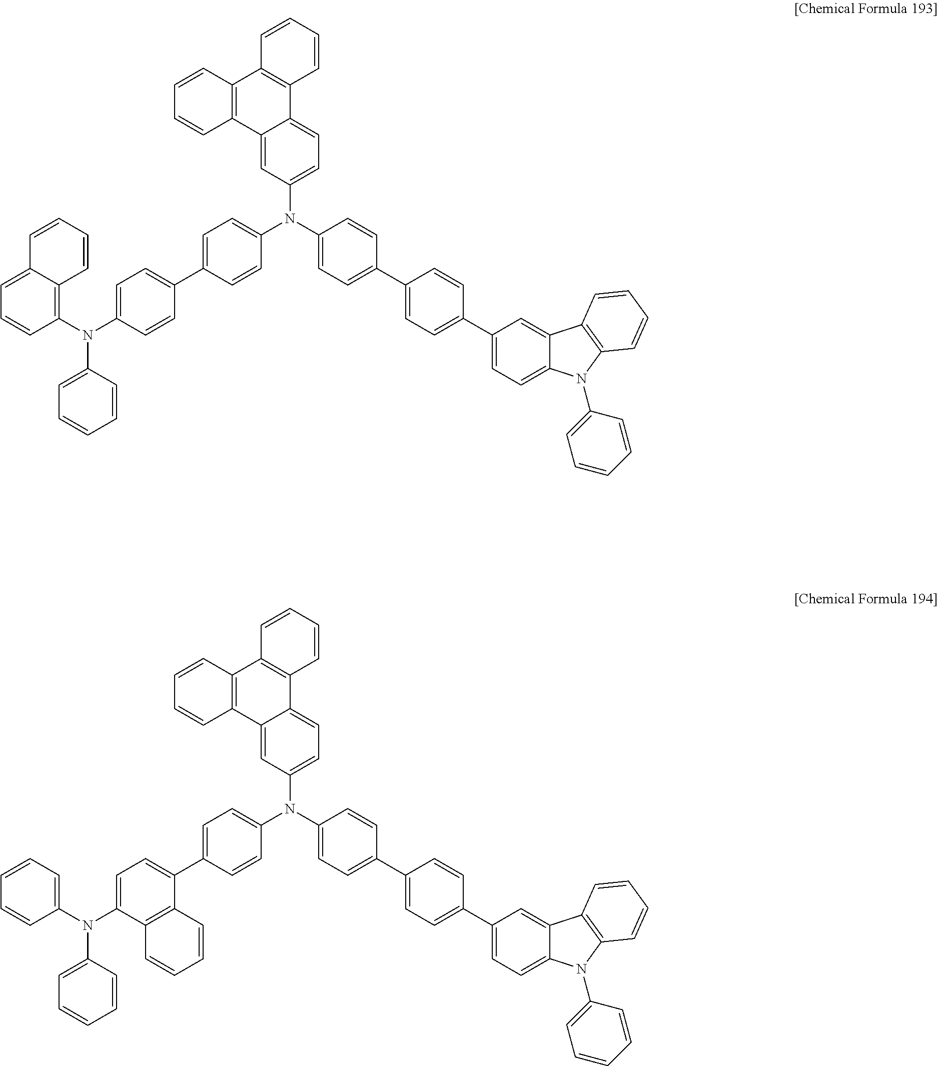 [SMILES: c1ccc(N(c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)c3ccccc23)cc1]

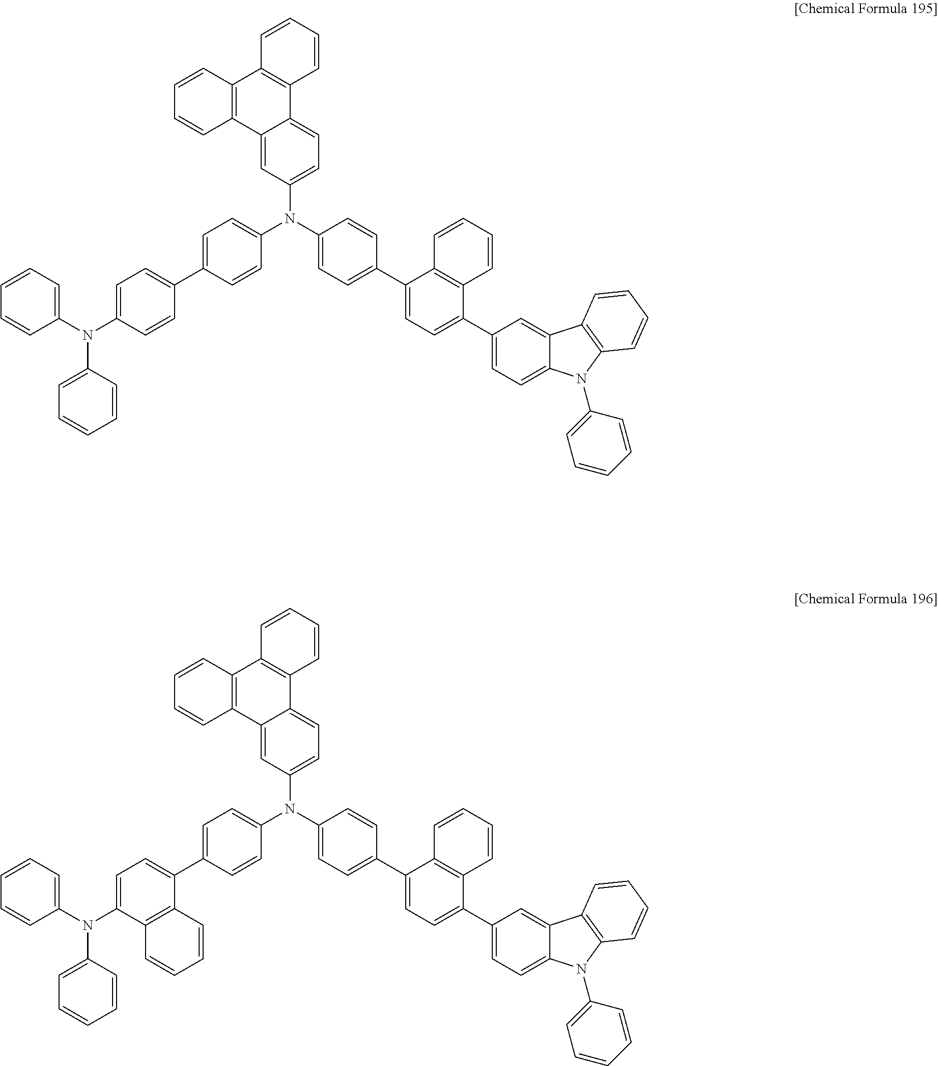 [SMILES: c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)cc2)cc1 ; c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)c3ccccc23)cc1]

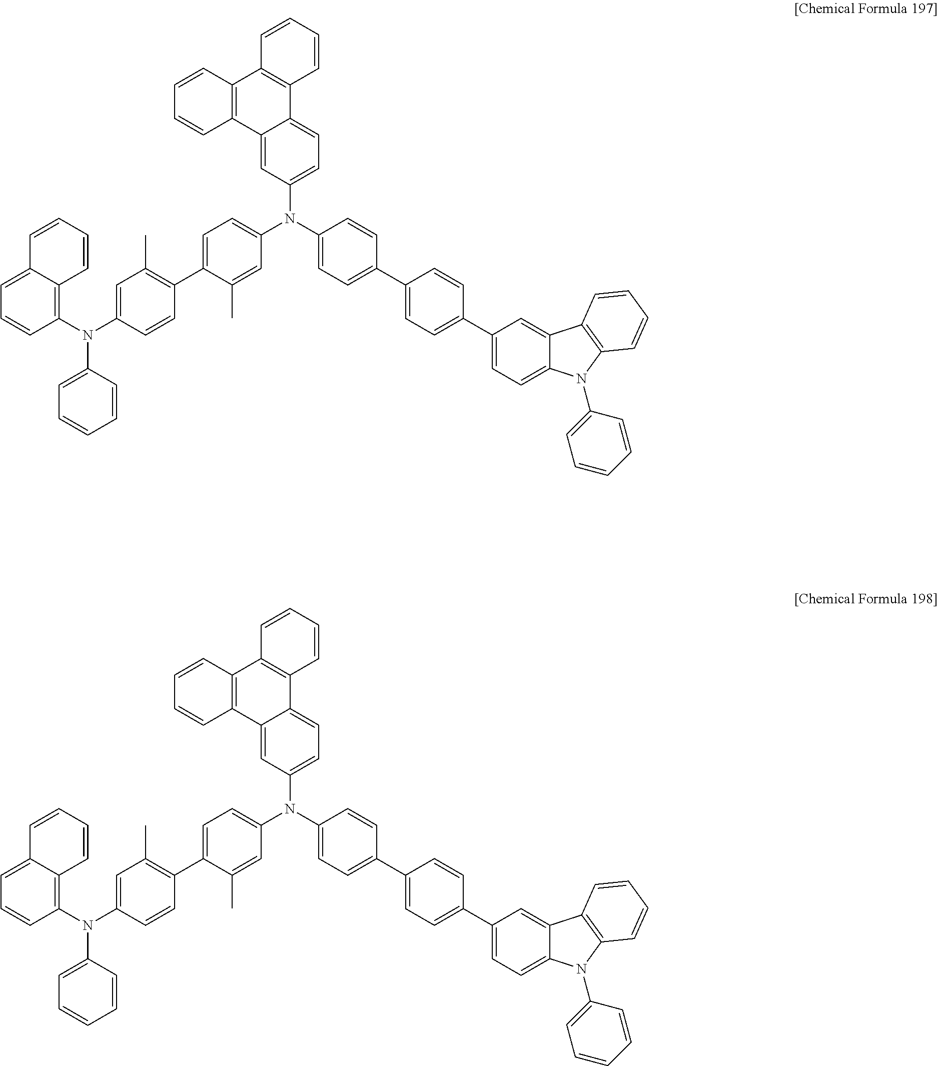 [SMILES: Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C]

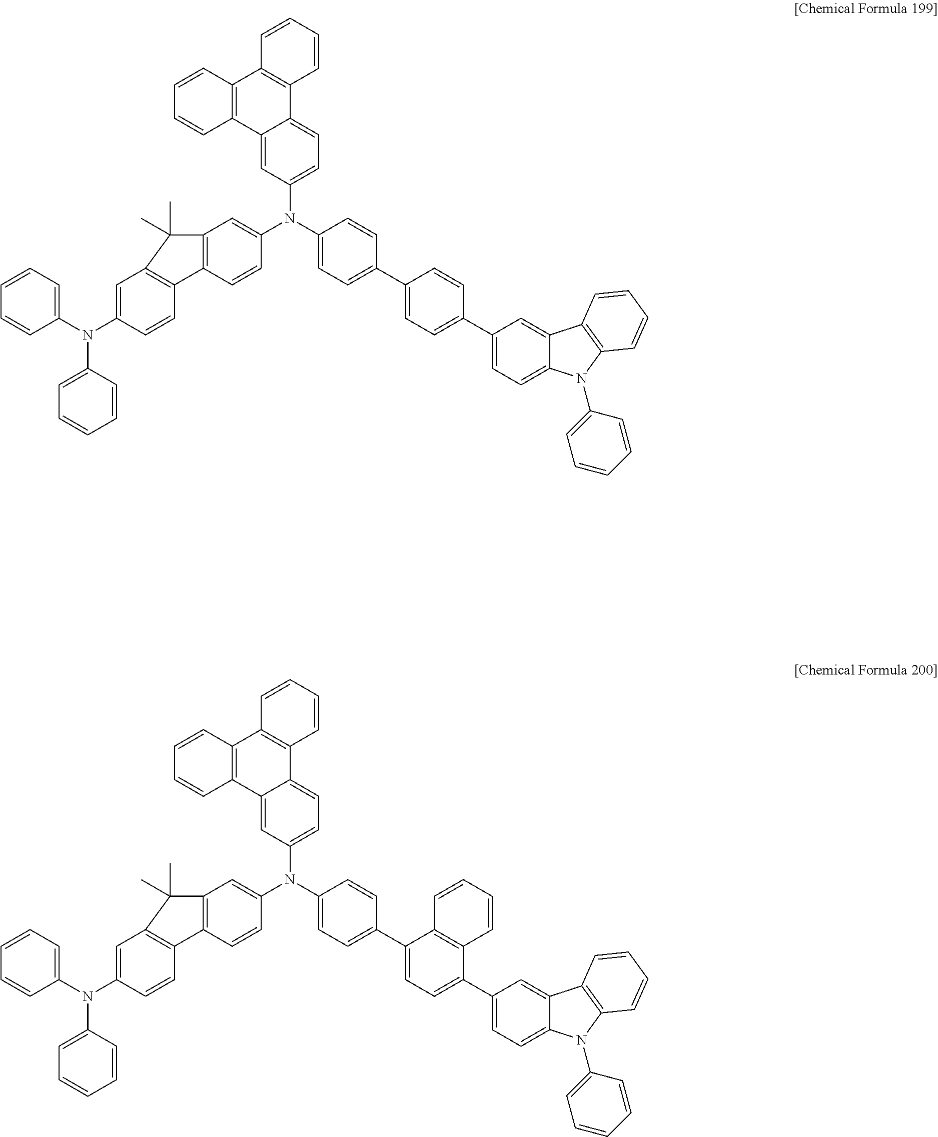 [SMILES: CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc4c5ccccc5c5ccccc5c4c3)cc21 ; CC1(C)c2cc(N(c3ccccc3)c3ccccc3)ccc2-c2ccc(N(c3ccc(-c4ccc(-c5ccc6c(c5)c5ccccc5n6-c5ccccc5)c5ccccc45)cc3)c3ccc4c5ccccc5c5ccccc5c4c3)cc21]

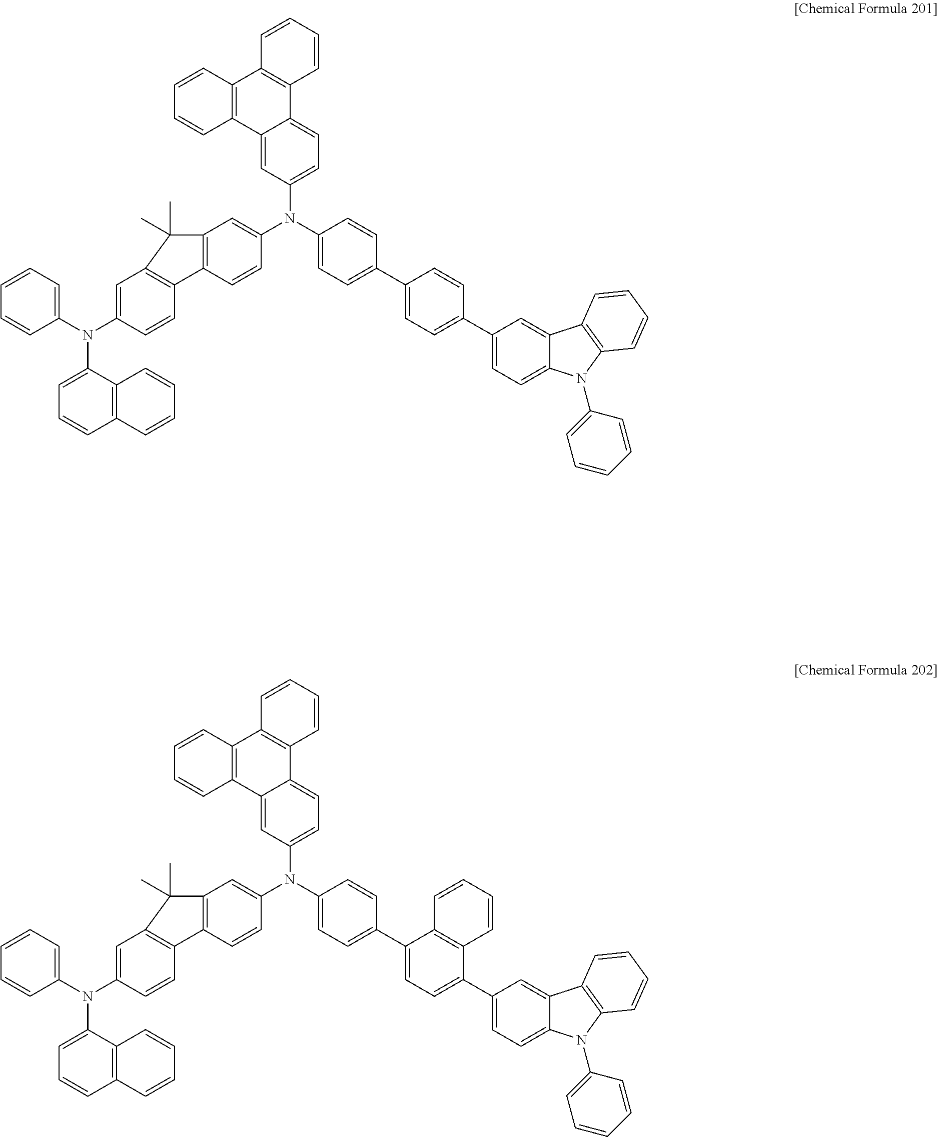 [SMILES: CC1(C)c2cc(N(c3ccc(-c4ccc(-c5ccc6c(c5)c5ccccc5n6-c5ccccc5)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccc(-c4ccc(-c5ccc6c(c5)c5ccccc5n6-c5ccccc5)c5ccccc45)cc3)c3ccc4c5ccccc5c5ccccc5c4c3)ccc2-c2ccc(N(c3ccccc3)c3cccc4ccccc34)cc21]

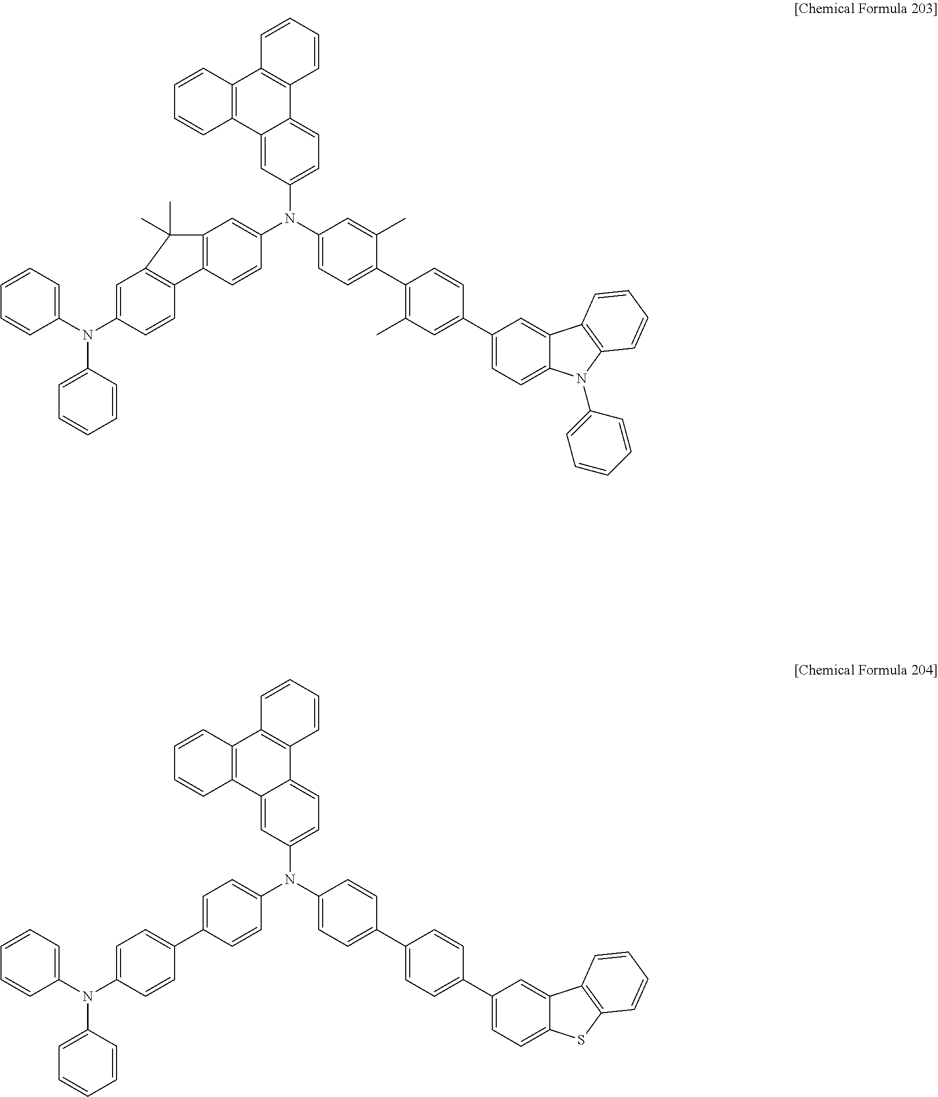 [SMILES: Cc1cc(-c2ccc3c(c2)c2ccccc2n3-c2ccccc2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)cc2)cc1]

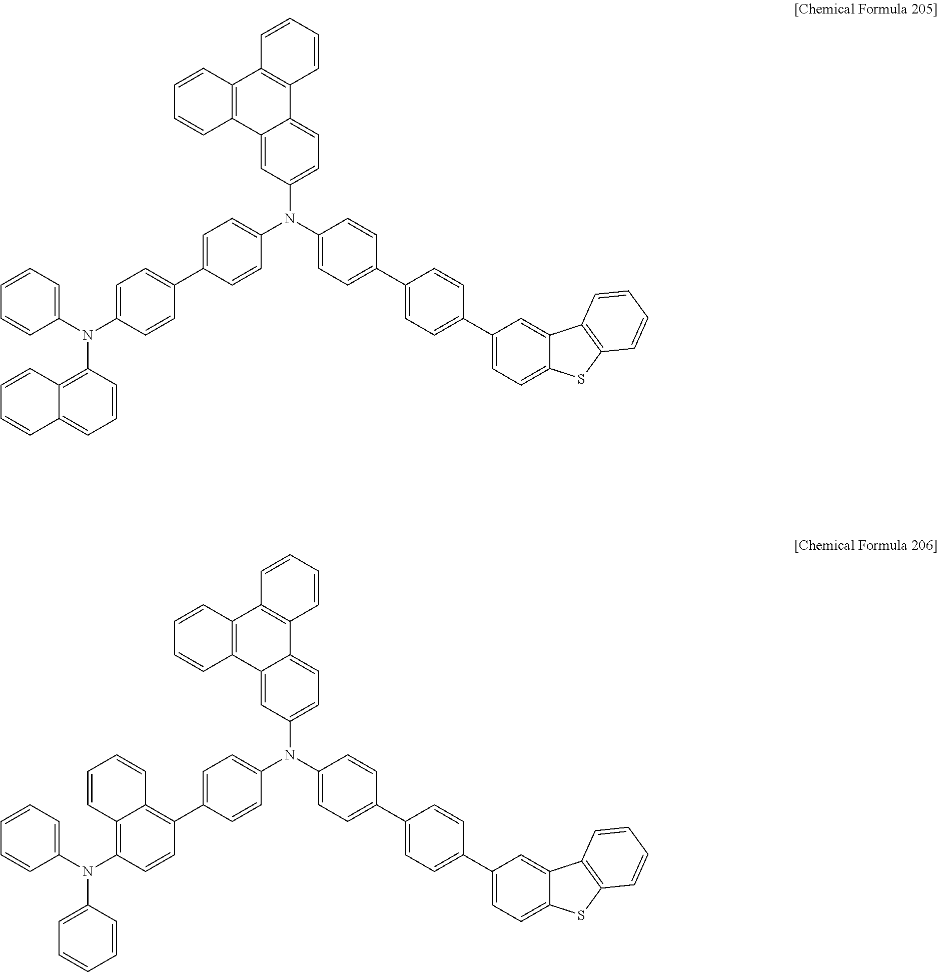 [SMILES: c1ccc(N(c2ccc(-c3ccc(N(c4ccc(-c5ccc(-c6ccc7sc8ccccc8c7c6)cc5)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)c3ccccc23)cc1]

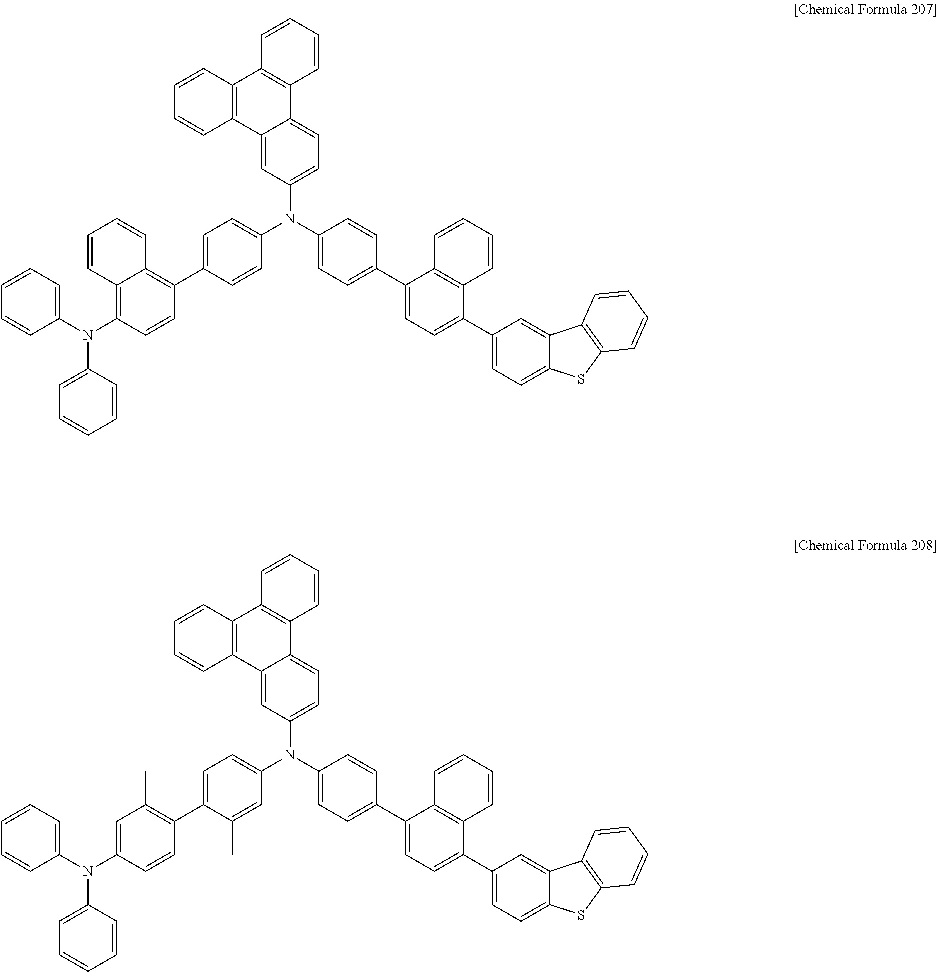 [SMILES: c1ccc(N(c2ccccc2)c2ccc(-c3ccc(N(c4ccc(-c5ccc(-c6ccc7sc8ccccc8c7c6)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4ccc5sc6ccccc6c5c4)c4ccccc34)cc2)c2ccc3c4ccccc4c4ccccc4c3c2)cc1C]

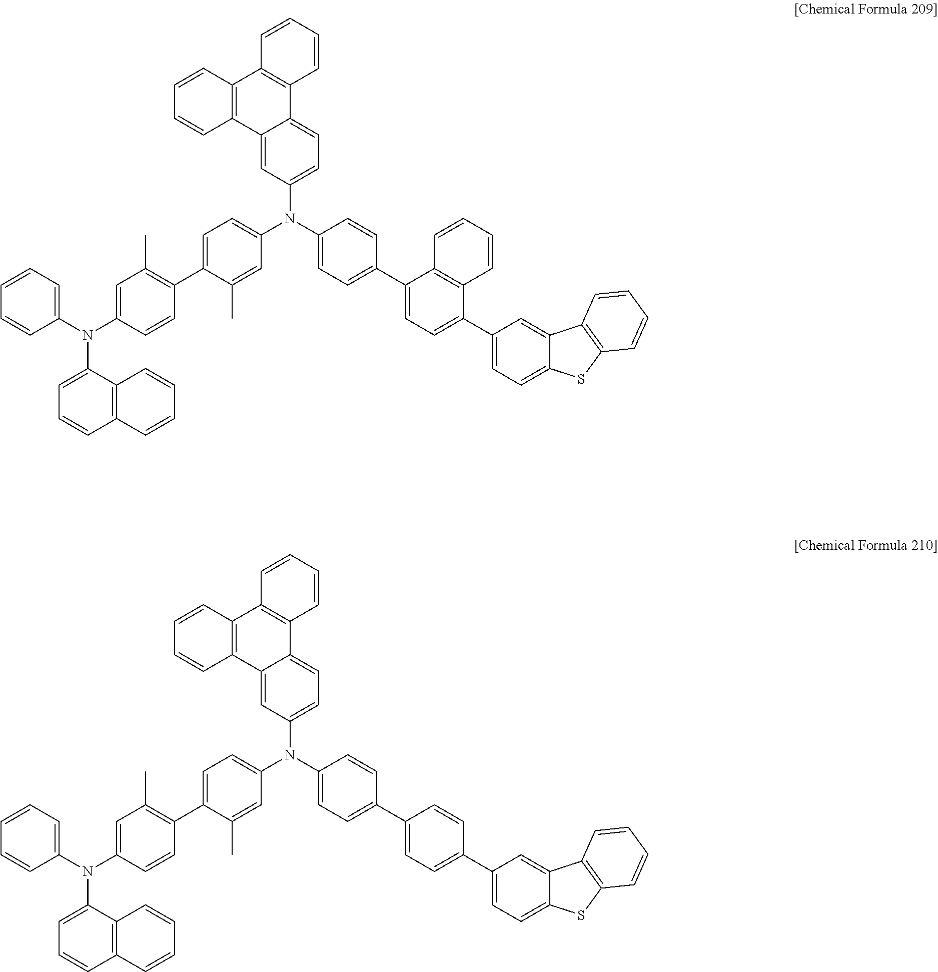 [SMILES: Cc1cc(N(c2ccc(-c3ccc(-c4ccc5sc6ccccc6c5c4)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5sc6ccccc6c5c4)cc3)cc2)c2ccc3c4ccccc4c4ccccc4c3c2)ccc1-c1ccc(N(c2ccccc2)c2cccc3ccccc23)cc1C]

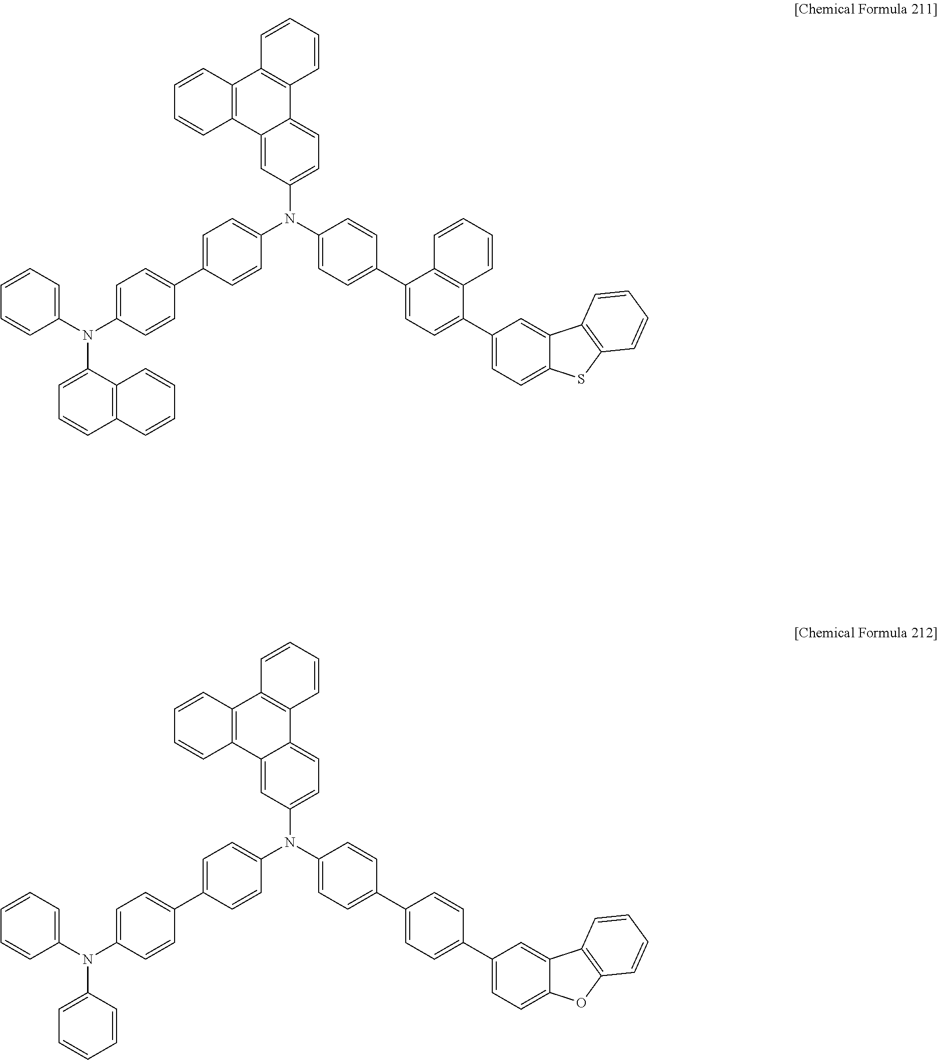 [SMILES: c1ccc(N(c2ccc(-c3ccc(N(c4ccc(-c5ccc(-c6ccc7sc8ccccc8c7c6)c6ccccc56)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)cc2)cc1]

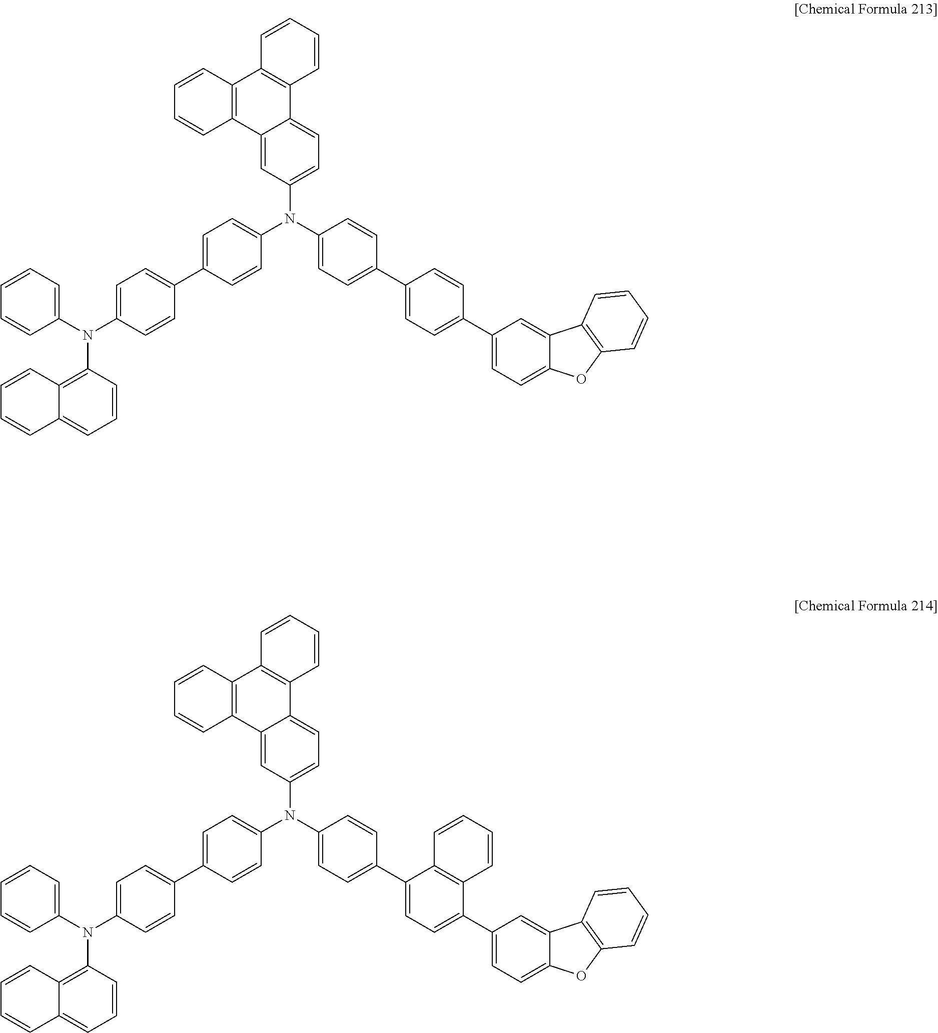 [SMILES: c1ccc(N(c2ccc(-c3ccc(N(c4ccc(-c5ccc(-c6ccc7oc8ccccc8c7c6)cc5)cc4)c4ccc5c6ccccc6c6ccccc6c5c4)cc3)cc2)c2cccc3ccccc23)cc1 ; c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1]

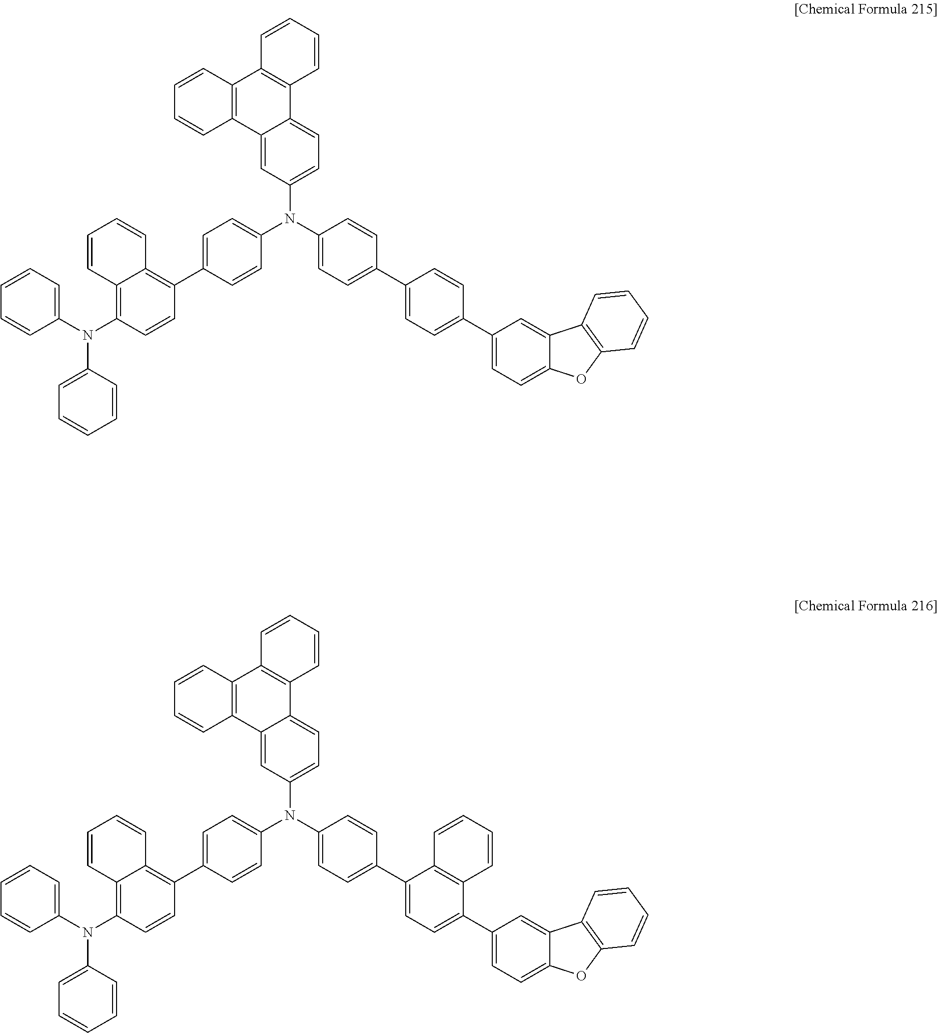 [SMILES: c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7oc8ccccc8c7c6)c6ccccc56)cc4)c4ccc5c6ccccc6c6ccccc6c5c4)cc3)c3ccccc23)cc1]

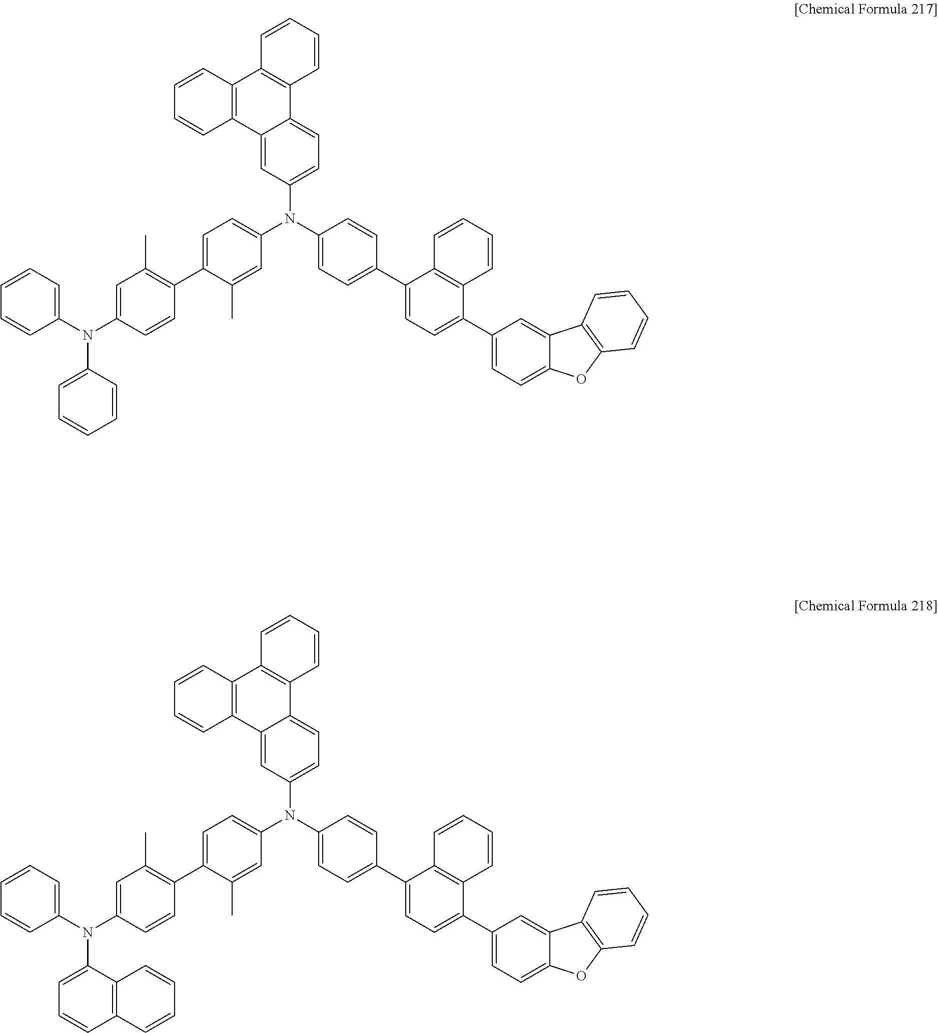 [SMILES: Cc1cc(N(c2ccccc2)c2ccccc2)ccc1-c1ccc(N(c2ccc(-c3ccc(-c4ccc5oc6ccccc6c5c4)c4ccccc34)cc2)c2ccc3c4ccccc4c4ccccc4c3c2)cc1C ; Cc1cc(N(c2ccc(-c3ccc(-c4ccc5oc6ccccc6c5c4)c4ccccc34)cc2)c2ccc3c4ccccc4c4ccccc4c3c2)ccc1-c1ccc(N(c2ccccc2)c2cccc3ccccc23)cc1C]

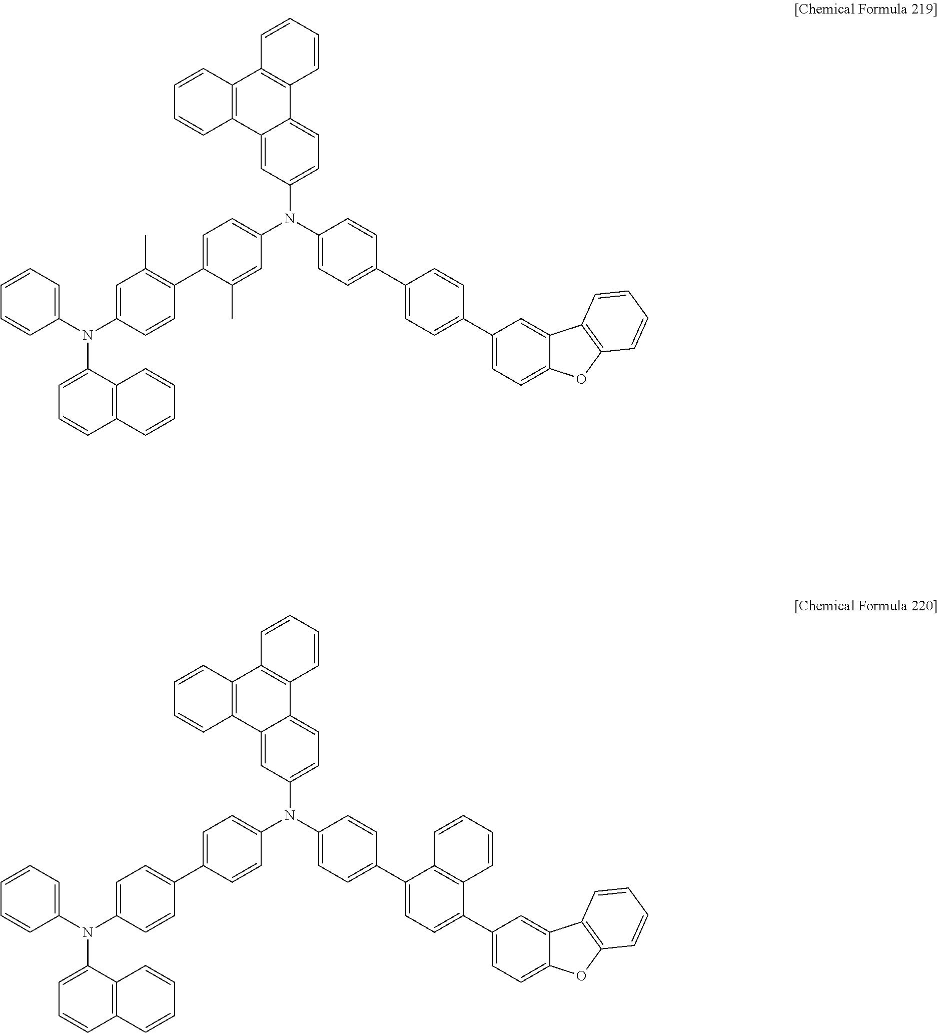 [SMILES: Cc1cc(N(c2ccc(-c3ccc(-c4ccc5oc6ccccc6c5c4)cc3)cc2)c2ccc3c4ccccc4c4ccccc4c3c2)ccc1-c1ccc(N(c2ccccc2)c2cccc3ccccc23)cc1C ; c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1]

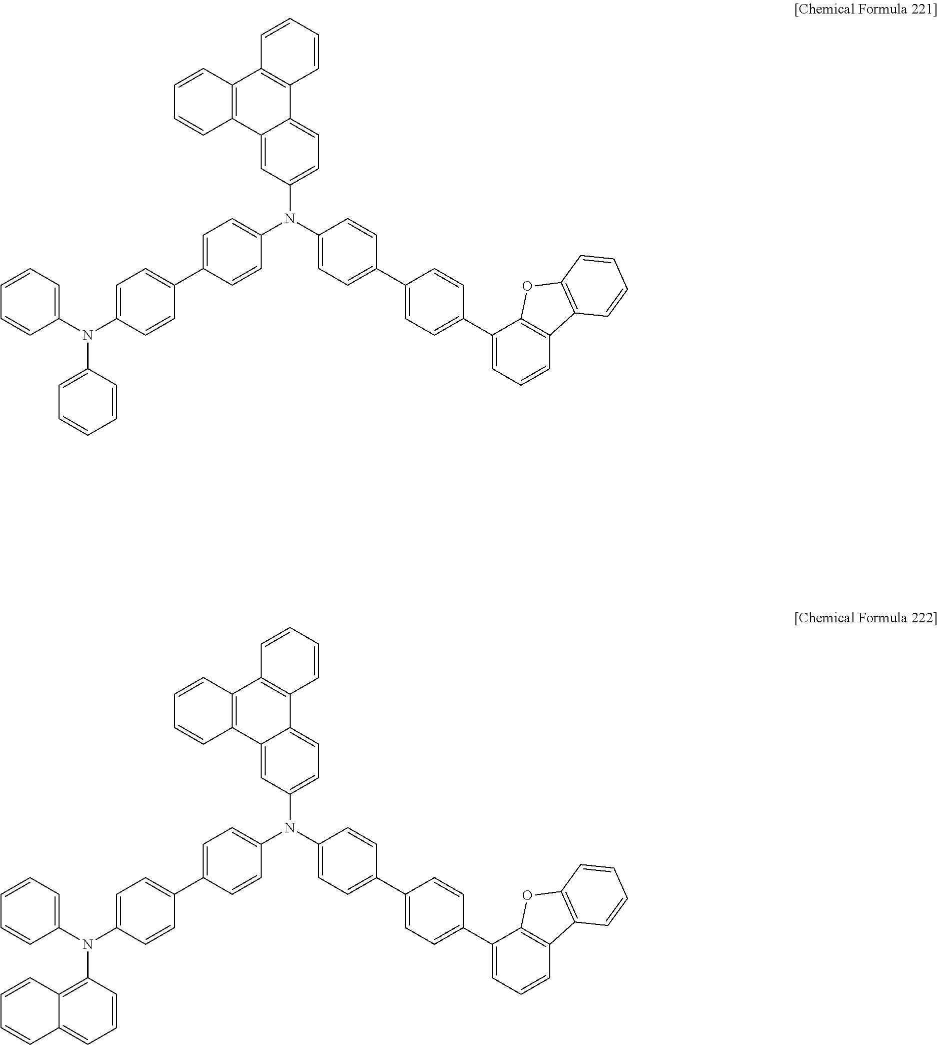 [SMILES: c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)cc2)cc1 ; c1ccc(N(c2ccc(-c3ccc(N(c4ccc(-c5ccc(-c6cccc7c6oc6ccccc67)cc5)cc4)c4ccc5c6ccccc6c6ccccc6c5c4)cc3)cc2)c2cccc3ccccc23)cc1]

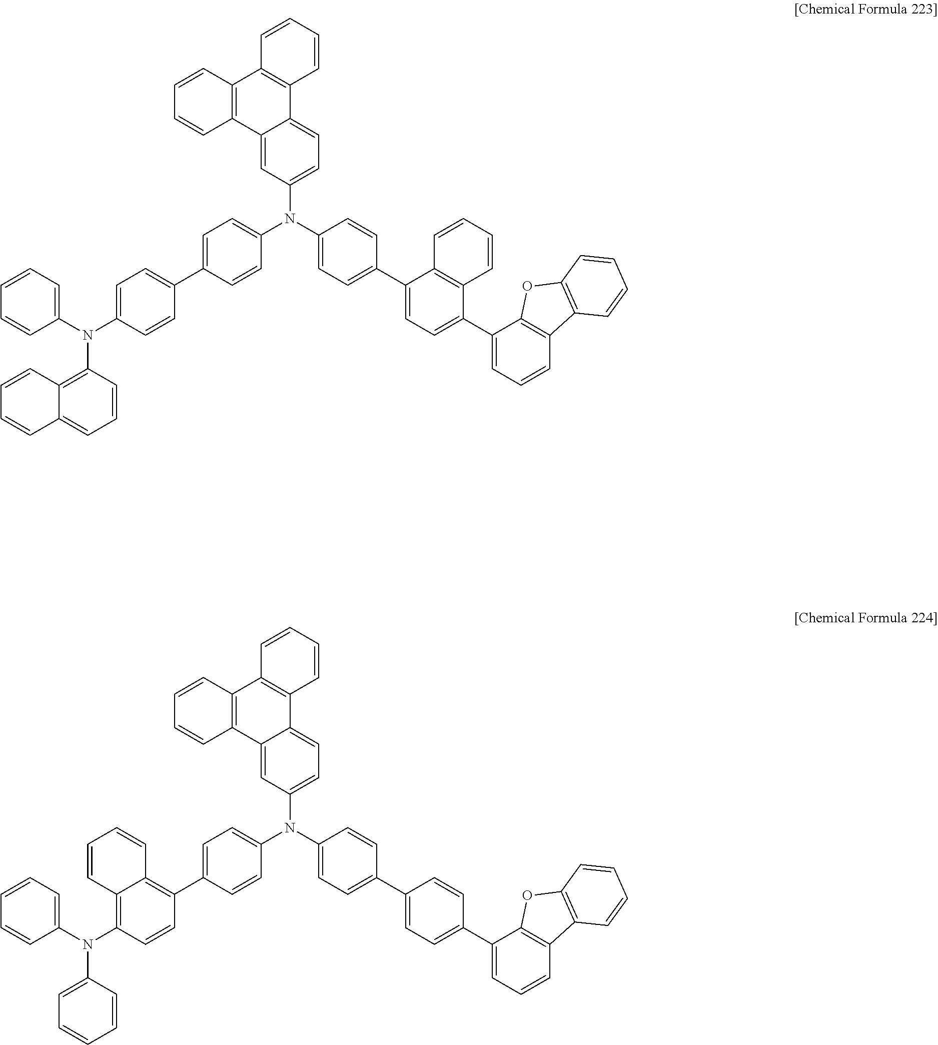 [SMILES: c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1 ; c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)c3ccccc23)cc1]

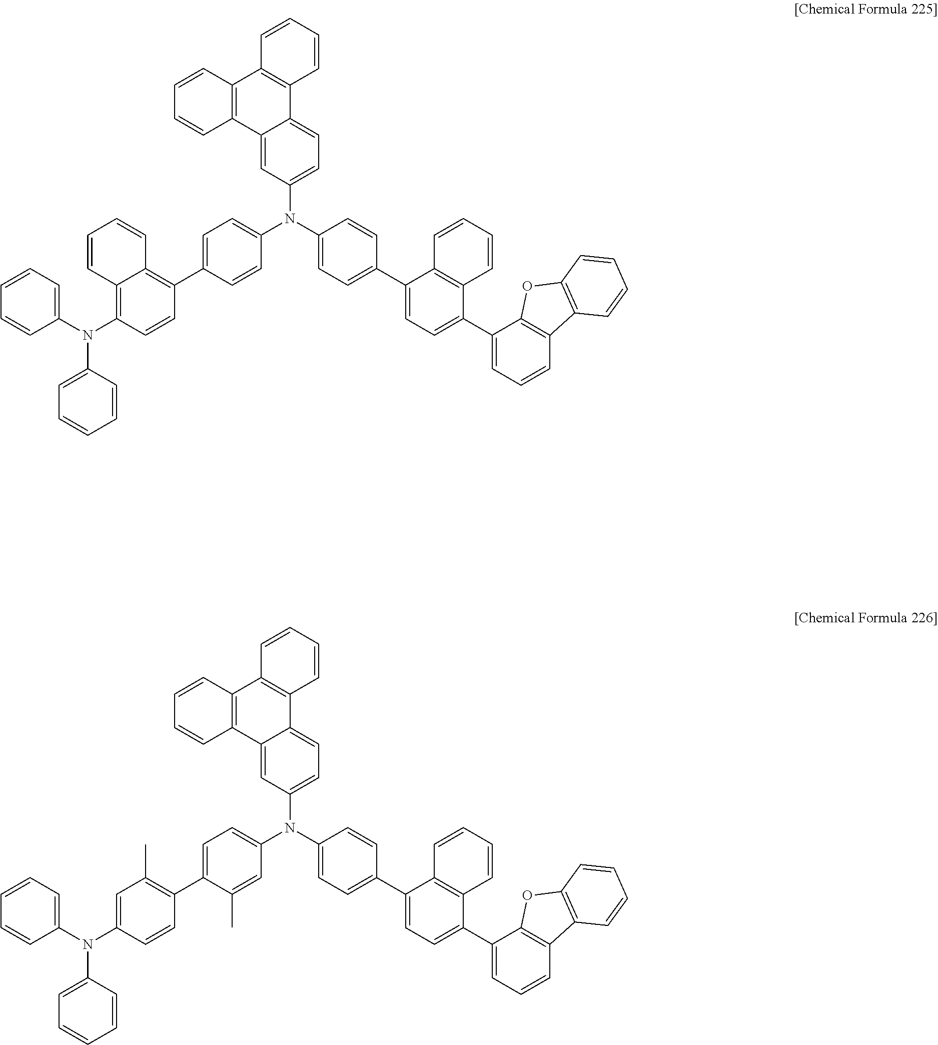 [SMILES: c1ccc(N(c2ccccc2)c2ccc(-c3ccc(N(c4ccc(-c5ccc(-c6cccc7c6oc6ccccc67)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4cccc5c4oc4ccccc45)c4ccccc34)cc2)c2ccc3c4ccccc4c4ccccc4c3c2)cc1C]

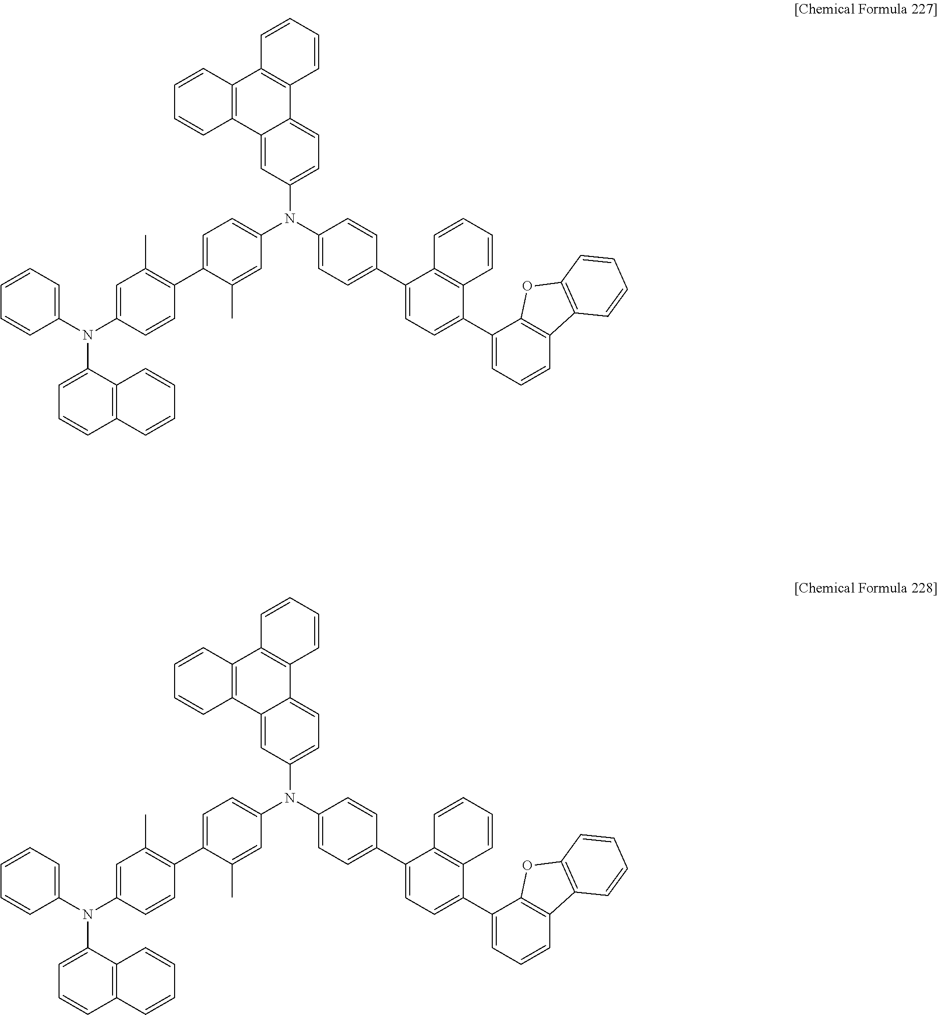 [SMILES: Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C]

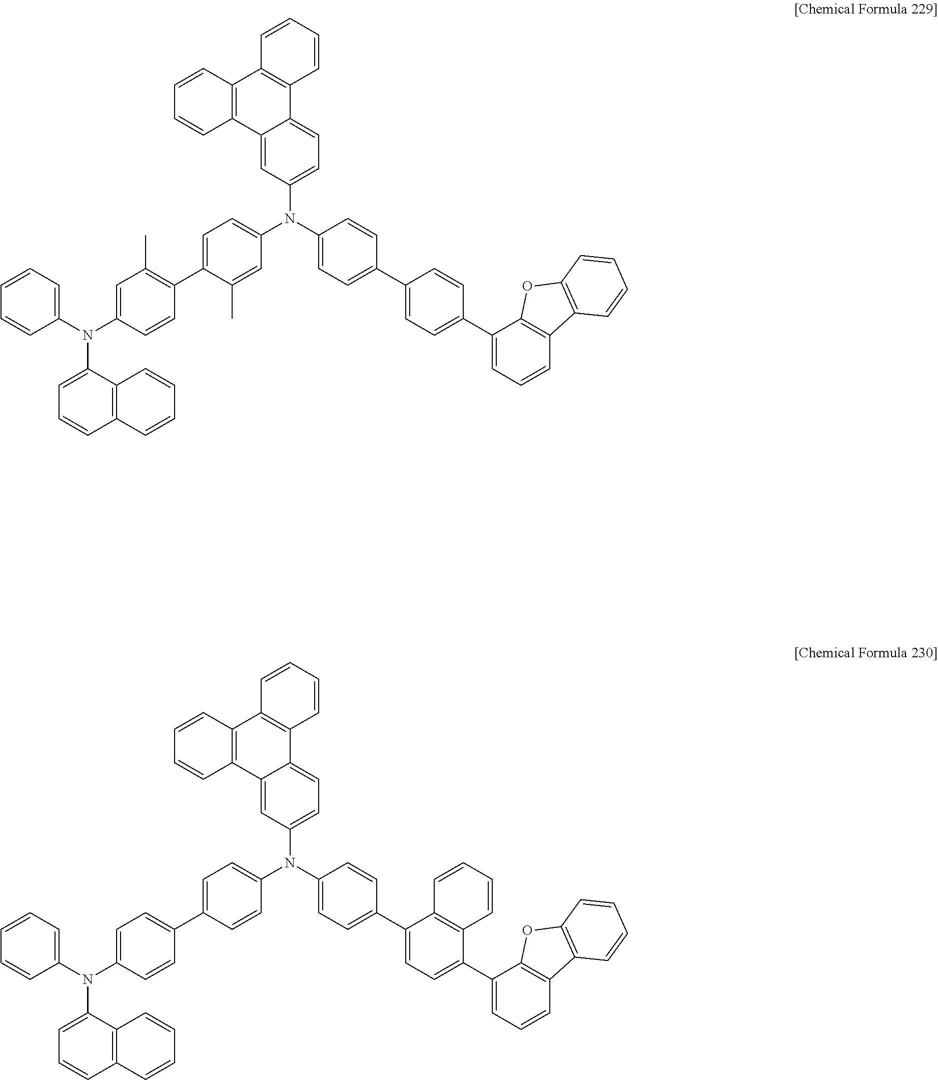 [SMILES: Cc1cc(N(c2ccc(-c3ccc(-c4cccc5c4oc4ccccc45)cc3)cc2)c2ccc3c4ccccc4c4ccccc4c3c2)ccc1-c1ccc(N(c2ccccc2)c2cccc3ccccc23)cc1C ; c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1]

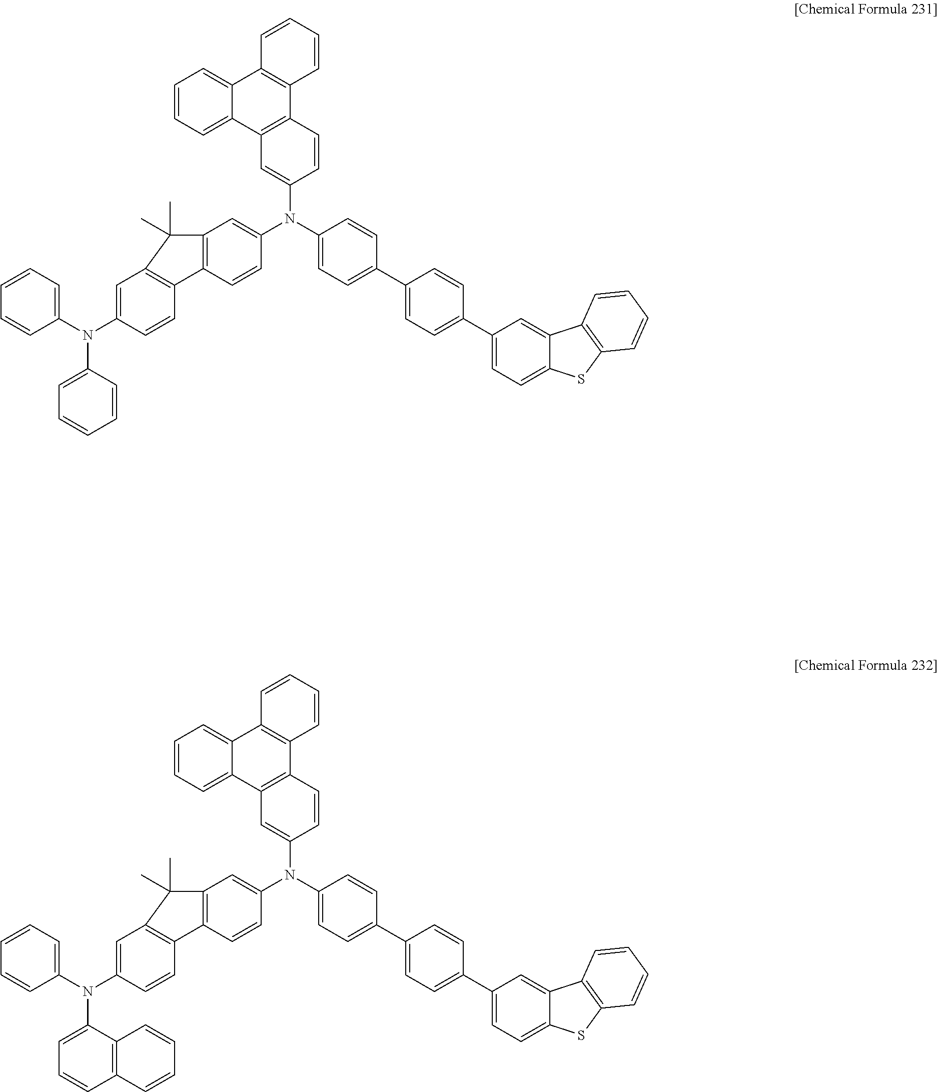 [SMILES: CC1(C)c2cc(N(c3ccccc3)c3ccccc3)ccc2-c2ccc(N(c3ccc(-c4ccc(-c5ccc6sc7ccccc7c6c5)cc4)cc3)c3ccc4c5ccccc5c5ccccc5c4c3)cc21 ; CC1(C)c2cc(N(c3ccc(-c4ccc(-c5ccc6sc7ccccc7c6c5)cc4)cc3)c3ccc4c5ccccc5c5ccccc5c4c3)ccc2-c2ccc(N(c3ccccc3)c3cccc4ccccc34)cc21]

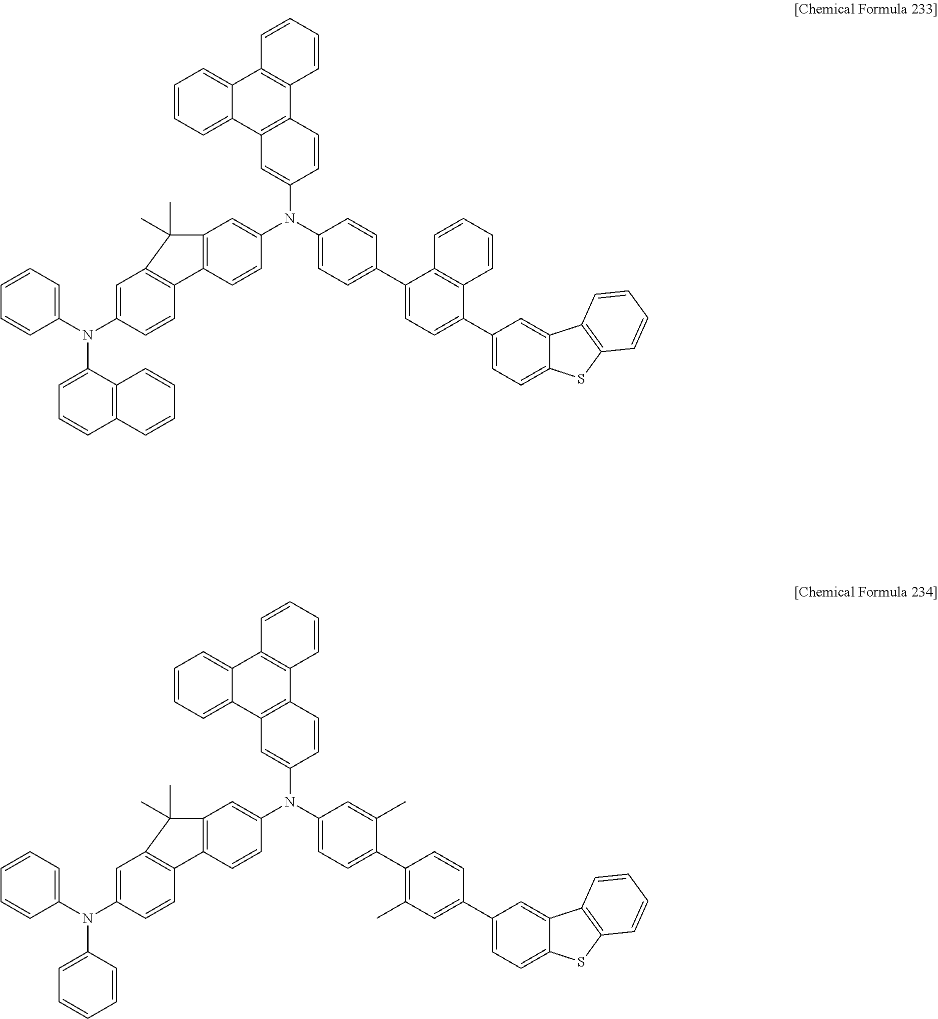 [SMILES: CC1(C)c2cc(N(c3ccc(-c4ccc(-c5ccc6sc7ccccc7c6c5)c5ccccc45)cc3)c3ccc4c5ccccc5c5ccccc5c4c3)ccc2-c2ccc(N(c3ccccc3)c3cccc4ccccc34)cc21 ; Cc1cc(-c2ccc3sc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C]

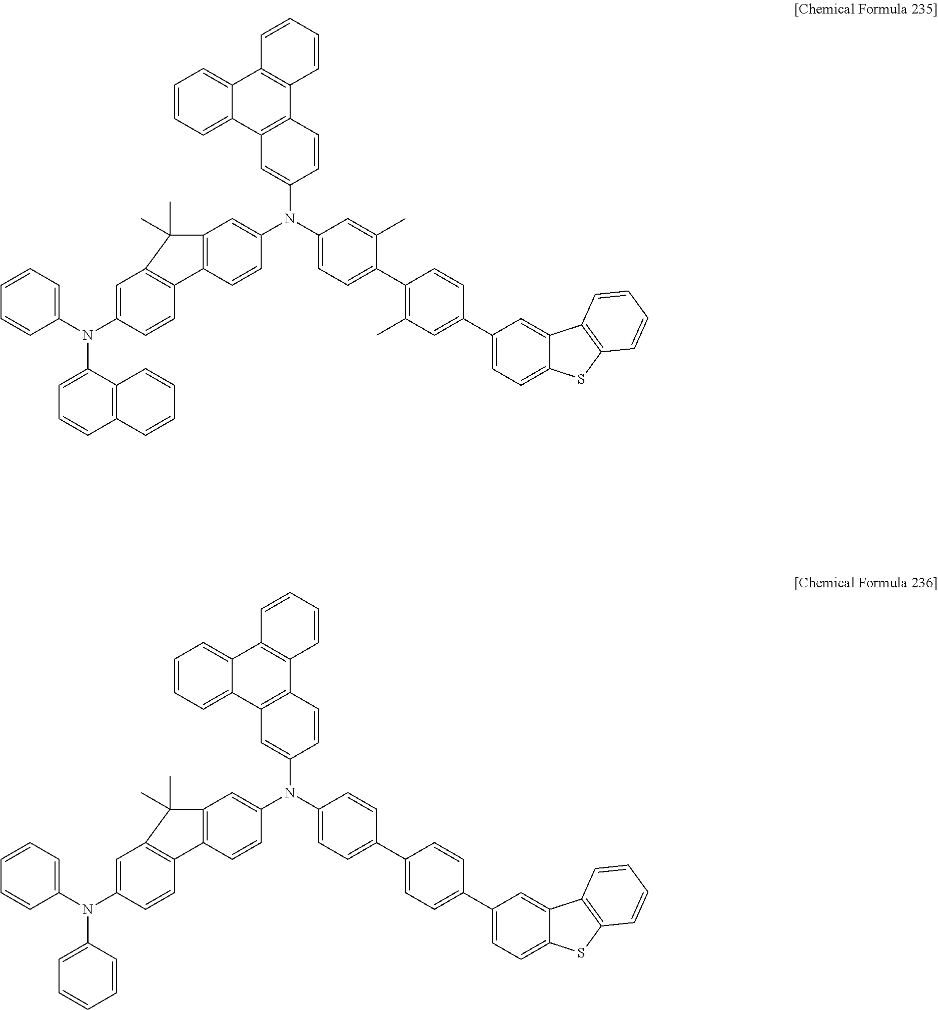 [SMILES: Cc1cc(-c2ccc3sc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C ; CC1(C)c2cc(N(c3ccccc3)c3ccccc3)ccc2-c2ccc(N(c3ccc(-c4ccc(-c5ccc6sc7ccccc7c6c5)cc4)cc3)c3ccc4c5ccccc5c5ccccc5c4c3)cc21]

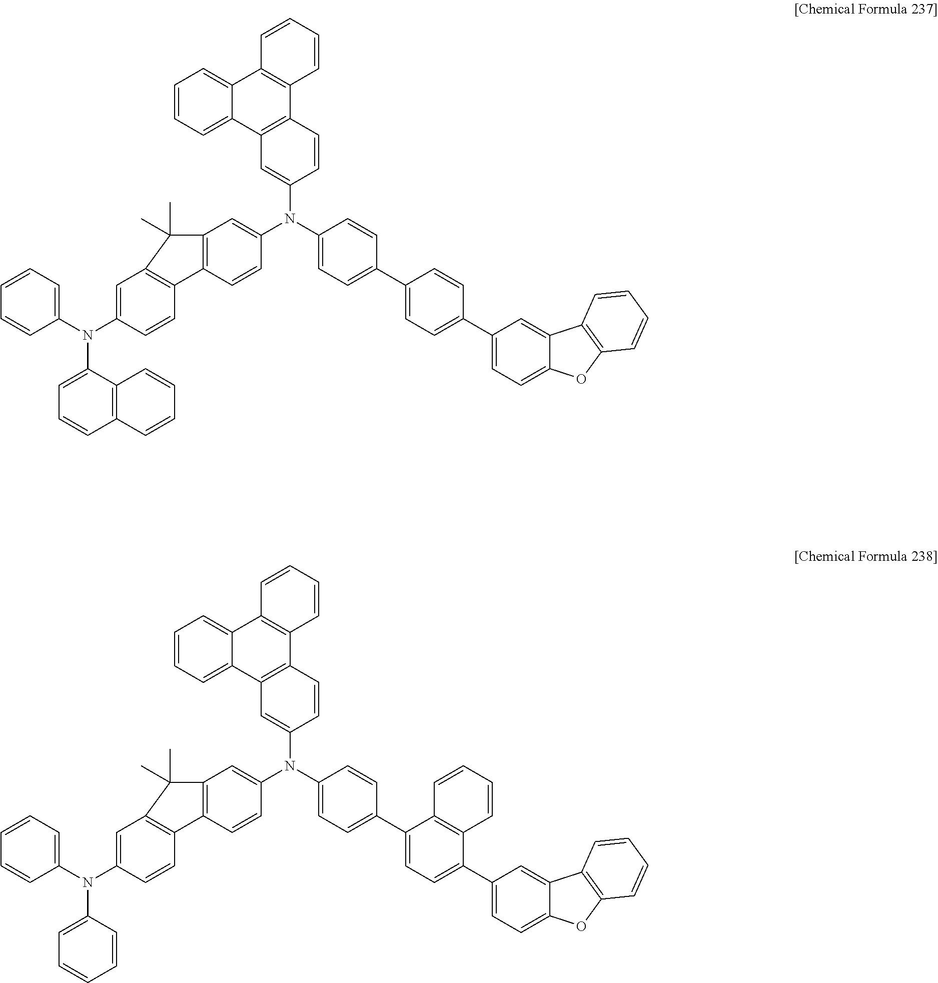 [SMILES: CC1(C)c2cc(N(c3ccc(-c4ccc(-c5ccc6oc7ccccc7c6c5)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5ccc6oc7ccccc7c6c5)c5ccccc45)cc3)c3ccc4c5ccccc5c5ccccc5c4c3)cc21]

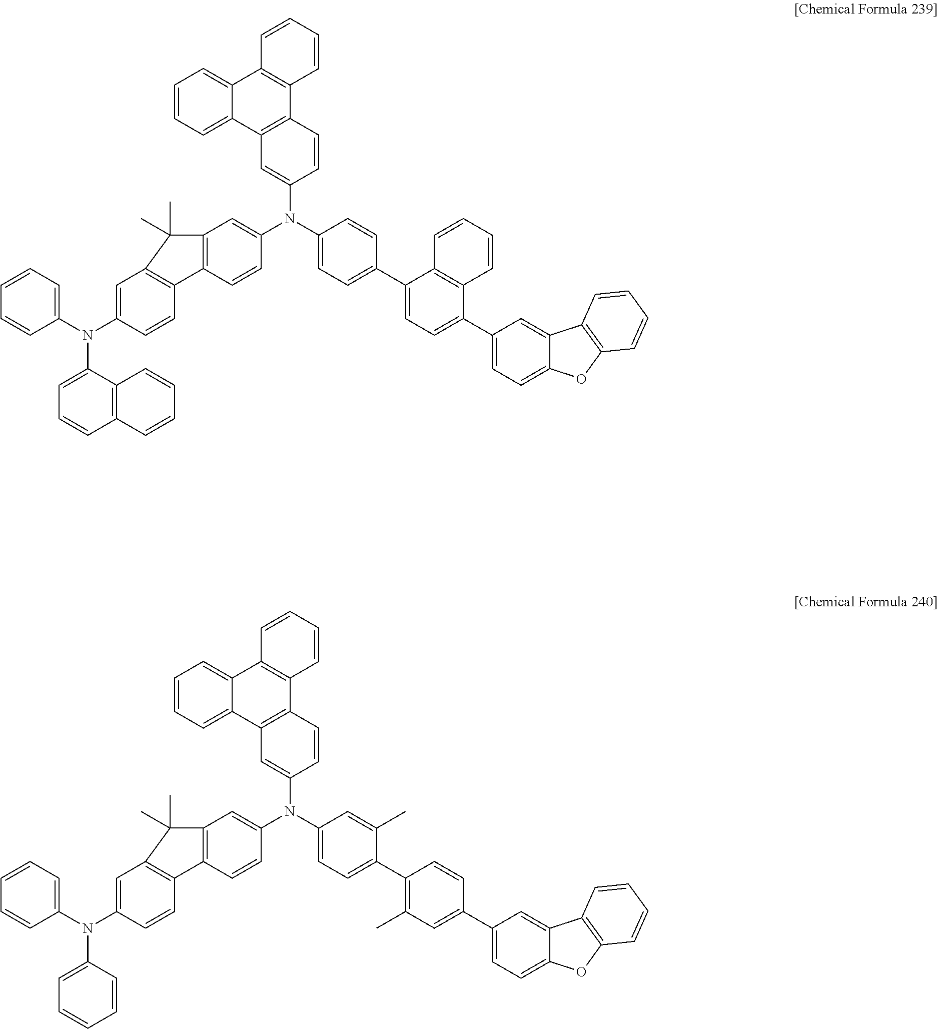 [SMILES: CC1(C)c2cc(N(c3ccc(-c4ccc(-c5ccc6oc7ccccc7c6c5)c5ccccc45)cc3)c3ccc4c5ccccc5c5ccccc5c4c3)ccc2-c2ccc(N(c3ccccc3)c3cccc4ccccc34)cc21 ; Cc1cc(-c2ccc3oc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C]

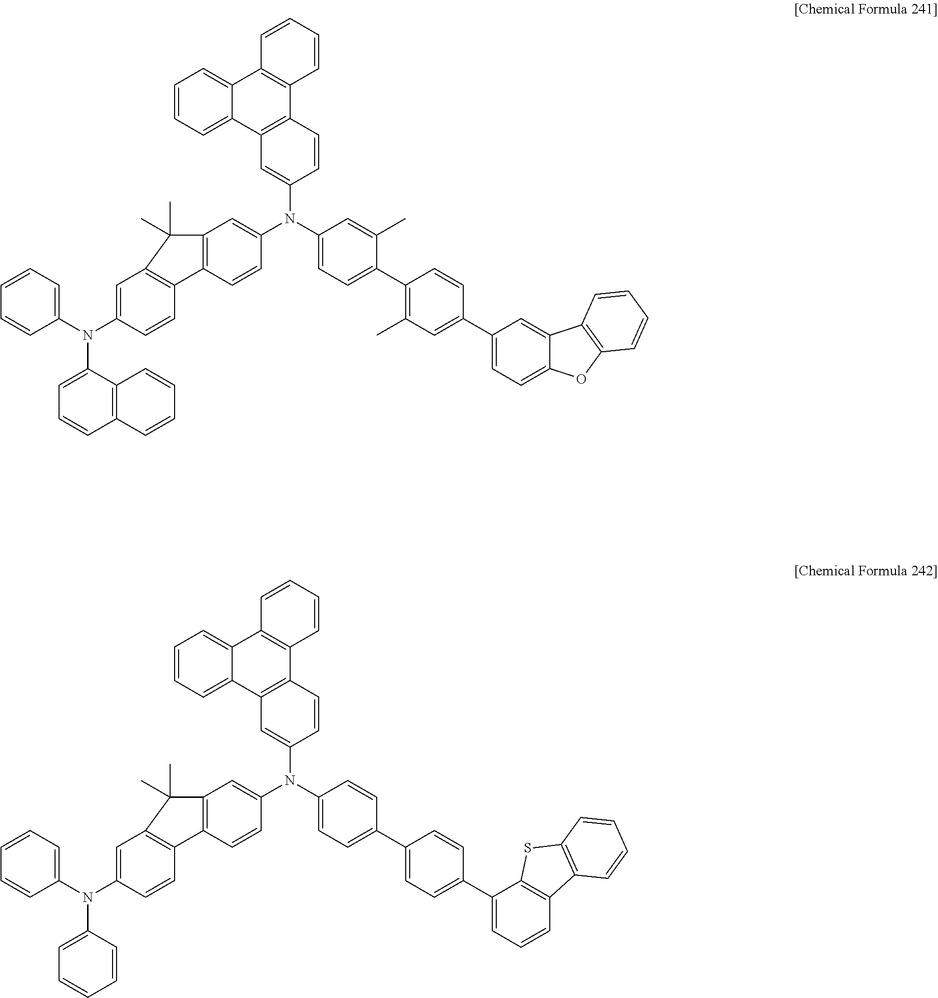 [SMILES: Cc1cc(-c2ccc3oc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C ; CC1(C)c2cc(N(c3ccccc3)c3ccccc3)ccc2-c2ccc(N(c3ccc(-c4ccc(-c5cccc6c5sc5ccccc56)cc4)cc3)c3ccc4c5ccccc5c5ccccc5c4c3)cc21]

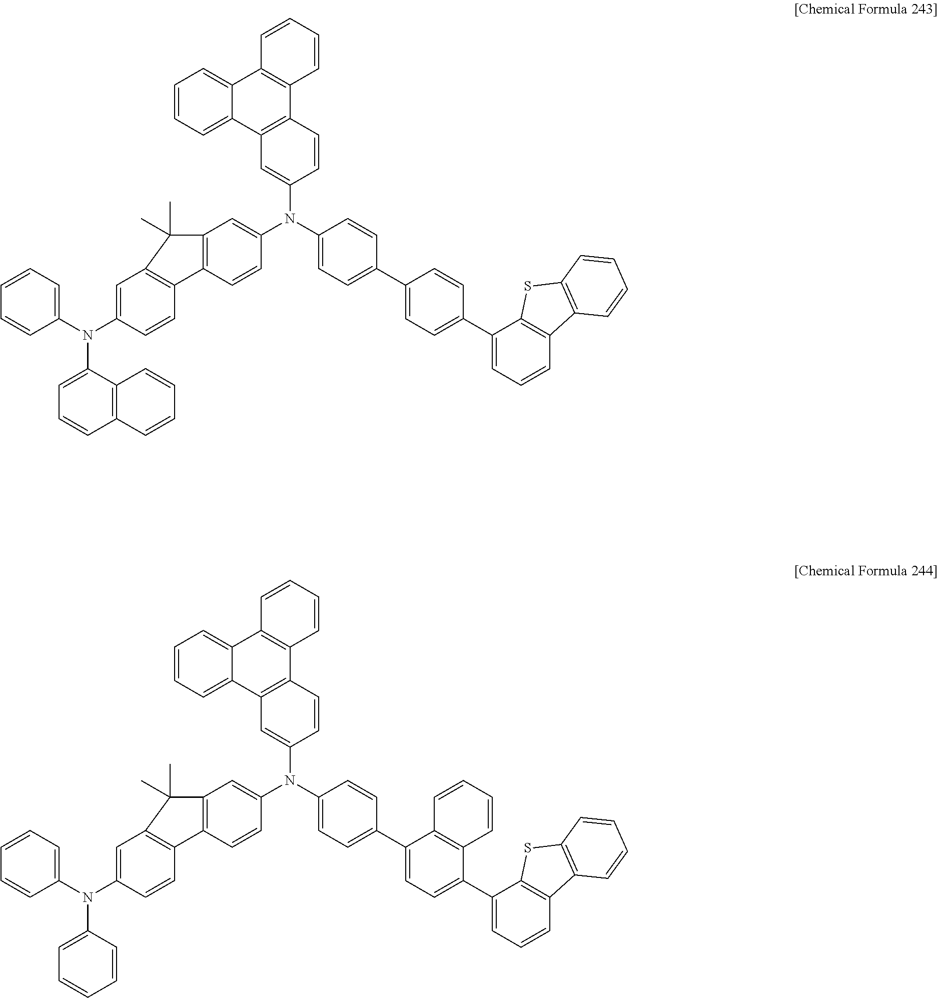 [SMILES: CC1(C)c2cc(N(c3ccc(-c4ccc(-c5cccc6c5sc5ccccc56)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5cccc6c5sc5ccccc56)c5ccccc45)cc3)c3ccc4c5ccccc5c5ccccc5c4c3)cc21]

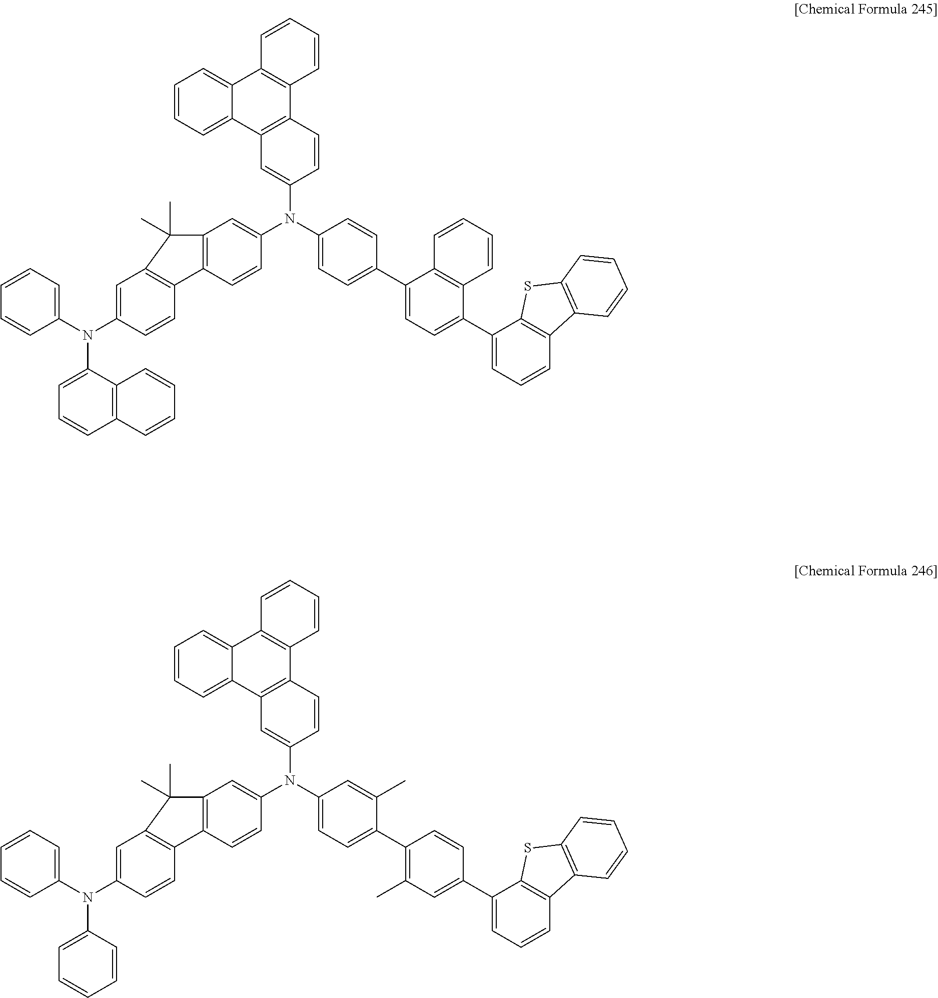 [SMILES: CC1(C)c2cc(N(c3ccc(-c4ccc(-c5cccc6c5sc5ccccc56)c5ccccc45)cc3)c3ccc4c5ccccc5c5ccccc5c4c3)ccc2-c2ccc(N(c3ccccc3)c3cccc4ccccc34)cc21 ; Cc1cc(-c2cccc3c2sc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C]

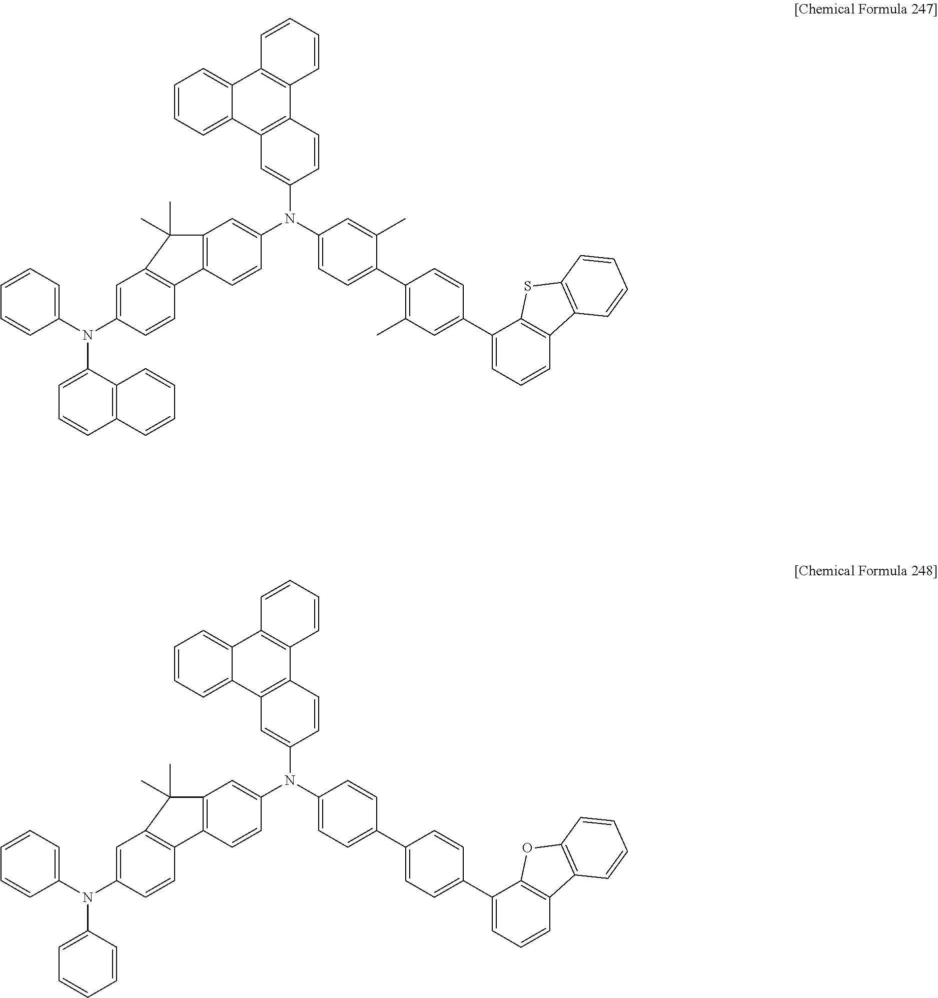 [SMILES: Cc1cc(-c2cccc3c2sc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C ; CC1(C)c2cc(N(c3ccccc3)c3ccccc3)ccc2-c2ccc(N(c3ccc(-c4ccc(-c5cccc6c5oc5ccccc56)cc4)cc3)c3ccc4c5ccccc5c5ccccc5c4c3)cc21]

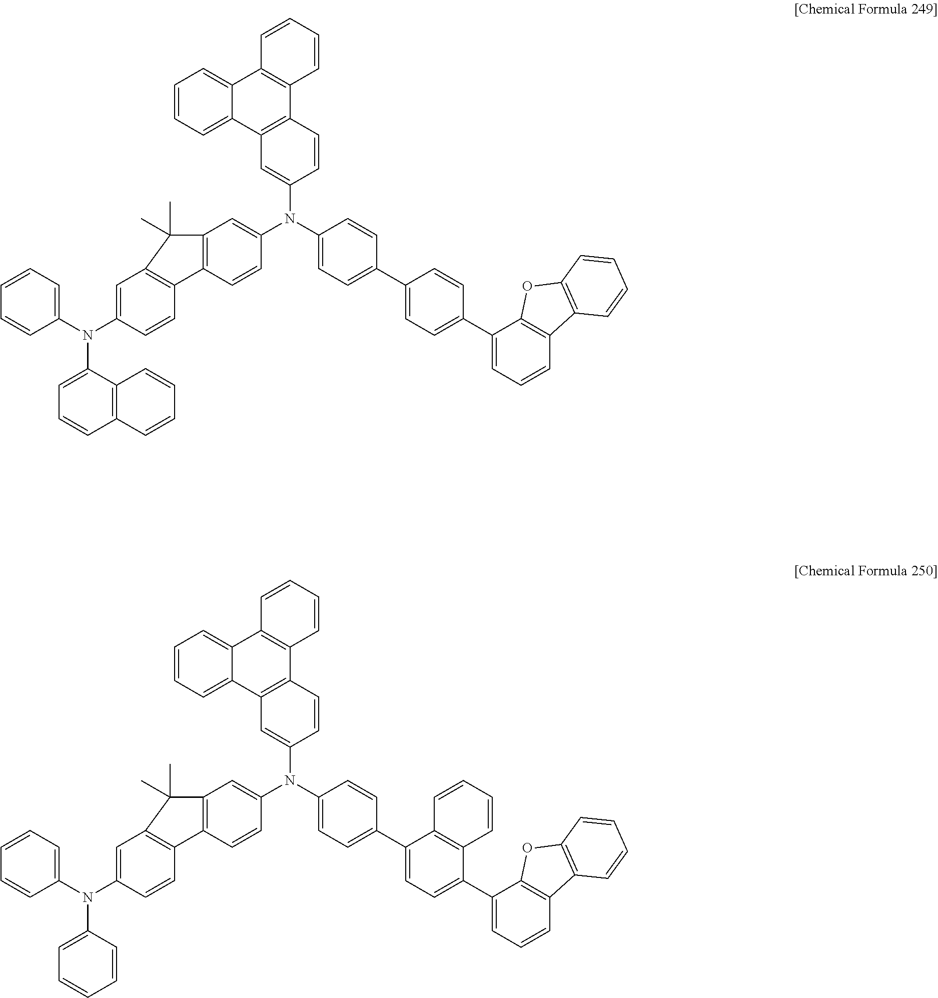 [SMILES: CC1(C)c2cc(N(c3ccc(-c4ccc(-c5cccc6c5oc5ccccc56)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5cccc6c5oc5ccccc56)c5ccccc45)cc3)c3ccc4c5ccccc5c5ccccc5c4c3)cc21]

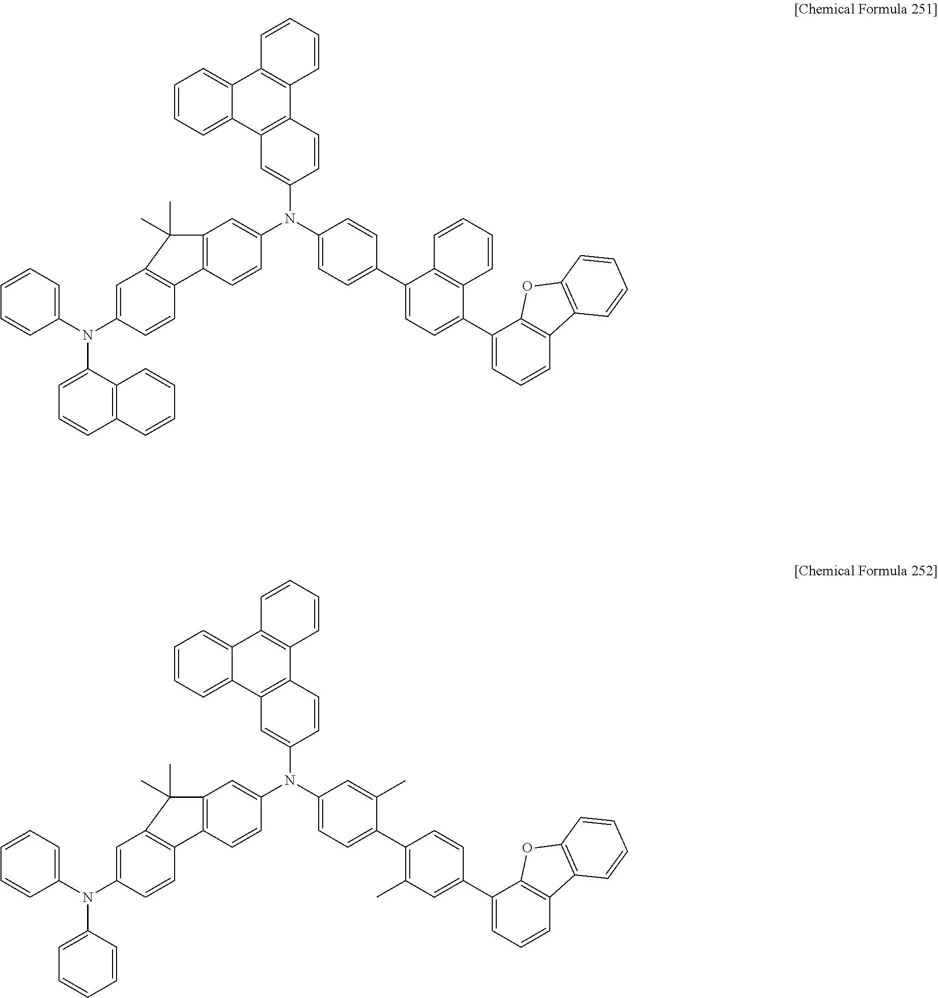 [SMILES: CC1(C)c2cc(N(c3ccc(-c4ccc(-c5cccc6c5oc5ccccc56)c5ccccc45)cc3)c3ccc4c5ccccc5c5ccccc5c4c3)ccc2-c2ccc(N(c3ccccc3)c3cccc4ccccc34)cc21 ; Cc1cc(-c2cccc3c2oc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C]

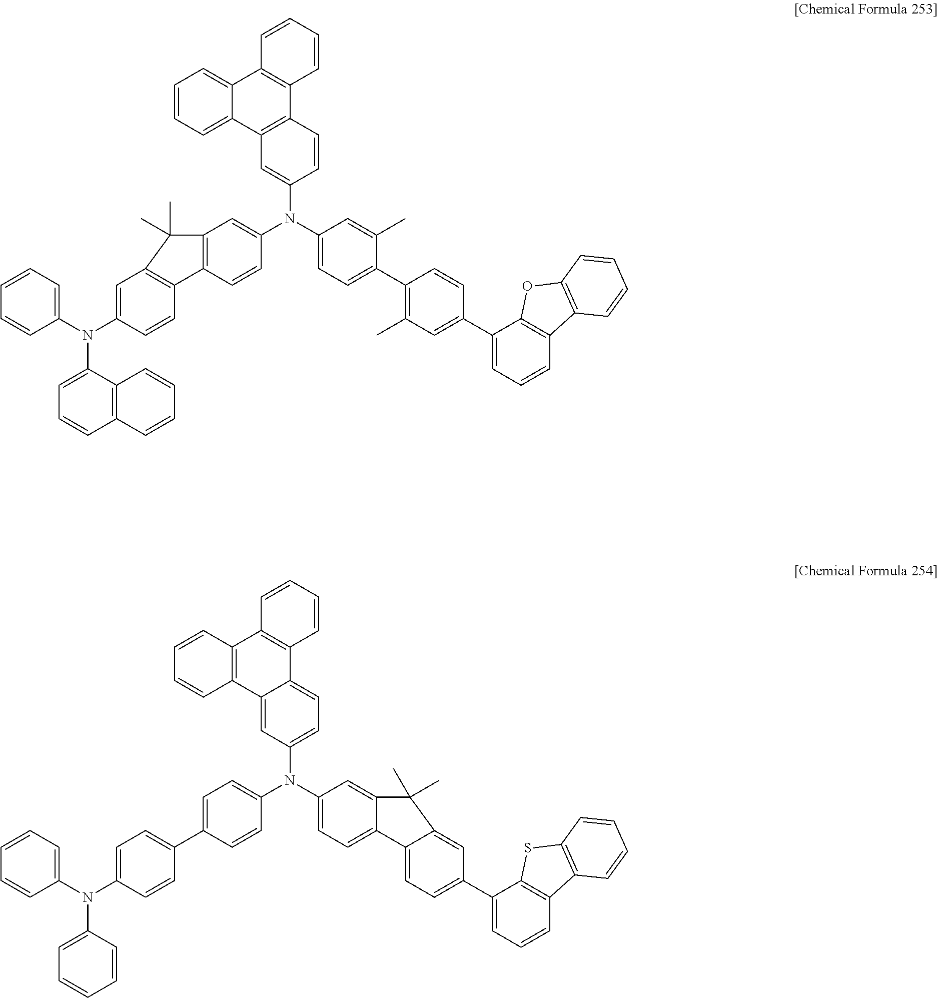 [SMILES: Cc1cc(-c2cccc3c2oc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C ; CC1(C)c2cc(-c3cccc4c3sc3ccccc34)ccc2-c2ccc(N(c3ccc(-c4ccc(N(c5ccccc5)c5ccccc5)cc4)cc3)c3ccc4c5ccccc5c5ccccc5c4c3)cc21]

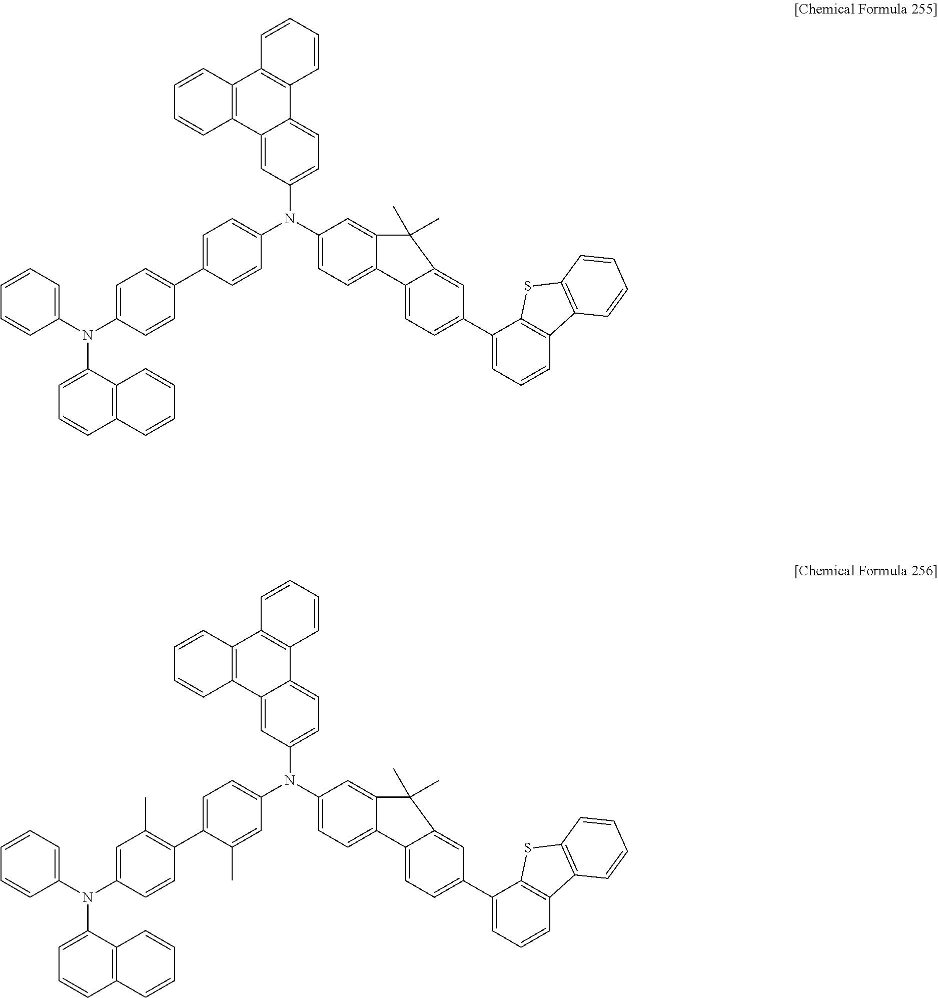 [SMILES: CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21 ; Cc1cc(N(c2ccc3c(c2)C(C)(C)c2cc(-c4cccc5c4sc4ccccc45)ccc2-3)c2ccc3c4ccccc4c4ccccc4c3c2)ccc1-c1ccc(N(c2ccccc2)c2cccc3ccccc23)cc1C]

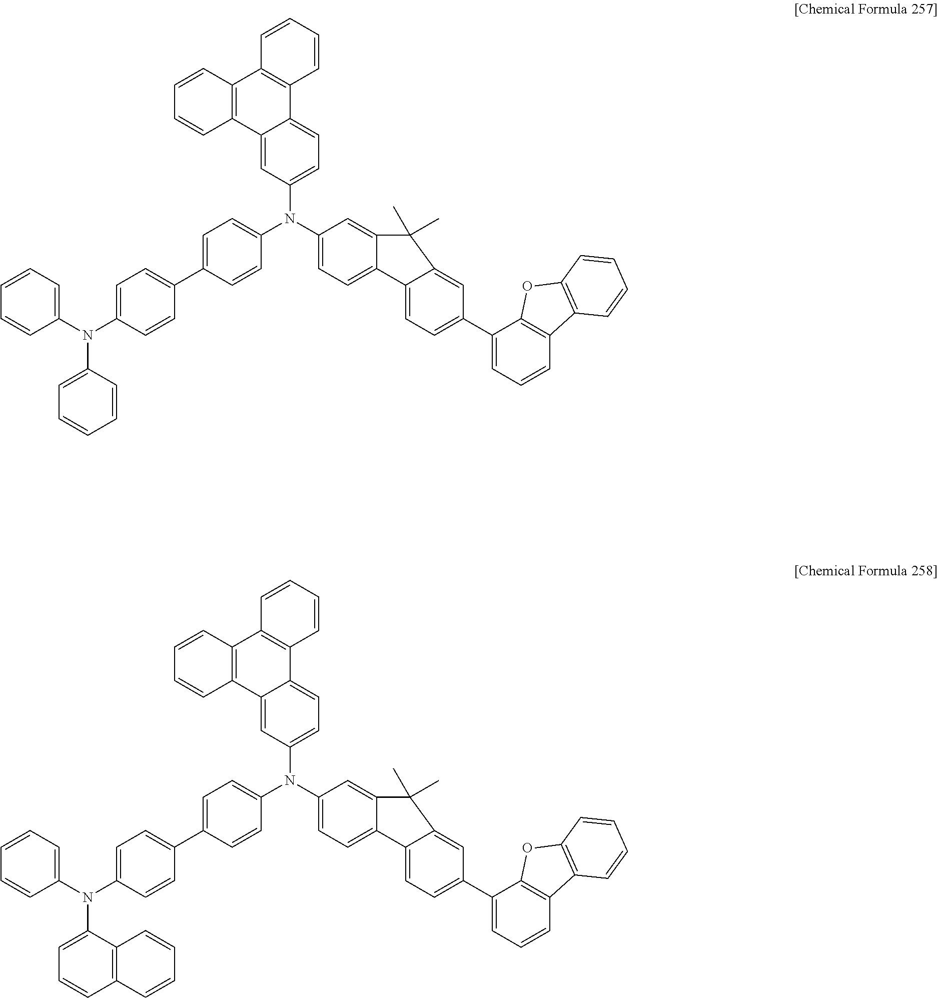 [SMILES: CC1(C)c2cc(-c3cccc4c3oc3ccccc34)ccc2-c2ccc(N(c3ccc(-c4ccc(N(c5ccccc5)c5ccccc5)cc4)cc3)c3ccc4c5ccccc5c5ccccc5c4c3)cc21 ; CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21]

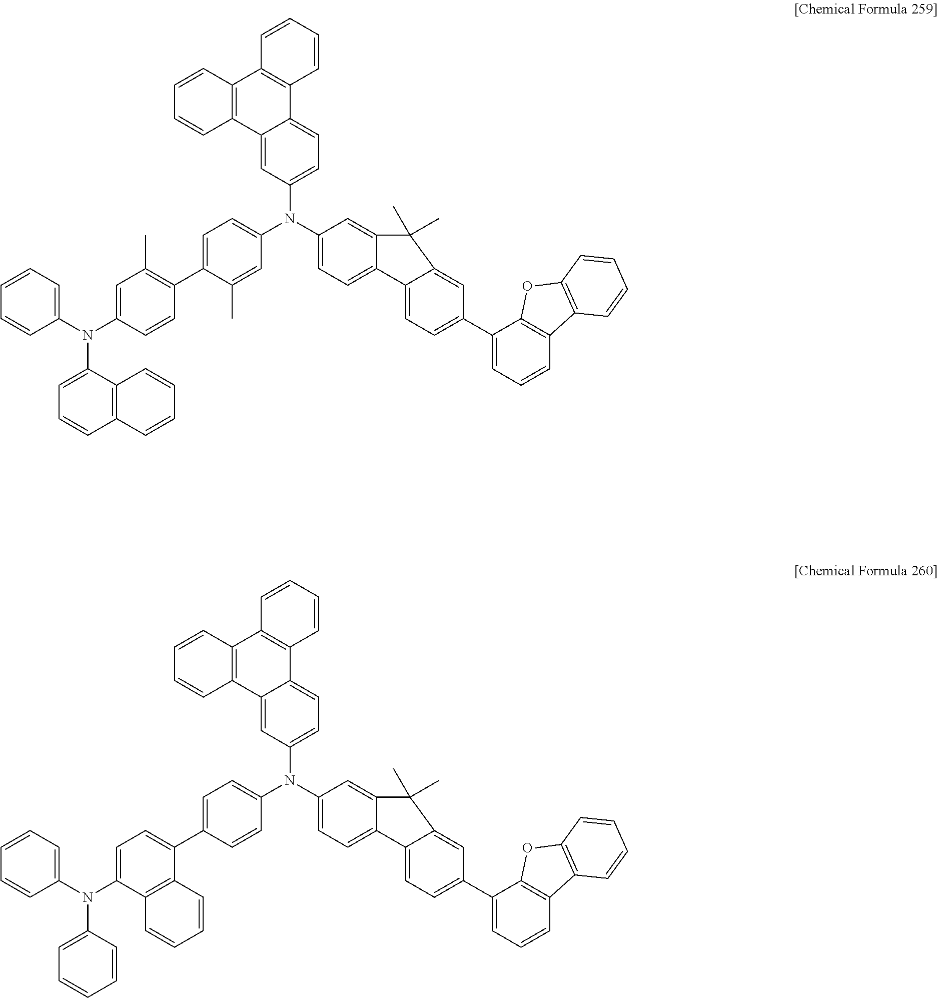 [SMILES: Cc1cc(N(c2ccc3c(c2)C(C)(C)c2cc(-c4cccc5c4oc4ccccc45)ccc2-3)c2ccc3c4ccccc4c4ccccc4c3c2)ccc1-c1ccc(N(c2ccccc2)c2cccc3ccccc23)cc1C ; CC1(C)c2cc(-c3cccc4c3oc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21]

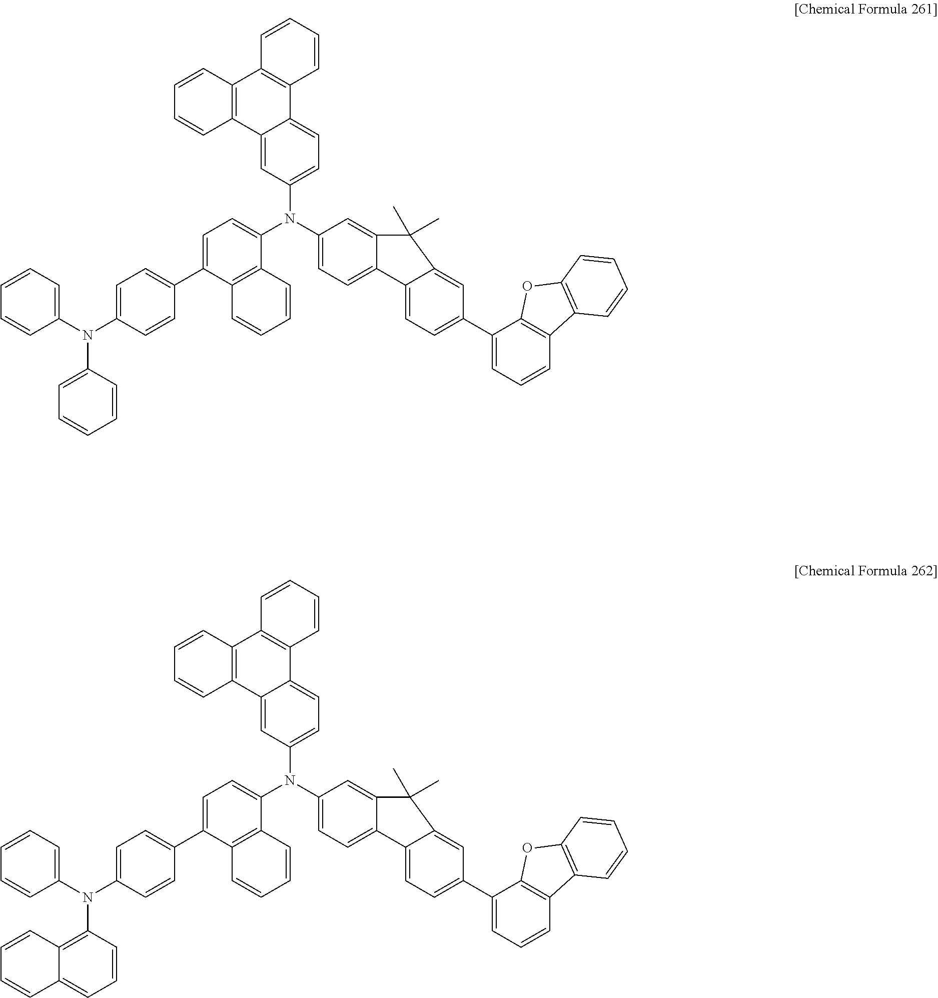 [SMILES: CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21 ; CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21]

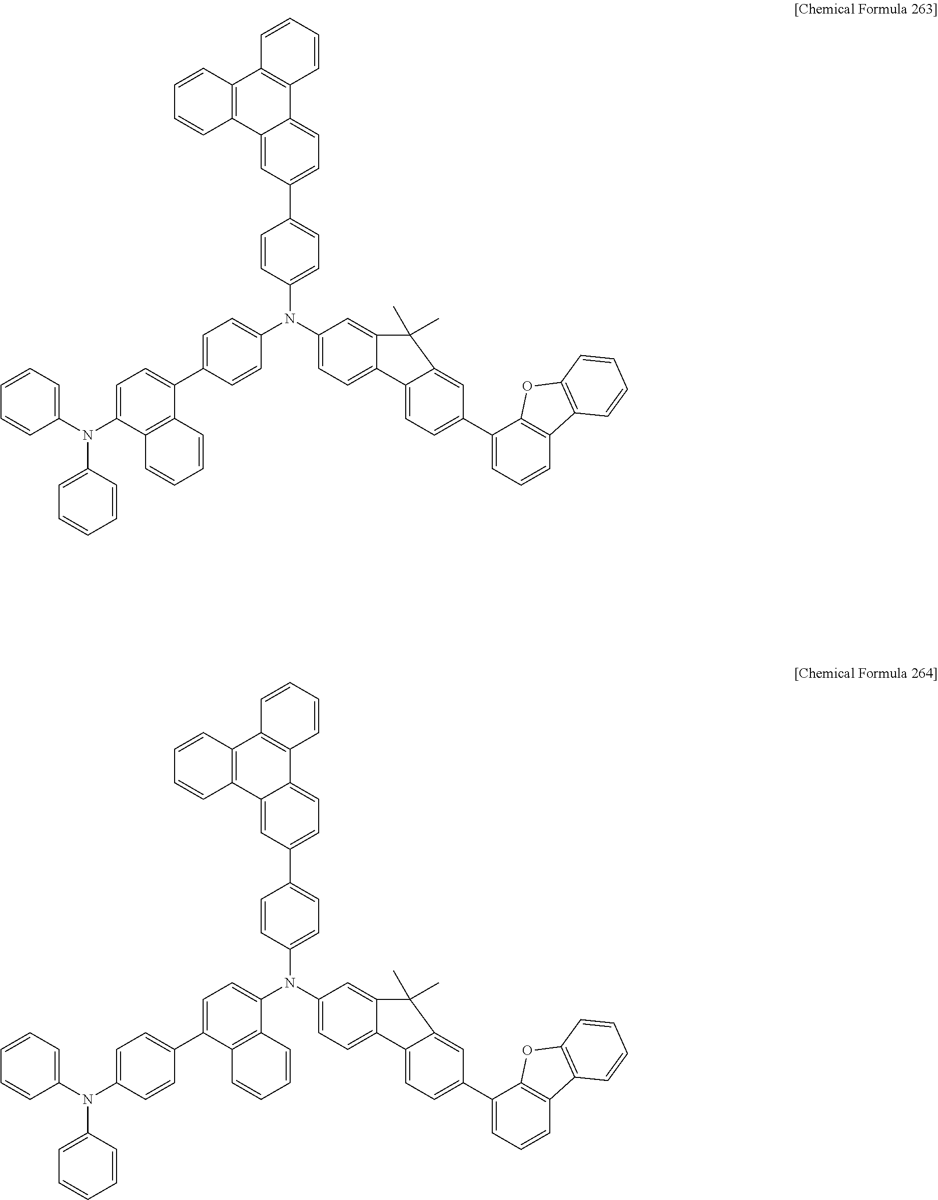 [SMILES: CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21 ; CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21]

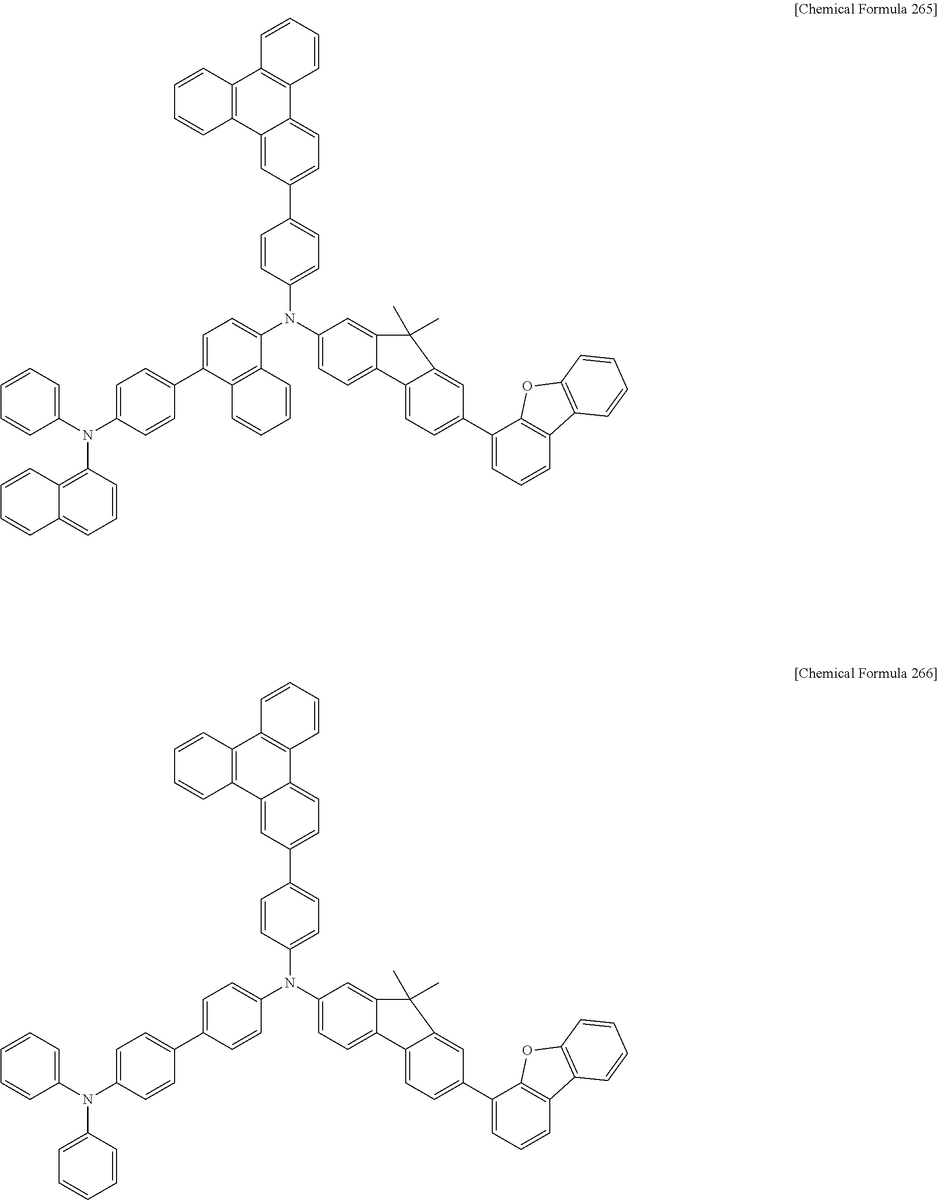 [SMILES: CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21 ; CC1(C)c2cc(-c3cccc4c3oc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21]

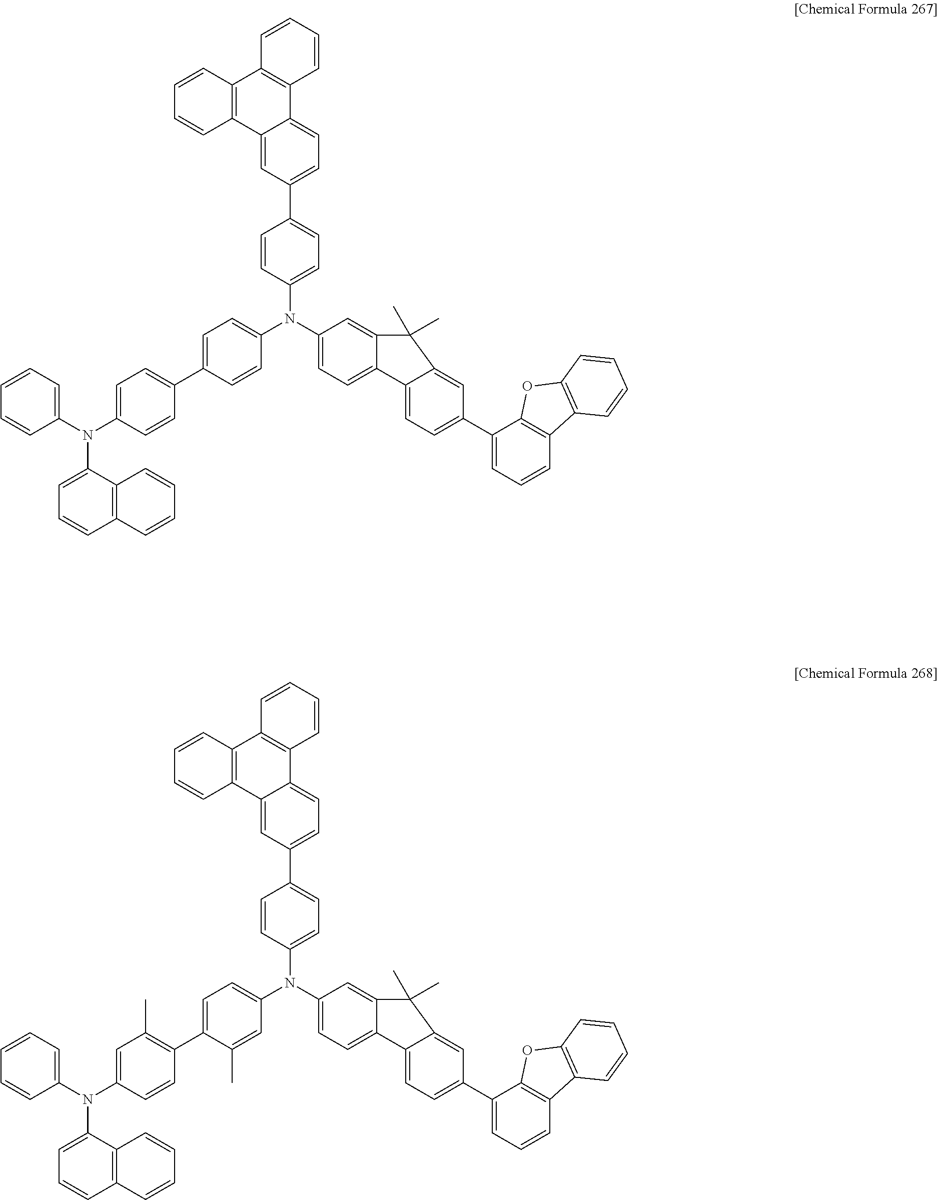 [SMILES: CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21 ; Cc1cc(N(c2ccc(-c3ccc4c5ccccc5c5ccccc5c4c3)cc2)c2ccc3c(c2)C(C)(C)c2cc(-c4cccc5c4oc4ccccc45)ccc2-3)ccc1-c1ccc(N(c2ccccc2)c2cccc3ccccc23)cc1C]

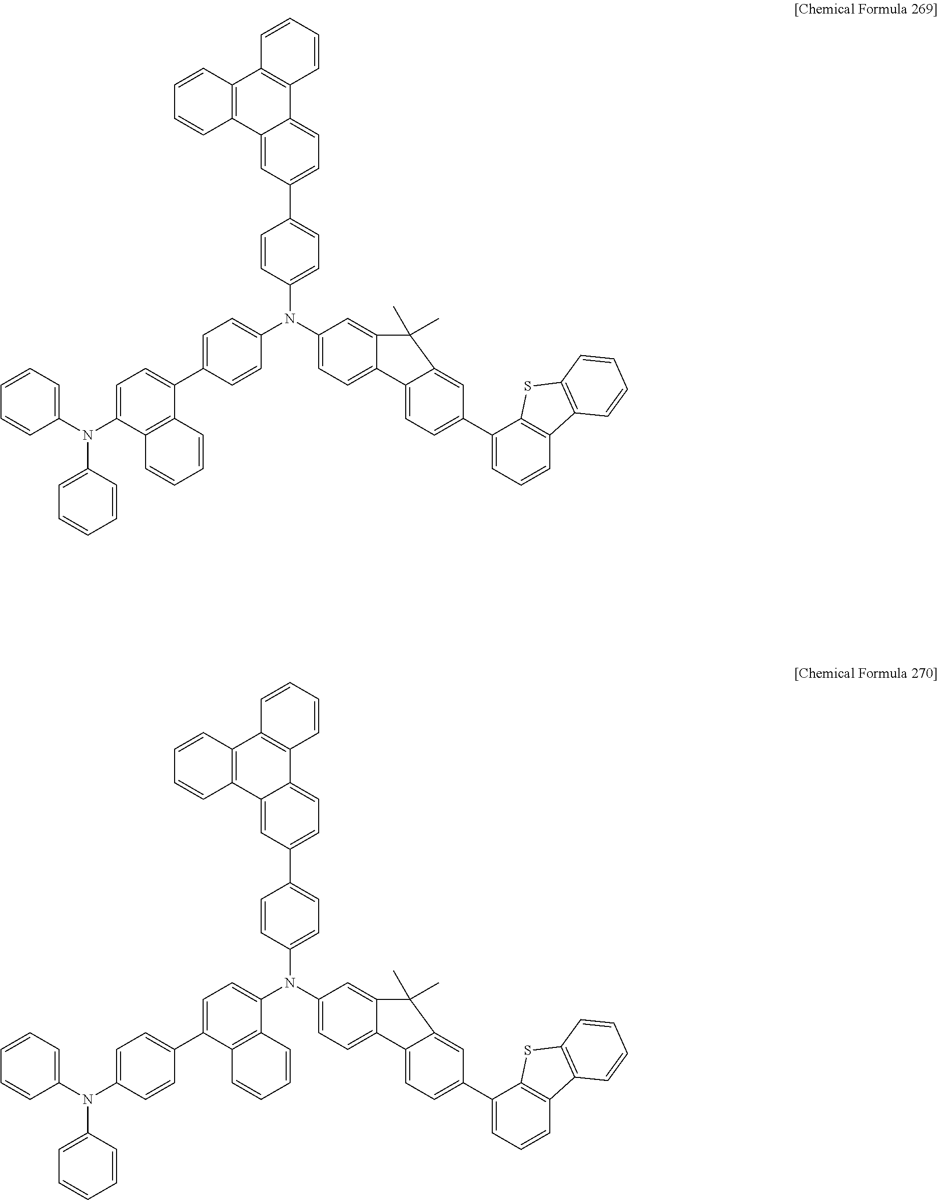 [SMILES: CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21 ; CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21]

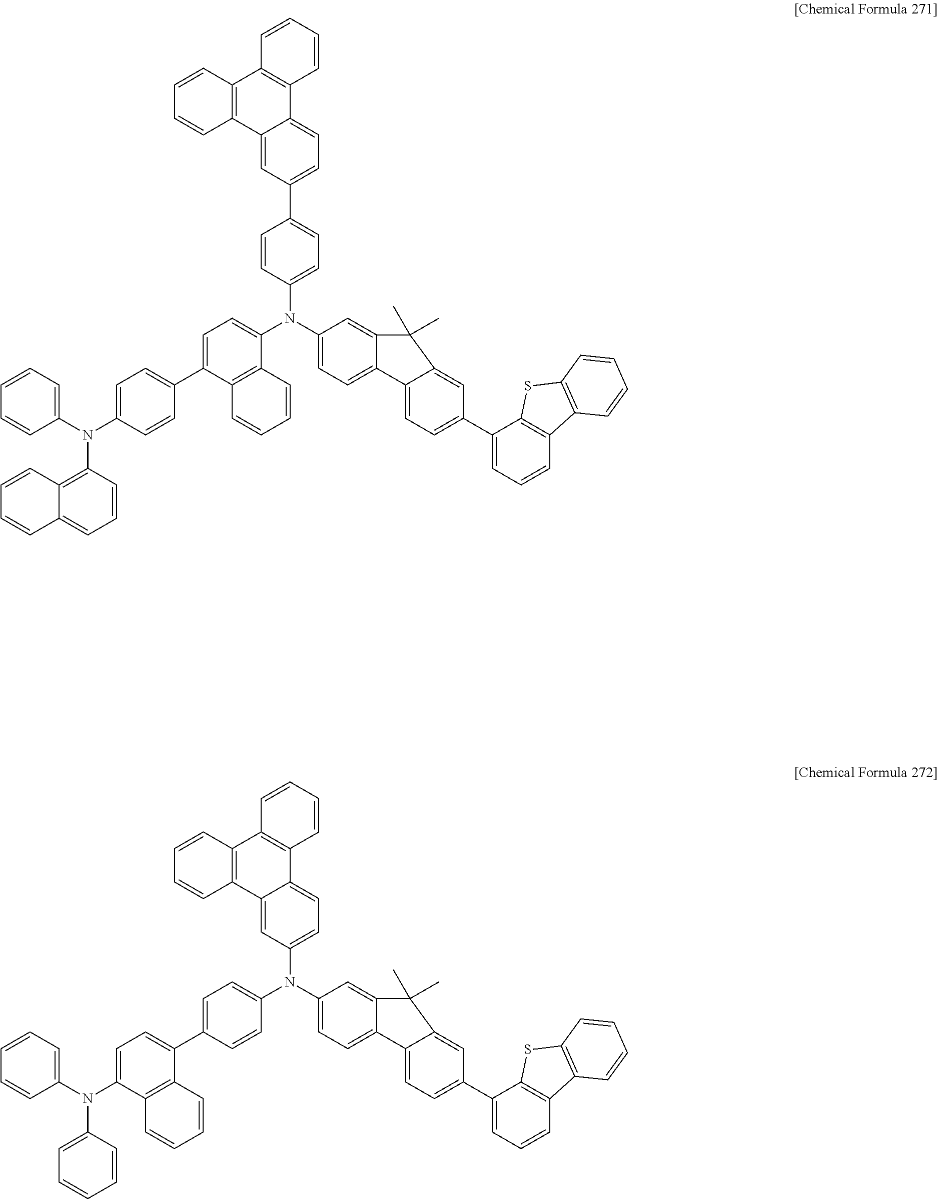 [SMILES: CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21 ; CC1(C)c2cc(-c3cccc4c3sc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21]

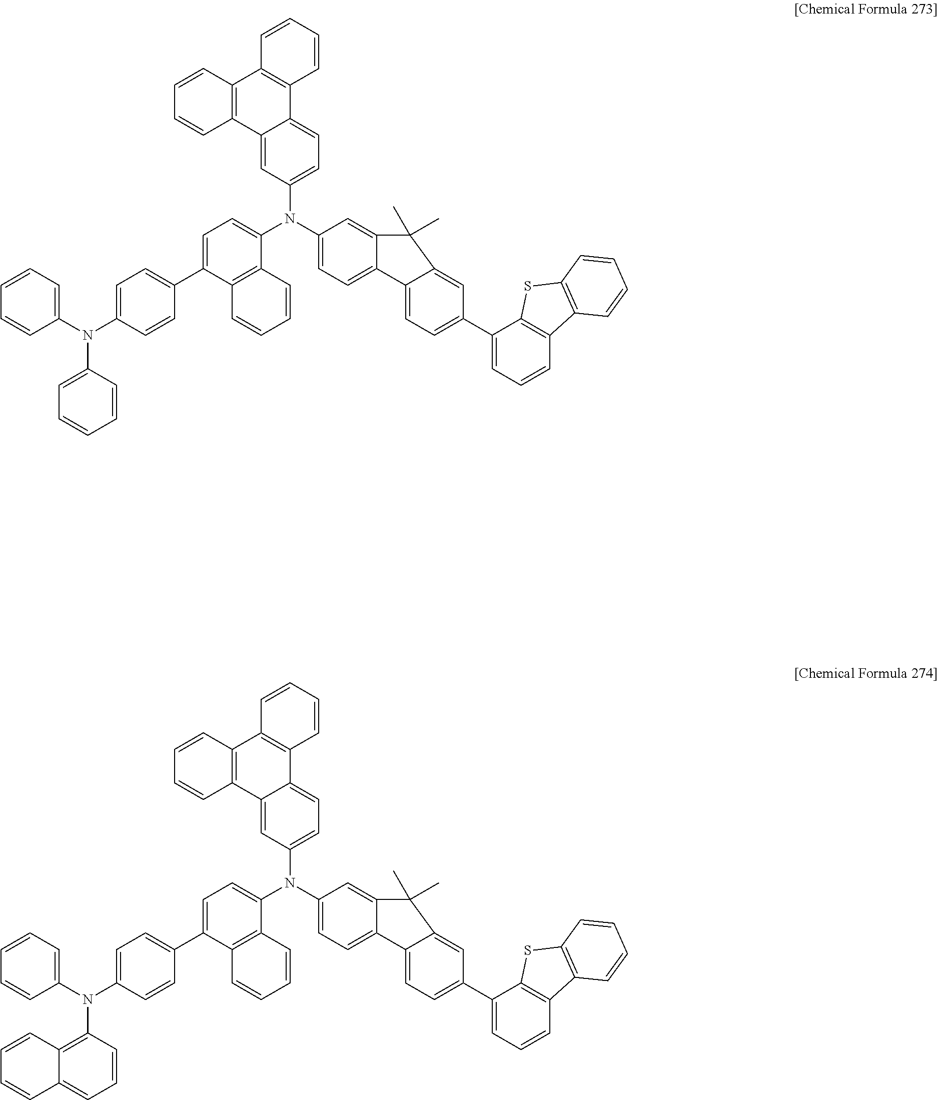 [SMILES: CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21 ; CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21]

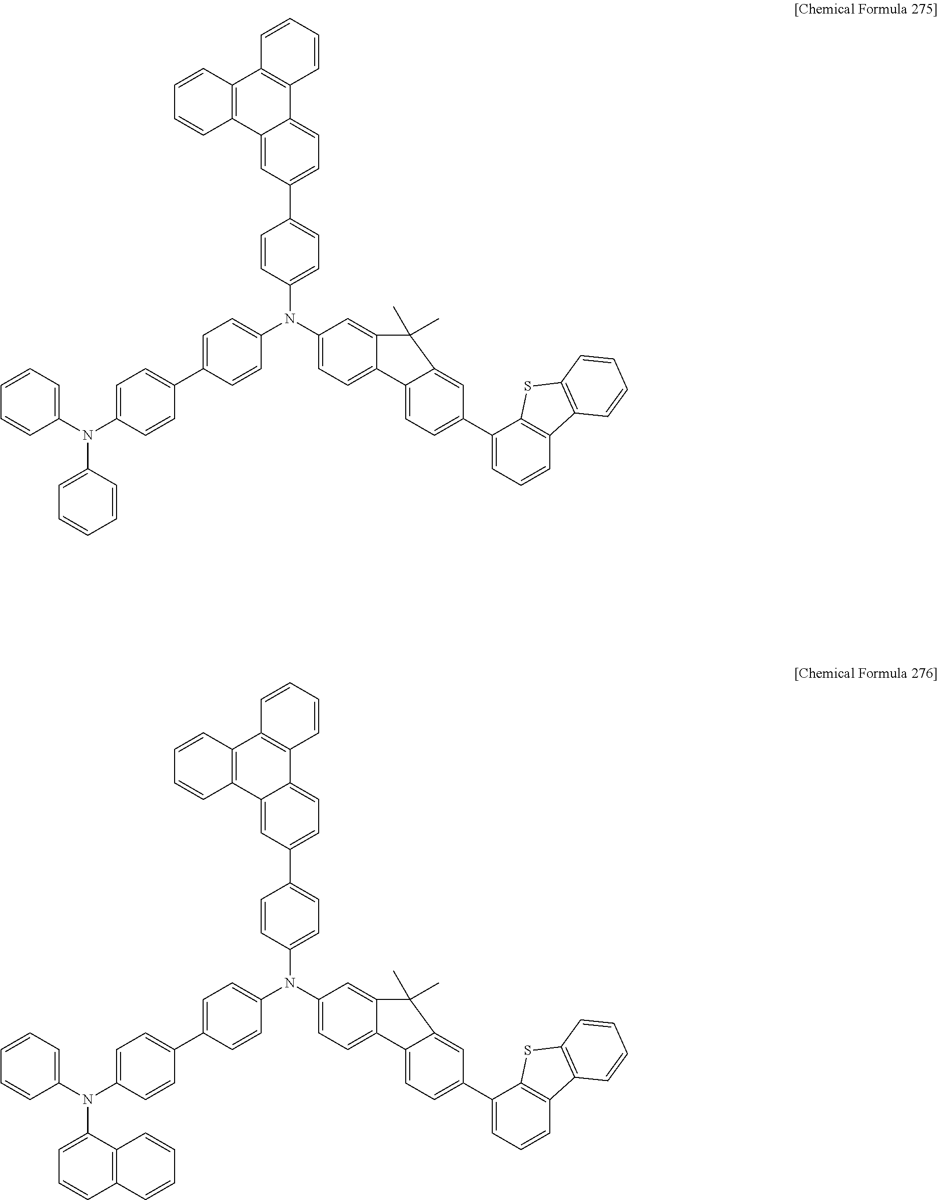 [SMILES: CC1(C)c2cc(-c3cccc4c3sc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21 ; CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21]

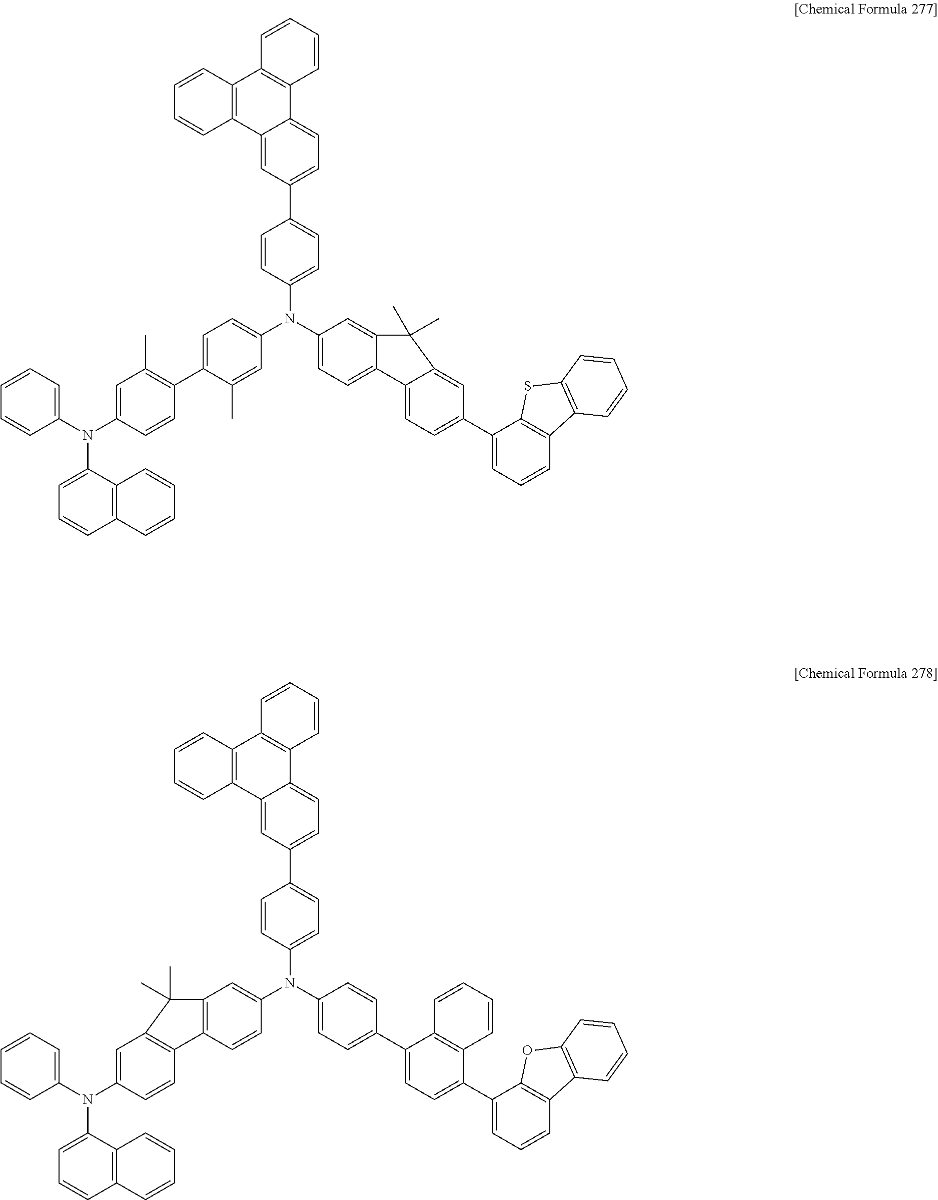 [SMILES: Cc1cc(N(c2ccc(-c3ccc4c5ccccc5c5ccccc5c4c3)cc2)c2ccc3c(c2)C(C)(C)c2cc(-c4cccc5c4sc4ccccc45)ccc2-3)ccc1-c1ccc(N(c2ccccc2)c2cccc3ccccc23)cc1C ; CC1(C)c2cc(N(c3ccc(-c4ccc5c6ccccc6c6ccccc6c5c4)cc3)c3ccc(-c4ccc(-c5cccc6c5oc5ccccc56)c5ccccc45)cc3)ccc2-c2ccc(N(c3ccccc3)c3cccc4ccccc34)cc21]

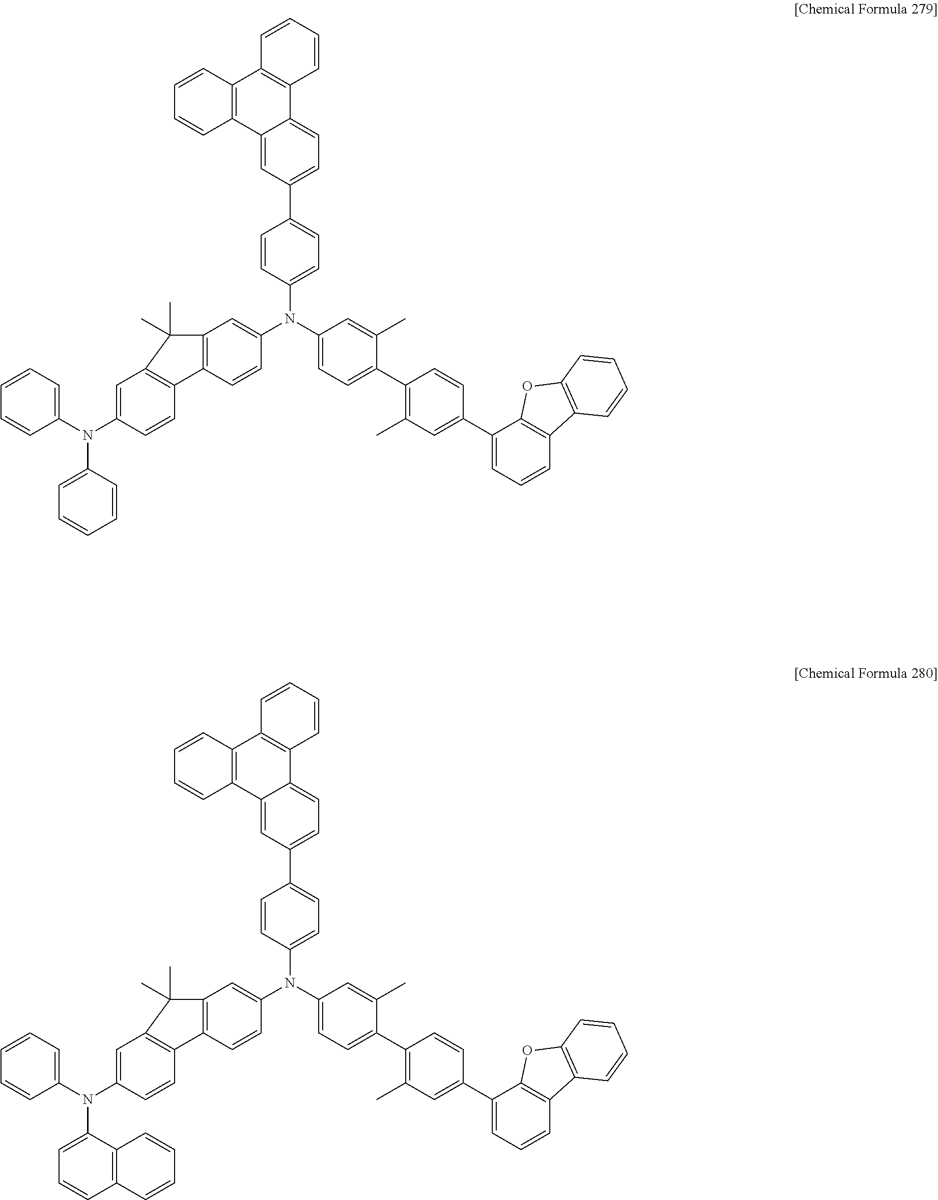 [SMILES: Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C]

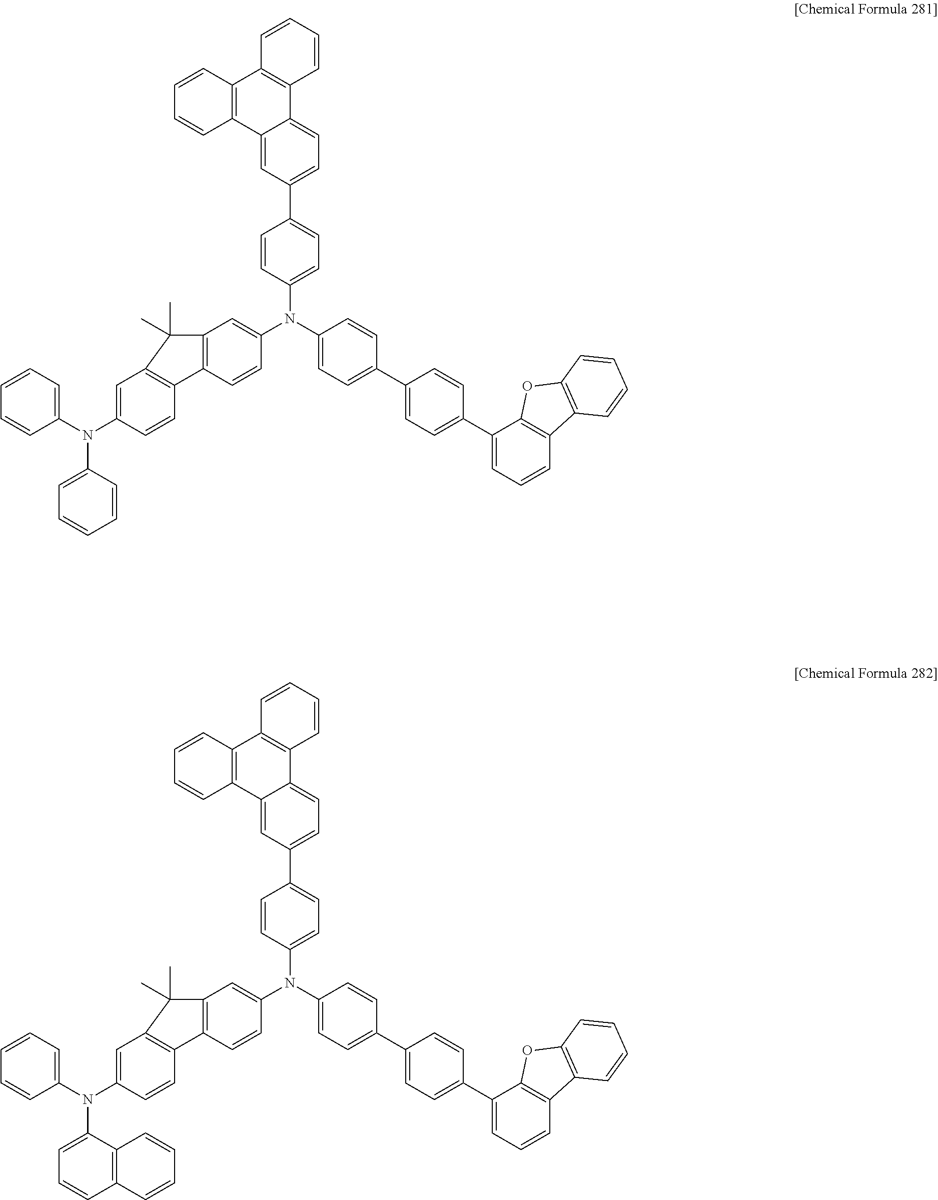 [SMILES: CC1(C)c2cc(N(c3ccccc3)c3ccccc3)ccc2-c2ccc(N(c3ccc(-c4ccc(-c5cccc6c5oc5ccccc56)cc4)cc3)c3ccc(-c4ccc5c6ccccc6c6ccccc6c5c4)cc3)cc21 ; CC1(C)c2cc(N(c3ccc(-c4ccc(-c5cccc6c5oc5ccccc56)cc4)cc3)c3ccc(-c4ccc5c6ccccc6c6ccccc6c5c4)cc3)ccc2-c2ccc(N(c3ccccc3)c3cccc4ccccc34)cc21]

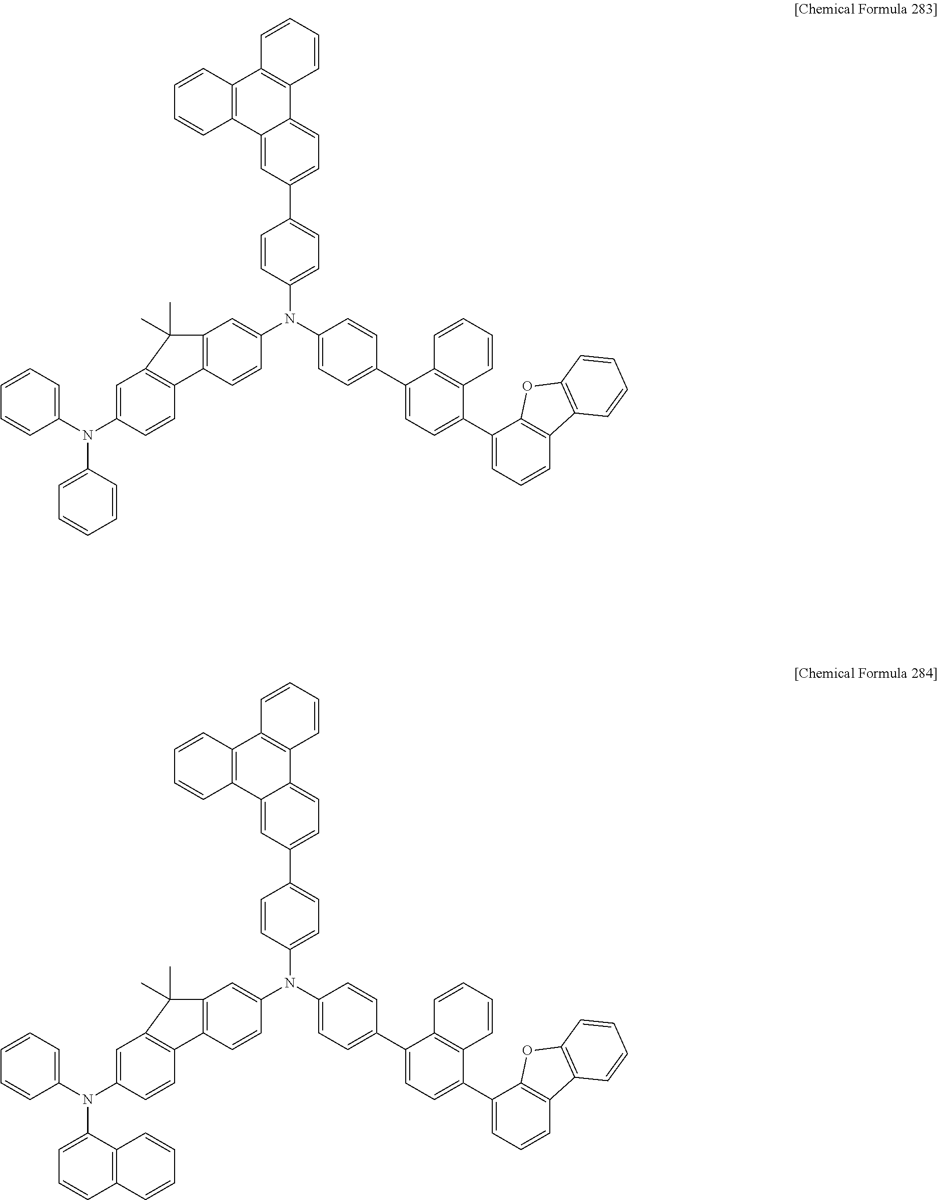 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5oc5ccccc56)c5ccccc45)cc3)cc21 ; CC1(C)c2cc(N(c3ccc(-c4ccc5c6ccccc6c6ccccc6c5c4)cc3)c3ccc(-c4ccc(-c5cccc6c5oc5ccccc56)c5ccccc45)cc3)ccc2-c2ccc(N(c3ccccc3)c3cccc4ccccc34)cc21]

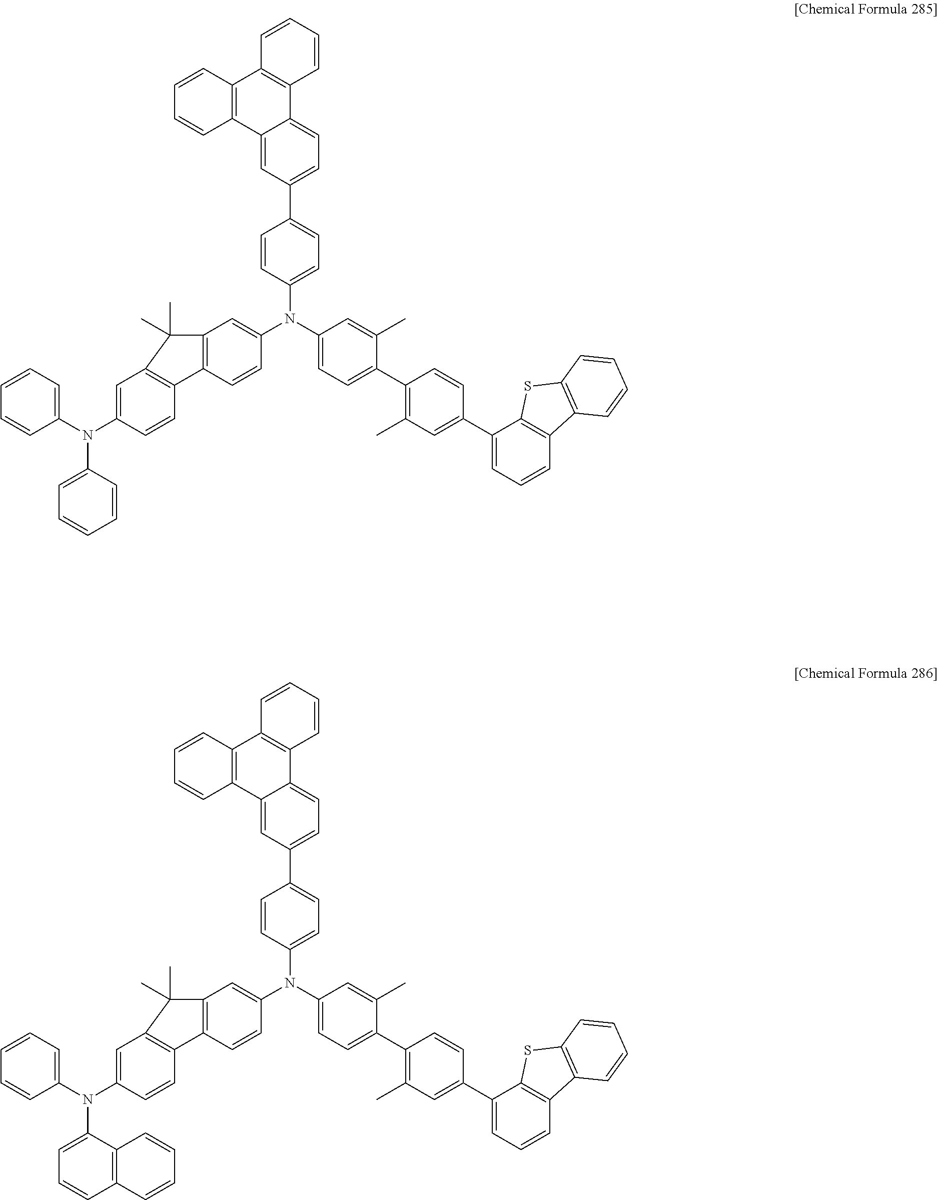 [SMILES: Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C]

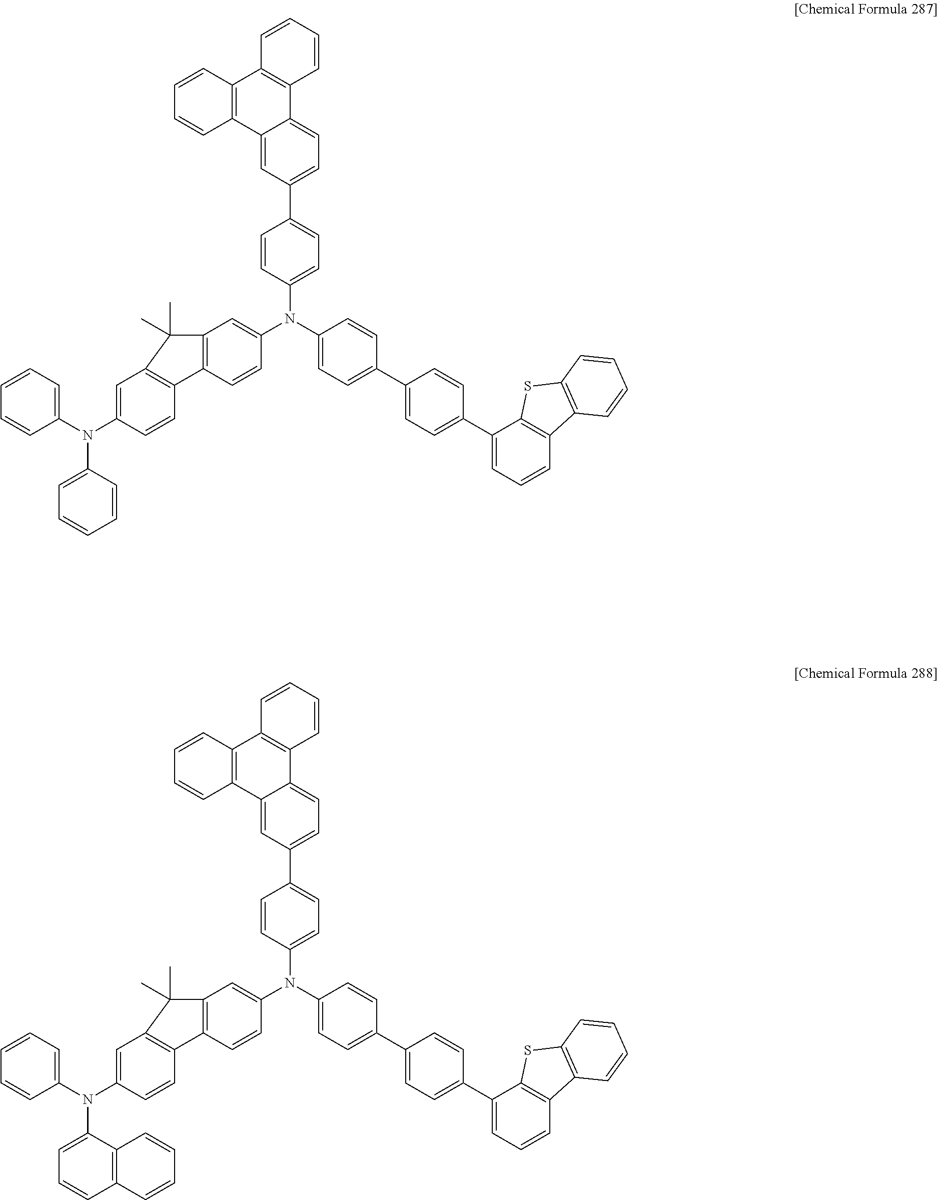 [SMILES: CC1(C)c2cc(N(c3ccccc3)c3ccccc3)ccc2-c2ccc(N(c3ccc(-c4ccc(-c5cccc6c5sc5ccccc56)cc4)cc3)c3ccc(-c4ccc5c6ccccc6c6ccccc6c5c4)cc3)cc21 ; CC1(C)c2cc(N(c3ccc(-c4ccc(-c5cccc6c5sc5ccccc56)cc4)cc3)c3ccc(-c4ccc5c6ccccc6c6ccccc6c5c4)cc3)ccc2-c2ccc(N(c3ccccc3)c3cccc4ccccc34)cc21]

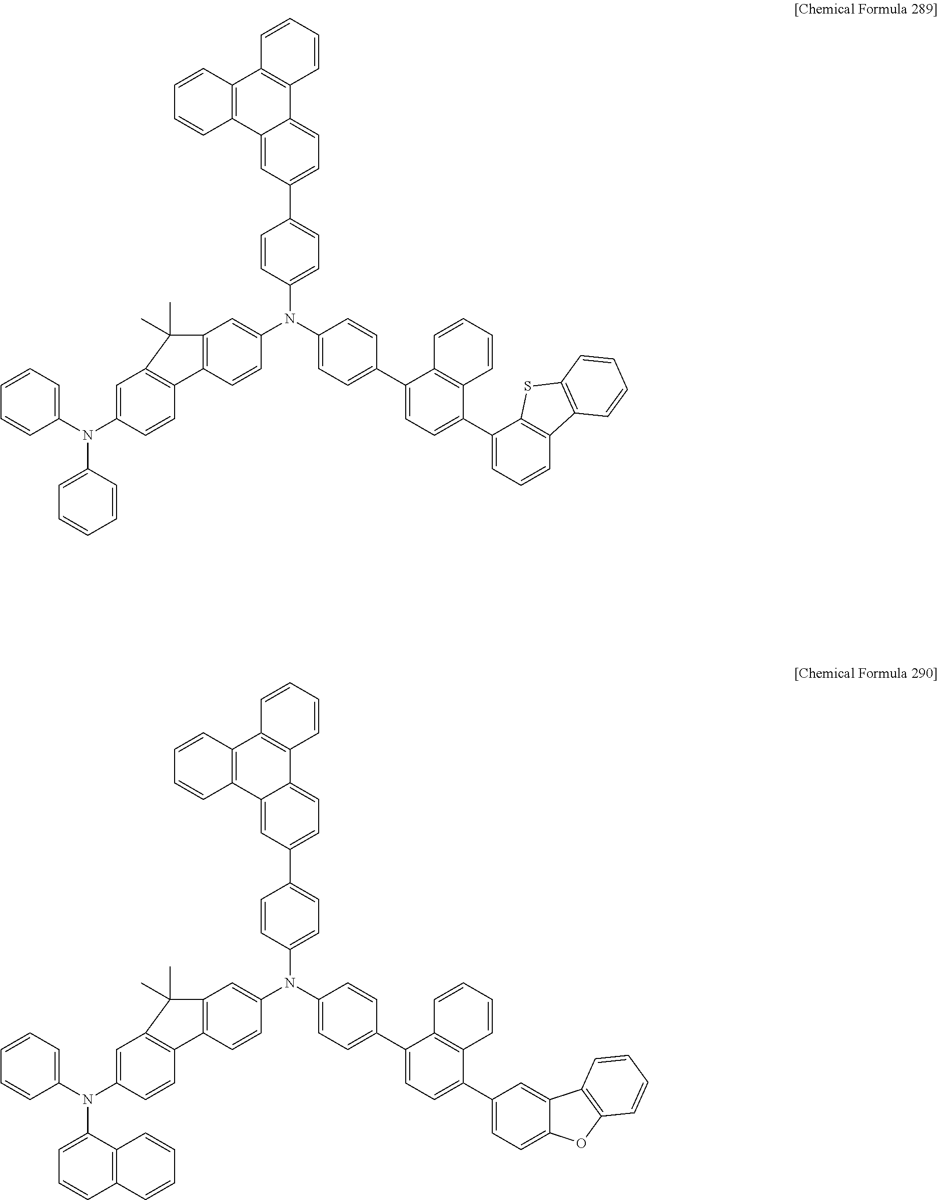 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5sc5ccccc56)c5ccccc45)cc3)cc21 ; CC1(C)c2cc(N(c3ccc(-c4ccc5c6ccccc6c6ccccc6c5c4)cc3)c3ccc(-c4ccc(-c5ccc6oc7ccccc7c6c5)c5ccccc45)cc3)ccc2-c2ccc(N(c3ccccc3)c3cccc4ccccc34)cc21]

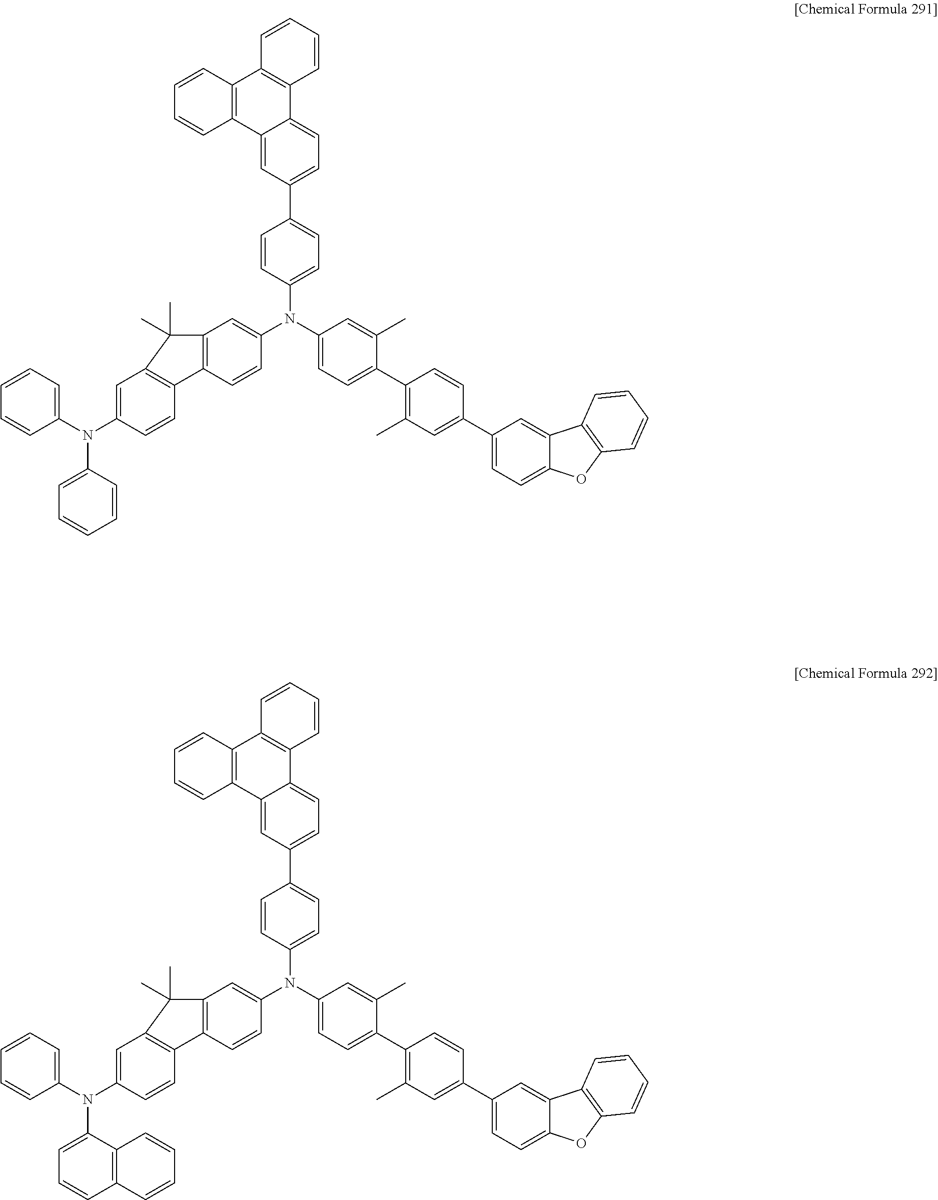 [SMILES: Cc1cc(-c2ccc3oc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2ccc3oc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C]

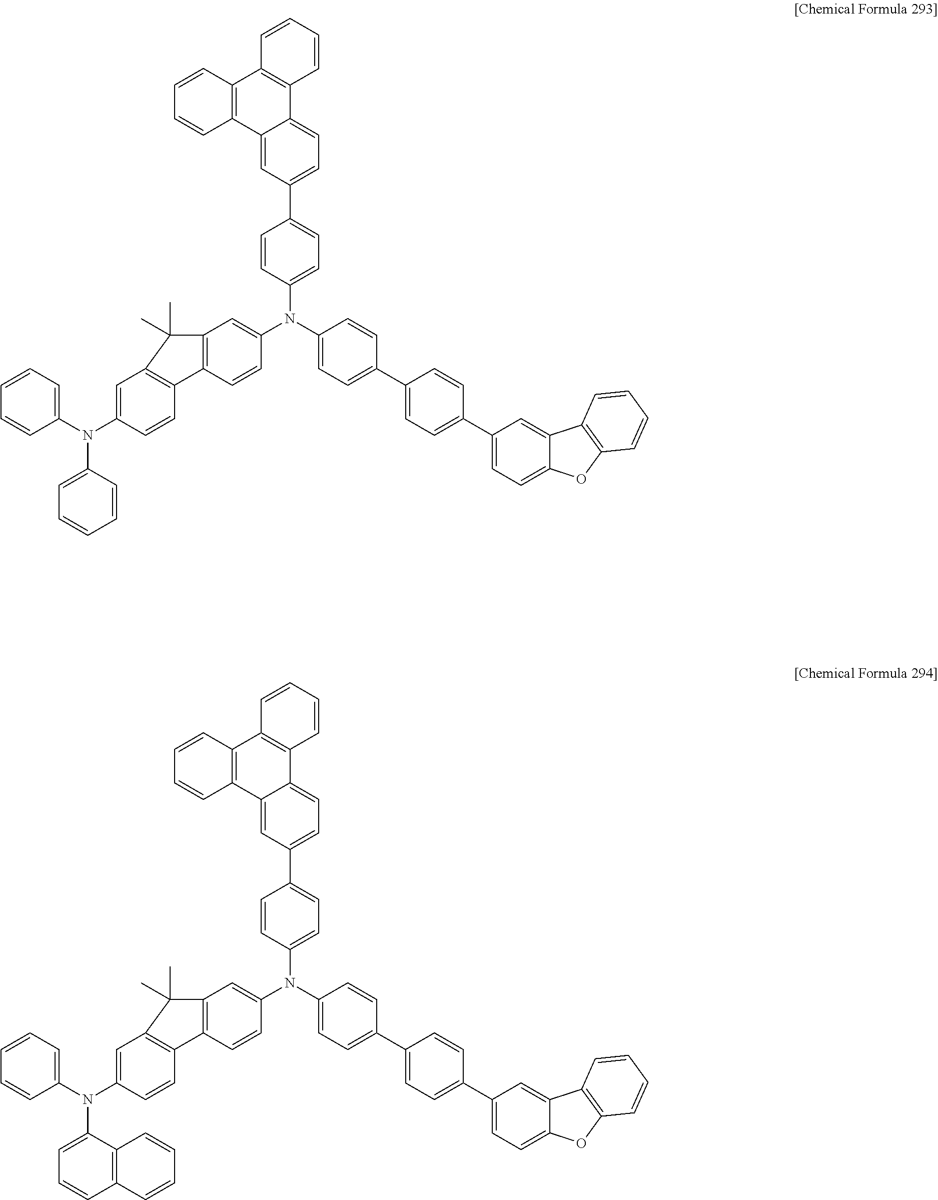 [SMILES: CC1(C)c2cc(N(c3ccccc3)c3ccccc3)ccc2-c2ccc(N(c3ccc(-c4ccc(-c5ccc6oc7ccccc7c6c5)cc4)cc3)c3ccc(-c4ccc5c6ccccc6c6ccccc6c5c4)cc3)cc21 ; CC1(C)c2cc(N(c3ccc(-c4ccc(-c5ccc6oc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21]

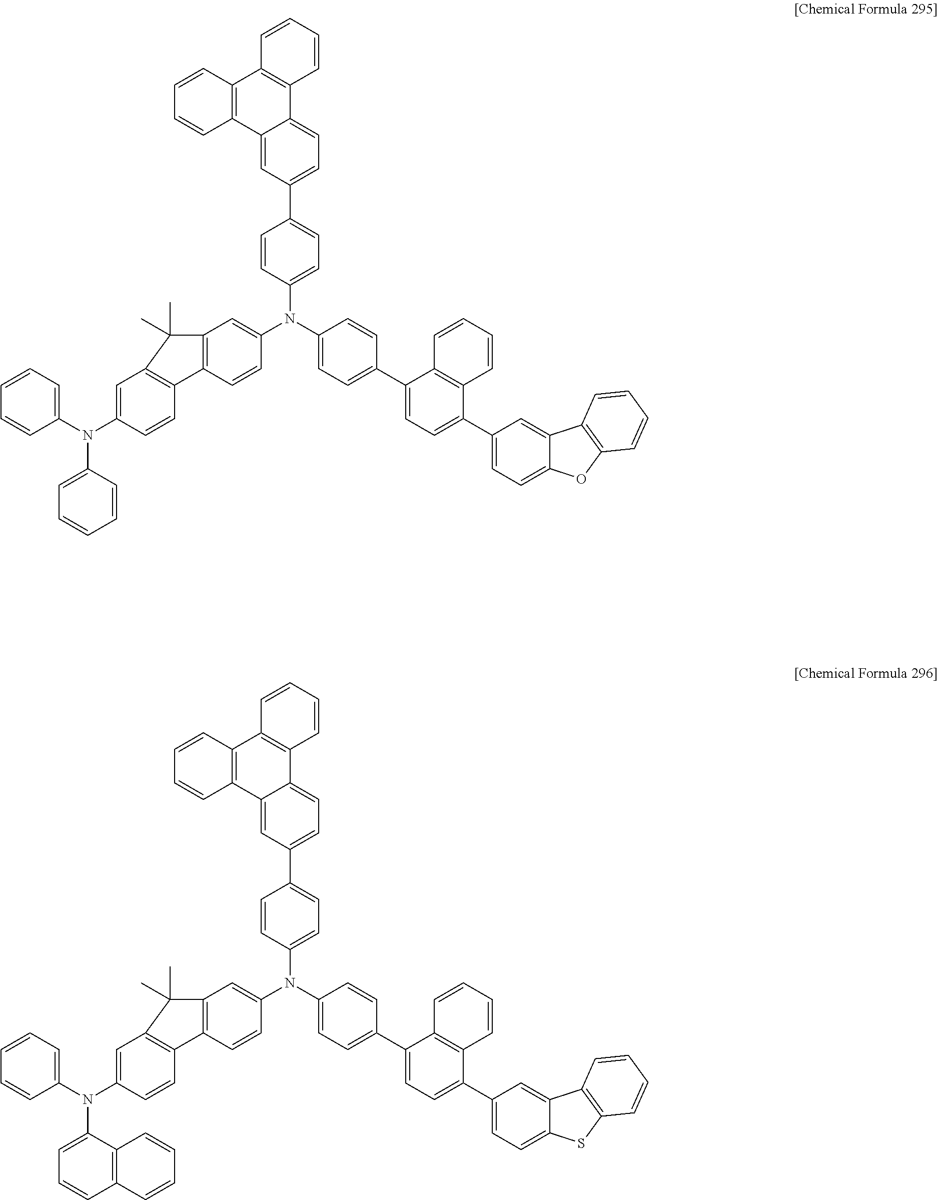 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6oc7ccccc7c6c5)c5ccccc45)cc3)cc21 ; CC1(C)c2cc(N(c3ccc(-c4ccc5c6ccccc6c6ccccc6c5c4)cc3)c3ccc(-c4ccc(-c5ccc6sc7ccccc7c6c5)c5ccccc45)cc3)ccc2-c2ccc(N(c3ccccc3)c3cccc4ccccc34)cc21]

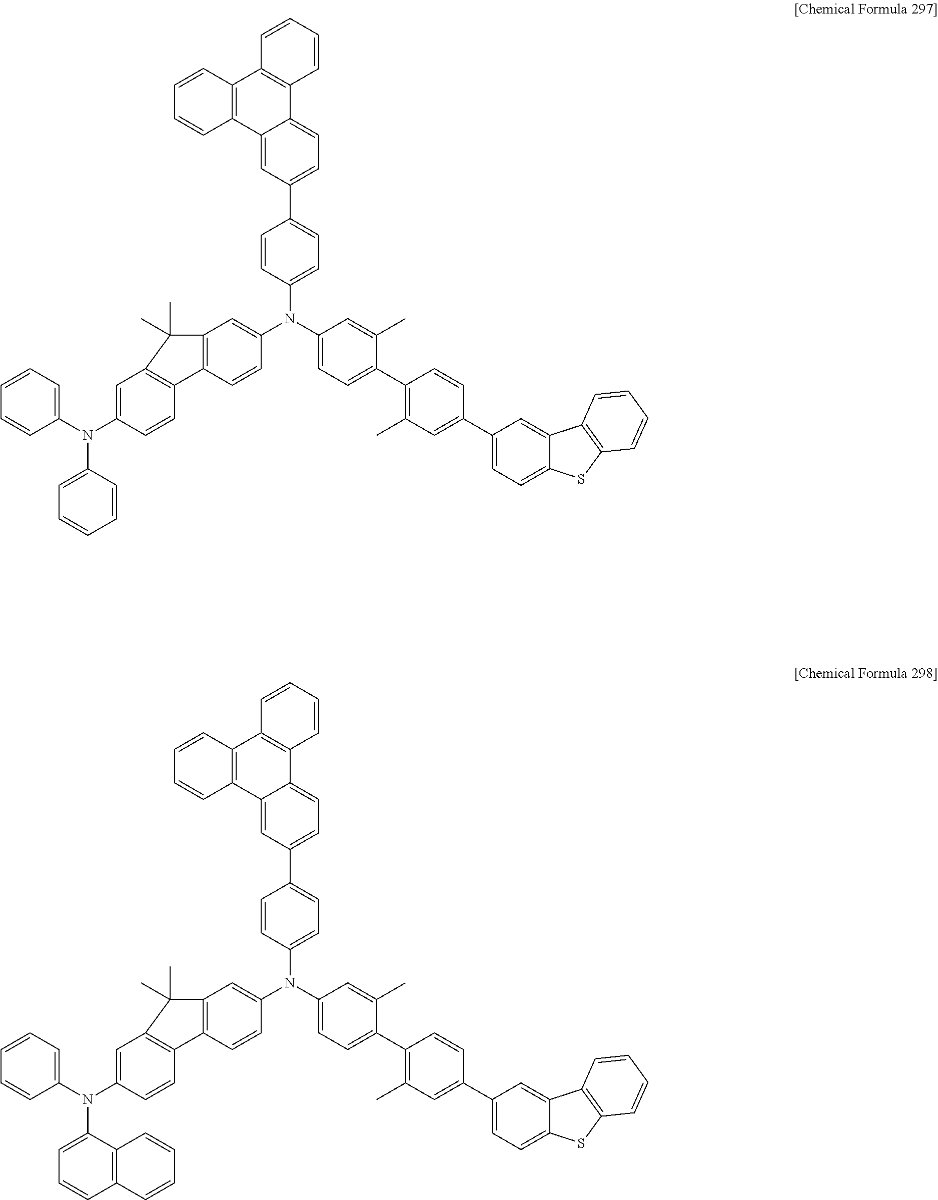 [SMILES: Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C]

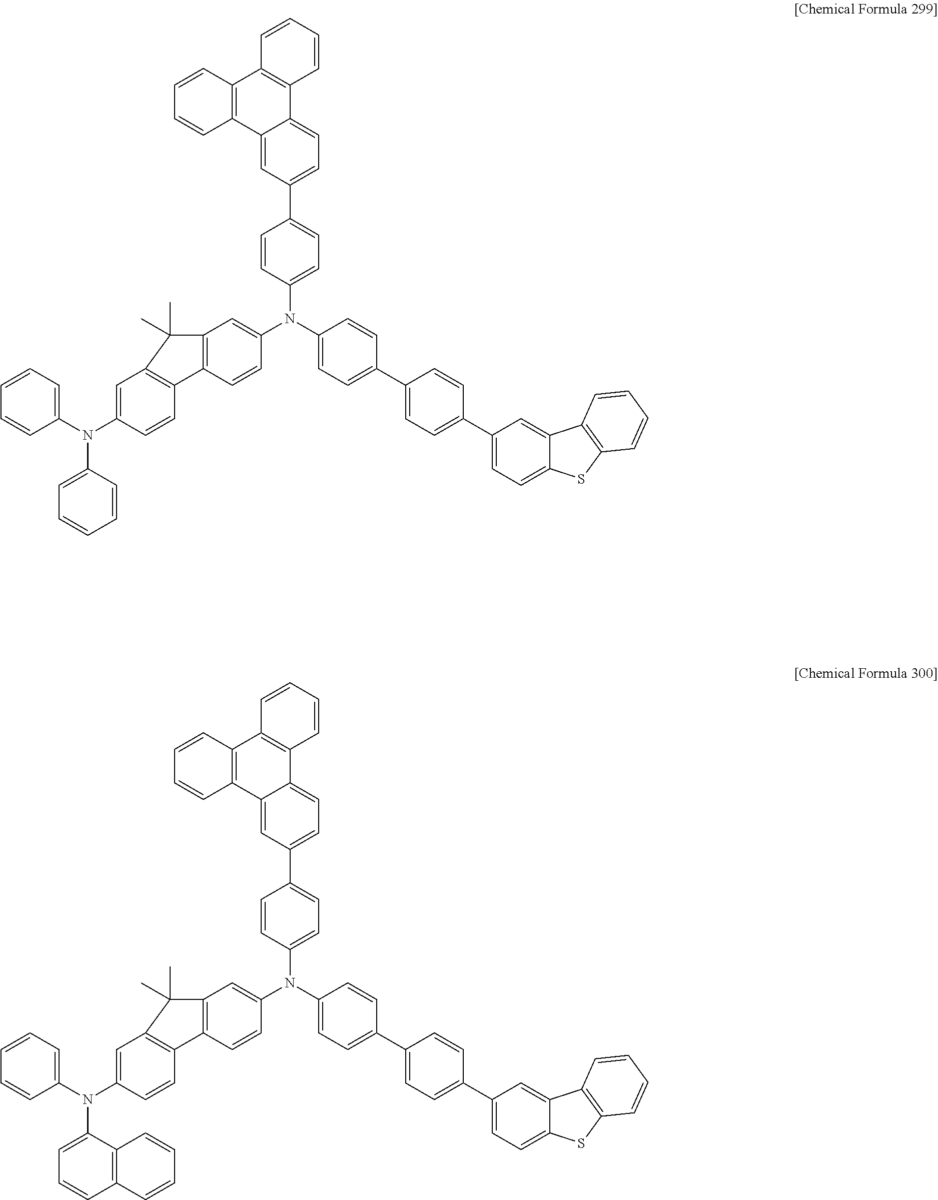 [SMILES: CC1(C)c2cc(N(c3ccccc3)c3ccccc3)ccc2-c2ccc(N(c3ccc(-c4ccc(-c5ccc6sc7ccccc7c6c5)cc4)cc3)c3ccc(-c4ccc5c6ccccc6c6ccccc6c5c4)cc3)cc21 ; CC1(C)c2cc(N(c3ccc(-c4ccc(-c5ccc6sc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21]

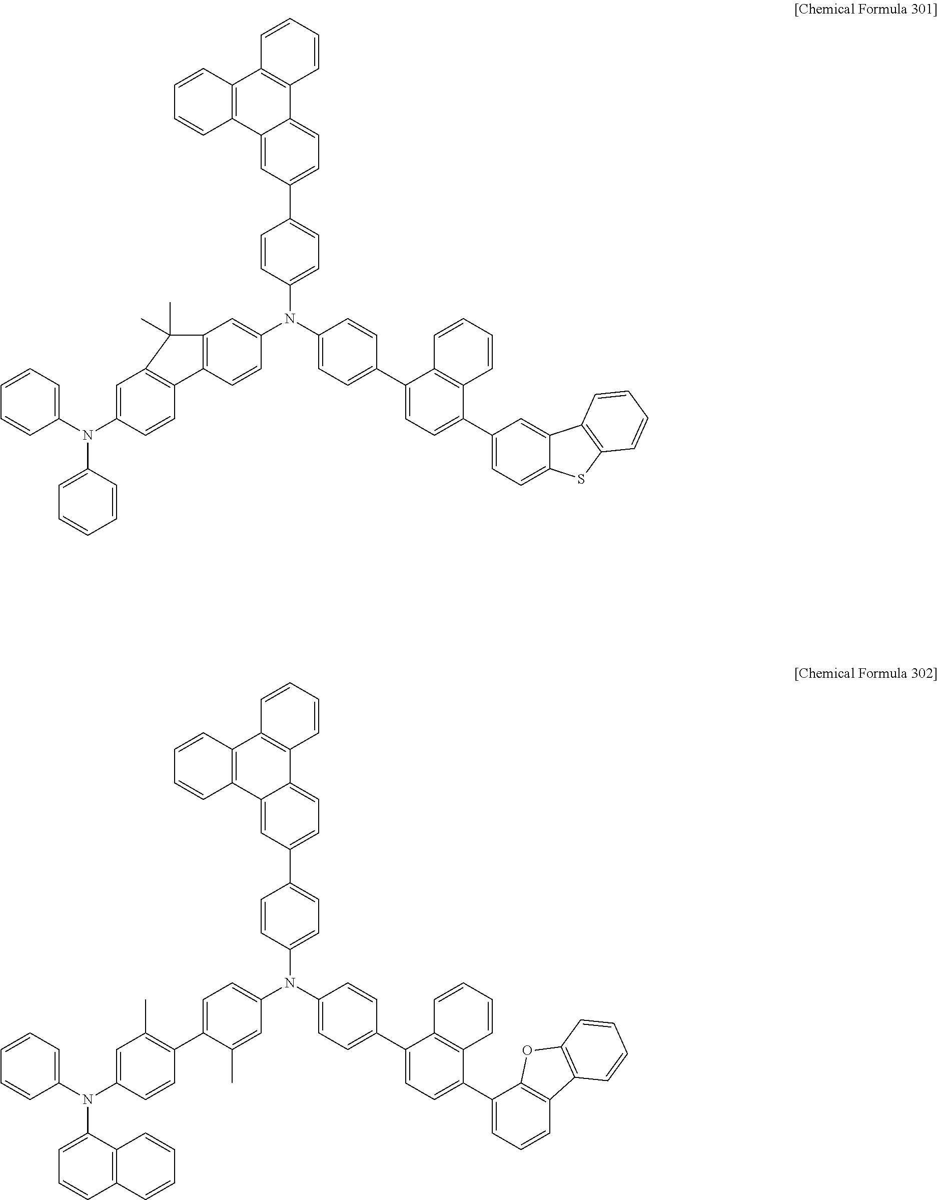 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6sc7ccccc7c6c5)c5ccccc45)cc3)cc21 ; Cc1cc(N(c2ccc(-c3ccc4c5ccccc5c5ccccc5c4c3)cc2)c2ccc(-c3ccc(-c4cccc5c4oc4ccccc45)c4ccccc34)cc2)ccc1-c1ccc(N(c2ccccc2)c2cccc3ccccc23)cc1C]

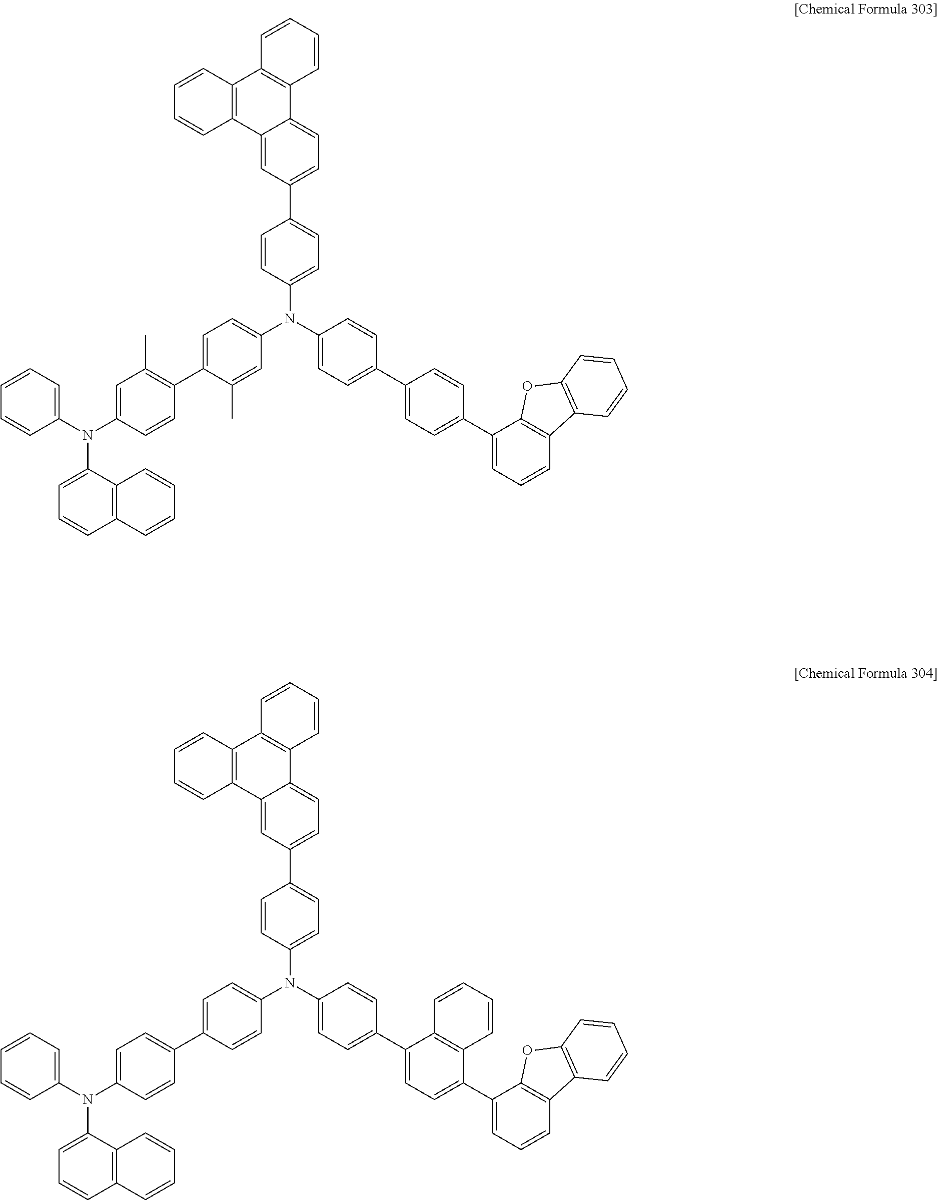 [SMILES: Cc1cc(N(c2ccc(-c3ccc(-c4cccc5c4oc4ccccc45)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1]

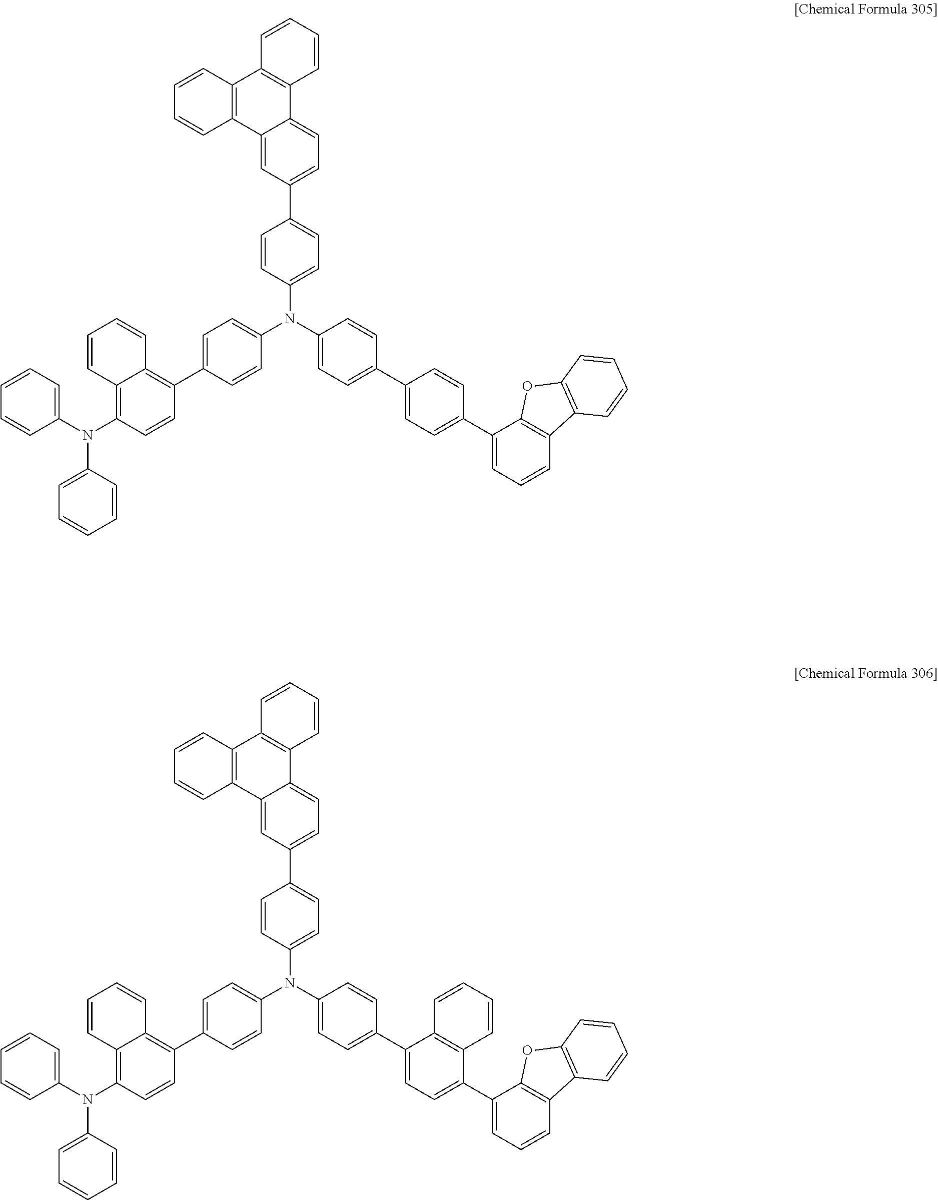 [SMILES: c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)c3ccccc23)cc1]

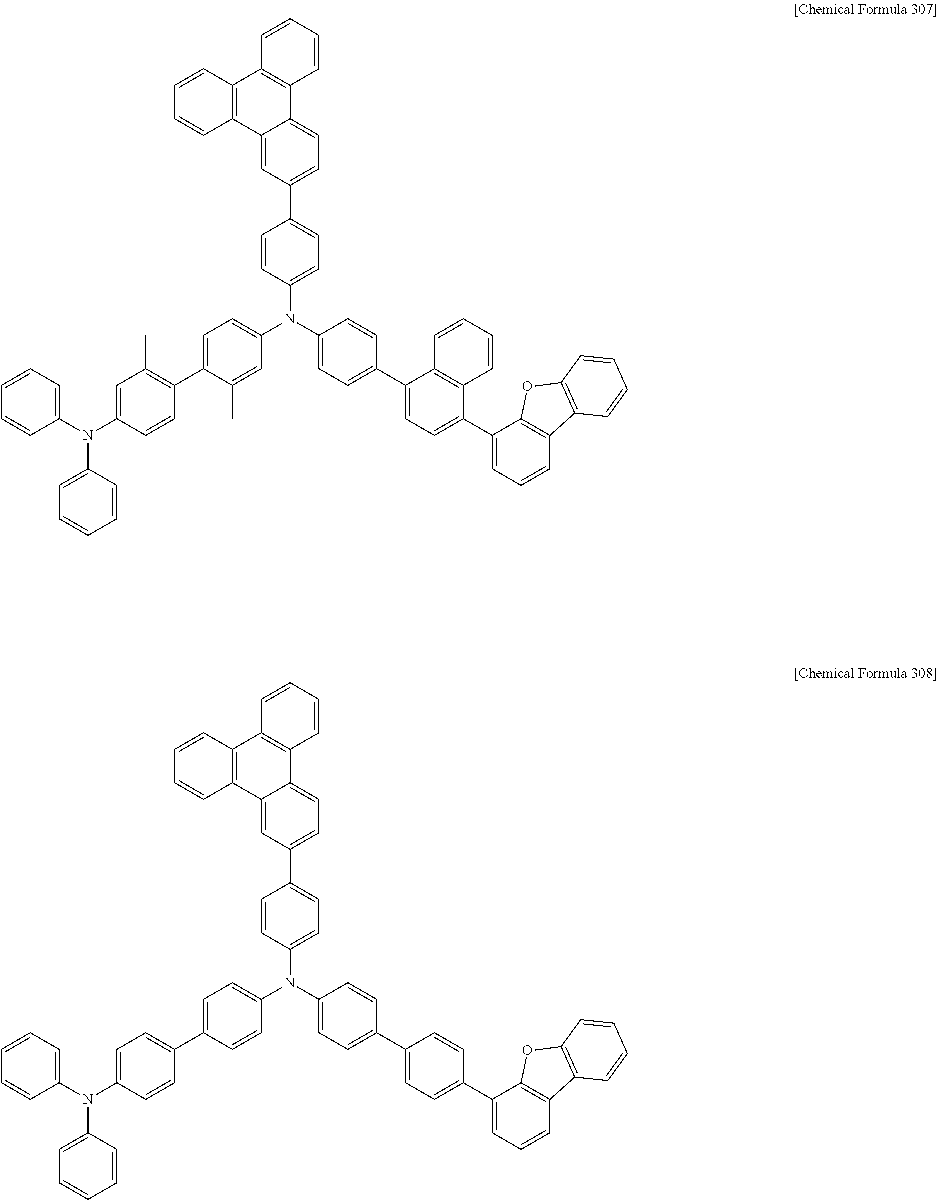 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4oc4ccccc45)c4ccccc34)cc2)cc1C ; c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)cc1]

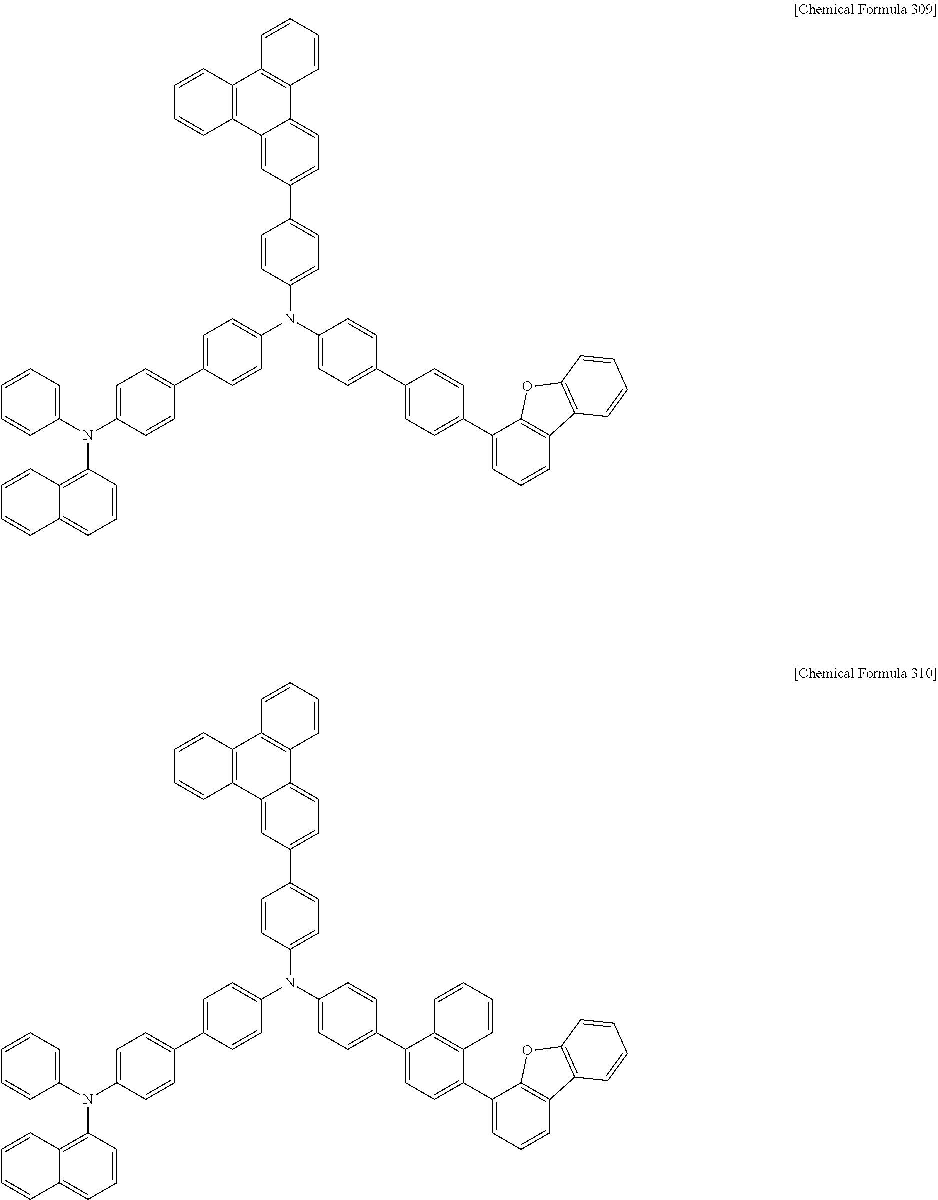 [SMILES: c1ccc(N(c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1]

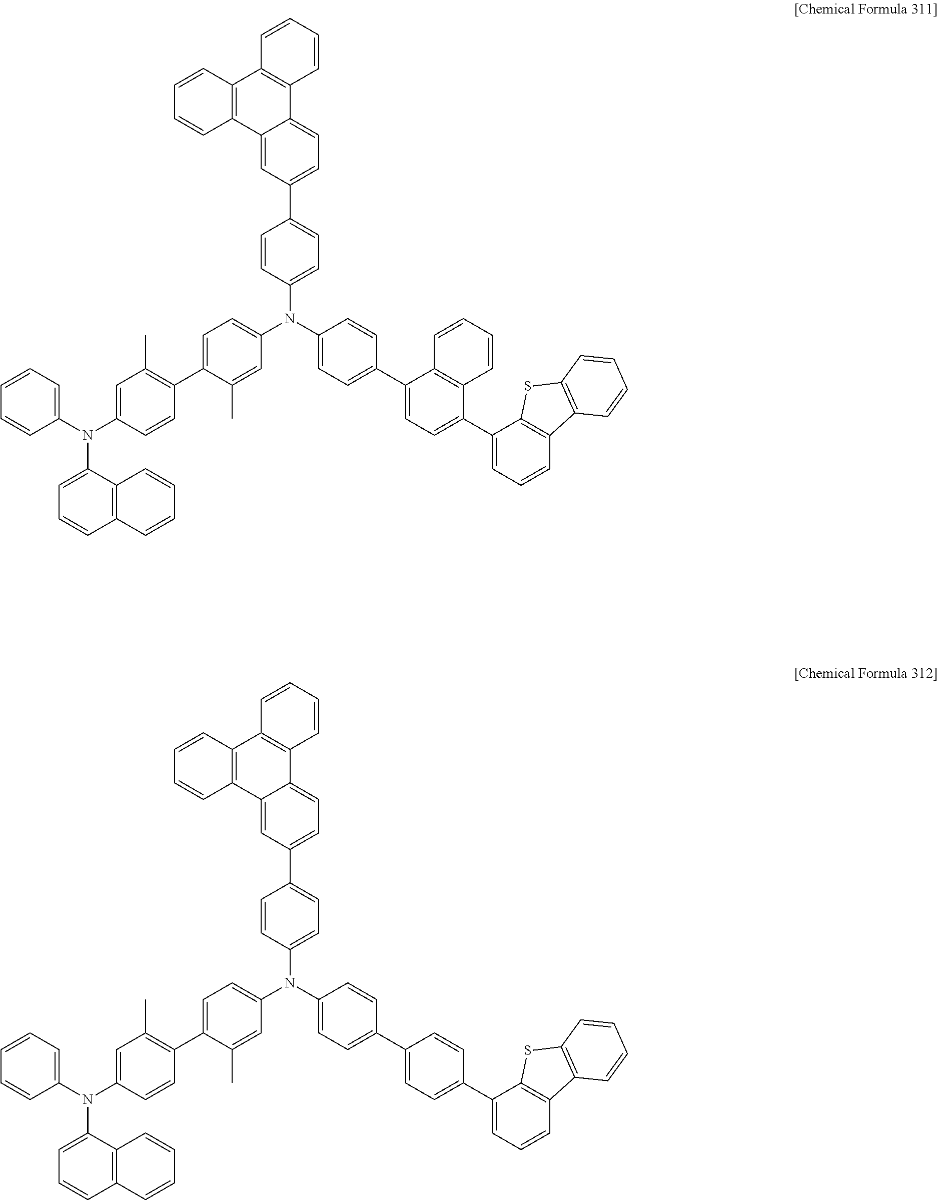 [SMILES: Cc1cc(N(c2ccc(-c3ccc4c5ccccc5c5ccccc5c4c3)cc2)c2ccc(-c3ccc(-c4cccc5c4sc4ccccc45)c4ccccc34)cc2)ccc1-c1ccc(N(c2ccccc2)c2cccc3ccccc23)cc1C ; Cc1cc(N(c2ccc(-c3ccc(-c4cccc5c4sc4ccccc45)cc3)cc2)c2ccc(-c3ccc4c5ccccc5c5ccccc5c4c3)cc2)ccc1-c1ccc(N(c2ccccc2)c2cccc3ccccc23)cc1C]

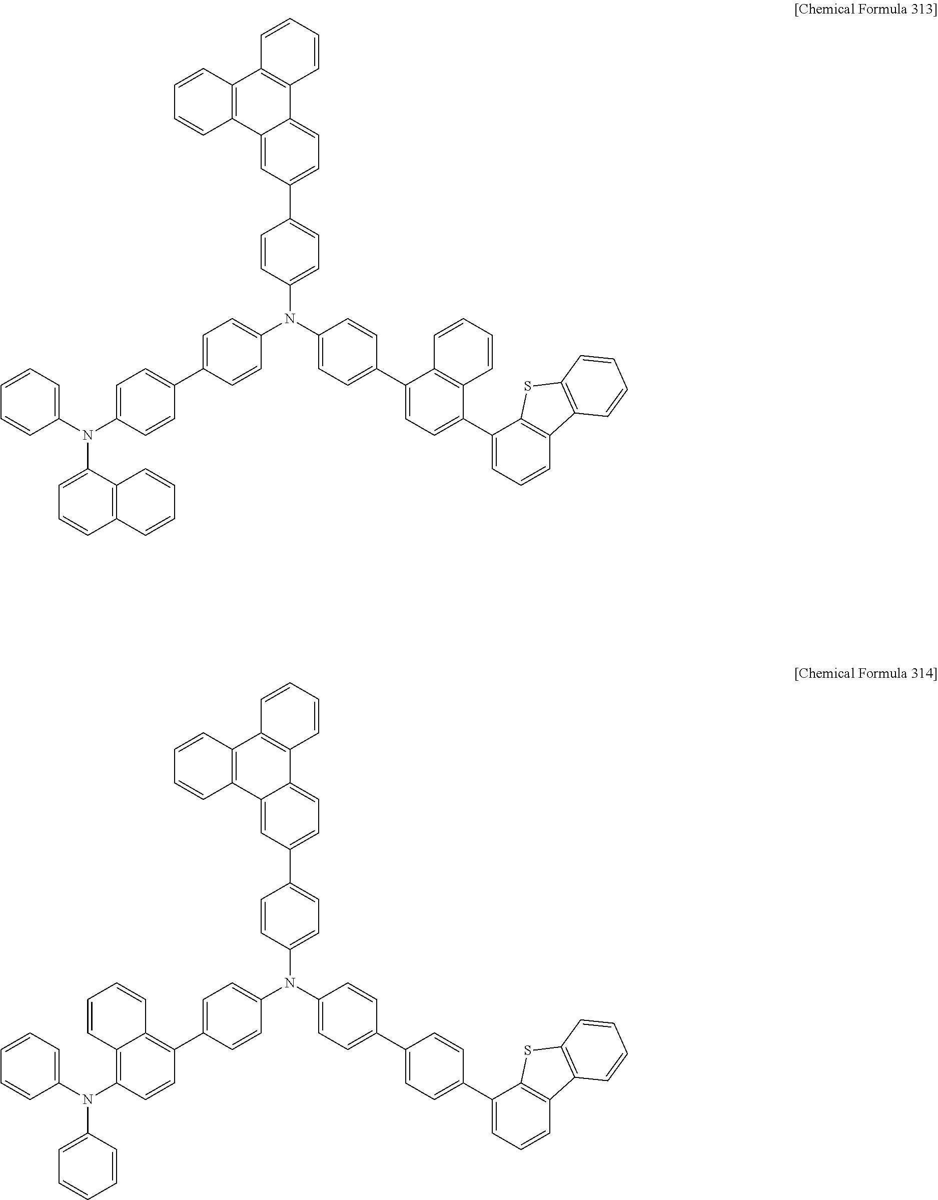 [SMILES: c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1 ; c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)c3ccccc23)cc1]

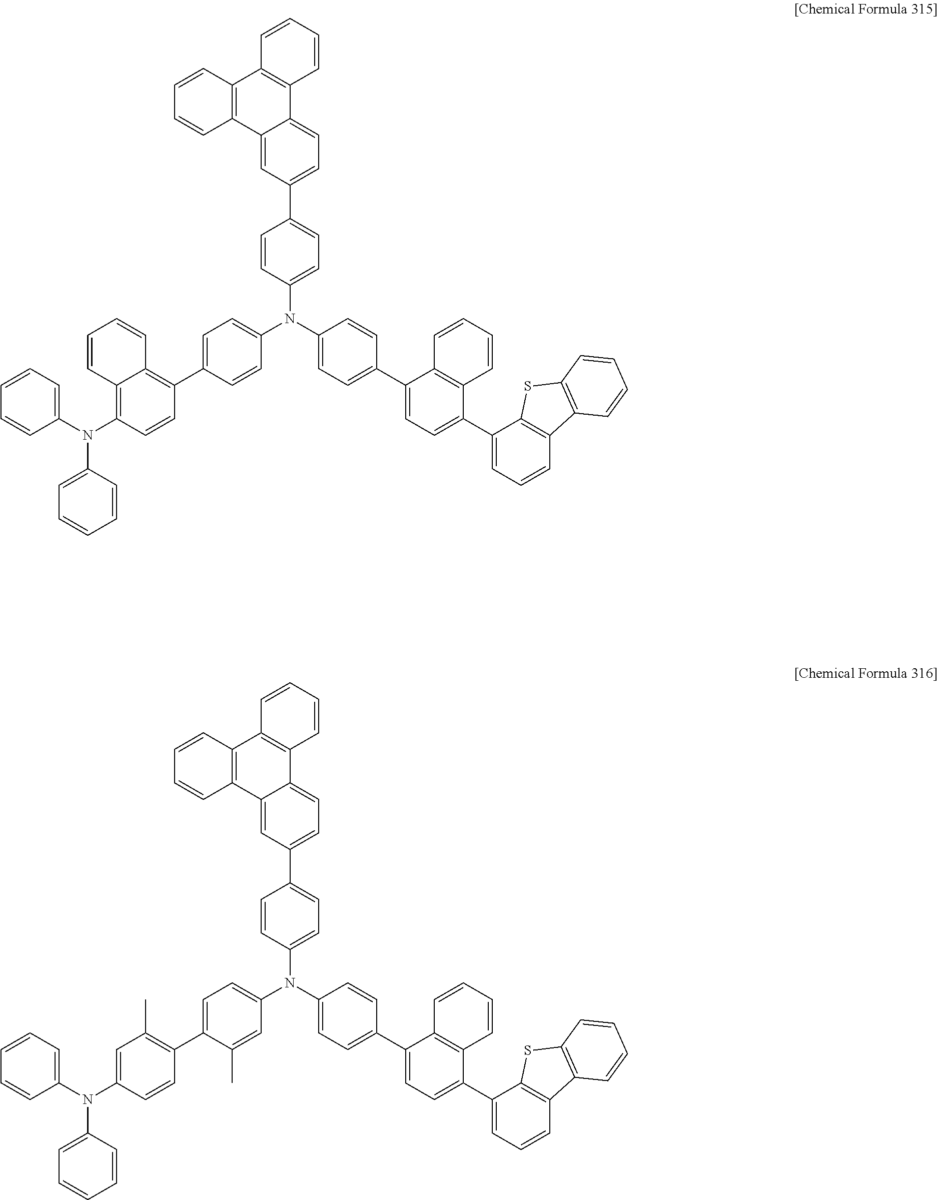 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4sc4ccccc45)c4ccccc34)cc2)cc1C]

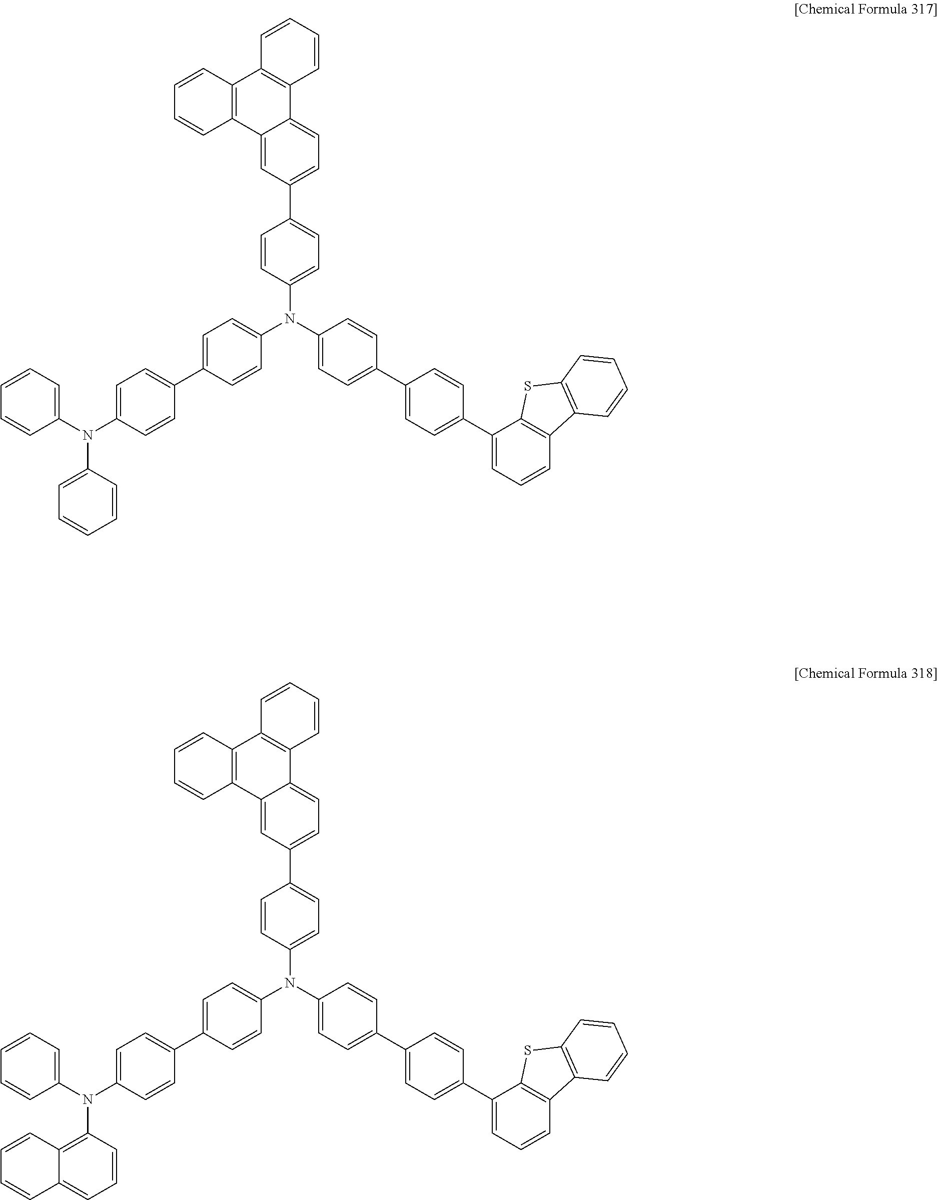 [SMILES: c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)c2cccc3ccccc23)cc1]

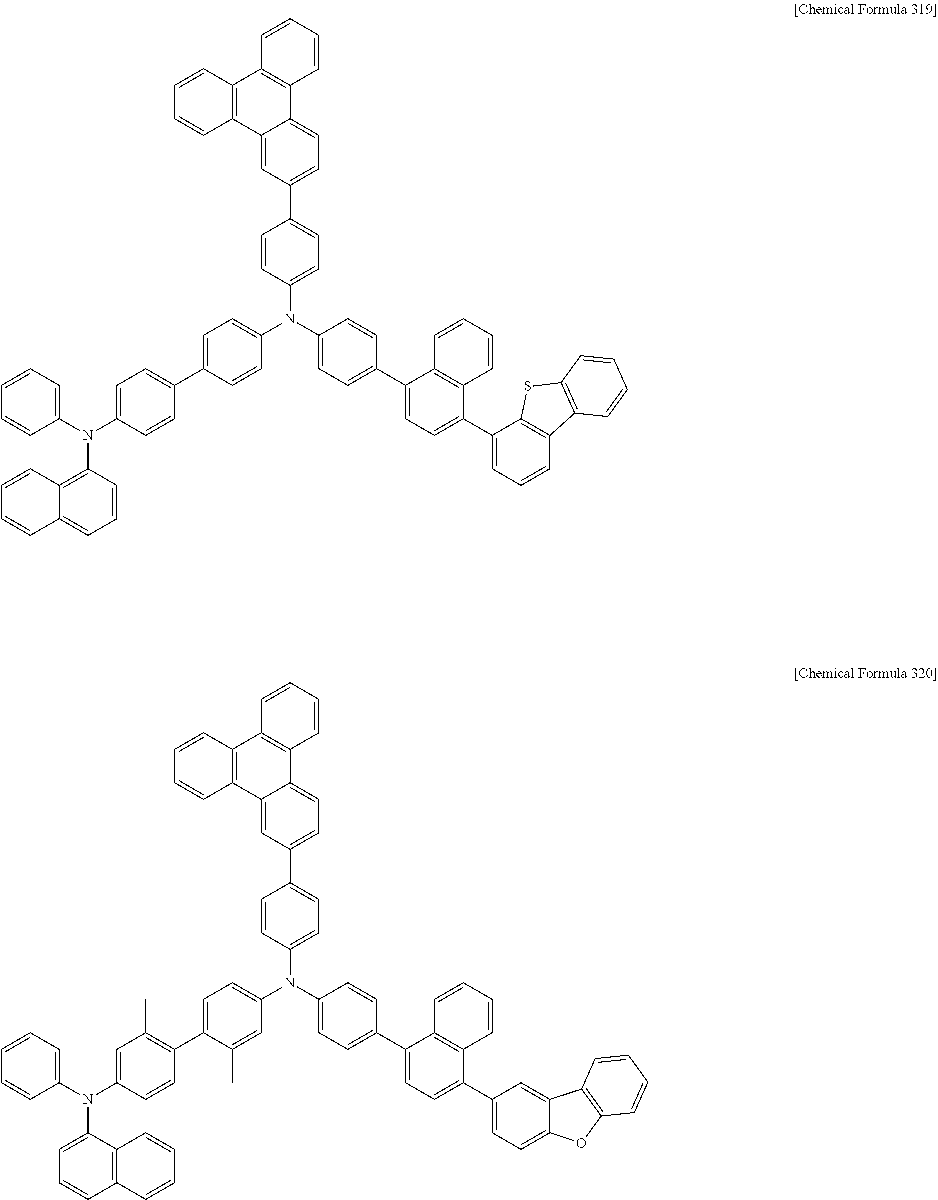 [SMILES: c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1 ; Cc1cc(N(c2ccc(-c3ccc4c5ccccc5c5ccccc5c4c3)cc2)c2ccc(-c3ccc(-c4ccc5oc6ccccc6c5c4)c4ccccc34)cc2)ccc1-c1ccc(N(c2ccccc2)c2cccc3ccccc23)cc1C]

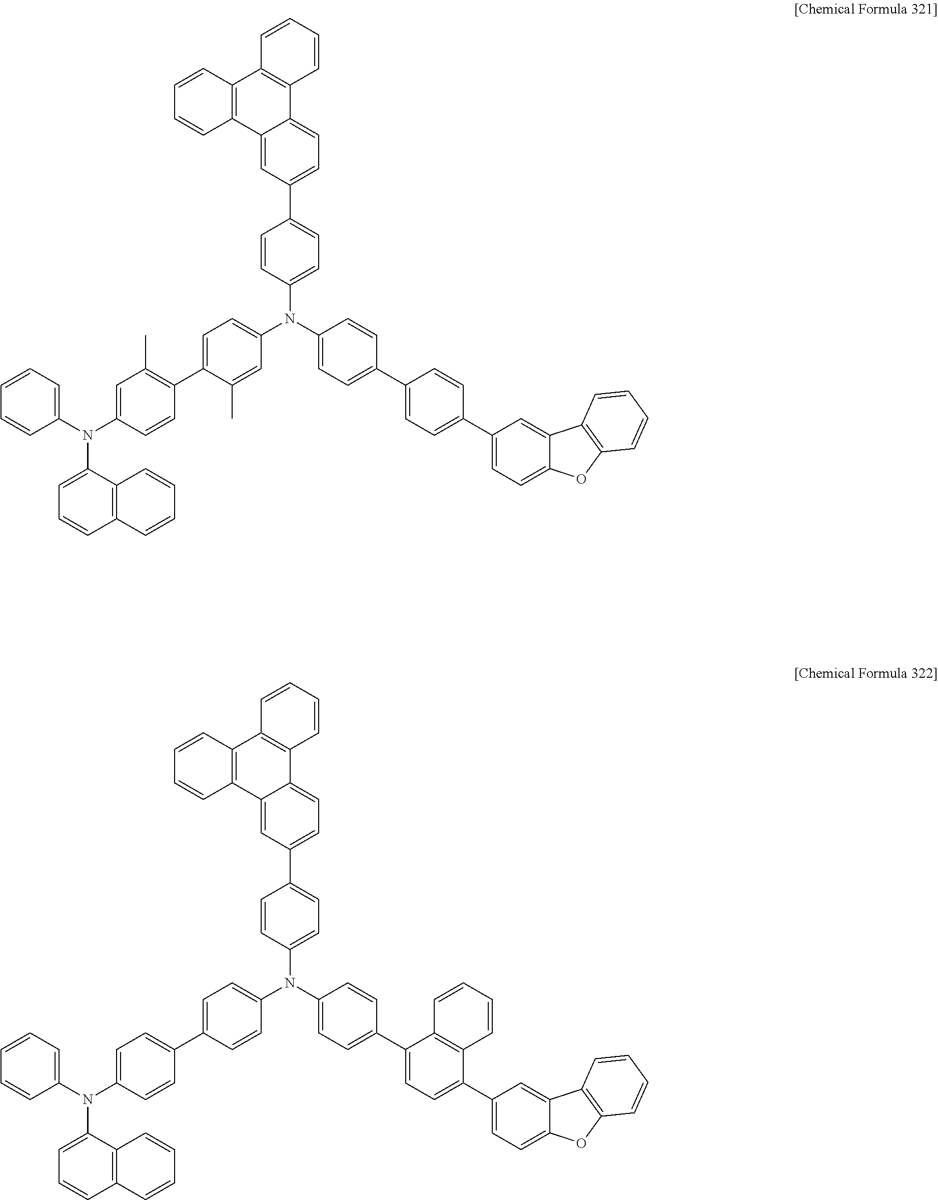 [SMILES: Cc1cc(N(c2ccc(-c3ccc(-c4ccc5oc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1]

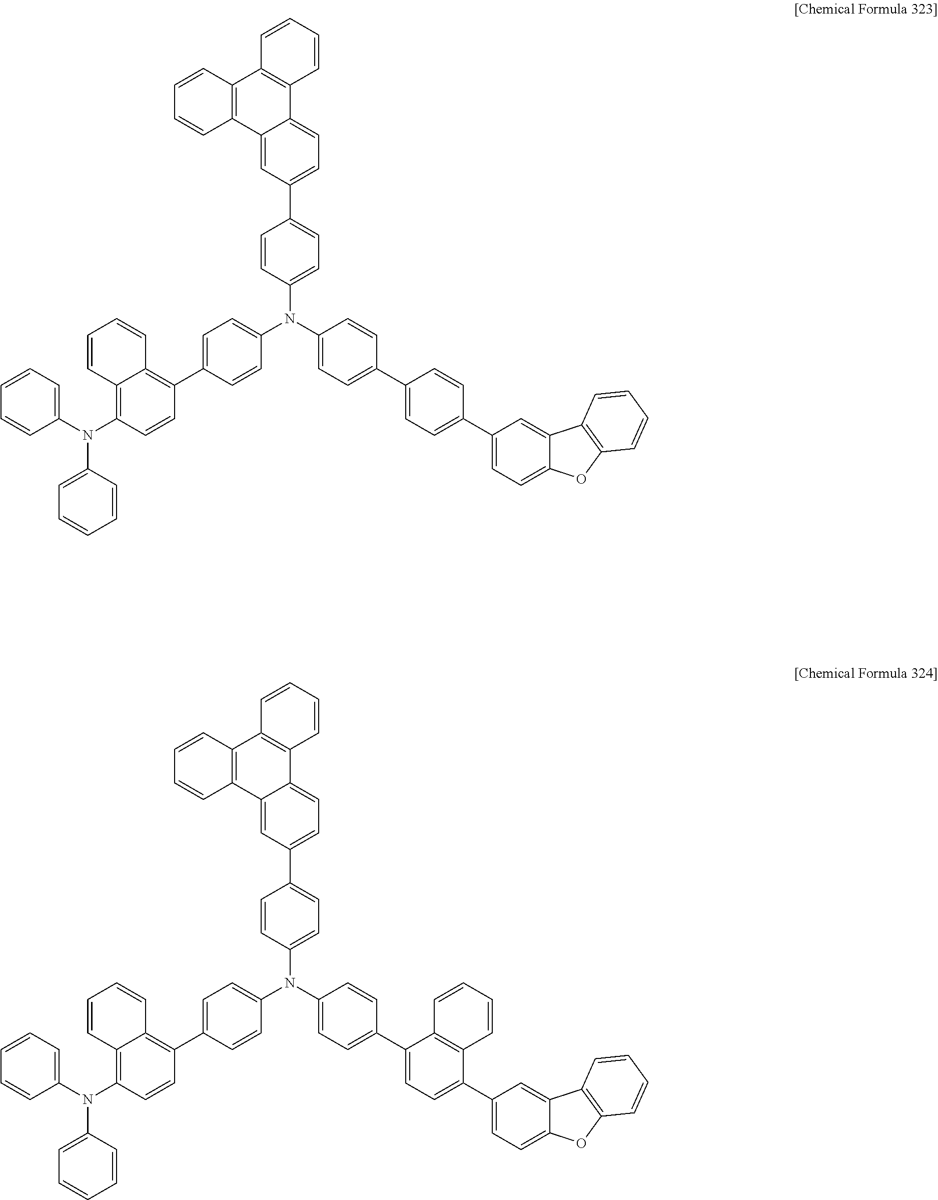 [SMILES: c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)c3ccccc23)cc1]

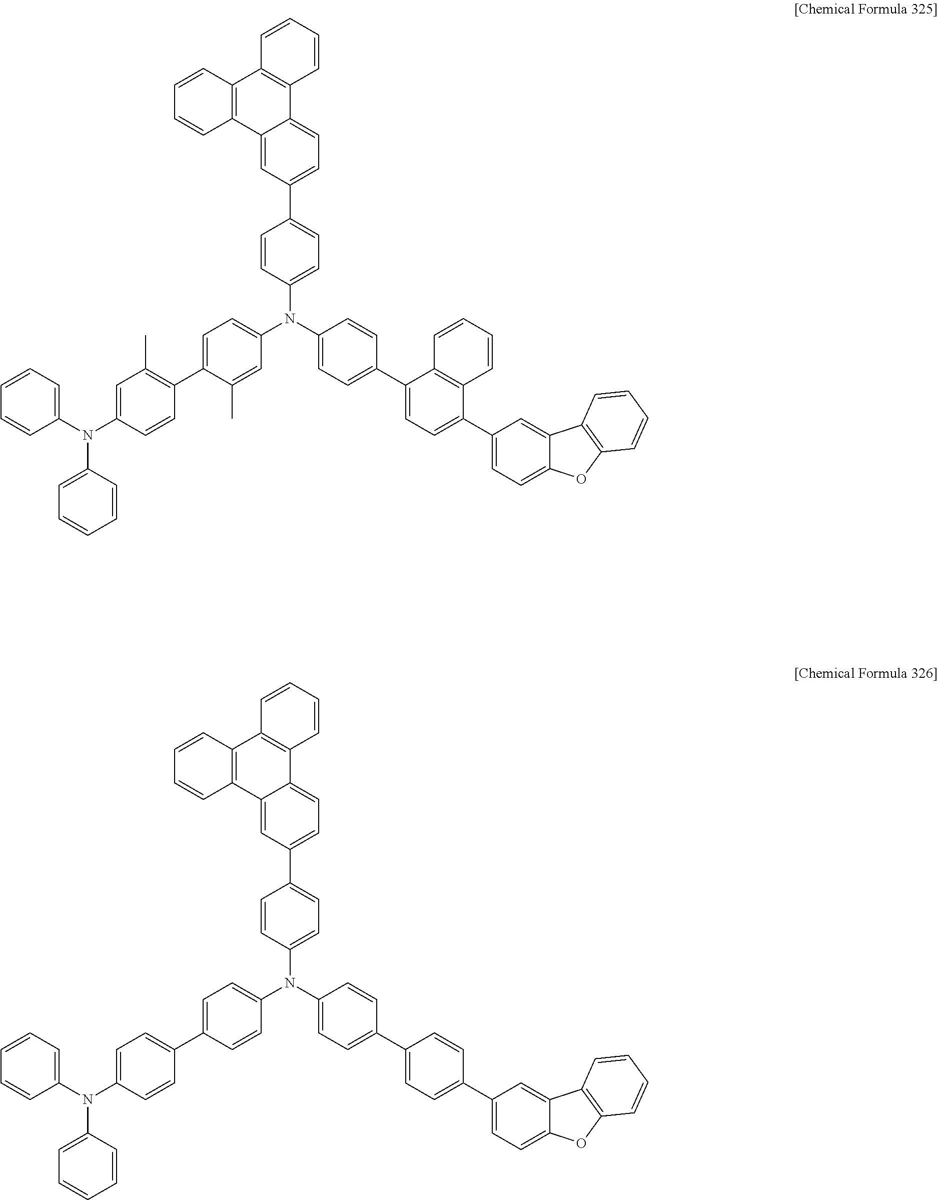 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5oc6ccccc6c5c4)c4ccccc34)cc2)cc1C ; c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)cc1]

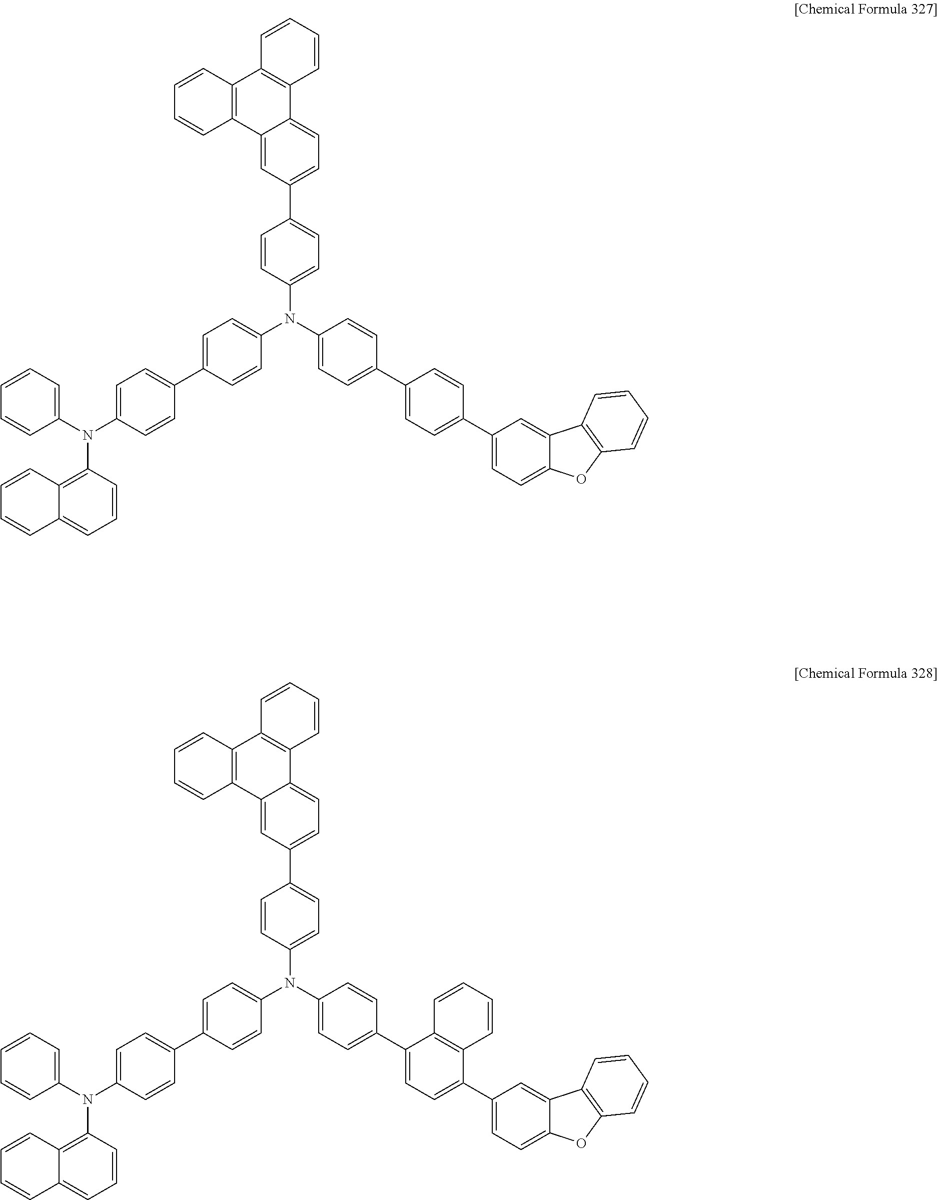 [SMILES: c1ccc(N(c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1]

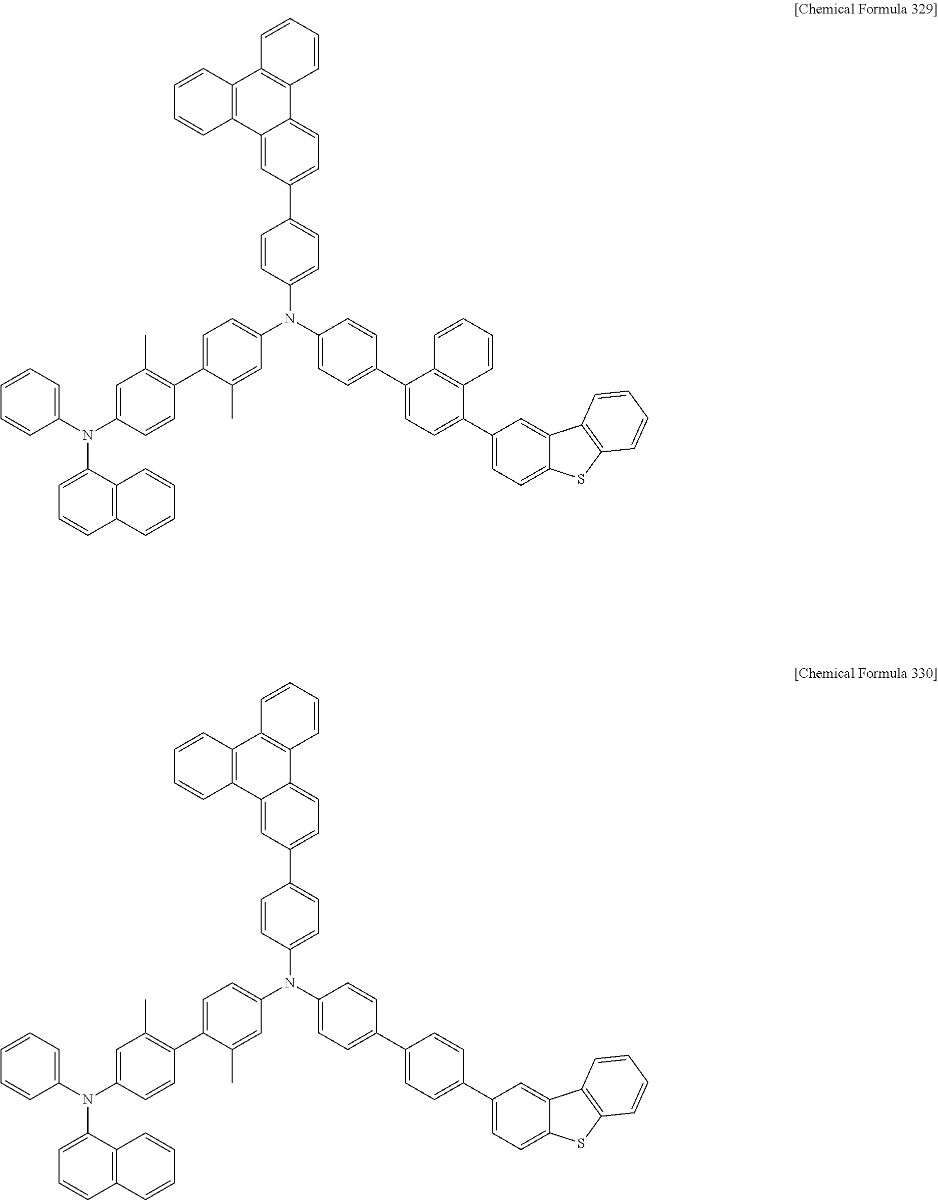 [SMILES: Cc1cc(N(c2ccc(-c3ccc4c5ccccc5c5ccccc5c4c3)cc2)c2ccc(-c3ccc(-c4ccc5sc6ccccc6c5c4)c4ccccc34)cc2)ccc1-c1ccc(N(c2ccccc2)c2cccc3ccccc23)cc1C ; Cc1cc(N(c2ccc(-c3ccc(-c4ccc5sc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C]

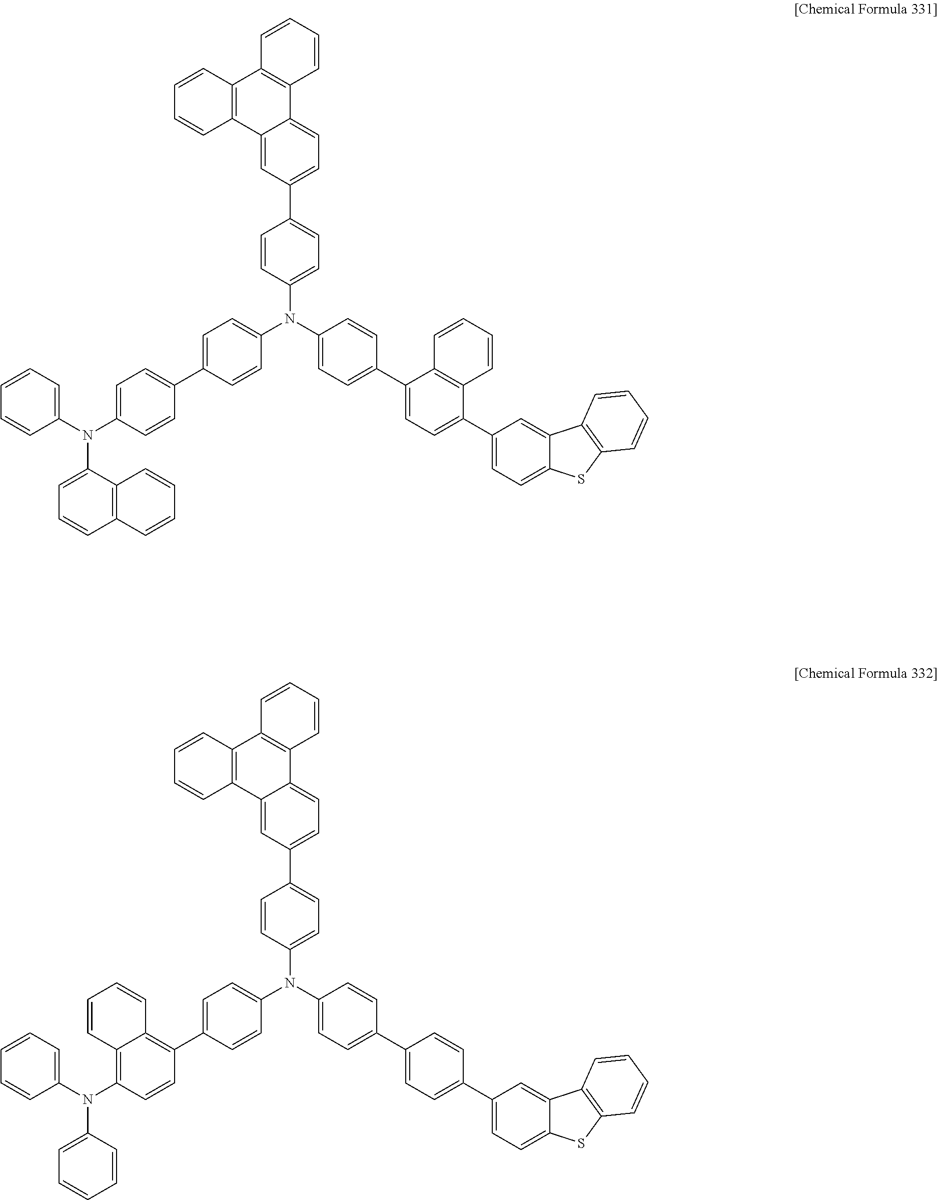 [SMILES: c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1 ; c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)c3ccccc23)cc1]

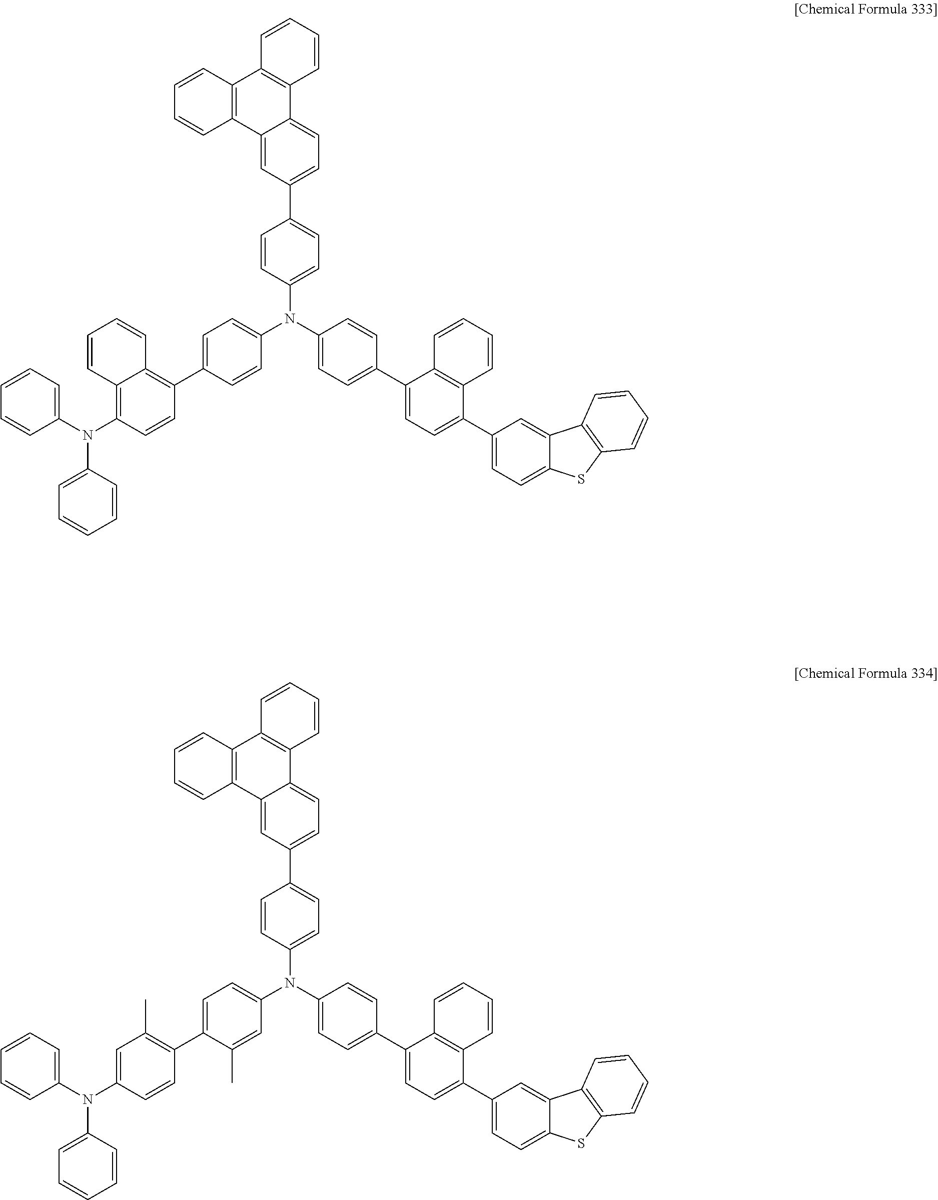 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5sc6ccccc6c5c4)c4ccccc34)cc2)cc1C]

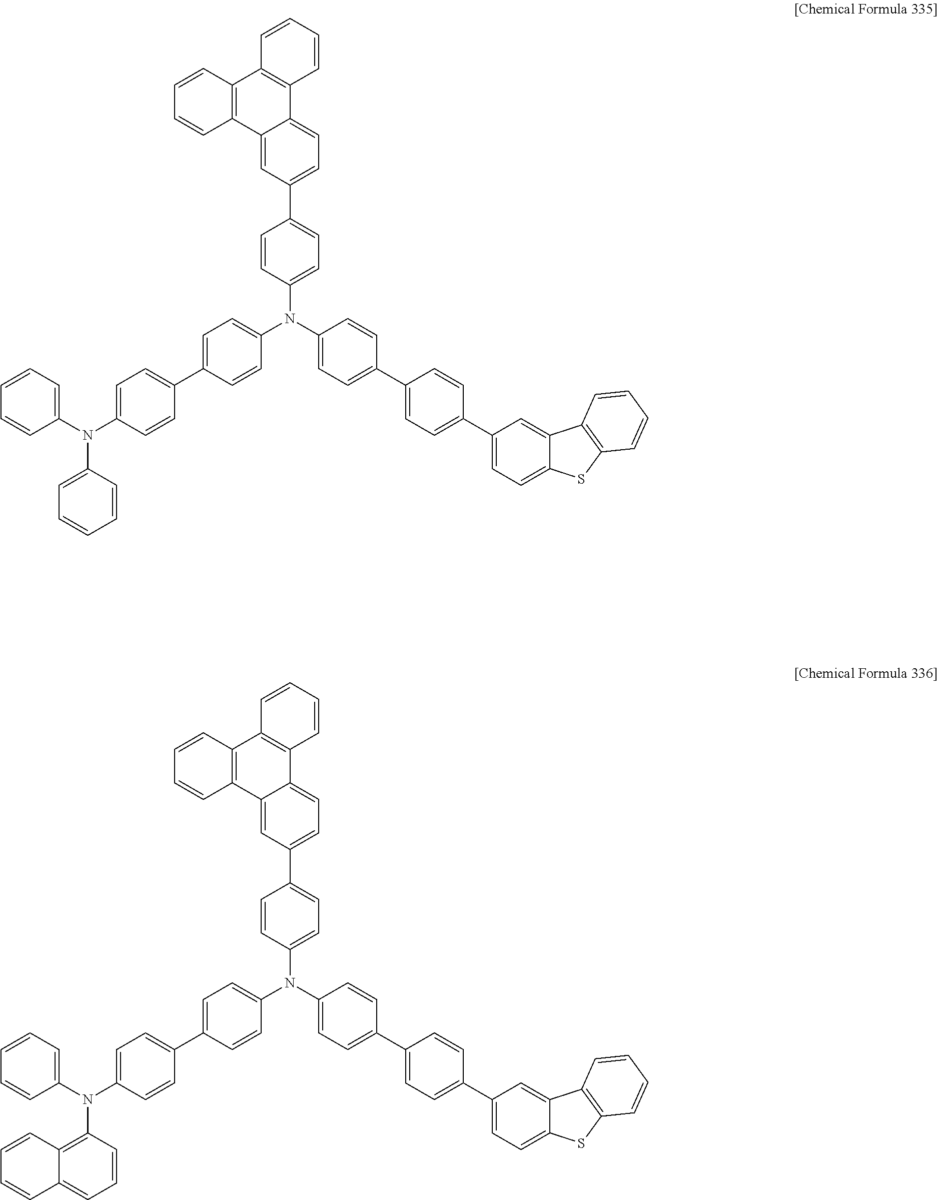 [SMILES: c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)c2cccc3ccccc23)cc1]

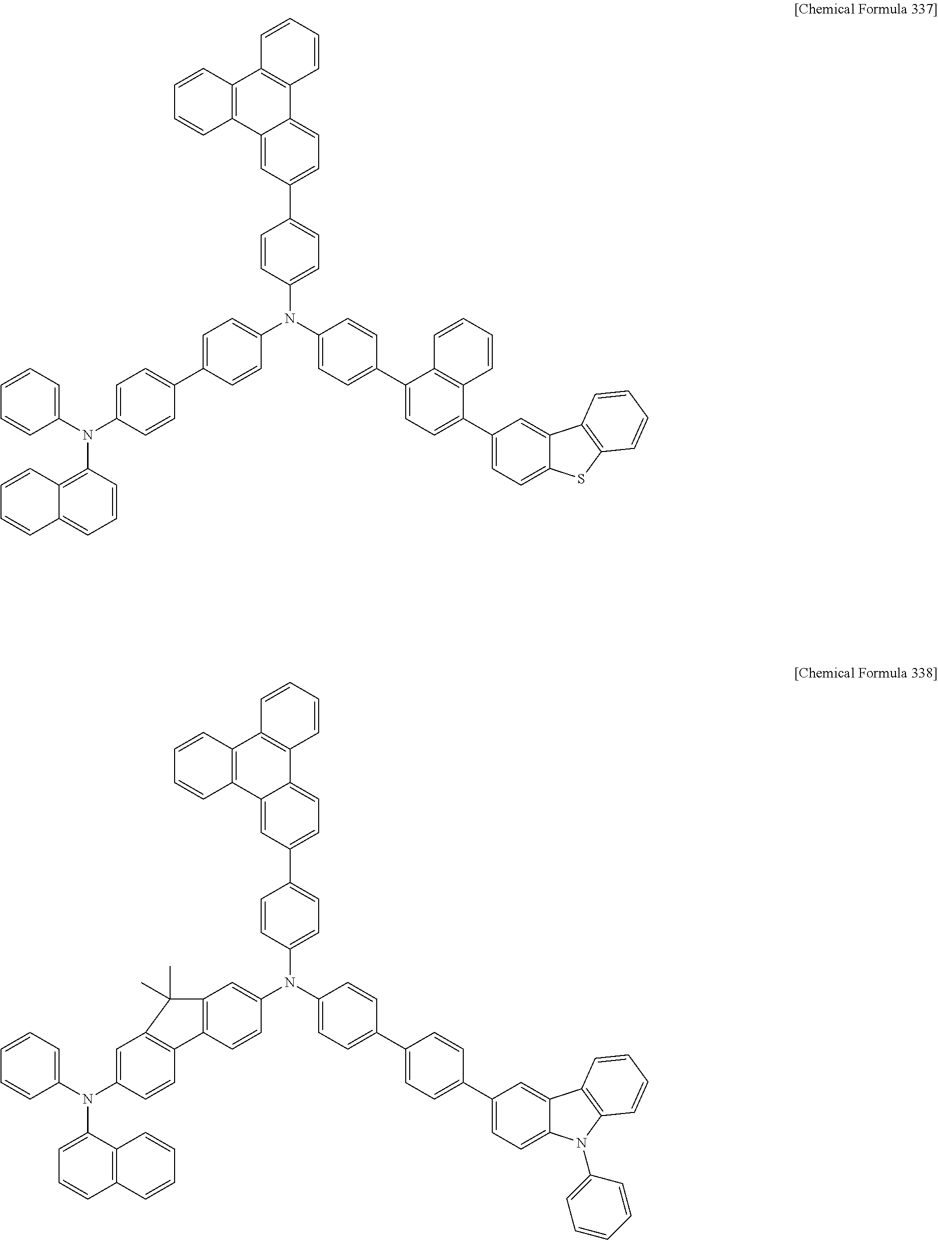 [SMILES: c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1 ; CC1(C)c2cc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)ccc2-c2ccc(N(c3ccccc3)c3cccc4ccccc34)cc21]

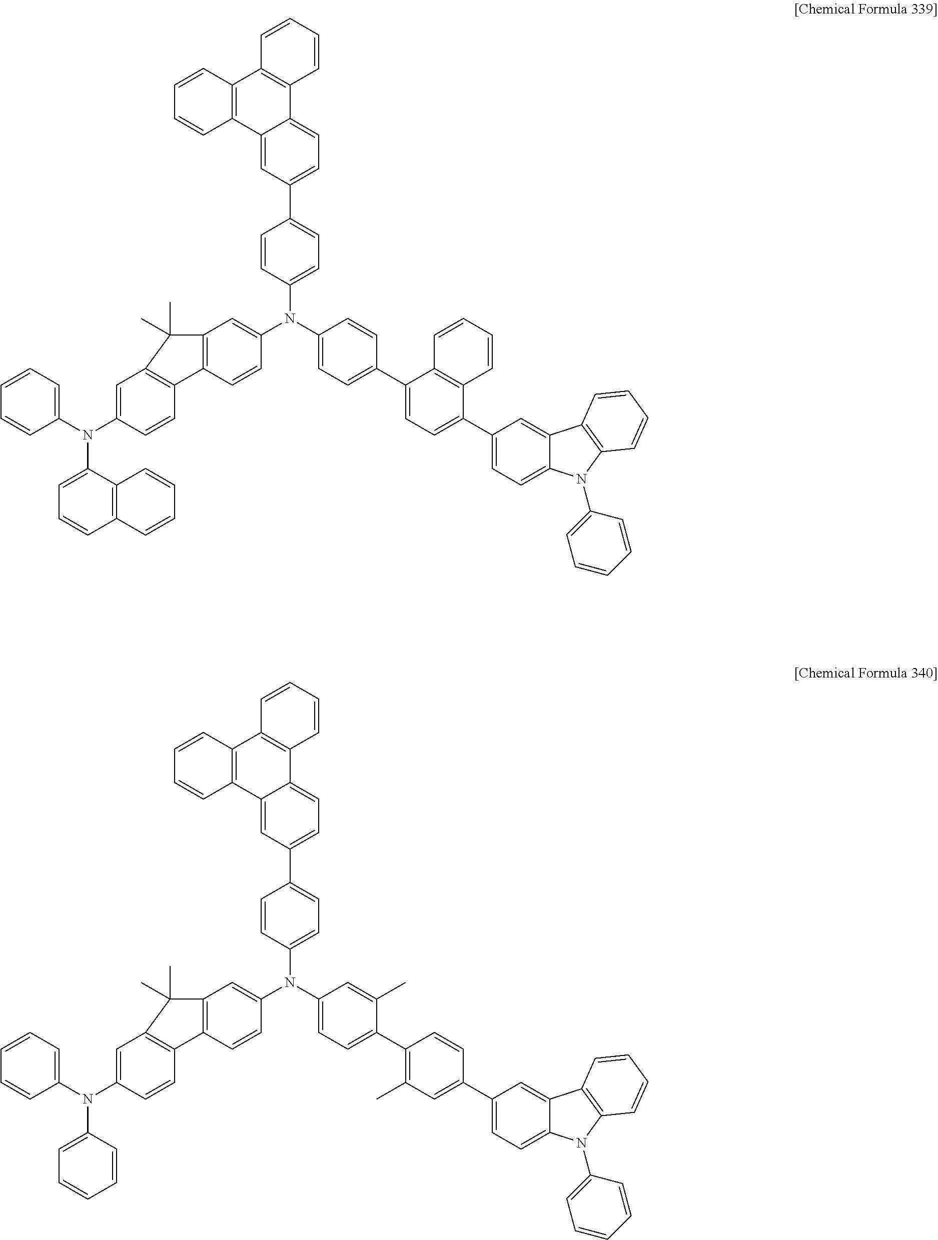 [SMILES: CC1(C)c2cc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)ccc2-c2ccc(N(c3ccccc3)c3cccc4ccccc34)cc21 ; Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

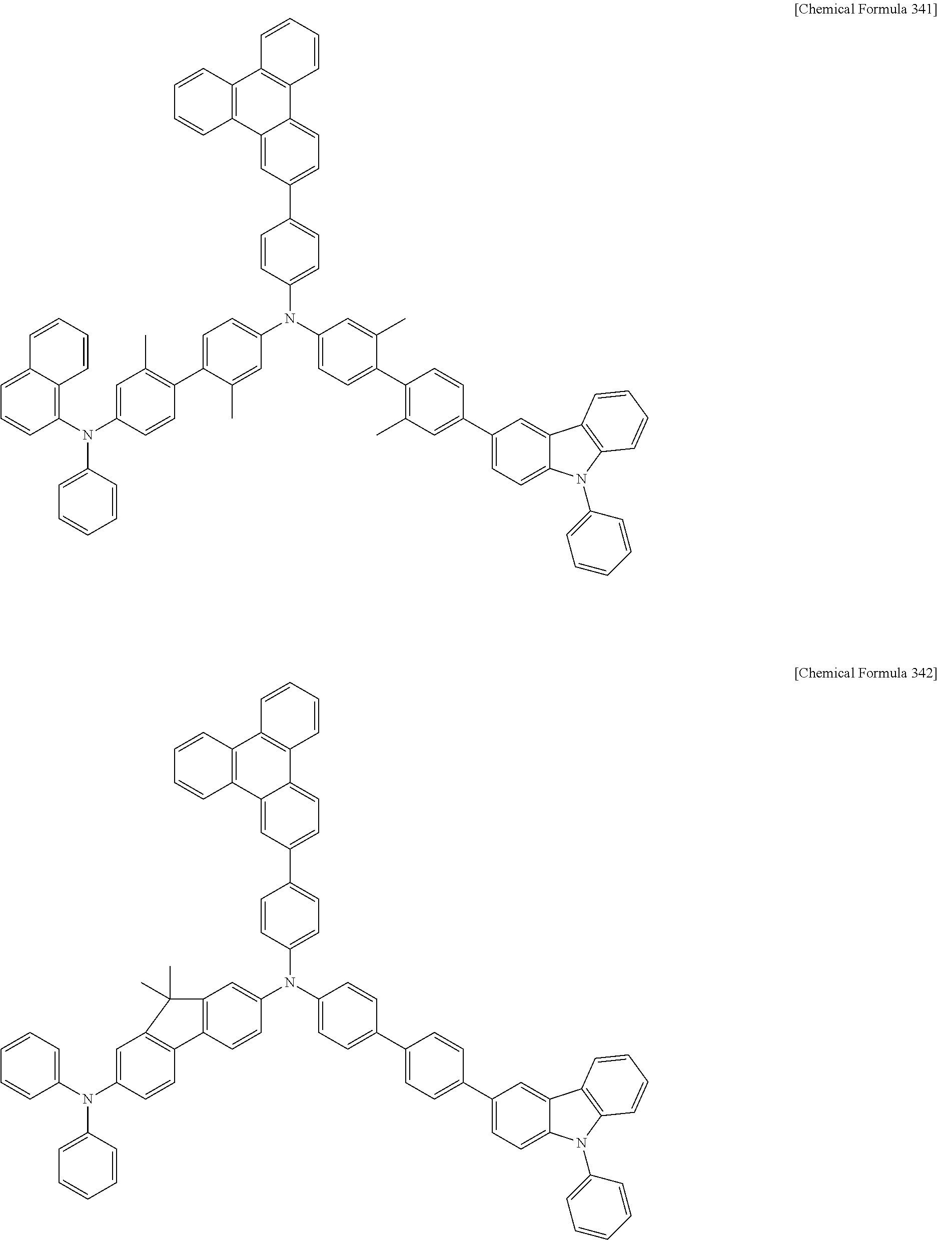 [SMILES: Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc(-c3ccc(N(c4ccccc4)c4cccc5ccccc45)cc3C)c(C)c2)cc1C ; CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)cc21]

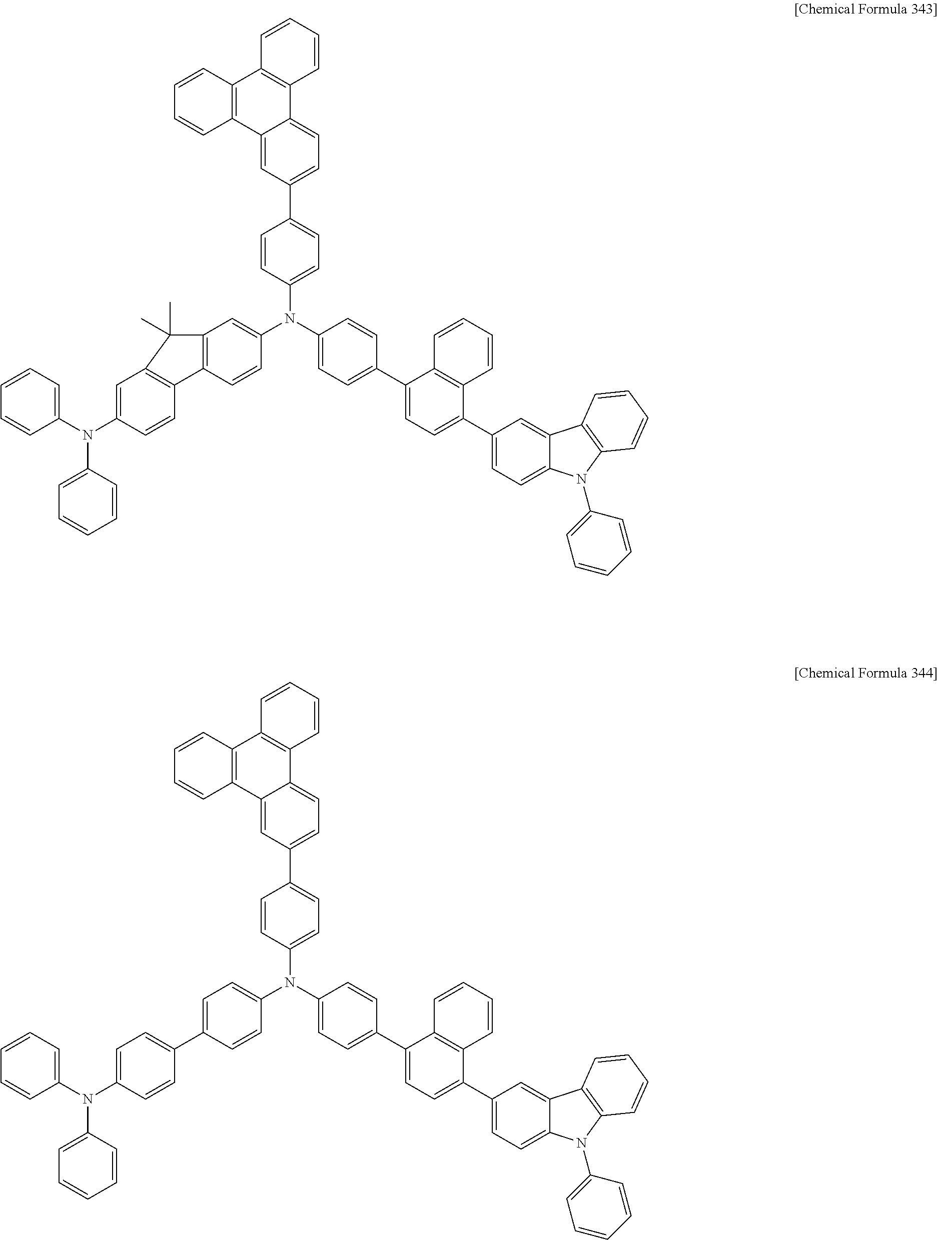 [SMILES: CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)cc21 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)cc2)cc1]

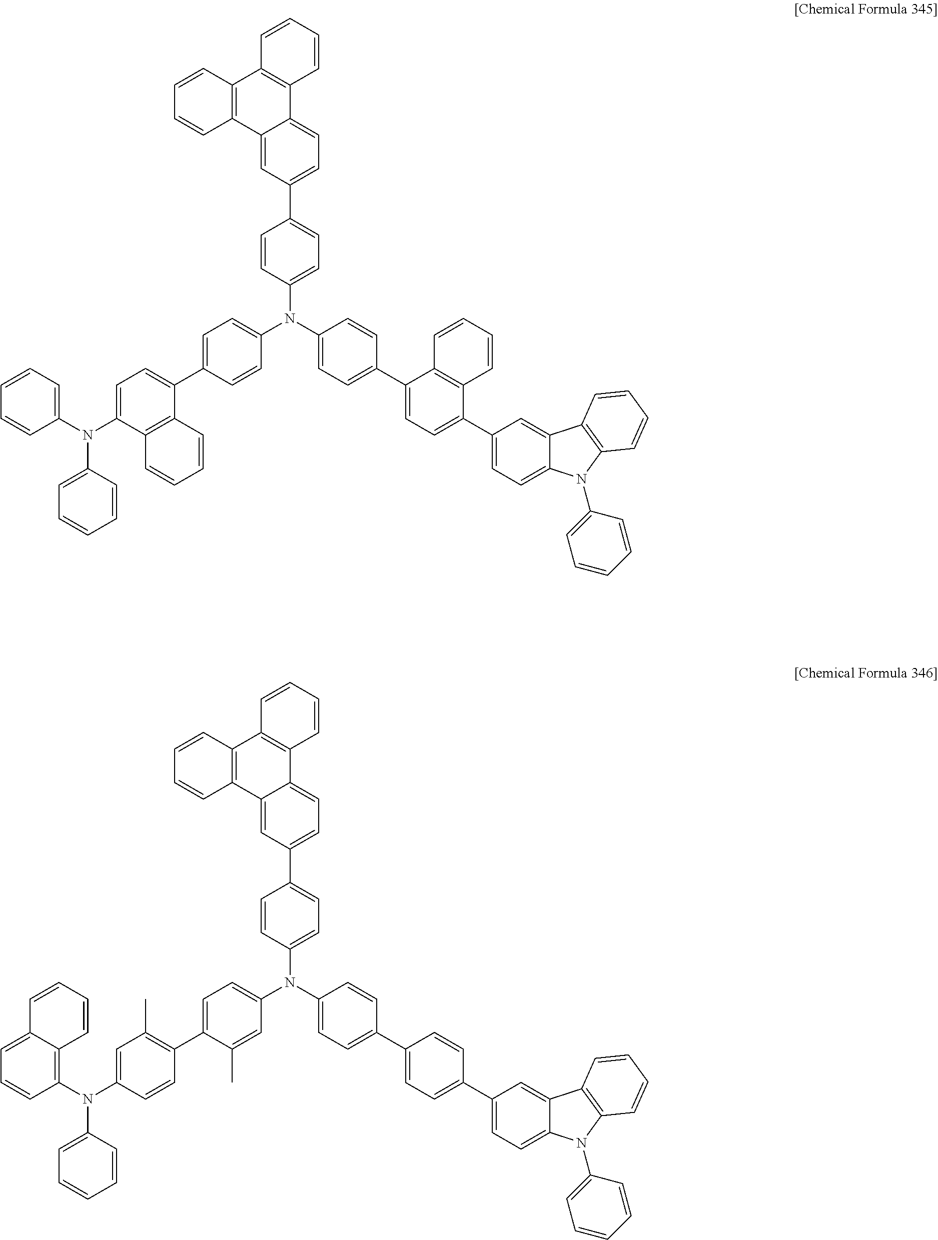 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)c3ccccc23)cc1 ; Cc1cc(N(c2ccc(-c3ccc(-c4ccc5c(c4)c4ccccc4n5-c4ccccc4)cc3)cc2)c2ccc(-c3ccc4c5ccccc5c5ccccc5c4c3)cc2)ccc1-c1ccc(N(c2ccccc2)c2cccc3ccccc23)cc1C]

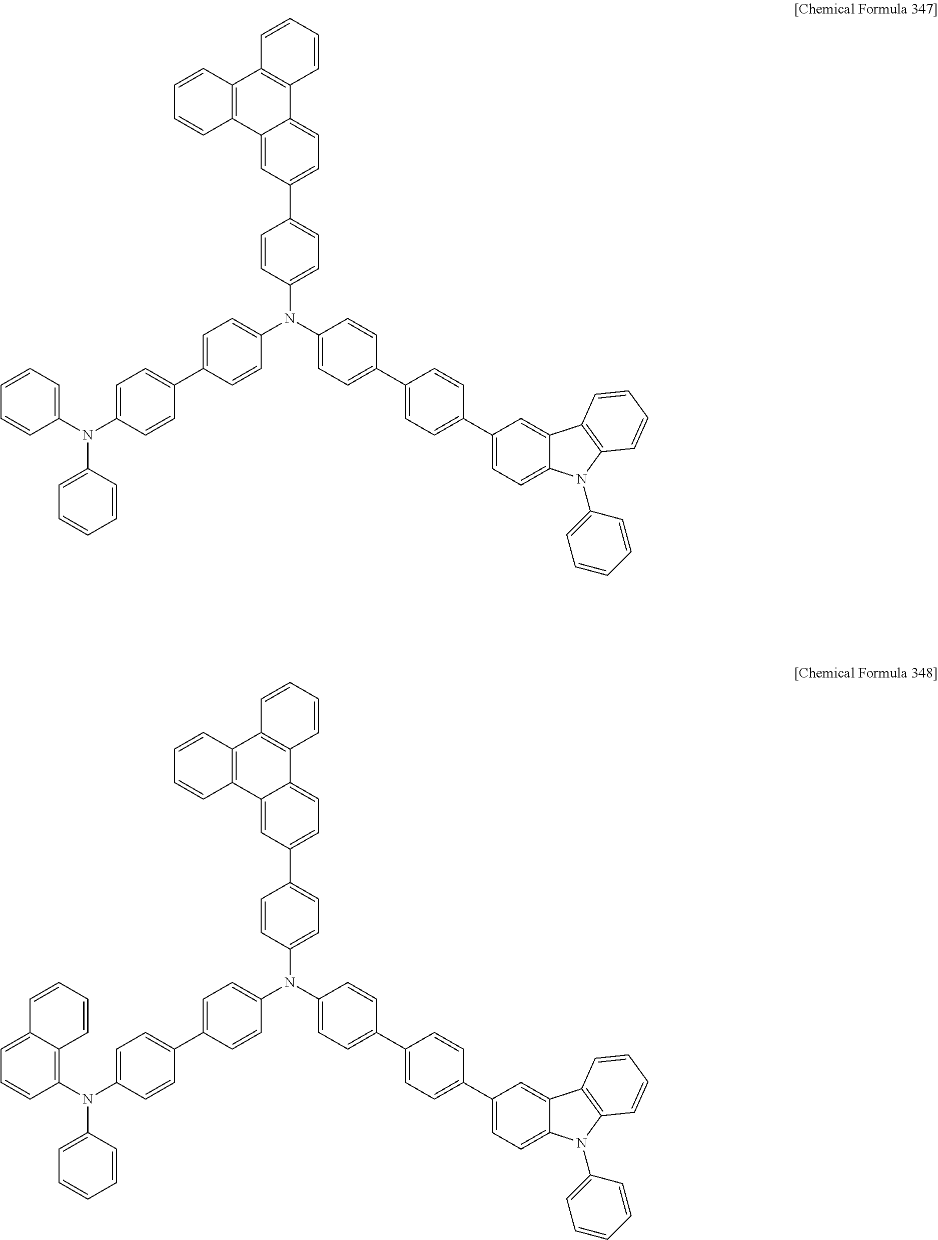 [SMILES: c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)c2cccc3ccccc23)cc1]

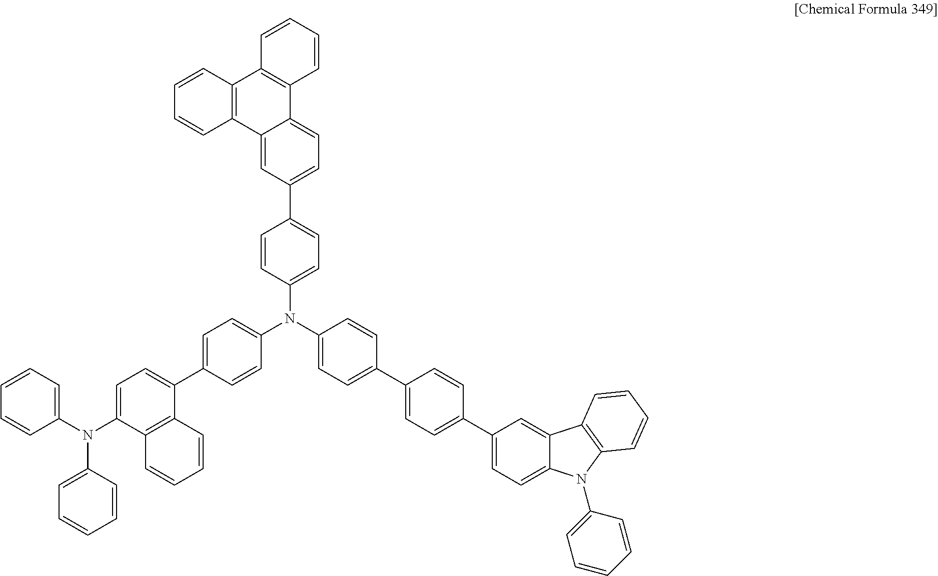 [SMILES: c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)c3ccccc23)cc1]

The compound for an organic photoelectric device including the above compounds may have a glass transition temperature of greater than or equal to about 110° C. and a thermal decomposition temperature of greater than or equal to about 400° C., indicating enhanced thermal stability. Thereby, it may be possible to produce an organic photoelectric device having a high efficiency.

The compound for an organic photoelectric device including the above compounds may play a role for emitting light or injecting and/or transporting electrons, and also act as a light emitting host with a dopant. Thus, the compound for an organic photoelectric device may be used as a phosphorescent or fluorescent host material, a blue light emitting dopant material, or an electron transport material. The compound for an organic photoelectric device according to an embodiment may be used for an organic thin layer. Thus, it may enhance the life-span characteristic, efficiency characteristic, electrochemical stability, and thermal stability of an organic photoelectric device and decrease the driving voltage.

According to another embodiment, an organic photoelectric device that includes the compound for an organic photoelectric device is provided. The organic photoelectric device may include an organic light emitting diode, an organic solar cell, an organic transistor, an organic photo conductor drum, an organic memory device, or the like. For example, the compound for an organic photoelectric device according to an embodiment may be included in an electrode or an electrode buffer layer in an organic solar cell to enhance quantum efficiency, and it may be used as an electrode material for a gate, a source-drain electrode, or the like in an organic transistor.

According to another embodiment, an organic light emitting diode includes an anode, a cathode, and an organic thin layer between the anode and the cathode. The organic thin layer may include the compound for an organic photoelectric device according to an embodiment. The organic thin layer may include one or more of an emission layer, a hole transport layer (HTL), a hole injection layer (HIL), an electron transport layer (ETL), an electron injection layer (EIL), or a hole blocking layer. In an implementation, the compound for an organic photoelectric device according to an embodiment may be included in an electron transport layer (ETL) or an electron injection layer (EIL). In an implementation, the compound for an organic photoelectric device according to an embodiment may be included in an emission layer, e.g., as a phosphorescent or fluorescent host, or as a fluorescent blue dopant material.

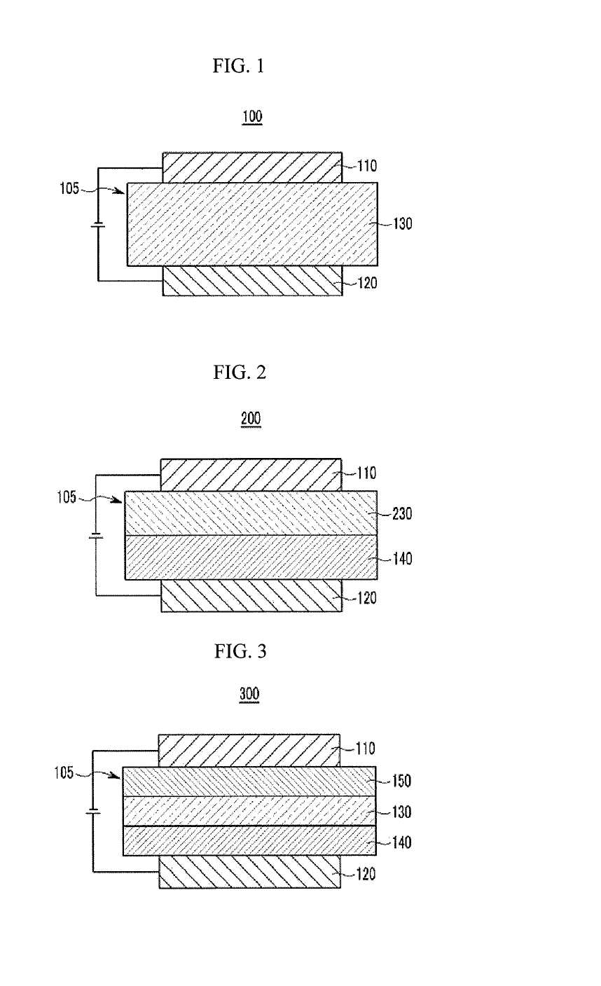

FIGS. 1 to 5 illustrate cross-sectional views showing organic light emitting diodes including a compound for an organic photoelectric device according to an embodiment. Referring to FIGS. 1 to 5, organic light emitting diodes 100, 200, 300, 400, and 500 according to an embodiment include an organic thin layer 105 interposed between an anode 120 and a cathode 110.

The anode 120 may include an anode material laving a large work function to help hole injection into an organic thin layer. The anode material may include, e.g.: a metal such as nickel, platinum, vanadium, chromium, copper, zinc, and gold, or alloys thereof; a metal oxide such as zinc oxide, indium oxide, indium tin oxide (ITO), or indium zinc oxide (IZO); a combined metal and oxide such as ZnO:Al or SnO₂:Sb; or a conductive polymer such as poly(3-methylthiophene), poly[3,4-(ethylene-1,2-dioxy)thiophene] (PEDT), polypyrrole, or polyaniline; etc. A transparent electrode including indium tin oxide (ITO) may be included as an anode.

The cathode 110 may include a cathode material having a small work function to help electron injection into an organic thin layer. The cathode material may include, e.g.: a metal such as magnesium, calcium, sodium, potassium, titanium, indium, yttrium, lithium, gadolinium, aluminum, silver, tin, or lead, or alloys thereof; or a multi-layered material such as LiF/Al, Liq/Al, LiO₂/Al, LiF/Ca, LiF/Al, or BaF₂/Ca; etc. A metal electrode including aluminum may be included as a cathode.

In the example embodiment shown in FIG. 1, the organic light emitting diode 100 includes an organic thin layer 105 including only an emission layer 130.

In the example embodiment shown in FIG. 2, a double-layered organic light emitting diode 200 includes an organic thin layer 105 including an emission layer 230 including an electron transport layer (ETL), and a hole transport layer (HTL) 140. As shown in FIG. 2, the organic thin layer 105 includes a double layer of the emission layer 230 and hole transport layer (HTL) 140. The emission layer 130 also functions as an electron transport layer (ETL), and the hole transport layer (HTL) 140 layer has an excellent binding property with a transparent electrode such as ITO or an excellent hole transport capability.

In the example embodiment shown in FIG. 3, a three-layered organic light emitting diode 300 includes an organic thin layer 105 including an electron transport layer (ETL) 150, an emission layer 130, and a hole transport layer (HTL) 140. The emission layer 130 is independently installed, and layers having an excellent electron transport capability or an excellent hole transport capability are separately stacked.

In the example embodiment shown in FIG. 4, a four-layered organic light emitting diode 400 includes an organic thin layer 105 including an electron injection layer (EIL) 160, an emission layer 130, a hole transport layer (HTL) 140, and a hole injection layer (HIL) 170 for adherence with the anode of ITO.

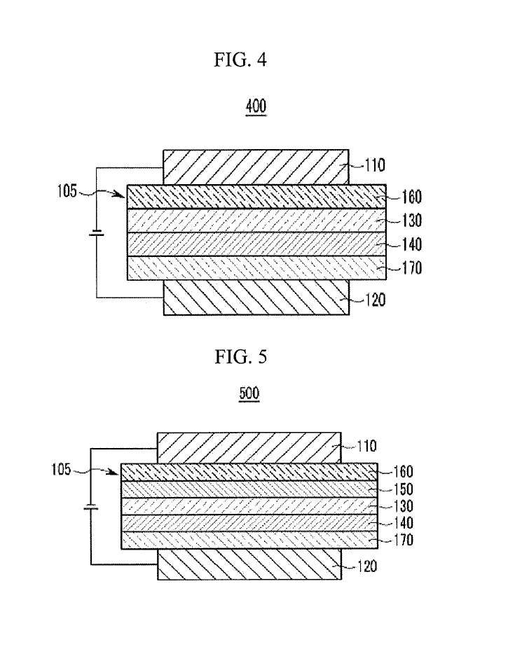

In the example embodiment shown in FIG. 5, a five layered organic light emitting diode 500 includes an organic thin layer 105 including an electron transport layer (ETL) 150, an emission layer 130, a hole transport layer (HTL) 140, and a hole injection layer (HIL) 170, and further includes an electron injection layer (EIL) 160 to achieve a low voltage.

In the example embodiments shown in FIGS. 1 to 5, the organic thin layer 105 including at least one selected from the group of an electron transport layer (ETL) 150, an electron injection layer (EIL) 160, emission layers 130 and 230, a hole transport layer (HTL) 140, a hole injection layer (HIL) 170, and combinations thereof includes a compound for an organic photoelectric device. The compound for an organic photoelectric device may be used for an electron transport layer (ETL) 150 including the electron transport layer (ETL) 150 or electron injection layer (EIL) 160. When it is used for the electron transport layer (ETL), it may be possible to provide an organic light emitting diode having a more simple structure by avoiding the use of an additional hole blocking layer.

When the compound for an organic photoelectric device is included in the emission layers 130 and 230, the material for the organic photoelectric device may be included as a phosphorescent or fluorescent host or a fluorescent blue dopant.

The organic light emitting diode may be fabricated by, e.g.,: forming an anode on a substrate; forming an organic thin layer in accordance with a dry coating method such as evaporation, sputtering, plasma plating, and ion plating or a wet coating method such as spin coating, dipping, and flow coating; and providing a cathode thereon.

Another embodiment provides a display device including the organic light emitting diode according to an embodiment.

The following Examples and Comparative Examples are provided in order to highlight characteristics of one or more embodiments, but it will be understood that the Examples and

Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

(Preparation of Compound for Organic Photoelectric Device)

### Synthesis of Intermediates A, B, C, D, E, F, G, and H

Intermediates A, B, C, and D were synthesized according to the following Reaction Scheme 1.

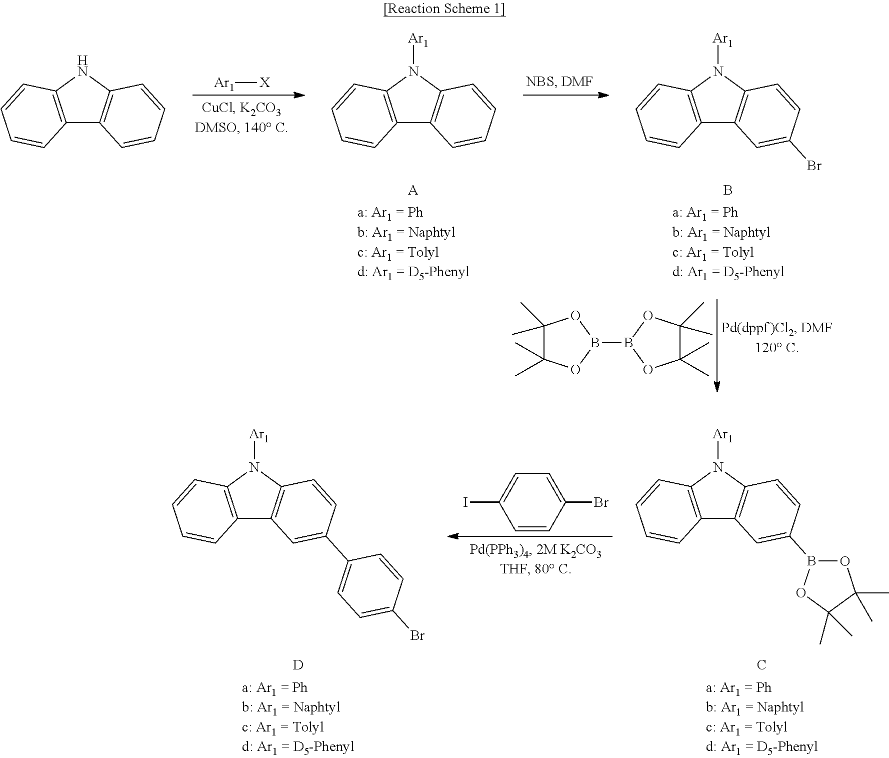

### Synthesis of Intermediates Aa and Ab

50 mmol of carbazole and 60 mmol of iodobenzene or 1-bromonaphthalene were dissolved in 500 mL of DMSO. The resultant solution was added to a mixed reaction solution prepared by dissolving 5.5 mmol of CuCl and 52 mmol of K₂CO₃. The mixture is agitated at 140° C. for 24 hours and then adsorption-filtered using Celite. The filtered solution was concentrated under a reduced pressure condition and then purified using a silica gel column chromatography. The purified product was recrystallized under a hexane or ether/methanol condition, respectively obtaining 55 g of Aa (phenyl, GC Mass (M+H⁺)=244.12) and 56 g of Ab (naphthyl, LC Mass (M+H+)=294.17).

### Synthesis of Intermediate Ac

24.3 g (yield of 79%) of a white solid intermediate Ac was prepared according to the same method as the method of preparing the intermediate Aa except for using 4-bromotoluene.

LCMass (a measured value: M+H+=294.17)

### Synthesis of Intermediate Ad

24.1 g (yield 81%) of a white solid intermediate Ac was prepared according to the same method as the method of preparing the intermediate Aa except for using 4-bromotoluene.

LCMass (a measured value: M+H+=249)

### Synthesis of Intermediate Ba

50 g of the intermediate Aa (N-phenylcarbazole) was dissolved in 400 mL of DMF, and the solution was added in a dropwise fashion to a solution prepared by dissolving 37.7 g of NBS (N-bromosuccinimide) in 100 mL of DMF. The mixture was reacted for 16 hours at room temperature and added to 1 L of MeOH. The resulting mixture was filtered to obtain a precipitate. Next, 500 mL of MeOH was added to the filtered solution, obtaining a precipitate.

The precipitate was recrystallized in hexane, obtaining 59 g (89%) of a desired product Ba (phenyl, GC Mass (M+H+)=322.06, 324.05).

### Synthesis of Intermediate Ca

55 g of the intermediate Ba and 65 g of bispinacolatodiborane were dissolved in 800 mL of DMF, and then 6.2 g of a Pd(dppf)Cl₂ catalyst and 25.1 g of potassium acetate (CH₃COOK) was added thereto. The resulting mixture was heated up to 120° C. in a reflux condenser under a nitrogen atmosphere and reacted for 18 hours. Then, a product remaining after removing DMF therefrom under a reduced pressure was dissolved in CH₂Cl₂. The solution was filtered using a filter filled with Celite, and the filtered solution was concentrated under a reduced pressure. The concentrated product was primarily purified using a silica gel column chromatography and recrystallized in hexane, obtaining product 40.2 g (64%) of Ca (Ca: phenyl, GC Mass (M+H+)=370.28).

### Synthesis of Intermediate Da

40 g of the intermediate Ca, 34 g of 4-iodo-1-bromobenzene, and 3.7 g of tetrakistriphenylphosphine palladium were dissolved in 600 mL of THF in a 2 L 3-necked round-bottomed flask, and 250 mL of K₂CO₃ in a concentration of 2 M was added thereto. Next, the resulting mixture was heated up to 80° C. in a reflux condenser under a nitrogen atmosphere and agitated for 15 hours. Then, a water layer therein was removed, and THF was removed therefrom. The remaining product was dissolved in CH₂Cl₂, and a charcoal powder was added thereto. Then, the mixture was agitated. The agitated mixture was filtered using a filter filled with Celite and then concentrated under reduced pressure. The concentrated product was purified using a silica gel column chromatography, obtaining 35.3 g (82%) of a product Da (phenyl, GC Mass (M+H+)=398.08, 400.06).

### Synthesis of Intermediate Db

30.1 g of an intermediate Db was finally prepared according to the same method of preparing the intermediate Da except for using the intermediate Ab.

### Synthesis of Intermediate Dc

27.3 g of an intermediate Dc was finally prepared according to the same method of preparing the intermediate Da except for using the intermediate Ac.

### Synthesis of Intermediate Dd

27.1 g of an intermediate Dd was finally prepared according to the same method of preparing the intermediate Da except for using the intermediate Ad.

An intermediate F was synthesized according to the following Reaction Scheme 2.

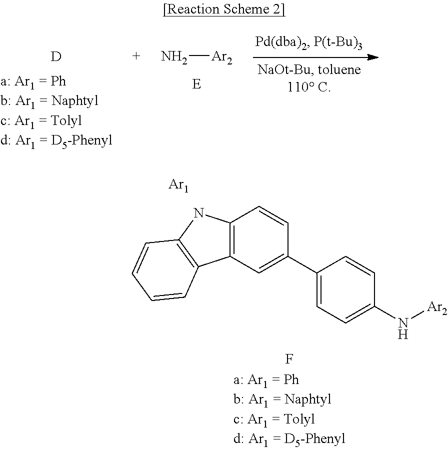

### Synthesis of Intermediate F

15.0 g of the intermediate D and 1.5 equivalent of an aryl amine E sodium, 1.1 equivalent of tertbutoxide, 0.02 equivalent of Pd(dba)2[(tris(dibenzylidine acetone)dipalladium (0))], and 0.02 equivalent of tri(tert-butyl)phosphine based on the amount of the intermediate D were dissolved in 200 mL of toluene. The solution was reacted for 12 hours at 110° C. in a 250 ml 3-necked round-bottomed flask. When the reaction was complete, the reaction mixture was cooled down to room temperature, and 100 ml of distilled water was added thereto. Then an organic layer was extracted. The organic layer was collected and dried and concentrated with MgSO₄ through a silica gel column chromatography. Then, an obtained elution solution was concentrated and dried, obtaining a desired solid compound, which was identified using LCMS.

The following Table 1 provides kinds of products obtained using an intermediate D and an aryl amine F according to Examples.

TABLE 1

| | | | | Yield |
|---|---|---|---|---|
| | | | | of |
| | | | | pro- |
| | | | | duct |
| | Ar1 of | | Structure | F |
| No. | intermediate D | Ar2 of arylamine E | of intermediate F | (%) |
| F1 | 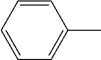 | 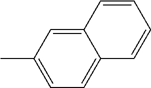 | 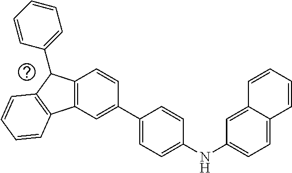 [SMILES: c1ccc(C2c3ccccc3-c3cc(-c4ccc(Nc5ccc6ccccc6c5)cc4)ccc32)cc1] | 68 |
| F2 | 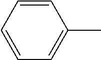 | 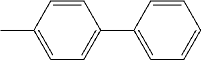 | 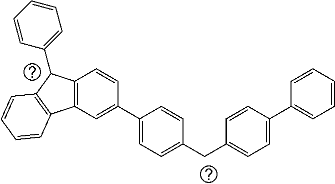 [SMILES: c1ccc(-c2ccc(Cc3ccc(-c4ccc5c(c4)-c4ccccc4C5c4ccccc4)cc3)cc2)cc1] | 73 |
| F3 | 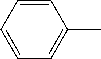 | 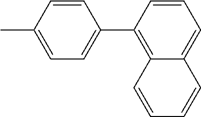 [SMILES: Cc1ccc(-c2cccc3ccccc23)cc1] | 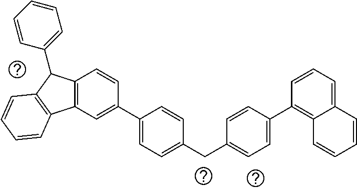 [SMILES: c1ccc(C2c3ccccc3-c3cc(-c4ccc(Cc5ccc(-c6cccc7ccccc67)cc5)cc4)ccc32)cc1] | 62 |
| F4 | 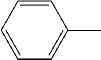 | 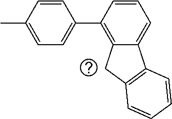 [SMILES: Cc1ccc(-c2cccc3c2Cc2ccccc2-3)cc1] | 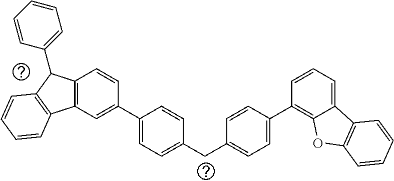 [SMILES: c1ccc(C2c3ccccc3-c3cc(-c4ccc(Cc5ccc(-c6cccc7c6oc6ccccc67)cc5)cc4)ccc32)cc1] | 80 |
| F5 | 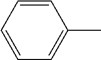 | 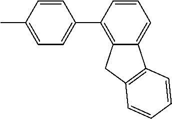 [SMILES: Cc1ccc(-c2cccc3c2Cc2ccccc2-3)cc1] | 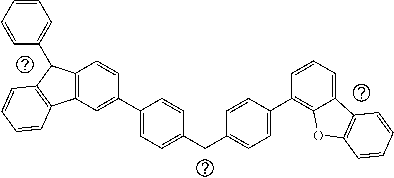 [SMILES: c1ccc(C2c3ccccc3-c3cc(-c4ccc(Cc5ccc(-c6cccc7c6oc6ccccc67)cc5)cc4)ccc32)cc1] | 85 |
| F6 | 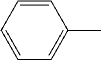 | 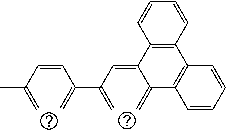 [SMILES: C=C(C)/C=C\C(=C)C(=C)/C=c1\c(=C)c2ccccc2c2ccccc12] | 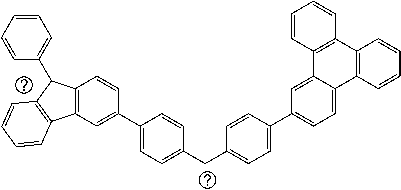 [SMILES: c1ccc(C2c3ccccc3-c3cc(-c4ccc(Cc5ccc(-c6ccc7c8ccccc8c8ccccc8c7c6)cc5)cc4)ccc32)cc1] | 88 |
| F7 | 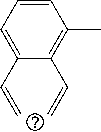 | 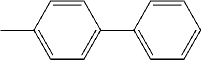 | 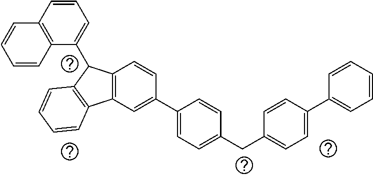 [SMILES: c1ccc(-c2ccc(Cc3ccc(-c4ccc5c(c4)-c4ccccc4C5c4cccc5ccccc45)cc3)cc2)cc1] | 92 |
| F8 | 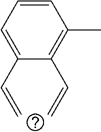 | 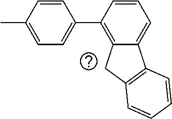 [SMILES: Cc1ccc(-c2cccc3c2Cc2ccccc2-3)cc1] | 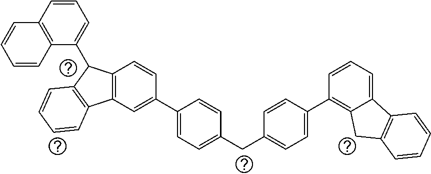 [SMILES: c1ccc2c(c1)Cc1c(-c3ccc(Cc4ccc(-c5ccc6c(c5)-c5ccccc5C6c5cccc6ccccc56)cc4)cc3)cccc1-2] | 88 |
| F9 | 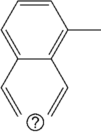 | 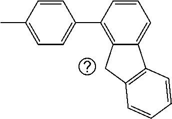 [SMILES: Cc1ccc(-c2cccc3c2Cc2ccccc2-3)cc1] | 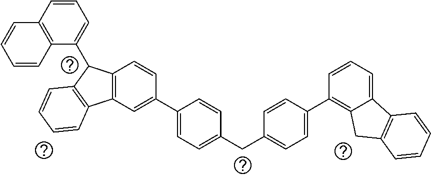 [SMILES: c1ccc2c(c1)Cc1c(-c3ccc(Cc4ccc(-c5ccc6c(c5)-c5ccccc5C6c5cccc6ccccc56)cc4)cc3)cccc1-2] | 85 |
| F10 | 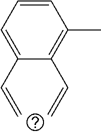 | 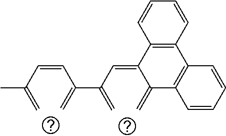 [SMILES: C=C(C)/C=C\C(=C)C(=C)/C=c1\c(=C)c2ccccc2c2ccccc12] | 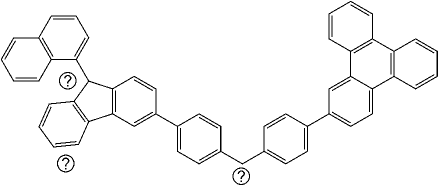 [SMILES: c1ccc2c(c1)-c1cc(-c3ccc(Cc4ccc(-c5ccc6c7ccccc7c7ccccc7c6c5)cc4)cc3)ccc1C2c1cccc2ccccc12] | 90 |
| F11 | 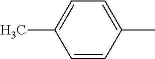 | 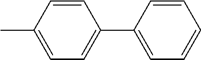 | 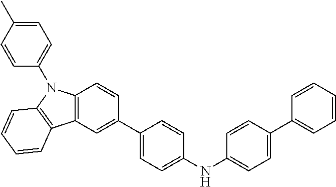 [SMILES: Cc1ccc(-n2c3ccccc3c3cc(-c4ccc(Nc5ccc(-c6ccccc6)cc5)cc4)ccc32)cc1] | 81 |
| F12 | 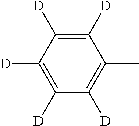 | 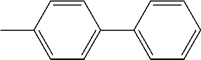 | 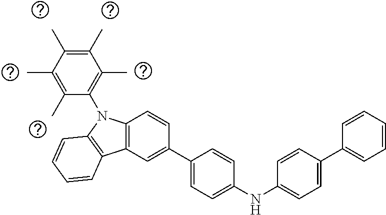 [SMILES: Cc1c(C)c(C)c(-n2c3ccccc3c3cc(-c4ccc(Nc5ccc(-c6ccccc6)cc5)cc4)ccc32)c(C)c1C] | 79 |

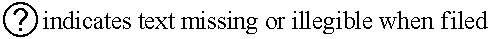

### Synthesis of Intermediates G and H

The following Reaction Scheme 3 shows a method of synthesizing an intermediate H.

The intermediate H uses the intermediate G as a reactant for the synthesis in a method described in Tetrahedron Letters, 38, 6367, 1997.

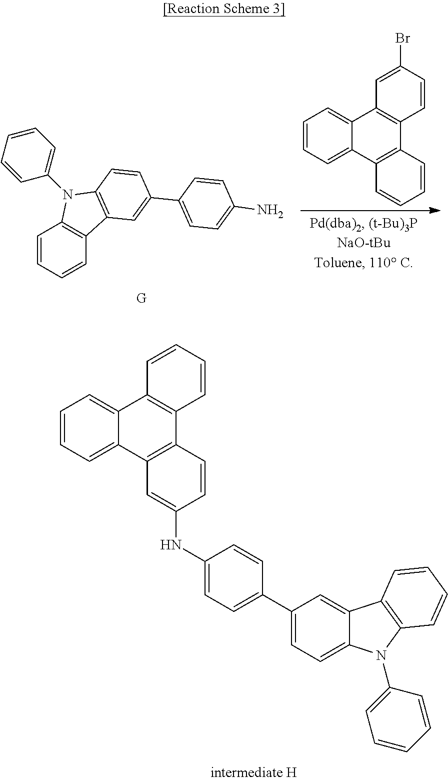 [SMILES: Nc1ccc(-c2ccc3c(c2)c2ccccc2n3-c2ccccc2)cc1 ; Brc1ccc2c3ccccc3c3ccccc3c2c1 ; c1ccc(-n2c3ccccc3c3cc(-c4ccc(Nc5ccc6c7ccccc7c7ccccc7c6c5)cc4)ccc32)cc1]

11.0 g (30.0 mmol) of the intermediate G in the Reaction Scheme 3 and 8.8 g (28.5 mmol) of triphenylene bromide were put in a 250 ml 2-necked round-bottomed flask, and 500 mL of toluene was filled in to dissolve the reactants. 3.2 g of sodium tertbutoxide, 0.518 g of Pd(dba)₂[(tris(dibenzylidine acetone)dipalladium (0))], and 0.364 g of tri(tert-butyl)phosphine were sequentially put in a reactor and reacted for 12 hours at 110° C. When the reaction was complete, the reaction mixture was cooled down to room temperature, and 100 ml of distilled water was added thereto, extracting an organic layer. The organic layer was collected, dried and concentrated with MgSO₄, and treated through a silica gel column chromatography. The obtained eluted solution was concentrated and dried, a desired solid compound, which was identified using LCMS.

### Example 1

### Synthesis of Compound Represented by Chemical Formula 4

A compound represented by the above Chemical Formula 4 was synthesized through the following Reaction Scheme 4.

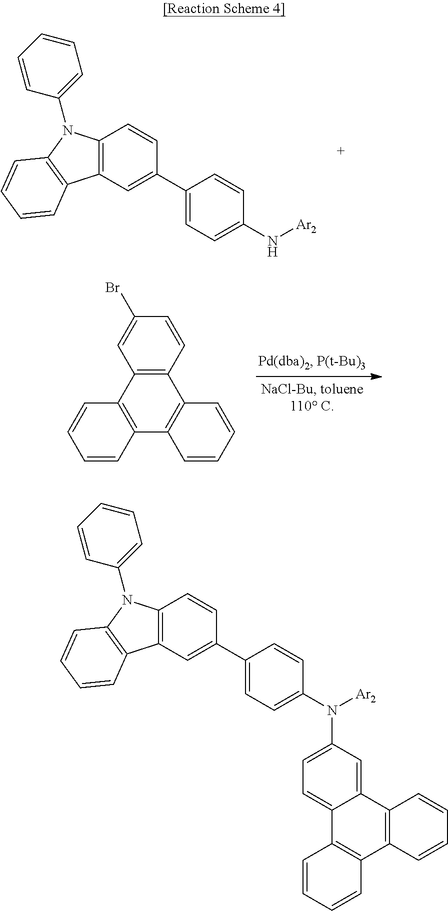

6.45 g (14.0 mmol) of the intermediate F1 provided in Table 1 and 4.73 g (15.4 mmol) of triphenylene bromide were put in a 250 ml 2-necked round-bottomed flask, and 150 mL of toluene was filled in the flask to dissolve the reactants. Then, 1.40 g of sodium tertbutoxide, 0.228 g of Pd(dba)₂[(tris(dibenzylidine acetone)dipalladium (0))], and 0.16 g of tri(tert-butyl)phosphine were put in a reactor and reacted for 12 hours at 110° C. When the reaction was complete, the reaction mixture was cooled down to room temperature, and 100 ml of distilled water was added thereto to extract an organic layer. The organic layer was dried and concentrated with MgSO₄ and then treated through a silica gel column chromatography. The obtained eluted solution was concentrated and dried, obtaining a desired solid compound [M+H+=687.28], which was identified using LCMS.

### Example 2

### Synthesis of Compound Represented by Chemical Formula 9

A compound represented by Chemical Formula 9 was prepared according to the same method as Example 1 except for using the intermediate F2 in Table 1 and identified using LCMS [M+H+=713.30].

### Example 3

### Synthesis of Compound Represented by Chemical Formula 60

A compound represented by Chemical Formula 60 was prepared according to the same method as Example 1 except for using the intermediate F4 in Table 1 and identified using LCMS [M+H+=804.31].

### Example 41

### Synthesis of Compound Represented by Chemical Formula 29

A compound represented by Chemical Formula 29 was prepared according to the same method as Example 1 except for using the intermediate F6 in Table 1 and identified using LCMS [M+H+=863.34].

### Example 42

### Synthesis of Compound Represented by Chemical Formula K1

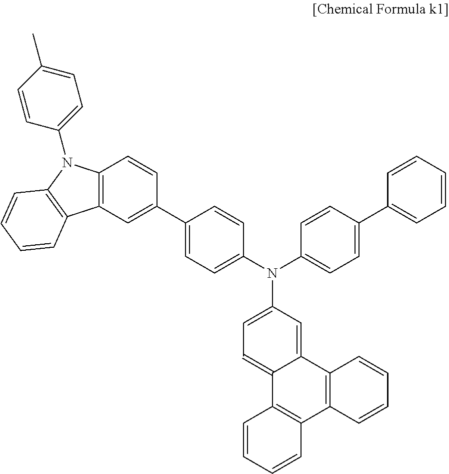 [SMILES: Cc1ccc(-n2c3ccccc3c3cc(-c4ccc(N(c5ccc(-c6ccccc6)cc5)c5ccc6c7ccccc7c7ccccc7c6c5)cc4)ccc32)cc1]

A compound represented by Chemical Formula k1 was prepared according to the same method as Example 1 except for using the intermediate F11 in Table 1 and identified using LCMS [M+H+=727.37].

### Example 43

### Synthesis of Compound Represented by Chemical Formula K2

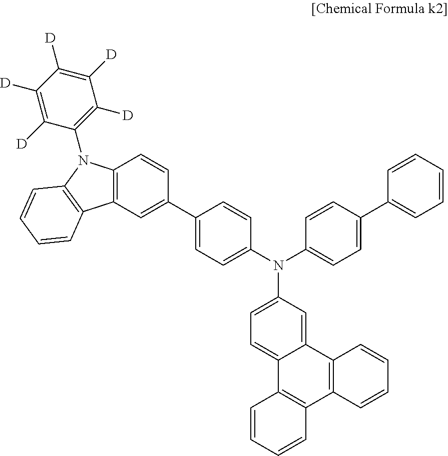 [SMILES: [2H]c1c([2H])c([2H])c(-n2c3ccccc3c3cc(-c4ccc(N(c5ccc(-c6ccccc6)cc5)c5ccc6c7ccccc7c7ccccc7c6c5)cc4)ccc32)c([2H])c1[2H]]

A compound represented by the above Chemical Formula was prepared according to the same method as Example 1 except for using the intermediate F12 in Table 1 and identified using LCMS [M+H+=717.42].

### Example 44

### Synthesis of Compound Represented by Chemical Formula 107

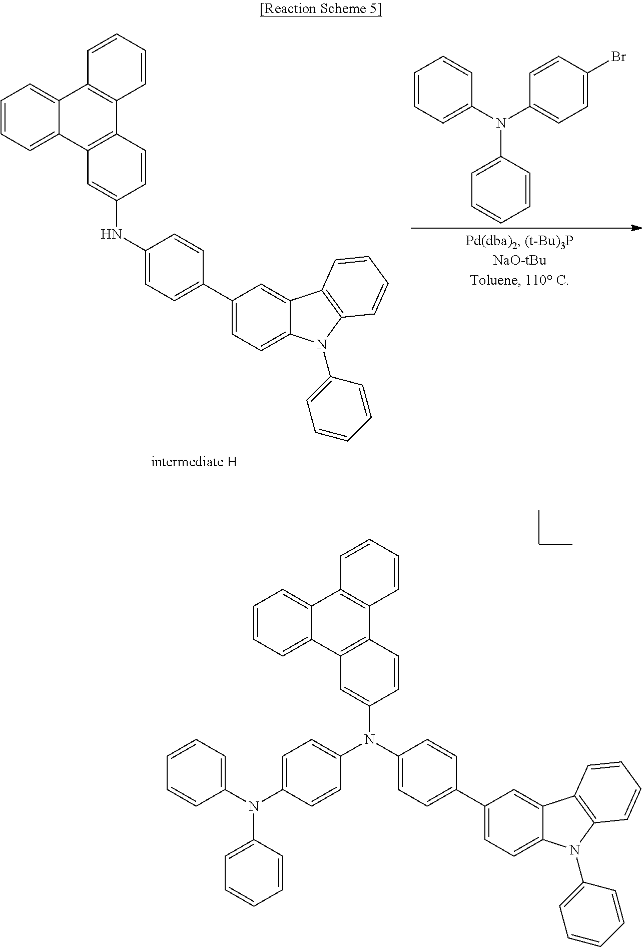 [SMILES: c1ccc(-n2c3ccccc3c3cc(-c4ccc(Nc5ccc6c7ccccc7c7ccccc7c6c5)cc4)ccc32)cc1 ; Brc1ccc(N(c2ccccc2)c2ccccc2)cc1 ; c1ccc(N(c2ccccc2)c2ccc(N(c3ccc(-c4ccc5c(c4)c4ccccc4n5-c4ccccc4)cc3)c3ccc4c5ccccc5c5ccccc5c4c3)cc2)cc1]

8.0 g (14.3 mmol) of the intermediate H provided in Reaction Scheme 3 and 5.6 g (5.6 mmol) of triphenylene bromide were put in a 250 ml 2-necked round-bottomed flask, and 250 mL of toluene was filled therein to dissolve the reactants. Then, 1.5 g of sodium tertbutoxide, 0.246 g of Pd(dba)₂[(tris(dibenzylidine acetone)dipalladium (0))], and 0.173 g of tri(tert-butyl)phosphine were sequentially put in a reactor and reacted for 12 hours at 110° C. When the reaction was complete, the reaction mixture was cooled down to room temperature, and 100 ml of distilled water was added thereto to extract an organic layer. The organic layer was dried and concentrated with MgSO₄ and then treated through a silica gel column chromatography. The obtained elution solution was concentrated and dried, obtaining a desired solid compound [M+H+=804.34] and identified using LCMS.

Fabrication of Organic Light Emitting Diode

### Example 5

### Fabrication of Organic Light Emitting Diode Including a Hole Transport Layer (HTL) Made of the Compound According to Example 2

As for an anode, a 15 Ωcm 1200 Å ITO glass substrate (Corning Inc.) was cut to have a size of 50 mm×50 mm×0.7 mm, ultrasonic wave-washed with isopropyl alcohol and pure water for 5 minutes, radiated with ultraviolet (UV) light for 30 minutes, and mounted in a vacuum deposition device.

Next, the following 2-TNATA was vacuum-deposited to form a 600 Å-thick hole injection layer (HIL) on the substrate, and the compound according to Example 2 was vacuum deposited to form a 300 Å-thick hole transport layer (HTL).

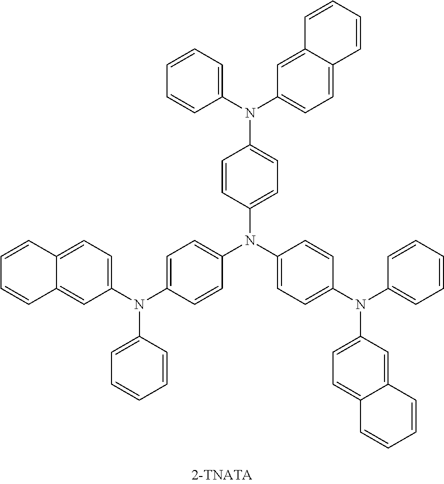 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

On the hole transport layer (HTL), a blue fluorescent host IDE215 (Idemitsu Co. Ltd.) and a blue fluorescent dopant IDE118 (Idemitsu Co. Ltd.) in a weight ratio of 98:2 were simultaneously deposited to be a 200 Å thick emission layer.

Next, an electron transport layer (ETL) was formed by depositing Alq3 to be 300 Å thick on the emission layer, an electron injection layer (EIL) was deposited to be 10 Å thick thereon by depositing a halogenated alkali metal, LiF, and a LiF/Al electrode (cathode electrode) was formed by vacuum-depositing Al to be 3000 Å thick, fabricating an organic light emitting diode. The organic light emitting diode had a driving voltage of 4.7 V, current density of 14.9 mA/cm², a color coordinate (0.133, 0.140), and luminous efficiency of 6.7 cd/A at a light emitting luminance of 1000 nit.

### Example 6

### Fabrication of Organic Light Emitting Diode Including Hole Transport Layer (HTL) Formed of the Compound According to Example 3

An organic light emitting diode was fabricated according to the same method as Example 5 except for using the compound according to Example 3 instead of the compound according to Example 2 to form the hole transport layer (HTL). This organic light emitting diode had a driving voltage of 4.7 V, current density of 15.9 mA/cm², a color coordinate (0.133, 0.139), and luminous efficiency of 6.3 cd/A at a light emitting luminance of 1000 nit.

### Example 7

### Fabrication of Organic Light Emitting Diode Including Hole Transport Layer (HTL) Formed of the Compound of Example 41

An organic light emitting diode was fabricated according to the same method as Example 5 except for using the compound of Example 4 instead of the compound of Example 2 to form a hole transport layer (HTL). This organic light emitting diode had a driving voltage of 4.8 V, current density of 16.3 mA/cm², a color coordinate (0.133, 0.139), and luminous efficiency of 6.2 cd/A at a light emitting luminance of 1000 nit.

### Example 8

### Fabrication of Organic Light Emitting Diode Including Hole Transport Layer (HTL) Formed of the Compound of Example 42

An organic light emitting diode was fabricated according to the same method as Example 5 except for using the compound of Example 42 instead of the compound of Example 2 to form a hole transport layer (HTL). This organic light emitting diode had a driving voltage of 4.9 V, current density of 14.3 mA/cm², color coordinate (0.133, 0.138), and luminous efficiency of 6.2 cd/A at a light emitting luminance of 1000 nit.

### Example 9

### Fabrication of Organic Light Emitting Diode Including Hole Transport Layer (HTL) Formed of the Compound of Example 43

An organic light emitting diode was fabricated according to the same method as Example 5 except for using the compound of Example 43 instead of the compound of Example 2 to form a hole transport layer (HTL). This organic light emitting diode had a driving voltage of 4.7 V, current density of 14.9 mA/cm², color coordinate (0.133, 0.138), and luminous efficiency of 6.8 cd/A at a light emitting luminance of 1000 nit.

### Example 10

### Fabrication of Organic Light Emitting Diode Including Hole Transport Layer (HTL) Formed of the Compound of Example 44

An organic light emitting diode was fabricated according to the same method as Example 5 except for using the compound of Example 44 instead of the compound of Example 2 to form a hole transport layer (HTL). The organic light emitting diode had a driving voltage of 4.9 V, current density of 15.3 mA/cm², color coordinate (0.133, 0.138), and luminous efficiency of 6.4 cd/A at a light emitting luminance of 1000 nit.

### Comparative Example 1

### Fabrication of Organic Light Emitting Diode Including Hole Transport Layer (HTL) Formed of NPB

An organic light emitting diode was fabricated according to the same method as Example 5 except for using 4,4′-bis[N-(1-naphthyl)-N-phenylamino]biphenyl (hereinafter, NPB) instead of the compound of Example 2 to form a hole transport layer (HTL). The organic light emitting diode had a driving voltage of 5.5 V, current density of 15.9 mA/cm², a color coordinate (0.133, 0.139), and luminous efficiency of 4.2 cd/A at a light emitting luminance of 1000 nit.

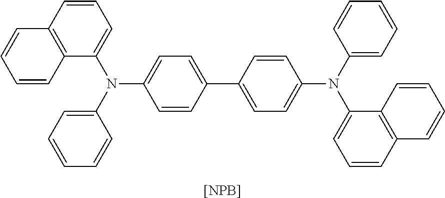

### Comparative Example 2

### Fabrication of Organic Light Emitting Diode Including Hole Transport Layer (HTL) Formed of HT1

An organic light emitting diode was fabricated according to the same method as Example 5 except for using HT1 instead of the compound of Example 2 to form a hole transport layer (HTL). The organic light emitting diode had a driving voltage of 5.0 V, current density of 13.9 mA/cm², a color coordinate (0.133, 0.139), and luminous efficiency of 5.8 cd/A at a light emitting luminance of 1000 nit.

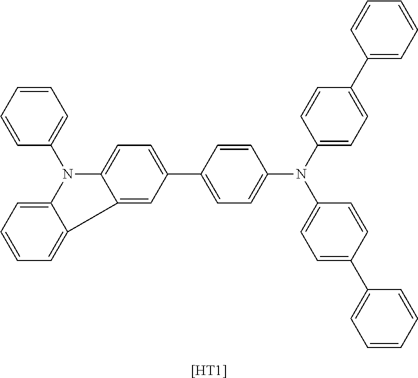

### Comparative Example 3

### Fabrication of Organic Light Emitting Diode Including Hole Transport Layer (HTL) Formed of HT2

An organic light emitting diode was fabricated according to the same method as Example 5 except for using HT2 instead of the compound of Example 2 to form a hole transport layer (HTL). This organic light emitting diode had a driving voltage of 4.9 V, current density of 12.9 mA/cm², a color coordinate (0.133, 0.138), luminous efficiency of 5.9 cd/A at a light emitting luminance of 1000 nit.

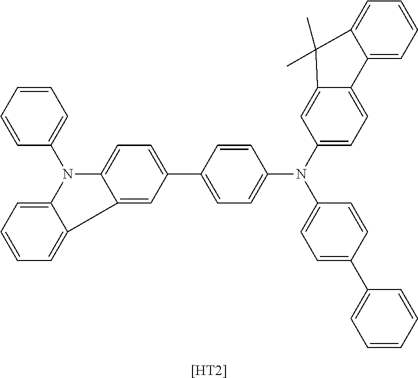

Thermal Stability of Compound

The compounds were measured regarding a glass transition temperature through a secondary scan using DSC 1 (METTLER-TOLEDO Inc.) and increasing their temperatures up to 320° C. by 10° C./min and regarding thermal decomposition temperature by increasing their temperature up to 900° C. by 10° C./min under a nitrogen atmosphere and measuring an onset point temperature.

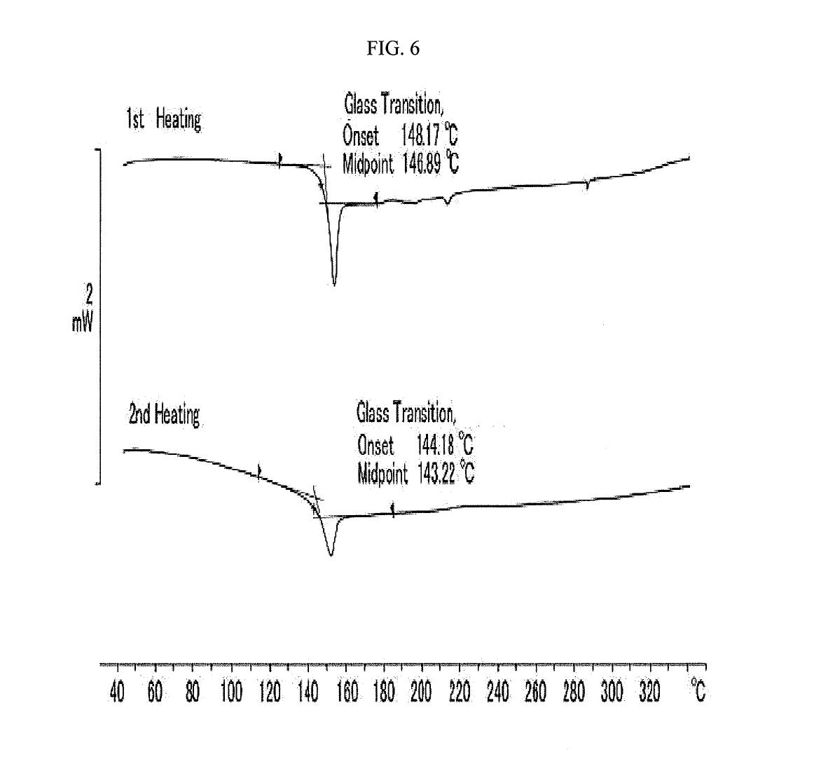

Herein, the compound of Example 2 had a glass transition temperature of 143° C. The results are provided in FIG. 6.

The aforementioned transition temperature is considered high enough to be used for an organic photoelectric device according to the influence of a glass transition temperature on life-span of an organic photoelectric device as set forth in an article by Adachi et al., Appl. Phys. Lett. 51, 913 1990.

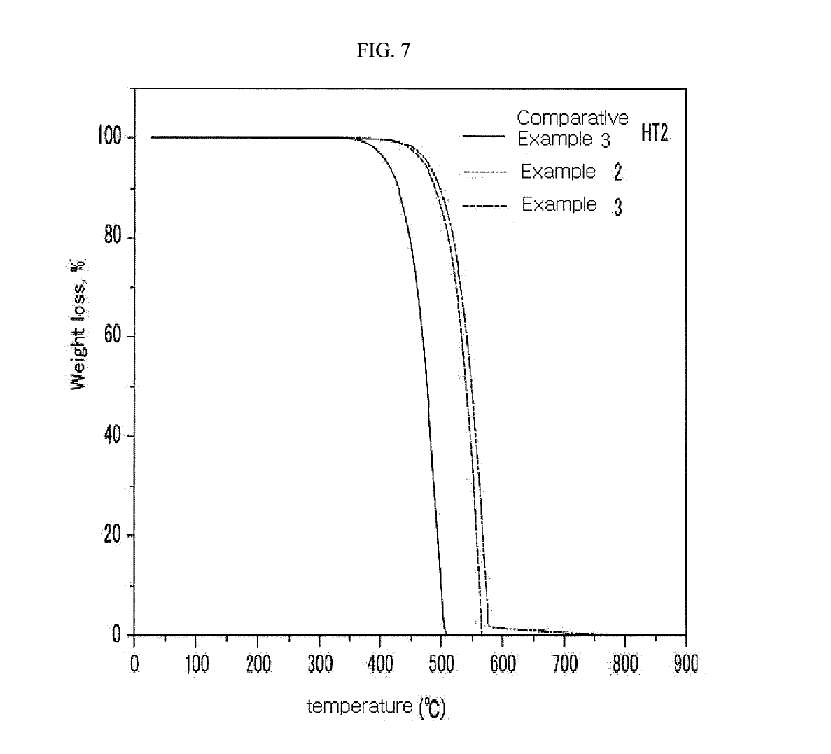

The HT2 compound of Comparative Example 3 and the compounds according to Examples 2 and 3 had a thermal decomposition temperature of respectively 449° C., 525° C., and 522° C. The results are provided in FIG. 7.

In other words, the compound according to Examples 2 and 3 had remarkably higher thermal stability than the HT2 compound according to Comparative Example 3.

Performance of Organic Light Emitting Diode

Each organic light emitting diode according to Examples 5 to 10 and Comparative Examples 1 to 3 was measured regarding current density and luminance changes depending on voltage and luminous efficiency. Specific measurement methods were as follows, and the results are shown in the following Table 2.

(1) Measurement of Current Density Change Depending on Voltage Change

The fabricated organic light emitting diodes were measured for current value flowing in the unit device while increasing the voltage from 0 V to 10 V using a current-voltage meter (Keithley 2400), and the measured current value was divided by area to provide the result.

(2) Measurement of Luminance Change Depending on Voltage Change

The fabricated organic light emitting diodes were measured for luminance while increasing the voltage from 0 V to 10 V using a luminance meter (Minolta Cs1000A).

(3) Measurement of Luminous Efficiency

Current efficiency (cd/A) and electric power efficiency (lm/W) at the same luminance (1000 cd/m²) were calculated by using luminance and current density from the item (1) and (2) and voltage.

(4) Color Coordinate was Measured Using Luminance Meter (Minolta Cs100A).

TABLE 2

| | 1000 cd/m² | | | |
|---|---|---|---|---|
| | | Current | Luminous | |
| | Driving voltage | density | efficiency | Color |
| | (V) | (mA/cm²) | (cd/A) | coordinate |
| Example 5 | 4.7 | 14.9 | 6.7 | (0.133, 0.140) |
| Example 6 | 4.7 | 15.9 | 6.3 | (0.133, 0.139) |
| Example 7 | 4.8 | 16.3 | 6.2 | (0.133, 0.139) |
| Example 8 | 4.9 | 14.3 | 6.2 | (0.133, 0.138) |
| Example 9 | 4.7 | 14.9 | 6.8 | (0.133, 0.138) |
| Example 10 | 4.9 | 15.3 | 6.4 | (0.133, 0.138) |
| Comparative | 5.5 | 15.9 | 4.2 | (0.133, 0.139) |
| Example 1 | | | | |
| Comparative | 5.0 | 13.9 | 5.8 | (0.133, 0.139) |
| Example 2 | | | | |
| Comparative | 4.9 | 12.9 | 5.9 | (0.133, 0.139) |
| Example 3 | | | | |

As shown in Table 2, it is confirmed that the organic light emitting diodes according to Examples 5 to 10 had lower driving voltages and better luminous efficiency and electric power efficiency than those of Comparative Example 1. The compounds according to the Examples had excellent hole injection and hole transport capabilities and may be used to provide an organic light emitting diode that may exhibit low voltage, high efficiency, high luminance, and a long life-span.

By way of summation and review, examples of an organic photoelectric device may include an organic light emitting diode, an organic solar cell, an organic photo conductor drum, and an organic transistor, and the like. Such devices may use a hole injecting or transport material, an electron injecting or transport material, or a light emitting material.

An organic light emitting diode may convert electrical energy into light by applying current to an organic light emitting material, and may have a structure in which a functional organic material layer is interposed between an anode and a cathode. The organic material layer may be configured as a multi-layer including different materials, for example a hole injection layer (HIL), a hole transport layer (HTL), an emission layer, an electron transport layer (ETL), and an electron injection layer (EIL), in order to enhance efficiency and stability of an organic photoelectric device. In such an organic light emitting diode, when a voltage is applied between an anode and a cathode, holes from the anode and electrons from the cathode may be injected to an organic material layer and recombined to generate excitons having high energy, which may generate light having certain wavelengths while shifting to a ground state. A phosphorescent light emitting material may be used for a light emitting material of an organic light emitting diode, in addition to a fluorescent light emitting material. A phosphorescent material may emit light by transporting electrons from a ground state to an exited state, non-radiance transiting of a singlet exciton to a triplet exciton through intersystem crossing, and transiting a triplet exciton to a ground state to emit light.

In an organic light emitting diode, an organic material layer may include a light emitting material and a charge transport material, e.g., a hole injection material, a hole transport material, an electron transport material, an electron injection material, and the like. The light emitting material may be classified as blue, green, and red light emitting materials according to emitted colors, and yellow and orange light emitting materials to emit colors approaching natural colors. When one material is used as a light emitting material, a maximum light emitting wavelength may be shifted to a long wavelength or color purity may decrease because of interactions between molecules, or device efficiency may decrease because of a light emitting quenching effect. Therefore, a host/dopant system may be included as a light emitting material in order to enhance color purity and increase luminous efficiency and stability through energy transfer. A material constituting an organic material layer, e.g., a hole injection material, a hole transport material, a light emitting material, an electron transport material, an electron injection material, and a light emitting material such as a host and/or a dopant, that is stable and has good efficiency may enhance performance of an organic light emitting diode.

A low molecular organic light emitting diode may be manufactured as a thin film in a vacuum deposition method and may exhibit good efficiency and life-span performance. A polymeric organic light emitting diode may manufactured in an inkjet or spin coating method, which may help lower initial costs and enable fabrication of large-sized displays.

Both low molecular organic light emitting and polymeric organic light emitting diodes may be self-light emitting and may provide a display with a high speed response, wide viewing angle, reduced thickness, high image quality, durability, large driving temperature range, and the like. Both low molecular organic light emitting and polymeric organic light emitting diodes may provide a display that has good visibility due to a self-light emitting characteristic, as compared with an LCD (liquid crystal display), and decrease display thickness and weight, relative to the LCD, up to a third by omitting a backlight. In addition, the displays may have a response speed of a microsecond unit, which may be 1000 times faster than an LCD, and they may help to provide a perfect motion picture without an after-image. The displays have been developed to have enhanced characteristics, e.g., 80 times the efficiency and more than 100 times the life-span. The displays have been increased in size, such as a 40-inch organic light emitting diode panel.

Enhanced luminous efficiency and life-span are desirable. Luminous efficiency may be enhanced by smooth combination between holes and electrons in an emission layer. An organic material may have slower electron mobility than hole mobility, which may reduce efficiency of combination between holes and electrons. Accordingly, increasing electron injection and mobility from a cathode and simultaneously preventing movement of holes may improve efficiency. Reducing material crystallization, which may be caused by Joule heating generated during device operation, may enhance life-span. Accordingly, characteristics of a device may be enhanced by using an organic compound having excellent electron injection and mobility, and high electrochemical stability.

As described above, a compound according to embodiments may be used for an organic photoelectric device, which may excellent electrochemical and thermal stability and life-span characteristics, and high luminous efficiency at a low driving voltage. A compound according to embodiments may be used as a light emitting material, an electron injection and/or transport material, a light emitting host along with a dopant, etc.

Description of symbols: 100: organic light emitting diode; 110: cathode; 120: anode; 105: organic thin layer; 130: emission layer; 140: hole transport layer (HTL); 150: electron transport layer (ETL); 160: electron injection layer (EIL); 170: hole injection layer (HIL); 230: emission layer+electron transport layer (ETL)

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic photoelectric device, the compound being represented by the following Chemical Formula 1: 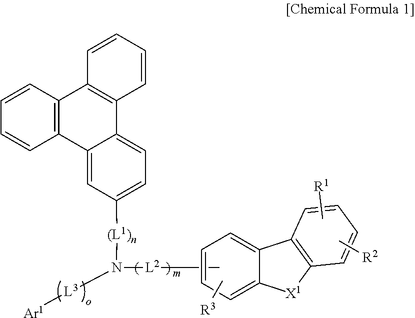
wherein, in Chemical Formula 1,
L¹ to L³ are each independently selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,
n, m, and o are each independently integers ranging from 1 to 4,
X¹ is selected from the group of NR′, O, S, and P, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group,
Ar¹ is a substituted or unsubstituted C6 to C30 aryl group or a substituted or unsubstituted C2 to C30 heteroaryl group, and
R¹ to R³ are each independently selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

2. The compound as claimed in claim 1, wherein X¹ is NR′, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

3. The compound as claimed in claim 1, wherein Ar¹ is selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group.

4. A compound for an organic photoelectric device, the compound being represented by the following Chemical Formula 2: 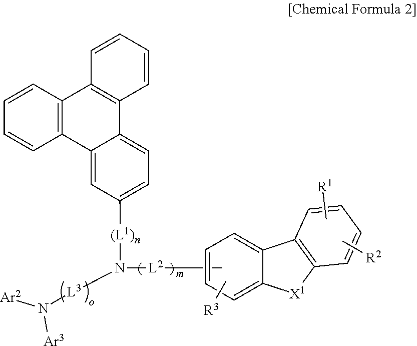
wherein, in Chemical Formula 2,
L¹ to L³ are each independently selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,
n, m, and o are each independently integers ranging from 1 to 4,
X¹ is selected from the group of NR′, O, S, and P, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group,
Ar² and Ar^{a} are each independently selected from the group of a substituted or unsubstituted C1 to C6 alkyl group, a substituted or unsubstituted C6 to C30 aryl group, and a substituted or unsubstituted C2 to C30 heteroaryl group, and
R¹ to R³ are each independently selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

5. The compound as claimed in claim 4, wherein X¹ is NR′, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group

6. The compound as claimed in claim 4, wherein the Ar² and Ar³ are each independently selected from the group of a phenyl group, a naphthyl group, an anthracenyl group, a phenanthryl group, a naphthacenyl group, a pyrenyl group, a biphenylyl group, a p-terphenyl group, a m-terphenyl group, a chrysenyl group, a triphenylenyl group, a perylenyl group, an indenyl group, a furanyl group, a thiophenyl group, a pyrrolyl group, a pyrazolyl group, an imidazolyl group, a triazolyl group, an oxazolyl group, a thiazolyl group, an oxadiazolyl group, a thiadiazolyl group, a pyridyl group, a pyrimidinyl group, a pyrazinyl group, a triazinyl group, a benzofuranyl group, a benzothiophenyl group, a benzimidazolyl group, an indolyl group, a quinolinyl group, an isoquinolinyl group, a quinazolinyl group, a quinoxalinyl group, a naphthyridinyl group, a benzoxazinyl group, a benzthiazinyl group, an acridinyl group, a phenazinyl group, a phenothiazinyl group, and a phenoxazinyl group.

7. A compound for an organic photoelectric device, the compound being represented by the following Chemical Formula 3: 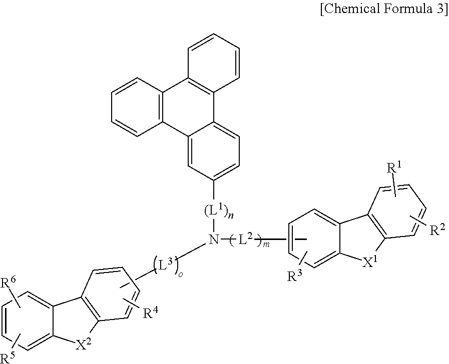
wherein, in Chemical Formula 3,
L¹ to L³ are each independently selected from the group of a single bond, a substituted or unsubstituted C2 to C6 alkenylene group, a substituted or unsubstituted C2 to C6 alkynylene group, a substituted or unsubstituted C6 to C30 arylene group, and a substituted or unsubstituted C2 to C30 heteroarylene group,
n, m, and o are each independently integers ranging from 1 to 4,
X¹ and X² are each independently selected from the group of NR′, O, S, and P, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group, and
R¹ to R⁶ are each independently selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

8. The compound as claimed in claim 7, wherein X¹ and X² are NR′, and R′ is selected from the group of hydrogen, deuterium, a substituted or unsubstituted C1 to C6 alkyl group, a substituted or unsubstituted C6 to C30 aryl group, and a substituted or unsubstituted C2 to C30 heteroaryl group.

9. A compound for an organic photoelectric device, the compound being represented by one of the following Chemical Formulae 4 to 39, ad1, ad2, k1, or k2:

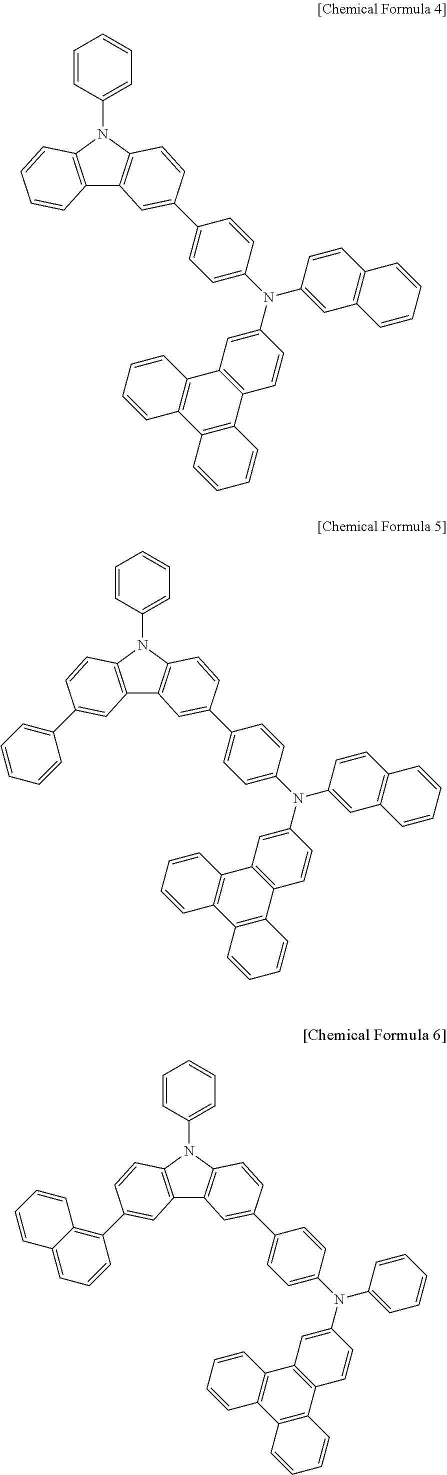 [SMILES: c1ccc(-n2c3ccccc3c3cc(-c4ccc(N(c5ccc6ccccc6c5)c5ccc6c7ccccc7c7ccccc7c6c5)cc4)ccc32)cc1 ; c1ccc(-c2ccc3c(c2)c2cc(-c4ccc(N(c5ccc6ccccc6c5)c5ccc6c7ccccc7c7ccccc7c6c5)cc4)ccc2n3-c2ccccc2)cc1 ; c1ccc(N(c2ccc(-c3ccc4c(c3)c3cc(-c5cccc6ccccc56)ccc3n4-c3ccccc3)cc2)c2ccc3c4ccccc4c4ccccc4c3c2)cc1]

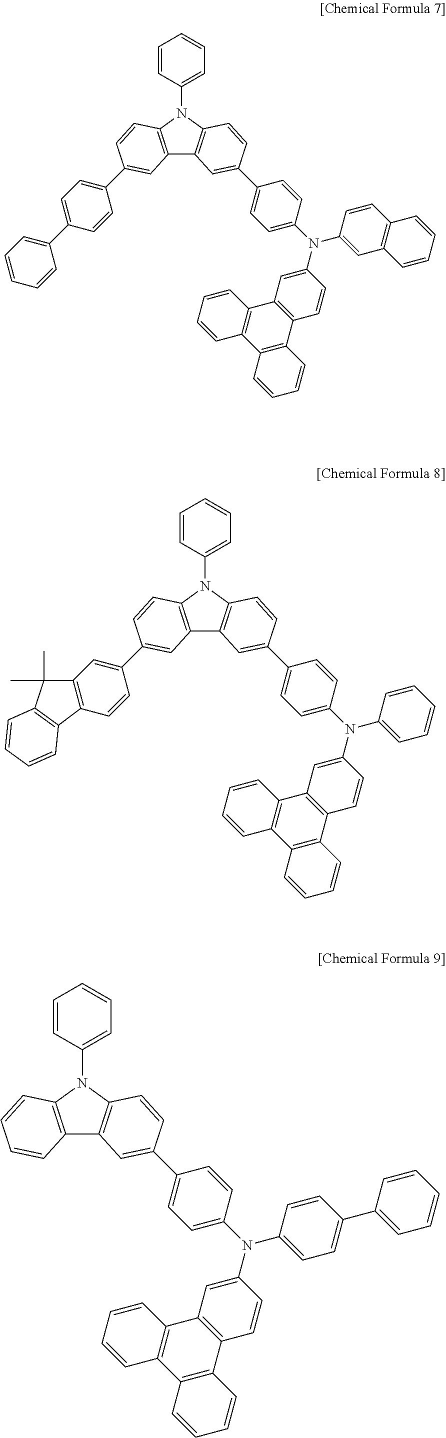 [SMILES: c1ccc(-c2ccc(-c3ccc4c(c3)c3cc(-c5ccc(N(c6ccc7ccccc7c6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccccc6)c6ccc7c8ccccc8c8ccccc8c7c6)cc5)ccc3n4-c3ccccc3)cc21 ; c1ccc(-c2ccc(N(c3ccc(-c4ccc5c(c4)c4ccccc4n5-c4ccccc4)cc3)c3ccc4c5ccccc5c5ccccc5c4c3)cc2)cc1]

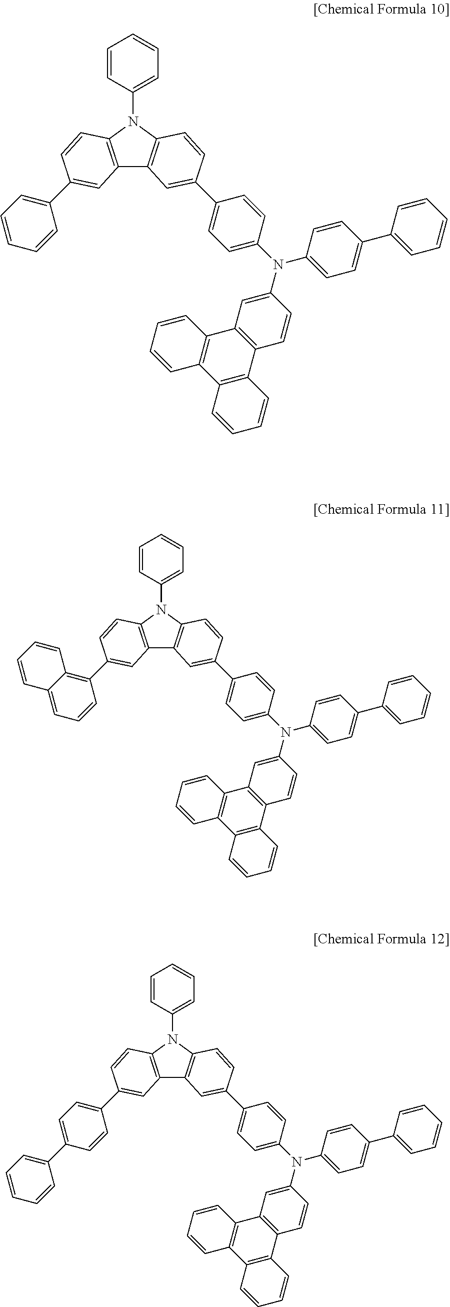 [SMILES: c1ccc(-c2ccc(N(c3ccc(-c4ccc5c(c4)c4cc(-c6ccccc6)ccc4n5-c4ccccc4)cc3)c3ccc4c5ccccc5c5ccccc5c4c3)cc2)cc1 ; c1ccc(-c2ccc(N(c3ccc(-c4ccc5c(c4)c4cc(-c6cccc7ccccc67)ccc4n5-c4ccccc4)cc3)c3ccc4c5ccccc5c5ccccc5c4c3)cc2)cc1 ; c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc2)cc1]

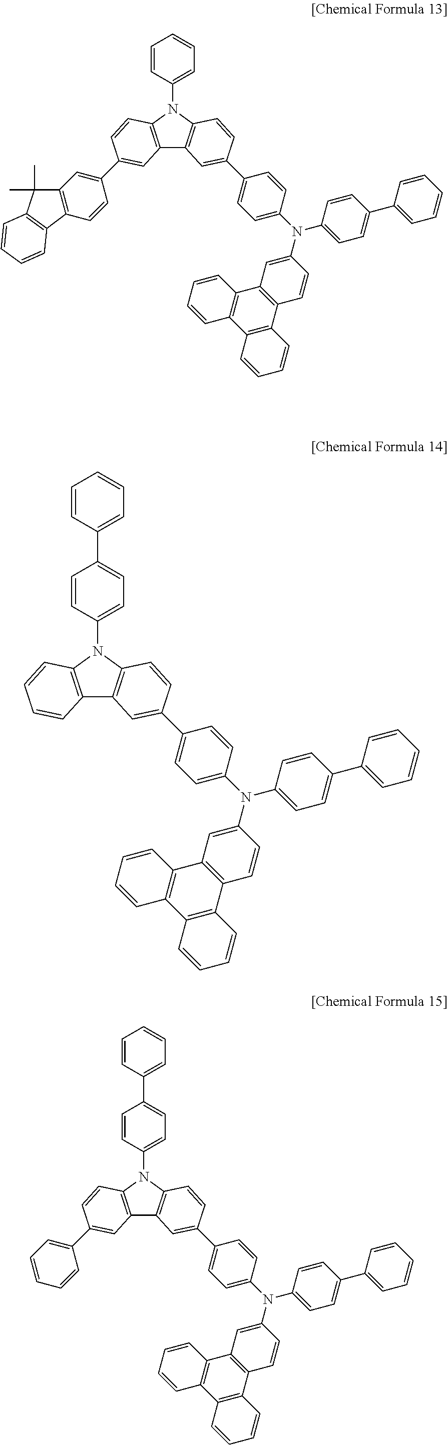 [SMILES: CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccccc3)cc21 ; c1ccc(-c2ccc(N(c3ccc(-c4ccc5c(c4)c4ccccc4n5-c4ccc(-c5ccccc5)cc4)cc3)c3ccc4c5ccccc5c5ccccc5c4c3)cc2)cc1 ; c1ccc(-c2ccc(N(c3ccc(-c4ccc5c(c4)c4cc(-c6ccccc6)ccc4n5-c4ccc(-c5ccccc5)cc4)cc3)c3ccc4c5ccccc5c5ccccc5c4c3)cc2)cc1]

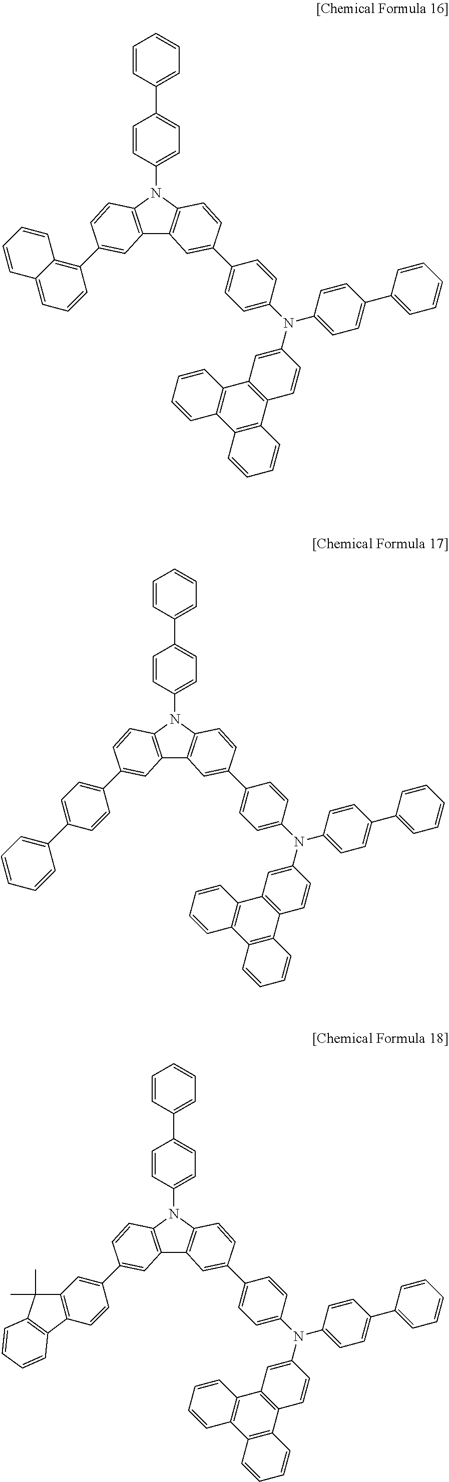 [SMILES: c1ccc(-c2ccc(N(c3ccc(-c4ccc5c(c4)c4cc(-c6cccc7ccccc67)ccc4n5-c4ccc(-c5ccccc5)cc4)cc3)c3ccc4c5ccccc5c5ccccc5c4c3)cc2)cc1 ; c1ccc(-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc(-c7ccccc7)cc6)c6ccc7c8ccccc8c8ccccc8c7c6)cc5)ccc3n4-c3ccc(-c4ccccc4)cc3)cc21]

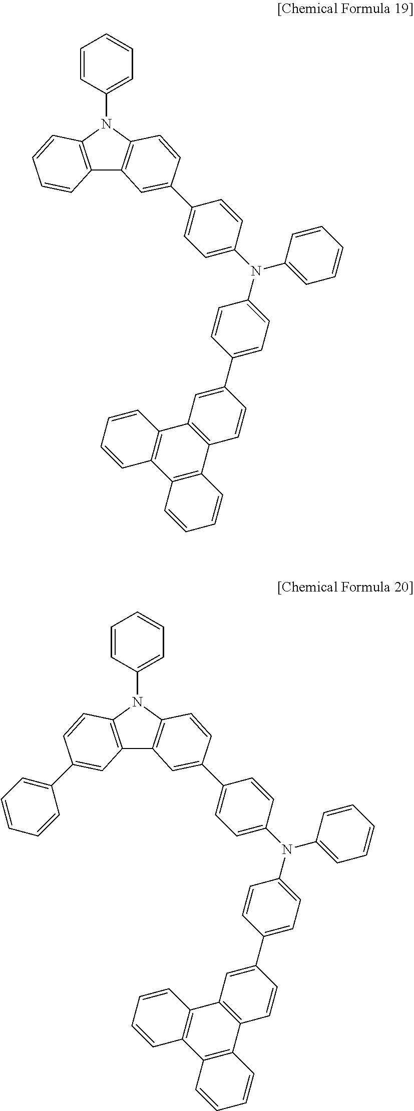 [SMILES: c1ccc(N(c2ccc(-c3ccc4c5ccccc5c5ccccc5c4c3)cc2)c2ccc(-c3ccc4c(c3)c3ccccc3n4-c3ccccc3)cc2)cc1 ; c1ccc(-c2ccc3c(c2)c2cc(-c4ccc(N(c5ccccc5)c5ccc(-c6ccc7c8ccccc8c8ccccc8c7c6)cc5)cc4)ccc2n3-c2ccccc2)cc1]

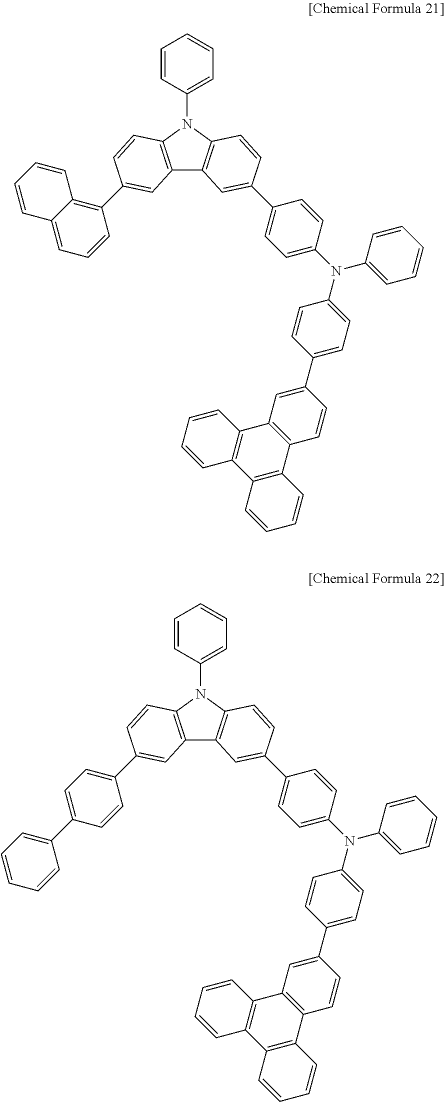 [SMILES: c1ccc(N(c2ccc(-c3ccc4c5ccccc5c5ccccc5c4c3)cc2)c2ccc(-c3ccc4c(c3)c3cc(-c5cccc6ccccc56)ccc3n4-c3ccccc3)cc2)cc1 ; c1ccc(-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc2)cc1]

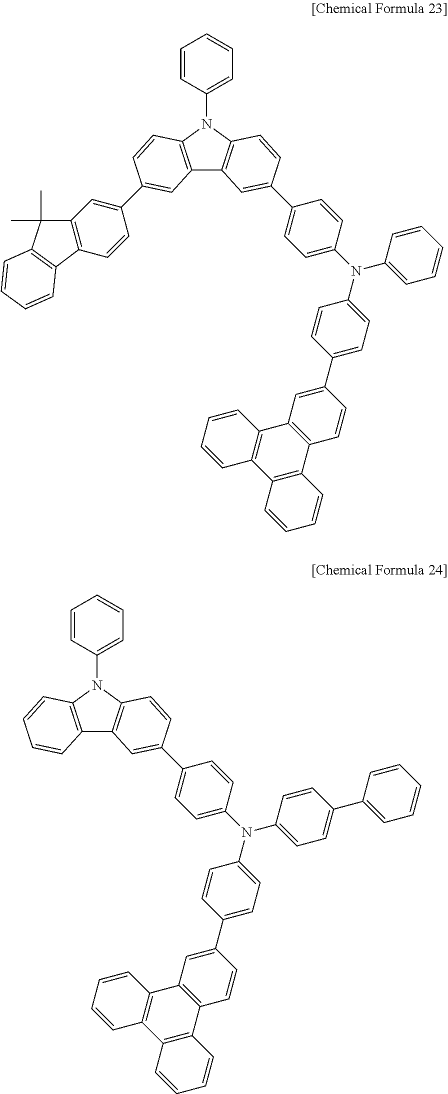 [SMILES: CC1(C)c2ccccc2-c2ccc(-c3ccc4c(c3)c3cc(-c5ccc(N(c6ccccc6)c6ccc(-c7ccc8c9ccccc9c9ccccc9c8c7)cc6)cc5)ccc3n4-c3ccccc3)cc21 ; c1ccc(-c2ccc(N(c3ccc(-c4ccc5c6ccccc6c6ccccc6c5c4)cc3)c3ccc(-c4ccc5c(c4)c4ccccc4n5-c4ccccc4)cc3)cc2)cc1]

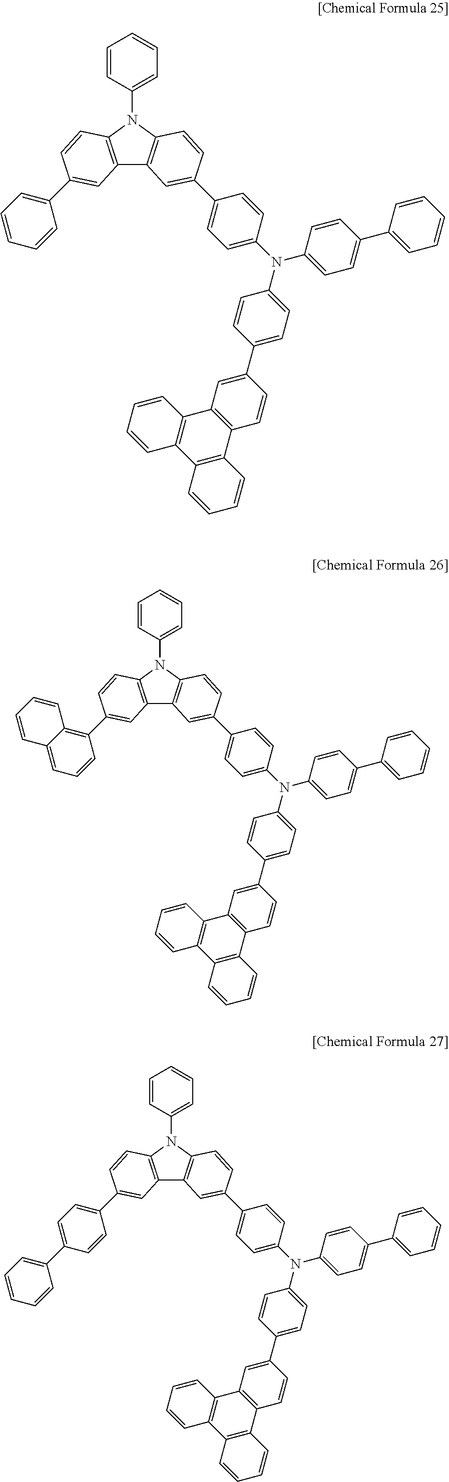 [SMILES: c1ccc(-c2ccc(N(c3ccc(-c4ccc5c6ccccc6c6ccccc6c5c4)cc3)c3ccc(-c4ccc5c(c4)c4cc(-c6ccccc6)ccc4n5-c4ccccc4)cc3)cc2)cc1 ; c1ccc(-c2ccc(N(c3ccc(-c4ccc5c6ccccc6c6ccccc6c5c4)cc3)c3ccc(-c4ccc5c(c4)c4cc(-c6cccc7ccccc67)ccc4n5-c4ccccc4)cc3)cc2)cc1 ; c1ccc(-c2ccc(-c3ccc4c(c3)c3cc(-c5ccc(N(c6ccc(-c7ccccc7)cc6)c6ccc(-c7ccc8c9ccccc9c9ccccc9c8c7)cc6)cc5)ccc3n4-c3ccccc3)cc2)cc1]

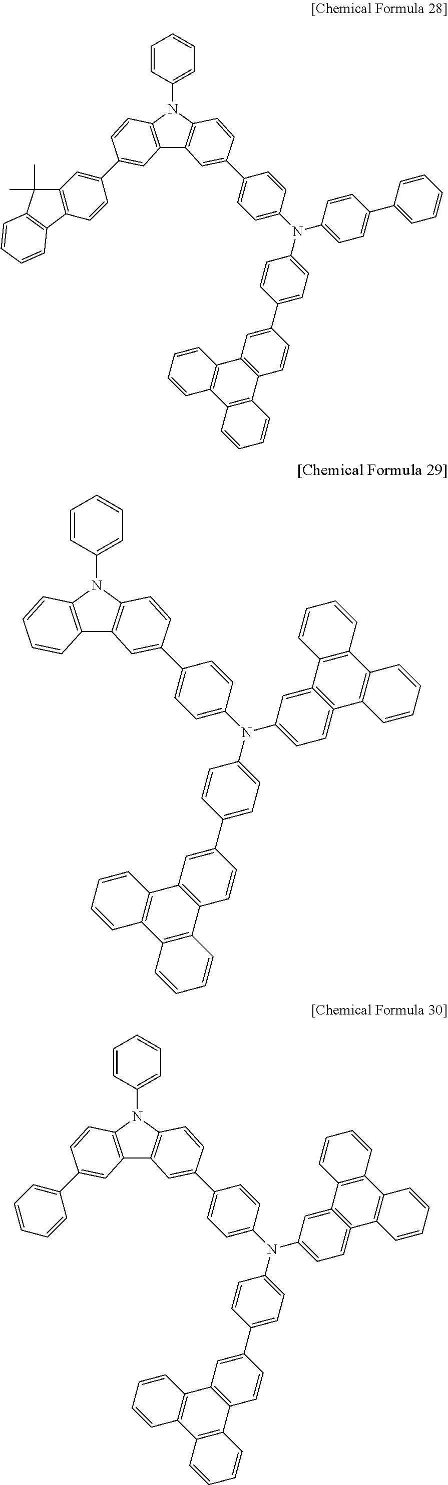

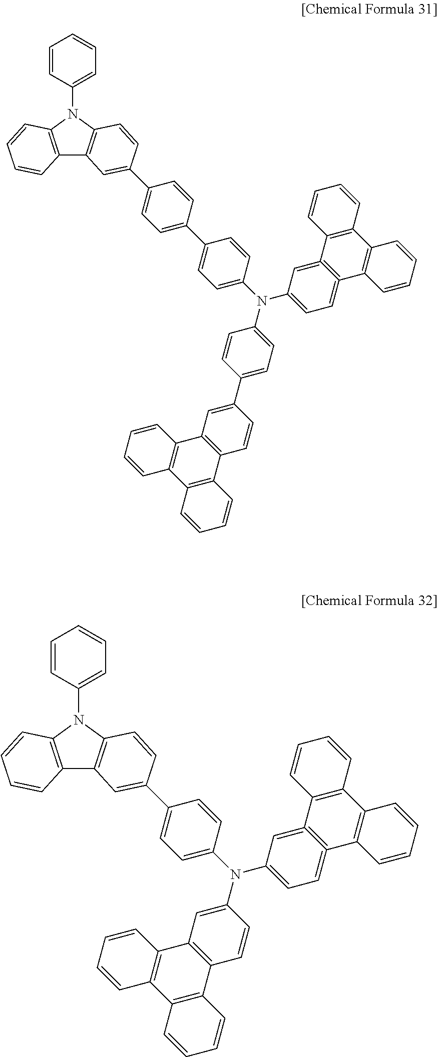

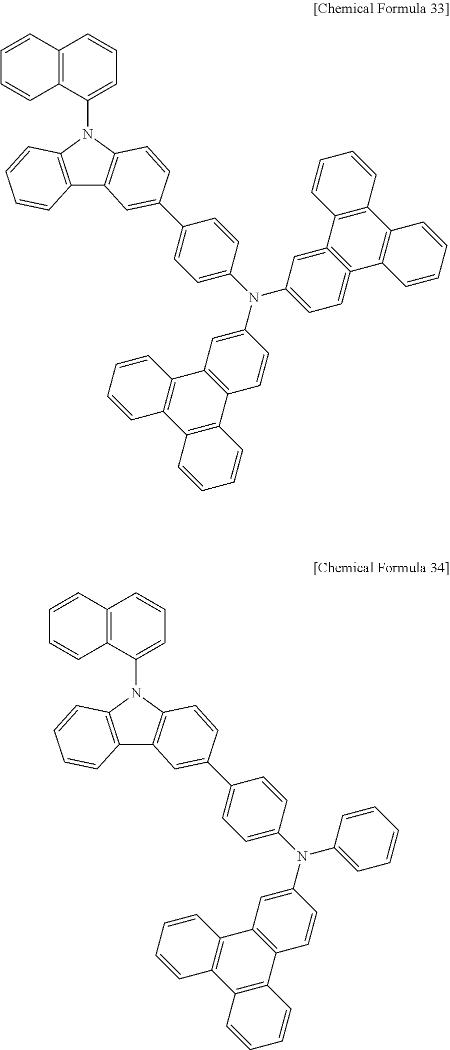

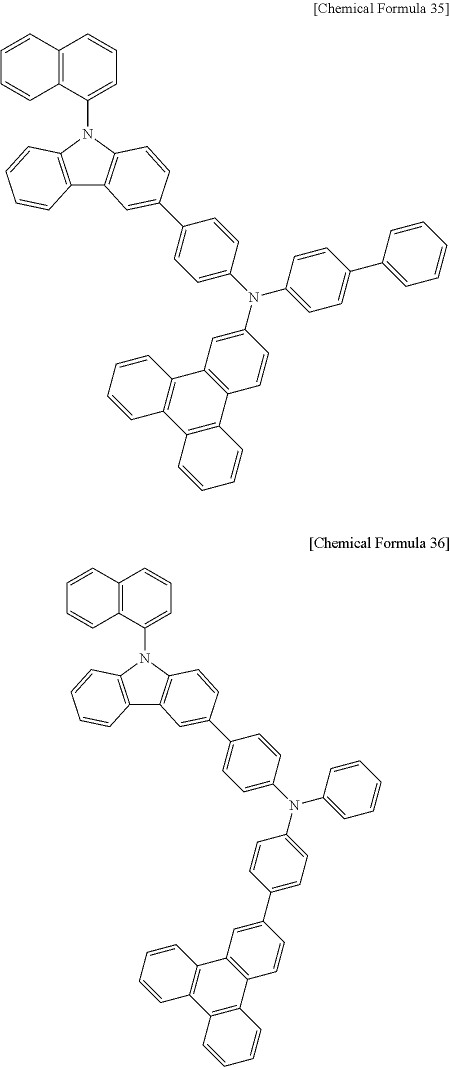 [SMILES: c1ccc(-c2ccc(N(c3ccc(-c4ccc5c(c4)c4ccccc4n5-c4cccc5ccccc45)cc3)c3ccc4c5ccccc5c5ccccc5c4c3)cc2)cc1 ; c1ccc(N(c2ccc(-c3ccc4c5ccccc5c5ccccc5c4c3)cc2)c2ccc(-c3ccc4c(c3)c3ccccc3n4-c3cccc4ccccc34)cc2)cc1]

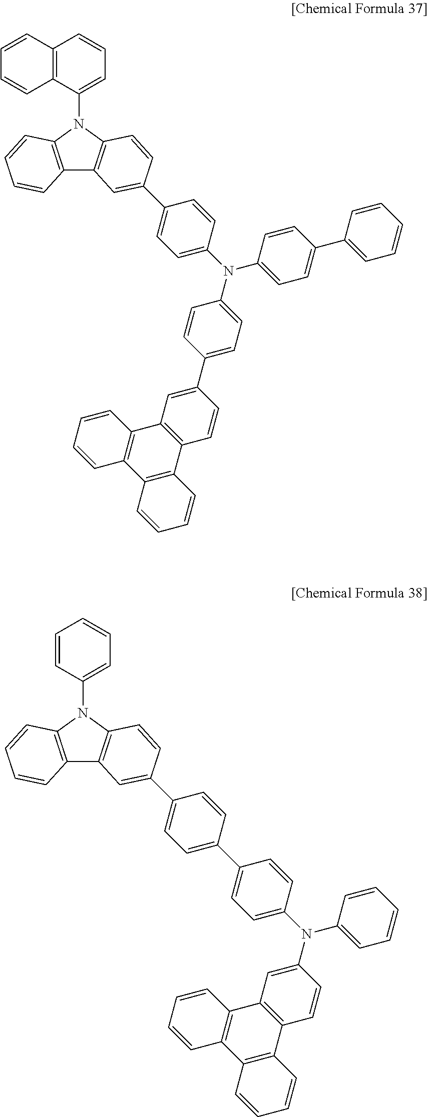 [SMILES: c1ccc(-c2ccc(N(c3ccc(-c4ccc5c6ccccc6c6ccccc6c5c4)cc3)c3ccc(-c4ccc5c(c4)c4ccccc4n5-c4cccc5ccccc45)cc3)cc2)cc1 ; c1ccc(N(c2ccc(-c3ccc(-c4ccc5c(c4)c4ccccc4n5-c4ccccc4)cc3)cc2)c2ccc3c4ccccc4c4ccccc4c3c2)cc1]

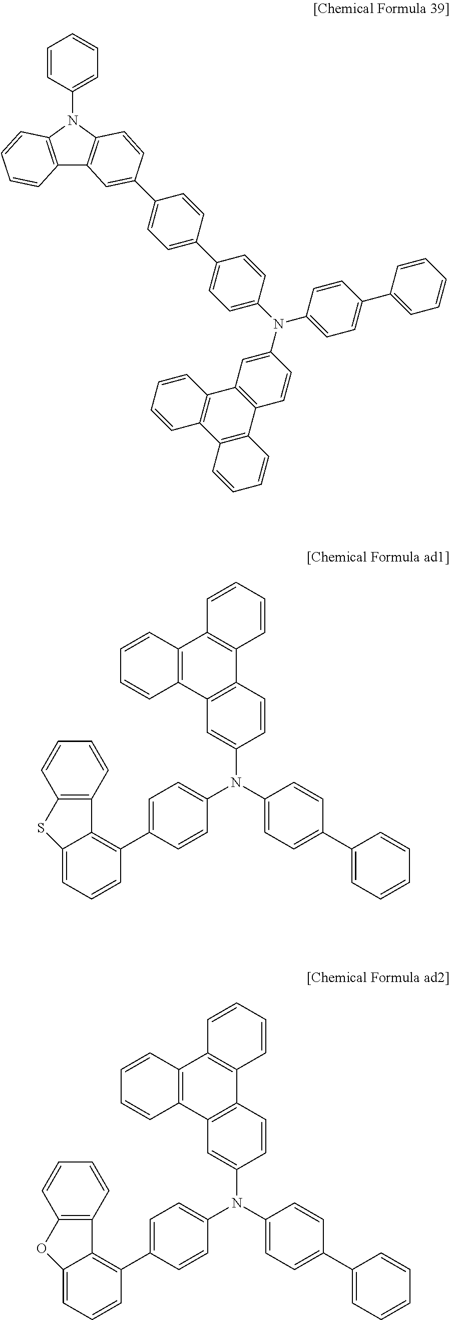 [SMILES: c1ccc(-c2ccc(N(c3ccc(-c4ccc(-c5ccc6c(c5)c5ccccc5n6-c5ccccc5)cc4)cc3)c3ccc4c5ccccc5c5ccccc5c4c3)cc2)cc1 ; c1ccc(-c2ccc(N(c3ccc(-c4cccc5sc6ccccc6c45)cc3)c3ccc4c5ccccc5c5ccccc5c4c3)cc2)cc1 ; c1ccc(-c2ccc(N(c3ccc(-c4cccc5oc6ccccc6c45)cc3)c3ccc4c5ccccc5c5ccccc5c4c3)cc2)cc1]

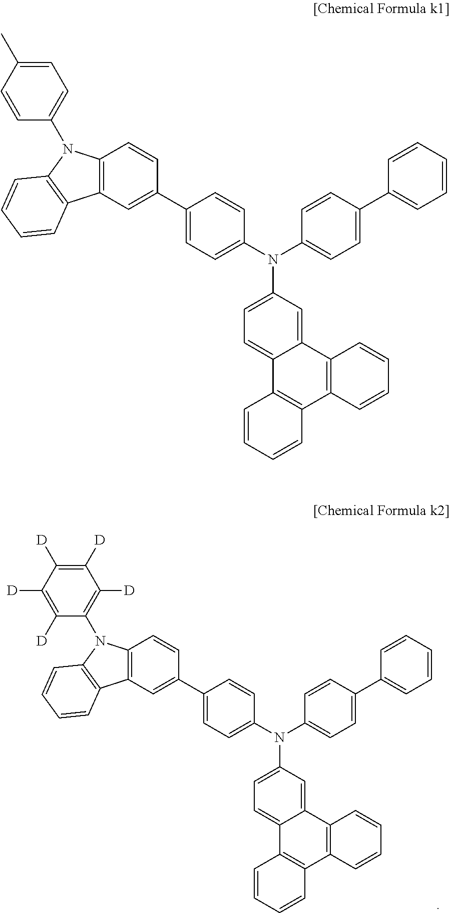 [SMILES: Cc1ccc(-n2c3ccccc3c3cc(-c4ccc(N(c5ccc(-c6ccccc6)cc5)c5ccc6c7ccccc7c7ccccc7c6c5)cc4)ccc32)cc1 ; [2H]c1c([2H])c([2H])c(-n2c3ccccc3c3cc(-c4ccc(N(c5ccc(-c6ccccc6)cc5)c5ccc6c7ccccc7c7ccccc7c6c5)cc4)ccc32)c([2H])c1[2H]]

10. A compound for an organic photoelectric device, the compound being represented by one of the following Chemical Formulae 40 to 106, ad3, ad4, or ad5:

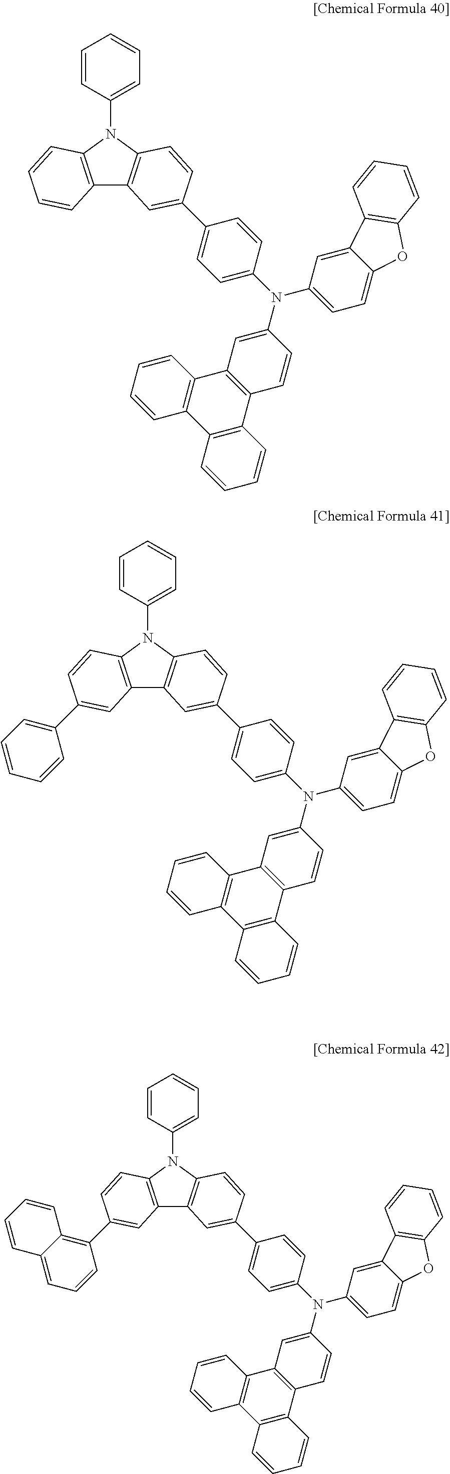 [SMILES: c1ccc(-n2c3ccccc3c3cc(-c4ccc(N(c5ccc6oc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6oc7ccccc7c6c5)c5ccc6c7ccccc7c7ccccc7c6c5)cc4)ccc2n3-c2ccccc2)cc1 ; c1ccc(-n2c3ccc(-c4ccc(N(c5ccc6oc7ccccc7c6c5)c5ccc6c7ccccc7c7ccccc7c6c5)cc4)cc3c3cc(-c4cccc5ccccc45)ccc32)cc1]

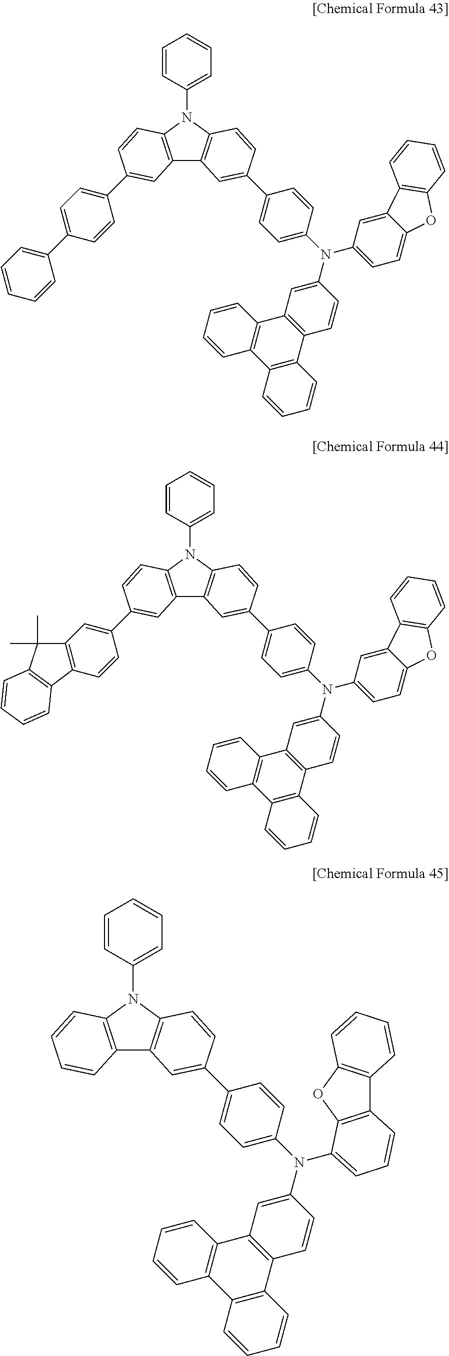 [SMILES: c1ccc(-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc7oc8ccccc8c7c6)c6ccc7c8ccccc8c8ccccc8c7c6)cc5)ccc3n4-c3ccccc3)cc21 ; c1ccc(-n2c3ccccc3c3cc(-c4ccc(N(c5ccc6c7ccccc7c7ccccc7c6c5)c5cccc6c5oc5ccccc56)cc4)ccc32)cc1]

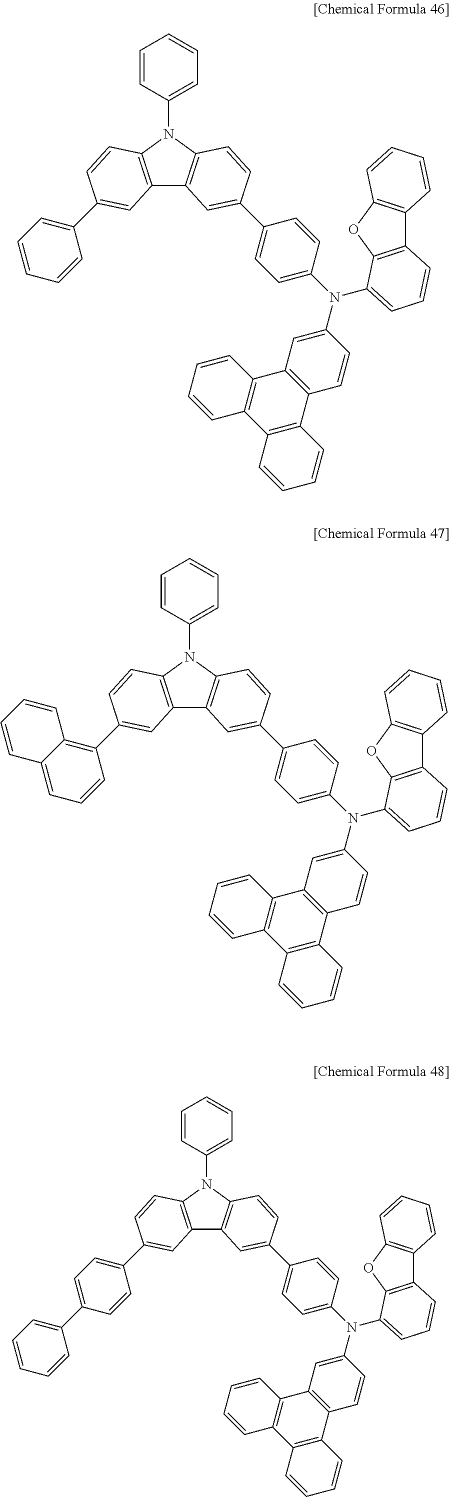 [SMILES: c1ccc(-c2ccc3c(c2)c2cc(-c4ccc(N(c5ccc6c7ccccc7c7ccccc7c6c5)c5cccc6c5oc5ccccc56)cc4)ccc2n3-c2ccccc2)cc1 ; c1ccc(-n2c3ccc(-c4ccc(N(c5ccc6c7ccccc7c7ccccc7c6c5)c5cccc6c5oc5ccccc56)cc4)cc3c3cc(-c4cccc5ccccc45)ccc32)cc1 ; c1ccc(-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccccc3)cc2)cc1]

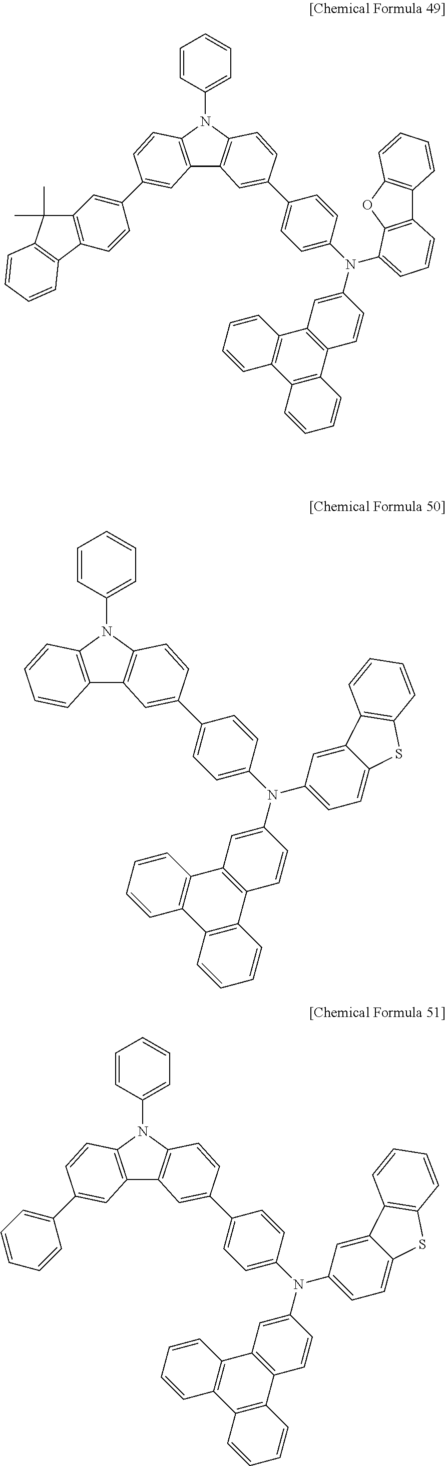 [SMILES: CC1(C)c2ccccc2-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccccc3)cc21 ; c1ccc(-n2c3ccccc3c3cc(-c4ccc(N(c5ccc6sc7ccccc7c6c5)c5ccc6c7ccccc7c7ccccc7c6c5)cc4)ccc32)cc1 ; c1ccc(-c2ccc3c(c2)c2cc(-c4ccc(N(c5ccc6sc7ccccc7c6c5)c5ccc6c7ccccc7c7ccccc7c6c5)cc4)ccc2n3-c2ccccc2)cc1]

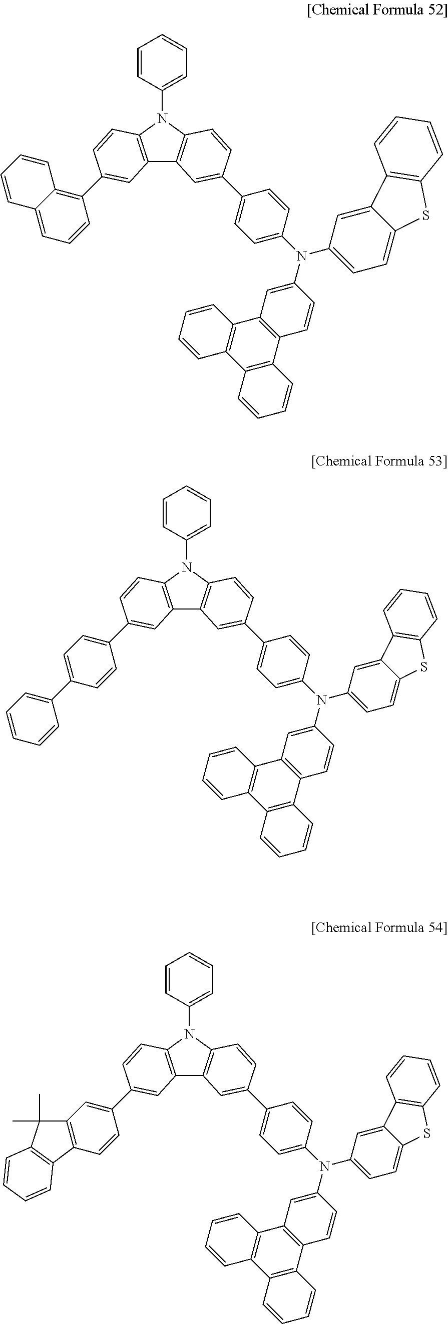 [SMILES: c1ccc(-n2c3ccc(-c4ccc(N(c5ccc6sc7ccccc7c6c5)c5ccc6c7ccccc7c7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccccc3)cc21]

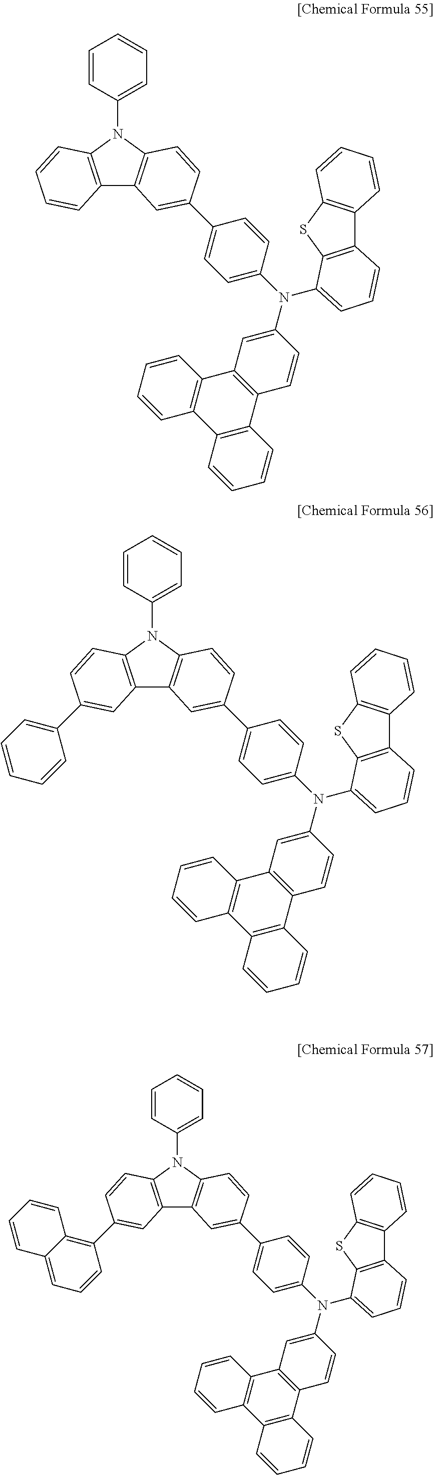 [SMILES: c1ccc(-n2c3ccccc3c3cc(-c4ccc(N(c5ccc6c7ccccc7c7ccccc7c6c5)c5cccc6c5sc5ccccc56)cc4)ccc32)cc1 ; c1ccc(-c2ccc3c(c2)c2cc(-c4ccc(N(c5ccc6c7ccccc7c7ccccc7c6c5)c5cccc6c5sc5ccccc56)cc4)ccc2n3-c2ccccc2)cc1 ; c1ccc(-n2c3ccc(-c4ccc(N(c5ccc6c7ccccc7c7ccccc7c6c5)c5cccc6c5sc5ccccc56)cc4)cc3c3cc(-c4cccc5ccccc45)ccc32)cc1]

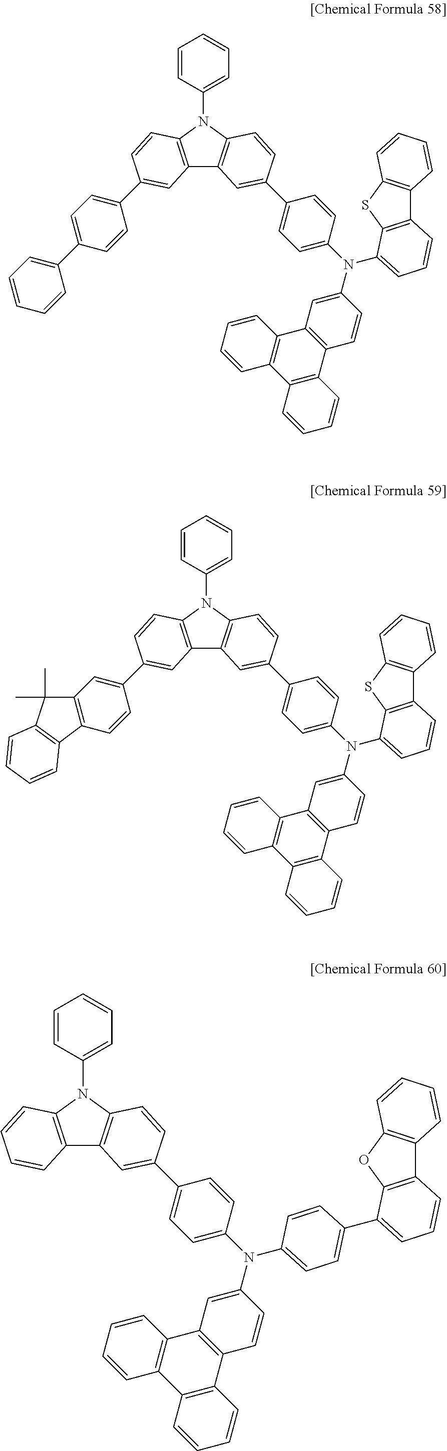 [SMILES: c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccccc3)cc21 ; c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1]

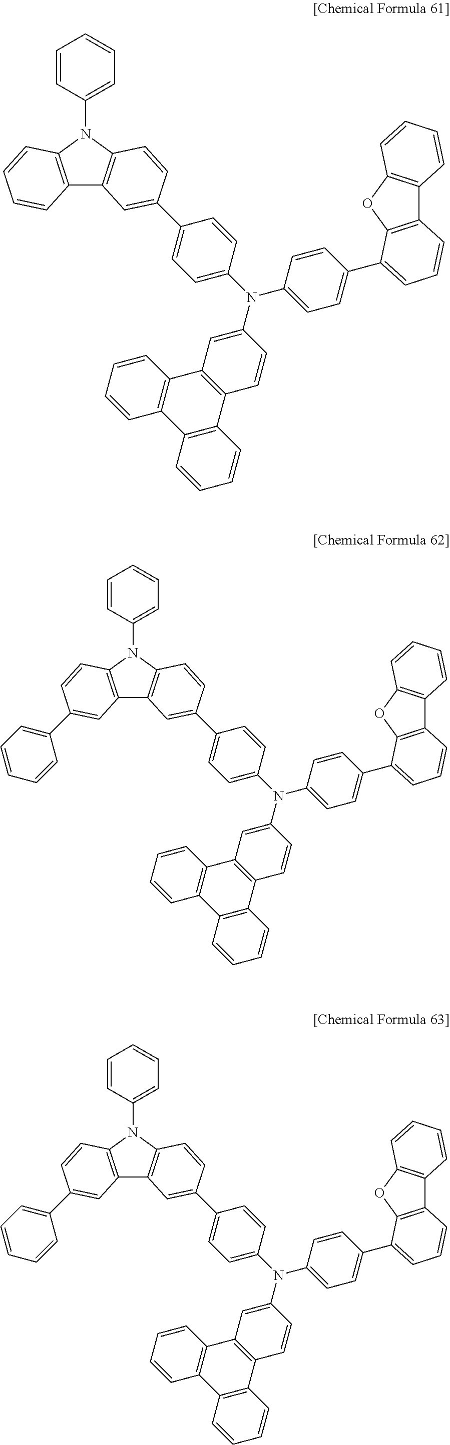 [SMILES: c1ccc(-n2c3ccccc3c3cc(-c4ccc(N(c5ccc(-c6cccc7c6oc6ccccc67)cc5)c5ccc6c7ccccc7c7ccccc7c6c5)cc4)ccc32)cc1 ; c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1 ; c1ccc(-c2ccc3c(c2)c2cc(-c4ccc(N(c5ccc(-c6cccc7c6oc6ccccc67)cc5)c5ccc6c7ccccc7c7ccccc7c6c5)cc4)ccc2n3-c2ccccc2)cc1]

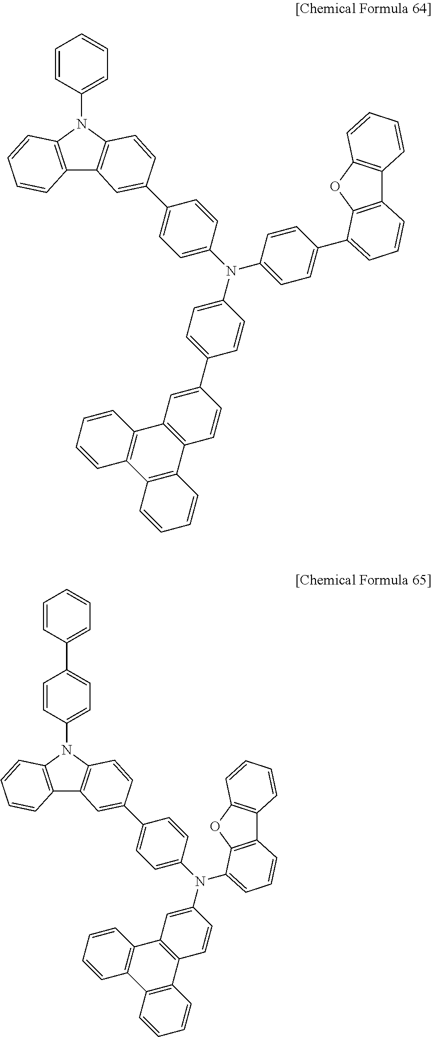 [SMILES: c1ccc(-n2c3ccccc3c3cc(-c4ccc(N(c5ccc(-c6ccc7c8ccccc8c8ccccc8c7c6)cc5)c5ccc(-c6cccc7c6oc6ccccc67)cc5)cc4)ccc32)cc1 ; c1ccc(-c2ccc(-n3c4ccccc4c4cc(-c5ccc(N(c6ccc7c8ccccc8c8ccccc8c7c6)c6cccc7c6oc6ccccc67)cc5)ccc43)cc2)cc1]

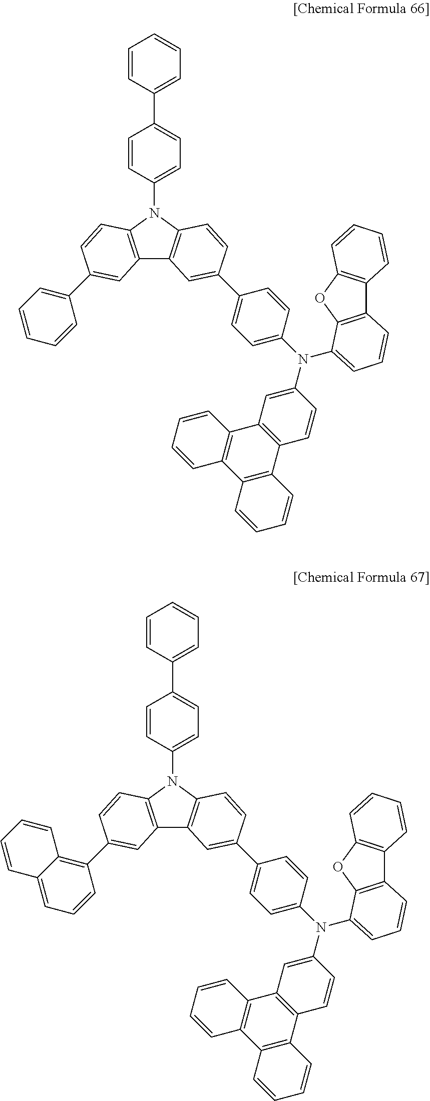 [SMILES: c1ccc(-c2ccc(-n3c4ccc(-c5ccccc5)cc4c4cc(-c5ccc(N(c6ccc7c8ccccc8c8ccccc8c7c6)c6cccc7c6oc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6oc6ccccc67)cc5)cc4c4cc(-c5cccc6ccccc56)ccc43)cc2)cc1]

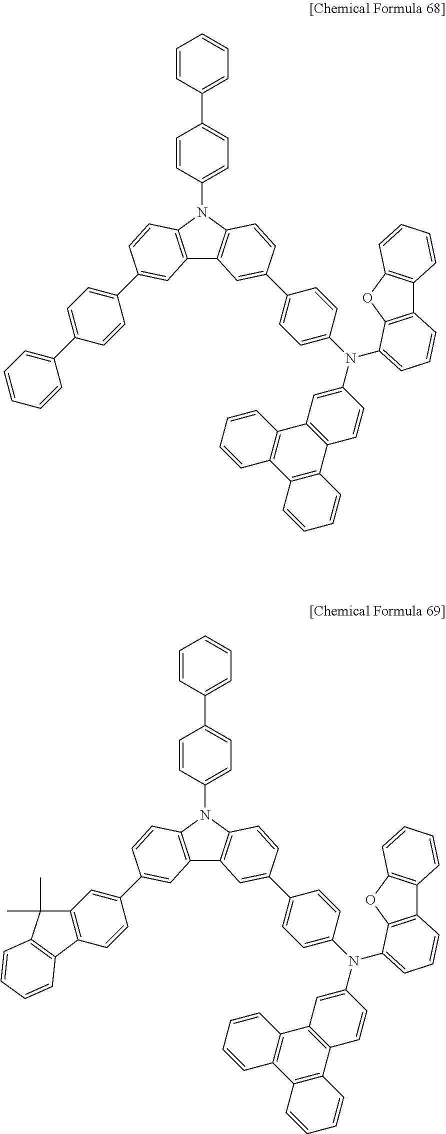 [SMILES: c1ccc(-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6oc6ccccc67)cc5)ccc3n4-c3ccc(-c4ccccc4)cc3)cc21]

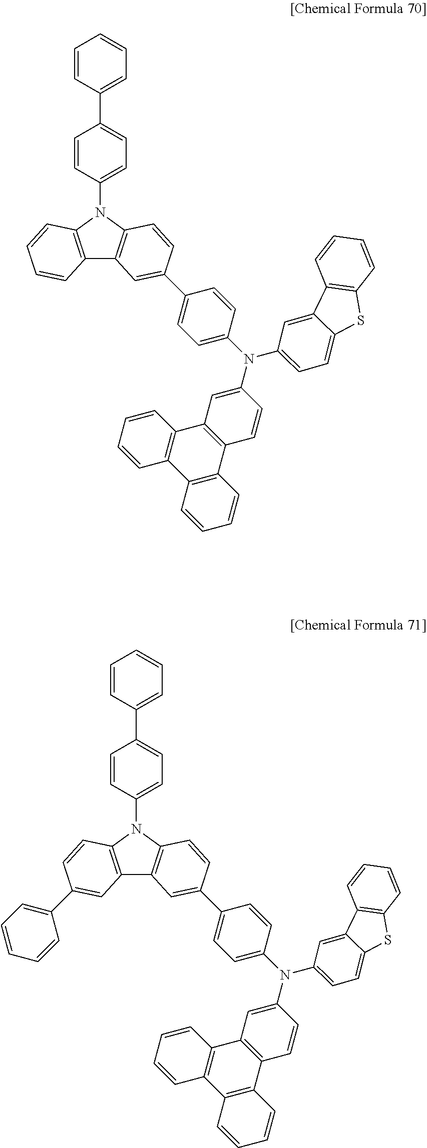 [SMILES: c1ccc(-c2ccc(-n3c4ccccc4c4cc(-c5ccc(N(c6ccc7sc8ccccc8c7c6)c6ccc7c8ccccc8c8ccccc8c7c6)cc5)ccc43)cc2)cc1 ; c1ccc(-c2ccc(-n3c4ccc(-c5ccccc5)cc4c4cc(-c5ccc(N(c6ccc7sc8ccccc8c7c6)c6ccc7c8ccccc8c8ccccc8c7c6)cc5)ccc43)cc2)cc1]

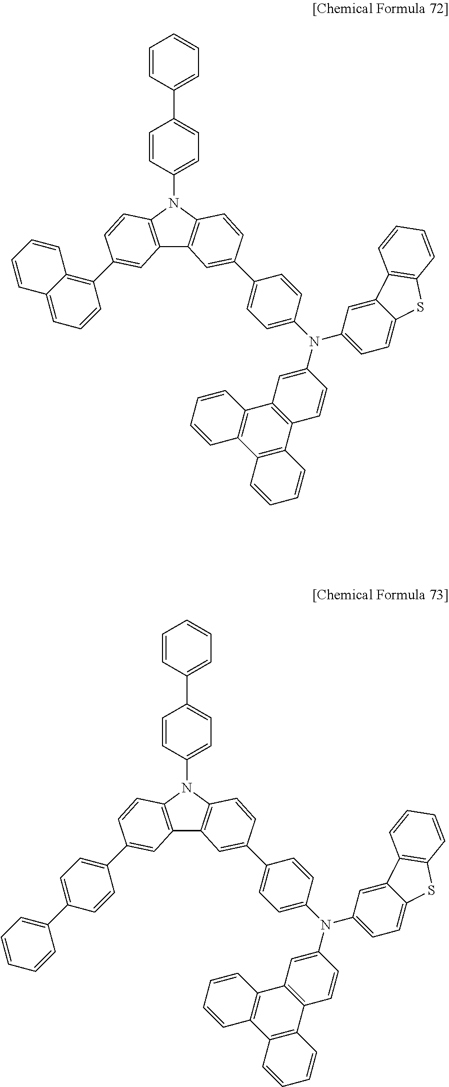 [SMILES: c1ccc(-c2ccc(-n3c4ccc(-c5ccc(N(c6ccc7sc8ccccc8c7c6)c6ccc7c8ccccc8c8ccccc8c7c6)cc5)cc4c4cc(-c5cccc6ccccc56)ccc43)cc2)cc1 ; c1ccc(-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc2)cc1]

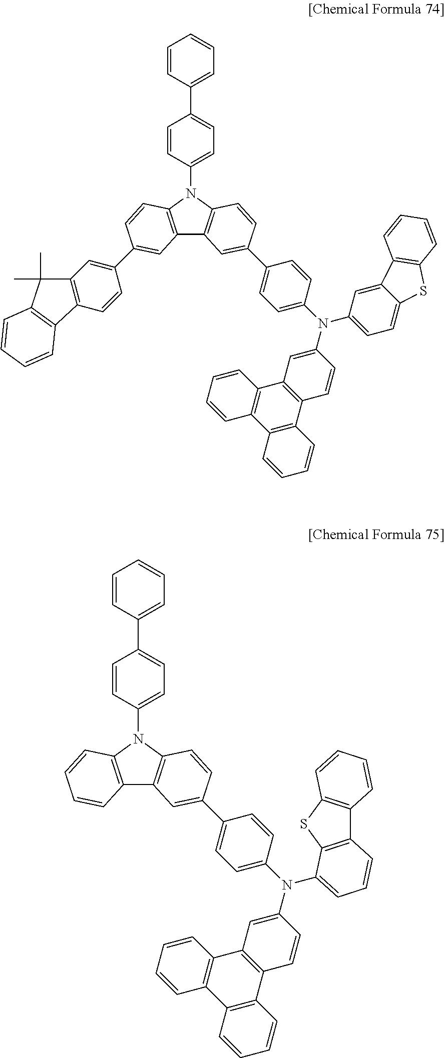 [SMILES: CC1(C)c2ccccc2-c2ccc(-c3ccc4c(c3)c3cc(-c5ccc(N(c6ccc7sc8ccccc8c7c6)c6ccc7c8ccccc8c8ccccc8c7c6)cc5)ccc3n4-c3ccc(-c4ccccc4)cc3)cc21 ; c1ccc(-c2ccc(-n3c4ccccc4c4cc(-c5ccc(N(c6ccc7c8ccccc8c8ccccc8c7c6)c6cccc7c6sc6ccccc67)cc5)ccc43)cc2)cc1]

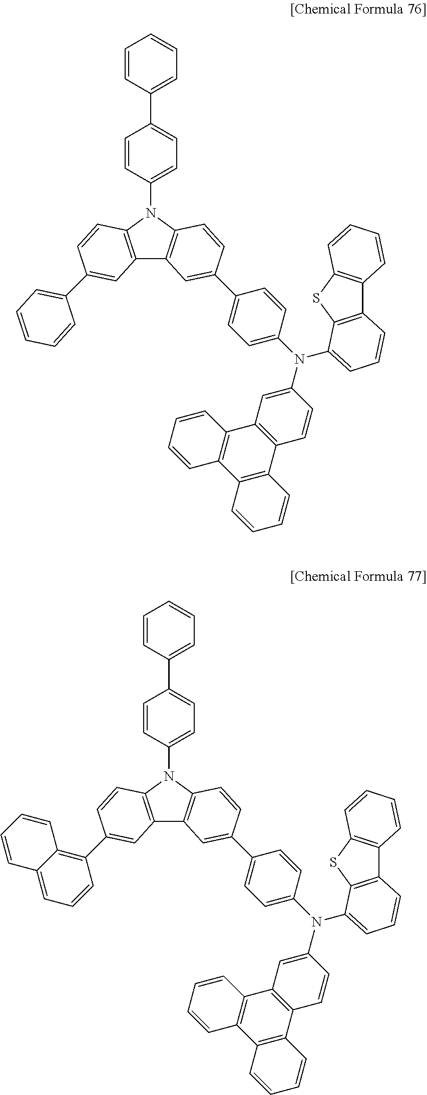 [SMILES: c1ccc(-c2ccc(-n3c4ccc(-c5ccccc5)cc4c4cc(-c5ccc(N(c6ccc7c8ccccc8c8ccccc8c7c6)c6cccc7c6sc6ccccc67)cc5)ccc43)cc2)cc1 ; c1ccc(-c2ccc(-n3c4ccc(-c5ccc(N(c6ccc7c8ccccc8c8ccccc8c7c6)c6cccc7c6sc6ccccc67)cc5)cc4c4cc(-c5cccc6ccccc56)ccc43)cc2)cc1]

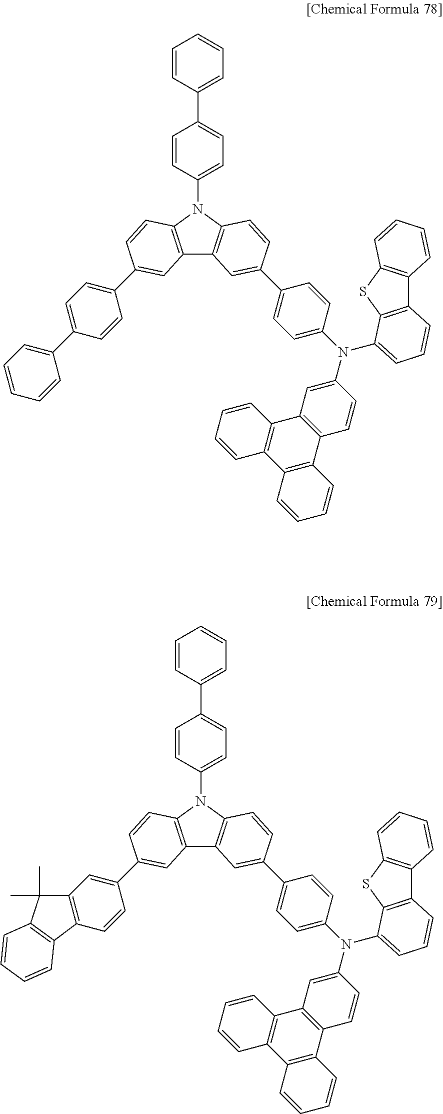 [SMILES: c1ccc(-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc2)cc1 ; CC1(C)c2ccccc2-c2ccc(-c3ccc4c(c3)c3cc(-c5ccc(N(c6ccc7c8ccccc8c8ccccc8c7c6)c6cccc7c6sc6ccccc67)cc5)ccc3n4-c3ccc(-c4ccccc4)cc3)cc21]

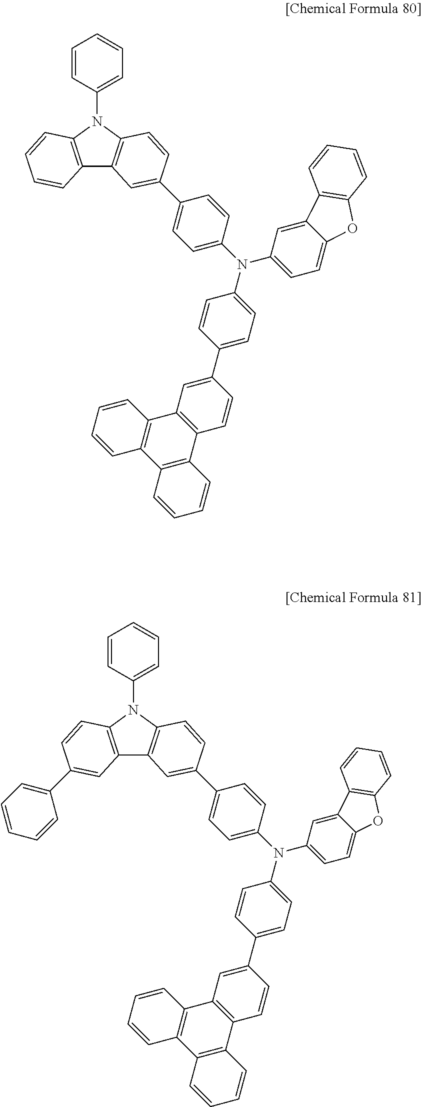 [SMILES: c1ccc(-n2c3ccccc3c3cc(-c4ccc(N(c5ccc(-c6ccc7c8ccccc8c8ccccc8c7c6)cc5)c5ccc6oc7ccccc7c6c5)cc4)ccc32)cc1 ; c1ccc(-c2ccc3c(c2)c2cc(-c4ccc(N(c5ccc(-c6ccc7c8ccccc8c8ccccc8c7c6)cc5)c5ccc6oc7ccccc7c6c5)cc4)ccc2n3-c2ccccc2)cc1]

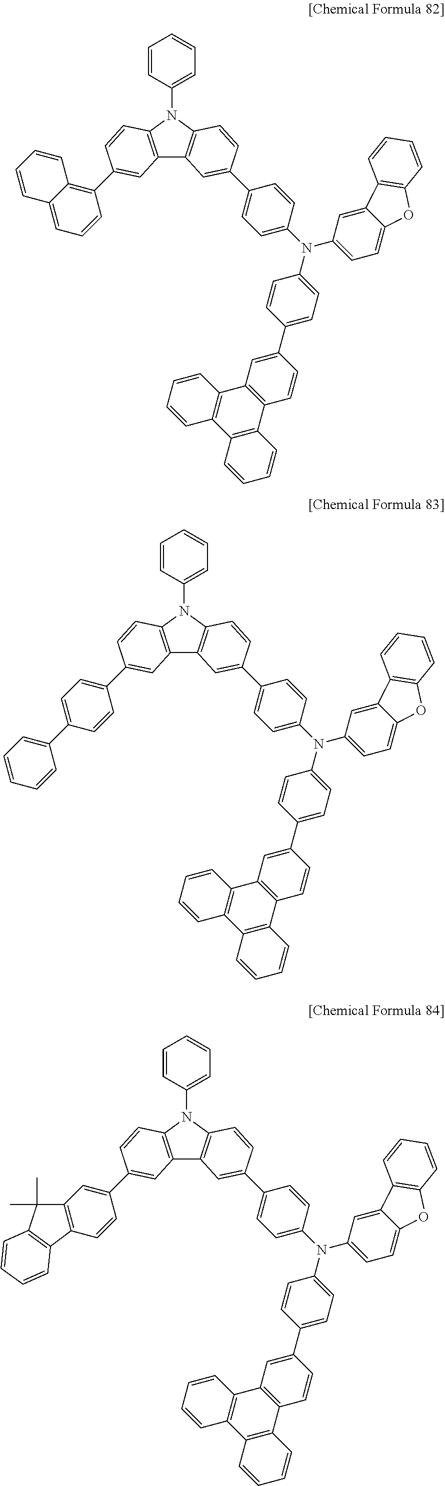 [SMILES: c1ccc(-n2c3ccc(-c4ccc(N(c5ccc(-c6ccc7c8ccccc8c8ccccc8c7c6)cc5)c5ccc6oc7ccccc7c6c5)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6ccc7oc8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6ccc7oc8ccccc8c7c6)cc5)ccc3n4-c3ccccc3)cc21]

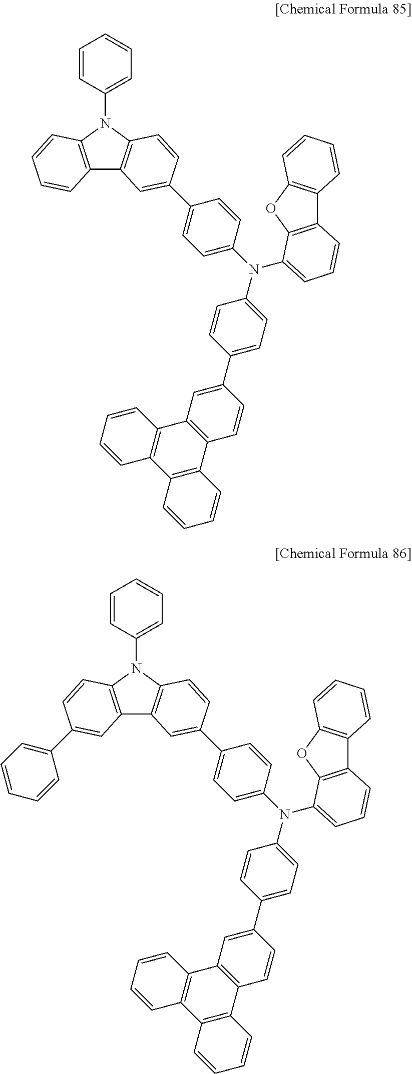 [SMILES: c1ccc(-n2c3ccccc3c3cc(-c4ccc(N(c5ccc(-c6ccc7c8ccccc8c8ccccc8c7c6)cc5)c5cccc6c5oc5ccccc56)cc4)ccc32)cc1 ; c1ccc(-c2ccc3c(c2)c2cc(-c4ccc(N(c5ccc(-c6ccc7c8ccccc8c8ccccc8c7c6)cc5)c5cccc6c5oc5ccccc56)cc4)ccc2n3-c2ccccc2)cc1]

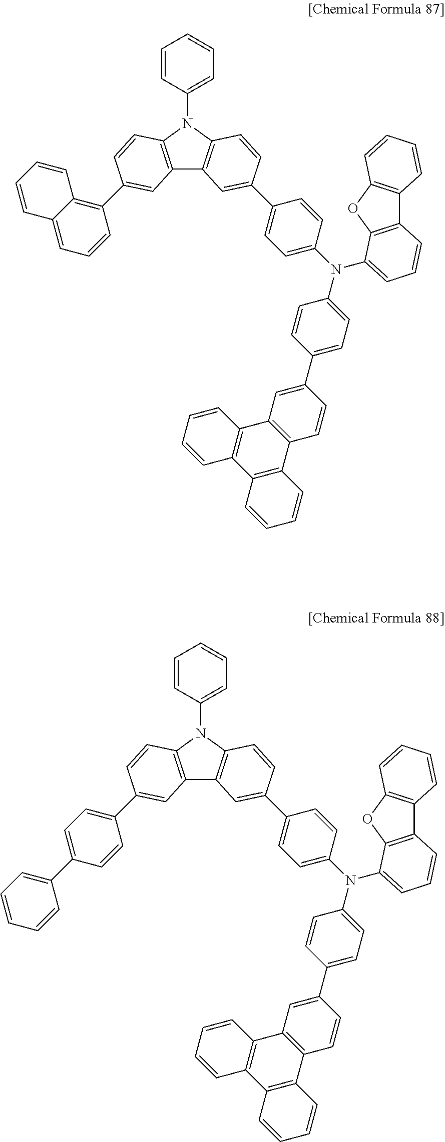 [SMILES: c1ccc(-n2c3ccc(-c4ccc(N(c5ccc(-c6ccc7c8ccccc8c8ccccc8c7c6)cc5)c5cccc6c5oc5ccccc56)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc2)cc1]

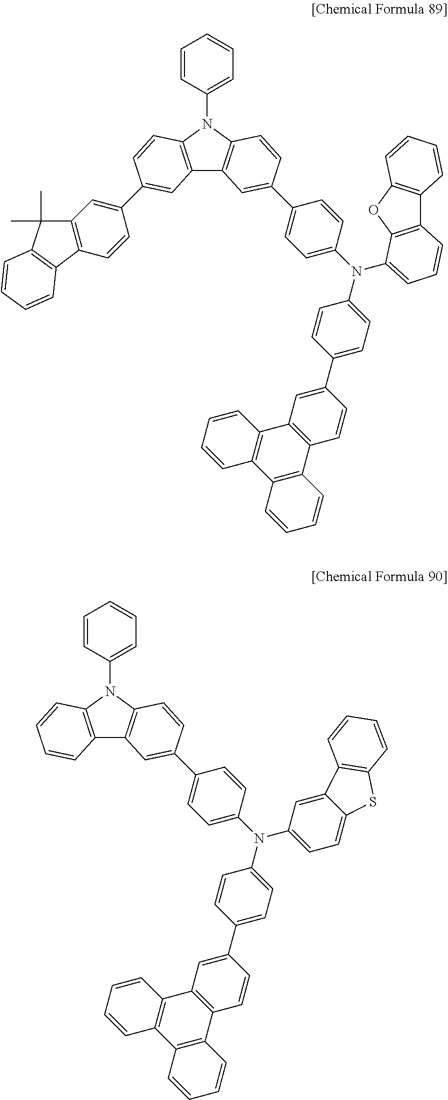 [SMILES: CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6oc6ccccc67)cc5)ccc3n4-c3ccccc3)cc21 ; c1ccc(-n2c3ccccc3c3cc(-c4ccc(N(c5ccc(-c6ccc7c8ccccc8c8ccccc8c7c6)cc5)c5ccc6sc7ccccc7c6c5)cc4)ccc32)cc1]

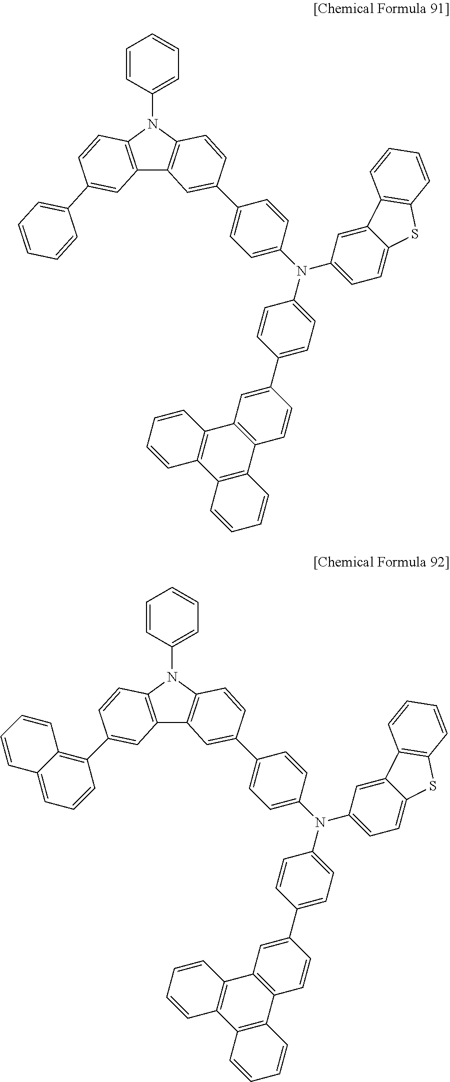 [SMILES: c1ccc(-c2ccc3c(c2)c2cc(-c4ccc(N(c5ccc(-c6ccc7c8ccccc8c8ccccc8c7c6)cc5)c5ccc6sc7ccccc7c6c5)cc4)ccc2n3-c2ccccc2)cc1 ; c1ccc(-n2c3ccc(-c4ccc(N(c5ccc(-c6ccc7c8ccccc8c8ccccc8c7c6)cc5)c5ccc6sc7ccccc7c6c5)cc4)cc3c3cc(-c4cccc5ccccc45)ccc32)cc1]

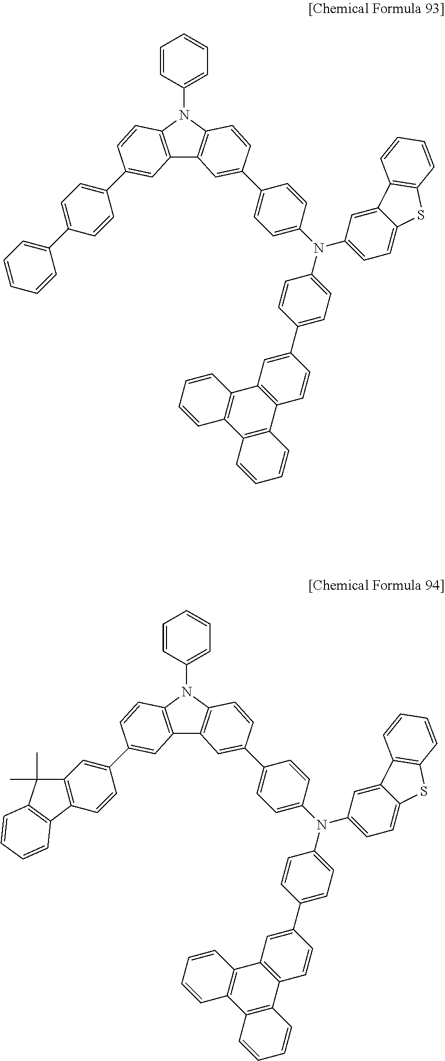 [SMILES: c1ccc(-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc2)cc1 ; CC1(C)c2ccccc2-c2ccc(-c3ccc4c(c3)c3cc(-c5ccc(N(c6ccc(-c7ccc8c9ccccc9c9ccccc9c8c7)cc6)c6ccc7sc8ccccc8c7c6)cc5)ccc3n4-c3ccccc3)cc21]

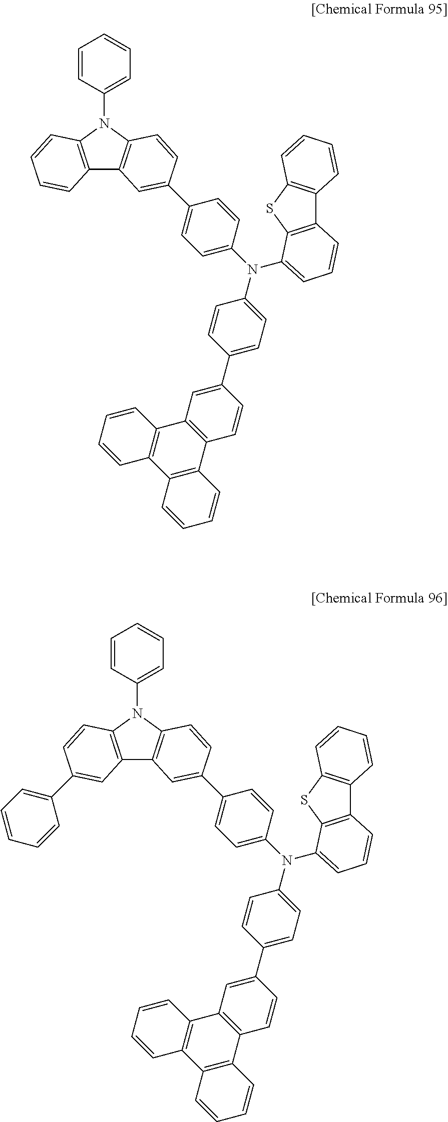 [SMILES: c1ccc(-n2c3ccccc3c3cc(-c4ccc(N(c5ccc(-c6ccc7c8ccccc8c8ccccc8c7c6)cc5)c5cccc6c5sc5ccccc56)cc4)ccc32)cc1 ; c1ccc(-c2ccc3c(c2)c2cc(-c4ccc(N(c5ccc(-c6ccc7c8ccccc8c8ccccc8c7c6)cc5)c5cccc6c5sc5ccccc56)cc4)ccc2n3-c2ccccc2)cc1]

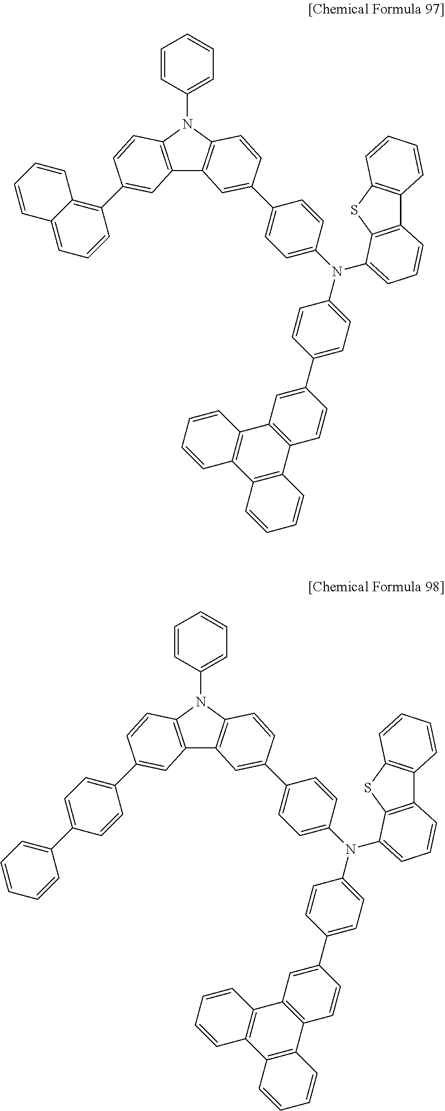 [SMILES: c1ccc(-n2c3ccc(-c4ccc(N(c5ccc(-c6ccc7c8ccccc8c8ccccc8c7c6)cc5)c5cccc6c5sc5ccccc56)cc4)cc3c3cc(-c4cccc5ccccc45)ccc32)cc1 ; c1ccc(-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc2)cc1]

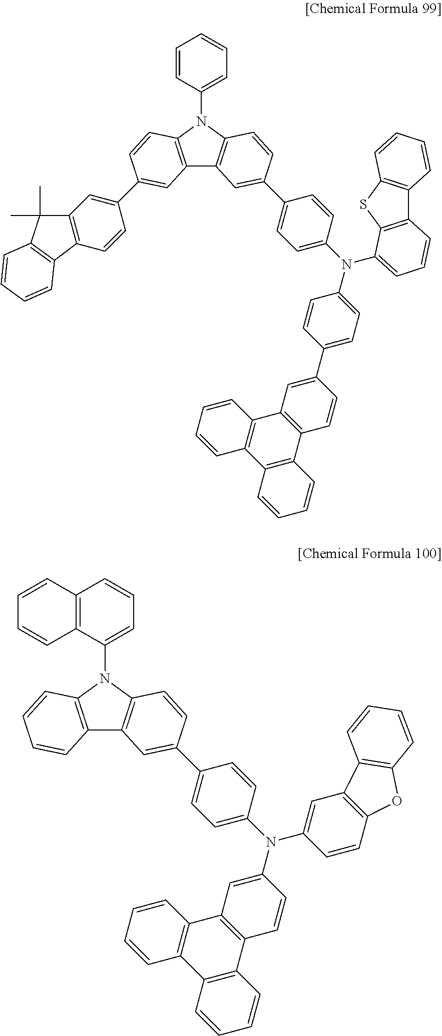 [SMILES: CC1(C)c2ccccc2-c2ccc(-c3ccc4c(c3)c3cc(-c5ccc(N(c6ccc(-c7ccc8c9ccccc9c9ccccc9c8c7)cc6)c6cccc7c6sc6ccccc67)cc5)ccc3n4-c3ccccc3)cc21 ; c1ccc2c(-n3c4ccccc4c4cc(-c5ccc(N(c6ccc7oc8ccccc8c7c6)c6ccc7c8ccccc8c8ccccc8c7c6)cc5)ccc43)cccc2c1]

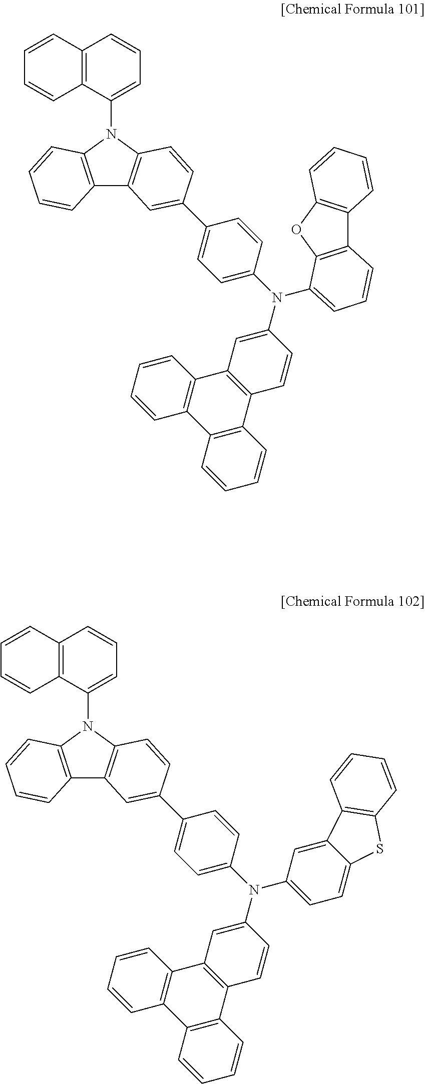 [SMILES: c1ccc2c(-n3c4ccccc4c4cc(-c5ccc(N(c6ccc7c8ccccc8c8ccccc8c7c6)c6cccc7c6oc6ccccc67)cc5)ccc43)cccc2c1 ; c1ccc2c(-n3c4ccccc4c4cc(-c5ccc(N(c6ccc7sc8ccccc8c7c6)c6ccc7c8ccccc8c8ccccc8c7c6)cc5)ccc43)cccc2c1]

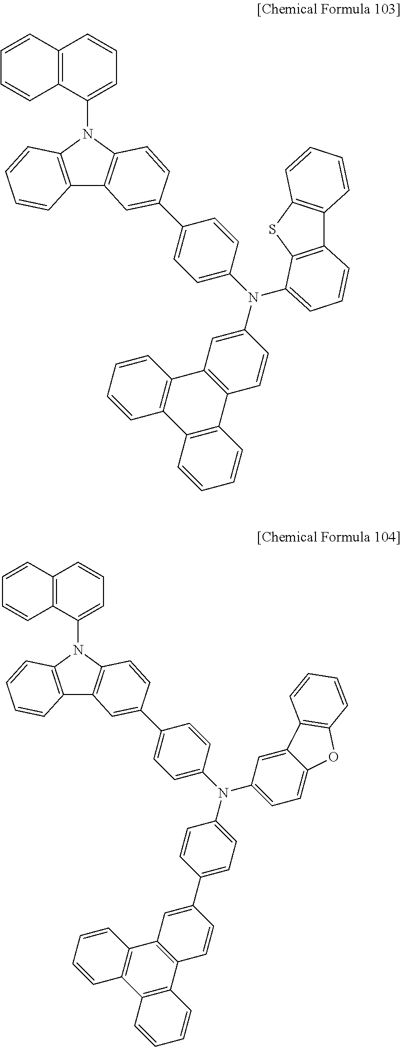 [SMILES: c1ccc2c(-n3c4ccccc4c4cc(-c5ccc(N(c6ccc7c8ccccc8c8ccccc8c7c6)c6cccc7c6sc6ccccc67)cc5)ccc43)cccc2c1 ; c1ccc2c(-n3c4ccccc4c4cc(-c5ccc(N(c6ccc(-c7ccc8c9ccccc9c9ccccc9c8c7)cc6)c6ccc7oc8ccccc8c7c6)cc5)ccc43)cccc2c1]

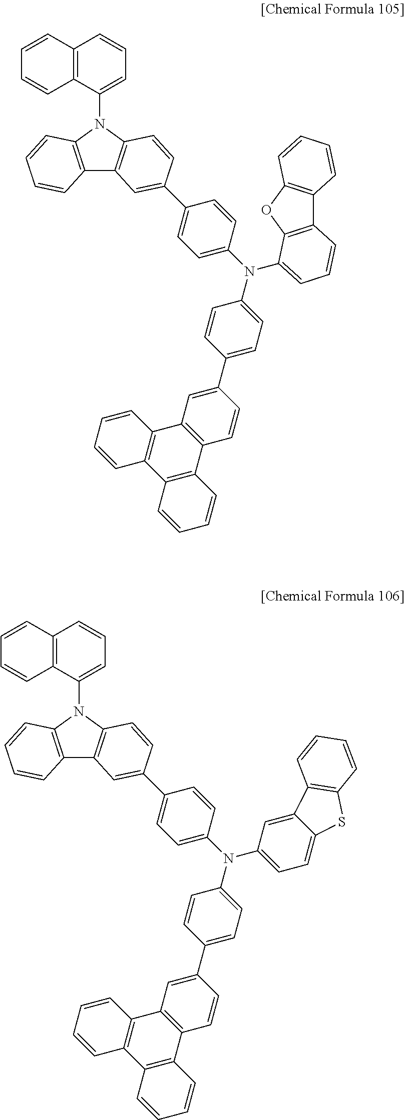 [SMILES: c1ccc2c(-n3c4ccccc4c4cc(-c5ccc(N(c6ccc(-c7ccc8c9ccccc9c9ccccc9c8c7)cc6)c6cccc7c6oc6ccccc67)cc5)ccc43)cccc2c1 ; c1ccc2c(-n3c4ccccc4c4cc(-c5ccc(N(c6ccc(-c7ccc8c9ccccc9c9ccccc9c8c7)cc6)c6ccc7sc8ccccc8c7c6)cc5)ccc43)cccc2c1]

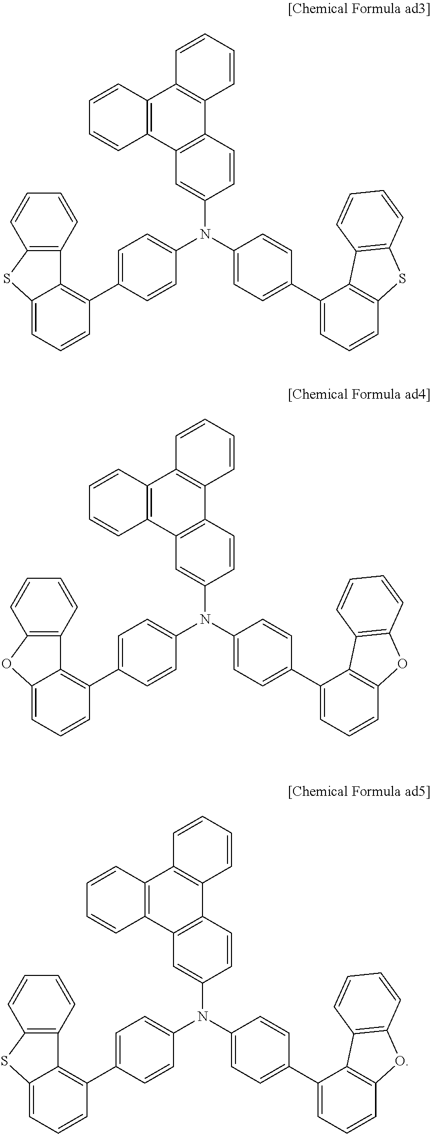 [SMILES: c1ccc2c(c1)sc1cccc(-c3ccc(N(c4ccc(-c5cccc6sc7ccccc7c56)cc4)c4ccc5c6ccccc6c6ccccc6c5c4)cc3)c12 ; c1ccc2c(c1)oc1cccc(-c3ccc(N(c4ccc(-c5cccc6oc7ccccc7c56)cc4)c4ccc5c6ccccc6c6ccccc6c5c4)cc3)c12 ; c1ccc2c(c1)oc1cccc(-c3ccc(N(c4ccc(-c5cccc6sc7ccccc7c56)cc4)c4ccc5c6ccccc6c6ccccc6c5c4)cc3)c12]

11. A compound for an organic photoelectric device, the compound being represented by one of the following Chemical Formulae 107 to 333:

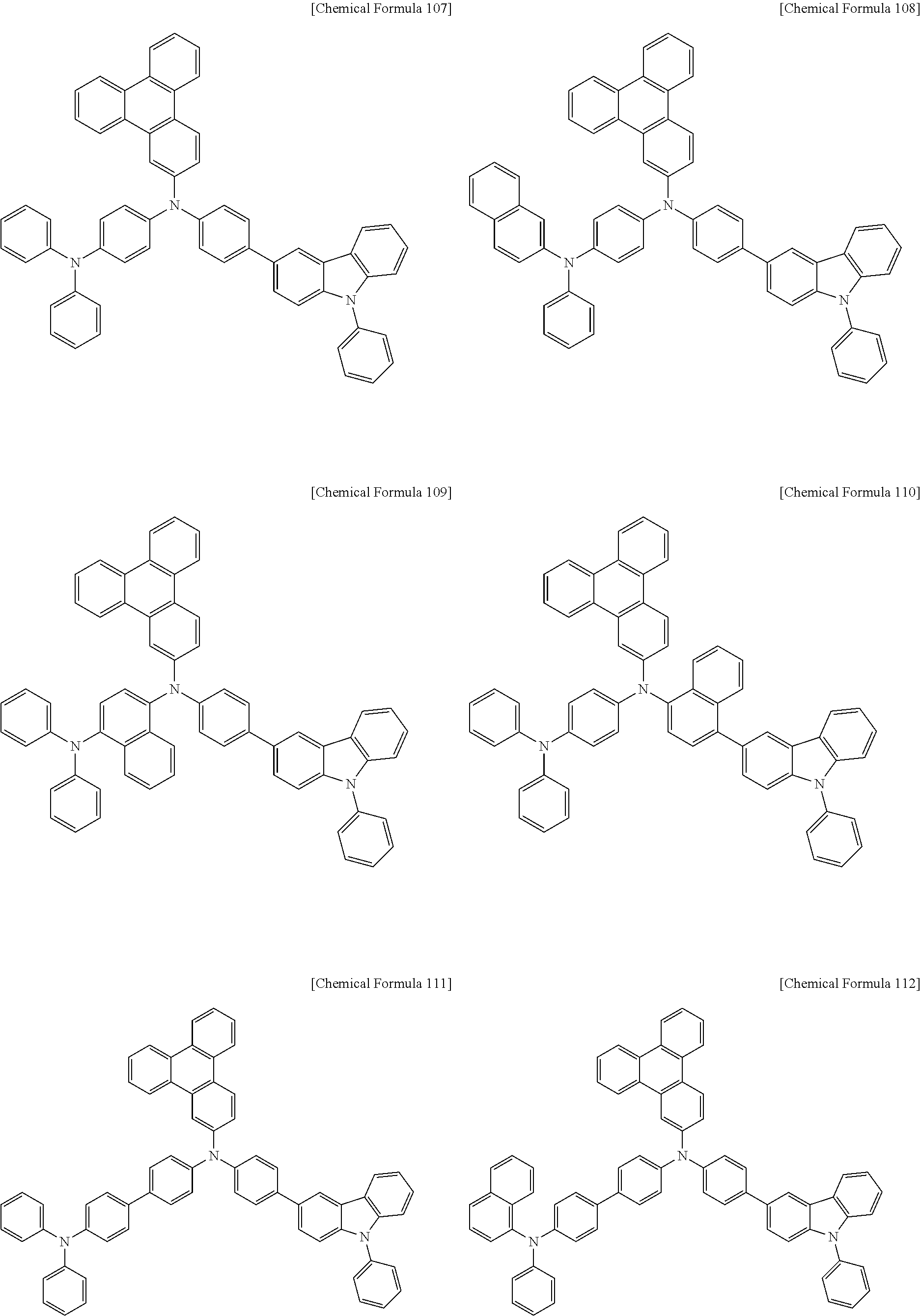

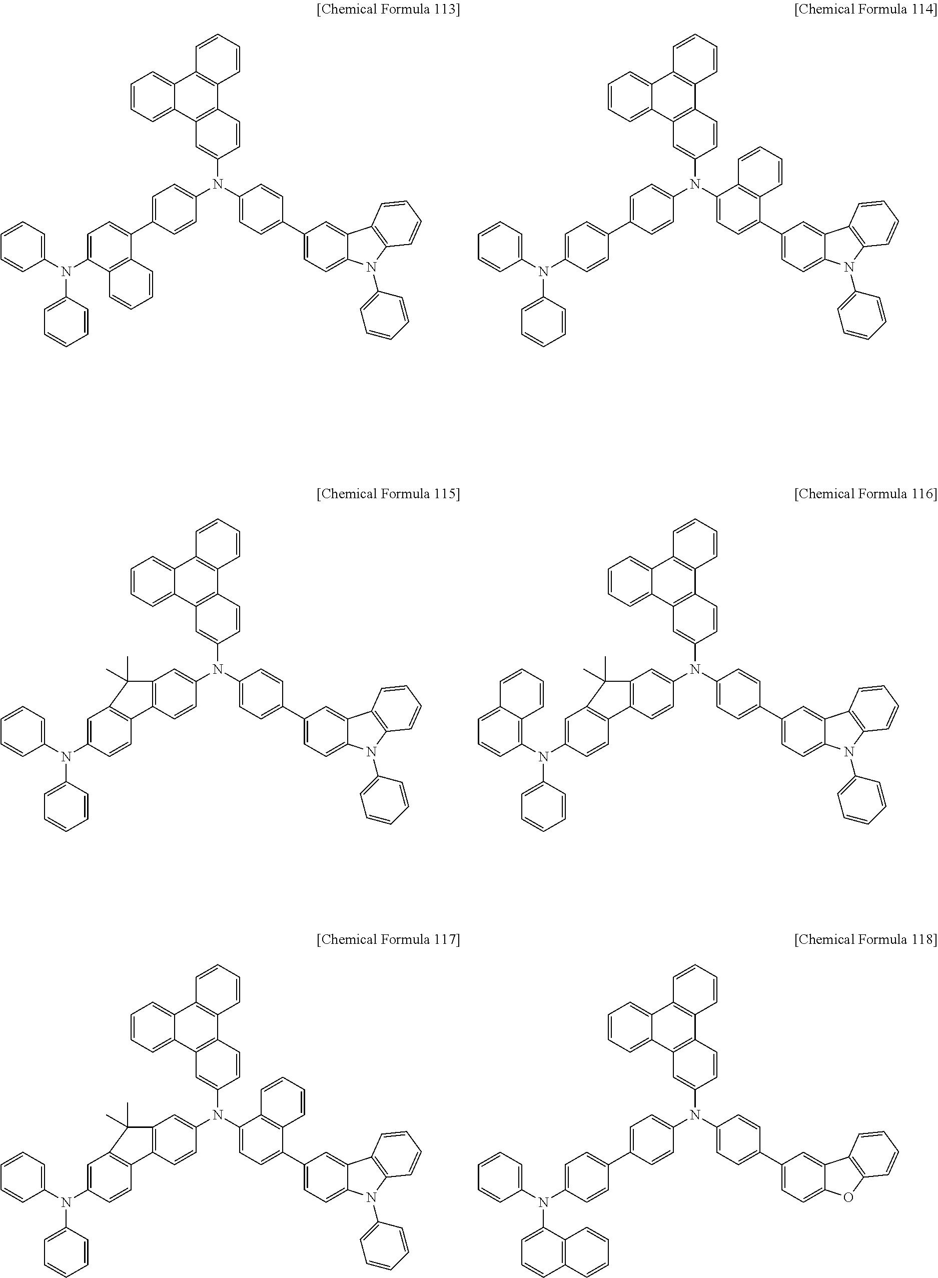

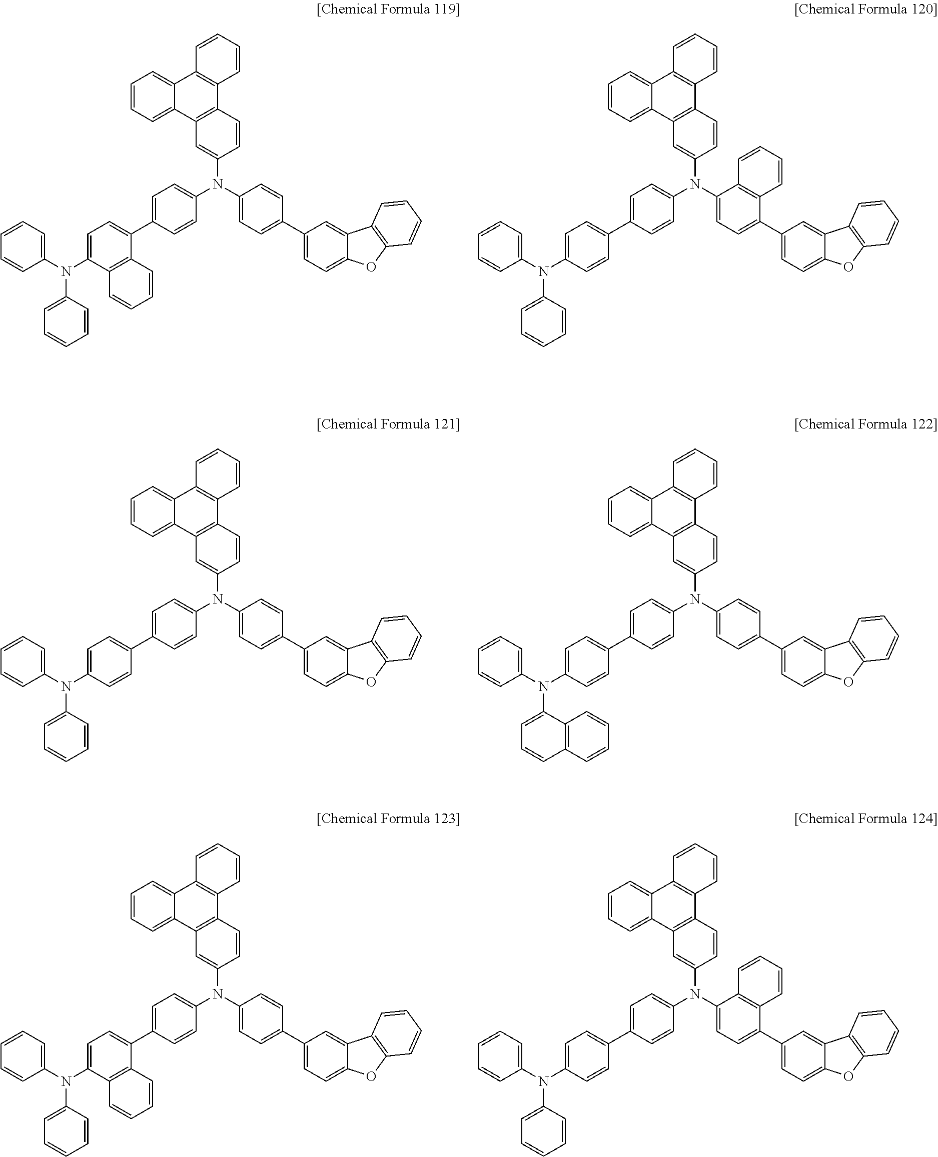

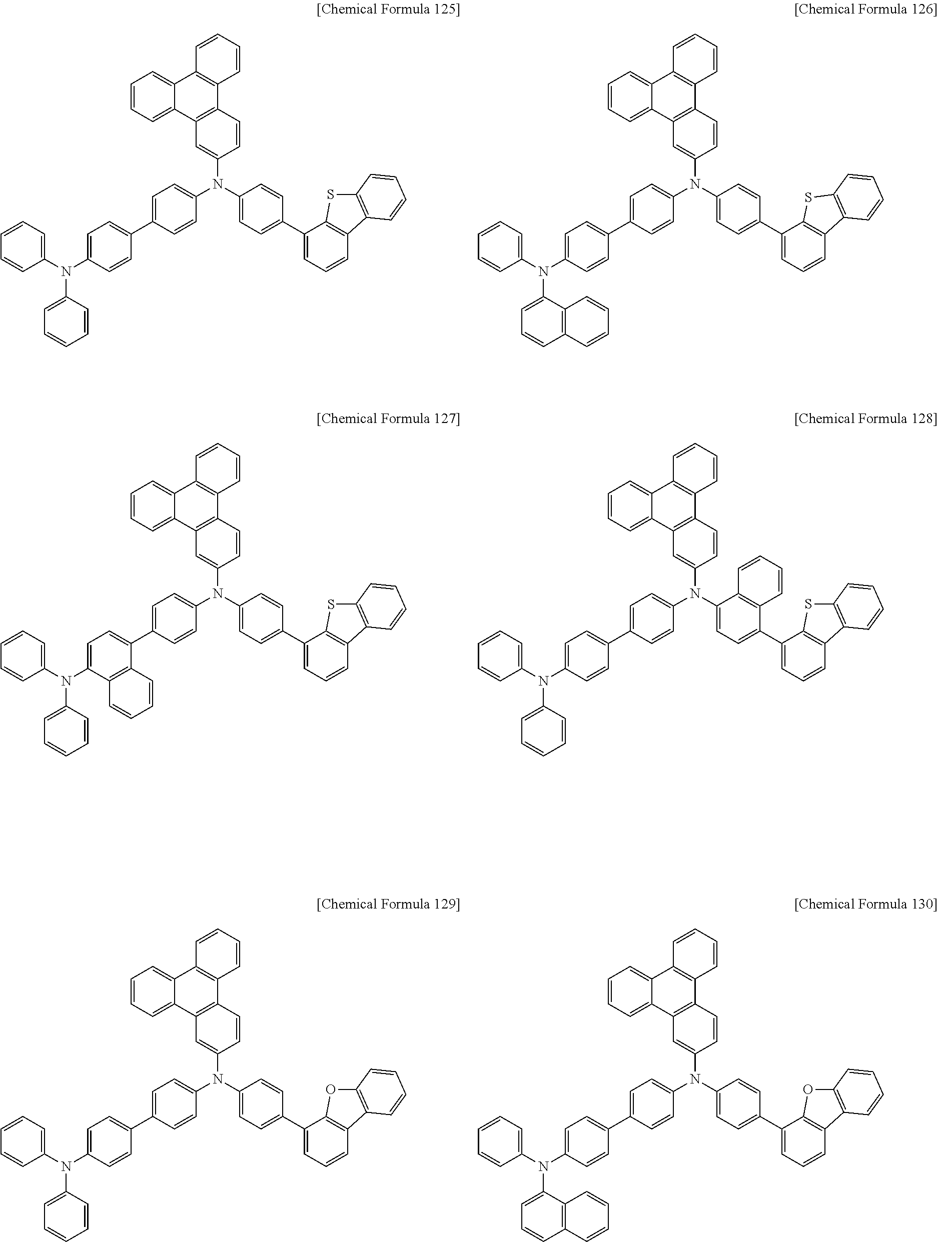

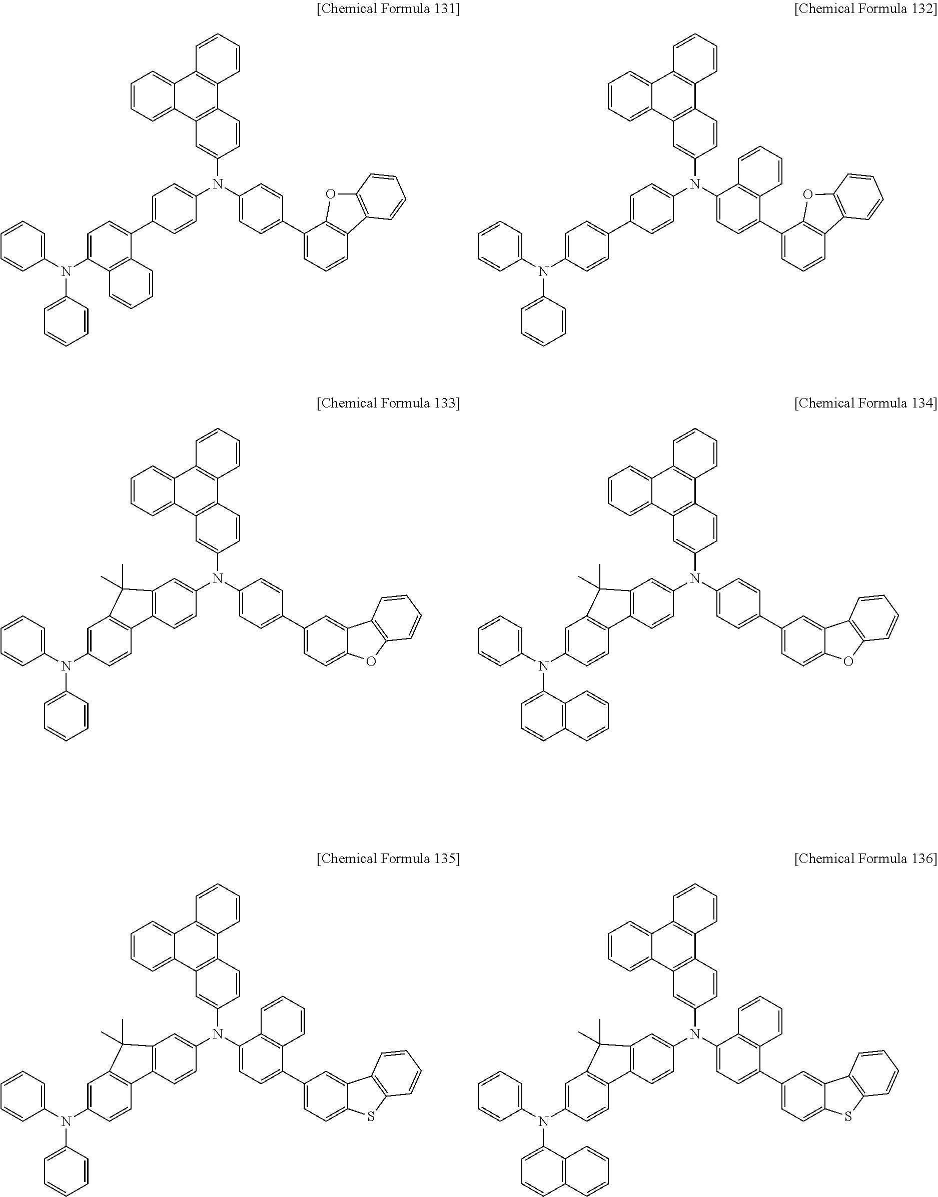

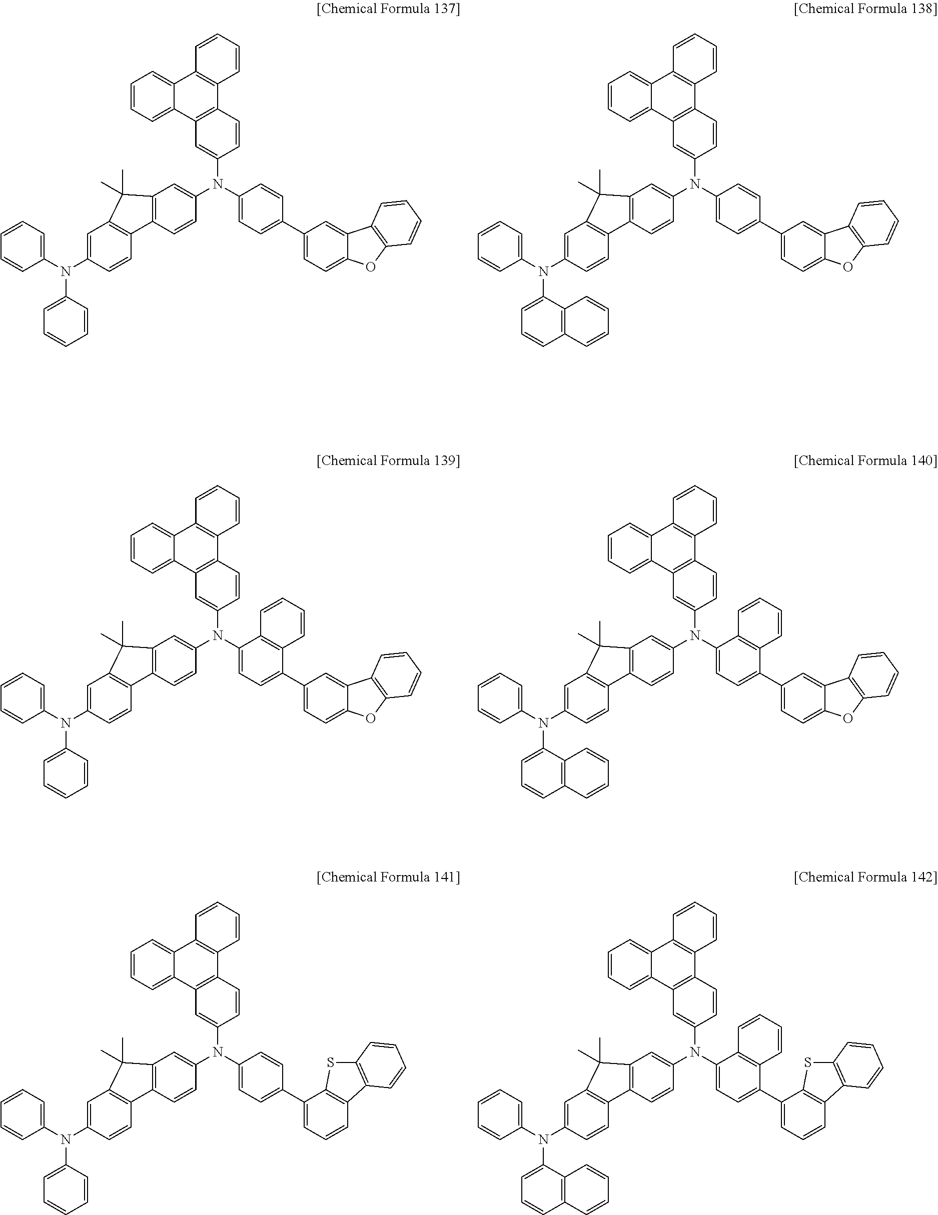

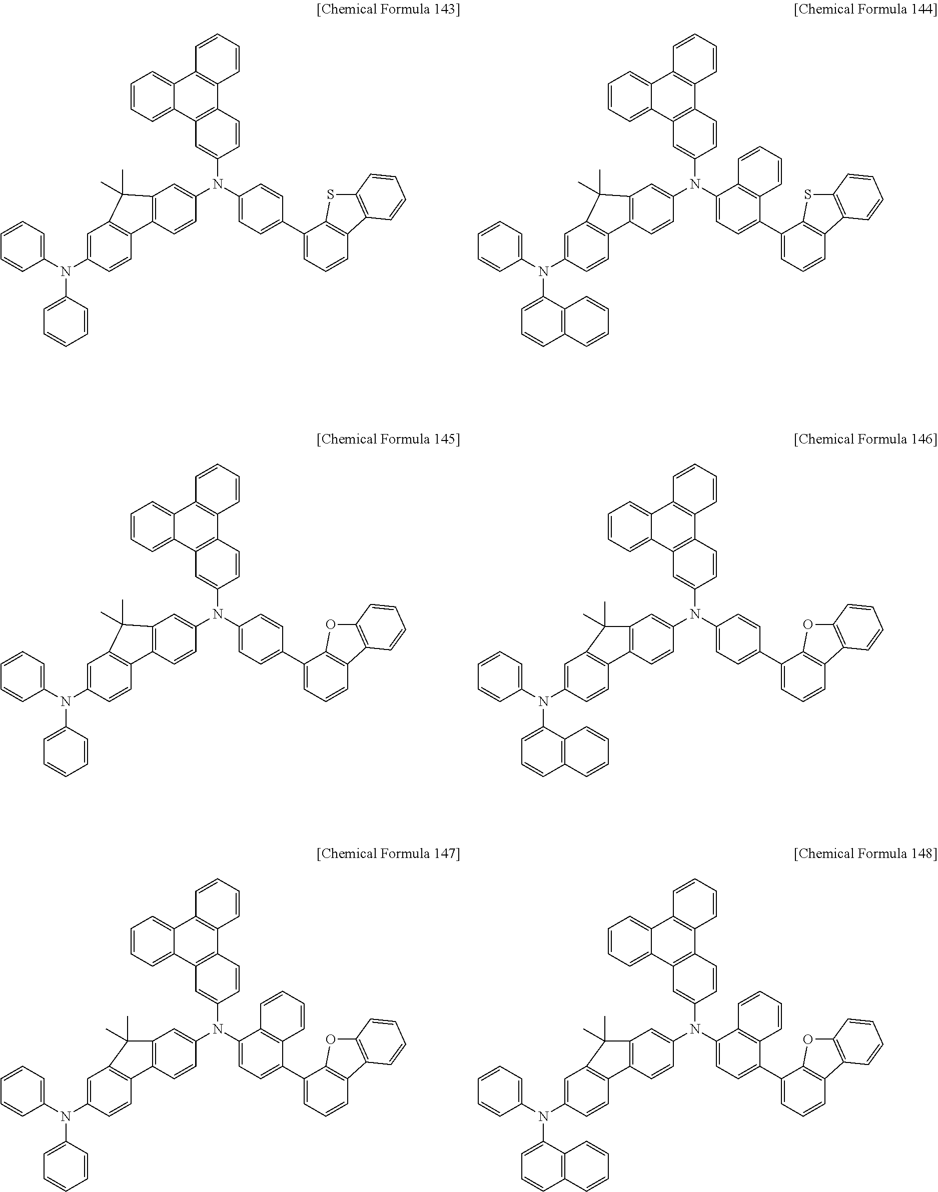

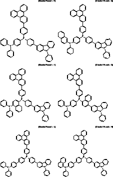

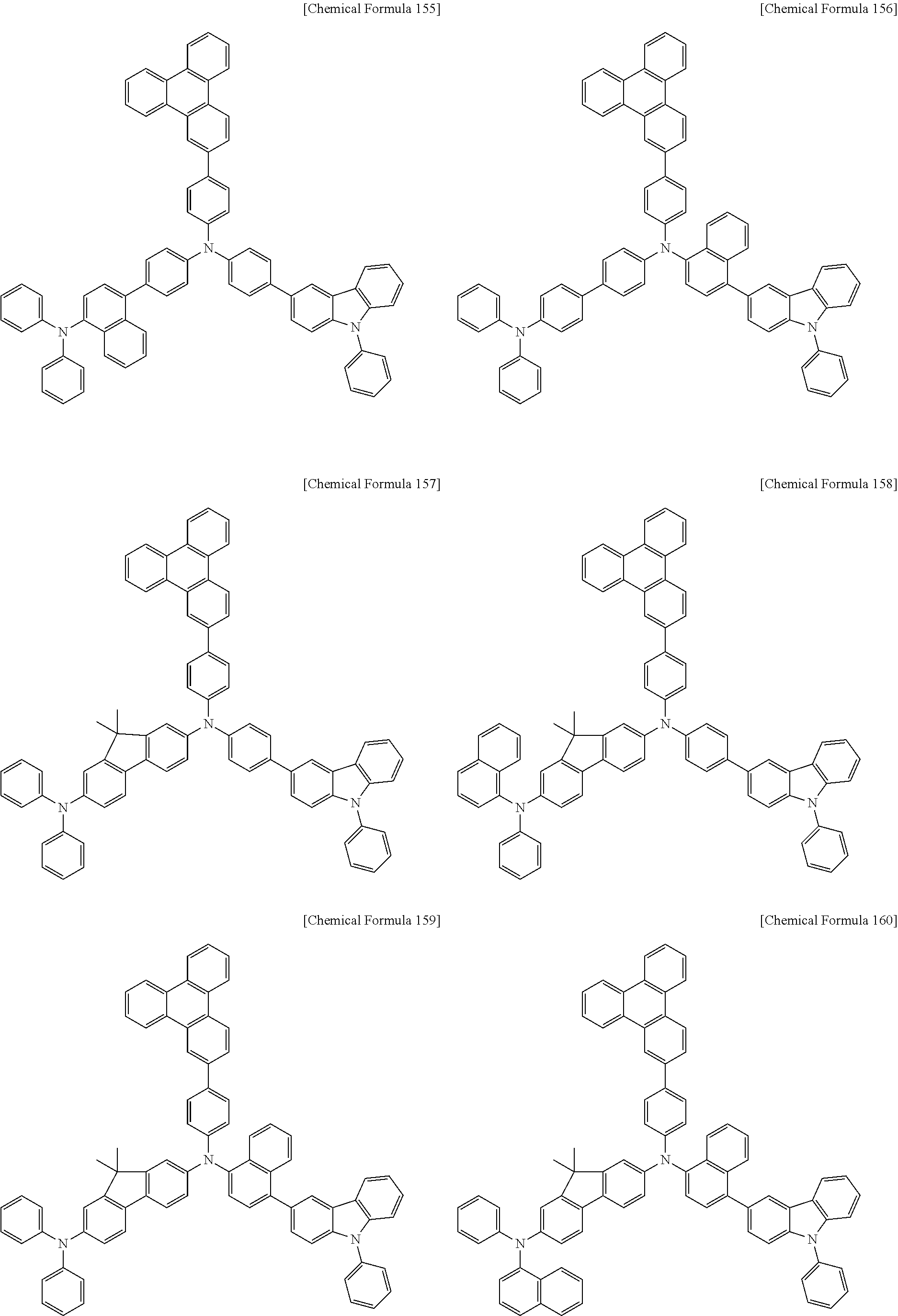

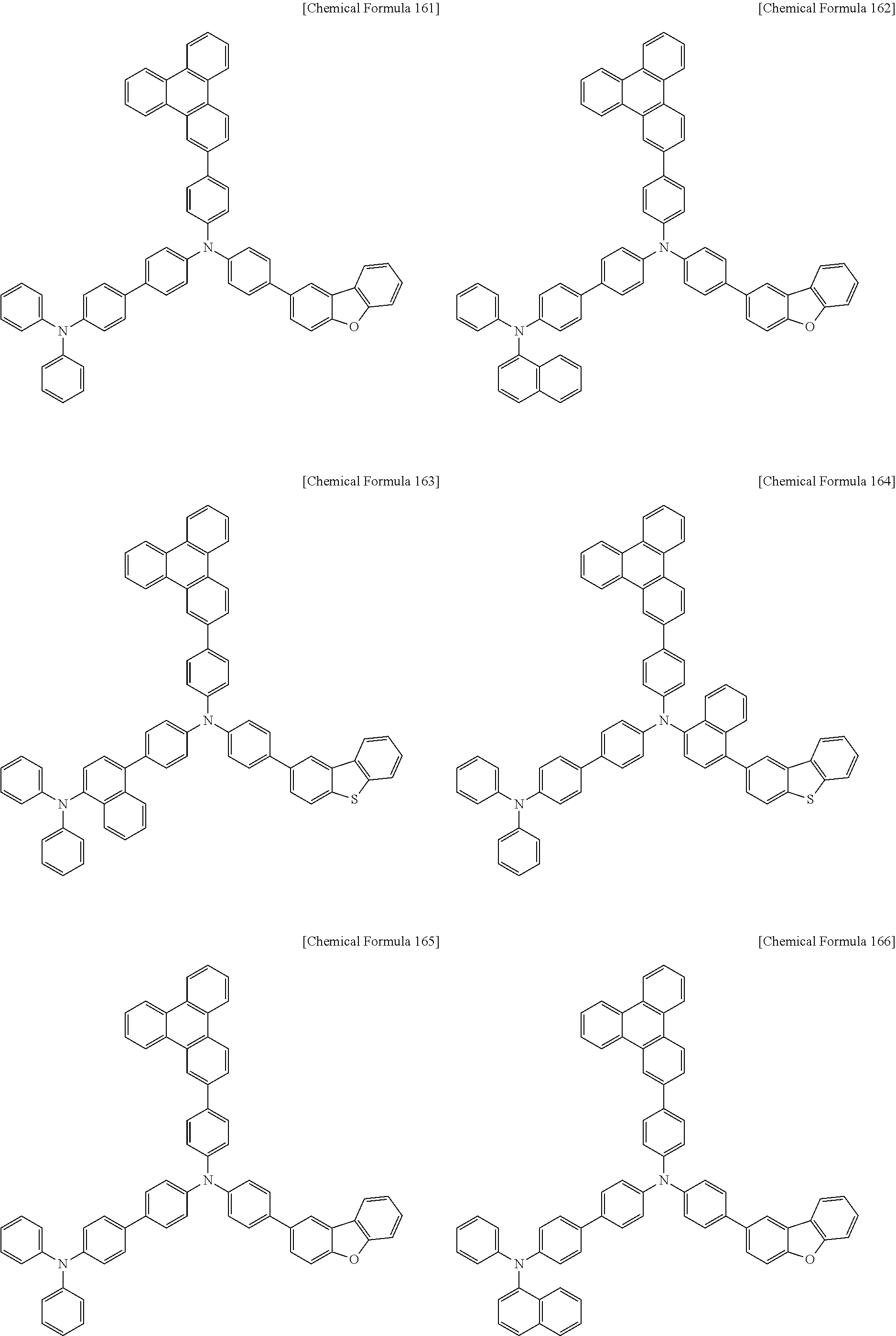

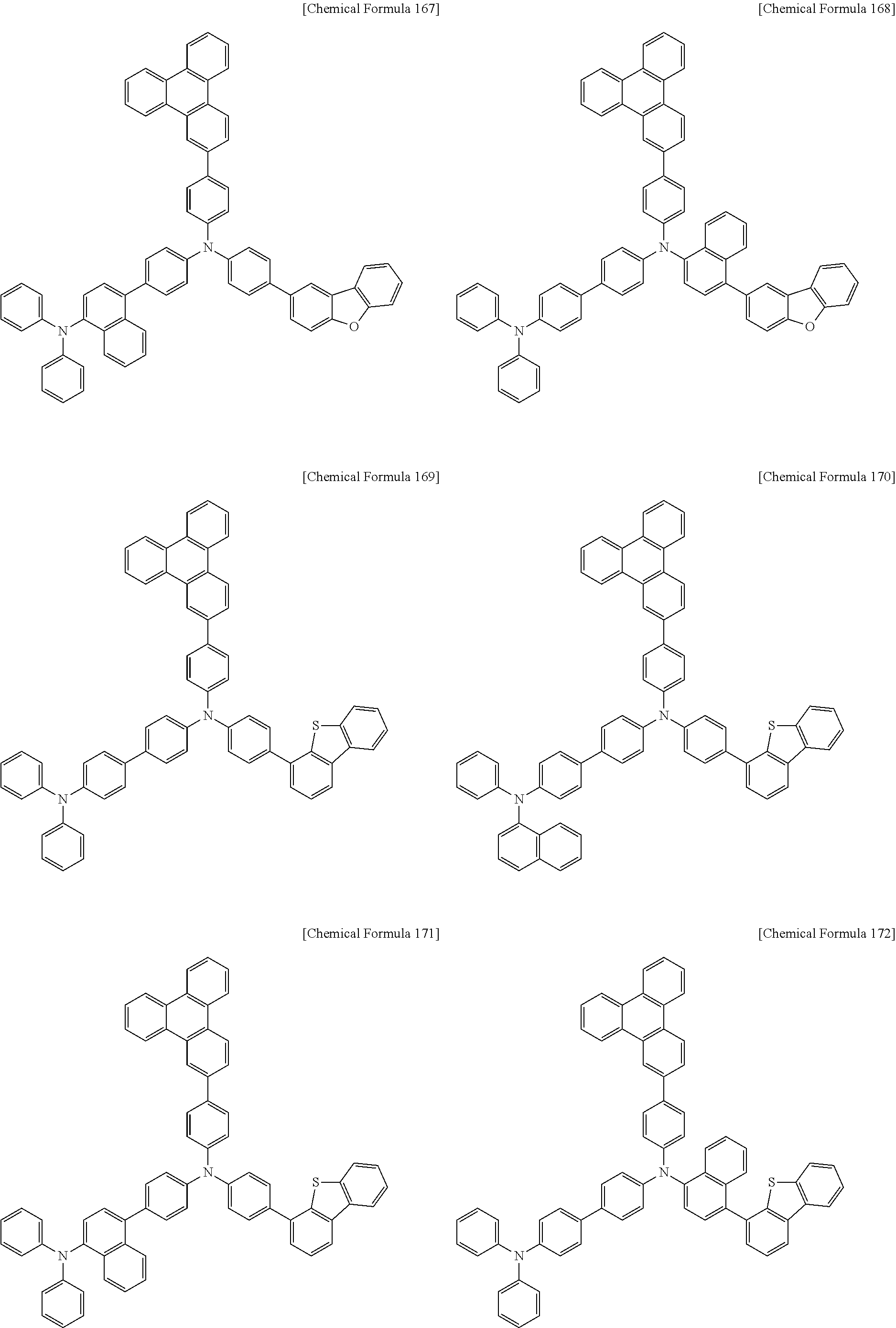

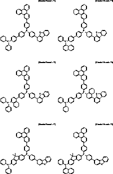

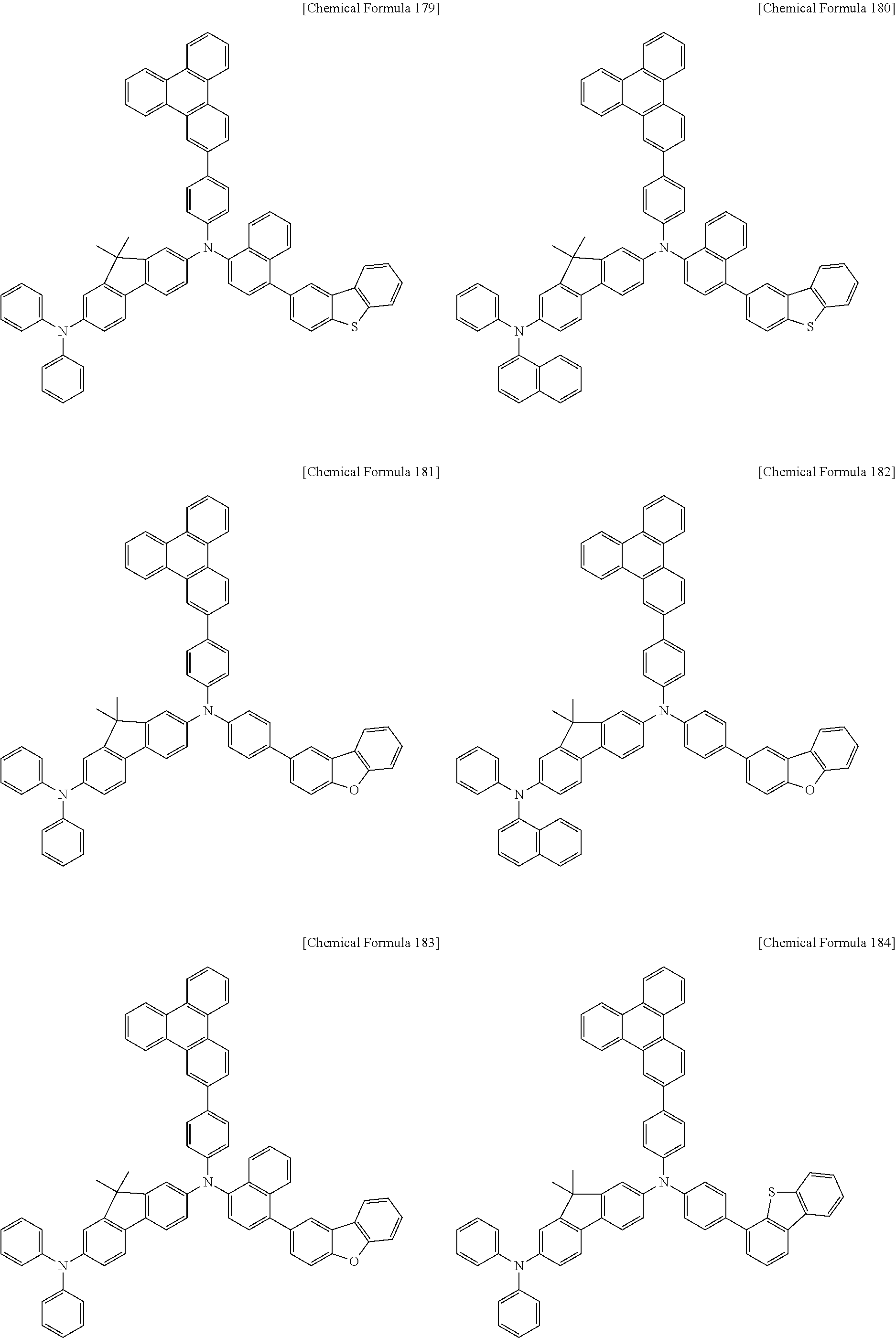

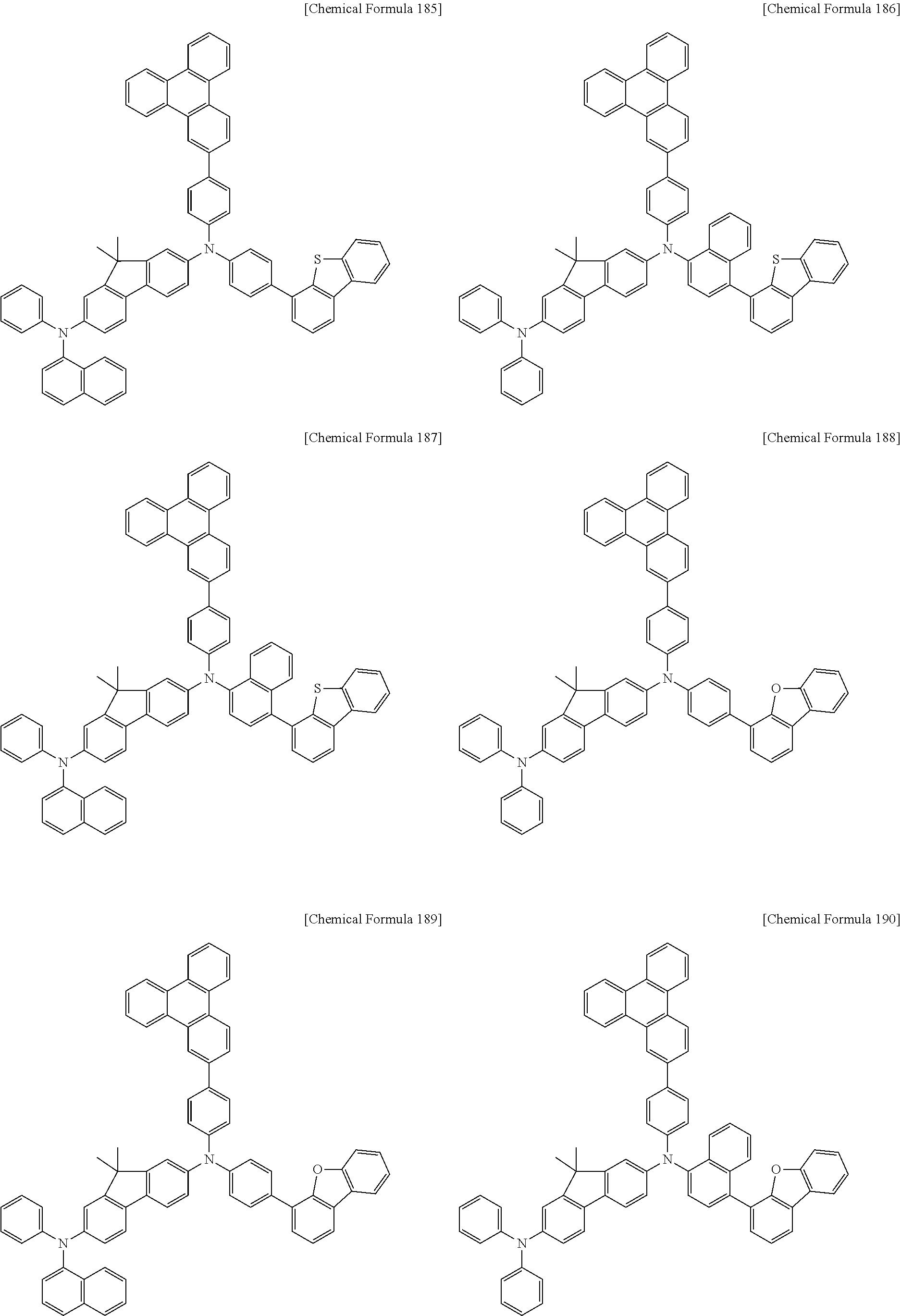

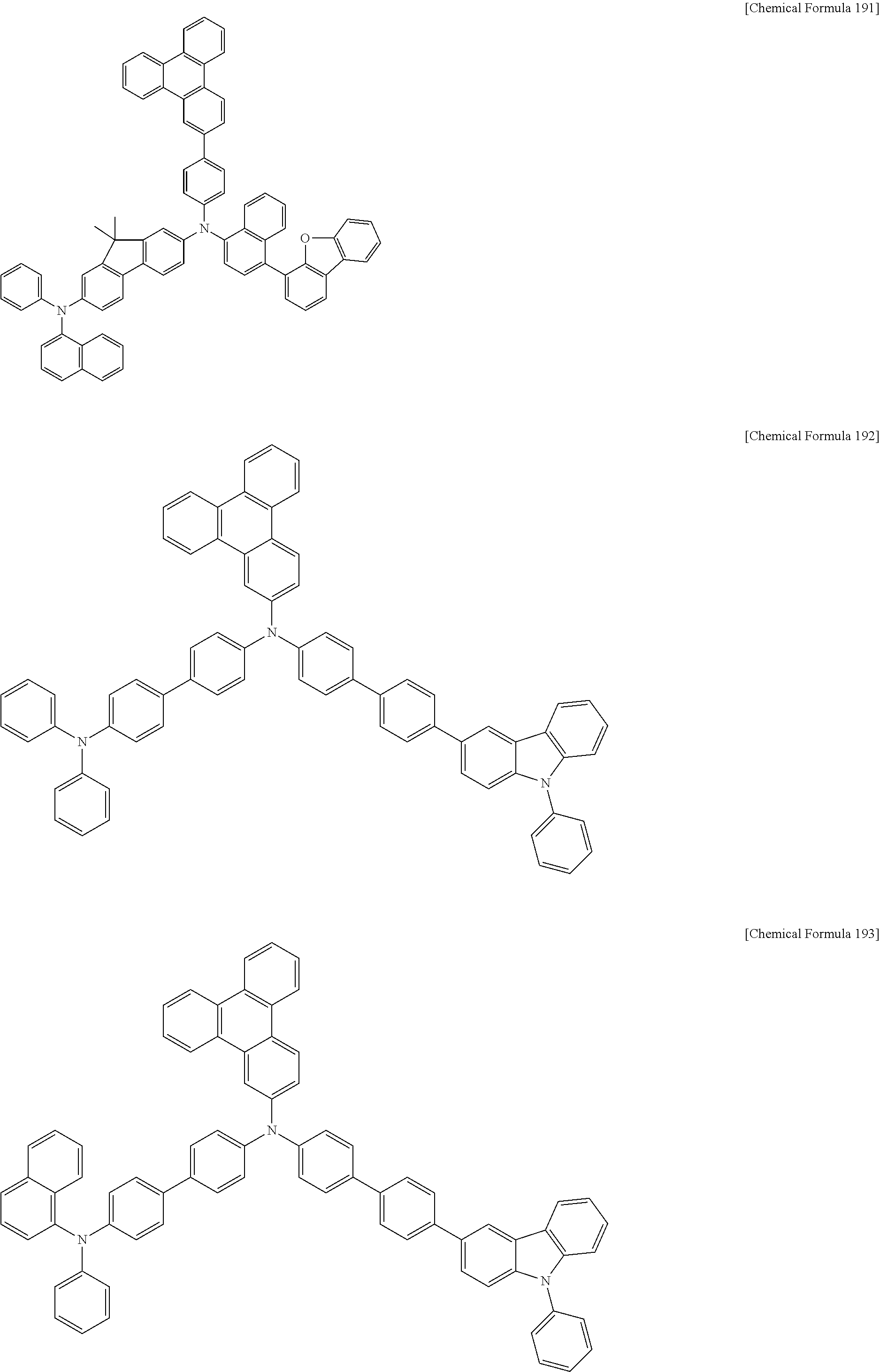 [SMILES: CC1(C)c2cc(N(c3ccccc3)c3cccc4ccccc34)ccc2-c2ccc(N(c3ccc(-c4ccc5c6ccccc6c6ccccc6c5c4)cc3)c3ccc(-c4cccc5c4oc4ccccc45)c4ccccc34)cc21 ; c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)cc2)c2cccc3ccccc23)cc1]

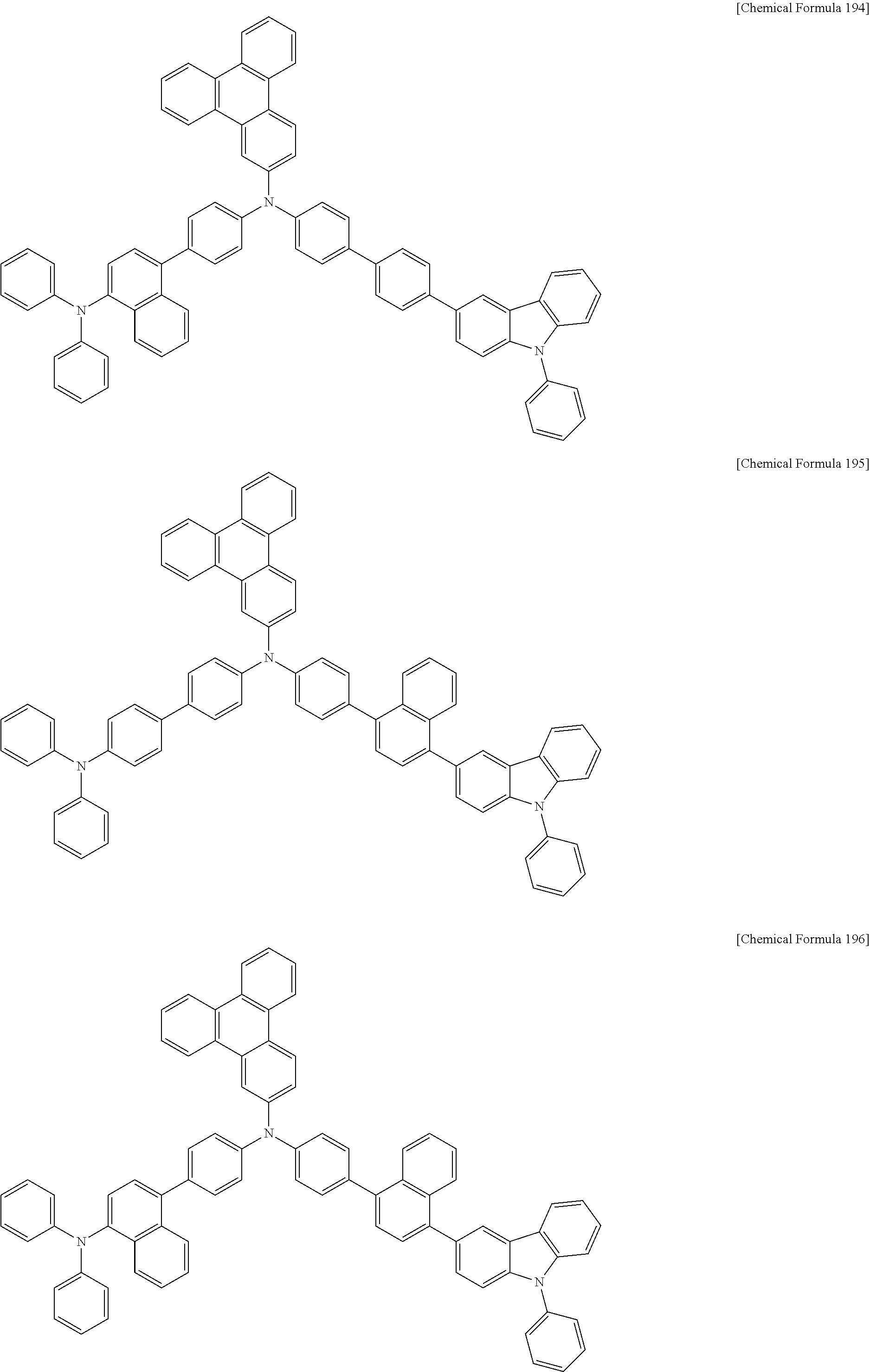 [SMILES: c1ccc(N(c2ccccc2)c2ccc(-c3ccc(N(c4ccc(-c5ccc(-c6ccc7c(c6)c6ccccc6n7-c6ccccc6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)cc2)cc1 ; c1ccc(N(c2ccccc2)c2ccc(-c3ccc(N(c4ccc(-c5ccc(-c6ccc7c(c6)c6ccccc6n7-c6ccccc6)c6ccccc56)cc4)c4ccc5c6ccccc6c6ccccc6c5c4)cc3)c3ccccc23)cc1]

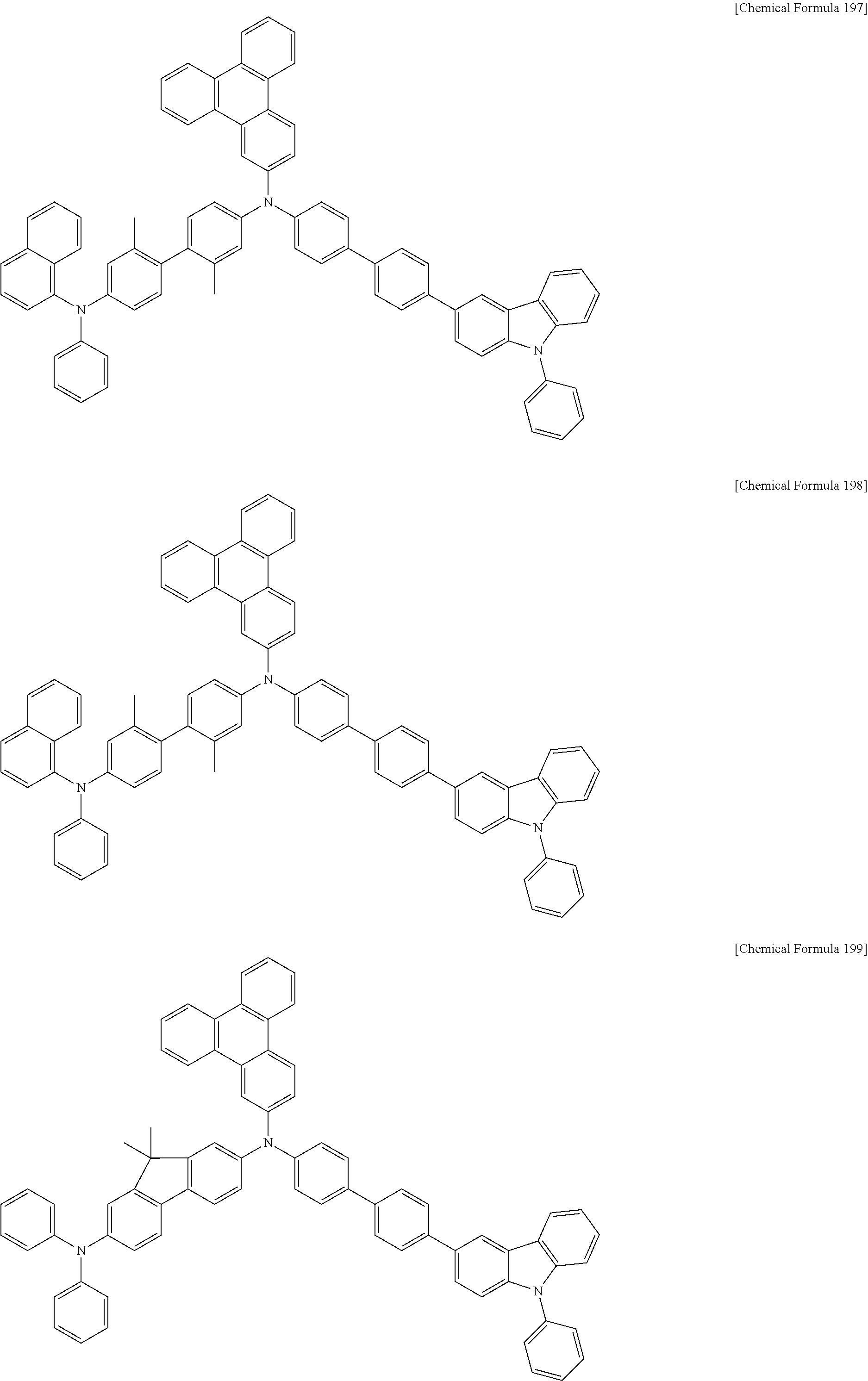 [SMILES: Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5c(c4)c4ccccc4n5-c4ccccc4)cc3)cc2)c2ccc3c4ccccc4c4ccccc4c3c2)ccc1-c1ccc(N(c2ccccc2)c2cccc3ccccc23)cc1C ; CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc4c5ccccc5c5ccccc5c4c3)cc21]

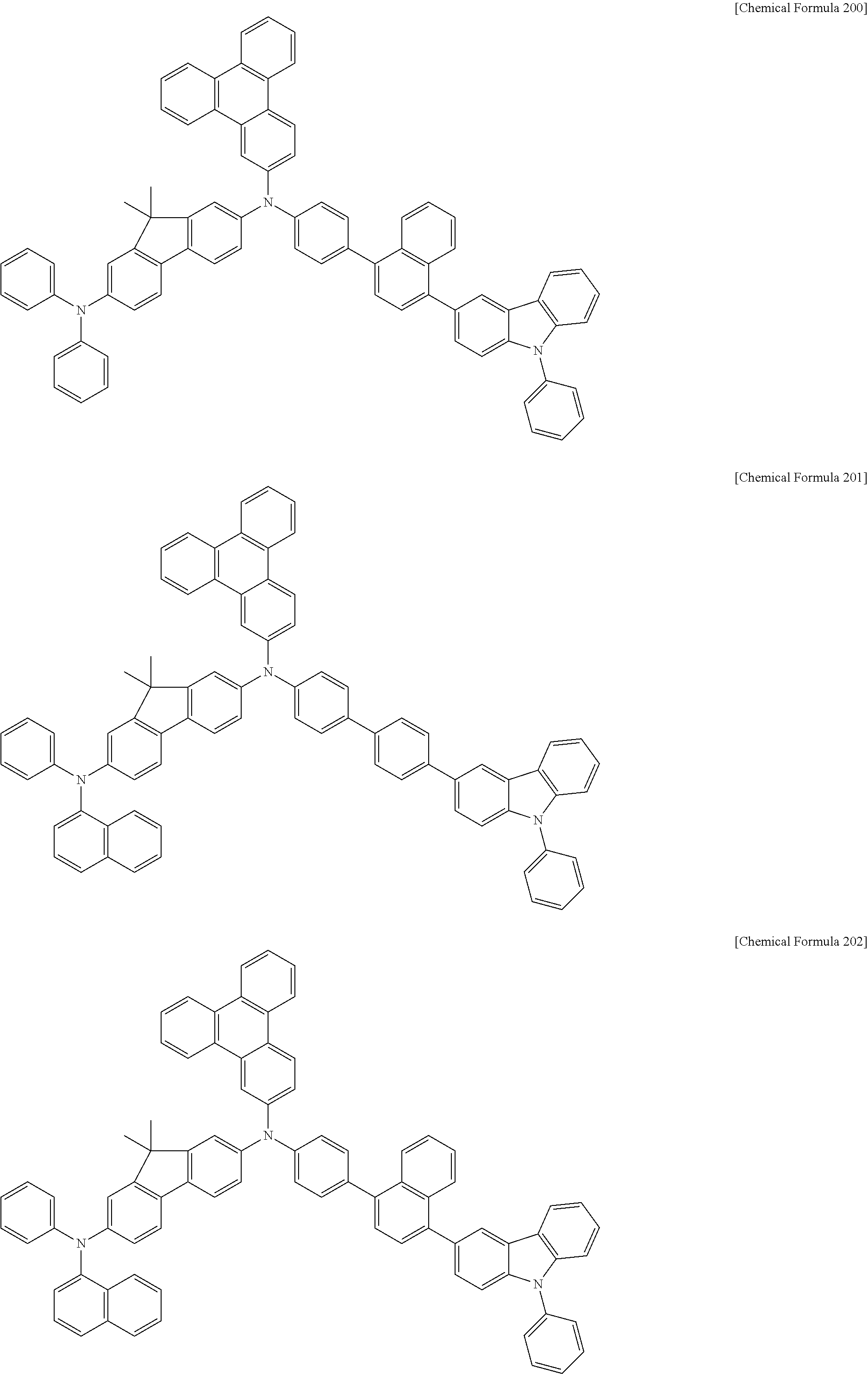 [SMILES: CC1(C)c2cc(N(c3ccccc3)c3ccccc3)ccc2-c2ccc(N(c3ccc(-c4ccc(-c5ccc6c(c5)c5ccccc5n6-c5ccccc5)c5ccccc45)cc3)c3ccc4c5ccccc5c5ccccc5c4c3)cc21 ; CC1(C)c2cc(N(c3ccc(-c4ccc(-c5ccc6c(c5)c5ccccc5n6-c5ccccc5)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccc(-c4ccc(-c5ccc6c(c5)c5ccccc5n6-c5ccccc5)c5ccccc45)cc3)c3ccc4c5ccccc5c5ccccc5c4c3)ccc2-c2ccc(N(c3ccccc3)c3cccc4ccccc34)cc21]

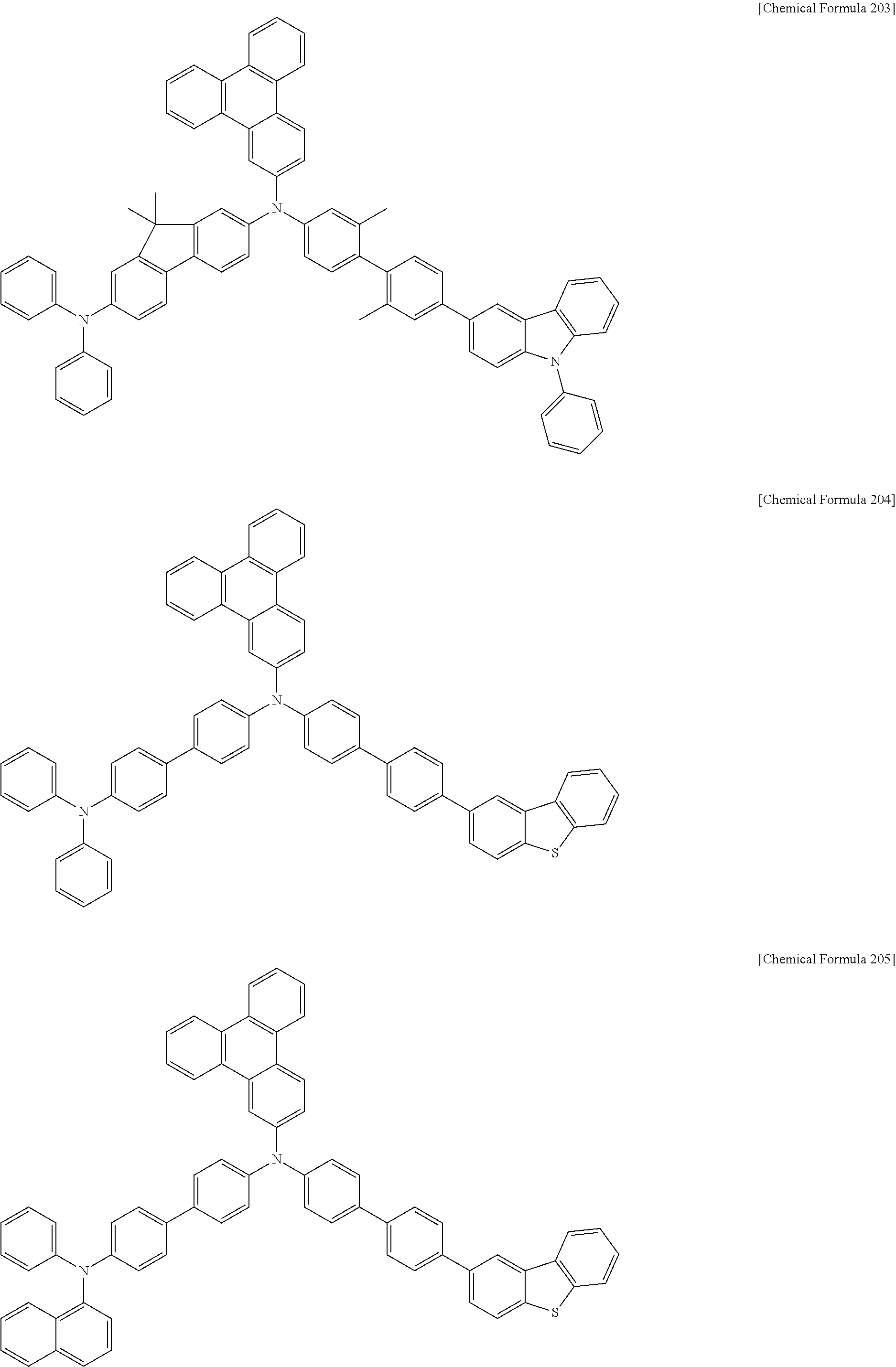 [SMILES: Cc1cc(-c2ccc3c(c2)c2ccccc2n3-c2ccccc2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7sc8ccccc8c7c6)cc5)cc4)c4ccc5c6ccccc6c6ccccc6c5c4)cc3)cc2)c2cccc3ccccc23)cc1]

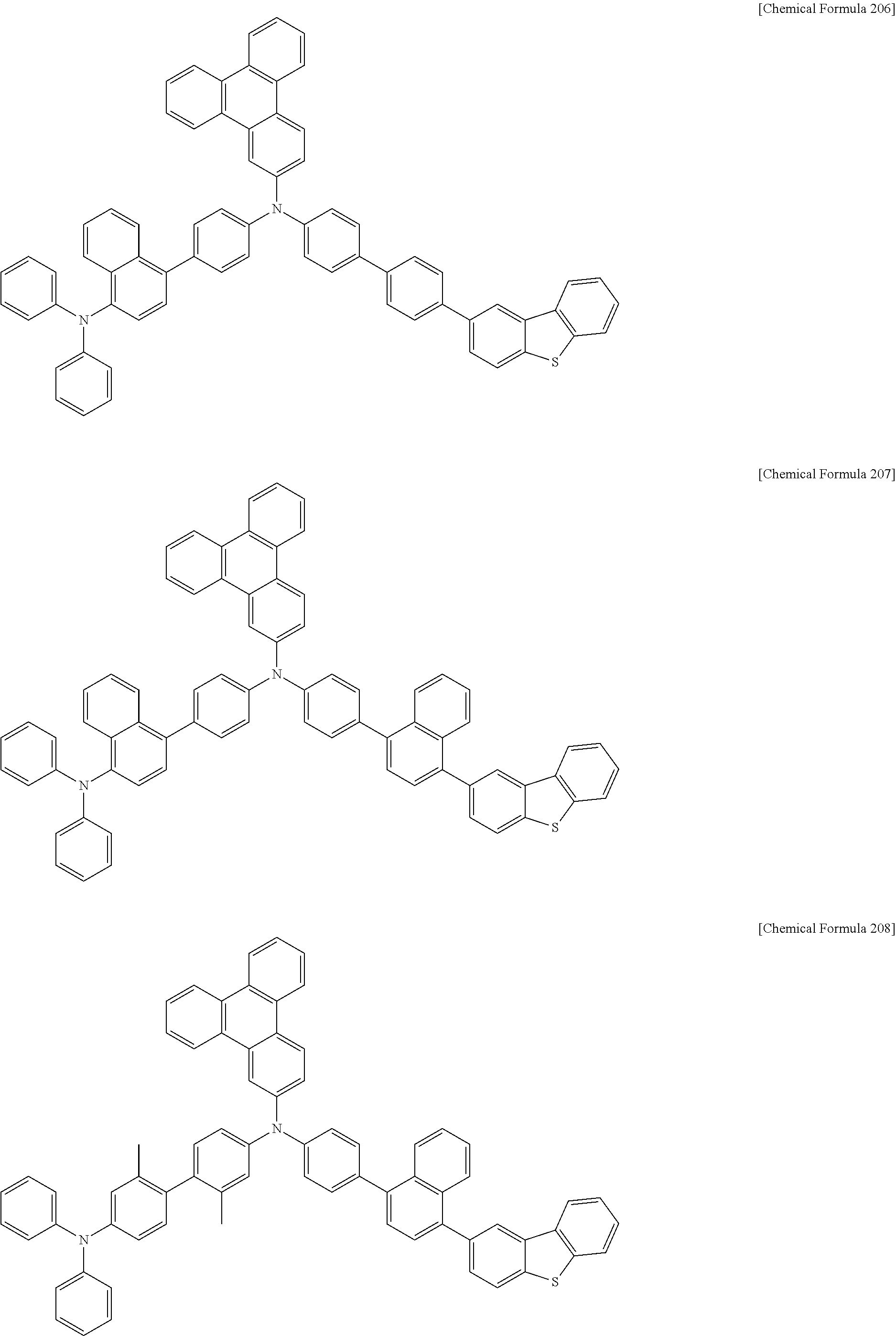 [SMILES: c1ccc(N(c2ccccc2)c2ccc(-c3ccc(N(c4ccc(-c5ccc(-c6ccc7sc8ccccc8c7c6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7sc8ccccc8c7c6)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4ccc5sc6ccccc6c5c4)c4ccccc34)cc2)c2ccc3c4ccccc4c4ccccc4c3c2)cc1C]

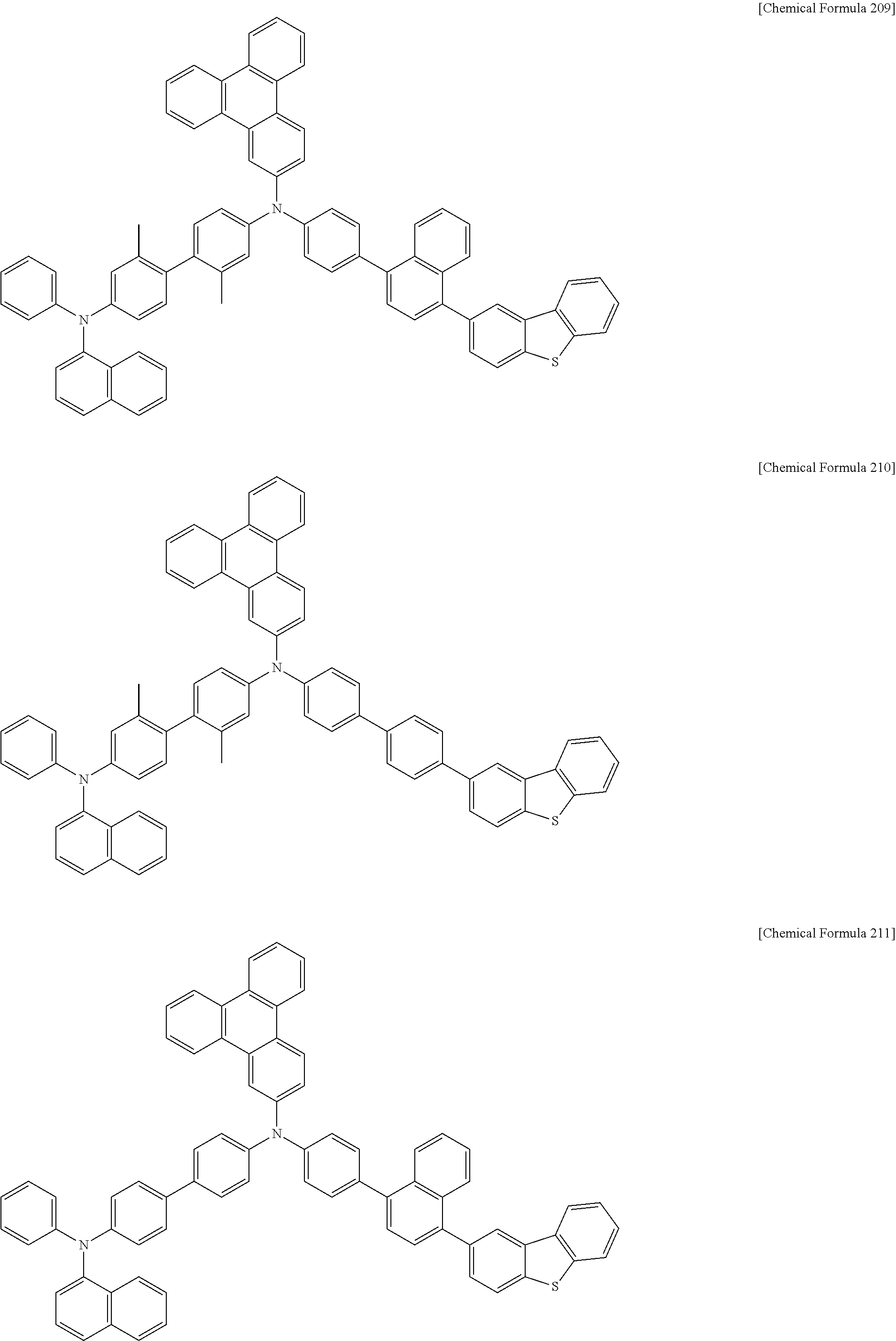 [SMILES: Cc1cc(N(c2ccc(-c3ccc(-c4ccc5sc6ccccc6c5c4)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5sc6ccccc6c5c4)cc3)cc2)c2ccc3c4ccccc4c4ccccc4c3c2)ccc1-c1ccc(N(c2ccccc2)c2cccc3ccccc23)cc1C ; c1ccc(N(c2ccc(-c3ccc(N(c4ccc(-c5ccc(-c6ccc7sc8ccccc8c7c6)c6ccccc56)cc4)c4ccc5c6ccccc6c6ccccc6c5c4)cc3)cc2)c2cccc3ccccc23)cc1]

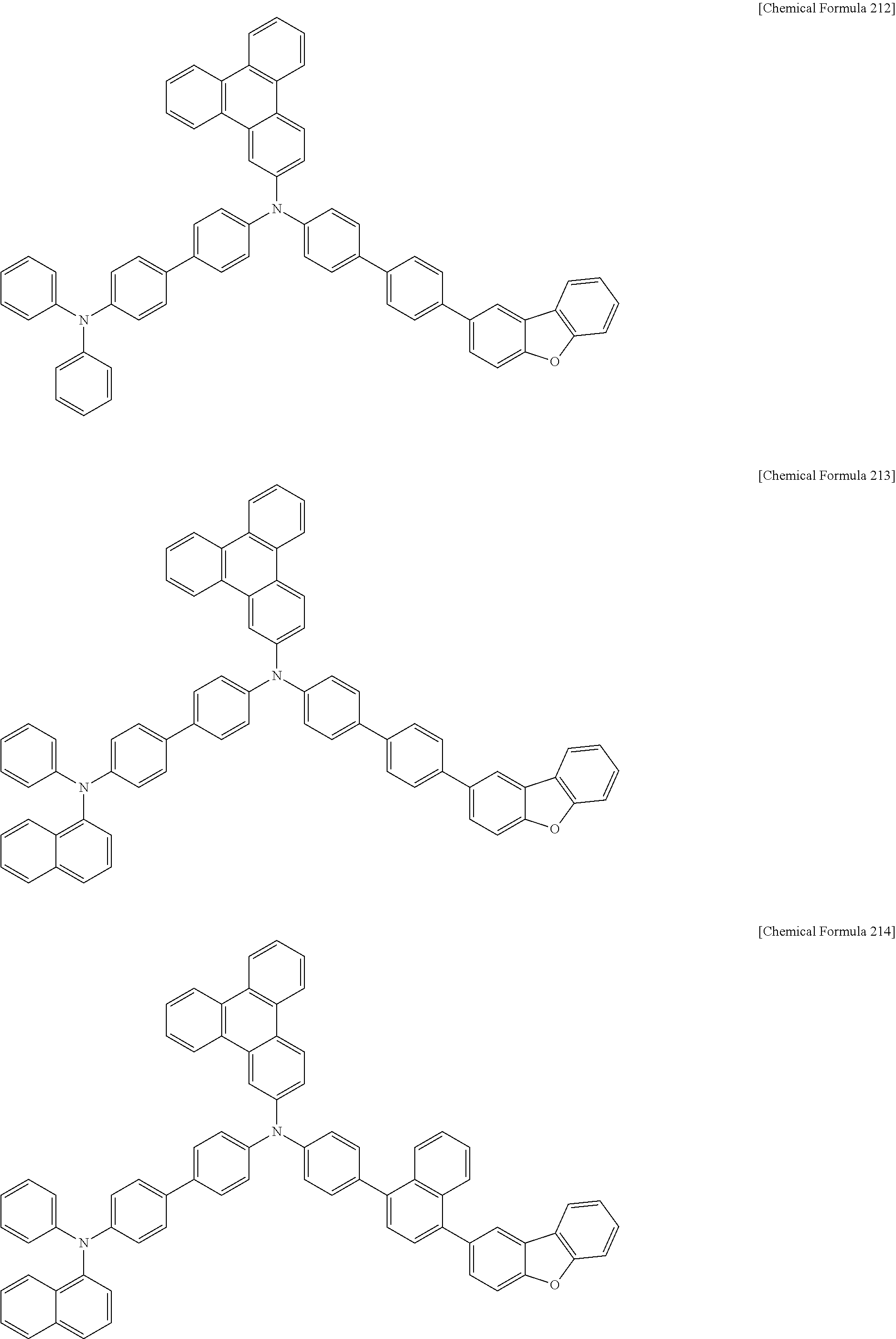 [SMILES: c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)cc2)cc1 ; c1ccc(N(c2ccc(-c3ccc(N(c4ccc(-c5ccc(-c6ccc7oc8ccccc8c7c6)cc5)cc4)c4ccc5c6ccccc6c6ccccc6c5c4)cc3)cc2)c2cccc3ccccc23)cc1 ; c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1]

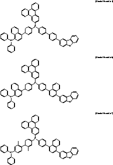 [SMILES: c1ccc(N(c2ccccc2)c2ccc(-c3ccc(N(c4ccc(-c5ccc(-c6ccc7oc8ccccc8c7c6)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6ccc7oc8ccccc8c7c6)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4ccc5oc6ccccc6c5c4)c4ccccc34)cc2)c2ccc3c4ccccc4c4ccccc4c3c2)cc1C]

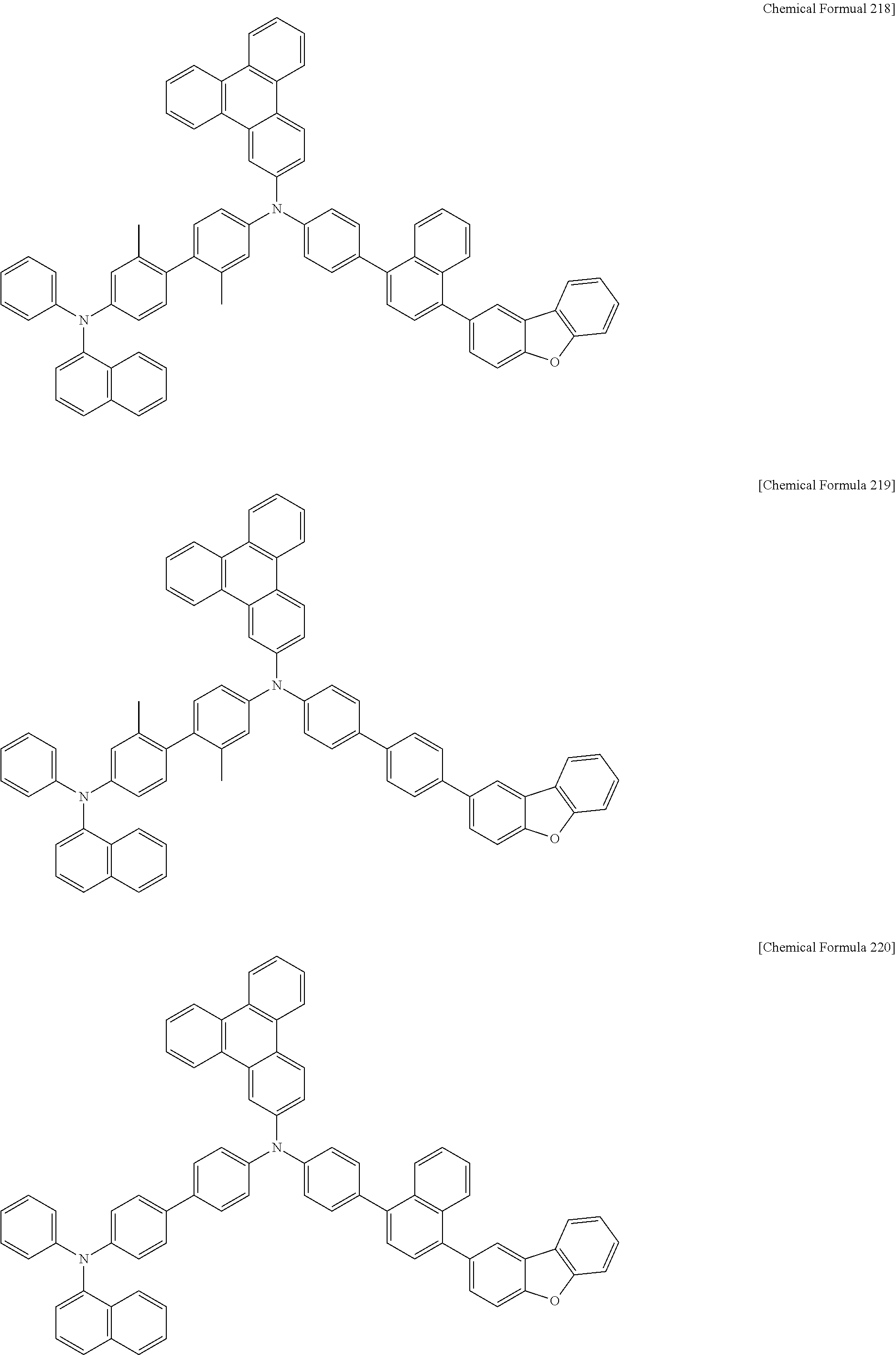 [SMILES: Cc1cc(N(c2ccc(-c3ccc(-c4ccc5oc6ccccc6c5c4)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4ccc5oc6ccccc6c5c4)cc3)cc2)c2ccc3c4ccccc4c4ccccc4c3c2)ccc1-c1ccc(N(c2ccccc2)c2cccc3ccccc23)cc1C ; c1ccc(N(c2ccc(-c3ccc(N(c4ccc(-c5ccc(-c6ccc7oc8ccccc8c7c6)c6ccccc56)cc4)c4ccc5c6ccccc6c6ccccc6c5c4)cc3)cc2)c2cccc3ccccc23)cc1]

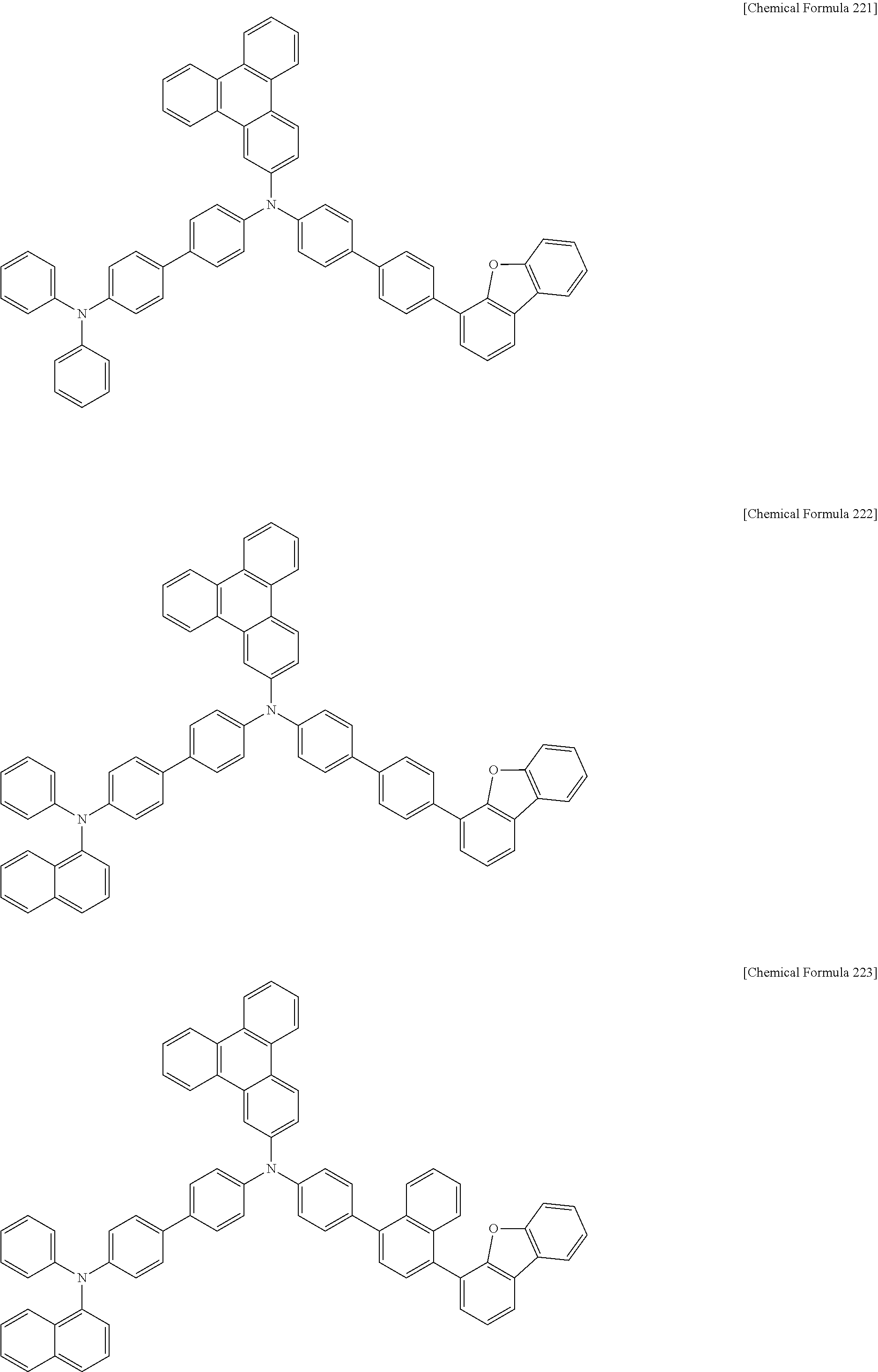 [SMILES: c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)cc2)cc1 ; c1ccc(N(c2ccc(-c3ccc(N(c4ccc(-c5ccc(-c6cccc7c6oc6ccccc67)cc5)cc4)c4ccc5c6ccccc6c6ccccc6c5c4)cc3)cc2)c2cccc3ccccc23)cc1 ; c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1]

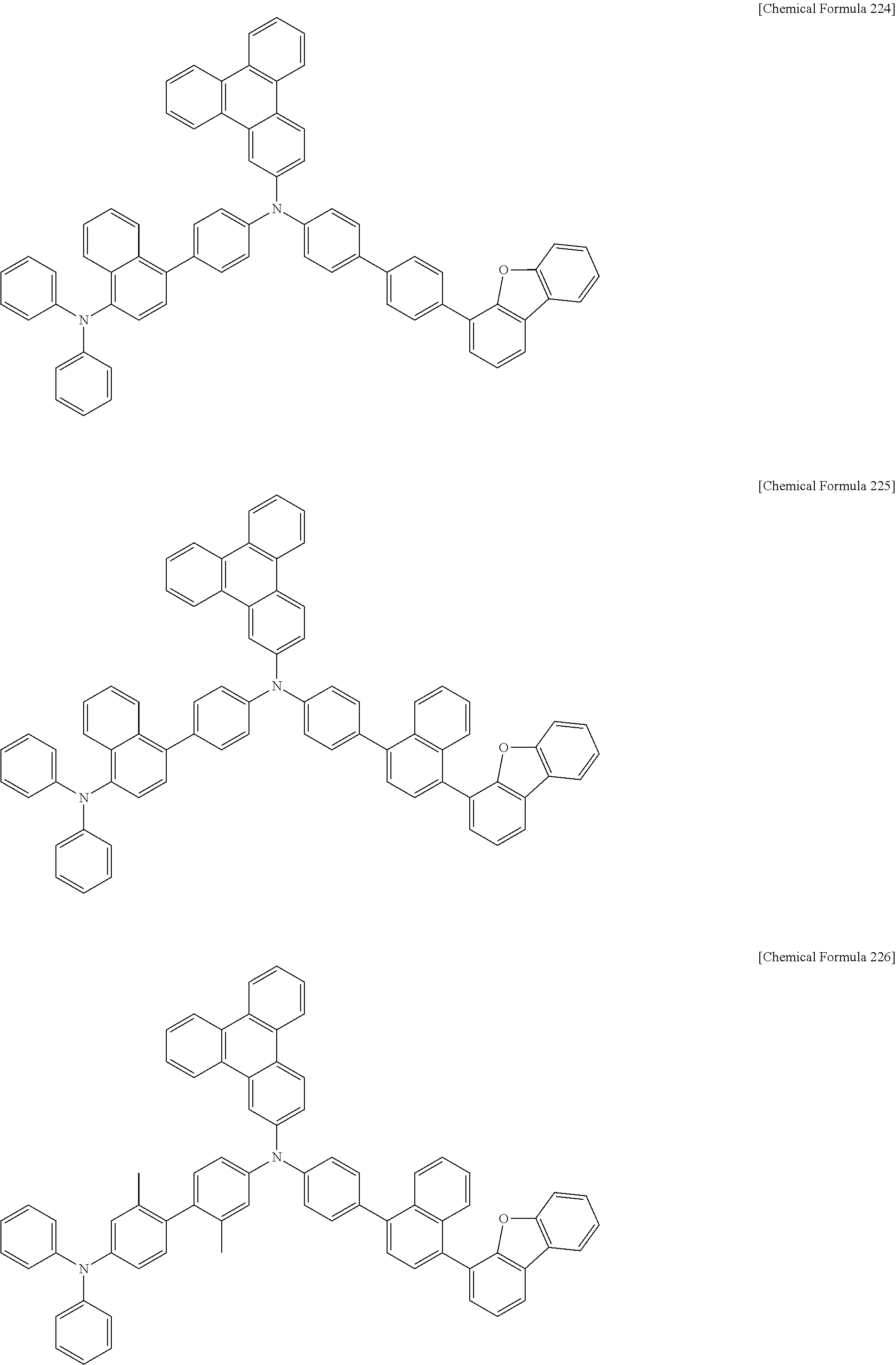 [SMILES: c1ccc(N(c2ccccc2)c2ccc(-c3ccc(N(c4ccc(-c5ccc(-c6cccc7c6oc6ccccc67)cc5)cc4)c4ccc5c6ccccc6c6ccccc6c5c4)cc3)c3ccccc23)cc1 ; c1ccc(N(c2ccccc2)c2ccc(-c3ccc(N(c4ccc(-c5ccc(-c6cccc7c6oc6ccccc67)c6ccccc56)cc4)c4ccc5c6ccccc6c6ccccc6c5c4)cc3)c3ccccc23)cc1 ; Cc1cc(N(c2ccccc2)c2ccccc2)ccc1-c1ccc(N(c2ccc(-c3ccc(-c4cccc5c4oc4ccccc45)c4ccccc34)cc2)c2ccc3c4ccccc4c4ccccc4c3c2)cc1C]

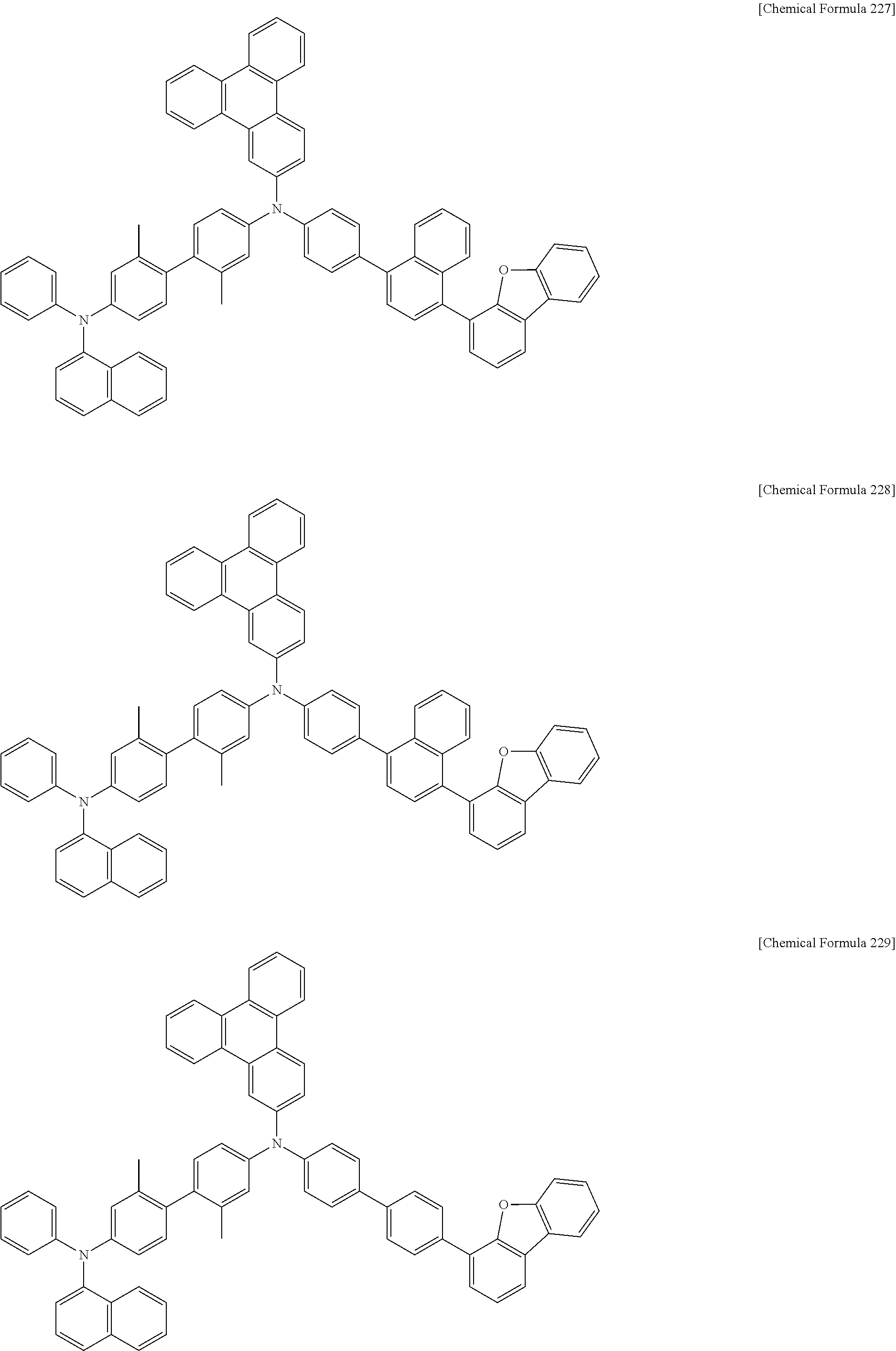 [SMILES: Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4cccc5c4oc4ccccc45)c4ccccc34)cc2)c2ccc3c4ccccc4c4ccccc4c3c2)ccc1-c1ccc(N(c2ccccc2)c2cccc3ccccc23)cc1C ; Cc1cc(N(c2ccc(-c3ccc(-c4cccc5c4oc4ccccc45)cc3)cc2)c2ccc3c4ccccc4c4ccccc4c3c2)ccc1-c1ccc(N(c2ccccc2)c2cccc3ccccc23)cc1C]

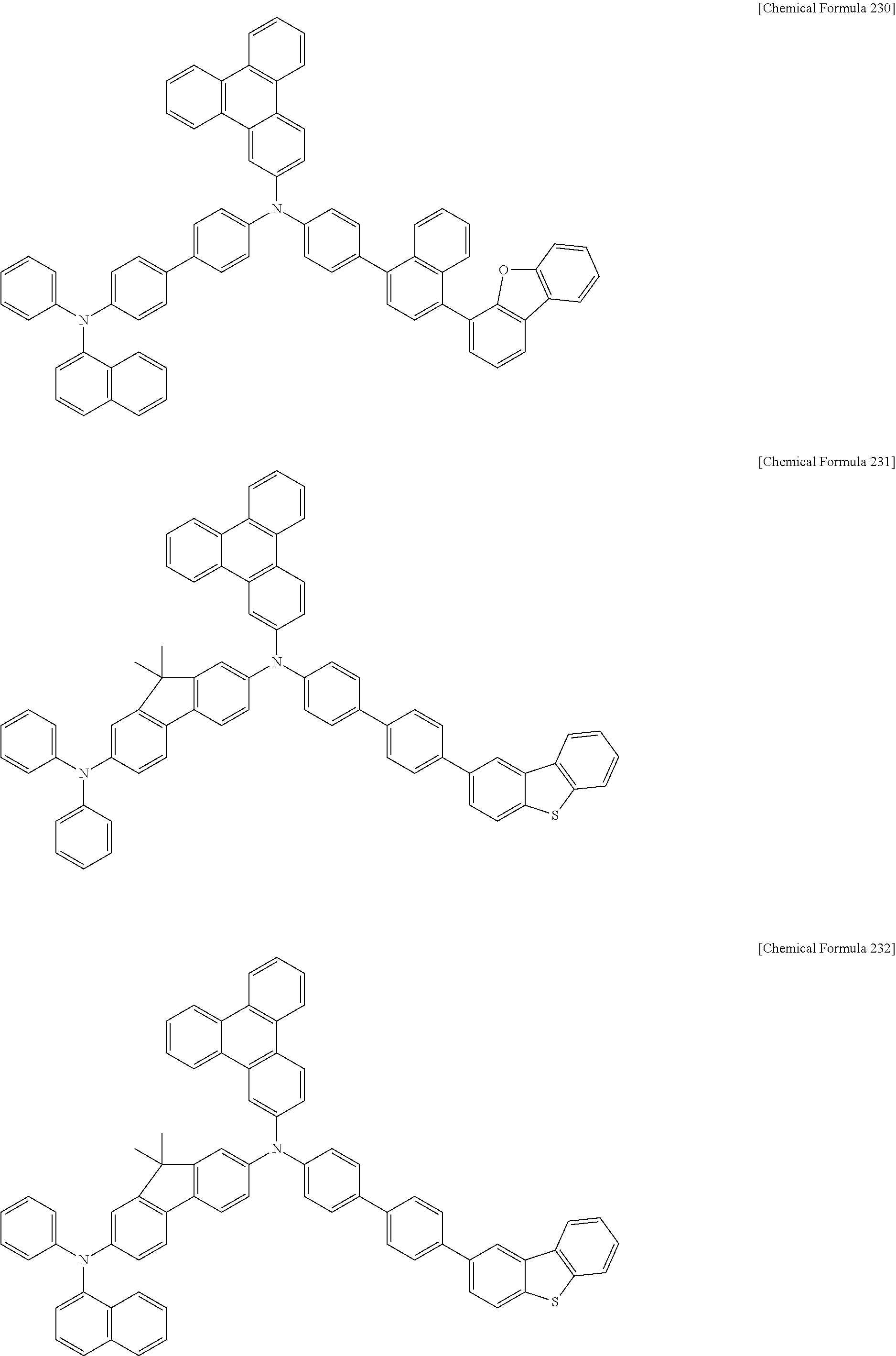 [SMILES: c1ccc(N(c2ccc(-c3ccc(N(c4ccc(-c5ccc(-c6cccc7c6oc6ccccc67)c6ccccc56)cc4)c4ccc5c6ccccc6c6ccccc6c5c4)cc3)cc2)c2cccc3ccccc23)cc1 ; CC1(C)c2cc(N(c3ccccc3)c3ccccc3)ccc2-c2ccc(N(c3ccc(-c4ccc(-c5ccc6sc7ccccc7c6c5)cc4)cc3)c3ccc4c5ccccc5c5ccccc5c4c3)cc21 ; CC1(C)c2cc(N(c3ccc(-c4ccc(-c5ccc6sc7ccccc7c6c5)cc4)cc3)c3ccc4c5ccccc5c5ccccc5c4c3)ccc2-c2ccc(N(c3ccccc3)c3cccc4ccccc34)cc21]

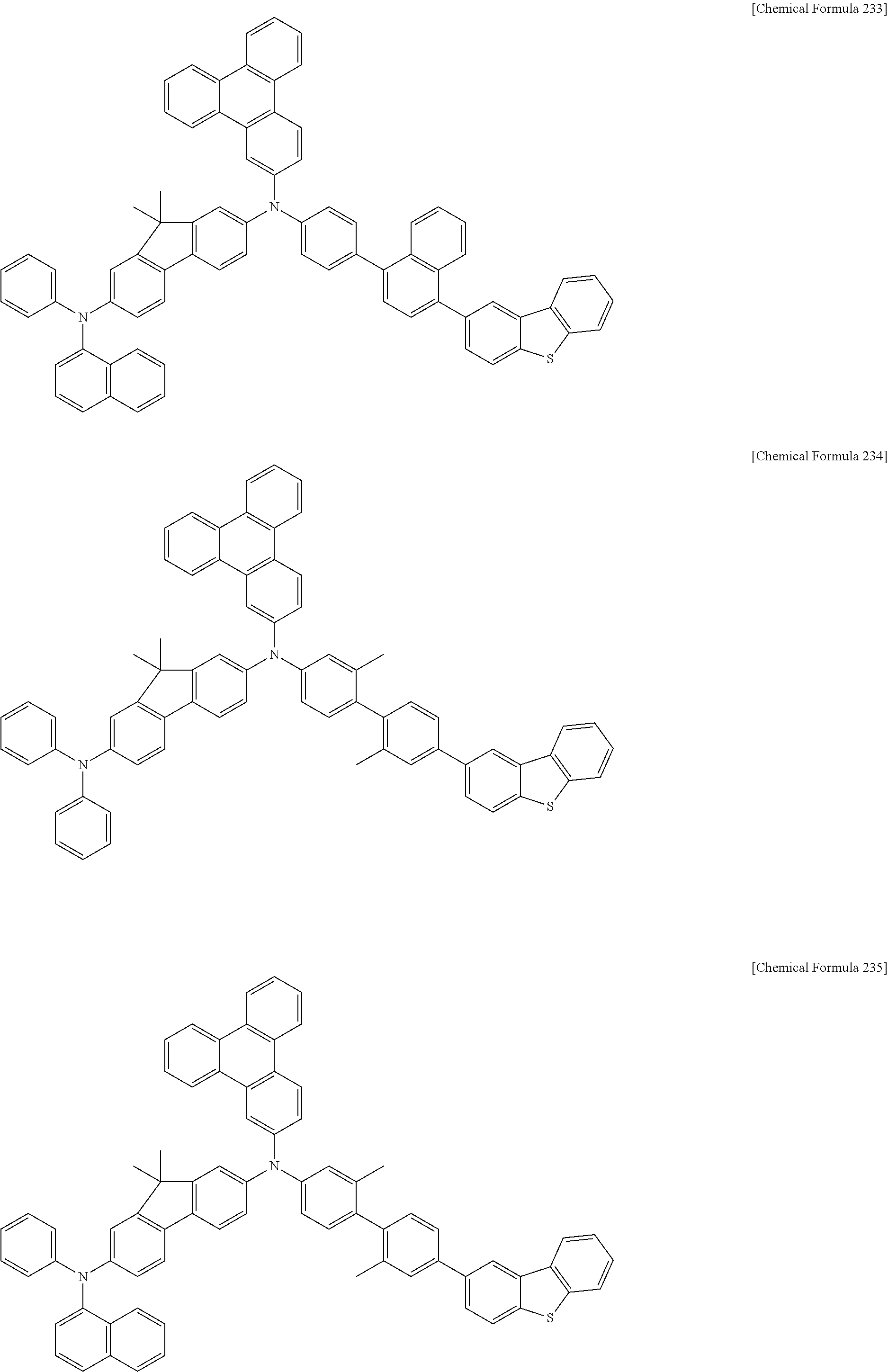 [SMILES: CC1(C)c2cc(N(c3ccc(-c4ccc(-c5ccc6sc7ccccc7c6c5)c5ccccc45)cc3)c3ccc4c5ccccc5c5ccccc5c4c3)ccc2-c2ccc(N(c3ccccc3)c3cccc4ccccc34)cc21 ; Cc1cc(-c2ccc3sc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2ccc3sc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

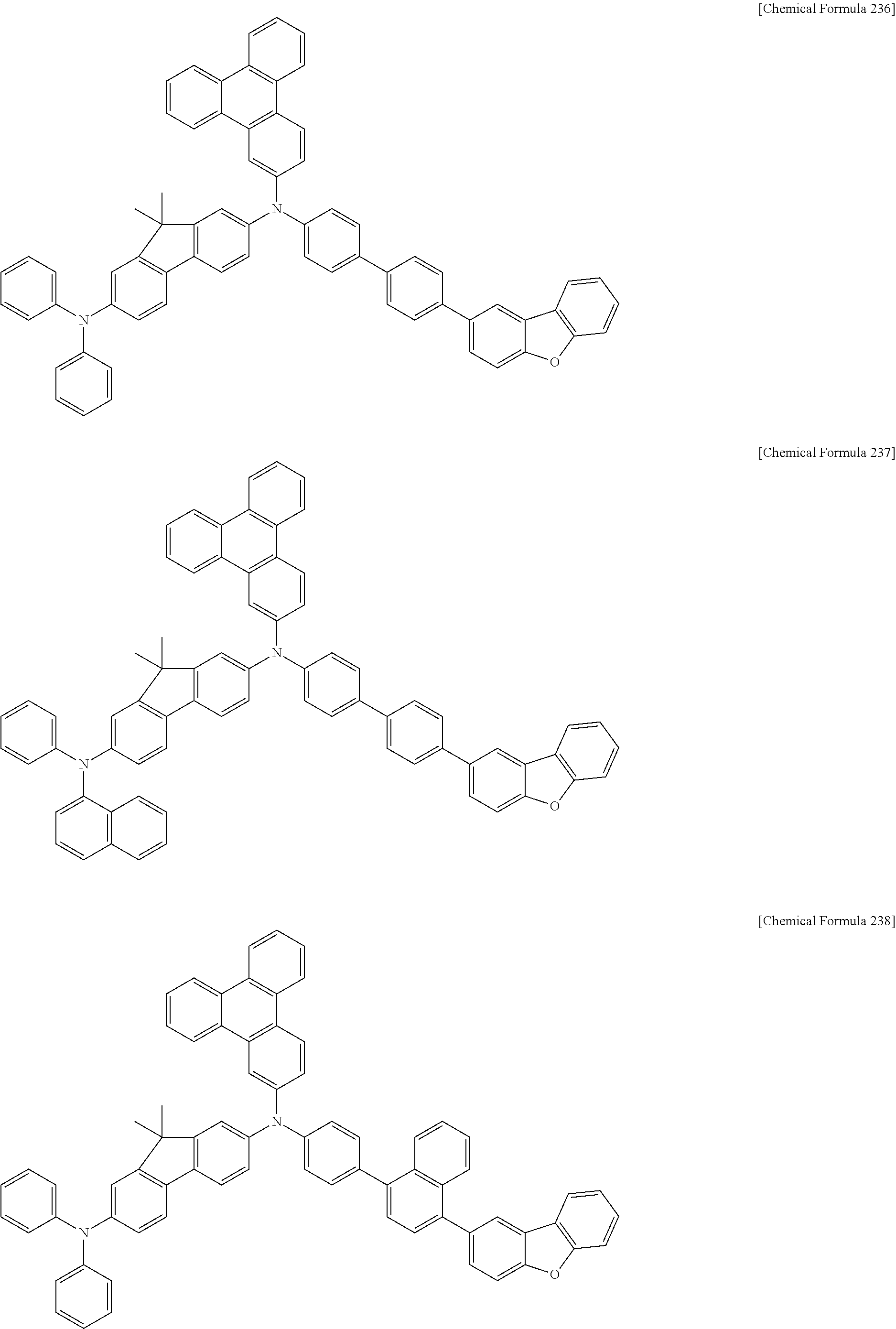 [SMILES: CC1(C)c2cc(N(c3ccccc3)c3ccccc3)ccc2-c2ccc(N(c3ccc(-c4ccc(-c5ccc6oc7ccccc7c6c5)cc4)cc3)c3ccc4c5ccccc5c5ccccc5c4c3)cc21 ; CC1(C)c2cc(N(c3ccc(-c4ccc(-c5ccc6oc7ccccc7c6c5)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5ccc6oc7ccccc7c6c5)c5ccccc45)cc3)c3ccc4c5ccccc5c5ccccc5c4c3)cc21]

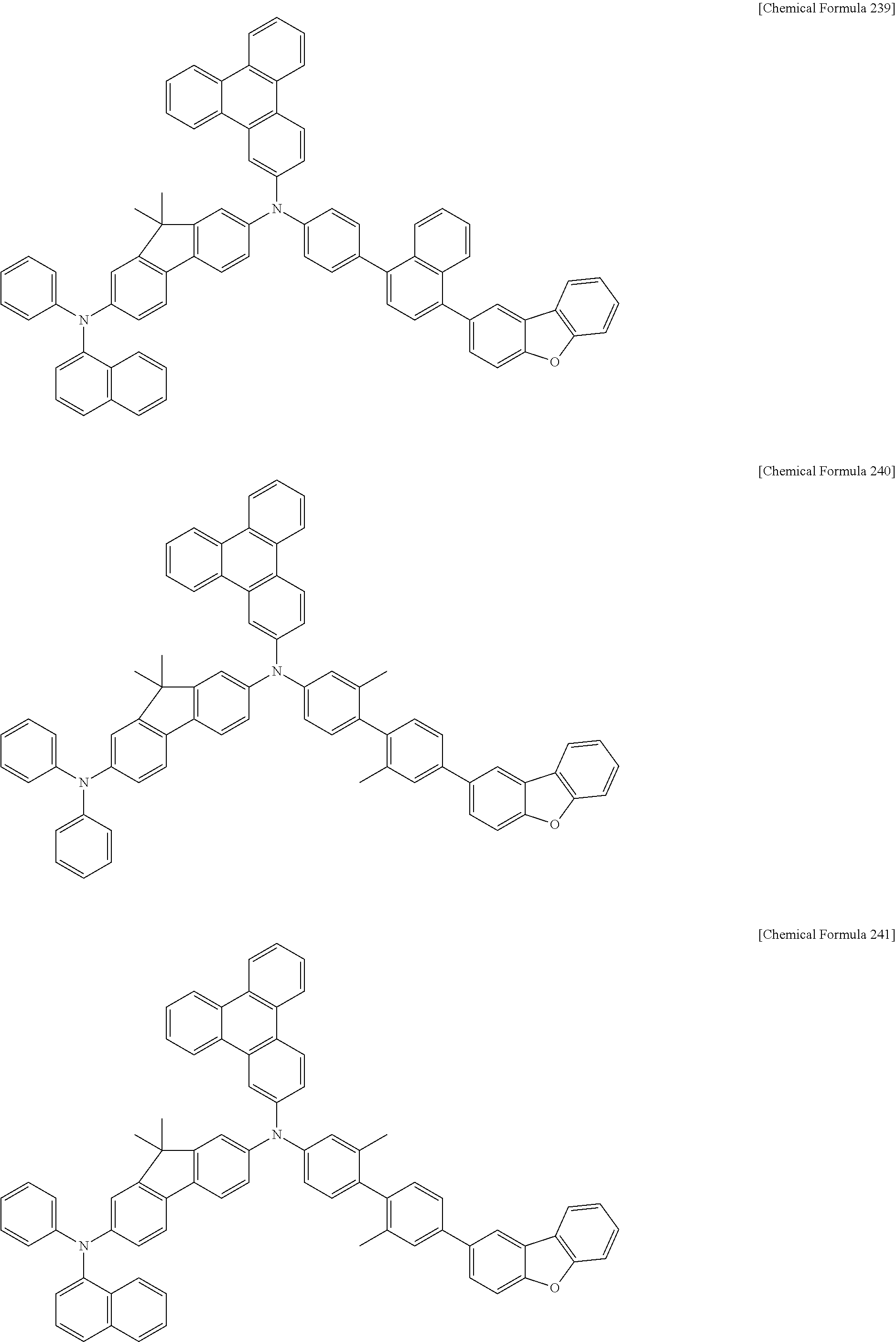 [SMILES: CC1(C)c2cc(N(c3ccc(-c4ccc(-c5ccc6oc7ccccc7c6c5)c5ccccc45)cc3)c3ccc4c5ccccc5c5ccccc5c4c3)ccc2-c2ccc(N(c3ccccc3)c3cccc4ccccc34)cc21 ; Cc1cc(-c2ccc3oc4ccccc4c3c2)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2ccc3oc4ccccc4c3c2)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

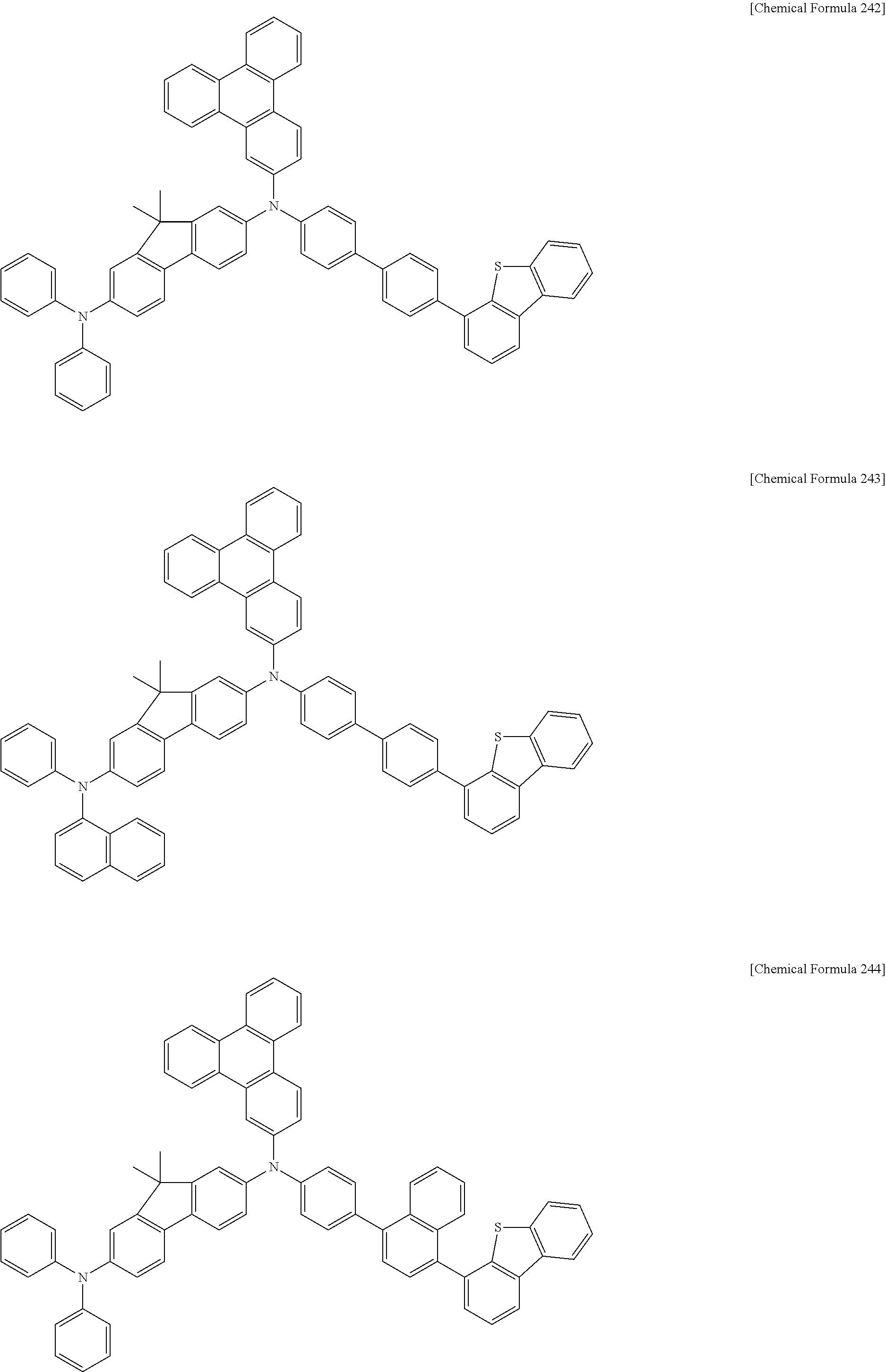 [SMILES: CC1(C)c2cc(N(c3ccccc3)c3ccccc3)ccc2-c2ccc(N(c3ccc(-c4ccc(-c5cccc6c5sc5ccccc56)cc4)cc3)c3ccc4c5ccccc5c5ccccc5c4c3)cc21 ; CC1(C)c2cc(N(c3ccc(-c4ccc(-c5cccc6c5sc5ccccc56)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5cccc6c5sc5ccccc56)c5ccccc45)cc3)c3ccc4c5ccccc5c5ccccc5c4c3)cc21]

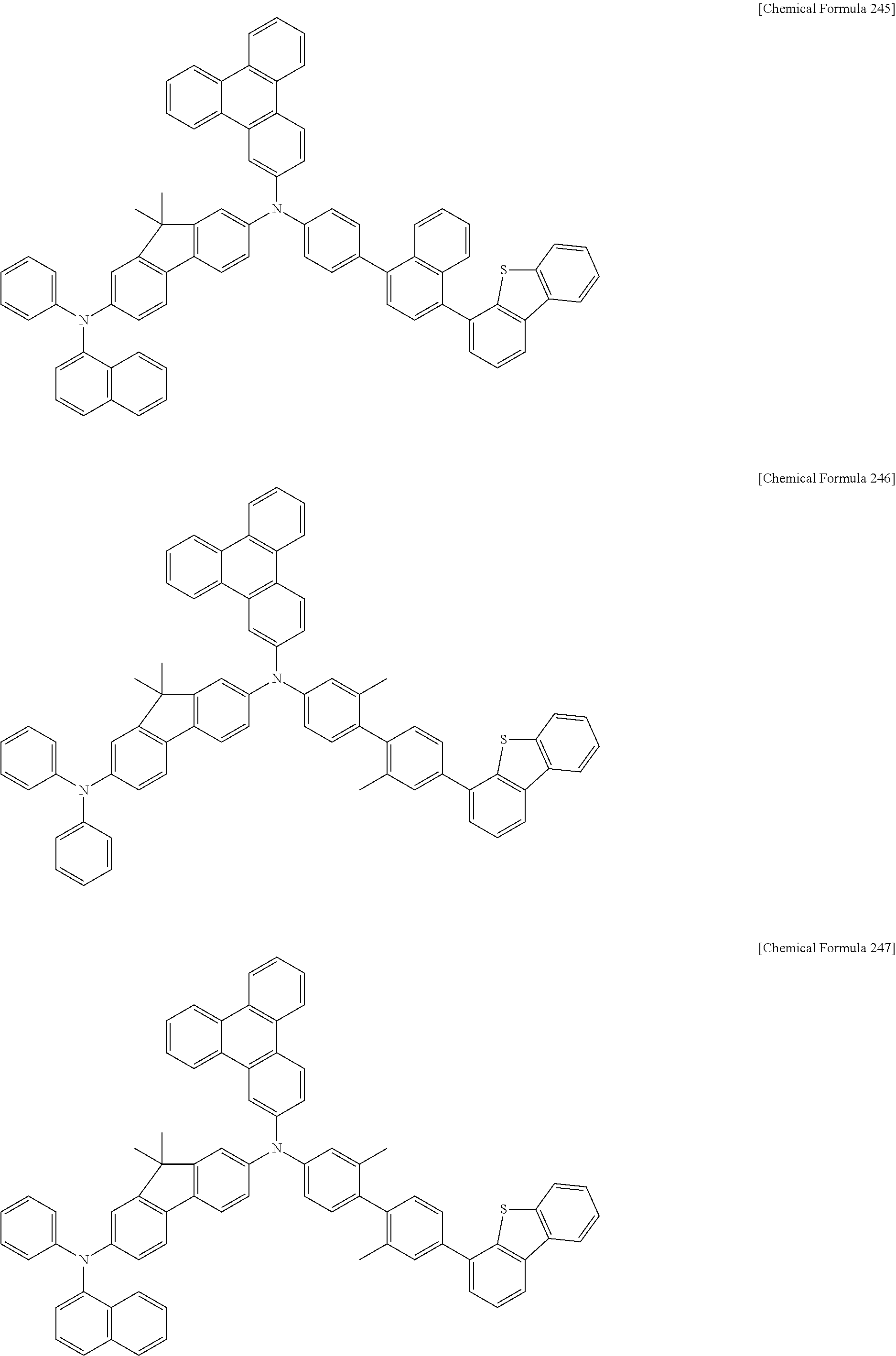 [SMILES: CC1(C)c2cc(N(c3ccc(-c4ccc(-c5cccc6c5sc5ccccc56)c5ccccc45)cc3)c3ccc4c5ccccc5c5ccccc5c4c3)ccc2-c2ccc(N(c3ccccc3)c3cccc4ccccc34)cc21 ; Cc1cc(-c2cccc3c2sc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2cccc3c2sc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

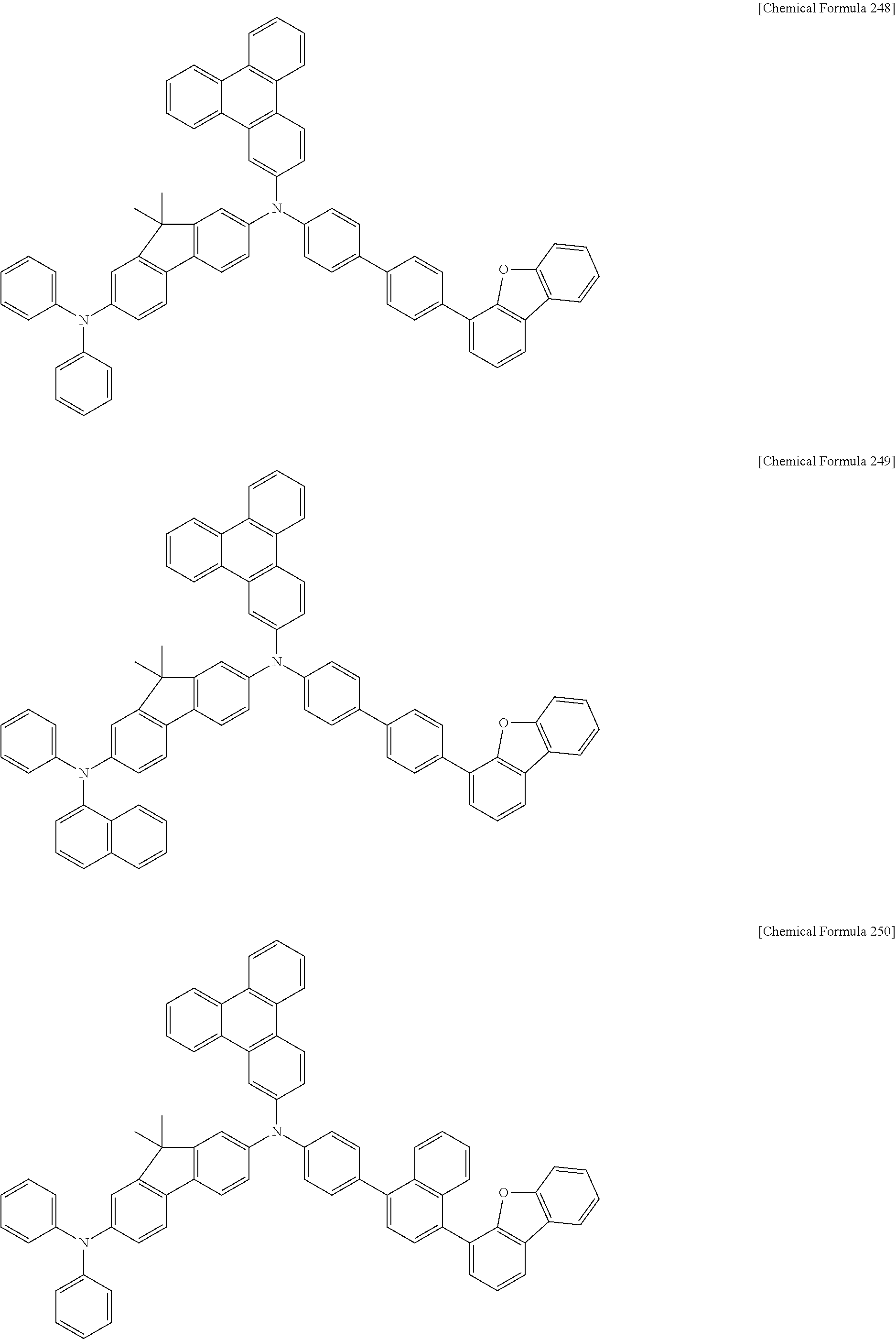 [SMILES: CC1(C)c2cc(N(c3ccccc3)c3ccccc3)ccc2-c2ccc(N(c3ccc(-c4ccc(-c5cccc6c5oc5ccccc56)cc4)cc3)c3ccc4c5ccccc5c5ccccc5c4c3)cc21 ; CC1(C)c2cc(N(c3ccc(-c4ccc(-c5cccc6c5oc5ccccc56)cc4)cc3)c3ccc4c5ccccc5c5ccccc5c4c3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc(-c5cccc6c5oc5ccccc56)c5ccccc45)cc3)c3ccc4c5ccccc5c5ccccc5c4c3)cc21]

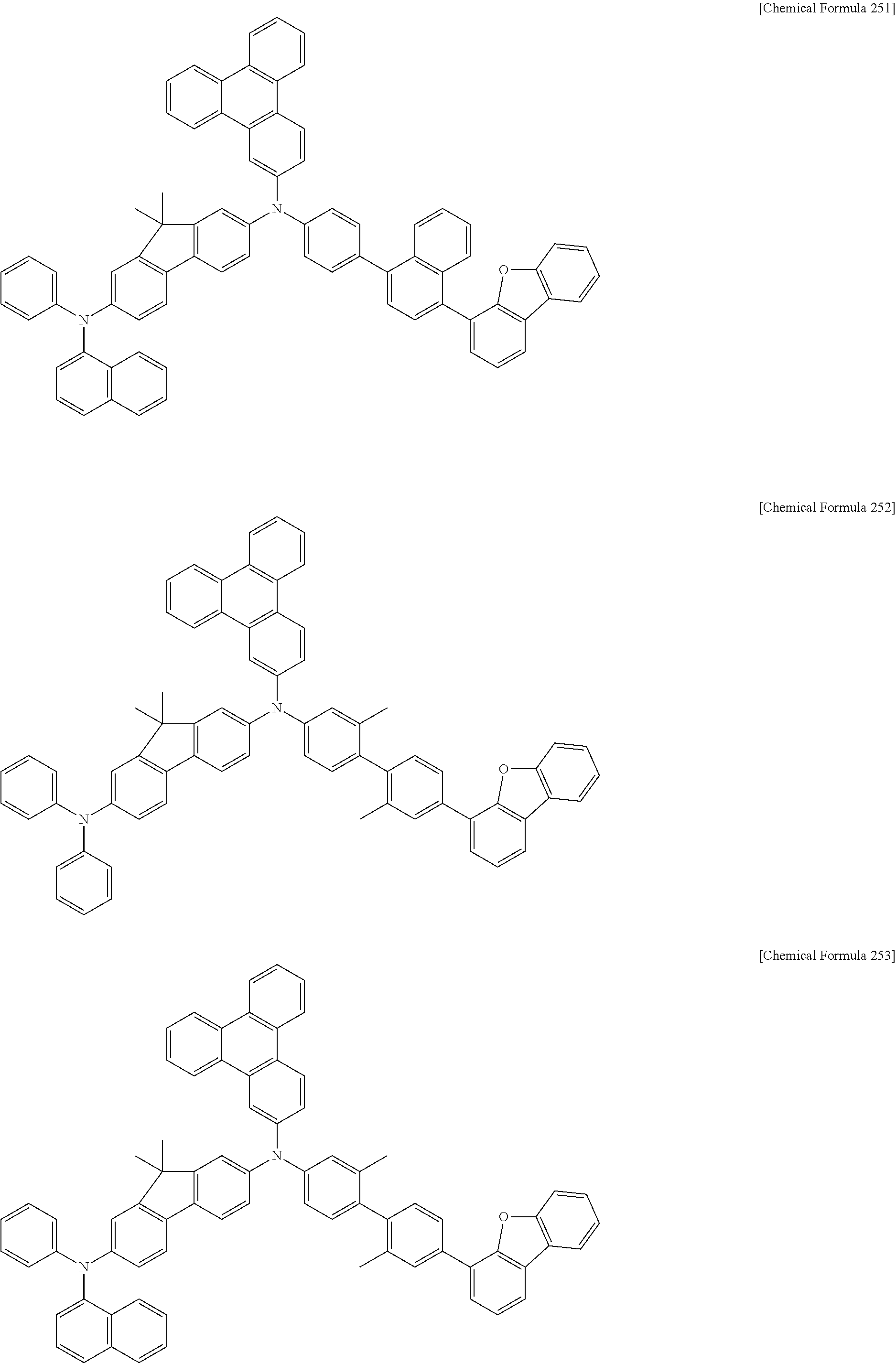 [SMILES: CC1(C)c2cc(N(c3ccc(-c4ccc(-c5cccc6c5oc5ccccc56)c5ccccc45)cc3)c3ccc4c5ccccc5c5ccccc5c4c3)ccc2-c2ccc(N(c3ccccc3)c3cccc4ccccc34)cc21 ; Cc1cc(-c2cccc3c2oc2ccccc23)ccc1-c1ccc(N(c2ccc3c(c2)C(C)(C)c2cc(N(c4ccccc4)c4ccccc4)ccc2-3)c2ccc3c4ccccc4c4ccccc4c3c2)cc1C ; Cc1cc(-c2cccc3c2oc2ccccc23)ccc1-c1ccc(N(c2ccc3c(c2)C(C)(C)c2cc(N(c4ccccc4)c4cccc5ccccc45)ccc2-3)c2ccc3c4ccccc4c4ccccc4c3c2)cc1C]

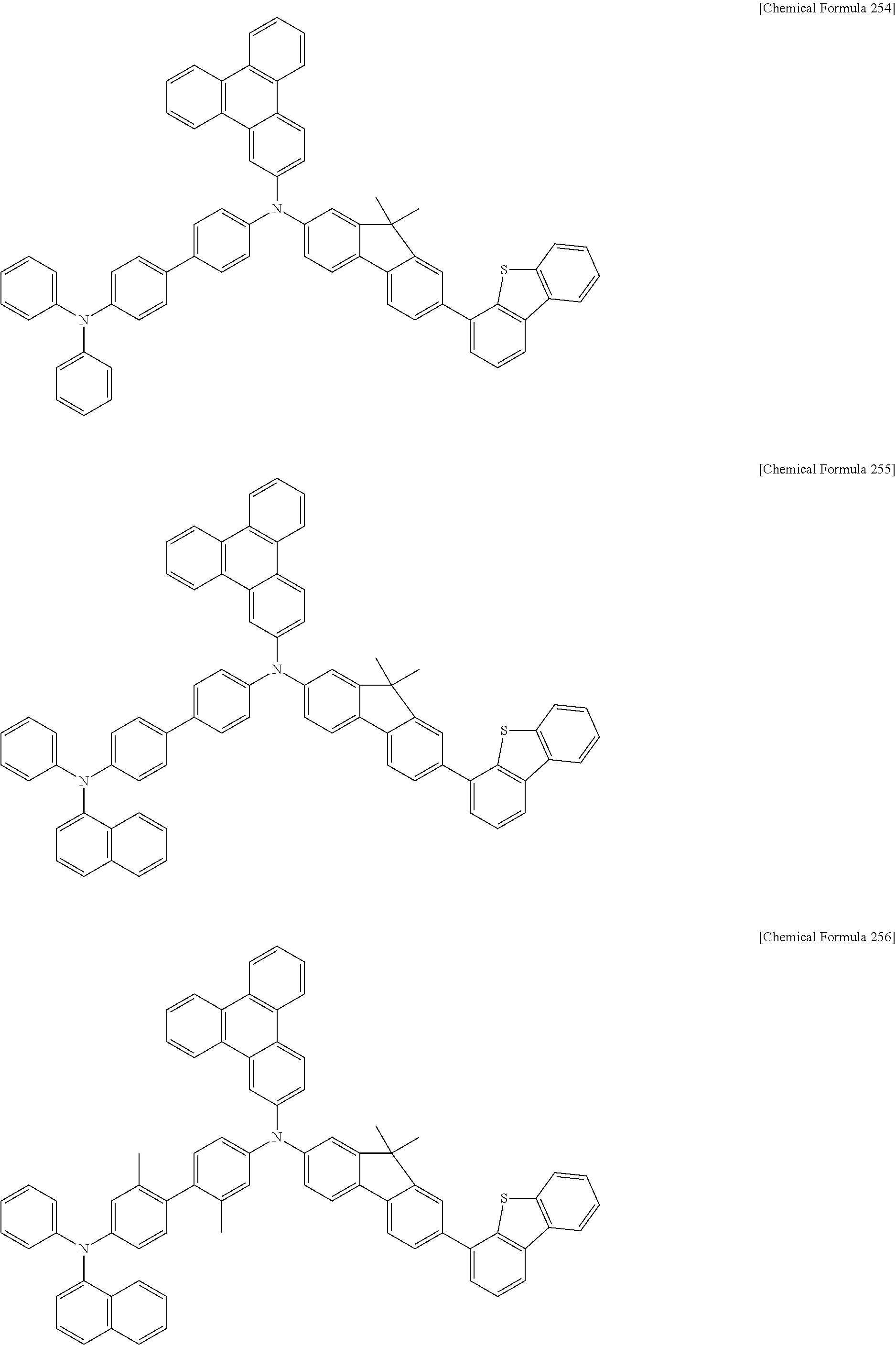 [SMILES: CC1(C)c2cc(-c3cccc4c3sc3ccccc34)ccc2-c2ccc(N(c3ccc(-c4ccc(N(c5ccccc5)c5ccccc5)cc4)cc3)c3ccc4c5ccccc5c5ccccc5c4c3)cc21 ; CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21 ; Cc1cc(N(c2ccc3c(c2)C(C)(C)c2cc(-c4cccc5c4sc4ccccc45)ccc2-3)c2ccc3c4ccccc4c4ccccc4c3c2)ccc1-c1ccc(N(c2ccccc2)c2cccc3ccccc23)cc1C]

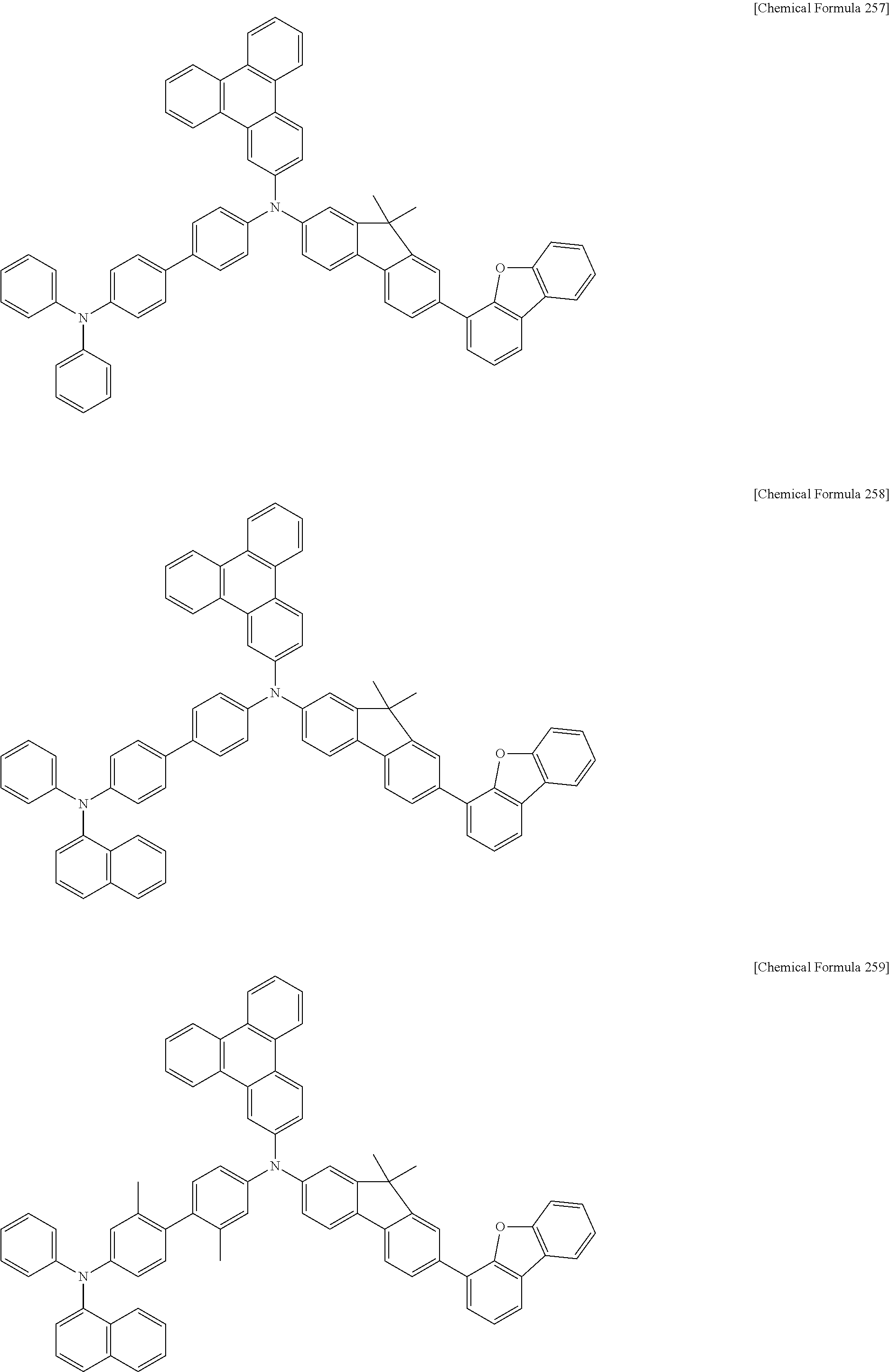 [SMILES: CC1(C)c2cc(-c3cccc4c3oc3ccccc34)ccc2-c2ccc(N(c3ccc(-c4ccc(N(c5ccccc5)c5ccccc5)cc4)cc3)c3ccc4c5ccccc5c5ccccc5c4c3)cc21 ; CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc4c5ccccc5c5ccccc5c4c3)cc21 ; Cc1cc(N(c2ccc3c(c2)C(C)(C)c2cc(-c4cccc5c4oc4ccccc45)ccc2-3)c2ccc3c4ccccc4c4ccccc4c3c2)ccc1-c1ccc(N(c2ccccc2)c2cccc3ccccc23)cc1C]

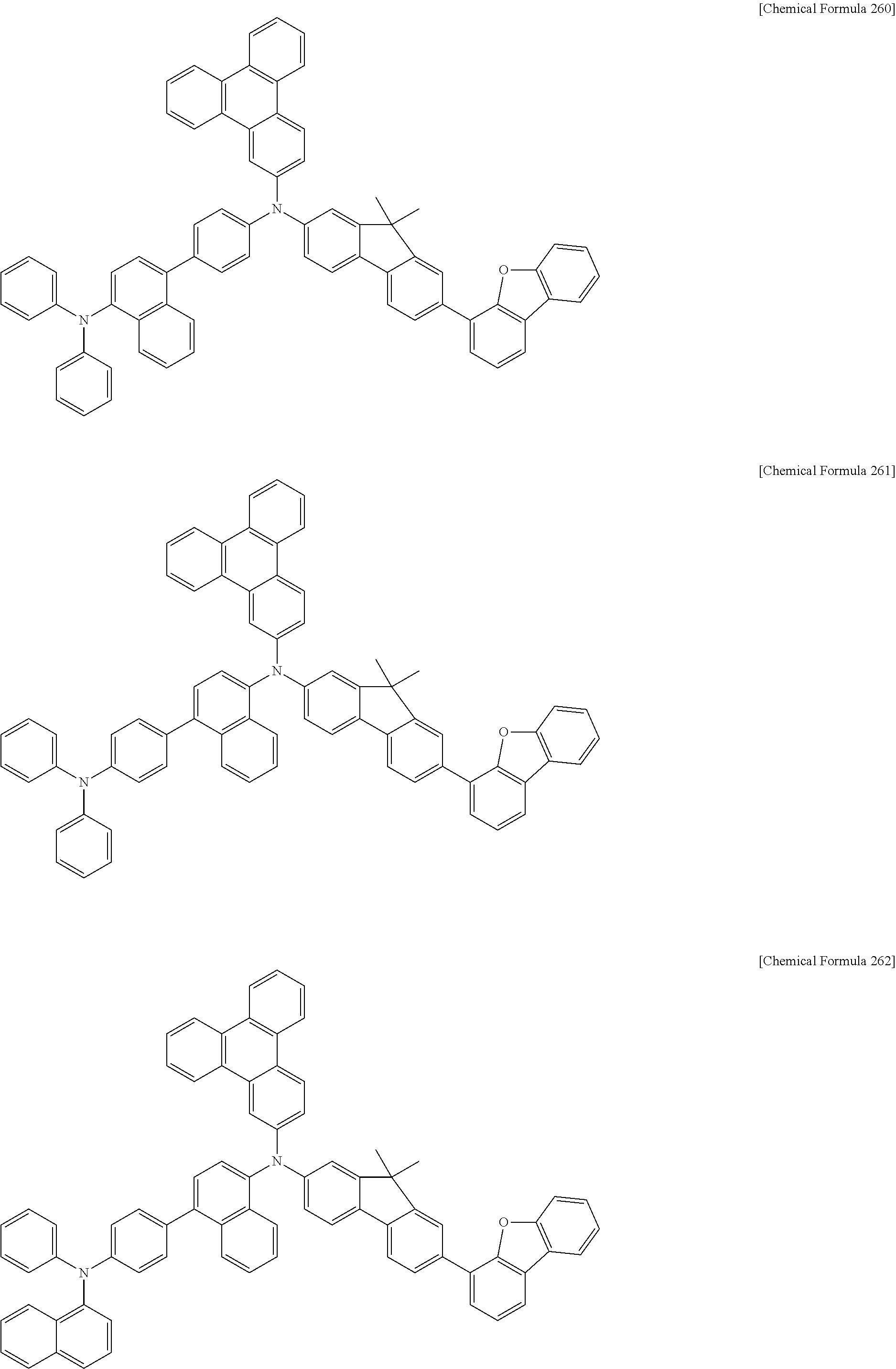 [SMILES: CC1(C)c2cc(-c3cccc4c3oc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21 ; CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21 ; CC1(C)c2cc(-c3cccc4c3oc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21]

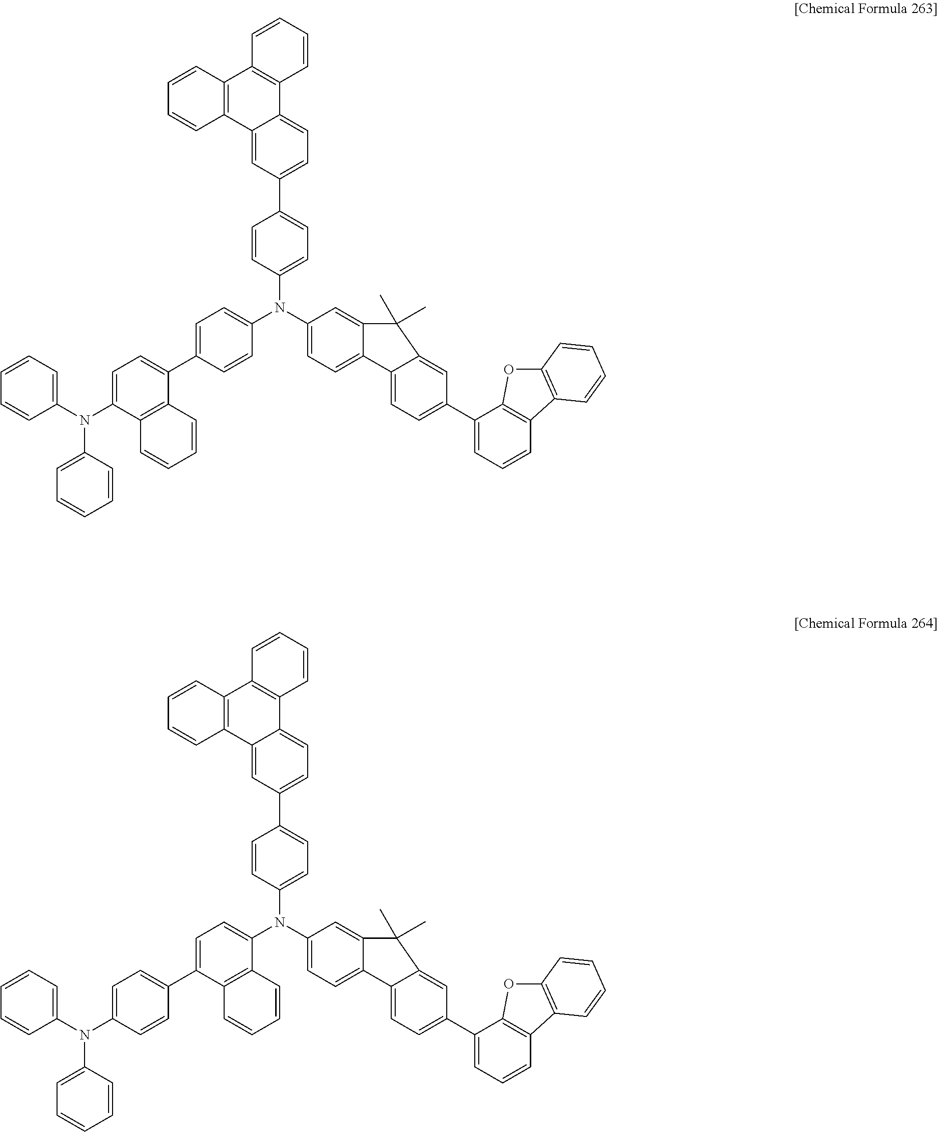 [SMILES: CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21 ; CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21]

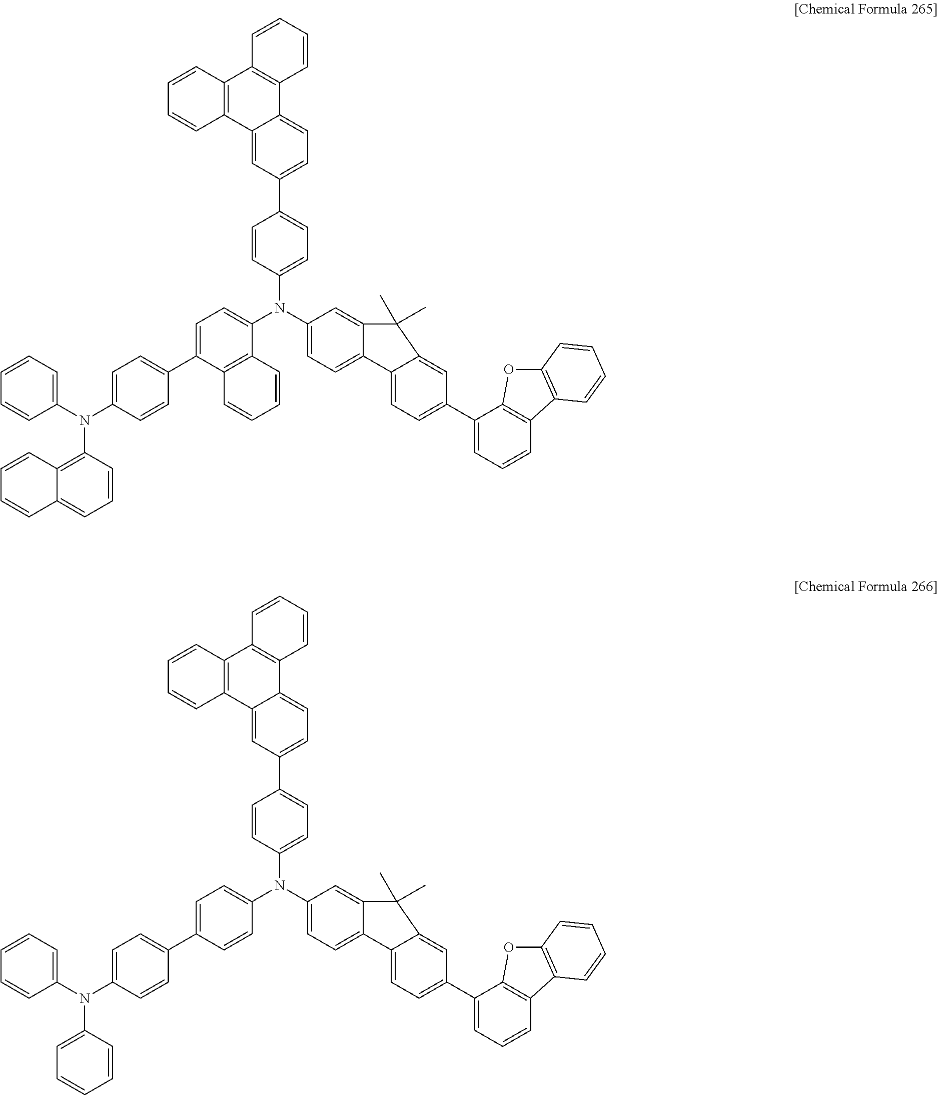 [SMILES: CC1(C)c2cc(-c3cccc4c3oc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21 ; CC1(C)c2cc(-c3cccc4c3oc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21]

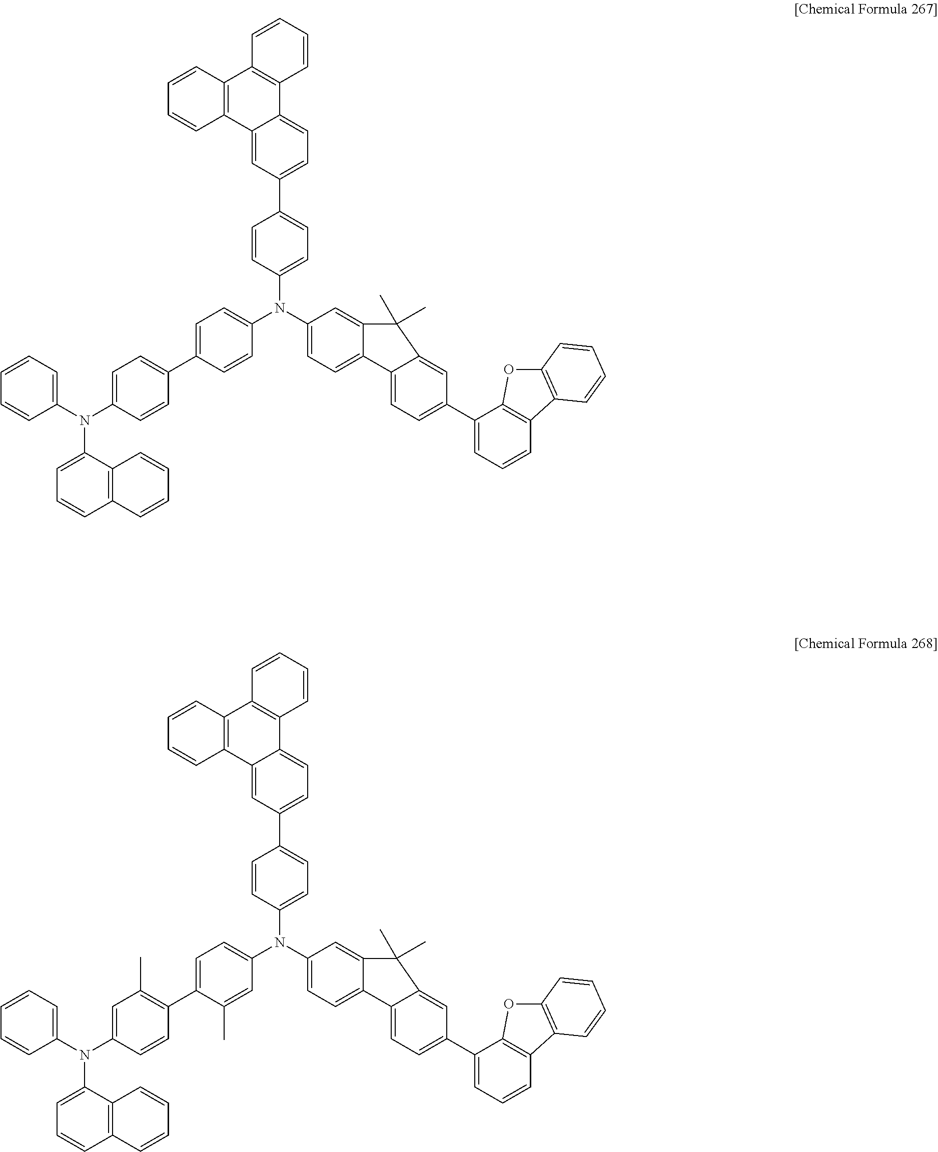 [SMILES: CC1(C)c2cc(-c3cccc4c3oc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21 ; Cc1cc(N(c2ccc(-c3ccc4c5ccccc5c5ccccc5c4c3)cc2)c2ccc3c(c2)C(C)(C)c2cc(-c4cccc5c4oc4ccccc45)ccc2-3)ccc1-c1ccc(N(c2ccccc2)c2cccc3ccccc23)cc1C]

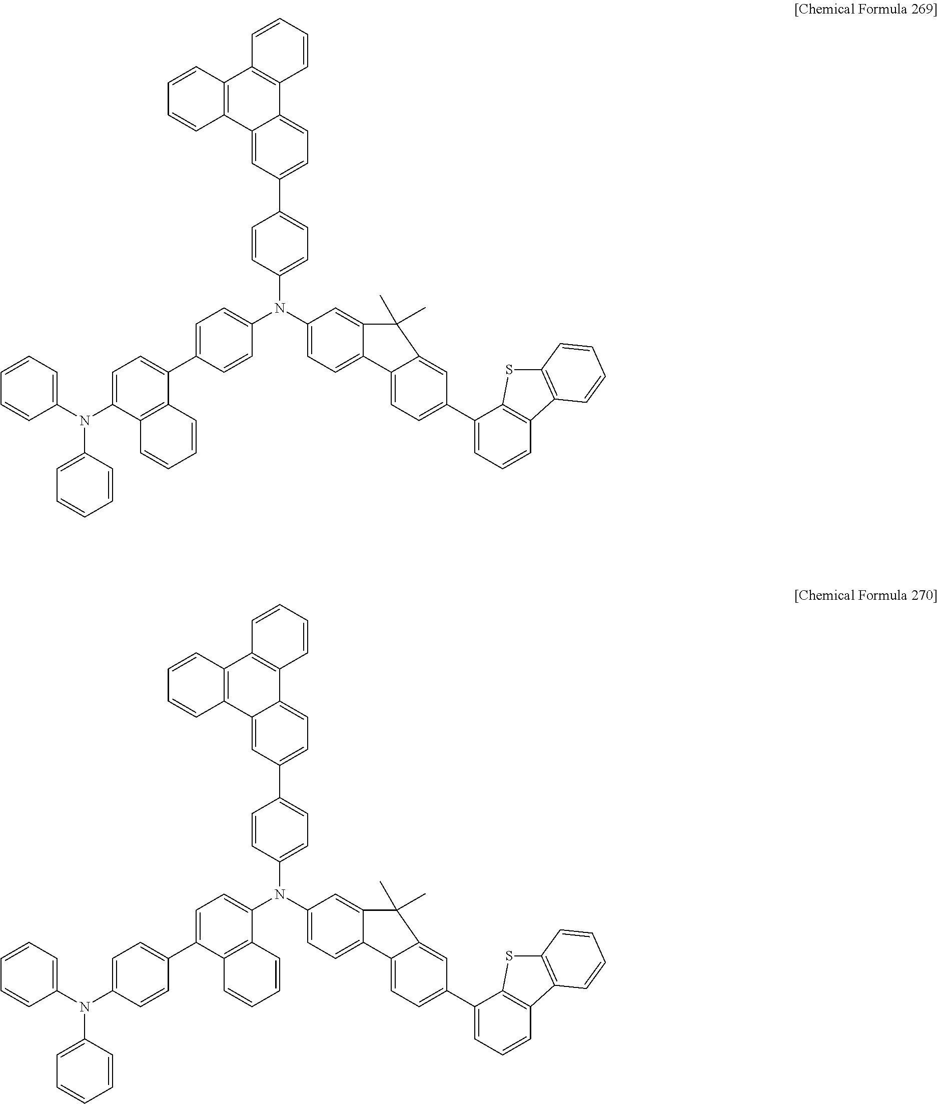 [SMILES: CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)c5ccccc45)cc3)cc21 ; CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5ccccc5)cc4)c4ccccc34)cc21]

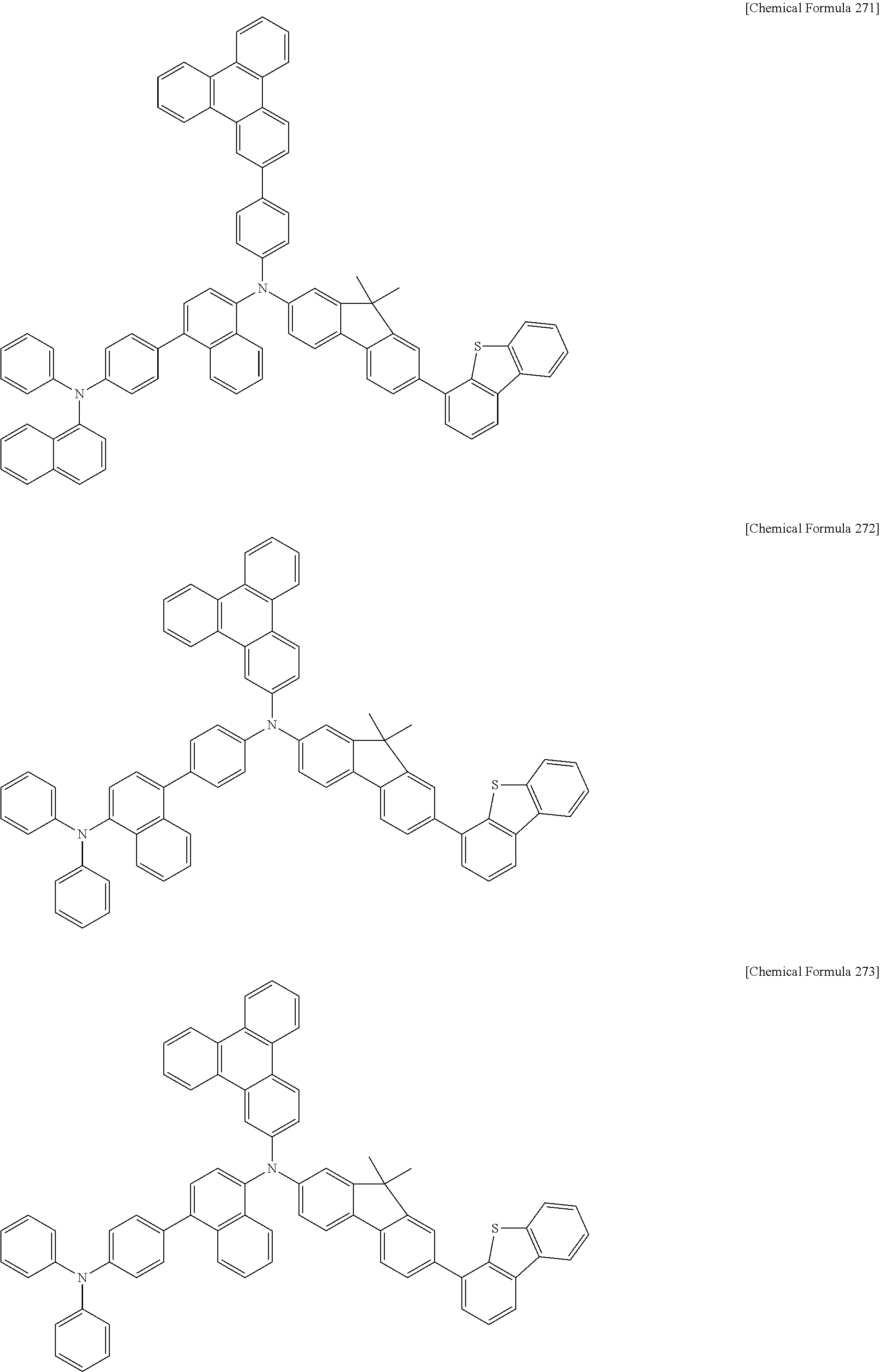 [SMILES: CC1(C)c2cc(-c3cccc4c3sc3ccccc34)ccc2-c2ccc(N(c3ccc(-c4ccc5c6ccccc6c6ccccc6c5c4)cc3)c3ccc(-c4ccc(N(c5ccccc5)c5cccc6ccccc56)cc4)c4ccccc34)cc21 ; CC1(C)c2cc(-c3cccc4c3sc3ccccc34)ccc2-c2ccc(N(c3ccc(-c4ccc(N(c5ccccc5)c5ccccc5)c5ccccc45)cc3)c3ccc4c5ccccc5c5ccccc5c4c3)cc21 ; CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5ccccc5)cc4)c4ccccc34)cc21]

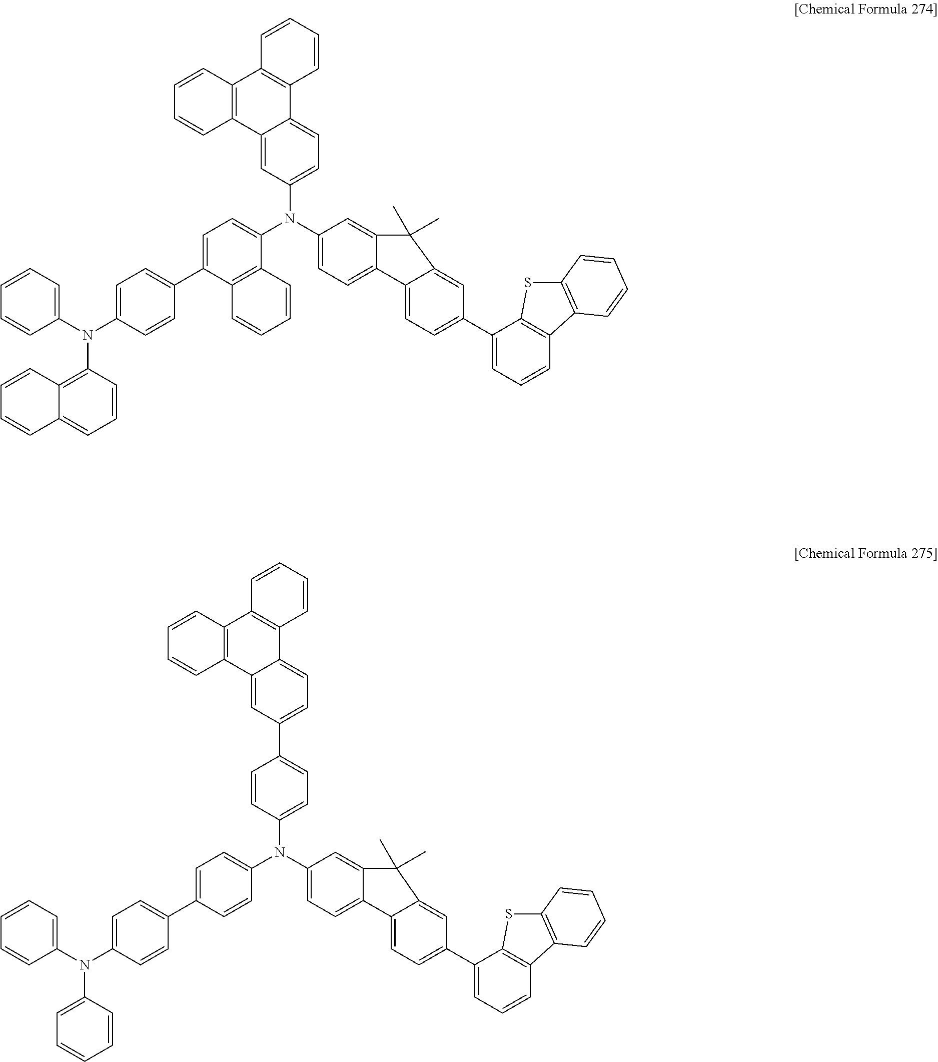 [SMILES: CC1(C)c2cc(-c3cccc4c3sc3ccccc34)ccc2-c2ccc(N(c3ccc4c5ccccc5c5ccccc5c4c3)c3ccc(-c4ccc(N(c5ccccc5)c5cccc6ccccc56)cc4)c4ccccc34)cc21 ; CC1(C)c2cc(-c3cccc4c3sc3ccccc34)ccc2-c2ccc(N(c3ccc(-c4ccc(N(c5ccccc5)c5ccccc5)cc4)cc3)c3ccc(-c4ccc5c6ccccc6c6ccccc6c5c4)cc3)cc21]

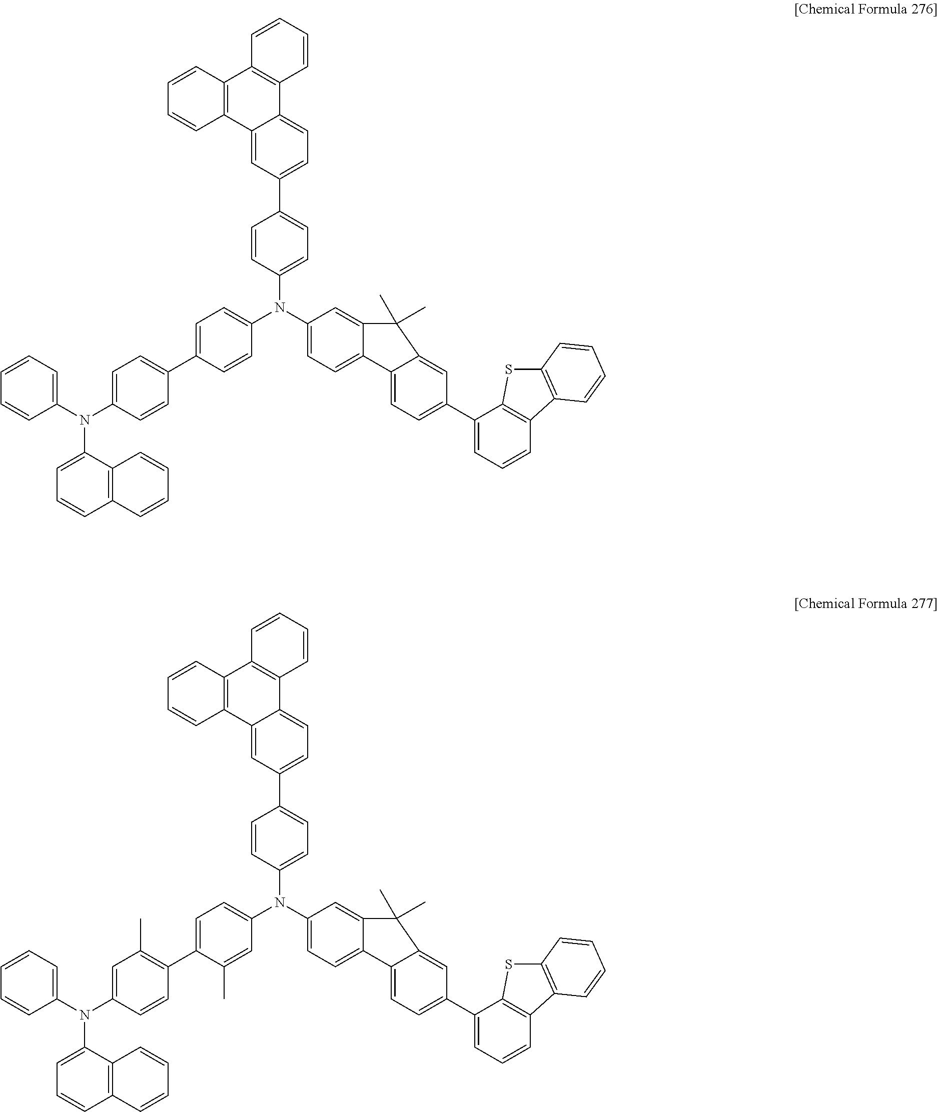 [SMILES: CC1(C)c2cc(-c3cccc4c3sc3ccccc34)ccc2-c2ccc(N(c3ccc(-c4ccc(N(c5ccccc5)c5cccc6ccccc56)cc4)cc3)c3ccc(-c4ccc5c6ccccc6c6ccccc6c5c4)cc3)cc21 ; Cc1cc(N(c2ccc(-c3ccc4c5ccccc5c5ccccc5c4c3)cc2)c2ccc3c(c2)C(C)(C)c2cc(-c4cccc5c4sc4ccccc45)ccc2-3)ccc1-c1ccc(N(c2ccccc2)c2cccc3ccccc23)cc1C]

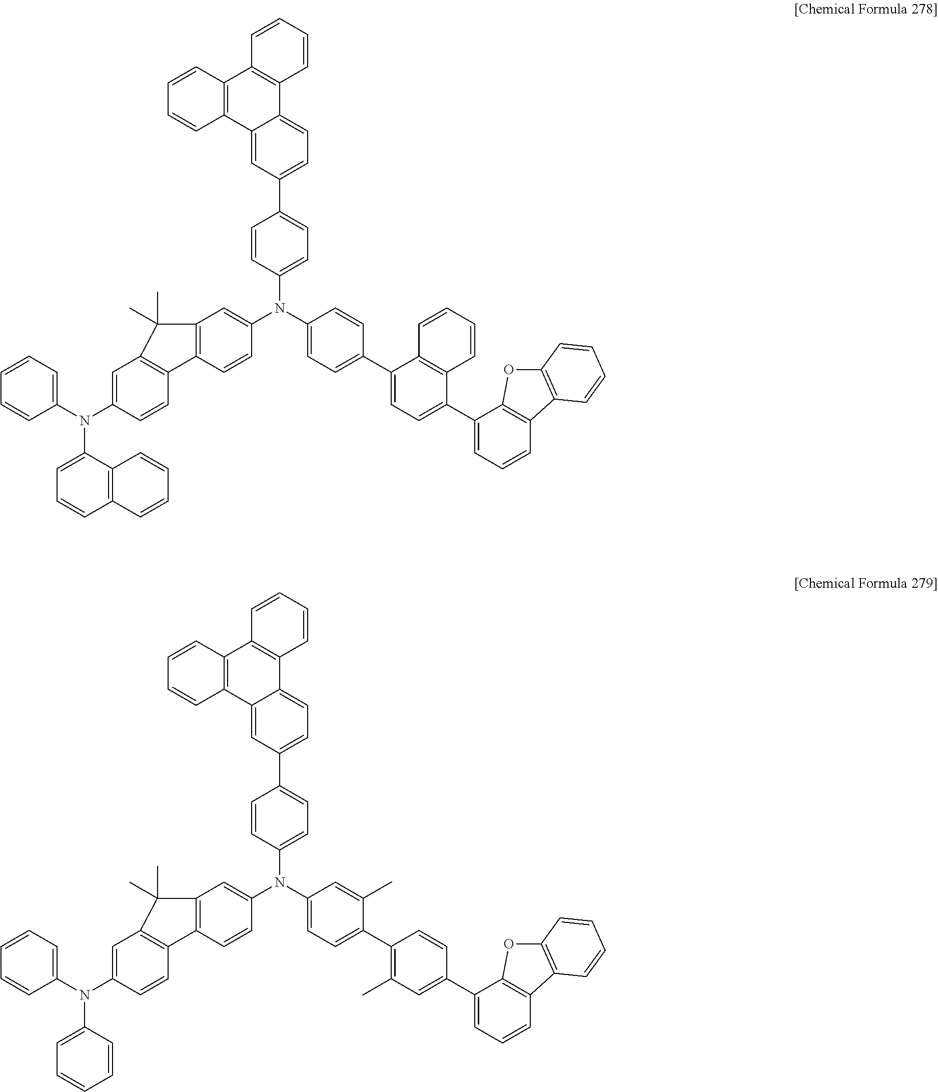 [SMILES: CC1(C)c2cc(N(c3ccc(-c4ccc5c6ccccc6c6ccccc6c5c4)cc3)c3ccc(-c4ccc(-c5cccc6c5oc5ccccc56)c5ccccc45)cc3)ccc2-c2ccc(N(c3ccccc3)c3cccc4ccccc34)cc21 ; Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

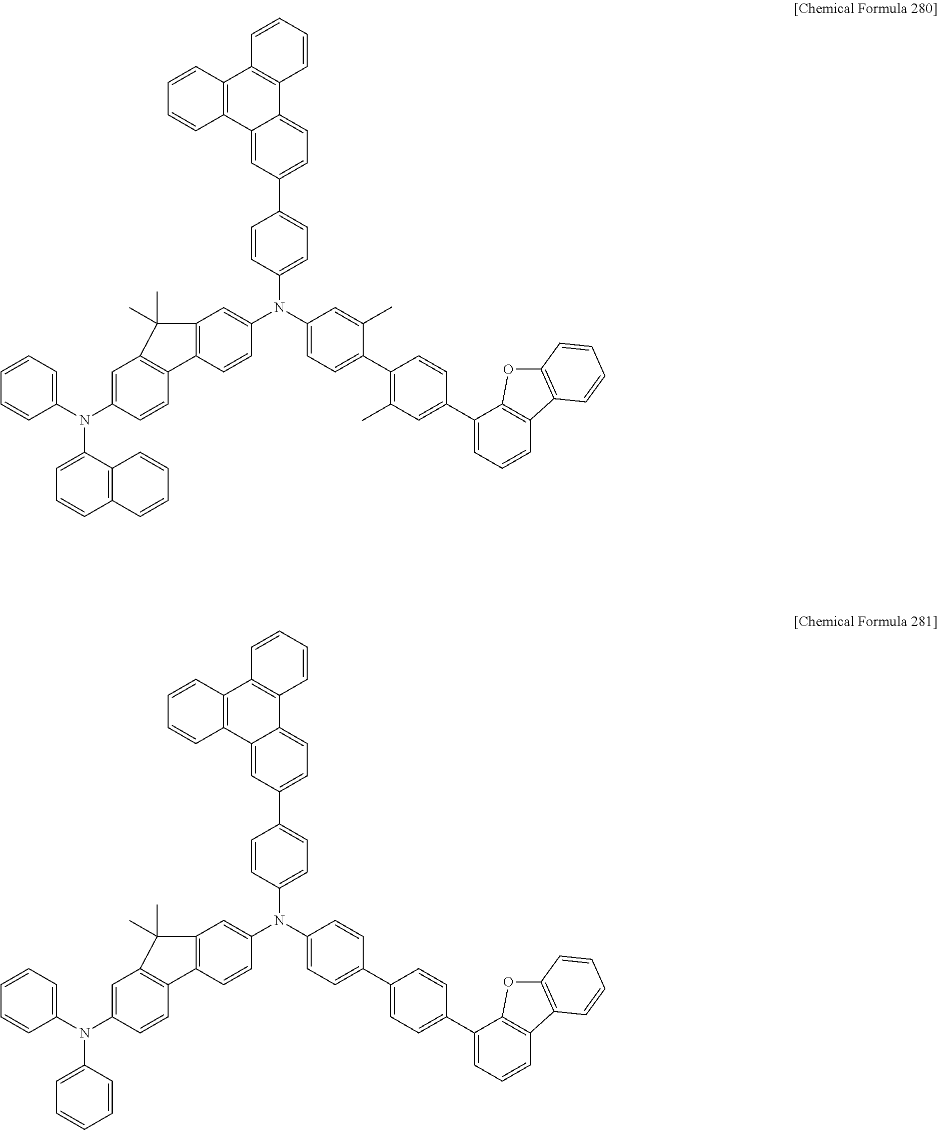 [SMILES: Cc1cc(-c2cccc3c2oc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5cccc6c5oc5ccccc56)cc4)cc3)c3ccc(-c4ccc5c6ccccc6c6ccccc6c5c4)cc3)cc21]

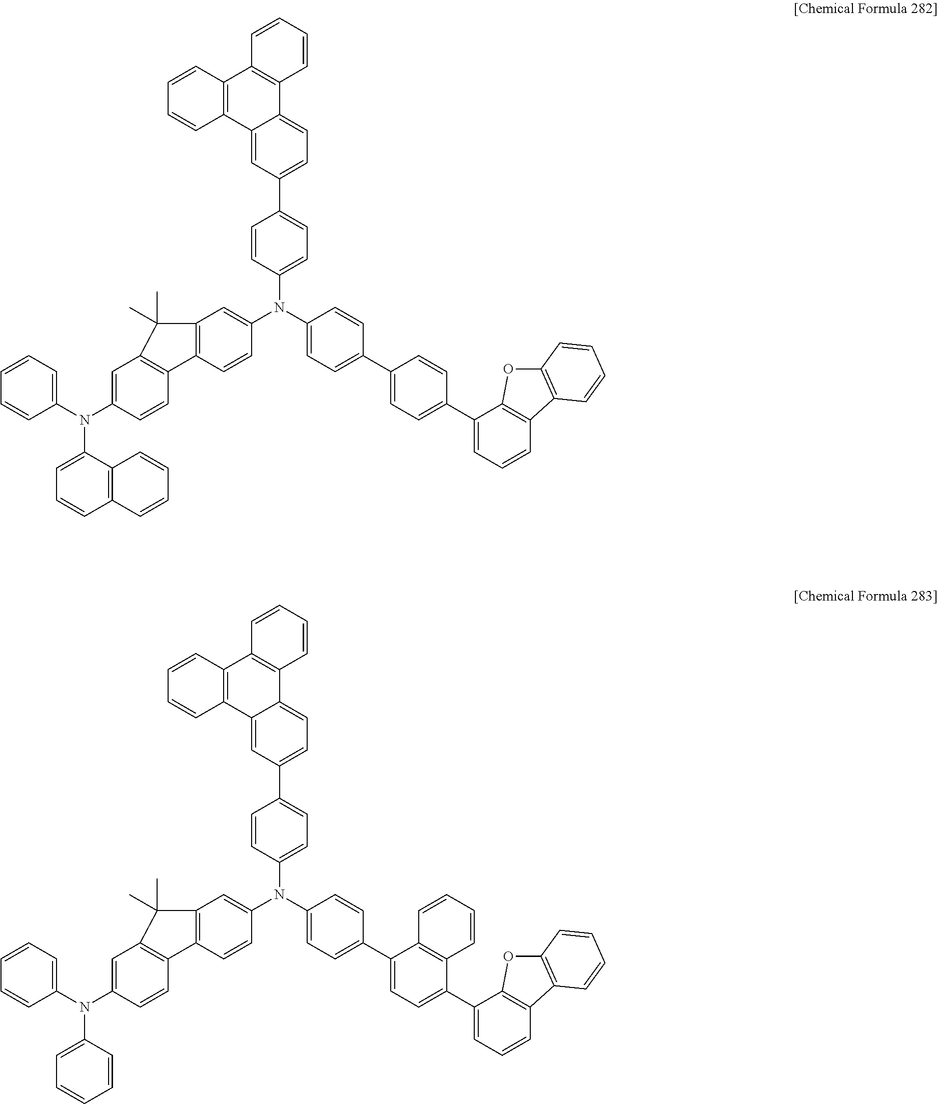 [SMILES: CC1(C)c2cc(N(c3ccc(-c4ccc(-c5cccc6c5oc5ccccc56)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5oc5ccccc56)c5ccccc45)cc3)cc21]

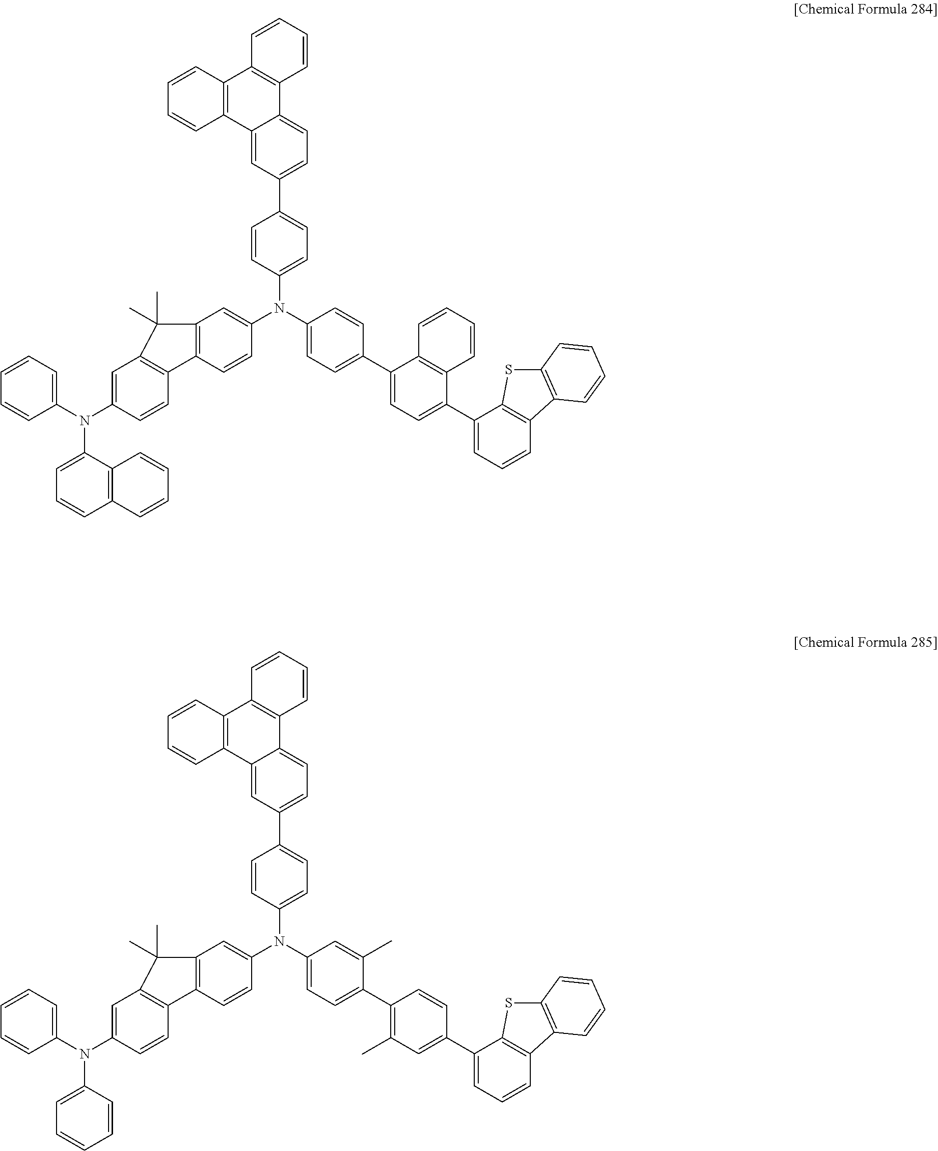 [SMILES: CC1(C)c2cc(N(c3ccc(-c4ccc5c6ccccc6c6ccccc6c5c4)cc3)c3ccc(-c4ccc(-c5cccc6c5sc5ccccc56)c5ccccc45)cc3)ccc2-c2ccc(N(c3ccccc3)c3cccc4ccccc34)cc21 ; Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

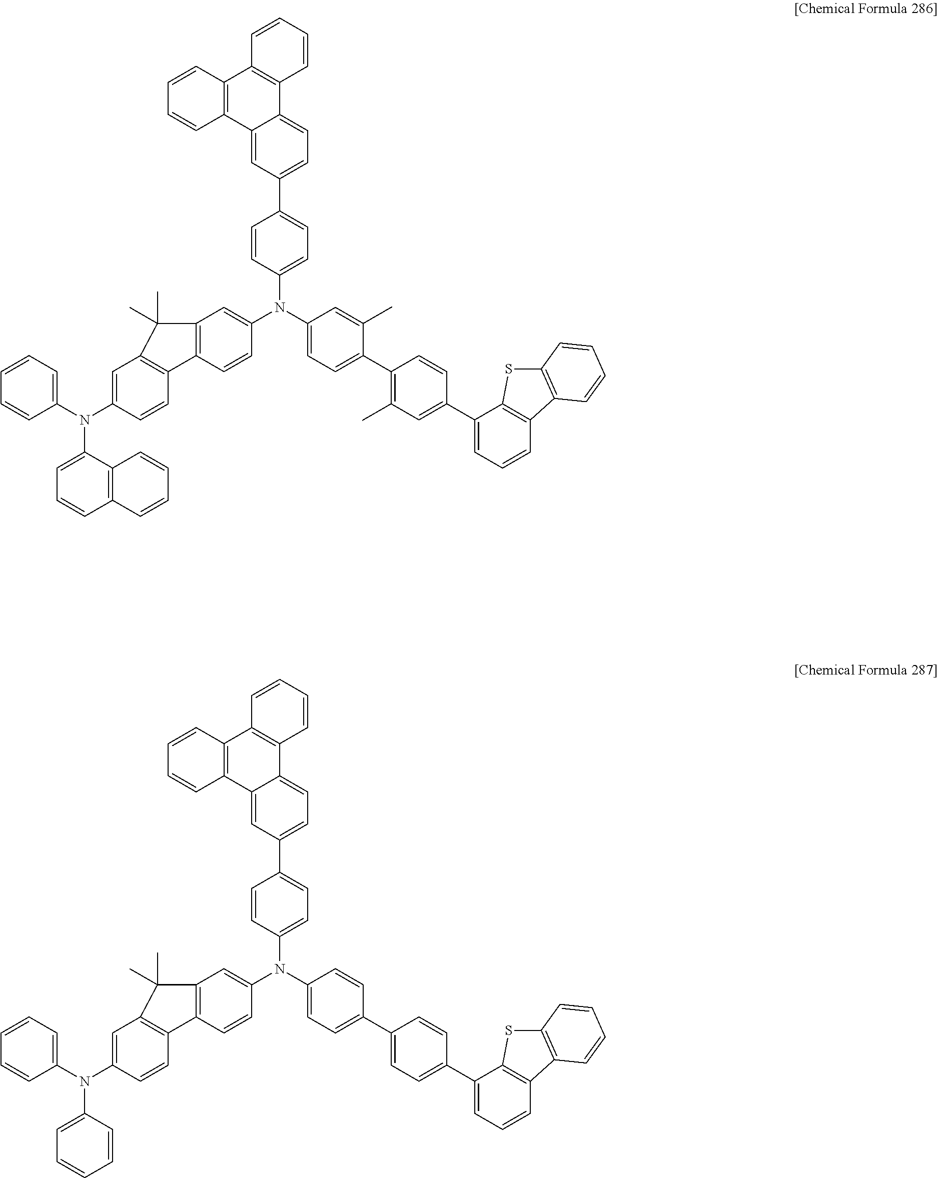 [SMILES: Cc1cc(-c2cccc3c2sc2ccccc23)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5cccc6c5sc5ccccc56)cc4)cc3)c3ccc(-c4ccc5c6ccccc6c6ccccc6c5c4)cc3)cc21]

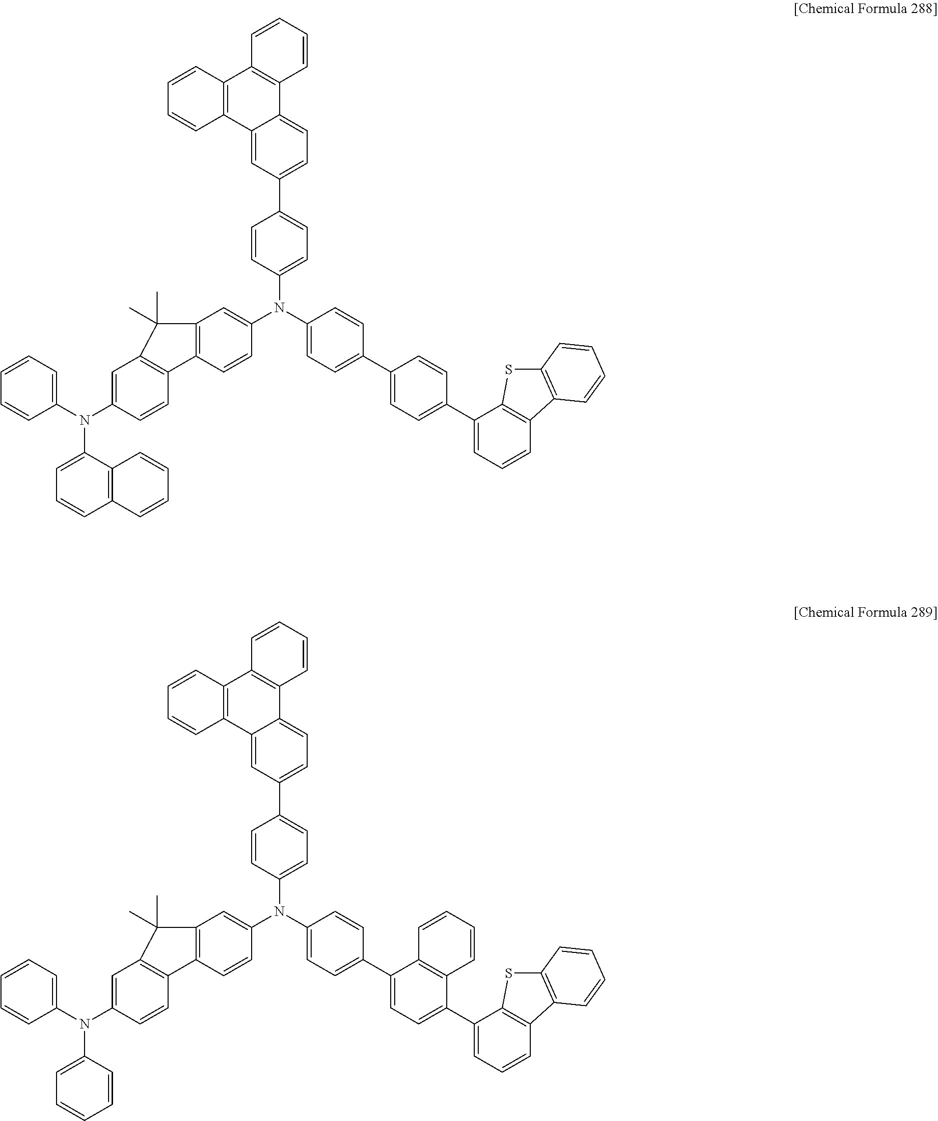 [SMILES: CC1(C)c2cc(N(c3ccc(-c4ccc(-c5cccc6c5sc5ccccc56)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5cccc6c5sc5ccccc56)c5ccccc45)cc3)cc21]

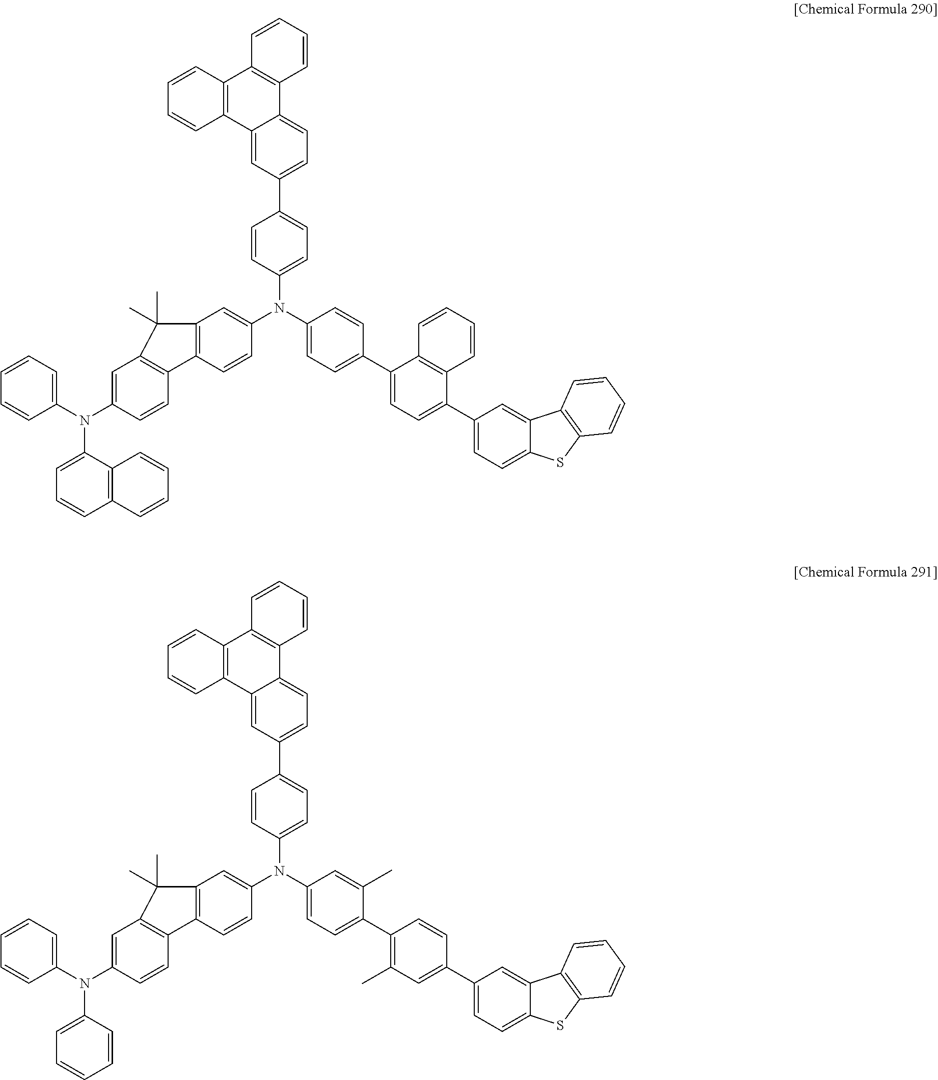 [SMILES: CC1(C)c2cc(N(c3ccc(-c4ccc5c6ccccc6c6ccccc6c5c4)cc3)c3ccc(-c4ccc(-c5ccc6sc7ccccc7c6c5)c5ccccc45)cc3)ccc2-c2ccc(N(c3ccccc3)c3cccc4ccccc34)cc21 ; Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

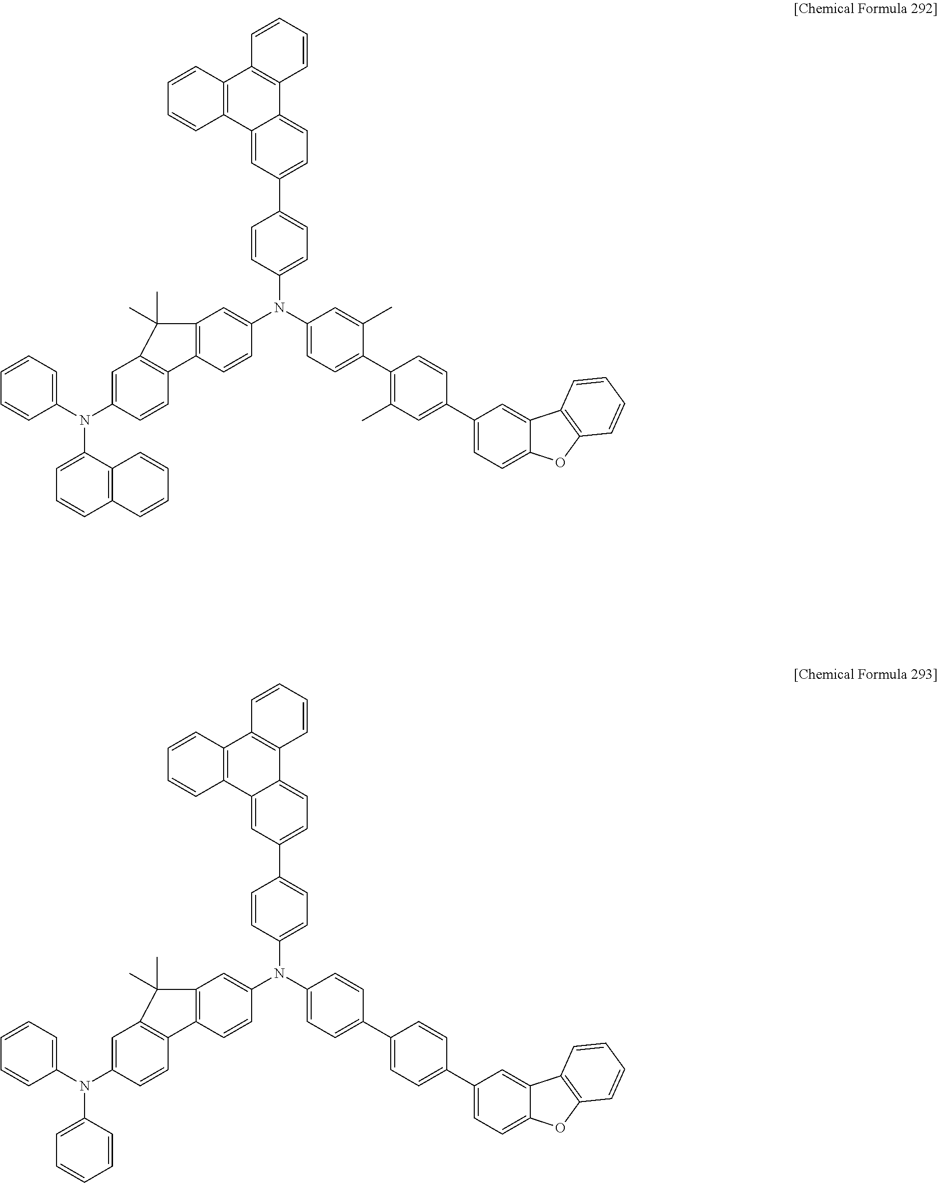 [SMILES: Cc1cc(-c2ccc3oc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5ccc6oc7ccccc7c6c5)cc4)cc3)c3ccc(-c4ccc5c6ccccc6c6ccccc6c5c4)cc3)cc21]

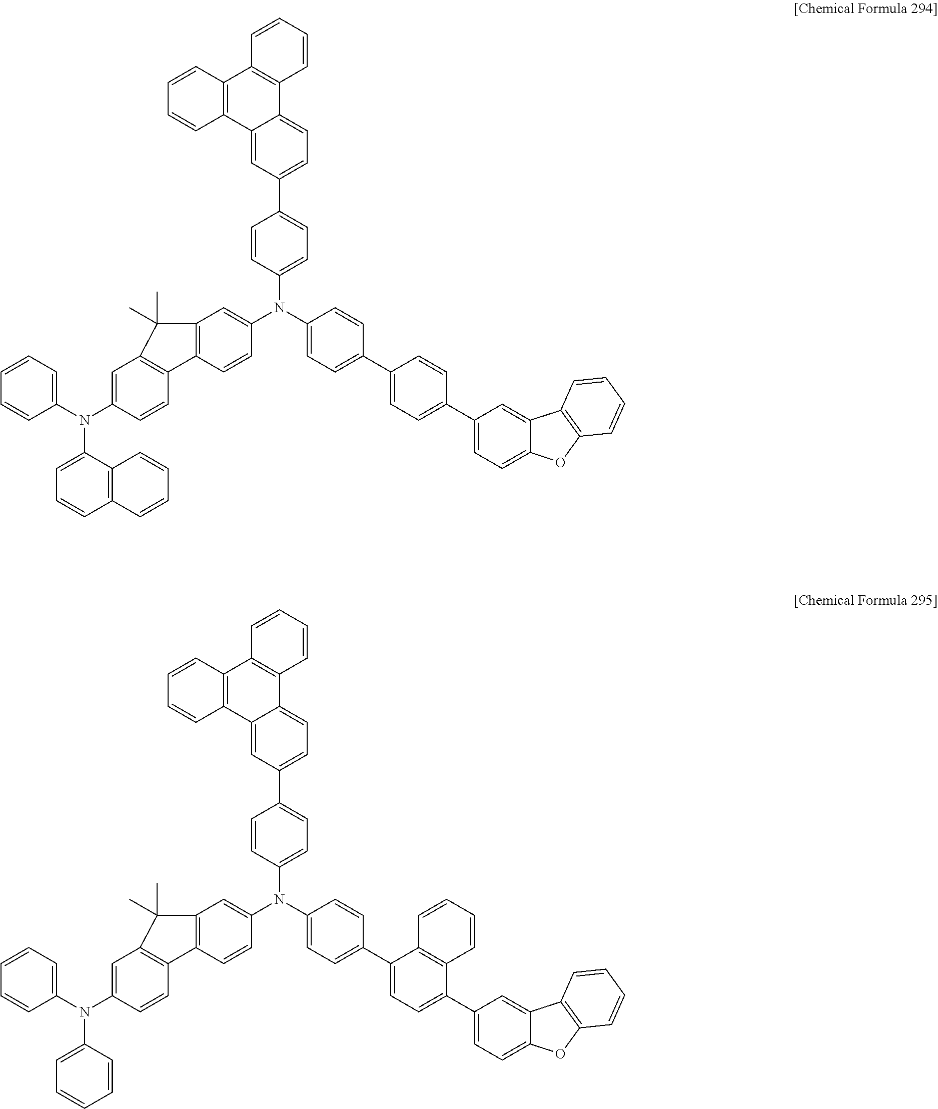 [SMILES: CC1(C)c2cc(N(c3ccc(-c4ccc(-c5ccc6oc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6oc7ccccc7c6c5)c5ccccc45)cc3)cc21]

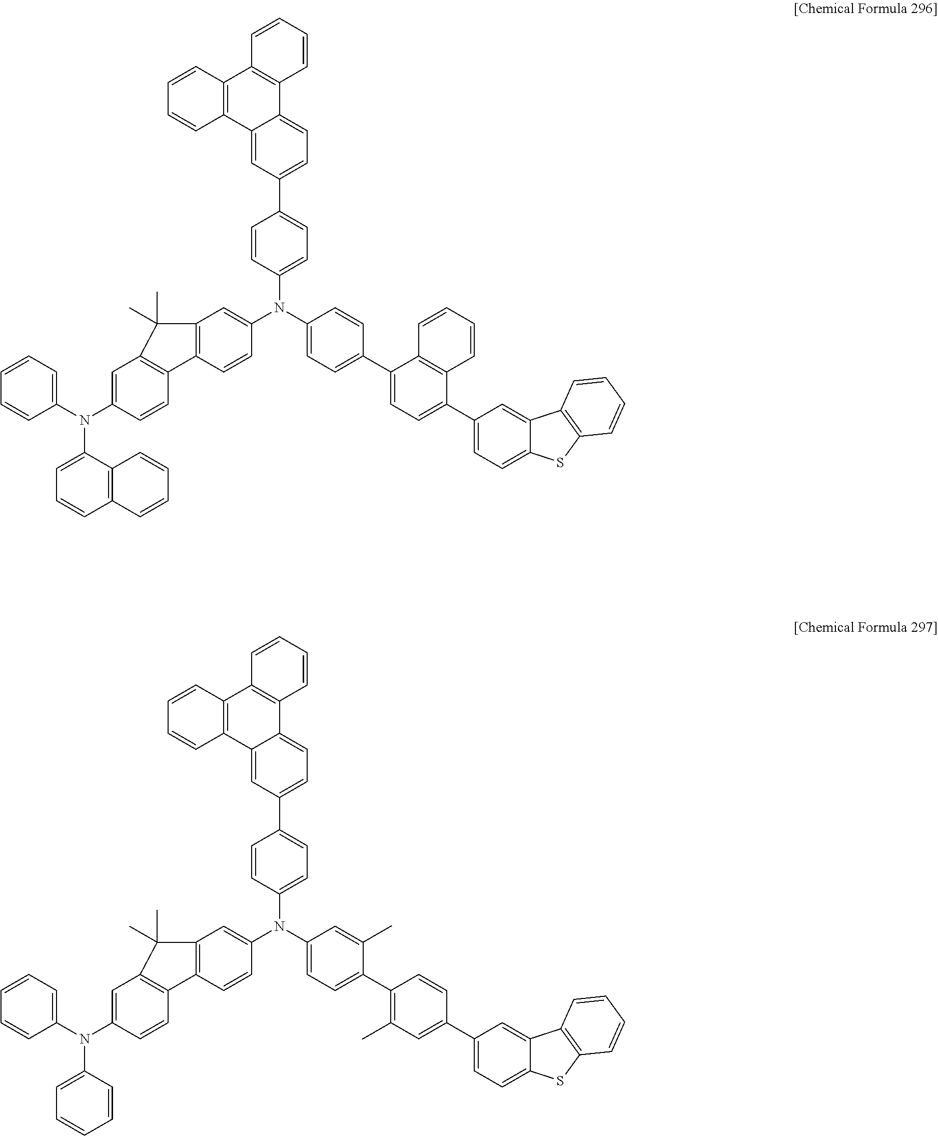 [SMILES: CC1(C)c2cc(N(c3ccc(-c4ccc5c6ccccc6c6ccccc6c5c4)cc3)c3ccc(-c4ccc(-c5ccc6sc7ccccc7c6c5)c5ccccc45)cc3)ccc2-c2ccc(N(c3ccccc3)c3cccc4ccccc34)cc21 ; Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C]

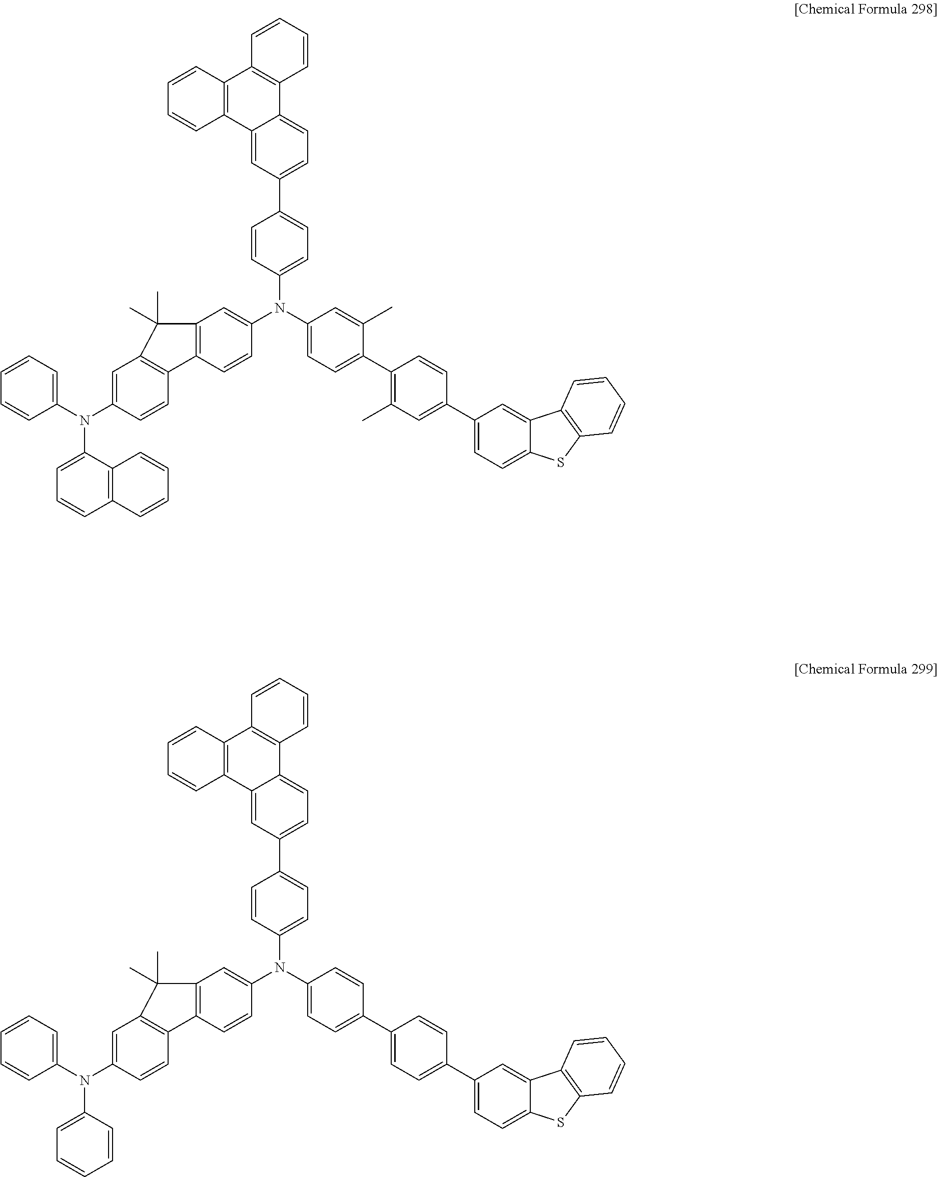 [SMILES: Cc1cc(-c2ccc3sc4ccccc4c3c2)ccc1-c1ccc(N(c2ccc(-c3ccc4c5ccccc5c5ccccc5c4c3)cc2)c2ccc3c(c2)C(C)(C)c2cc(N(c4ccccc4)c4cccc5ccccc45)ccc2-3)cc1C ; CC1(C)c2cc(N(c3ccccc3)c3ccccc3)ccc2-c2ccc(N(c3ccc(-c4ccc(-c5ccc6sc7ccccc7c6c5)cc4)cc3)c3ccc(-c4ccc5c6ccccc6c6ccccc6c5c4)cc3)cc21]

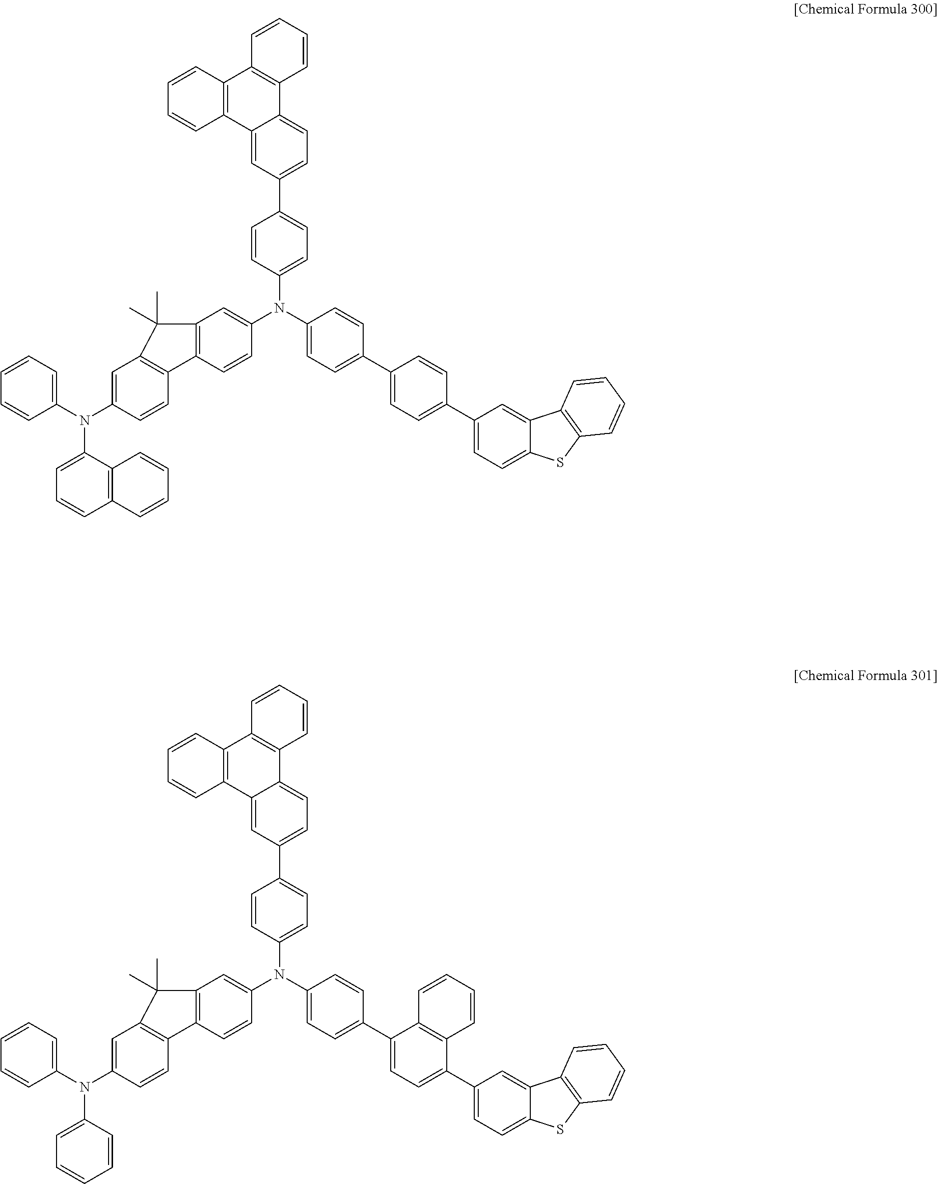 [SMILES: CC1(C)c2cc(N(c3ccc(-c4ccc(-c5ccc6sc7ccccc7c6c5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6sc7ccccc7c6c5)c5ccccc45)cc3)cc21]

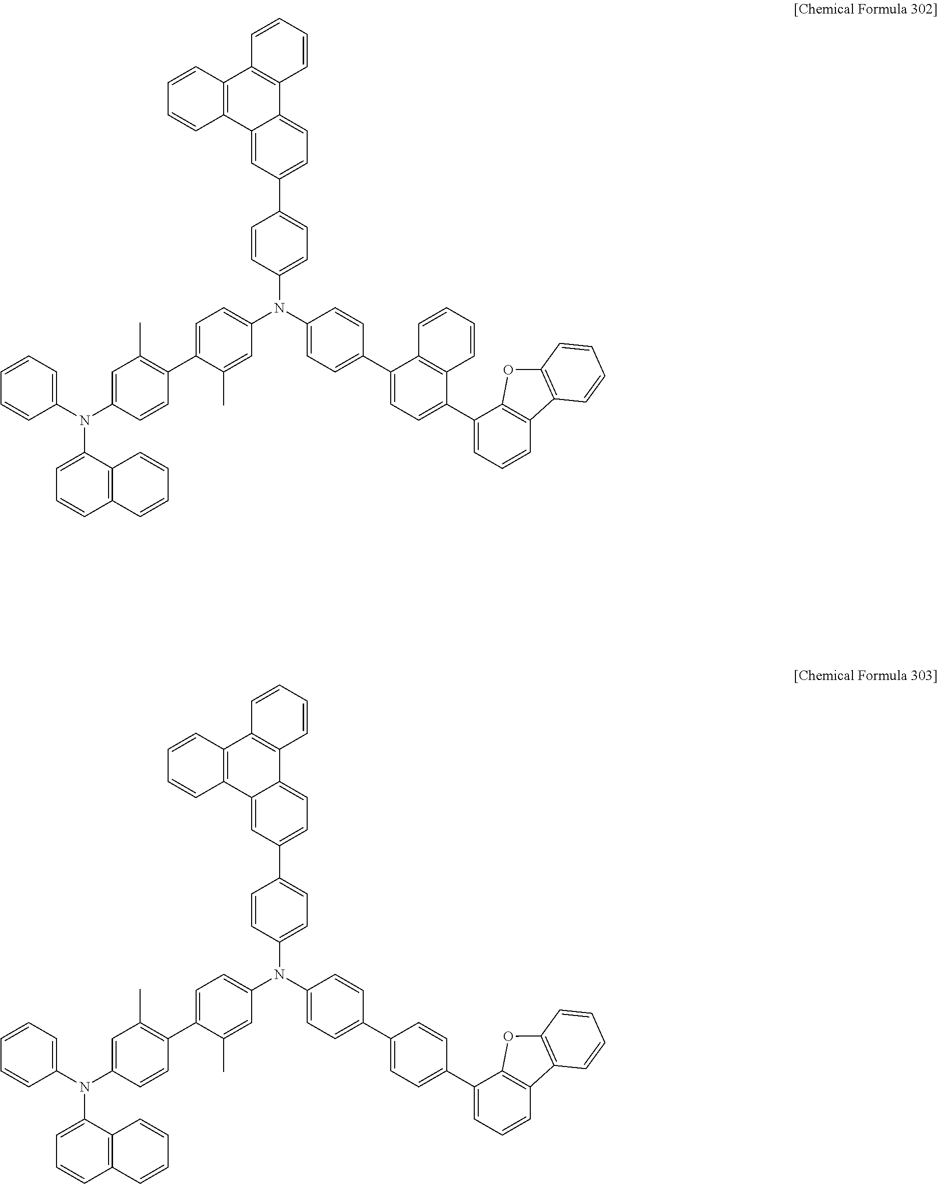 [SMILES: Cc1cc(N(c2ccc(-c3ccc4c5ccccc5c5ccccc5c4c3)cc2)c2ccc(-c3ccc(-c4cccc5c4oc4ccccc45)c4ccccc34)cc2)ccc1-c1ccc(N(c2ccccc2)c2cccc3ccccc23)cc1C ; Cc1cc(N(c2ccc(-c3ccc(-c4cccc5c4oc4ccccc45)cc3)cc2)c2ccc(-c3ccc4c5ccccc5c5ccccc5c4c3)cc2)ccc1-c1ccc(N(c2ccccc2)c2cccc3ccccc23)cc1C]

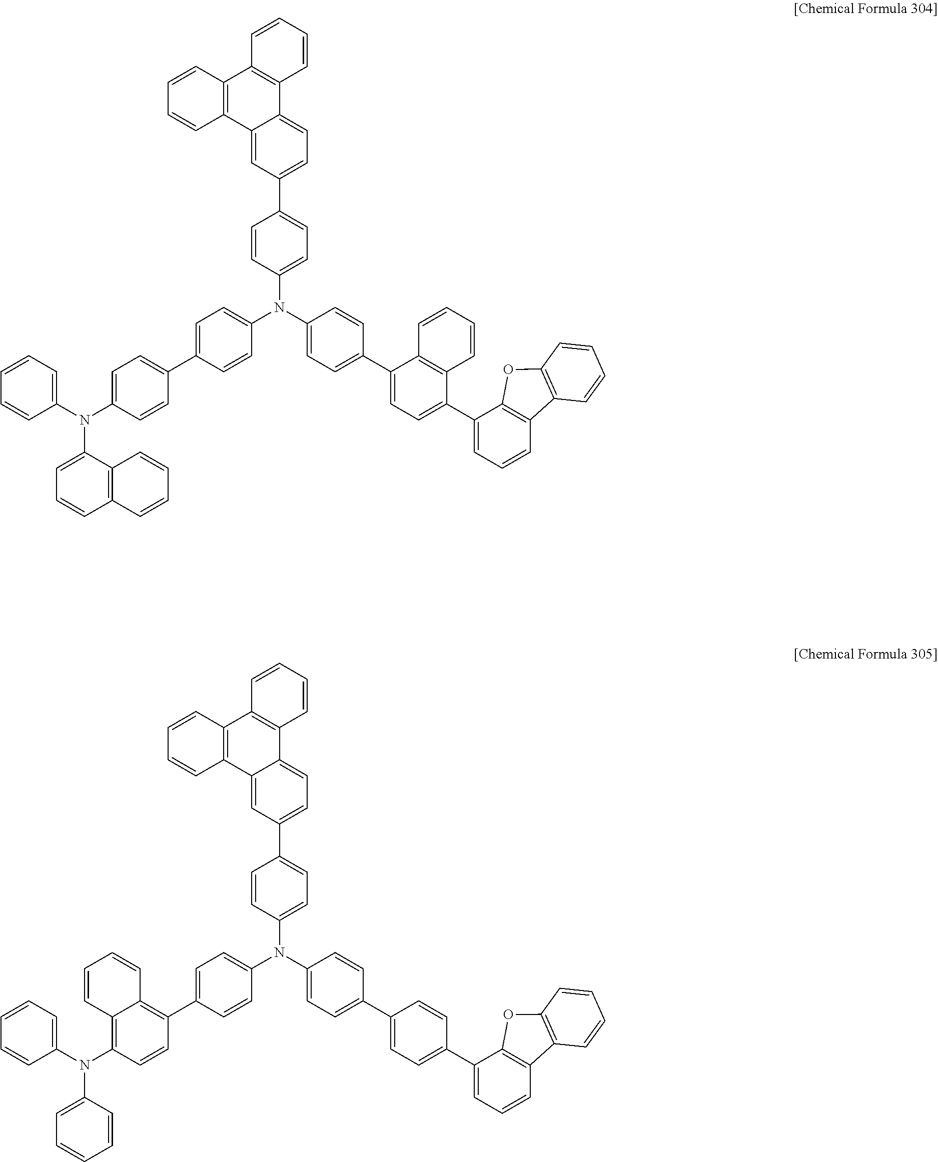 [SMILES: c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1 ; c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)c3ccccc23)cc1]

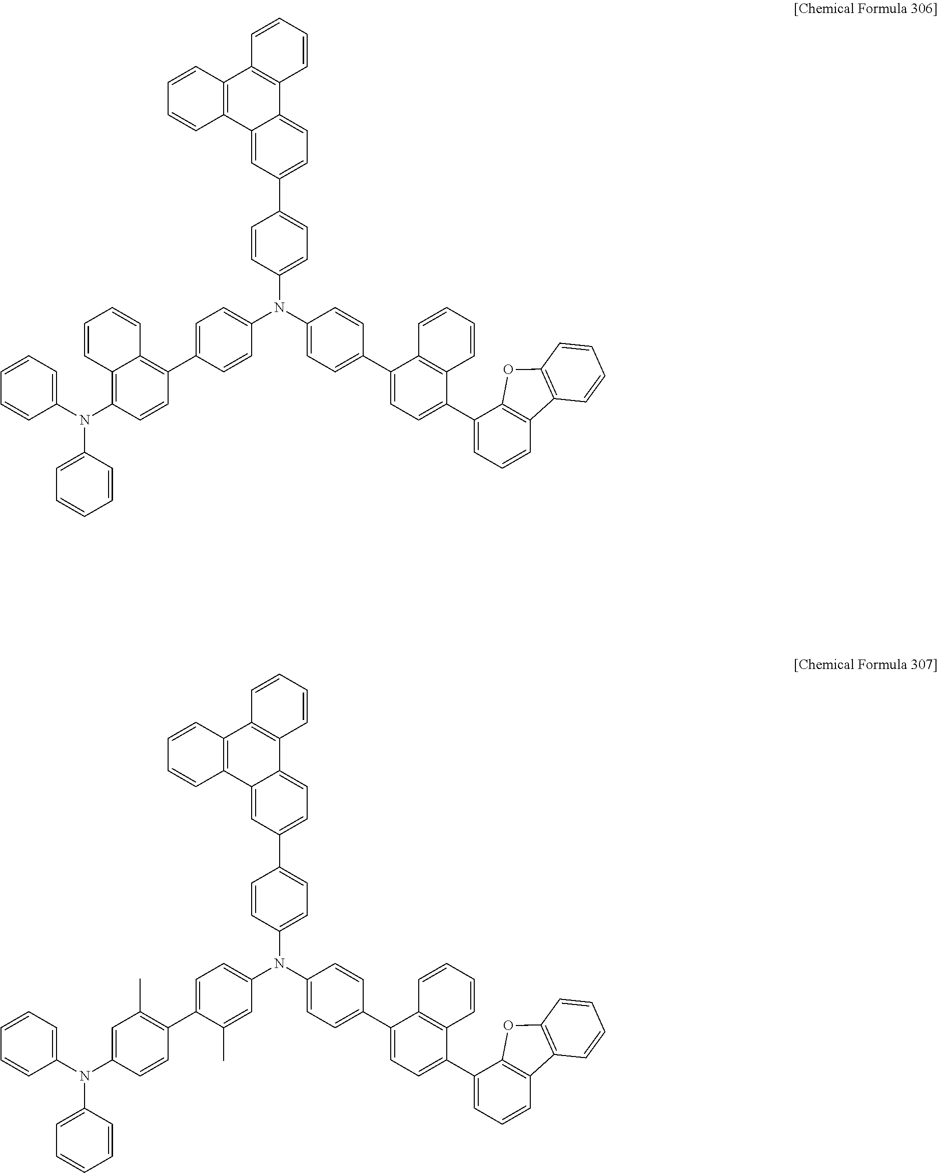 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4oc4ccccc45)c4ccccc34)cc2)cc1C]

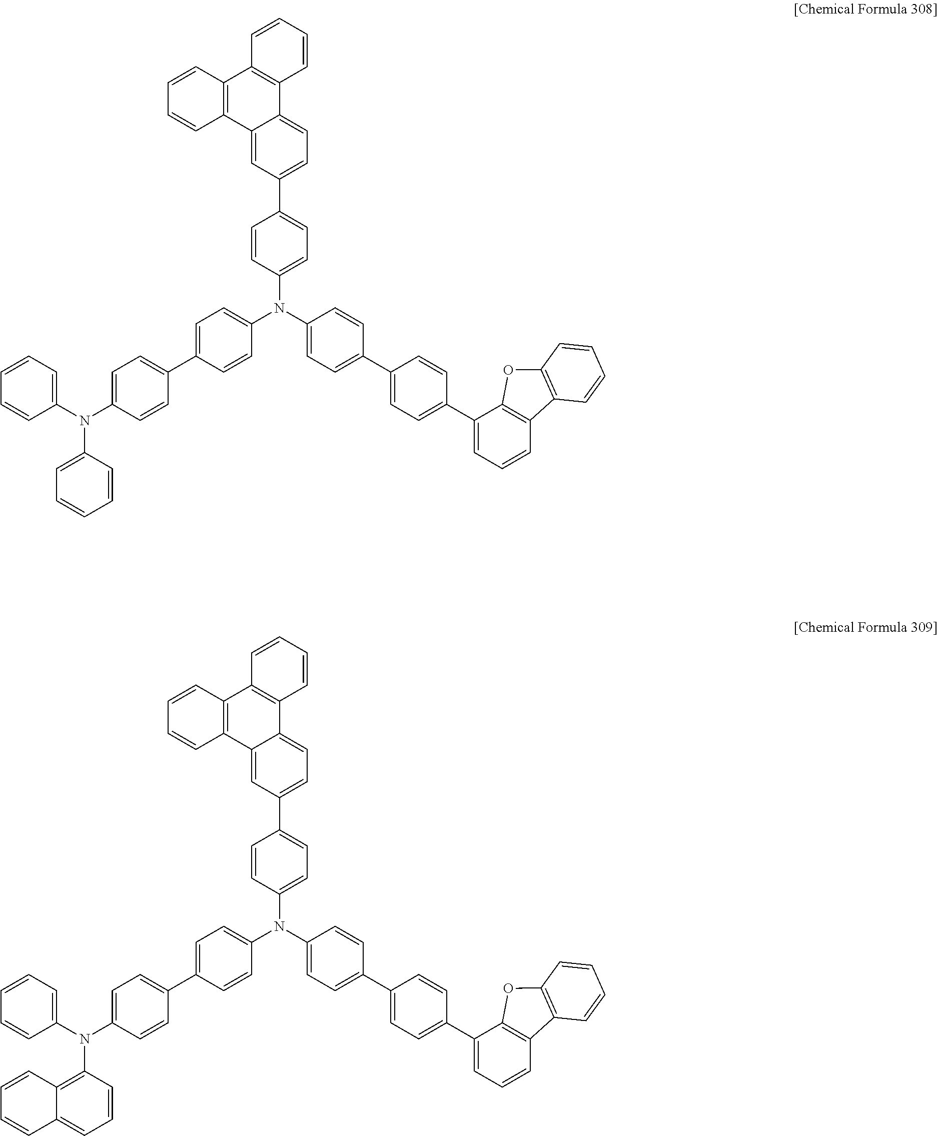 [SMILES: c1ccc(N(c2ccccc2)c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6cccc7c6oc6ccccc67)cc5)cc4)c4ccc(-c5ccc6c7ccccc7c7ccccc7c6c5)cc4)cc3)cc2)c2cccc3ccccc23)cc1]

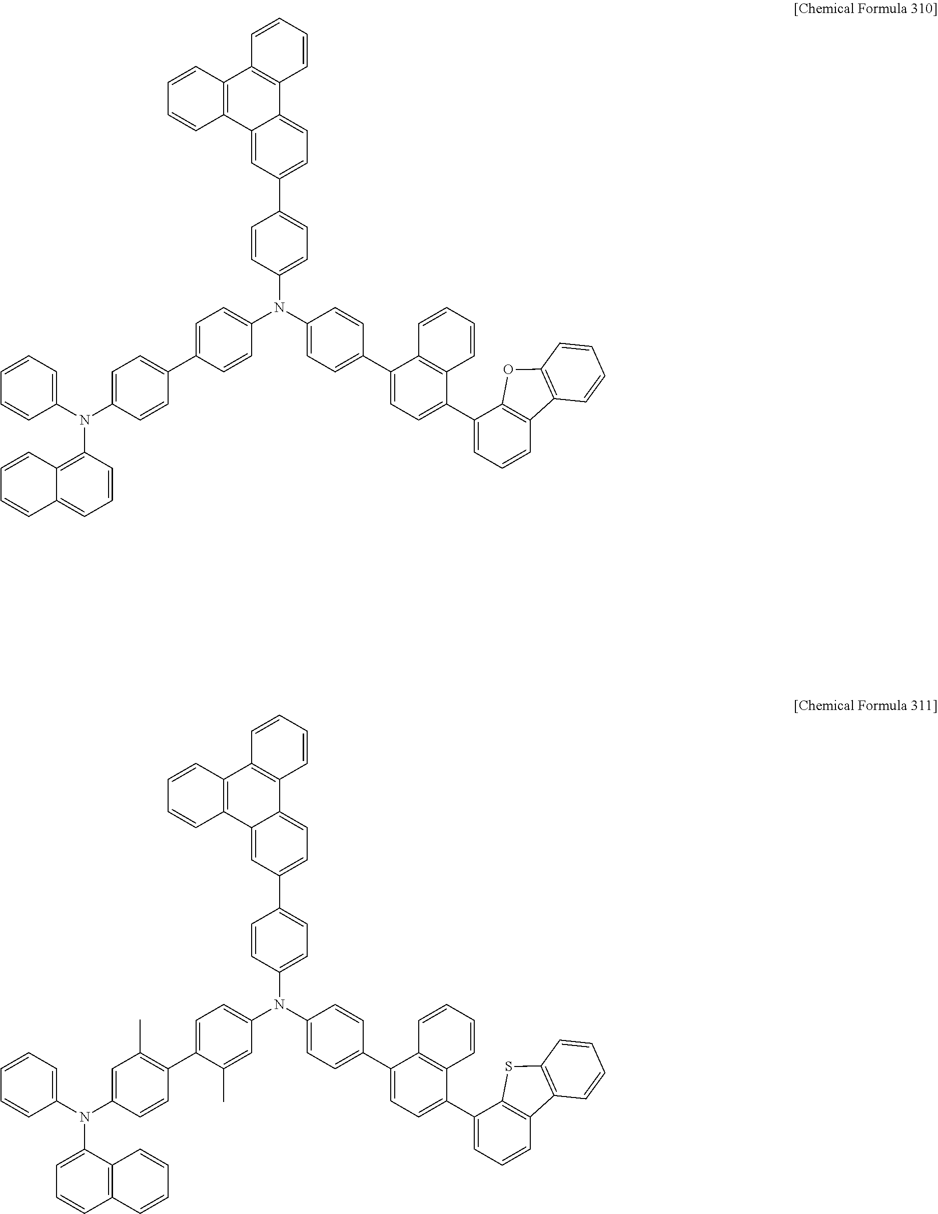 [SMILES: c1ccc(N(c2ccc(-c3ccc(N(c4ccc(-c5ccc6c7ccccc7c7ccccc7c6c5)cc4)c4ccc(-c5ccc(-c6cccc7c6oc6ccccc67)c6ccccc56)cc4)cc3)cc2)c2cccc3ccccc23)cc1 ; Cc1cc(N(c2ccc(-c3ccc4c5ccccc5c5ccccc5c4c3)cc2)c2ccc(-c3ccc(-c4cccc5c4sc4ccccc45)c4ccccc34)cc2)ccc1-c1ccc(N(c2ccccc2)c2cccc3ccccc23)cc1C]

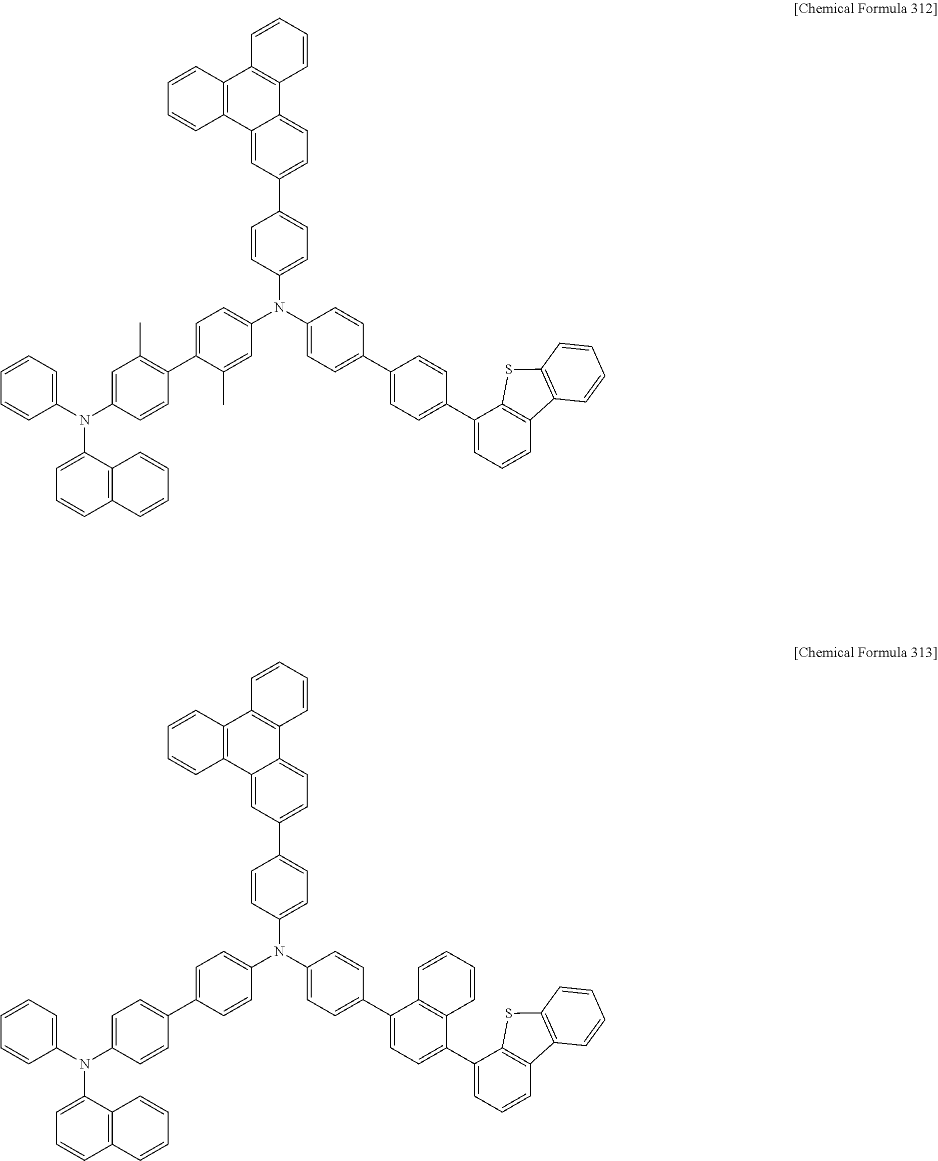 [SMILES: Cc1cc(N(c2ccc(-c3ccc(-c4cccc5c4sc4ccccc45)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1]

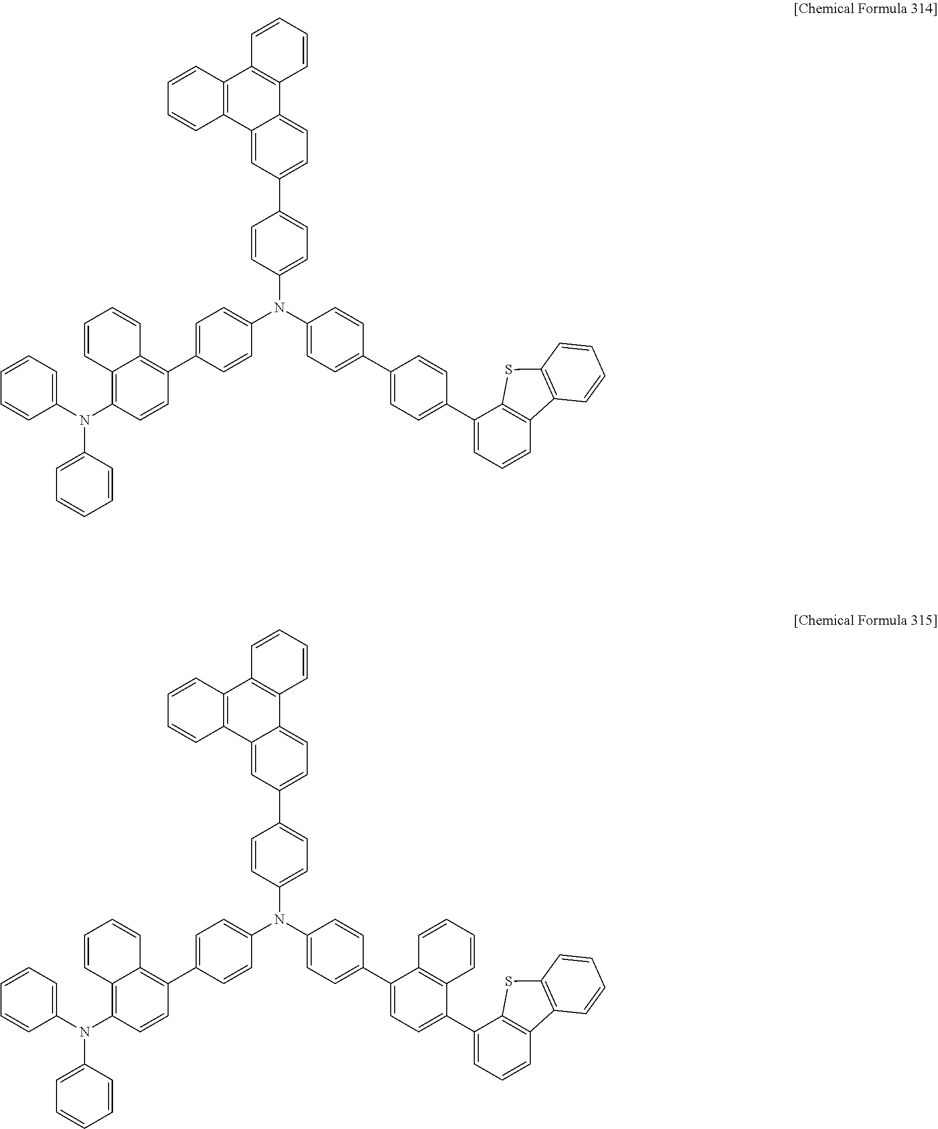 [SMILES: c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)c3ccccc23)cc1]

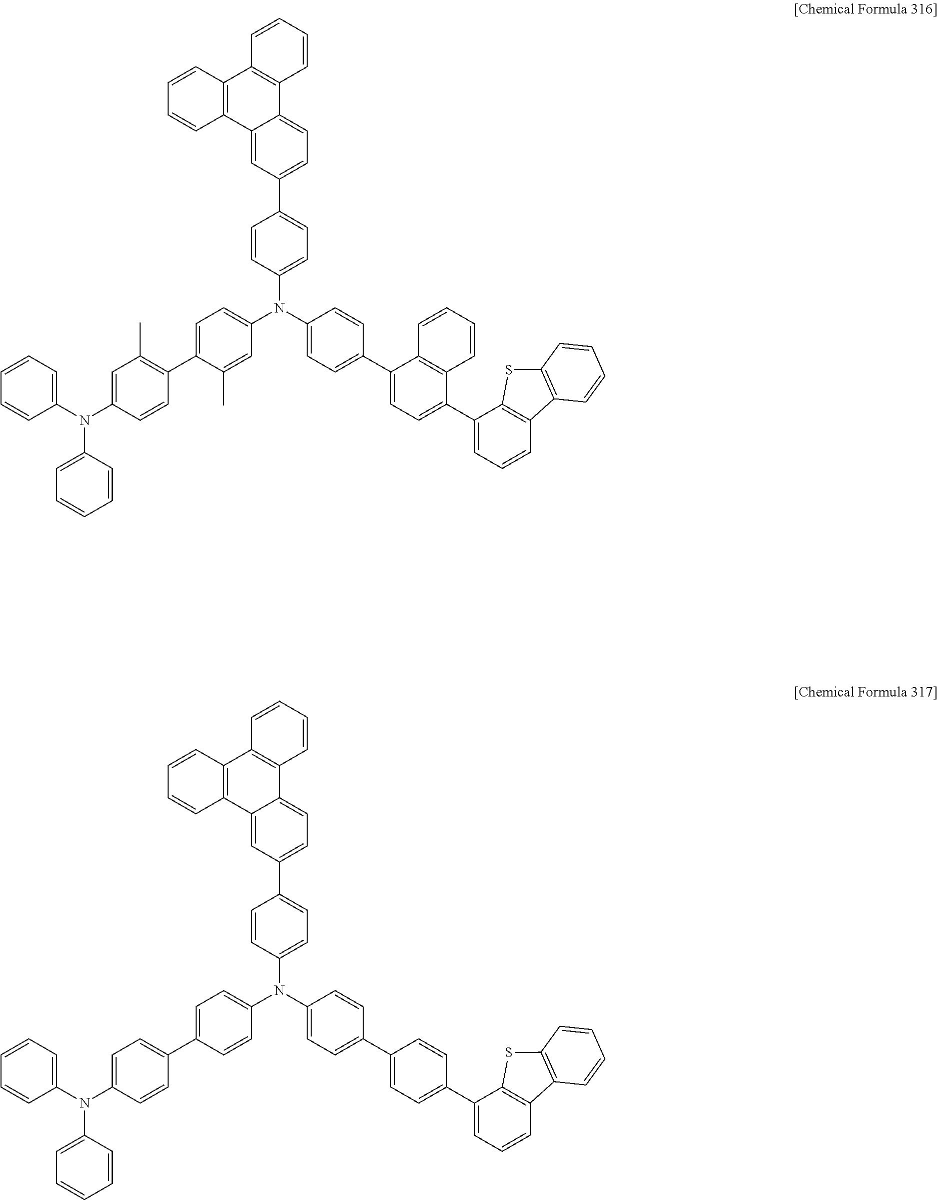 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4cccc5c4sc4ccccc45)c4ccccc34)cc2)cc1C ; c1ccc(N(c2ccccc2)c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)cc1]

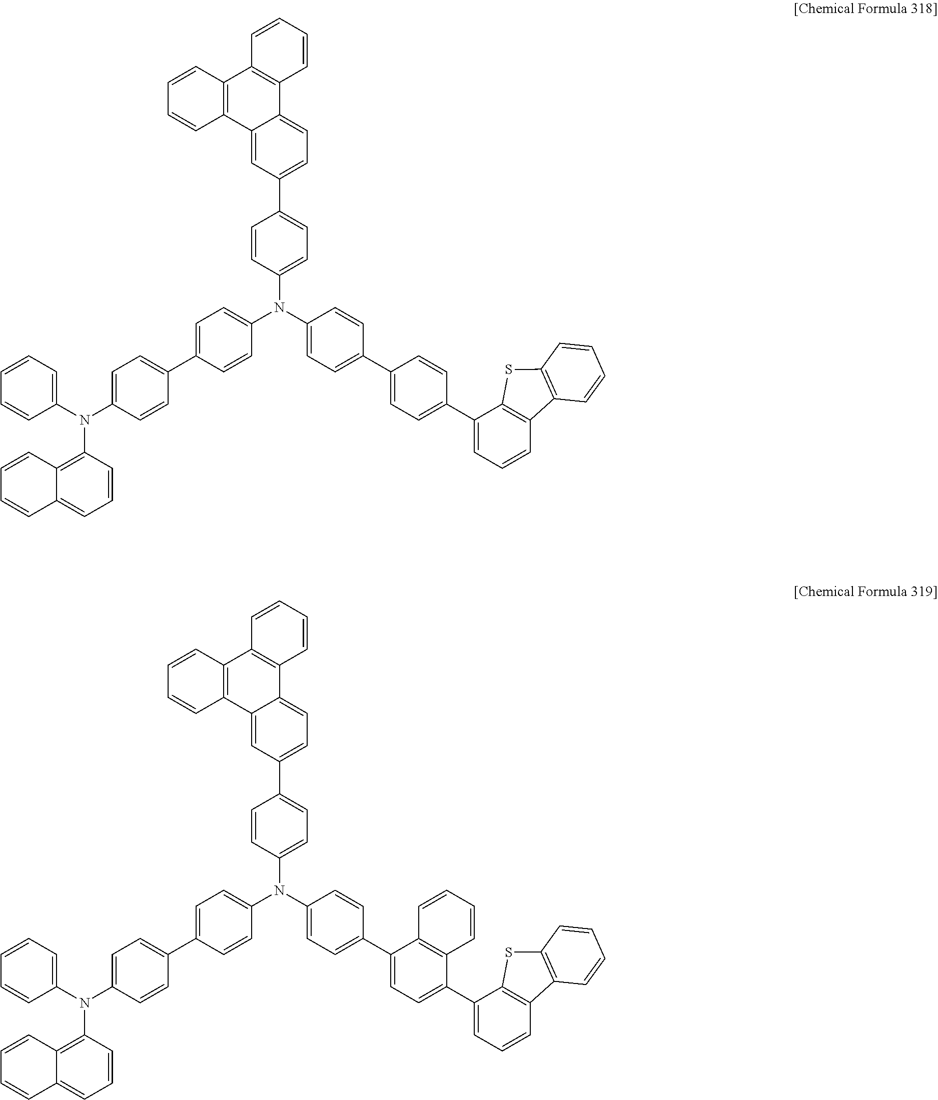 [SMILES: c1ccc(N(c2ccc(-c3ccc(N(c4ccc(-c5ccc(-c6cccc7c6sc6ccccc67)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6cccc7c6sc6ccccc67)c6ccccc56)cc4)cc3)cc2)c2cccc3ccccc23)cc1]

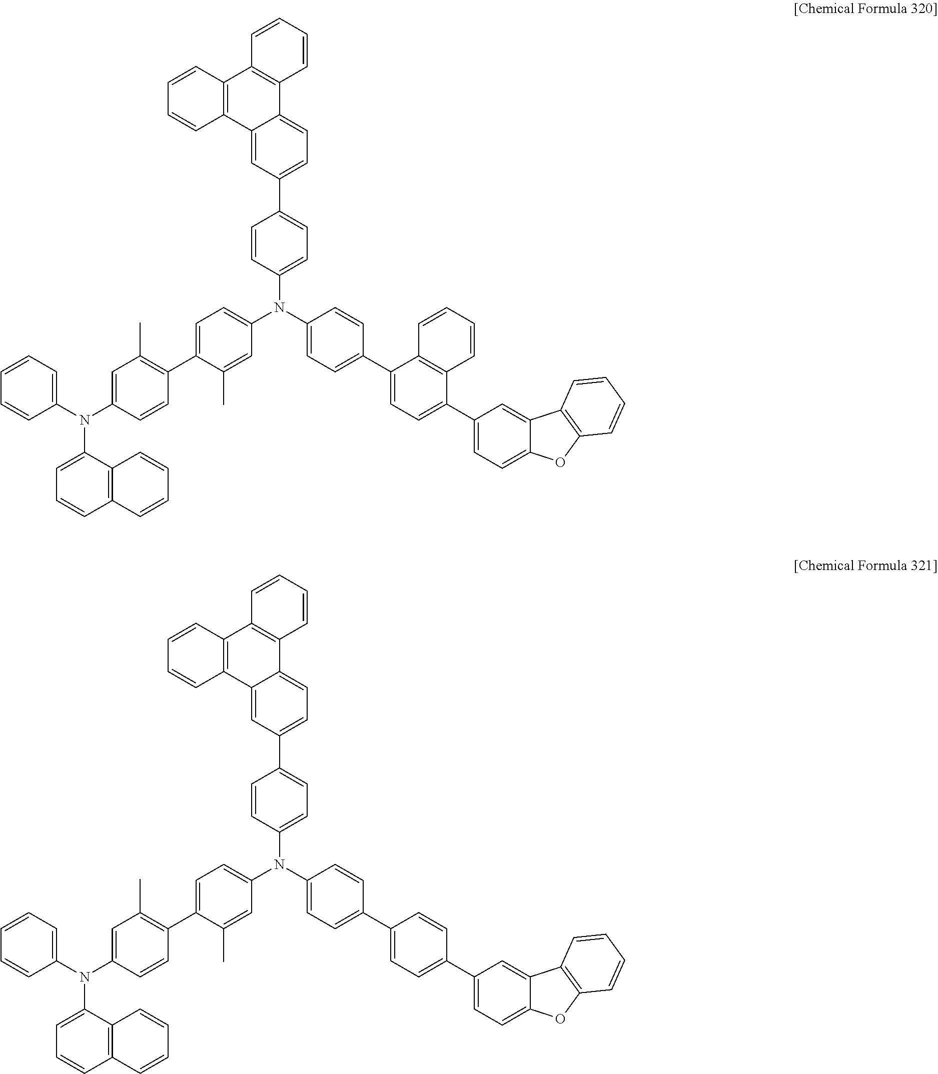 [SMILES: Cc1cc(N(c2ccc(-c3ccc4c5ccccc5c5ccccc5c4c3)cc2)c2ccc(-c3ccc(-c4ccc5oc6ccccc6c5c4)c4ccccc34)cc2)ccc1-c1ccc(N(c2ccccc2)c2cccc3ccccc23)cc1C ; Cc1cc(N(c2ccc(-c3ccc(-c4ccc5oc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C]

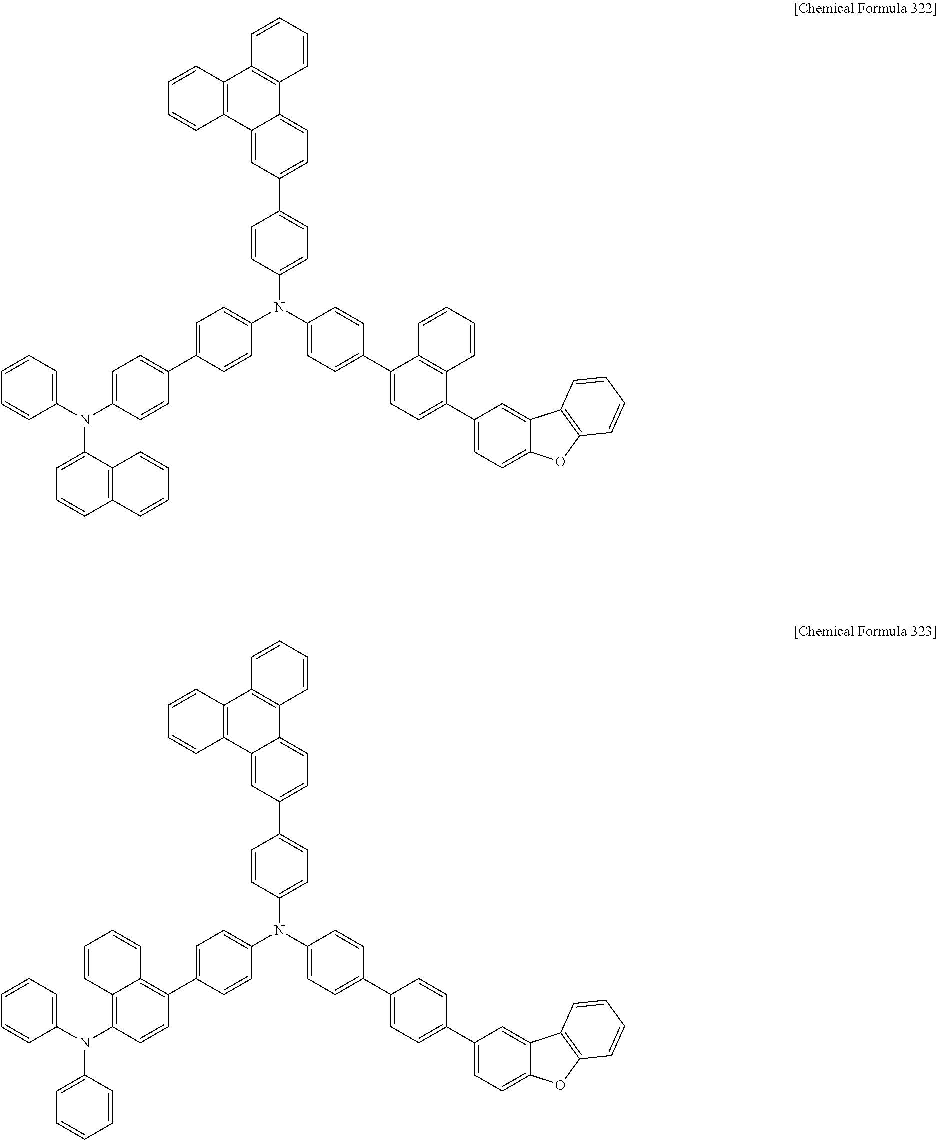 [SMILES: c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1 ; c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)c3ccccc23)cc1]

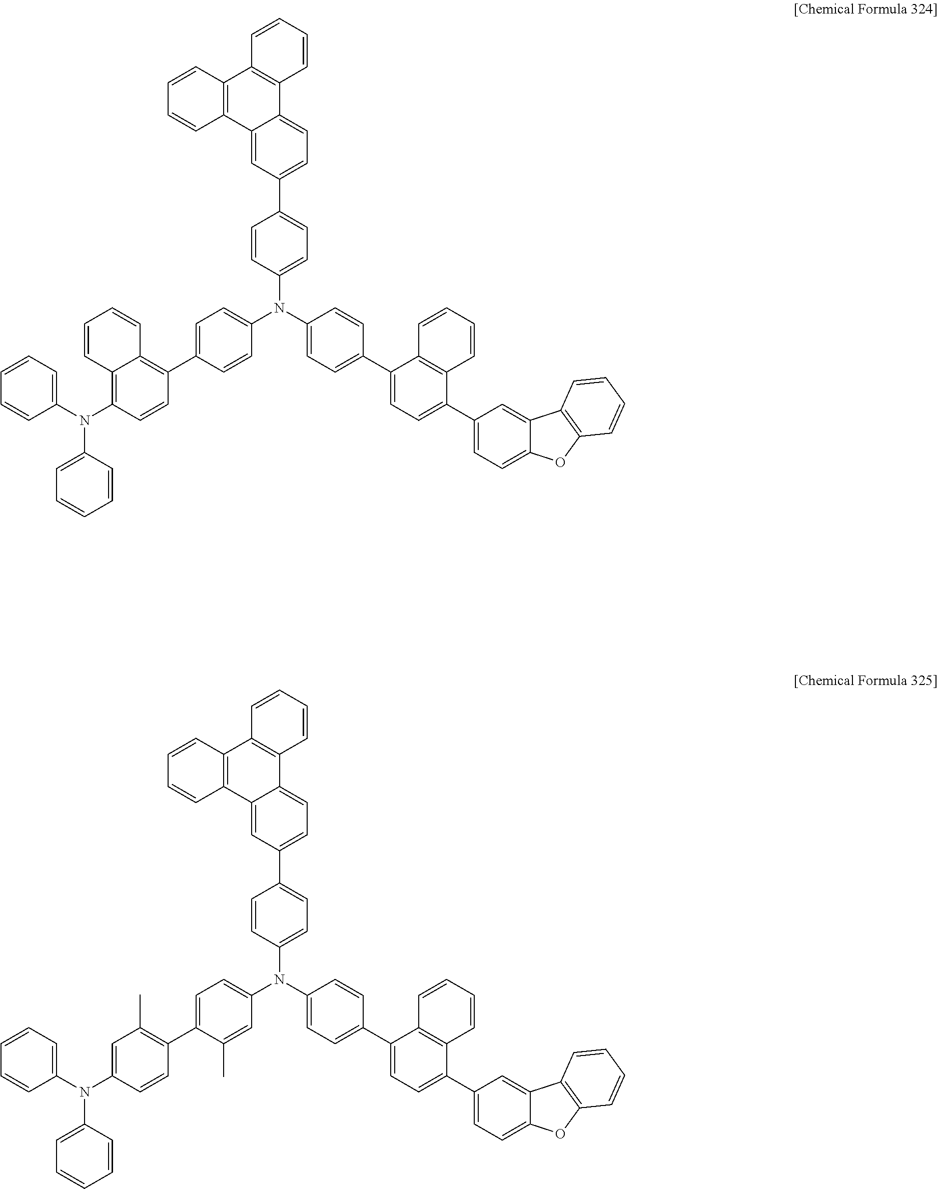 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)c3ccccc23)cc1 ; Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5oc6ccccc6c5c4)c4ccccc34)cc2)cc1C]

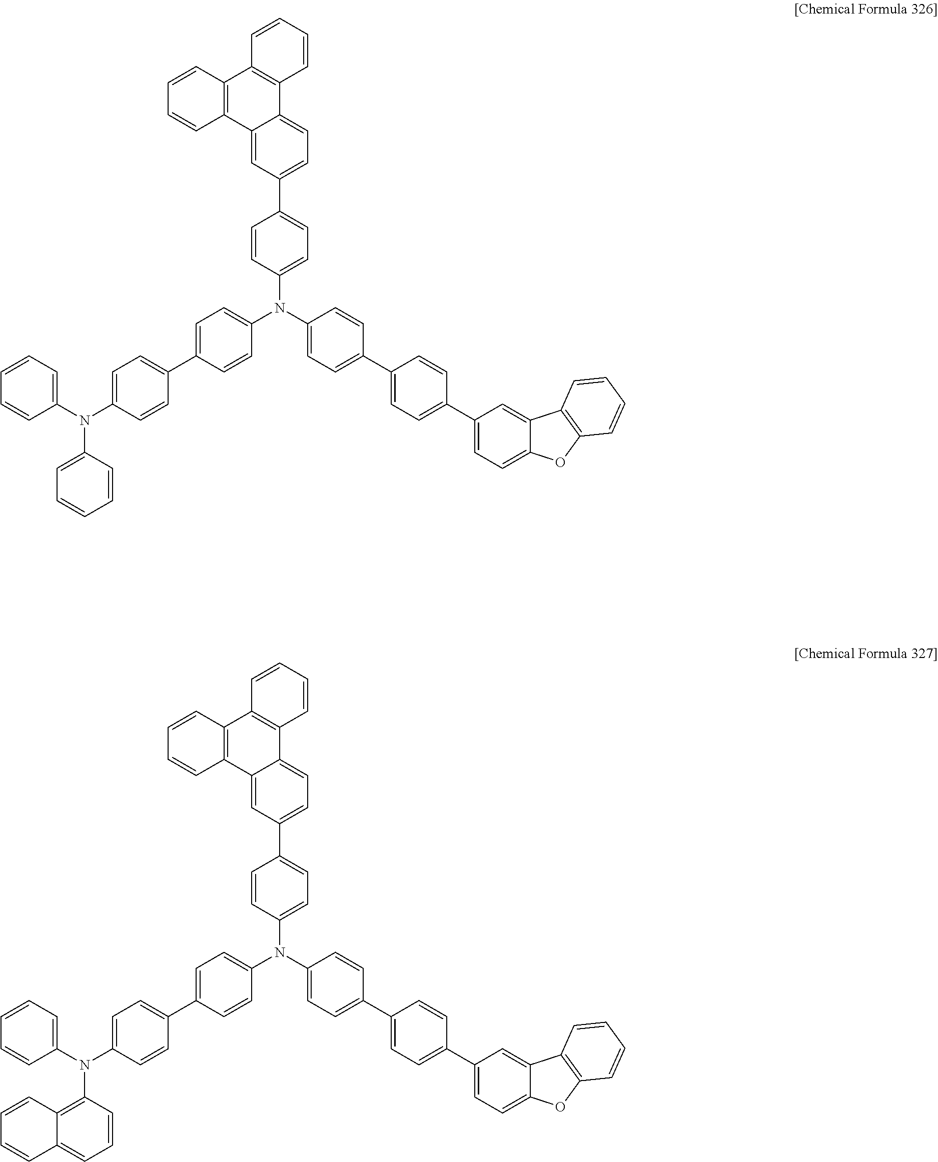 [SMILES: c1ccc(N(c2ccccc2)c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)cc1 ; c1ccc(N(c2ccc(-c3ccc(N(c4ccc(-c5ccc(-c6ccc7oc8ccccc8c7c6)cc5)cc4)c4ccc(-c5ccc6c7ccccc7c7ccccc7c6c5)cc4)cc3)cc2)c2cccc3ccccc23)cc1]

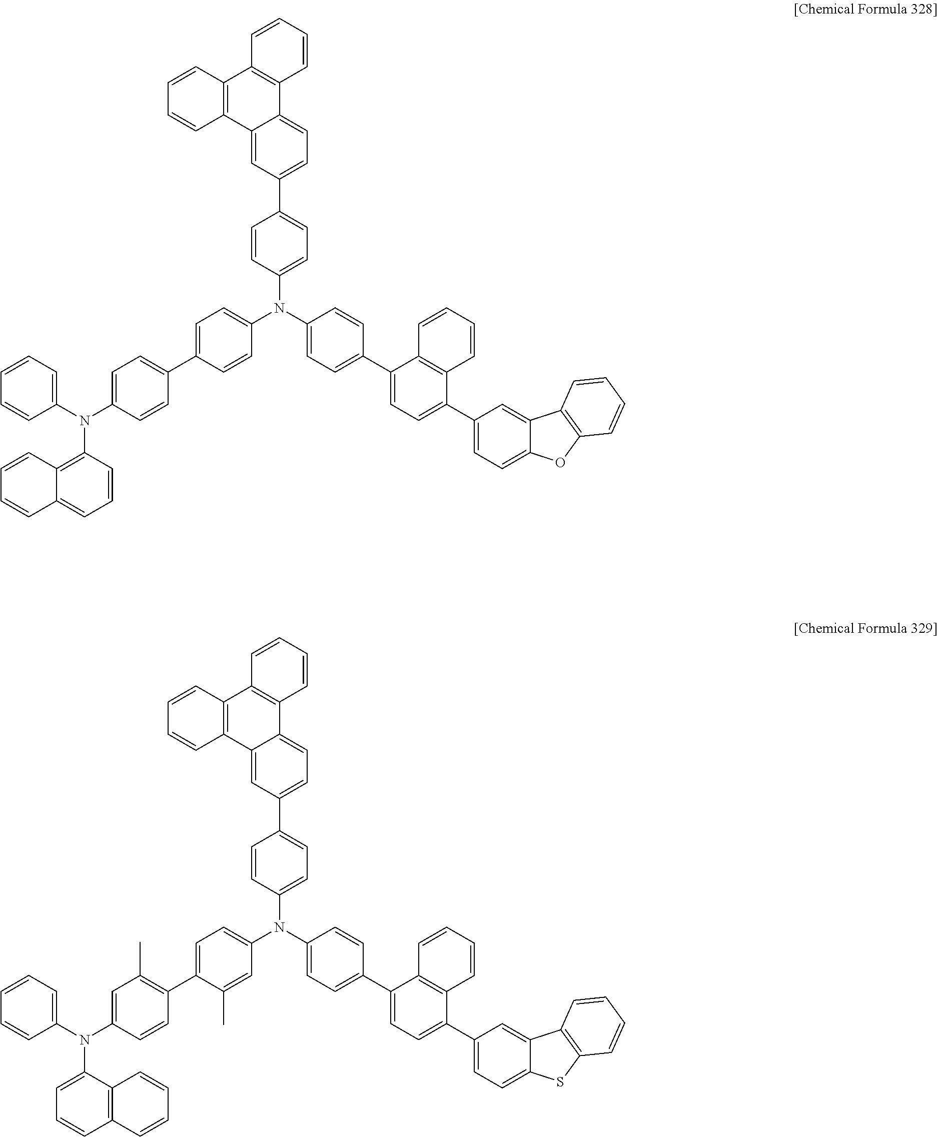 [SMILES: c1ccc(N(c2ccc(-c3ccc(N(c4ccc(-c5ccc6c7ccccc7c7ccccc7c6c5)cc4)c4ccc(-c5ccc(-c6ccc7oc8ccccc8c7c6)c6ccccc56)cc4)cc3)cc2)c2cccc3ccccc23)cc1 ; Cc1cc(N(c2ccc(-c3ccc4c5ccccc5c5ccccc5c4c3)cc2)c2ccc(-c3ccc(-c4ccc5sc6ccccc6c5c4)c4ccccc34)cc2)ccc1-c1ccc(N(c2ccccc2)c2cccc3ccccc23)cc1C]

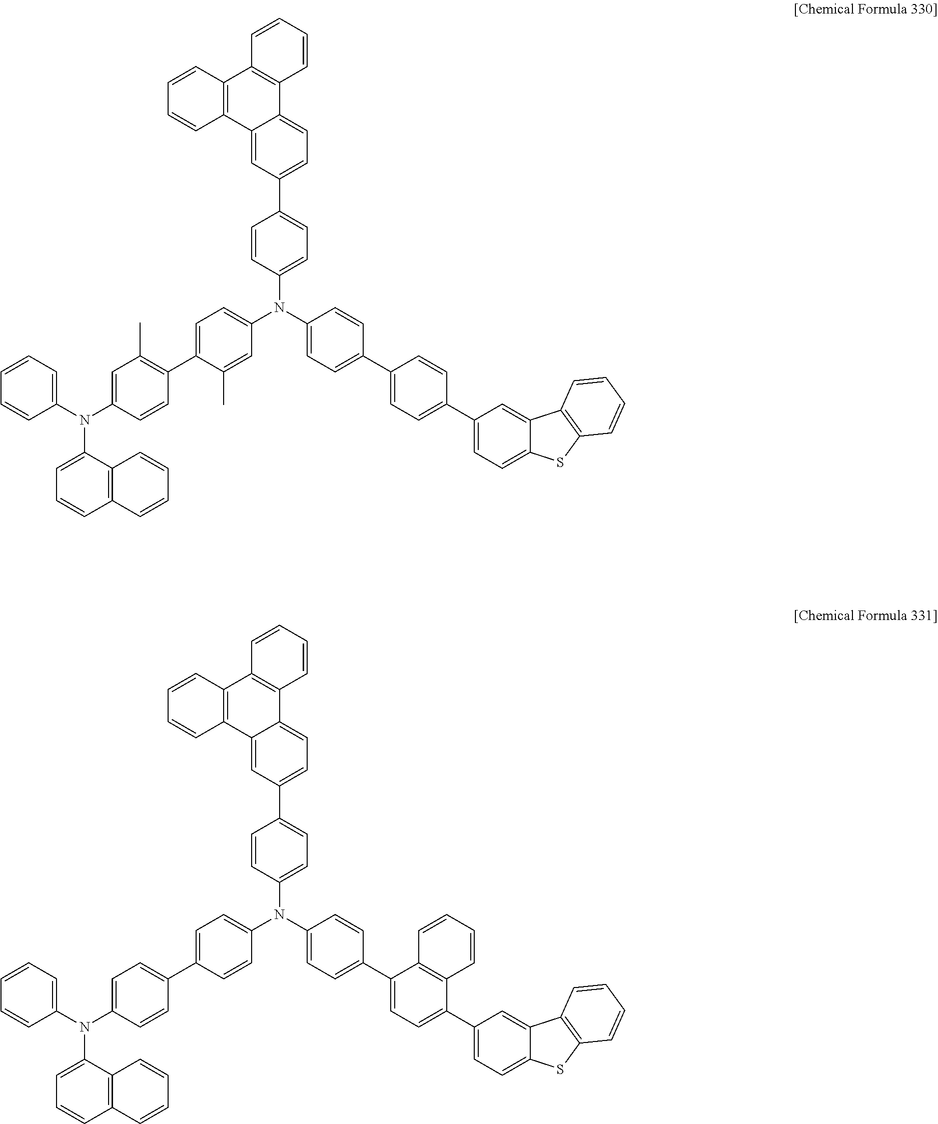 [SMILES: Cc1cc(N(c2ccc(-c3ccc(-c4ccc5sc6ccccc6c5c4)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1]

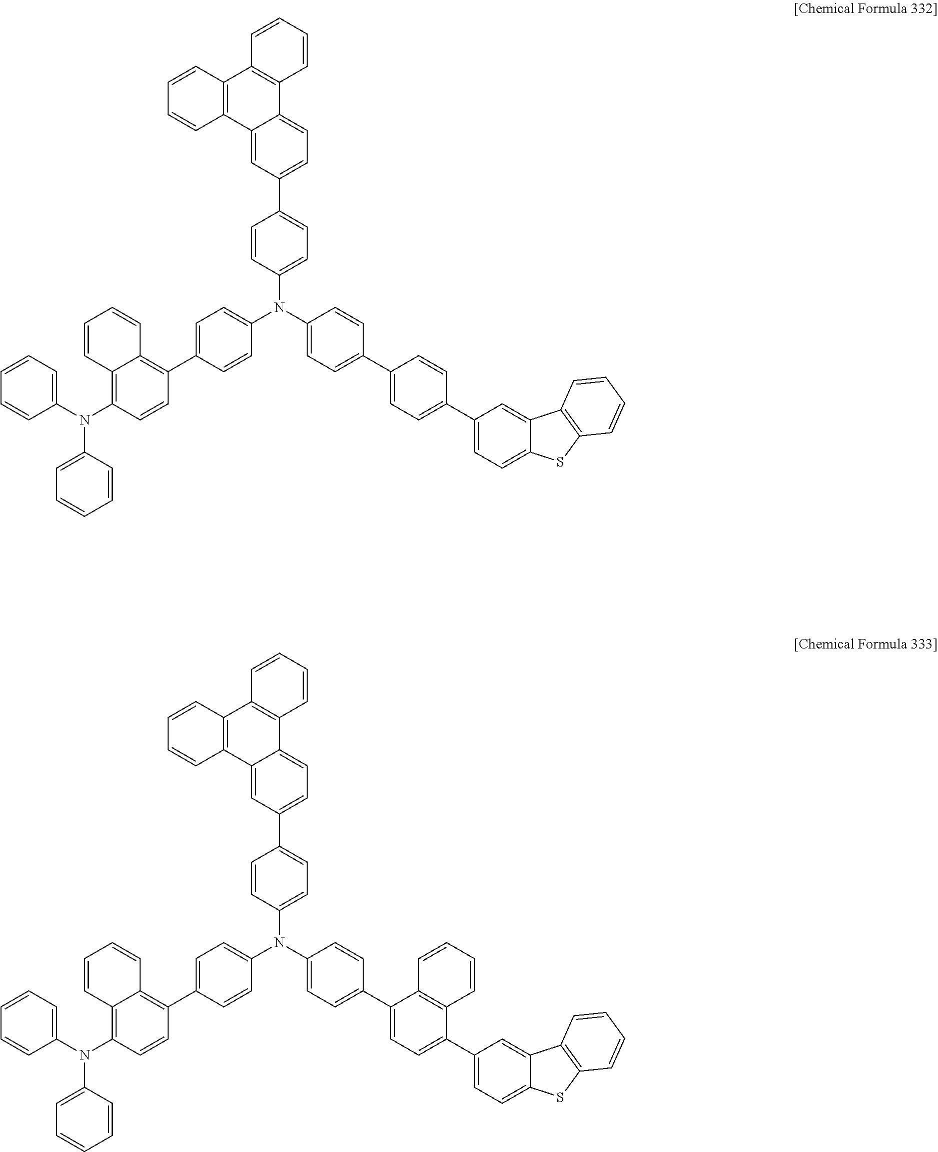 [SMILES: c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)c3ccccc23)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)c3ccccc23)cc1]

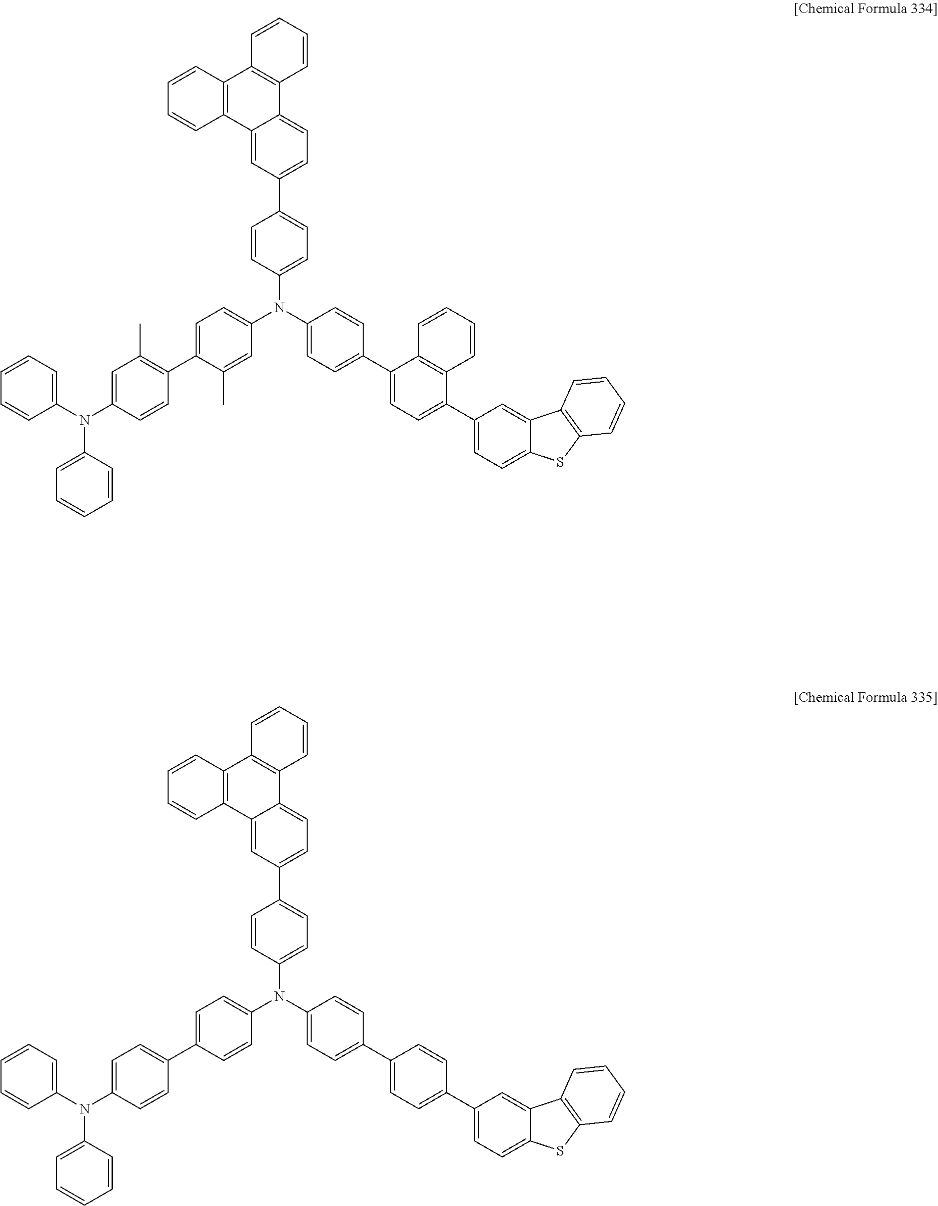 [SMILES: Cc1cc(N(c2ccccc2)c2ccccc2)ccc1-c1ccc(N(c2ccc(-c3ccc4c5ccccc5c5ccccc5c4c3)cc2)c2ccc(-c3ccc(-c4ccc5sc6ccccc6c5c4)c4ccccc34)cc2)cc1C ; c1ccc(N(c2ccccc2)c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)cc1]

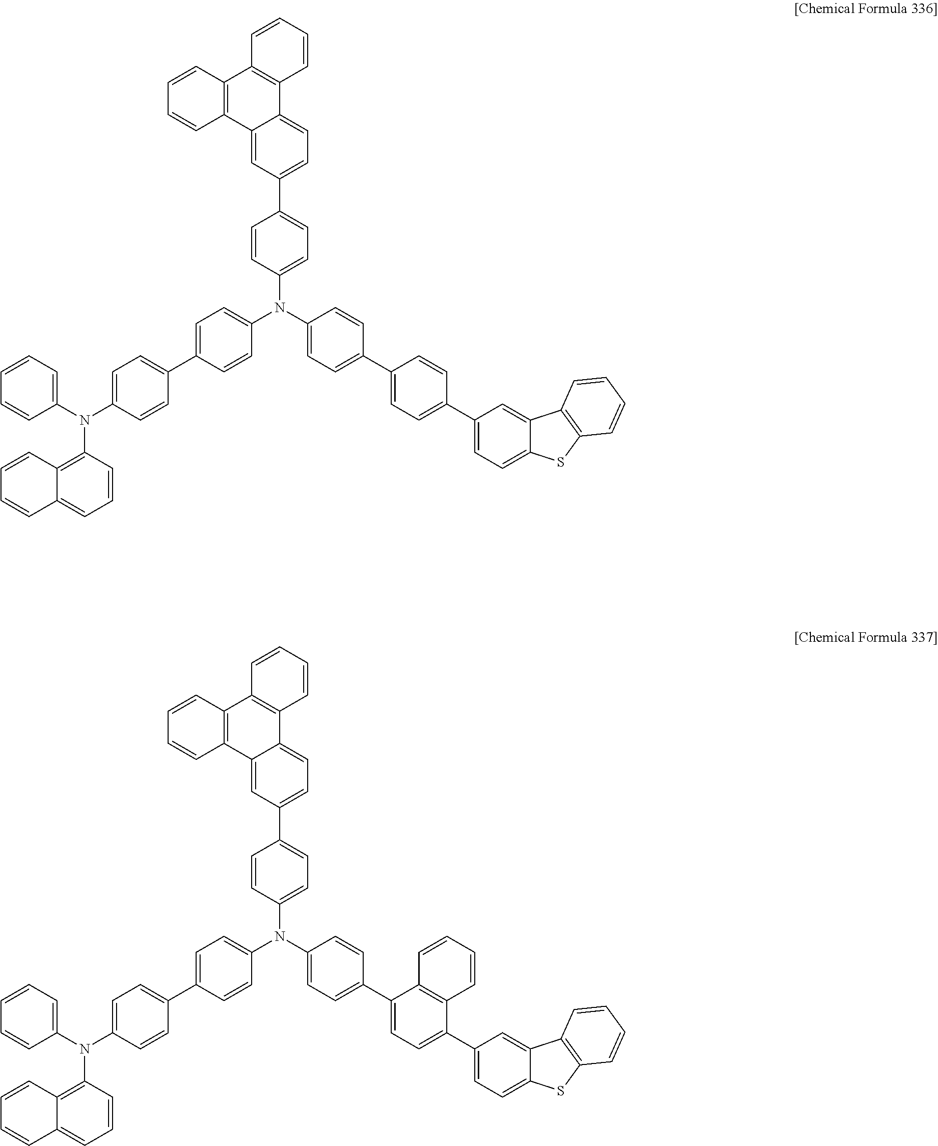 [SMILES: c1ccc(N(c2ccc(-c3ccc(N(c4ccc(-c5ccc(-c6ccc7sc8ccccc8c7c6)cc5)cc4)c4ccc(-c5ccc6c7ccccc7c7ccccc7c6c5)cc4)cc3)cc2)c2cccc3ccccc23)cc1 ; c1ccc(N(c2ccc(-c3ccc(N(c4ccc(-c5ccc6c7ccccc7c7ccccc7c6c5)cc4)c4ccc(-c5ccc(-c6ccc7sc8ccccc8c7c6)c6ccccc56)cc4)cc3)cc2)c2cccc3ccccc23)cc1]

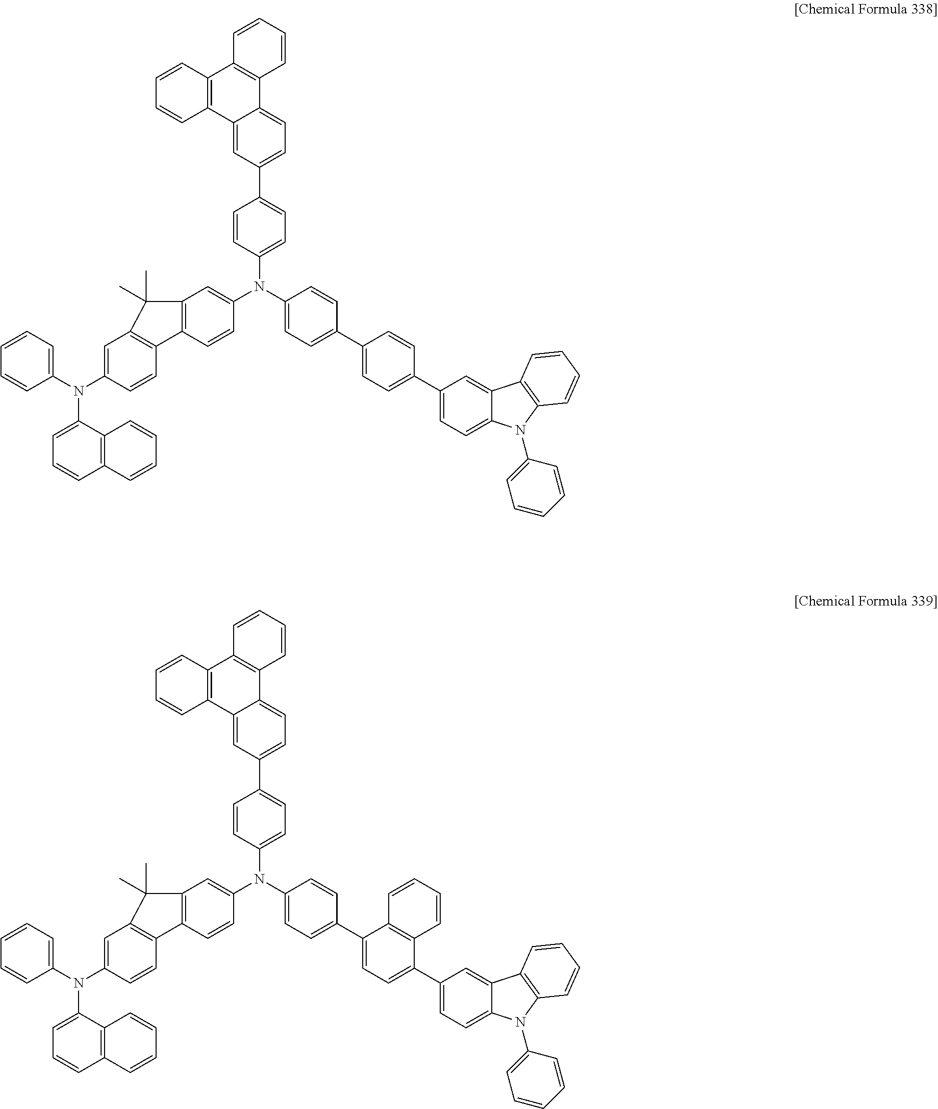 [SMILES: CC1(C)c2cc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)ccc2-c2ccc(N(c3ccccc3)c3cccc4ccccc34)cc21 ; CC1(C)c2cc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)ccc2-c2ccc(N(c3ccccc3)c3cccc4ccccc34)cc21]

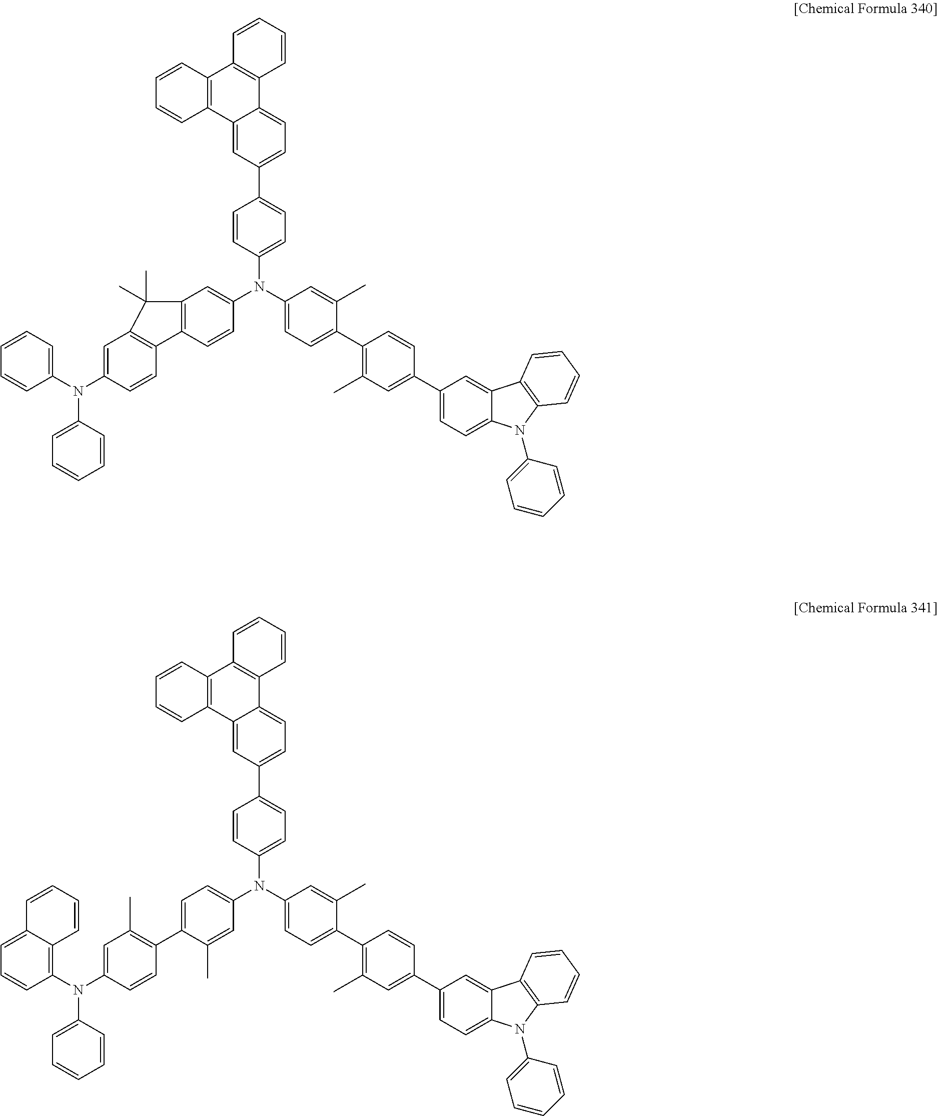 [SMILES: Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc3c(c2)C(C)(C)c2cc(N(c4ccccc4)c4ccccc4)ccc2-3)cc1C ; Cc1cc(-c2ccc3c(c2)c2ccccc2n3-c2ccccc2)ccc1-c1ccc(N(c2ccc(-c3ccc4c5ccccc5c5ccccc5c4c3)cc2)c2ccc(-c3ccc(N(c4ccccc4)c4cccc5ccccc45)cc3C)c(C)c2)cc1C]

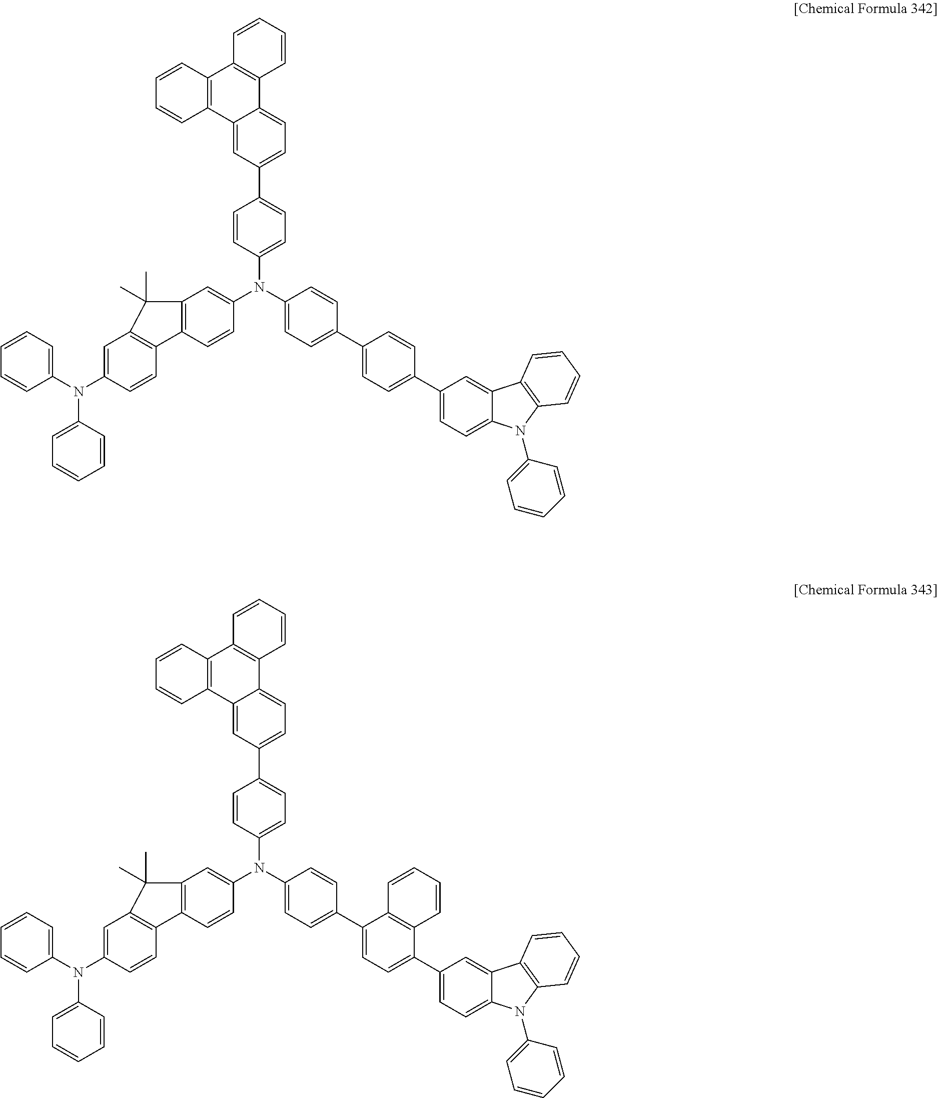 [SMILES: CC1(C)c2cc(N(c3ccccc3)c3ccccc3)ccc2-c2ccc(N(c3ccc(-c4ccc(-c5ccc6c(c5)c5ccccc5n6-c5ccccc5)cc4)cc3)c3ccc(-c4ccc5c6ccccc6c6ccccc6c5c4)cc3)cc21 ; CC1(C)c2cc(N(c3ccccc3)c3ccccc3)ccc2-c2ccc(N(c3ccc(-c4ccc5c6ccccc6c6ccccc6c5c4)cc3)c3ccc(-c4ccc(-c5ccc6c(c5)c5ccccc5n6-c5ccccc5)c5ccccc45)cc3)cc21]

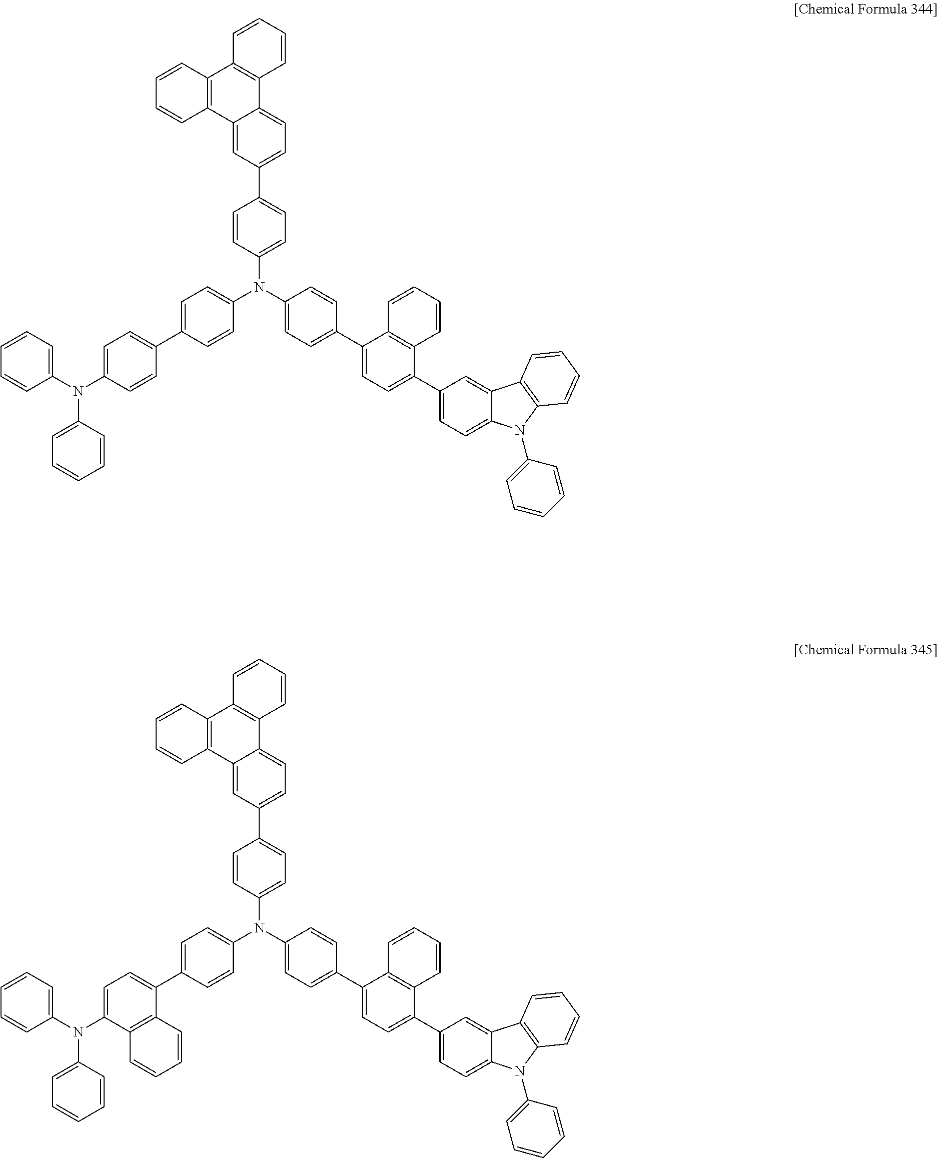 [SMILES: c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)cc2)cc1 ; c1ccc(N(c2ccccc2)c2ccc(-c3ccc(N(c4ccc(-c5ccc6c7ccccc7c7ccccc7c6c5)cc4)c4ccc(-c5ccc(-c6ccc7c(c6)c6ccccc6n7-c6ccccc6)c6ccccc56)cc4)cc3)c3ccccc23)cc1]

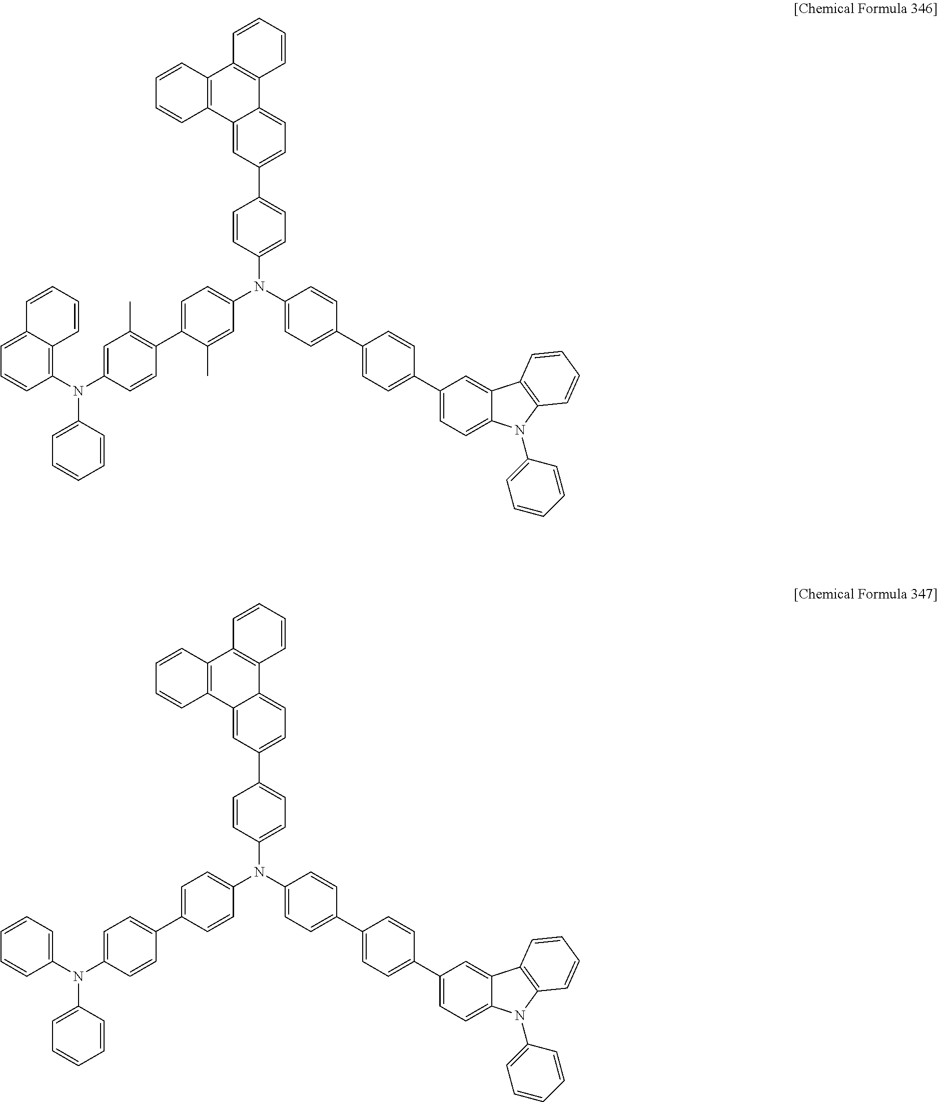 [SMILES: Cc1cc(N(c2ccc(-c3ccc(-c4ccc5c(c4)c4ccccc4n5-c4ccccc4)cc3)cc2)c2ccc(-c3ccc4c5ccccc5c5ccccc5c4c3)cc2)ccc1-c1ccc(N(c2ccccc2)c2cccc3ccccc23)cc1C ; c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)cc1]

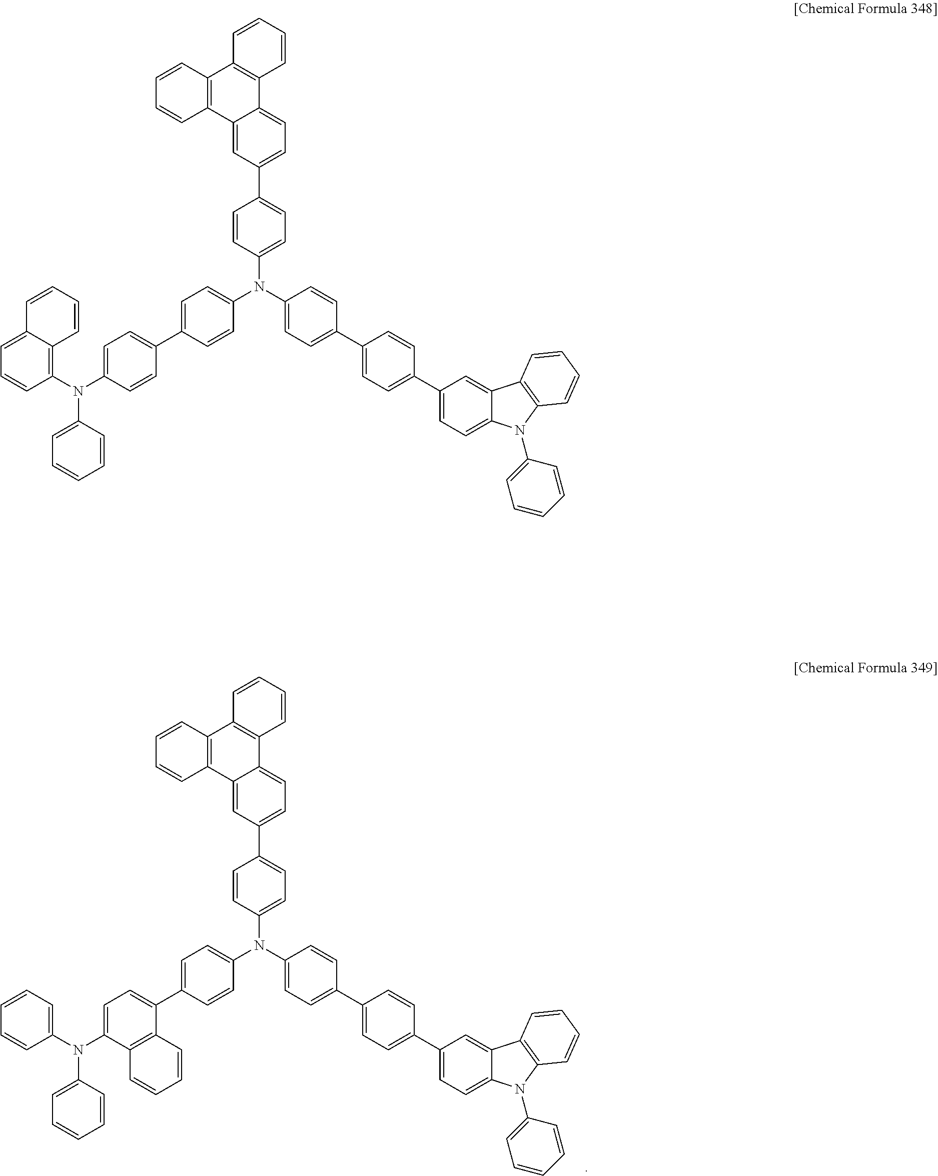 [SMILES: c1ccc(N(c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)cc2)c2cccc3ccccc23)cc1 ; c1ccc(N(c2ccccc2)c2ccc(-c3ccc(N(c4ccc(-c5ccc(-c6ccc7c(c6)c6ccccc6n7-c6ccccc6)cc5)cc4)c4ccc(-c5ccc6c7ccccc7c7ccccc7c6c5)cc4)cc3)c3ccccc23)cc1]

12. The compound as claimed in claim 1, wherein the organic photoelectric device is selected from the group of an organic light emitting diode, an organic solar cell, an organic transistor, an organic photo conductor drum, and an organic memory device.

13. An organic light emitting diode (OLED), comprising an anode, a cathode, and an organic thin layer between the anode and the cathode, the organic thin layer including the compound for an organic photoelectric device as claimed in claim 1.

14. The OLED as claimed in claim 13, wherein the organic thin layer includes one or more of an emission layer, a hole transport layer (HTL), a hole injection layer (HIL), an electron transport layer (ETL), an electron injection layer (EIL), or a hole blocking layer.

15. The OLED as claimed in claim 13, wherein the compound for an organic photoelectric device is included in a hole transport layer (HTL), or a hole injection layer (HIL).

16. The OLED as claimed in claim 13, wherein the compound for an organic photoelectric device is included in an emission layer.

17. The OLED as claimed in claim 13, wherein the compound for an organic photoelectric device is a phosphorescent or fluorescent host material in an emission layer.

18. The OLED as claimed in claim 13, wherein the compound for an organic photoelectric device is a fluorescent blue dopant material in an emission layer.

19. A display device comprising the OLED as claimed in claim 13.
